SEQUENCE LISTING (223 sequences)

SEQ ID NO: 1 - skipped

SEQ ID NO: 2 - DNA, 23 bp, synthetic construct
  misc_feature 1..23: note = Synthetic: miR122 binding site
  source 1..23: mol_type = other DNA
acaaacaccattgtcacactcca

SEQ ID NO: 3 - DNA, 71 bp, synthetic construct
  misc_feature 1..71: note = Synthetic: miR122 binding site
  source 1..71: mol_type = other DNA
acaaacaccattgtcacactccacacaaacaccattgtcacactccacacaaacaccattgtcacactcca

SEQ ID NO: 4 - DNA, 22 bp, synthetic construct
  misc_feature 1..22: note = Synthetic: miR-1 binding site
  source 1..22: mol_type = other DNA
atacatacttctttacattcca

SEQ ID NO: 5 - DNA, 23 bp, synthetic construct
  misc_feature 1..23: note = Synthetic: miR-142-3p binding site
  source 1..23: mol_type = other DNA
tccataaagtaggaaacactaca

SEQ ID NO: 6 - DNA, 22 bp, synthetic construct
  misc_feature 1..22: note = Synthetic: miR-183 binding site
  source 1..22: mol_type = other DNA
agtgaattctaccagtgccata

SEQ ID NO: 7 - DNA, 24 bp, synthetic construct
  misc_feature 1..24: note = Synthetic: miR-182 binding site
  source 1..24: mol_type = other DNA
agtgtgagttctaccattgccaaa

SEQ ID NO: 8 - DNA, 23 bp, synthetic construct
  misc_feature 1..23: note = Synthetic: miR-96 binding site
  source 1..23: mol_type = other DNA
agcaaaaatgtgctagtgccaaa

SEQ ID NO: 9 - DNA, 15 bp, synthetic construct
  misc_feature 1..15: note = Synthetic: minicistron
  source 1..15: mol_type = other DNA
atgaacaaagattaa

SEQ ID NO: 10 - DNA, 18 bp, synthetic construct
  misc_feature 1..18: note = Synthetic: minicistron
  source 1..18: mol_type = other DNA
atgcctcaatgctactag

SEQ ID NO: 11 - DNA, 20 bp, synthetic construct
  misc_feature 1..20: note = Synthetic: forward primer
  source 1..20: mol_type = other DNA
tacggtaaactgcccacttg

SEQ ID NO: 12 - DNA, 22 bp, synthetic construct
  misc_feature 1..22: note = Synthetic: reverse primer
  source 1..22: mol_type = other DNA
gtcccataaggtcatgtactgg

SEQ ID NO: 13 - DNA, 22 bp, synthetic construct
  misc_feature 1..22: note = Synthetic: probe primer
  source 1..22: mol_type = other DNA
gtcccataaggtcatgtactgg

SEQ ID NO: 14-20 - skipped (7 entries)

SEQ ID NO: 21 - RNA, 11 nt, synthetic construct
  misc_feature 1..11: note = Synthetic: modified Kozak
  source 1..11: mol_type = other RNA
cggtgtatggc

SEQ ID NO: 22 - RNA, 11 nt, synthetic construct
  misc_feature 1..11: note = Synthetic: modified Kozak
  source 1..11: mol_type = other RNA
cggtgtatgag

SEQ ID NO: 23 - RNA, 11 nt, synthetic construct
  misc_feature 1..11: note = Synthetic: modified Kozak
  source 1..11: mol_type = other RNA
tagtggatgag

SEQ ID NO: 24 - RNA, 11 nt, synthetic construct
  misc_feature 1..11: note = Synthetic: modified Kozak
  source 1..11: mol_type = other RNA
tattggatgag

SEQ ID NO: 25 - RNA, 11 nt, synthetic construct
  misc_feature 1..11: note = Synthetic: modified Kozak
  source 1..11: mol_type = other RNA
cagtggatgag

SEQ ID NO: 26 - RNA, 11 nt, synthetic construct
  misc_feature 1..11: note = Synthetic: modified Kozak
  source 1..11: mol_type = other RNA
cattggatgag

SEQ ID NO: 27 - RNA, 11 nt, synthetic construct
  misc_feature 1..11: note = Synthetic: modified Kozak
  source 1..11: mol_type = other RNA
tggtggatgag

SEQ ID NO: 28 - RNA, 11 nt, synthetic construct
  misc_feature 1..11: note = Synthetic: modified Kozak
  source 1..11: mol_type = other RNA
aattggatgag

SEQ ID NO: 29 - RNA, 11 nt, synthetic construct
  misc_feature 1..11: note = Synthetic: modified Kozak
  source 1..11: mol_type = other RNA
aagtggatgag

SEQ ID NO: 30 - RNA, 11 nt, synthetic construct
  misc_feature 1..11: note = Synthetic: modified Kozak
  source 1..11: mol_type = other RNA
ccgtggatgag

SEQ ID NO: 31 - RNA, 11 nt, synthetic construct
  misc_feature 1..11: note = Synthetic: modified Kozak
  source 1..11: mol_type = other RNA
gggtggatgag

SEQ ID NO: 32 - DNA, 11 bp, synthetic construct
  misc_feature 1..11: note = Synthetic: modified Kozak
  source 1..11: mol_type = other DNA
cggtgtatggc

SEQ ID NO: 33 - DNA, 11 bp, synthetic construct
  misc_feature 1..11: note = Synthetic: modified Kozak
  source 1..11: mol_type = other DNA
cggtgtatgag

SEQ ID NO: 34 - DNA, 11 bp, synthetic construct
  misc_feature 1..11: note = Synthetic: modified Kozak
  source 1..11: mol_type = other DNA
tagtggatgag

SEQ ID NO: 35 - DNA, 11 bp, synthetic construct
  misc_feature 1..11: note = Synthetic: modified Kozak
  source 1..11: mol_type = other DNA
tattggatgag

SEQ ID NO: 36 - DNA, 11 bp, synthetic construct
  misc_feature 1..11: note = Synthetic: modified Kozak
  source 1..11: mol_type = other DNA
cagtggatgag

SEQ ID NO: 37 - DNA, 11 bp, synthetic construct
  misc_feature 1..11: note = Synthetic: modified Kozak
  source 1..11: mol_type = other DNA
cattggatgag

SEQ ID NO: 38 - DNA, 11 bp, synthetic construct
  misc_feature 1..11: note = Synthetic: modified Kozak
  source 1..11: mol_type = other DNA
tggtggatgag

SEQ ID NO: 39 - DNA, 11 bp, synthetic construct
  misc_feature 1..11: note = Synthetic: modified Kozak
  source 1..11: mol_type = other DNA
aattggatgag

SEQ ID NO: 40 - DNA, 11 bp, synthetic construct
  misc_feature 1..11: note = Synthetic: modified Kozak
  source 1..11: mol_type = other DNA
aagtggatgag

SEQ ID NO: 41 - DNA, 11 bp, synthetic construct
  misc_feature 1..11: note = Synthetic: modified Kozak
  source 1..11: mol_type = other DNA
ccgtggatgag

SEQ ID NO: 42 - DNA, 11 bp, synthetic construct
  misc_feature 1..11: note = Synthetic: modified Kozak
  source 1..11: mol_type = other DNA
gggtggatgag

SEQ ID NO: 43 - DNA, 2211 bp, synthetic construct
  misc_feature 1..2211: note = Synthetic: VP coding region
  source 1..2211: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 44 - DNA, 2217 bp, synthetic construct
  misc_feature 1..2217: note = Synthetic: VP coding region
  source 1..2217: mol_type = other DNA
cggtgtatggctgccgacggttatctacccgattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 45 - DNA, 2217 bp, synthetic construct
  misc_feature 1..2217: note = Synthetic: VP coding region
  source 1..2217: mol_type = other DNA
cggtgtatgagtgccgacggttatctacccgattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 46 - protein, 736 aa, synthetic construct
  REGION 1..736: note = Synthetic: VP coding region
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 47 - protein, 736 aa, synthetic construct
  REGION 1..736: note = Synthetic: VP coding region
MSADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 48-70 - skipped (23 entries)

SEQ ID NO: 71 - protein, 736 aa, synthetic construct
  REGION 1..736: note = Synthetic: VP coding region
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 72 - DNA, 2211 bp, synthetic construct
  misc_feature 1..2211: note = Synthetic: VP coding region
  source 1..2211: mol_type = other DNA
atggctgccgatggttatcttccagattggctcgaggacaacctctctgagggcattcgcgagtggtgggacttgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaaacgtccggtagagcagtcgccacaagagccagactcctcctcgggcatcggcaagacaggccagcagcccgctaaaaagagactcaattttggtcagactggcgactcagagtcagtccccgatccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgatttcaacagattccactgccacttttcaccacgtgactggcagcgactcatcaacaacaattggggattccggcccaagagactcaacttcaaactcttcaacatccaagtcaaggaggtcacgacgaatgatggcgtcacaaccatcgctaataaccttaccagcacggttcaagtcttctcggactcggagtaccagcttccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccttctcagatgctgagaacgggcaacaactttaccttcagctacacctttgaggaagtgcctttccacagcagctacgcgcacagccagagcctggaccggctgatgaatcctctcatcgaccaatacctgtattacctgaacagaactcaaaatcagtccggaagtgcccaaaacaaggacttgctgtttagccgtgggtctccagctggcatgtctgttcagcccaaaaactggctacctggaccctgttatcggcagcagcgcgtttctaaaacaaaaacagacaacaacaacagcaattttacctggactggtgcttcaaaatataacctcaatgggcgtgaatccatcatcaaccctggcactgctatggcctcacacaaagacgacgaagacaagttctttcccatgagcggtgtcatgatttttggaaaagagagcgccggagcttcaaacactgcattggacaatgtcatgattacagacgaagaggaaattaaagccactaaccctgtggccaccgaaagatttgggaccgtggcagtcaatttccagagcagcagcacagaccctgcgaccggagatgtgcatgctatgggagcattacctggcatggtgtggcaagatagagacgtgtacctgcagggtcccatttgggccaaaattcctcacacagatggacactttcacccgtctcctcttatgggcggctttggactcaagaacccgcctcctcagatcctcatcaaaaacacgcctgttcctgcgaatcctccggcggagttttcagctacaaagtttgcttcattcatcacccaatactccacaggacaagtgagtgtggaaattgaatgggagctgcagaaagaaaacagcaagcgctggaatcccgaagtgcagtacacatccaattatgcaaaatctgccaacgttgattttactgtggacaacaatggactttatactgagcctcgccccattggcacccgttaccttacccgtcccctgtaa

SEQ ID NO: 73 - RNA, 11 nt, synthetic construct
  misc_feature 1..11: note = Synthetic: modified Kozak
  source 1..11: mol_type = other RNA
tagtgtatgtc

SEQ ID NO: 74 - RNA, 11 nt, synthetic construct
  misc_feature 1..11: note = Synthetic: modified Kozak
  source 1..11: mol_type = other RNA
tagtttatgtc

SEQ ID NO: 75 - RNA, 11 nt, synthetic construct
  misc_feature 1..11: note = Synthetic: modified Kozak
  source 1..11: mol_type = other RNA
tgttttatgtc

SEQ ID NO: 76 - RNA, 11 nt, synthetic construct
  misc_feature 1..11: note = Synthetic: modified Kozak
  source 1..11: mol_type = other RNA
tcttttatgtc

SEQ ID NO: 77 - RNA, 11 nt, synthetic construct
  misc_feature 1..11: note = Synthetic: modified Kozak
  source 1..11: mol_type = other RNA
tagtgtatggg

SEQ ID NO: 78 - RNA, 11 nt, synthetic construct
  misc_feature 1..11: note = Synthetic: modified Kozak
  source 1..11: mol_type = other RNA
tagtttatggg

SEQ ID NO: 79 - RNA, 11 nt, synthetic construct
  misc_feature 1..11: note = Synthetic: modified Kozak
  source 1..11: mol_type = other RNA
tgttttatggg

SEQ ID NO: 80 - RNA, 11 nt, synthetic construct
  misc_feature 1..11: note = Synthetic: modified Kozak
  source 1..11: mol_type = other RNA
tcttttatggg

SEQ ID NO: 81 - RNA, 11 nt, synthetic construct
  misc_feature 1..11: note = Synthetic: modified Kozak
  source 1..11: mol_type = other RNA
tagtgtatggc

SEQ ID NO: 82 - RNA, 11 nt, synthetic construct
  misc_feature 1..11: note = Synthetic: modified Kozak
  source 1..11: mol_type = other RNA
tagtttatggc

SEQ ID NO: 83 - RNA, 11 nt, synthetic construct
  misc_feature 1..11: note = Synthetic: modified Kozak
  source 1..11: mol_type = other RNA
tgttttatggc

SEQ ID NO: 84 - RNA, 11 nt, synthetic construct
  misc_feature 1..11: note = Synthetic: modified Kozak
  source 1..11: mol_type = other RNA
tcttttatggc

SEQ ID NO: 85 - RNA, 11 nt, synthetic construct
  misc_feature 1..11: note = Synthetic: modified Kozak
  source 1..11: mol_type = other RNA
cattgtatgtc

SEQ ID NO: 86 - RNA, 11 nt, synthetic construct
  misc_feature 1..11: note = Synthetic: modified Kozak
  source 1..11: mol_type = other RNA
tcgtttatgga

SEQ ID NO: 87 - RNA, 11 nt, synthetic construct
  misc_feature 1..11: note = Synthetic: modified Kozak
  source 1..11: mol_type = other RNA
cagtttatggt

SEQ ID NO: 88 - RNA, 11 nt, synthetic construct
  misc_feature 1..11: note = Synthetic: modified Kozak
  source 1..11: mol_type = other RNA
cattgtatggt

SEQ ID NO: 89 - RNA, 11 nt, synthetic construct
  misc_feature 1..11: note = Synthetic: modified Kozak
  source 1..11: mol_type = other RNA
tagtgtatgct

SEQ ID NO: 90 - RNA, 11 nt, synthetic construct
  misc_feature 1..11: note = Synthetic: modified Kozak
  source 1..11: mol_type = other RNA
cattgtatgct

SEQ ID NO: 91 - RNA, 11 nt, synthetic construct
  misc_feature 1..11: note = Synthetic: modified Kozak
  source 1..11: mol_type = other RNA
tcttttatgtc

SEQ ID NO: 92 - RNA, 11 nt, synthetic construct
  misc_feature 1..11: note = Synthetic: modified Kozak
  source 1..11: mol_type = other RNA
tgttttatgtc

SEQ ID NO: 93 - RNA, 11 nt, synthetic construct
  misc_feature 1..11: note = Synthetic: modified Kozak
  source 1..11: mol_type = other RNA
tagtttatgtc

SEQ ID NO: 94 - RNA, 11 nt, synthetic construct
  misc_feature 1..11: note = Synthetic: modified Kozak
  source 1..11: mol_type = other RNA
tagtgtatgtc

SEQ ID NO: 95 - RNA, 13 nt, synthetic construct
  misc_feature 1..13: note = Synthetic: modified Kozak
  source 1..13: mol_type = other RNA
cgacatgaatgtc

SEQ ID NO: 96 - RNA, 13 nt, synthetic construct
  misc_feature 1..13: note = Synthetic: modified Kozak
  source 1..13: mol_type = other RNA
agggatggatgtc

SEQ ID NO: 97 - RNA, 13 nt, synthetic construct
  misc_feature 1..13: note = Synthetic: modified Kozak
  source 1..13: mol_type = other RNA
cttgatggatgtc

SEQ ID NO: 98 - RNA, 13 nt, synthetic construct
  misc_feature 1..13: note = Synthetic: modified Kozak
  source 1..13: mol_type = other RNA
cgtgatgtatgtc

SEQ ID NO: 99 - RNA, 13 nt, synthetic construct
  misc_feature 1..13: note = Synthetic: modified Kozak
  source 1..13: mol_type = other RNA
ggcgatggatgtc

SEQ ID NO: 100 - RNA, 13 nt, synthetic construct
  misc_feature 1..13: note = Synthetic: modified Kozak
  source 1..13: mol_type = other RNA
cgtgatggatgtc

SEQ ID NO: 101 - RNA, 13 nt, synthetic construct
  misc_feature 1..13: note = Synthetic: modified Kozak
  source 1..13: mol_type = other RNA
cgtaatgcatgtc

SEQ ID NO: 102 - RNA, 13 nt, synthetic construct
  misc_feature 1..13: note = Synthetic: modified Kozak
  source 1..13: mol_type = other RNA
gaacatgaatgtc

SEQ ID NO: 103 - RNA, 13 nt, synthetic construct
  misc_feature 1..13: note = Synthetic: modified Kozak
  source 1..13: mol_type = other RNA
ggacatgcatgtc

SEQ ID NO: 104 - RNA, 13 nt, synthetic construct
  misc_feature 1..13: note = Synthetic: modified Kozak
  source 1..13: mol_type = other RNA
aagaatgaatgtc

SEQ ID NO: 105 - RNA, 13 nt, synthetic construct
  misc_feature 1..13: note = Synthetic: modified Kozak
  source 1..13: mol_type = other RNA
cccgatggatgtc

SEQ ID NO: 106 - RNA, 13 nt, synthetic construct
  misc_feature 1..13: note = Synthetic: modified Kozak
  source 1..13: mol_type = other RNA
gccgatgtatgtc

SEQ ID NO: 107 - RNA, 13 nt, synthetic construct
  misc_feature 1..13: note = Synthetic: modified Kozak
  source 1..13: mol_type = other RNA
cgggatgaatgtc

SEQ ID NO: 108 - RNA, 14 nt, synthetic construct
  misc_feature 1..14: note = Synthetic: modified Kozak
  source 1..14: mol_type = other RNA
gaccatgcatggct

SEQ ID NO: 109 - RNA, 13 nt, synthetic construct
  misc_feature 1..13: note = Synthetic: modified Kozak
  source 1..13: mol_type = other RNA
aattatgcatggc

SEQ ID NO: 110 - RNA, 13 nt, synthetic construct
  misc_feature 1..13: note = Synthetic: modified Kozak
  source 1..13: mol_type = other RNA
agacatgtatggc

SEQ ID NO: 111 - RNA, 13 nt, synthetic construct
  misc_feature 1..13: note = Synthetic: modified Kozak
  source 1..13: mol_type = other RNA
aagaatgtatggc

SEQ ID NO: 112 - RNA, 14 nt, synthetic construct
  misc_feature 1..14: note = Synthetic: modified Kozak
  source 1..14: mol_type = other RNA
gaccatgcatggct

SEQ ID NO: 113 - RNA, 13 nt, synthetic construct
  misc_feature 1..13: note = Synthetic: modified Kozak
  source 1..13: mol_type = other RNA
cgcgatggatggc

SEQ ID NO: 114 - RNA, 13 nt, synthetic construct
  misc_feature 1..13: note = Synthetic: modified Kozak
  source 1..13: mol_type = other RNA
agcgatggatggc

SEQ ID NO: 115 - RNA, 13 nt, synthetic construct
  misc_feature 1..13: note = Synthetic: modified Kozak
  source 1..13: mol_type = other RNA
gatcatgcatggc

SEQ ID NO: 116 - RNA, 13 nt, synthetic construct
  misc_feature 1..13: note = Synthetic: modified Kozak
  source 1..13: mol_type = other RNA
cgtaatggatggc

SEQ ID NO: 117 - RNA, 13 nt, synthetic construct
  misc_feature 1..13: note = Synthetic: modified Kozak
  source 1..13: mol_type = other RNA
aacgatgtatggc

SEQ ID NO: 118 - DNA, 11 bp, synthetic construct
  misc_feature 1..11: note = Synthetic: modified Kozak
  source 1..11: mol_type = other DNA
tagtgtatgtc

SEQ ID NO: 119 - DNA, 11 bp, synthetic construct
  misc_feature 1..11: note = Synthetic: modified Kozak
  source 1..11: mol_type = other DNA
tagtttatgtc

SEQ ID NO: 120 - DNA, 11 bp, synthetic construct
  misc_feature 1..11: note = Synthetic: modified Kozak
  source 1..11: mol_type = other DNA
tgttttatgtc

SEQ ID NO: 121 - DNA, 11 bp, synthetic construct
  misc_feature 1..11: note = Synthetic: modified Kozak
  source 1..11: mol_type = other DNA
tcttttatgtc

SEQ ID NO: 122 - DNA, 11 bp, synthetic construct
  misc_feature 1..11: note = Synthetic: modified Kozak
  source 1..11: mol_type = other DNA
tagtgtatggg

SEQ ID NO: 123 - DNA, 11 bp, synthetic construct
  misc_feature 1..11: note = Synthetic: modified Kozak
  source 1..11: mol_type = other DNA
tagtttatggg

SEQ ID NO: 124 - DNA, 11 bp, synthetic construct
  misc_feature 1..11: note = Synthetic: modified Kozak
  source 1..11: mol_type = other DNA
tgttttatggg

SEQ ID NO: 125 - DNA, 11 bp, synthetic construct
  misc_feature 1..11: note = Synthetic: modified Kozak
  source 1..11: mol_type = other DNA
tcttttatggg

SEQ ID NO: 126 - DNA, 11 bp, synthetic construct
  misc_feature 1..11: note = Synthetic: modified Kozak
  source 1..11: mol_type = other DNA
tagtgtatggc

SEQ ID NO: 127 - DNA, 11 bp, synthetic construct
  misc_feature 1..11: note = Synthetic: modified Kozak
  source 1..11: mol_type = other DNA
tagtttatggc

SEQ ID NO: 128 - DNA, 11 bp, synthetic construct
  misc_feature 1..11: note = Synthetic: modified Kozak
  source 1..11: mol_type = other DNA
tgttttatggc

SEQ ID NO: 129 - DNA, 11 bp, synthetic construct
  misc_feature 1..11: note = Synthetic: modified Kozak
  source 1..11: mol_type = other DNA
tcttttatggc

SEQ ID NO: 130 - DNA, 11 bp, synthetic construct
  misc_feature 1..11: note = Synthetic: modified Kozak
  source 1..11: mol_type = other DNA
cattgtatgtc

SEQ ID NO: 131 - DNA, 11 bp, synthetic construct
  misc_feature 1..11: note = Synthetic: modified Kozak
  source 1..11: mol_type = other DNA
tcgtttatgga

SEQ ID NO: 132 - DNA, 11 bp, synthetic construct
  misc_feature 1..11: note = Synthetic: modified Kozak
  source 1..11: mol_type = other DNA
cagtttatggt

SEQ ID NO: 133 - DNA, 11 bp, synthetic construct
  misc_feature 1..11: note = Synthetic: modified Kozak
  source 1..11: mol_type = other DNA
cattgtatggt

SEQ ID NO: 134 - DNA, 11 bp, synthetic construct
  misc_feature 1..11: note = Synthetic: modified Kozak
  source 1..11: mol_type = other DNA
tagtgtatgct

SEQ ID NO: 135 - DNA, 11 bp, synthetic construct
  misc_feature 1..11: note = Synthetic: modified Kozak
  source 1..11: mol_type = other DNA
cattgtatgct

SEQ ID NO: 136 - DNA, 11 bp, synthetic construct
  misc_feature 1..11: note = Synthetic: modified Kozak
  source 1..11: mol_type = other DNA
tcttttatgtc

SEQ ID NO: 137 - DNA, 11 bp, synthetic construct
  misc_feature 1..11: note = Synthetic: modified Kozak
  source 1..11: mol_type = other DNA
tgttttatgtc

SEQ ID NO: 138 - DNA, 11 bp, synthetic construct
  misc_feature 1..11: note = Synthetic: modified Kozak
  source 1..11: mol_type = other DNA
tagtttatgtc

SEQ ID NO: 139 - DNA, 11 bp, synthetic construct
  misc_feature 1..11: note = Synthetic: modified Kozak
  source 1..11: mol_type = other DNA
tagtgtatgtc

SEQ ID NO: 140 - DNA, 13 bp, synthetic construct
  misc_feature 1..13: note = Synthetic: modified Kozak
  source 1..13: mol_type = other DNA
cgacatgaatgtc

SEQ ID NO: 141 - DNA, 13 bp, synthetic construct
  misc_feature 1..13: note = Synthetic: modified Kozak
  source 1..13: mol_type = other DNA
agggatggatgtc

SEQ ID NO: 142 - DNA, 13 bp, synthetic construct
  misc_feature 1..13: note = Synthetic: modified Kozak
  source 1..13: mol_type = other DNA
cttgatggatgtc

SEQ ID NO: 143 - DNA, 13 bp, synthetic construct
  misc_feature 1..13: note = Synthetic: modified Kozak
  source 1..13: mol_type = other DNA
cgtgatgtatgtc

SEQ ID NO: 144 - DNA, 13 bp, synthetic construct
  misc_feature 1..13: note = Synthetic: modified Kozak
  source 1..13: mol_type = other DNA
ggcgatggatgtc

SEQ ID NO: 145 - DNA, 13 bp, synthetic construct
  misc_feature 1..13: note = Synthetic: modified Kozak
  source 1..13: mol_type = other DNA
cgtgatggatgtc

SEQ ID NO: 146 - DNA, 13 bp, synthetic construct
  misc_feature 1..13: note = Synthetic: modified Kozak
  source 1..13: mol_type = other DNA
cgtaatgcatgtc

SEQ ID NO: 147 - DNA, 13 bp, synthetic construct
  misc_feature 1..13: note = Synthetic: modified Kozak
  source 1..13: mol_type = other DNA
gaacatgaatgtc

SEQ ID NO: 148 - DNA, 13 bp, synthetic construct
  misc_feature 1..13: note = Synthetic: modified Kozak
  source 1..13: mol_type = other DNA
ggacatgcatgtc

SEQ ID NO: 149 - DNA, 13 bp, synthetic construct
  misc_feature 1..13: note = Synthetic: modified Kozak
  source 1..13: mol_type = other DNA
aagaatgaatgtc

SEQ ID NO: 150 - DNA, 13 bp, synthetic construct
  misc_feature 1..13: note = Synthetic: modified Kozak
  source 1..13: mol_type = other DNA
cccgatggatgtc

SEQ ID NO: 151 - DNA, 13 bp, synthetic construct
  misc_feature 1..13: note = Synthetic: modified Kozak
  source 1..13: mol_type = other DNA
gccgatgtatgtc

SEQ ID NO: 152 - DNA, 13 bp, synthetic construct
  misc_feature 1..13: note = Synthetic: modified Kozak
  source 1..13: mol_type = other DNA
cgggatgaatgtc

SEQ ID NO: 153 - DNA, 14 bp, synthetic construct
  misc_feature 1..14: note = Synthetic: modified Kozak
  source 1..14: mol_type = other DNA
gaccatgcatggct

SEQ ID NO: 154 - DNA, 13 bp, synthetic construct
  misc_feature 1..13: note = Synthetic: modified Kozak
  source 1..13: mol_type = other DNA
aattatgcatggc

SEQ ID NO: 155 - DNA, 13 bp, synthetic construct
  misc_feature 1..13: note = Synthetic: modified Kozak
  source 1..13: mol_type = other DNA
agacatgtatggc

SEQ ID NO: 156 - DNA, 13 bp, synthetic construct
  misc_feature 1..13: note = Synthetic: modified Kozak
  source 1..13: mol_type = other DNA
aagaatgtatggc

SEQ ID NO: 157 - DNA, 14 bp, synthetic construct
  misc_feature 1..14: note = Synthetic: modified Kozak
  source 1..14: mol_type = other DNA
gaccatgcatggct

SEQ ID NO: 158 - DNA, 13 bp, synthetic construct
  misc_feature 1..13: note = Synthetic: modified Kozak
  source 1..13: mol_type = other DNA
cgcgatggatggc

SEQ ID NO: 159 - DNA, 13 bp, synthetic construct
  misc_feature 1..13: note = Synthetic: modified Kozak
  source 1..13: mol_type = other DNA
agcgatggatggc

SEQ ID NO: 160 - DNA, 13 bp, synthetic construct
  misc_feature 1..13: note = Synthetic: modified Kozak
  source 1..13: mol_type = other DNA
gatcatgcatggc

SEQ ID NO: 161 - DNA, 13 bp, synthetic construct
  misc_feature 1..13: note = Synthetic: modified Kozak
  source 1..13: mol_type = other DNA
cgtaatggatggc

SEQ ID NO: 162 - DNA, 13 bp, synthetic construct
  misc_feature 1..13: note = Synthetic: modified Kozak
  source 1..13: mol_type = other DNA
aacgatgtatggc

SEQ ID NO: 163 - skipped

SEQ ID NO: 164 - DNA, 504 bp, synthetic construct
  misc_feature 1..504: note = Synthetic: ctx promoter
  source 1..504: mol_type = other DNA
tgaaactaacttacaagttggctagtttgttaaaatacgcgctgcgcttgactcgggaatacaaagaaaacattattccacactttgatcacttgactcgattgcgcgatttaatcgacggcttgattaaaagcgaggatgtacaacgttttaatcgcactaatcgcaatgatttaatttcggcttgcttgcaaatcaacgttcggacgtacttgcccaacgccacgatagatttgcgcaaacaacccaactgtatatattttcgaatttgccaatattgccacttggaggccgacgtgccttcgcccgacgatcattcggtgtacagatacttgtgcgtcgcgtgcggcacgccgctggtcatcgaccacccgctcgacgtgttcggccacacggaggaaggcgtcaacgaactgctcgaggtgcagcgagtcaacgcgggcggggagttgtaggcgtcataactatttattaaataagataatttaaaaaatcgccgttaat

SEQ ID NO: 165 - DNA, 29 bp, synthetic construct
  misc_feature 1..29: note = Synthetic: ctx promoter
  source 1..29: mol_type = other DNA
ataagataatttaaaaaatcgccgttaat

SEQ ID NO: 166 - DNA, 65 bp, synthetic construct
  misc_feature 1..65: note = Synthetic: ctx promoter
  source 1..65: mol_type = other DNA
gggcggggagttgtaggcgtcataactatttattaaataagataatttaaaaaatcgccgttaat

SEQ ID NO: 167 - DNA, 92 bp, synthetic construct
  misc_feature 1..92: note = Synthetic: polh promoter
  source 1..92: mol_type = other DNA
atcatggagataattaaaatgataaccatctcgcaaataaataagtattttactgttttcgtaacagttttgtaataaaaaaacctataaat

SEQ ID NO: 168 - protein, 724 aa, synthetic construct
  REGION 1..724: note = Synthetic: VP coding region
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 169 - DNA, 2175 bp, synthetic construct
  misc_feature 1..2175: note = Synthetic: VP coding region
  source 1..2175: mol_type = other DNA
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctctactactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaa

SEQ ID NO: 170-199 - skipped (30 entries)

SEQ ID NO: 200 - DNA, 101 bp, synthetic construct
  misc_feature 1..101: note = Synthetic: p10 promoter
  source 1..101: mol_type = other DNA
gacctttaattcaacccaacacaatatattatagttaaataagaattattatcaaatcatttgtatattaattaaaatactatactgtaaattacatttta

SEQ ID NO: 201 - DNA, 1866 bp, synthetic construct
  misc_feature 1..1866: note = Synthetic: Rep coding region
  source 1..1866: mol_type = other DNA
atggcggggttttacgagattgtgattaaggtccccagcgaccttgacgagcatctgcccggcatttctgacagctttgtgaactgggtggccgagaaggagtgggagttgccgccagattctgacttggatctgaatctgattgagcaggcacccctgaccgtggccgagaagctgcagcgcgactttctgacggagtggcgccgtgtgagtaaggccccggaggcccttttctttgtgcaatttgagaagggagagagctacttccacttacacgtgctcgtggaaaccaccggggtgaaatccttagttttgggacgtttcctgagtcagattcgcgaaaaactgattcagagaatttaccgcgggatcgagccgactttgccaaactggttcgcggtcacaaagaccagaaacggcgccggaggcgggaacaaggtggtggacgagtgctacatccccaattacttgctccccaaaacccagcctgagctccagtgggcgtggactaatttagaacagtatttaagcgcctgtttgaatctcacggagcgtaaacggttggtggcgcagcatctgacgcacgtgtcgcagacgcaggagcagaacaaagagaatcagaatcccaattctgacgcgccggtgatcagatcaaaaacttcagccagatacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 202 - protein, 621 aa, synthetic construct
  REGION 1..621: note = Synthetic: Rep coding region
MAGFYEIVIKVPSDLDEHLPGISDSFVNWVAEKEWELPPDSDLDLNLIEQAPLTVAEKLQRDFLTEWRRVSKAPEALFFVQFEKGESYFHLHVLVETTGVKSLVLGRFLSQIREKLIQRIYRGIEPTLPNWFAVTKTRNGAGGGNKVVDECYIPNYLLPKTQPELQWAWTNLEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 203 - DNA, 1194 bp, synthetic construct
  misc_feature 1..1194: note = Synthetic: Rep coding region
  source 1..1194: mol_type = other DNA
atggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 204 - protein, 397 aa, synthetic construct
  REGION 1..397: note = Synthetic: Rep coding region
MELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 205 - DNA, 2211 bp, synthetic construct
  misc_feature 1..2211: note = Synthetic: VP coding region
  source 1..2211: mol_type = other DNA
acggctgccgacggttatctacccgattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 206-211 - skipped (6 entries)

SEQ ID NO: 212 - DNA, 2211 bp, synthetic construct
  misc_feature 1..2211: note = Synthetic: VP coding region
  source 1..2211: mol_type = other DNA
acggctgccgacggttatctacccgattggctcgaggacaacctctctgagggcattcgcgagtggtgggacttgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaaacgtccggtagagcagtcgccacaagagccagactcctcctcgggcatcggcaagacaggccagcagcccgctaaaaagagactcaattttggtcagactggcgactcagagtcagtccccgatccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgatttcaacagattccactgccacttttcaccacgtgactggcagcgactcatcaacaacaattggggattccggcccaagagactcaacttcaaactcttcaacatccaagtcaaggaggtcacgacgaatgatggcgtcacaaccatcgctaataaccttaccagcacggttcaagtcttctcggactcggagtaccagcttccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccttctcagatgctgagaacgggcaacaactttaccttcagctacacctttgaggaagtgcctttccacagcagctacgcgcacagccagagcctggaccggctgatgaatcctctcatcgaccaatacctgtattacctgaacagaactcaaaatcagtccggaagtgcccaaaacaaggacttgctgtttagccgtgggtctccagctggcatgtctgttcagcccaaaaactggctacctggaccctgttatcggcagcagcgcgtttctaaaacaaaaacagacaacaacaacagcaattttacctggactggtgcttcaaaatataacctcaatgggcgtgaatccatcatcaaccctggcactgctatggcctcacacaaagacgacgaagacaagttctttcccatgagcggtgtcatgatttttggaaaagagagcgccggagcttcaaacactgcattggacaatgtcatgattacagacgaagaggaaattaaagccactaaccctgtggccaccgaaagatttgggaccgtggcagtcaatttccagagcagcagcacagaccctgcgaccggagatgtgcatgctatgggagcattacctggcatggtgtggcaagatagagacgtgtacctgcagggtcccatttgggccaaaattcctcacacagatggacactttcacccgtctcctcttatgggcggctttggactcaagaacccgcctcctcagatcctcatcaaaaacacgcctgttcctgcgaatcctccggcggagttttcagctacaaagtttgcttcattcatcacccaatactccacaggacaagtgagtgtggaaattgaatgggagctgcagaaagaaaacagcaagcgctggaatcccgaagtgcagtacacatccaattatgcaaaatctgccaacgttgattttactgtggacaacaatggactttatactgagcctcgccccattggcacccgttaccttacccgtcccctgtaa

SEQ ID NO: 213 - DNA, 2175 bp, synthetic construct
  misc_feature 1..2175: note = Synthetic: VP coding region
  source 1..2175: mol_type = other DNA
acgagttttgttgatcacccacccgattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggcccggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctcttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaa

SEQ ID NO: 214-216 - skipped (3 entries)

SEQ ID NO: 217 - DNA, 166 bp, synthetic construct
  misc_feature 1..166: note = Synthetic: gp64 promoter
  source 1..166: mol_type = other DNA
aaattatcgcaagataaggcgcacgttgattgggtcacccgagtgtacgttgataaagtcacgtgggcacccaacgcgttgataagcatcggtatataagggcctacagtgttctggtaaatcagttgcactgtgctcttcacaggaacactacaagacctacaag

SEQ ID NO: 218 - protein, 204 aa, synthetic construct
  REGION 1..204: note = Synthetic: AAP protein
METQTQYLTPSLSDSHQQPPLVWELIRWLQAVAHQWQTITRAPTEWVIPREIGIAIPHGWATESSPPAPEPGPCPPTTTTSTNKFPANQEPRTTITTLATAPLGGILTSTDSTATFHHVTGKDSSTTTGDSDPRDSTSSSLTFKSKRSRRMTVRRRLPITLPARFRCLLTRSTSSRTSSARRIKDASRRSQQTSSWCHSMDTSP

SEQ ID NO: 219 - DNA, 648 bp, synthetic construct
  misc_feature 1..648: note = Synthetic: AAP protein
  source 1..648: mol_type = other DNA
atggagacacagacgcagtacctgacgccatcattgtctgatagccatcagcagcctccgcttgtatgggaacttatacgttggctccaggccgtcgcccatcaatggcagacgatcactagggctcctaccgaatgggtaatccccagagagattggtatcgccatcccccatggctgggctaccgaatcatctcctcccgcccctgagccaggtccttgtcctccgacaactacaacttcgacgaataaattcccggctaaccaagagccacgtactaccatcacaactttggcaacagctccactgggtggaatactcacaagcacagactcgactgctacgttccaccatgtgacaggtaaggatagtagtacaacgaccggcgactctgatccaagggactctacgtcatcgtctttgacttttaagtcgaaaagaagccgtcgtatgactgttcgtcgcaggcttcccattacccttcccgctaggttcagatgcctccttacacgcagtaccagttccagaacttcatcggctaggagaataaaggatgcttctagacgcagccaacaaactagttcatggtgccattccatggacactagcccaagcggagactataaggatgatgacgacaagtaataa

SEQ ID NO: 220 - DNA, 92 bp, synthetic construct
  misc_feature 1..92: note = Synthetic: polh promoter
  source 1..92: mol_type = other DNA
atcttggagataattaaattgataaccatctcgcaaataaataagtattttactgttttcgtaacagttttgtaataaaaaaacctataaat

SEQ ID NO: 221 - DNA, 282 bp, synthetic construct
  misc_feature 1..282: note = Synthetic: gp64-polh promoter
  source 1..282: mol_type = other DNA
aaattatcgcaagataaggcgcacgttgattgggtcacccgagtgtacgttgataaagtcacgtgggcacccaacgcgttgataagcatcggtatataagggcctacagtgttctggtaaatcagttgcactgtgctcttcacaggaacactacaagacctacaaggctagcgtatacatcttggagataattaaattgataaccatctcgcaaataaataagtattttactgttttcgtaacagttttgtaataaaaaaacctataaatagatctaccgcc

SEQ ID NO: 222 - RNA, 11 nt, synthetic construct
  misc_feature 1..11: note = Synthetic: modified Kozak
  source 1..11: mol_type = other RNA
aggtgtatggc

SEQ ID NO: 223 - DNA, 11 bp, synthetic construct
  misc_feature 1..11: note = Synthetic: modified Kozak
  source 1..11: mol_type = other DNA
aggtgtatggc

(19) United States
(12) Patent Application Publication
(10) Pub. No.: US 2025/0346920 A1
(43) Pub. Date: Nov. 13, 2025

(54) BACULOVIRUS EXPRESSION SYSTEM

(71) Applicant: Voyager Therapeutics, Inc., Lexington, MA (US)

(72) Inventors: Christopher Cain Lam NGUYEN, Cambridge, MA (US); Jeffrey Morley SLACK, Newton, MA (US); Amanda Udumma IBE-ENWO, Cambridge, MA (US); Peter Slade, Milton, MA (US); Ryan Joseph NISTLER, Boston, MA (US)

(21) Appl. No.: 18/832,471

(22) PCT Filed: Jan. 25, 2023
(86) PCT No.: PCT/US2023/061290

§ 371 Date: Jul. 23, 2024

Related U.S. Application Data
(60) Provisional application No. 63/302,874, filed on Jan. 25, 2022.
(60) Provisional application No. 63/404,879, filed on Sep. 8, 2022.

(51) Int. Cl. C12N 15/86; C07K 14/005

(52) U.S. Cl. CPC C12N 15/86; C07K 14/005; C12N 2710/14044; C12N 2710/14143; C12N 2750/14122; C12N 2750/14143; C12N 2800/80

[symbol]

### RELATED APPLICATIONS

This application claims priority to U.S. Provisional Application No. 63/302,874 filed on Jan. 25, 2022 and U.S. Provisional Application No. 63/404,879 filed on Sep. 8, 2022; the entire contents of each of which are hereby incorporated by reference in their entirety.

### FIELD

The present disclosure describes viral expression constructs (e.g., baculovirus expression constructs) which include a variant viral genome (e.g., a variant baculovirus genome), as well as methods for making the variant viral genome. The viral expression constructs may be used, for example, to produce high levels of a polypeptide or nucleic acid of interest. The viral expression constructs (e.g., baculovirus expression constructs) may be also used as a component of an adeno-associated virus (AAV) production system.

### BACKGROUND

Baculovirus expression vector systems (BEVS) are widely used to produce abundant recombinant proteins in cultured insect cells. This abundance is achieved by expressing the gene of interest (GOI) in recombinant BEVs under control of hyper-expressed polh or p10 promoters. The polh and p10 promoters can account for 24% and 7.5% of total mRNA transcripts in infected insect cells respectively (Chen et al., J Virol 2013; 87:6391-405). Foreign GOI are most commonly inserted into single locations in recombinant BEVs by either homologous recombination in insect cells (e.g., FlashBac®) or by Tn7 auxiliary and/or per os infectivity factor transposition in bacteria (e.g., Bac-to-Bac®). The latter BEV type requires inclusion of a bacterial artificial chromosome in the baculovirus genome. BEVS have been successfully used to produce therapeutics, such as vaccines, e.g., Cervarix™ (HPV vaccine against cervical cancer), FluBlok® (an influenza subunit vaccine), and Covovax™ (SARS-CoV-2 vaccine). Conventional BEVS leave the baculovirus genome in its wild-type form, largely intact. Attempts have been made to improve production yields of recombinant proteins by deleting certain genes, such as viral cathepsin (v-cath) and chitinase (chiA) (e.g., Gilbert et al., PloS One 2018; 13:e0207414). These genes are auxiliary genes involved in baculovirus pathology in caterpillars and are not required for BEV production of foreign proteins in cultured insect cells. Other examples of auxiliary gene include egt and ctx which modulate the physiology of caterpillars during baculovirus infection. Another group of baculovirus genes are per os infectivity factor genes which are involved in the oral transmission of baculoviruses between caterpillars in the environment. The per os infectivity factor genes include p74, p10, polh, and PIFs. It is desirable to inactivate or delete per os infectivity factor and auxiliary genes to improve the safety and efficiency of BEVs for the production of recombinant proteins cultured insect cells. Given the large size of baculovirus genomes (e.g., about 130 kb for AcMNPV) and the scattered locations of auxiliary and per os infectivity factor genes, the many manipulations to the genome that are needed to optimize BEV efficiency and safety are difficult to achieve with conventional recombinant DNA technologies. Thus, there remains a need for improved baculovirus expression systems and methods which allow for efficient, targeted, and multiloci modifications of large baculovirus genomes.

### SUMMARY

Provided herein are viral expression constructs (e.g., baculovirus expression constructs) comprising variant viral genomes (e.g., variant baculovirus genomes), as well as methods for efficient production of the same. Such viral expression constructs (e.g., baculovirus expression constructs) may be used, for example, to produce high levels of a polypeptide or nucleic acid of interest, as well as for production of recombinant adeno-associated virus (AAV) particles in an AAV production system. The viral expression constructs (e.g., baculovirus expression constructs) and methods of production described herein are advantageous over existing systems in that, inter alia, they allow for the efficient synthesis and single-nucleotide level modification of large genomes (>130 kb), which are impractical with existing systems.

In one aspect, provided herein is a baculovirus expression construct comprising at least two subgenomic regions, wherein each subgenomic region comprises: (i) a first unique junction and a second unique junction, wherein the first unique junction is present at the 5′ end of the subgenomic region, and the second unique junction is present at the 3′ end of the subgenomic region; and (ii) a variant baculovirus nucleotide sequence comprising at least 5 fewer functional restriction enzyme sites (e.g., functional naturally occurring restriction enzyme sites), relative to a nucleotide sequence in a reference baculovirus genome, e.g., a nucleotide sequence in a wild-type baculovirus genome. In some embodiments, the functional restriction enzyme sites comprise type II restriction enzyme sites, e.g., type IIS restriction enzyme sites. In some embodiments, the baculovirus expression construct is replication-competent. In some embodiments, the subgenomic region is devoid of recognition sites for one or more selected type IIS restriction enzymes, e.g., BsaI and/or BsmBI. In some embodiments, the subgenomic region is devoid of recognition sites for one or more selected type II restriction enzymes, e.g., AgeI, AvrII, BamHI, NheI, AscI, NotI, or an isoschizomer of any of the aforesaid enzymes. In some embodiments, the reference baculovirus genome is a genome of a baculovirus selected from Autographa californica multiple nucleopolyhedrovirus (AcMNPV) (e.g., an AcMNPV strain E2, C6 or HR3), Bombyx mori nucleopolyhedrovirus (BmNPV), Anticarsia gemmatalis nucleopolyhedrovirus (AgMNPV), Orgyia pseudotsugata nucleopolyhedrovirus (OpMNPV), Thysanoplusia orichalcea nucleopolyhedrovirus (ThorMNPV), or a variant thereof.

In one aspect, provided herein is a baculovirus expression construct comprising at least two subgenomic regions, wherein each subgenomic region comprises: (i) a first unique junction and a second unique junction, wherein the first unique junction is present at the 5′ end of the subgenomic region, and the second unique junction is present at the 3′ end of the subgenomic region; and (ii) a variant baculovirus nucleotide sequence comprising at least 5 fewer functional type IIS restriction enzyme sites, relative to the nucleotide sequence of a wild-type baculovirus genome; wherein the baculovirus expression construct is replication-competent. In some embodiments, the subgenomic region comprises no functional recognition sites for one or more selected type IIS restriction enzymes, e.g., BsaI and/or BsmBI.

In another aspect, provided herein is a plurality of fragments, e.g., subgenomic fragments or subfragments, wherein each fragment comprises: (i) a unique 5′ overhang and a unique 3′ overhang; (ii) a variant baculovirus nucleotide sequence comprising at least 5 fewer functional restriction enzyme sites (e.g., functional naturally occurring restriction enzyme sites), relative to a nucleotide sequence in a reference baculovirus genome, e.g., a nucleotide sequence in a wild-type baculovirus genome. In some embodiments, the functional restriction enzyme sites comprise type II restriction enzyme sites, e.g., type IIS restriction enzyme sites. In some embodiments, the unique 5′ and 3′ overhangs are 2-6 nucleotides in length. In some embodiments, with the exception of recognition sites for producing the unique 5′ and 3′ overhangs, the fragments are devoid of recognition sites for one or more selected type IIS restriction enzymes, e.g., BsaI and/or BsmBI. In some embodiments, the fragments are devoid of recognition sites for one or more selected type II restriction enzymes, e.g., AgeI, AvrII, BamHI, NheI, AscI, NotI, or an isoschizomer thereof.

In another aspect, provided herein is a plurality of fragments, e.g., subgenomic fragments or subfragments, wherein each fragment comprises: (i) a unique 5′ overhang and a unique 3′ overhang; (ii) a variant baculovirus nucleotide sequence comprising at least 5 fewer functional type IIS restriction enzyme sites, relative to the nucleotide sequence of a wild-type baculovirus genome. In some embodiments, the unique 5′ and 3′ overhangs are 2-6 nucleotides in length. In some embodiments, with the exception of recognition sites for producing the unique 5′ and 3′ overhangs, the fragments are devoid of recognition sites for one or more selected type IIS restriction enzymes, e.g., BsaI and/or BsmBI.

In yet another aspect, provided herein is a variant baculovirus genome which comprises at least 5 fewer functional restriction enzyme sites (e.g., functional naturally occurring restriction enzyme sites), relative to a reference baculovirus genome, e.g., a wild-type baculovirus genome. In some embodiments, the functional restriction enzyme sites comprise type II restriction enzyme sites, e.g., type IIS restriction enzyme sites. In some embodiments, the baculovirus expression construct is replication-competent. In some embodiments, the variant baculovirus genome is devoid of recognition sites for one or more selected type IIS restriction enzymes, e.g., BsaI and/or BsmBI. In some embodiments, the variant baculovirus genome is devoid of recognition sites for one or more selected type II restriction enzymes, e.g., AgeI, AvrII, BamHI, NheI, AscI, NotI, or an isoschizomer thereof. In some embodiments, the variant baculovirus genome is 50-200 kb (e.g., 100-180, 120-160, or 130-140 kb) in size.

In yet another aspect, provided herein is a variant baculovirus genome which comprises at least 5 fewer functional type IIS restriction enzyme sites, relative to the nucleotide sequence of a wild-type baculovirus genome, wherein the baculovirus expression construct is replication-competent.

In a further aspect, provided herein is a vector comprising a baculovirus expression construct described herein or a plurality of fragments described herein. In some embodiments, the plurality of fragments is a plurality of subgenomic fragments. In some embodiments, each subgenomic fragment of the plurality is present in a first carrier vector. In some embodiments, the plurality of fragments is a plurality of subfragments. In some embodiments, each subfragment of the plurality is present in a second carrier vector. In some embodiments, the restriction enzyme (e.g., type IIS restriction enzyme) which generates the unique 5′ and 3′ overhangs of a subfragment is a different restriction enzyme (e.g., type IIS restriction enzyme) than that used to generate the unique 5′ and 3′ overhangs of a subgenomic fragment. In some embodiments, the restriction enzyme which generates a subfragment (e.g., a subfragment generated by digesting a carrier vector comprising the subfragment) is a different restriction enzyme than that used to generate a subgenomic fragment (e.g., a subgenomic fragment generated by digesting a carrier vector comprising the subgenomic fragment). In some embodiments, the restriction enzyme used to generate the unique 5′ and 3′ overhangs of a subfragment is BsaI, and the restriction enzyme used to generate the unique 5′ and 3′ overhangs of a subgenomic fragment is BsmBI, or vice versa.

In another aspect, provided herein is a bacterial artificial chromosome (BAC), e.g., mini F replicon, which comprises at least 5 fewer functional restriction enzyme sites (e.g., functional naturally occurring restriction enzyme sites), relative to a reference BAC, e.g., a wild-type BAC. In some embodiments, the functional restriction enzyme sites comprise type II restriction enzyme sites, e.g., type IIS restriction enzyme sites.

In yet another aspect, provided herein is a vector comprising a baculovirus genome or variant thereof, wherein the vector is a BAC, and wherein the BAC comprises at least 5 fewer functional restriction enzyme sites (e.g., functional naturally occurring restriction enzyme sites), relative to a reference BAC, e.g., a wild-type BAC. In some embodiments, the functional restriction enzyme sites comprise type II restriction enzyme sites, e.g., type IIS restriction enzyme sites.

In yet another aspect, provided herein is a cell (e.g., host cell, such as an insect cell) comprising a baculovirus expression construct described herein. The cell can be, e.g., a bacterial cell (e.g., E. coli), a mammalian cell (e.g., HEK293), or an insect cell (e.g., Sf9, Sf21).

In yet another aspect, provided herein is a method of generating a variant baculovirus genome, comprising: (i) providing a plurality of fragments, e.g., subgenomic fragments or subfragments, wherein each fragment comprises: (a) a unique 5′ overhang and 3′ overhang; (b) a variant baculovirus nucleotide sequence comprising at least 5 fewer functional restriction enzyme sites (e.g., functional naturally-occurring sites), relative to a nucleotide sequence in a reference baculovirus genome, e.g., a nucleotide sequence in a wild-type baculovirus genome; (ii) introducing a modification (e.g., insertion, substitution, or deletion), e.g., one or more modifications, into one or more fragments comprising the variant baculovirus nucleotide sequence; and (iii) incubating the plurality of fragments under conditions suitable to form a variant baculovirus genome; thereby generating the variant baculovirus genome. In some embodiments, the functional restriction enzyme sites comprise type II restriction enzyme sites, e.g., type IIS restriction enzyme sites.

In yet another aspect, provided herein is a method of generating a variant baculovirus genome, comprising: (i) providing a plurality of fragments, e.g., subgenomic fragments or subfragments, wherein each fragment comprises: (a) a unique 5′ overhang and 3′ overhang; (b) a variant baculovirus nucleotide sequence comprising at least 5 fewer functional restriction enzyme sites (e.g., functional naturally occurring restriction enzyme sites), relative to a nucleotide sequence in a reference baculovirus genome, e.g., a nucleotide sequence in a wild-type baculovirus genome; wherein one or more fragments of the plurality comprise a modification (e.g., insertion, substitution, or deletion), e.g., one or more modifications, in the variant baculovirus nucleotide sequence; and (iii) incubating the plurality of fragments under conditions suitable to form a variant baculovirus genome; thereby generating the variant baculovirus genome. In some embodiments, the functional restriction enzyme sites comprise type II restriction enzyme sites, e.g., type IIS restriction enzyme sites.

In another aspect, provided herein is a method of producing a plurality of subgenomic fragments capable of assembly into a variant baculovirus genome, as well as a plurality of subgenomic fragments produced by the method, the method comprising: (i) providing a reference, e.g., parental, baculovirus genome; (ii) optionally, identifying one or more sites, e.g., all recognition sites (e.g., functional naturally occurring restriction enzyme sites), recognized by a restriction enzyme, in the reference baculovirus genome, (iii) modifying the one or more recognition sites such that the baculovirus genome comprises at least 5 fewer functional restriction enzyme sites, relative to a nucleotide sequence in a reference baculovirus genome, e.g., a nucleotide sequence in a wild-type baculovirus genome, thereby generating a variant baculovirus genome, (iv) partitioning the primary template into the plurality of subgenomic fragments, wherein each subgenomic fragment of the plurality comprises a unique 5′ overhang and a unique 3′ overhang, and wherein the subgenomic fragments are capable of ordered assembly based on complementarity of the 5′ overhang in one subgenomic fragment with the 3′ overhang in another subgenomic fragment; thereby producing the plurality of subgenomic fragments. In some embodiments, the functional restriction enzyme sites comprise type II restriction enzyme sites, e.g., type IIS restriction enzyme sites.

In another aspect, provided herein is a method of producing a variant baculovirus genome, as well as a variant baculovirus genome made by the method and a baculovirus construct comprising the variant baculovirus genome, the method comprising: (i) providing the plurality of subgenomic fragments, (ii) assembling the plurality of subgenomic fragments into a variant baculovirus genome, and (iii) optionally inserting the variant baculovirus genome into a baculovirus expression construct.

In a further aspect, provided herein is a method of modifying a variant baculovirus genome comprising: (i) providing a plurality of subgenomic fragments described herein, (ii) identifying one or more locations in the baculovirus genome to which one or more modifications (e.g., substitutions, insertions, or deletions), are desired, (iii) selecting the corresponding subgenomic fragment that contains the one or more loci to which one or more modifications are to be introduced, (iv) introducing the one or more modifications into the subgenomic fragment, thereby generating one or more modified subgenomic fragments; (v) incubating the plurality of fragments under conditions suitable to form a variant baculovirus genome by ordered assembly of the plurality of subgenomic fragments, wherein the one or more modified subgenomic fragments replace the non-modified version of the one or more subgenomic fragments within the plurality, thereby obtaining a modified variant baculovirus genome.

In another aspect, provided herein is a baculovirus expression construct or variant baculovirus genome comprising an AAV expression construct and/or AAV payload construct described herein, as well as AAV viral production systems comprising the same.

In another aspect, provided herein is a method of producing a recombinant AAV (rAAV) particle in an AAV viral production cell, as well as rAAV particles produced using the method, wherein the method comprises: (i) providing an AAV viral production system described herein, wherein the baculovirus expression construct or variant baculovirus genome comprises an AAV expression construct which comprises one or more VP-coding regions which comprise one or more nucleotide sequences encoding VP1, VP2 and VP3 capsid proteins; (ii) transfecting the AAV viral production system and/or the baculovirus expression construct or variant baculovirus genome comprising an AAV payload construct comprising a nucleotide sequence encoding a payload into an AAV viral production cell; (iii) exposing the AAV viral production cell to conditions which allow the AAV viral production cell to process the AAV expression construct and the AAV payload construct into rAAV particles; and, optionally, (iv) collecting the rAAV particles from the AAV viral production cell, e.g., an insect cell such as a Sf9 cell or a Sf21cell.

In some embodiments, the variant baculovirus genomes, baculovirus expression vectors, and BACs described herein comprise nucleotide sequences encoding an AAV Rep protein, e.g., Rep40, Rep52, Rep68, Rep78, or a combination thereof. In some embodiments, the variant baculovirus genomes described herein comprise nucleotide sequences encoding an AAV capsid protein, e.g., a VP1 protein, a VP2 protein, a VP3, protein or a combination thereof. In some embodiments, the variant baculovirus genomes described herein comprise nucleotide sequences encoding an AAV1 capsid protein, an AAV2 capsid protein, an AAV3 capsid protein, an AAV4 capsid protein, an AAV5 capsid protein, an AAV6 capsid protein, an AAV8 capsid protein, an AAV9 capsid protein, an AAVrh10 capsid protein, or a variant thereof.

In another aspect, provided herein is a baculovirus expression vector or variant baculovirus genome comprising an AAV expression construct which comprises (i) at least two Rep-coding regions, each comprising a nucleotide sequence encoding a Rep protein independently chosen from Rep52, Rep40, Rep68, or Rep78 protein, e.g., a Rep52 protein and a Rep78 protein; and (ii) a VP-coding region comprising a nucleotide sequence encoding at least one, two, or three VP proteins, chosen from a VP1 protein, a VP2 protein, a VP3 protein, or a combination thereof, wherein the at least two Rep-coding regions each comprise a different nucleotide sequence and/or is present in different location; wherein the baculovirus expression construct comprises at least a portion of a baculovirus genome, e.g., a variant baculovirus genome, comprising a disruption of at least two non-essential genes (e.g., auxiliary and/or per os infectivity factor genes), wherein the at least two non-essential genes are independently chosen from egt, p74 (PIF0), p26, SOD, ChiA, v-cath, p10, polyhedrin, ctx, odv-e56, PIF1, PIF2, PIF3, PIF4, PIF5, Tn7, AcORF-91, AcORF-108, AcORF-52, v-ubi, or p94; optionally wherein the AAV expression construct is stably maintained for at least 5-10 passages, e.g., at least 5, 6, 7, 8, 9, or 10 passages, in a host cell (e.g., an insect cell). In some embodiments, the VP-coding region comprises a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein. In some embodiments, the AAV expression construct comprises a second VP-coding region. In some embodiments, the second VP-coding region comprises a nucleotide sequence encoding primarily a VP1 protein, e.g., at least 50%, 60%, 70%, 80%, 90% or more VP1 protein relative to a VP2 protein or a VP3 protein (e.g., but not a VP2 or a VP3 protein). In some embodiments, the second VP-coding region is operably linked to a ctx promoter. In some embodiments, the AAV expression construct comprises a modified Kozak sequence. In some embodiments, the modified Kozak sequence is present at the 5′ end of the VP-coding region.

In another aspect, provided herein is a baculovirus expression construct or variant baculovirus genome comprising an AAV expression construct which comprises: (i) a Rep-coding region comprising a nucleotide sequence encoding a Rep protein chosen from Rep52, Rep40, Rep68, Rep78 protein, or a combination thereof, e.g., a Rep52 protein and/or a Rep78 protein; and (ii) a VP-coding region comprising a nucleotide sequence encoding at least one, two, or three VP proteins chosen from a VP1 protein, a VP2 protein, a VP3 protein, or a combination thereof, wherein the baculovirus expression construct comprises at least a portion of a baculovirus genome, e.g., a variant baculovirus genome, comprising a disruption of at least two non-essential genes (e.g., auxiliary and/or per os infectivity factor genes), wherein the at least two non-essential genes are independently chosen from egt, p74 (PIF0), p26, SOD, ChiA, v-cath, p10, polyhedrin, ctx, odv-e56, PIF1, PIF2, PIF3, PIF4, PIF5, Tn7, AcORF-91, AcORF-108, AcORF-52, v-ubi, or p94; and wherein the Rep-coding region is operably linked to a first promoter, e.g., a baculovirus early promoter or a baculovirus early-late promoter (e.g., a gp64 promoter), and optionally a second promoter, e.g., a baculovirus later or a baculovirus very late promoter (e.g., a polh promoter), optionally, wherein: (a) the first promoter results in transcription of the Rep-coding region prior to transcription of the VP-coding region; (b) the Rep-coding region is present downstream of a homologous repeat region hr5; and/or (c) the VP-coding region is present in the SOD locus. In some embodiments, the Rep-coding region comprises a nucleotide sequence encoding a Rep78 protein and a Rep52 protein, wherein the nucleotide sequence encoding the Rep52 protein is comprised within the nucleotide sequence encoding the Rep78 protein. In some embodiments, the Rep coding region comprises a single polycistronic ORF encoding a Rep78 protein and a Rep52 protein. In some embodiments, the Rep-coding region is operably linked to a first promoter and/or second promoter, for example, a baculovirus early promoter, baculovirus late promoter, baculovirus early-late promoter, or a baculovirus very late promoter. In some embodiments, the first promoter is a baculovirus early-late promoter and the second promoter is a baculovirus very late promoter. In some embodiments, the first promoter is a gp64 promoter and the second promoter is a polh promoter. In some embodiments, the Rep-coding region is present in the p74 locus. In some embodiments, the AAV expression vector comprises, in 5′ to 3′ order: a first promoter (e.g., a baculovirus early-late promoter such as a gp64 promoter), a second promoter (a baculovirus very late promoter such as a polh promoter), and the Rep-coding region comprising a nucleotide sequence encoding a Rep78 protein and Rep52 protein.

In some embodiments, the variant baculovirus genomes described herein comprise nucleotide sequences encoding a payload, e.g., a therapeutic protein or functional variant thereof; an antibody or antibody fragment; an enzyme; a component of a gene editing system; an RNAi agent (e.g., a dsRNA, siRNA, shRNA, pre-miRNA, pri-miRNA, miRNA, stRNA, lncRNA, piRNA, or snoRNA); or a combination thereof.

In yet another aspect, provided herein are baculoviruses produced using the variant baculovirus genomes described herein, baculovirus expression constructs described herein, plurality of fragments described herein, vectors described herein, BACs described herein, and cells described herein.

In yet another aspect, provided herein are compositions (e.g., pharmaceutical compositions) and kits comprising, e.g., the variant baculovirus genomes described herein, baculovirus expression constructs described herein, plurality of fragments described herein, vectors described herein, BACs described herein, or AAV particles described herein.

Those skilled in the art will recognize or be able to ascertain using no more than routine experimentation, many equivalents to the specific embodiments of the invention described herein. Such equivalents are intended to be encompassed by the following enumerated embodiments.

### Enumerated Embodiments

E1. A baculovirus expression construct comprising at least two subgenomic regions, wherein each subgenomic region comprises:
(i) a first unique junction and a second unique junction, wherein the first unique junction is present at the 5′ end of the subgenomic region, and the second unique junction is present at the 3′ end of the subgenomic region; and
(ii) a variant baculovirus nucleotide sequence comprising at least 5 fewer functional restriction enzyme sites (e.g., functional naturally occurring restriction enzyme sites), e.g., type II restriction enzyme sites (e.g., type IIS restriction enzyme sites), relative to a nucleotide sequence in a reference baculovirus genome, e.g., a nucleotide sequence in a wild-type baculovirus genome;
optionally, wherein the baculovirus expression construct is replication-competent.
   E2. The baculovirus expression construct of embodiment E1, wherein the variant baculovirus nucleotide sequence comprises at least 10, at least 15, at least 20, at least 25, at least 30, at least 35, at least 40, at least 45, at least 50, at least 55, or at least 60 fewer, e.g., 15-60, 15-50, 15-40, 15-30, 15-20, 20-60, 20-50, 20-40, 20-30, 30-60, 30-50, 30-40, 40-60, 40-50, or 50-60 fewer functional restriction enzyme sites (e.g., functional naturally occurring restriction enzyme sites), e.g., type II restriction enzyme sites (e.g., type IIS restriction enzyme sites), relative to a nucleotide sequence in a reference baculovirus genome, e.g., a nucleotide sequence in a wild-type baculovirus genome.
   E3. The baculovirus expression construct of embodiment E1 or E2, wherein the variant baculovirus nucleotide sequence comprises:
(i) at least 10, at least 15, at least 20, at least 25, at least 30, at least 35, at least 40, at least 45, at least 50, at least 55, or at least 60 fewer, e.g., 15-60, 15-50, 15-40, 15-30, 15-20, 20-60, 20-50, 20-40, 20-30, 30-60, 30-50, 30-40, 40-60, 40-50, or 50-60 fewer, functional recognition sites (e.g., functional naturally occurring restriction enzyme sites) of a type II restriction enzyme, e.g., a selected type II restriction enzyme relative to a nucleotide sequence in a reference baculovirus genome, e.g., a nucleotide sequence in a wild-type baculovirus genome; or
(ii) at least 10, at least 15, at least 20, at least 25, at least 30, at least 35, at least 40, at least 45, at least 50, at least 55, or at least 60 fewer, e.g., 15-60, 15-50, 15-40, 15-30, 15-20, 20-60, 20-50, 20-40, 20-30, 30-60, 30-50, 30-40, 40-60, 40-50, or 50-60 fewer, functional recognition sites (e.g., functional naturally occurring restriction enzyme sites) of two or more (e.g., 2-5, 2, 3, 4, or 5) selected type II restriction enzymes relative to a nucleotide sequence in a reference baculovirus genome, e.g., a nucleotide sequence in a wild-type baculovirus genome.
   E4. The baculovirus expression construct of any one of the preceding embodiments, wherein the variant baculovirus nucleotide sequence comprises at least 10, at least 15, at least 20, at least 25, at least 30, at least 35, at least 40, at least 45, at least 50, at least 55, or at least 60 fewer, e.g., 15-60, 15-50, 15-40, 15-30, 15-20, 20-60, 20-50, 20-40, 20-30, 30-60, 30-50, 30-40, 40-60, 40-50, or 50-60 fewer, functional type IIS restriction enzyme sites (e.g., functional naturally occurring type IIS restriction enzyme sites) relative to a nucleotide sequence in a reference baculovirus genome, e.g., a nucleotide sequence in a wild-type baculovirus genome.
   E5. The baculovirus expression construct of any one of the preceding embodiments, wherein the variant baculovirus nucleotide sequence comprises:
(i) at least 10, at least 15, at least 20, at least 25, at least 30, at least 35, at least 40, at least 45, at least 50, at least 55, or at least 60 fewer, e.g., 15-60, 15-50, 15-40, 15-30, 15-20, 20-60, 20-50, 20-40, 20-30, 30-60, 30-50, 30-40, 40-60, 40-50, or 50-60 fewer, functional recognition sites (e.g., functional naturally occurring restriction enzyme sites) of a selected type IIS restriction enzyme relative to a nucleotide sequence in a reference baculovirus genome, e.g., a nucleotide sequence in a wild-type baculovirus genome; or
(ii) at least 10, at least 15, at least 20, at least 25, at least 30, at least 35, at least 40, at least 45, at least 50, at least 55, or at least 60 fewer, e.g., 15-60, 15-50, 15-40, 15-30, 15-20, 20-60, 20-50, 20-40, 20-30, 30-60, 30-50, 30-40, 40-60, 40-50, or 50-60 fewer, functional recognition sites (e.g., functional naturally occurring restriction enzyme sites) of two or more (e.g., 2-5, 2, 3, 4, or 5) selected type IIS restriction enzymes relative to a nucleotide sequence in a reference baculovirus genome, e.g., a nucleotide sequence in a wild-type baculovirus genome.
   E6. The baculovirus expression construct of any one of the preceding embodiments, wherein the variant baculovirus nucleotide sequence comprises:
(i) no functional recognition sites (e.g., functional naturally occurring recognition sites) of two or more (e.g., 2, 3, 4, or 5) selected type II restriction enzymes;
(ii) no functional recognition sites (e.g., functional naturally occurring recognition sites) of a selected type II restriction enzyme; or
(iii) no functional recognition sites (e.g., functional naturally occurring recognition sites) for type II restriction enzymes.
   E7. The baculovirus expression construct of any one of the preceding embodiments, wherein the variant baculovirus nucleotide sequence comprises no functional recognition sites of a selected type IIS restriction enzyme.
   E8. The baculovirus expression construct of any one of the preceding embodiments, wherein the variant baculovirus nucleotide sequence comprises no functional recognition sites of two or more (e.g., 2, 3, 4, or 5) selected type IIS restriction enzymes.
   E9. The baculovirus expression construct of any one of the preceding embodiments, wherein the variant baculovirus nucleotide sequence comprises no functional type IIS restriction enzyme sites.
   E10. The baculovirus expression construct of any one of the preceding embodiments, wherein the variant baculovirus nucleotide sequence is devoid of:
(i) recognition sites (e.g., functional naturally occurring recognition sites) of a selected type II restriction enzyme;
(ii) recognition sites (e.g., functional naturally occurring recognition sites) for 1-5 (e.g., 1-4, 1-3, 1-2, 1, 2, 3, 4, or 5) selected type II restriction enzymes; or
(iii) recognition sites (e.g., functional naturally occurring recognition sites) for type II restriction enzymes.
   E11. The baculovirus expression construct of any one of the preceding embodiments, wherein the variant baculovirus nucleotide sequence is devoid of recognition sites of a selected type IIS restriction enzyme.
   E12. The baculovirus expression construct of any one of the preceding embodiments, wherein the variant baculovirus nucleotide sequence is devoid of recognition sites of two or more (e.g., 2-5, 2, 3, 4, or 5) selected type IIS restriction enzymes.
   E13. The baculovirus expression construct of any one of the preceding embodiments, wherein the variant baculovirus nucleotide sequence is devoid of type IIS restriction enzyme sites.
   E14. The baculovirus expression construct of any one of the preceding embodiments, wherein the restriction enzyme site is recognized by a restriction enzyme that is capable of producing a 5′ overhang upon cleavage by the restriction enzyme.
   E15. The baculovirus expression construct of any one of the preceding embodiments, wherein cleavage by the restriction enzyme results in at least one or at least two (e.g., one or two) base pairs outside the 5′ overhang in its recognition site.
   E16. The baculovirus expression construct of any one of embodiments E1-E13, wherein the restriction enzyme site is recognized by a restriction enzyme that is capable of producing a 3′ overhang upon cleavage by the restriction enzyme.
   E17. The baculovirus expression construct of any one of the preceding embodiments, wherein the restriction enzyme site is recognized by a restriction enzyme that is heat inactivatable.
   E18. The baculovirus expression construct of any one of the preceding embodiments, wherein the restriction enzyme site is recognized by a restriction enzyme that recognizes a stretch of at least 4-8 base pairs, e.g., at least 4 base pairs, at least 5 base pairs, at least 6 base pairs, at least 7 base pairs, or at least 8 base pairs (e.g., 8 base pairs).
   E19. The baculovirus expression construct of any one of the preceding embodiments, wherein the restriction enzyme site is a type II restriction enzyme site.
   E20. The baculovirus expression construct of any one of the preceding embodiments, wherein the restriction enzyme site is recognized by a restriction enzyme that is able to be used in Gibson Assembly™ cloning and ligation method (e.g., a method as described in Gibson et al. “Enzymatic assembly of DNA molecules up to several hundred kilobases,” Nat. Methods, 2009, 6(5):343-5; the contents of which are hereby incorporated by reference in their entirety), e.g., a Gibson Assembly compatible enzyme.
   E21. The baculovirus expression construct of any one of the preceding embodiments, wherein the restriction enzyme site is recognized by a restriction enzyme selected from AcuI, AlwI, AaR1, BaeI, BbsI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, EarI, EciI, Esp3I, FauI, FokI, HgaI, HphI, HpyAV, MbolI, MlyI, MnlI, Nme-AIII, PaqCI, PleI, SapI, SfaNI, AbsI, Acc65I, AgeI, AgsI, ArsI, AscI, AsiGI, AsiSI, Asp718I, AspA2I, AsuNHI, AvrII, Alw44I, AxyI, BamHI, BelI, BcnI, BglII, Bpu1102I, BlnI, BmtI, BsaI, Bse21I, BshTI, BsmBI, Bso31I, BspOI, BspTNI, BstENI, Bsp119I, Bsp120I, Bsu15I, Bsu36I, CciNI, Cfr13I, Csp6I, Cfr9I, CspAI, Ec1136II, Eco31I, Eco53kI, Eco81I, Eco91I, Eco52I, EcoICRI, EcoNI, EcoRI, Esp3I, FseI, Hin6I, HindIII, HpaII, I-CeuI, I-PpoI, I-SceI, KpnI, Kpn2I, LmnI, LpnPI, MauBI, MspI, MluI, NcoI, NdeI, NheI, NotI, PalAI, PI-PspI, PI-SceI, PinAI, Psp124BI, RgaI, RigI, SacI, SalI, SbfI, SdaI, SfaAI, SgfI, SgsI, SmaI, SrfI, Sse8387I, SstI, TaqI, TspMI, XagI, XbaI, XhoI, XmaI, XmaCI, XmaJI, MunI, HinfI, BspTI, Pf123II, VspI, Bsp1407I, Psp1406I, Tru1I, Bpu10I, BcuI, PagI, BshTI, XmaJI, PfoI, SgsI, PteI, KflI, or an isoschizomer of any of the aforesaid restriction enzymes.
   E22. The baculovirus expression construct of any one of the preceding embodiments, wherein the restriction enzyme site is a type IIS restriction enzyme site.
   E23. The baculovirus expression construct of any one of the preceding embodiments, wherein the type IIS restriction enzyme site is recognized by a restriction enzyme selected from AcuI, AlwI, AaR1, BaeI, BbsI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, EarI, EciI, Esp3I, FauI, FokI, HgaI, HphI, HpyAV, MbolI, MlyI, MnlI, Nme-AIII, PaqCI, PleI, SapI, or SfaNI, or an isoschizomer of any of the aforesaid restriction enzymes.
   E24. The baculovirus expression construct of any one of the preceding embodiments, wherein the type IIS restriction enzyme site is a BsmBI restriction enzyme site, a BsaI restriction enzyme site, a PaqCI restriction enzyme site, or a combination thereof.
   E25. The baculovirus expression construct of any one of the preceding embodiments, wherein the type IIS restriction enzyme site is a BsmBI restriction enzyme site.
   E26. The baculovirus expression construct of any one of embodiments E1-E22, E24, or E25, wherein the restriction enzyme site is recognized by a restriction enzyme selected from AbsI, Acc65I, AgeI, AgsI, ArsI, AscI, AsiGI, AsiSI, Asp718I, AspA2I, AsuNHI, AvrII, Alw44I, AxyI, BamHI, BelI, BcnI, BglII, Bpu1102I, BlnI, BmtI, BsaI, Bse21I, BshTI, BsmBI, Bso31I, BspOI, BspTNI, BstENI, Bsp1191, Bsp120I, Bsu15I, Bsu36I, CciNI, Cfr13I, Csp6I, Cfr9I, CspAI, Ec1136II, Eco31I, Eco53kI, Eco81I, Eco91I, Eco52I, EcoICRI, EcoNI, EcoRI, Esp3I, FseI, Hin6I, HindIII, HpaII, I-CeuI, I-PpoI, I-SceI, KpnI, Kpn2I, LmnI, LpnPI, MauBI, MspI, MluI, NcoI, NdeI, NheI, NotI, PalAI, PI-PspI, PI-SceI, PinAI, Psp124BI, RgaI, RigI, SacI, SalI, SbfI, SdaI, SfaAI, SgfI, SgsI, SmaI, SrfI, Sse8387I, SstI, TaqI, TspMI, XagI, XbaI, XhoI, XmaI, XmaCI, XmaJI, MunI, HinfI, BspTI, Pf123II, VspI, Bsp1407I, Psp1406I, Tru1I, Bpu101, BcuI, PagI, BshTI, XmaJI, PfoI, SgsI, PteI, KflI, or an isoschizomer of any of the aforesaid restriction enzymes.
   E27. The baculovirus expression construct of embodiment E26, wherein the restriction enzyme site is recognized by AgeI, AvrII, BamHI, NheI, AscI, NotI, or an isoschizomer of any of the aforesaid restriction enzymes.
   E28. The baculovirus expression construct of embodiment E26 or E27, wherein the restriction enzyme site is recognized by a restriction enzyme selected from AgeI, AvrII, BamHI, NheI, or an isoschizomer of any of the aforesaid restriction enzymes.
   E29. The baculovirus expression construct of embodiment E27, wherein the restriction enzyme site is recognized by a restriction enzyme selected from AscI, NotI, or an isoschizomer of any of the aforesaid restriction enzymes.
   E30. The baculovirus expression construct of any one of the preceding embodiments, wherein the at least one, two, three, four or more of the at least 5 fewer functional type IIS restriction enzyme sites are different type IIS restriction enzyme sites, e.g., a first type IIS restriction enzyme site and a second type IIS restriction enzyme site, optionally wherein:
(i) the first type IIS restriction enzyme site is recognized by a restriction enzyme selected from AcuI, AlwI, AaR1, BaeI, BbsI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, EarI, EciI, Esp3I, FauI, FokI, HgaI, HphI, HpyAV, MbolI, MlyI, MnlI, Nme-AIII, PaqCI, PleI, SapI, or SfaNI, or an isoschizomer of any of the preceding restriction enzymes; and
(ii) the second type IIS restriction enzyme site is recognized by a restriction enzyme selected from AcuI, AlwI, AaR1, BaeI, BbsI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, EarI, EciI, Esp3I, FauI, FokI, HgaI, HphI, HpyAV, MbolI, MlyI, MnlI, Nme-AIII, PaqCI, PleI, SapI, or SfaNI, or an isoschizomer of any of the preceding restriction enzymes.
   E31. The baculovirus expression construct of any one of the preceding embodiments, which comprises 2-20 subgenomic regions (e.g., 16 subgenomic regions).
   E32. The baculovirus expression construct of any one of the preceding embodiments, which comprises at least 3, 4, 5, 6, 7, 8, 9, 10, 11, 12, 13, 14, 15, 16, 17, 18, 19, or 20 subgenomic regions (e.g., 16 subgenomic regions).
   E33. The baculovirus expression construct of any one of the preceding embodiments, wherein one or more of the subgenomic regions is at about 100-25000 bp in length (e.g., about 100-1000 bp, about 100-10000 bp, about 100-20000 bp, about 100-25000 bp, about 1000-10000 bp, about 1000-8000 bp, about 1000-5000 bp, about 1000-2500 bp, about 2500-25000 bp, about 2500-20000 bp, about 2500-15000 bp, about 2500-10000 bp, about 2500-5000 bp, about 5000-25000 bp, about 5000-20000 bp, about 5000-15000 bp, about 5000-10000 bp, about 7500-25000 bp, about 7500-20000, about 7500-15000 bp, about 7500-10000 bp, about 10000-25000 bp, about 10000-20000 bp, about 10000-15000 bp, about 15000-25000 bp, about 15000-20000 bp, about 20000-25000 bp, about 7000-9000 bp, or about 8000 bp in length).
   E34. The baculovirus expression construct of any one of the preceding embodiments, wherein the first unique junction and the second unique junction independently comprise at least 1-50 nucleotides in length, e.g., 2-5 nucleotides (e.g., 4 nucleotides).
   E35. The baculovirus expression construct of any one of the preceding embodiments, wherein the at least two subgenomic regions are formed by a first subgenomic fragment and a second subgenomic fragment.
   E36. The baculovirus expression construct of embodiment E35, wherein each of the first subgenomic fragment and the second subgenomic fragment comprise a unique 5′ overhang and a unique 3′ overhang.
   E37. The baculovirus expression construct of any one of embodiments E31-E36, wherein the 2-20 subgenomic regions are formed by 2-20 subgenomic fragments, e.g., 2, 3, 4, 5, 6, 7, 8, 9, 10, 11, 12, 13, 14, 15, 16, 17, 18, 19, or 20 subgenomic fragments.
   E38. The baculovirus expression construct of any one of embodiments E31-E37, wherein each of the 2-20 subgenomic fragments comprise a unique 5′ overhang and a unique 3′ overhang.
   E39. The baculovirus expression construct of embodiment E39, wherein each of the 2-20 subgenomic fragments comprises a different 5′ overhang and/or a different 3′ overhang relative to the other subgenomic fragments.
   E40. The baculovirus expression construct of any one of the preceding embodiments, wherein the first unique junction comprises the unique 5′ overhang of the first subgenomic fragment and the unique 3′ overhang of the second subgenomic fragment, wherein the unique 5′ overhang of the first subgenomic fragment is complementary (e.g., partially complementary or fully complementary) to the unique 3′ overhang of the second subgenomic fragment.
   E41. The baculovirus expression construct of any one of the preceding embodiments, wherein each of the 2-20 subgenomic fragments comprises a nucleotide sequence at the 5′ end that overlaps with (e.g., is homologous with) the nucleotide sequence at the 3′ end of another subgenomic fragment, wherein the region of overlap is unique to a pair of subgenomic fragments.
   E42. The baculovirus expression construct of any one of embodiments E36-E41, wherein the unique 5′ overhang and the unique 3′ overhang result from cleavage of the subgenomic fragment by a restriction enzyme, e.g., a type II restriction enzyme (e.g., a type IIS restriction enzyme).
   E43. The baculovirus expression construct of any one of embodiments E36-E42, wherein the same restriction enzyme, e.g., the same type II restriction enzyme (e.g., the same type IIS restriction enzyme), is used to generate the unique 5′ overhang and the unique 3′ overhang of each subgenomic fragment.
   E44. The baculovirus expression construct of embodiment E36-E43, wherein the unique 5′ overhang and/or the unique 3′ overhang of the subgenomic fragments independently comprise at least 1-6 nucleotides in length, e.g., 2-5 nucleotides (e.g., 4 nucleotides).
   E45. The baculovirus expression construct of embodiment E36-E44, wherein the unique 5′ overhang and/or the unique 3′ overhang of the subgenomic fragments independently comprise 4 nucleotides.
   E46. The baculovirus expression construct of embodiment E36-E45, wherein the unique 5′ overhang and the unique 3′ overhang of the subgenomic fragments comprise cohesive ends.
   E47. The baculovirus expression construct of embodiment E35-E46, wherein the unique 5′ overhang and unique 3′ overhang of the subgenomic fragments are each independently selected from: ACAA, GGTC, GACC, CCAG, CTGG, CCTT, AAGG, TCAT, ATGA, TCGC, GCGA, AGAG, CTCT, AACT, AGTT, CGGT, ACCG, ATAC, GTAT, GAGT, ACTC, TTCC, GGAA, ATTA, TAAT, TCCT, AGGA, TCTA, TAGA, TGTA, TACA, GATG, CATC, or TTGT, wherein the sequences of the unique 5′ overhang and 3′ overhang in a subgenomic fragment are different.
   E48. The baculovirus expression construct of any one of embodiments E35-E47, wherein one or more of the 2-20 subgenomic fragments are present in a first carrier vector.
   E49. The baculovirus expression construct of any one of embodiments E35-E47, wherein each of the 2-20 subgenomic fragments are present in separate first carrier vectors.
   E50. The baculovirus expression construct of any one of embodiments E35-E48, wherein each of the 2-20 subgenomic fragments are generated from the first carrier vector or the separate first carrier vectors using the same restriction enzyme, e.g., the same type II restriction enzyme, e.g., the same type IIS restriction enzyme.
   E51. The baculovirus expression construct of embodiment E50, wherein the restriction enzyme is capable of producing a 5′ overhang upon cleavage by the restriction enzyme.
   E52. The baculovirus expression construct of embodiment E50 or E51, wherein cleavage by the restriction enzyme results in at least one or at least two (e.g., one or two) base pairs outside the 5′ overhang in its recognition site.
   E53. The baculovirus expression construct of embodiment E50, wherein the restriction enzyme is capable of producing a 3′ overhang upon cleavage by the restriction enzyme.
   E54. The baculovirus expression construct of any one of embodiments E50-E53, wherein the restriction enzyme is heat inactivatable.
   E55. The baculovirus expression construct of any one of embodiments E50-E54, wherein the restriction enzyme recognizes a stretch of at least 4-8 base pairs, e.g., at least 4 base pairs, at least 5 base pairs, at least 6 base pairs, at least 7 base pairs, or at least 8 base pairs (e.g., 8 base pairs).
   E56. The baculovirus expression construct of any one embodiments E50-E55, wherein the restriction enzyme is a type II restriction enzyme.
   E57. The baculovirus expression construct of any one of embodiments E50-E56, wherein the restriction enzyme is a restriction enzyme that is able to be used in a Gibson Assembly™ cloning and ligation method (e.g., a method as described in Gibson et al. “Enzymatic assembly of DNA molecules up to several hundred kilobases,” Nat. Methods, 2009, 6(5):343-5; the contents of which are hereby incorporated by reference in their entirety), e.g., a Gibson Assembly compatible enzyme.
   E58. The baculovirus expression construct of any one of embodiments E50-E57, wherein the restriction enzyme is selected from AcuI, AlwI, AaR1, BaeI, BbsI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, EarI, EciI, Esp3I, FauI, FokI, HgaI, HphI, HpyAV, MbolI, MlyI, MnlI, Nme-AIII, PaqCI, PleI, SapI, SfaNI, AbsI, Acc65I, AgeI, AgsI, ArsI, AscI, AsiGI, AsiSI, Asp718I, AspA2I, AsuNHI, AvrII, Alw44I, AxyI, BamHI, BelI, BcnI, BglII, Bpu1102I, BlnI, BmtI, BsaI, Bse21I, BshTI, BsmBI, Bso31I, BspOI, BspTNI, BstENI, Bsp1191, Bsp120I, Bsu15I, Bsu36I, CciNI, Cfr13I, Csp6I, Cfr9I, CspAI, Ec1136II, Eco31I, Eco53kI, Eco81I, Eco91I, Eco52I, EcoICRI, EcoNI, EcoRI, Esp3I, FseI, Hin6I, HindIII, HpaII, I-CeuI, I-PpoI, I-SceI, KpnI, Kpn2I, LmnI, LpnPI, MauBI, MspI, MluI, NcoI, NdeI, NheI, NotI, PalAI, PI-PspI, PI-SceI, PinAI, Psp124BI, RgaI, RigI, SacI, SalI, SbfI, SdaI, SfaAI, SgfI, SgsI, SmaI, SrfI, Sse8387I, SstI, TaqI, TspMI, XagI, XbaI, XhoI, XmaI, XmaCI, XmaJI, MunI, HinfI, BspTI, Pf123II, VspI, Bsp1407I, Psp1406I, Tru1I, Bpu10I, BcuI, PagI, BshTI, XmaJI, PfoI, SgsI, PteI, KflI, or an isoschizomer of any of the aforesaid restriction enzymes.
   E59. The baculovirus expression construct of any one of embodiments E50-E58, wherein the restriction enzyme is a type IIS restriction enzyme.
   E60. The baculovirus expression construct of any one of embodiments E50-E59, wherein the type IIS restriction enzyme is selected from AcuI, AlwI, AaR1, BaeI, BbsI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, EarI, EciI, Esp3I, FauI, FokI, HgaI, HphI, HpyAV, MbolI, MlyI, MnlI, Nme-AIII, PaqCI, PleI, SapI, or SfaNI, or an isoschizomer of any of the aforesaid restriction enzymes.
   E61. The baculovirus expression construct of any one of embodiments E50-E60, wherein the type IIS restriction enzyme is a BsaI restriction enzyme, a PaqCI restriction enzyme, a BsmBI restriction enzyme, or a combination thereof.
   E62. The baculovirus expression construct of any one of embodiments E50-E61, wherein the type IIS restriction enzyme site is a BsmBI restriction enzyme.
   E63. The baculovirus expression construct of any one of embodiments E50-E62, wherein the restriction enzyme is selected from AbsI, Acc65I, AgeI, AgsI, ArsI, AscI, AsiGI, AsiSI, Asp718I, AspA2I, AsuNHI, AvrII, Alw44I, AxyI, BamHI, BelI, BcnI, BglII, Bpu1102I, BlnI, BmtI, BsaI, Bse21I, BshTI, BsmBI, Bso31I, BspOI, BspTNI, BstENI, Bsp1191, Bsp120I, Bsu15I, Bsu36I, CciNI, Cfr13I, Csp6I, Cfr9I, CspAI, Ec1136II, Eco31I, Eco53kI, Eco81I, Eco91I, Eco52I, EcoICRI, EcoNI, EcoRI, Esp3I, FseI, Hin6I, HindIII, HpaII, I-CeuI, I-PpoI, I-SceI, KpnI, Kpn2I, LmnI, LpnPI, MauBI, MspI, MluI, NcoI, NdeI, NheI, NotI, PalAI, PI-PspI, PI-SceI, PinAI, Psp124BI, RgaI, RigI, SacI, SalI, SbfI, SdaI, SfaAI, SgfI, SgsI, SmaI, SrfI, Sse8387I, SstI, TaqI, TspMI, XagI, XbaI, XhoI, XmaI, XmaCI, XmaJI, MunI, HinfI, BspTI, Pfl23II, VspI, Bsp1407I, Psp1406I, Tru1I, Bpu10I, BcuI, PagI, BshTI, XmaJI, PfoI, SgsI, PteI, KflI, or an isoschizomer of any of the aforesaid restriction enzymes.
   E64. The baculovirus expression construct of any one of E50-E63, wherein the restriction enzyme is AgeI, AvrII, BamHI, NheI, AscI, NotI, or an isoschizomer of any of the aforesaid restriction enzymes.
   E65. The baculovirus expression construct of any one of embodiments E50-E64, wherein the restriction enzyme is AgeI, AvrII, BamHI, NheI, or an isoschizomer of any of the aforesaid restriction enzymes.
   E66. The baculovirus expression construct of any one of embodiments E50-E64, wherein the restriction enzyme is AscI, NotI, or an isoschizomer of any of the aforesaid restriction enzymes.
   E67. The baculovirus expression construct of any one of embodiments E31-E66, wherein one or more of the 2-20 subgenomic fragments comprise a heterologous nucleotide sequence from the variant baculovirus nucleotide sequence.
   E68. The baculovirus expression construct of embodiment E67, wherein the heterologous nucleotide sequence comprises a sequence of interest (e.g., a nucleotide sequence encoding a Rep-coding region, one or more Rep proteins, a VP-coding region, one or more VP proteins, and/or a payload).
   E69. The baculovirus expression construct of embodiment E68, wherein the sequence of interest is operably linked to a first promoter (e.g., a baculovirus early promoter or a baculovirus early-late promoter) and/or a second promoter (e.g., a baculovirus late promoter or a baculovirus very late promoter).
   E70. The baculovirus expression construct of embodiment E67 or E68, wherein the sequence of interest is operably linked to a first promoter (e.g., a baculovirus early promoter or a baculovirus early-late promoter) and a second promoter (e.g., a baculovirus late promoter or a baculovirus very late promoter).
   E71. The baculovirus expression construct of embodiment E69 or E70, wherein the first and/or second promoter is selected from a baculovirus promoter, a viral promoter, an insect viral promoter, a non-insect viral promoter, a vertebrate viral promoter, a chimeric promoter from one or more species including virus and non-virus elements, a synthetic promoter, or a variant thereof.
   E72. The baculovirus expression construct of any one of embodiments E69-E71, wherein the first and/or second promoter chosen from a polh promoter, a p10 promoter, a ctx promoter, a gp64 promoter, an IE promoter, an IE-1 promoter, a p6.9 promoter, a Dmhsp70 promoter, a Hsp70 promoter, a p5 promoter, a p19 promoter, a p35 promoter, a p40 promoter, or a variant, e.g., functional fragment, thereof.
   E73. The baculovirus expression construct of any one of embodiments E69-E72, wherein the first and/or second promoter is selected from a baculovirus early promoter, baculovirus late promoter, baculovirus early-late promoter, or a baculovirus very late promoter.
   E74. The baculovirus expression construct of any one of embodiments E69-E73, wherein first and/or second promoter is a baculovirus early promoter, baculovirus late promoter, or baculovirus early-late promoter.
   E75. The baculovirus expression construct of any one of embodiments E69-E74, wherein first and/or second promoter is a baculovirus early-late promoter (e.g., a gp64 promoter).
   E76. The baculovirus expression construct of any one of embodiments E69-E75, wherein the first or second promoter is a baculovirus very late promoter (e.g., a polh promoter).
   E77. The baculovirus expression construct of any one of embodiments E69-E76, wherein:
(a) the first promoter is an baculovirus early-late promoter and the second promoter is a baculovirus very late promoter,
(b) the first promoter is a baculovirus very late promoter and the second promoter is a baculovirus early-late promoter,
(c) the first promoter is a baculovirus early promoter and the second promoter is a baculovirus early-late promoter,
(d) the first promoter is a baculovirus early-late promoter and the second promoter is a baculovirus early promoter,
(e) the first promoter is a baculovirus early promoter and the second promoter is a baculovirus late promoter,
(f) the first promoter is a baculovirus late promoter and the second promoter is a baculovirus early promoter,
(g) the first promoter is a baculovirus early-late promoter and the second promoter is a baculovirus late promoter,
(h) the first promoter is a baculovirus late promoter and the second promoter is a baculovirus early-late promoter,
(i) the first promoter is a baculovirus late promoter and the second promoter is a baculovirus very-late promoter,
(j) the first promoter is a baculovirus very-late promoter and the second promoter is a baculovirus late promoter,
(k) the first promoter is a baculovirus early promoter and the second promoter is a baculovirus very late promoter,
(l) the first promoter is a baculovirus very late promoter and the second promoter is a baculovirus early promoter,
(m) the first promoter is a baculovirus early promoter and the second promoter is a baculovirus early promoter,
(n) the first promoter is a baculovirus early-late promoter and the second promoter is a baculovirus early-late promoter, or
(o) the first promoter is a baculovirus late promoter and the second promoter is a baculovirus late promoter.
   E78. The baculovirus expression construct of any one of embodiments E69-E77, wherein the first promoter is a baculovirus early-late promoter (e.g., gp64 promoter) and the second promoter is a baculovirus very late promoter (e.g., polh promoter).
   E79. The baculovirus expression construct of any one of embodiments E69-E78, wherein the first promoter is a baculovirus early promoter and the second promoter is a baculovirus late promoter.
   E80. The baculovirus expression construct of any one of embodiments E69-E79, wherein
(a) the baculovirus early promoter is selected from: a lef3 promoter, a dbp promoter, a p35 promoter, an orf82 promoter, an get promoter, an orf81 promoter, an orf122 promoter, a pk-2 promoter, an orf55 promoter, an etl promoter, a hcf-1 promoter, an etm promoter, a lef-2 promoter, a lef-6 promoter, an orf84 promoter, an orf118 promoter, or an orf111 promoter,
(b) the baculovirus early-late promoter is selected from: a lef2 promoter, a orf13 promoter, a orf23 promoter, a pkip promoter, a v-fgf promoter, a pp31 promoter, an odv-e66 promoter, an orf74 promoter, an orf79 promoter, an orf82 promoter, a p15 promoter, a cg30 promoter, a helicase promoter, an he65 promoter, an orf114 promoter, a pk-2 promoter, a gp64 promoter, a gp16 promoter, an alk-exo promoter, a p35 promoter, a me53 promoter, or an ie0 promoter,
(c) the baculovirus late promoter is selected from: a ptpase promoter, an Ac-bro promoter, a ctx promoter, an orf5 promoter, an orf19 promoter, an orf20 promoter, an sod promoter, a HisP promoter, an orf34 promoter, a v-ubi promoter, an orf38 promoter, an orf43 promoter, an orf44 promoter, an orf56 promoter, an orf59 promoter, an orf60 promoter, or an fp-25k promoter, and/or
(d) the baculovirus very late promoter is selected from a p10 promoter or a polh promoter.
   E81. The baculovirus expression construct of any one of embodiments E69-E80, wherein the first and/or second promoter comprises a TATA box motif and/or a CAGT motif.
   E82. The baculovirus expression construct of any one of embodiments E69-E81, wherein the first and/or second promoter comprises a TAAG motif (e.g., an ATAAG nucleotide sequence).
   E83. The baculovirus expression construct of any one of embodiments E69-E82, wherein the first and/or second promoter comprises both a TATA box motif and a TAAG motif.
   E84. The baculovirus expression construct of any one of embodiments E69-E83, wherein the first or second promoter comprises a binding site for VLF-1.
   E85. The baculovirus expression construct of any one of embodiments E69-E84, wherein the first or second promoter is a gp64 promoter (e.g., an OpMNPV gp64 promoter).
   E86. The baculovirus expression construct of any one of embodiments E69-E85, wherein the first or second promoter is a polh promoter (e.g., an OpMNPV polh promoter or an AcMNPV polh promoter).
   E87. The baculovirus expression construct of any one of embodiments E69-E86, wherein the first promoter is a gp64 promoter and the second promoter is a polh promoter, or wherein the first promoter is a polh promoter and the second promoter is a gp64 promoter.
   E88. The baculovirus expression construct of any one of embodiments E69-E87, wherein the Rep-coding region is operably linked to a first promoter which is a baculovirus early-late promoter and a second promoter which is baculovirus very late promoter, e.g., a gp64 promoter and a polh promoter, optionally, wherein the Rep-coding region is present downstream of a homologous repeat region hr5.
   E89. The baculovirus expression construct of any one of embodiments E69-E88, wherein the first promoter is a gp64 promoter and the second promoter is a polh promoter.
   E90. The baculovirus expression construct of embodiment E69-E77 or E80-E86, wherein the first promoter and the second promoter are the same.
   E91. The baculovirus expression construct of embodiment E69-E89, wherein the first promoter and the second promoter are different.
   E92. The baculovirus expression construct of embodiment E90, the first promoter and the second promoter are each a polh promoter.
   E93. The baculovirus expression construct of embodiment E72-E91, wherein the gp64 promoter comprises the nucleotide sequence of SEQ ID NO: 217; a nucleotide sequence at least 70%, 75%, 80%, 85%, 90%, 95%, 96%, 97%, 98%, or 99% identical thereto; a nucleotide sequence comprising at least one, two, three, four, five, six, or seven, but no more than ten different nucleotides relative to SEQ ID NO: 217; or a nucleotide sequence comprising at least one, two, three, four, five, six, or seven, but no more than ten modifications (e.g., substitutions) relative to SEQ ID NO: 217.
   E94. The baculovirus expression construct of embodiment E72-E92, wherein the polh promoter comprises the nucleotide sequence of SEQ ID NO: 167 or 220; a nucleotide sequence at least 70%, 75%, 80%, 85%, 90%, 95%, 96%, 97%, 98%, or 99% identical thereto; a nucleotide sequence comprising at least one, two, three, four, five, six, or seven, but no more than ten different nucleotides relative to SEQ ID NO: 167 or 220; or a nucleotide sequence comprising at least one, two, three, four, five, six, or seven, but no more than ten modifications (e.g., substitutions) relative to SEQ ID NO: 167 or 220.
   E95. The baculovirus expression construct of any one of embodiments E72-E91, wherein the first promoter and the second promoter comprises the nucleotide sequence of SEQ ID NO: 221; a nucleotide sequence at least 70%, 75%, 80%, 85%, 90%, 95%, 96%, 97%, 98%, or 99% identical thereto; a nucleotide sequence comprising at least one, two, three, four, five, six, or seven, but no more than ten different nucleotides relative to SEQ ID NO: 221; or a nucleotide sequence comprising at least one, two, three, four, five, six, or seven, but no more than ten modifications (e.g., substitutions) relative to SEQ ID NO: 221.
   E96. The baculovirus expression construct of any one of embodiments E68-E95, wherein the sequence of interest is a nucleotide sequence encoding a polypeptide of interest or a nucleotide sequence of interest.
   E97. The baculovirus expression construct of any one of embodiments E68-E96, wherein the sequence of interest encodes a therapeutic protein or functional variant thereof, an antibody or antibody fragment; an enzyme; a component of a gene editing system; an RNAi agent (e.g., a dsRNA, siRNA, shRNA, pre-miRNA, pri-miRNA, miRNA, stRNA, lncRNA, piRNA, or snoRNA); or a combination thereof.
   E98. The baculovirus expression construct of any one of embodiments E68-E97, wherein the heterologous nucleotide sequence, e.g., the sequence of interest, comprises one or more adeno-associated viral (AAV) gene encoding regions and/or a payload.
   E99. The baculovirus expression construct of any one of embodiments E68-E98, wherein the heterologous nucleotide sequence is chemically synthesized, a non-templated nucleotide sequence (e.g., non-templated fragment), or generated by a PCR based method.
   E100. The baculovirus expression construct of any one of embodiments E35-E99, wherein the subgenomic fragments, e.g., the 2-20 subgenomic fragments, are capable of ordered assembly based on the complementarity of the 5′ overhang in one subgenomic fragment with the 3′ overhang in another subgenomic fragment to generate a variant baculovirus genome or the baculovirus expression construct.
   E101. The baculovirus expression construct of any one of embodiments E35-E100, wherein the subgenomic fragments, e.g., the 2-20 subgenomic fragments, are capable of ordered assembly based on the overlap in nucleotide sequence at the 5′ end of one subgenomic fragment with the nucleotide sequence at the 3′ end of another subgenomic fragment to generate a variant baculovirus genome or the baculovirus expression construct.
   E102. The baculovirus expression construct of embodiment E100 or E101, wherein the overlap in nucleotide sequence between the subgenomic fragments is 4-50 (e.g., 4-45, 4-40, 4-35, 4-30, 4-25, 4-20, 4-15, 4-10, 10-50, 10-45, 10-40, 10-35, 10-30, 10-25, 10-20, 10-15, 15-50, 15-45, 15-40, 15-35, 15-30, 15-25, 15-20, 20-50, 20-45, 20-40, 20-35, 20-30, 20-25, 25-50, 25-45, 25-40, 25-35, 25-30, 30-50, 30-45, 30-40, 30-35, 35-50, 35-45, 35-40, 40-50, 40-45) contiguous base pairs.
   E103. The baculovirus expression construct of any one of embodiments E100-E102, wherein a 5′ exonuclease creates a single-stranded region of complementarity corresponding to the overlapping nucleotide sequence between subgenomic fragments, producing subgenomic fragments capable of annealing with each other.
   E104. The baculovirus expression construct of any one of embodiments E100-E103, wherein any gaps between the annealed subgenomic fragments are filled in by a DNA polymerase.
   E105. The baculovirus expression construct of any one of embodiments E35-E104, wherein at least two of the subgenomic fragments are covalently linked, e.g., using a ligase (e.g., a T4 ligase), for example using Golden Gate Assembly or Gibson Assembly™, such that at least two subgenomic regions are formed and/or to generate a variant baculovirus genome or the baculovirus expression construct, optionally wherein the subgenomic fragments are ligated in a single step to generate a variant baculovirus genome or the baculovirus expression construct.
   E106. The baculovirus expression construct of any one of embodiments E37-E105, wherein the 2-20 subgenomic fragments are covalently linked, e.g., using a ligase (e.g., a T4 ligase), for example using Golden Gate Assembly or Gibson Assembly™, such that 2-20 subgenomic regions are formed and/or to generate a variant baculovirus genome or the baculovirus expression construct, optionally wherein the subgenomic fragments (e.g., 16 subgenomic fragments) are ligated in a single step to generate a variant baculovirus genome or the baculovirus expression construct.
   E107. The baculovirus expression construct of any one of embodiments E35-E106, wherein at least two of the subgenomic fragments comprises one or more subfragments.
   E108. The baculovirus expression construct of any one of embodiments E37-E107, wherein the 2-20 subgenomic fragments comprises one or more subfragments.
   E109. The baculovirus expression construct of embodiments E37-E108, wherein the 2, 3, 4, 5, 6, 7, 8, 9, 10, 11, 12, 13, 14, 15, 16, 17, 18, 19, or 20 subgenomic regions (e.g., 16 subgenomic regions) comprise one or more subfragments.
   E110. The baculovirus expression construct of any one of embodiments E35-E109, wherein each subgenomic fragment comprises at least 1-20, e.g., 10-20, 12-18, 2, 3, 4, 5, 6, 7, 8, 9, 10, 11, 12, 13, 14, 15, 16, 17, 18, 19, or 20 (e.g., 16) subfragments.
   E111. The baculovirus expression construct of any one of embodiments E37-E110, wherein the 2-20 subgenomic fragments comprises 2-300, e.g., 2-250, 2-200, 2-150, 2-100, 2-50, 2-20, 2-10, 10-300, 10-250, 10-200, 10-150, 10-100, 10-50, 10-20, 15-300, 15-250, 15-200, 15-150, 15-100, 15-50, 15-20, 25-300, 25-250, 25-200, 25-150, 25-100, 25-50, 50-300, 50-250, 50-200, 50-150, 50-100, 100-300, 100-250, 100-200, 100-150, 150-300, 150-250, 150-200, 200-300, 200-250, or 250-300 (e.g., 10-50, 20-40, 32, 200-300, 220-280, 240-260, 256) subfragments.
   E112. The baculovirus expression construct of embodiment E107-E111, wherein each of the one or more subfragments comprises a unique 5′ overhang and a unique 3′ overhang.
   E113. The baculovirus expression construct of embodiment E112, wherein each of the one or more subfragments comprises a different 5′ overhang and/or a different 3′ overhang relative to the other subfragments.
   E114. The baculovirus expression construct of any one of embodiments E107-E113, wherein the one or more subfragments is about 50-1000 bp, e.g., about 50-900, 50-800, 50-700, 50-600, 50-500, 50-400, 50-300, 50-200, 50-100, 100-900, 100-800, 100-700, 100-600, 100-500, 100-400, 100-300, 100-200, 200-1000, 200-900, 200-800, 200-700, 200-600, 200-500, 200-400, 200-300, 400-1000, 400-900, 400-800, 400-700, 400-600, 400-500, 600-1000, 600-900, 600-800, 600-700, 800-1000, 800-900, 900-1000 bp (e.g., 100-800 bp, about 250-750 bp, about 400-600 bp, or about 500 bp) in length.
   E115. The baculovirus expression construct of any one of embodiments E112-E114, wherein the unique 5′ overhang of a first subfragment is complementary (e.g., partially complementary or fully complementary) to the unique 3′ overhang of the second subfragment.
   E116. The baculovirus expression construct of any one of embodiments E112-E115, wherein the unique 5′ overhang and the unique 3′ overhang of the subfragment result from cleavage of the subfragment by a restriction enzyme, e.g., a type IIS restriction enzyme.
   E117. The baculovirus expression construct of any one of embodiments E116, wherein the restriction enzyme, e.g., a type IIS restriction enzyme, is different from the restriction enzyme used to generate the subgenomic fragments.
   E118. The baculovirus expression construct of any one of embodiments E117, wherein the same restriction enzyme, e.g., the same type IIS restriction enzyme, is used to generate the unique 5′ overhang and the unique 3′ overhang of each subfragment, wherein the same restriction enzyme is different from the restriction enzyme used to generate the subgenomic fragments.
   E119. The baculovirus expression construct of any one of embodiments E112-E118, wherein the unique 5′ overhang and the unique 3′ overhang of the subfragments independently comprise at least 1-6 nucleotides in length, e.g., 2-5 nucleotides (e.g., 4 nucleotides).
   E120. The baculovirus expression construct of any one of embodiments E112-E119, wherein the unique 5′ overhang and the unique 3′ overhang of the subfragments independently comprise 4 nucleotides.
   E121. The baculovirus expression construct of any one of embodiments E112-E120, wherein the unique 5′ overhang and the unique 3′ overhang of the subfragments comprise cohesive ends.
   E122. The baculovirus expression construct of any one of embodiments E112-E121, wherein the unique 5′ overhang and unique 3′ overhang of the subfragments are each independently selected from: ACAA, GGTC, GACC, CCAG, CTGG, CCTT, AAGG, TCAT, ATGA, TCGC, GCGA, AGAG, CTCT, AACT, AGTT, CGGT, ACCG, ATAC, GTAT, GAGT, ACTC, TTCC, GGAA, ATTA, TAAT, TCCT, AGGA, TCTA, TAGA, TGTA, TACA, GATG, CATC, or TTGT, wherein the sequences of the unique 5′ overhang and 3′ overhang in a subfragment are different.
   E123. The baculovirus expression construct of any one of embodiments E107-E122, wherein each of the one or more subfragments comprise a region of overlap in nucleotide sequence at the 5′ end with the nucleotide sequence at the 3′ end of another of the one or more subfragments.
   E124. The baculovirus expression construct of embodiment E123, wherein the overlap in nucleotide sequence between the subfragments is 4-50 (e.g., 4-45, 4-40, 4-35, 4-30, 4-25, 4-20, 4-15, 4-10, 10-50, 10-45, 10-40, 10-35, 10-30, 10-25, 10-20, 10-15, 15-50, 15-45, 15-40, 15-35, 15-30, 15-25, 15-20, 20-50, 20-45, 20-40, 20-35, 20-30, 20-25, 25-50, 25-45, 25-40, 25-35, 25-30, 30-50, 30-45, 30-40, 30-35, 35-50, 35-45, 35-40, 40-50, 40-45) contiguous base pairs.
   E125. The baculovirus expression construct of any one of embodiments E107-E124, wherein one or more of the subfragments are present in a second carrier vector.
   E126. The baculovirus expression construct of any one of embodiments E107-E125, wherein each of one or more subfragments are present in separate second carrier vectors.
   E127. The baculovirus expression construct of embodiment any one of embodiments E107-E126, wherein each of the one or more subfragments are generated from the second carrier vector or the separate second carrier vectors using the same restriction enzyme, e.g., the same type II restriction enzyme, e.g., the same type IIS restriction enzyme, wherein the same restriction enzyme is different from the restriction enzyme used to generate the subgenomic fragments.
   E128. The baculovirus expression construct of embodiment E127, wherein the restriction enzyme has one, two, three, four, or all of the following properties:
(i) is capable of producing a 5′ overhang upon cleavage by the restriction enzyme;
(ii) cleavage by the restriction enzyme results in at least one or at least two (e.g., one or two) base pairs outside the 5′ overhang in its recognition site;
(iii) is capable of producing a 3′ overhang upon cleavage by the restriction enzyme;
(iv) is heat inactivatable;
(v) recognizes a stretch of at least 4-8 base pairs, e.g., at least 4 base pairs, at least 5 base pairs, at least 6 base pairs, at least 7 base pairs, or at least 8 base pairs (e.g., 8 base pairs).
   E129. The baculovirus expression construct of embodiment E127 or E128, wherein the restriction enzyme is a type II restriction enzyme.
   E130. The baculovirus expression construct of any one of embodiments E127-E129, wherein the restriction enzyme is a restriction enzyme that is able to be used in Gibson Assembly™ cloning and ligation method (e.g., a method as described in Gibson et al. “Enzymatic assembly of DNA molecules up to several hundred kilobases,” Nat. Methods, 2009, 6(5):343-5; the contents of which are hereby incorporated by reference in their entirety), e.g., a Gibson Assembly compatible enzyme.
   E131. The baculovirus expression construct of any one of embodiments E127-E130, wherein the restriction enzyme is selected from AcuI, AlwI, AaR1, BaeI, BbsI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, EarI, EciI, Esp3I, FauI, FokI, HgaI, HphI, HpyAV, MbolI, MlyI, MnlI, Nme-AIII, PaqCI, PleI, SapI, SfaNI, AbsI, Acc65I, AgeI, AgsI, ArsI, AscI, AsiGI, AsiSI, Asp718I, AspA2I, AsuNHI, AvrII, Alw44I, AxyI, BamHI, BelI, BcnI, BglII, Bpu1102I, BlnI, BmtI, BsaI, Bse21I, BshTI, BsmBI, Bso31I, BspOI, BspTNI, BstENI, Bsp119I, Bsp120I, Bsu15I, Bsu36I, CciNI, Cfr13I, Csp6I, Cfr9I, CspAI, Ec1136II, Eco31I, Eco53kI, Eco81I, Eco91I, Eco52I, EcoICRI, EcoNI, EcoRI, Esp3I, FseI, Hin6I, HindIII, HpaII, I-CeuI, I-PpoI, I-SceI, KpnI, Kpn2I, LmnI, LpnPI, MauBI, MspI, MluI, NcoI, NdeI, NheI, NotI, PalAI, PI-PspI, PI-SceI, PinAI, Psp124BI, RgaI, RigI, SacI, SalI, SbfI, SdaI, SfaAI, SgfI, SgsI, SmaI, SrfI, Sse8387I, SstI, TaqI, TspMI, XagI, XbaI, XhoI, XmaI, XmaCI, XmaJI, MunI, HinfI, BspTI, Pf123II, VspI, Bsp1407I, Psp1406I, Tru1I, Bpu10I, BcuI, PagI, BshTI, XmaJI, PfoI, SgsI, PteI, KflI, or an isoschizomer of any of the aforesaid restriction enzymes
   E132. The baculovirus expression construct of any one of embodiments E127-E131, wherein the restriction enzyme is a type IIS restriction enzyme.
   E133. The baculovirus expression construct of any one of embodiments E127-E132, wherein the type IIS restriction enzyme is selected from AcuI, AlwI, AaR1, BaeI, BbsI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, EarI, EciI, Esp3I, FauI, FokI, HgaI, HphI, HpyAV, MbolI, MlyI, MnlI, Nme-AIII, PaqCI, PleI, SapI, or SfaNI, or an isoschizomer of any of the aforesaid restriction enzymes.
   E134. The baculovirus expression construct of any one of embodiments E127-E133, wherein the type IIS restriction enzyme is a BsaI restriction enzyme, a BsmBI restriction enzyme, a PaqCI restriction enzyme, or a combination thereof.
   E135. The baculovirus expression construct of any one of embodiments E127-E134, wherein the type IIS restriction enzyme is a BsaI restriction enzyme.
   E136. The baculovirus expression construct of any one of embodiments E127-E135, wherein the restriction enzyme is selected from AbsI, Acc65I, AgeI, AgsI, ArsI, AscI, AsiGI, AsiSI, Asp718I, AspA2I, AsuNHI, AvrII, Alw44I, AxyI, BamHI, BelI, BcnI, BglII, Bpu1102I, BlnI, BmtI, BsaI, Bse21I, BshTI, BsmBI, Bso31I, BspOI, BspTNI, BstENI, Bsp119I, Bsp120I, Bsu15I, Bsu36I, CciNI, Cfr13I, Csp6I, Cfr9I, CspAI, Ec1136II, Eco31I, Eco53kI, Eco81I, Eco91I, Eco52I, EcoICRI, EcoNI, EcoRI, Esp3I, FseI, Hin6I, HindIII, HpaII, I-CeuI, I-PpoI, I-SceI, KpnI, Kpn2I, LmnI, LpnPI, MauBI, MspI, MluI, NcoI, NdeI, NheI, NotI, PalAI, PI-PspI, PI-SceI, PinAI, Psp124BI, RgaI, RigI, SacI, SalI, SbfI, SdaI, SfaAI, SgfI, SgsI, SmaI, SrfI, Sse8387I, SstI, TaqI, TspMI, XagI, XbaI, XhoI, XmaI, XmaCI, XmaJI, MunI, HinfI, BspTI, Pf123II, VspI, Bsp1407I, Psp1406I, Tru1I, Bpu10I, BcuI, PagI, BshTI, XmaJI, PfoI, SgsI, PteI, KflI, or an isoschizomer of any of the aforesaid restriction enzymes.
   E137. The baculovirus expression construct of any one of embodiments E127-E136, wherein the restriction enzyme is AgeI, AvrII, BamHI, NheI, AscI, NotI, or an isoschizomer of any of the aforesaid restriction enzymes.
   E138. The baculovirus expression construct of any one of embodiments E127-E137, wherein the restriction enzyme is selected from AgeI, AvrII, BamHI, NheI, or an isoschizomer of any of the aforesaid restriction enzymes.
   E139. The baculovirus expression construct of any one of embodiments E127-E138, wherein the restriction enzyme is selected from AscI, NotI, or an isoschizomer of any of the aforesaid restriction enzymes.
   E140. The baculovirus expression construct of any one of the preceding embodiments, wherein the restriction enzyme is not EcoRI or wherein the restriction enzyme site is not EcoRI.
   E141. The baculovirus expression construct of any one of embodiments E107-E140, wherein one or more of subfragments comprise a heterologous nucleotide sequence from the variant baculovirus nucleotide sequence.
   E142. The baculovirus expression construct of embodiment E141, wherein the heterologous nucleotide sequence comprises a sequence of interest.
   E143. The baculovirus expression construct of embodiment E142, wherein the sequence of interest is a nucleotide sequence encoding a polypeptide of interest or a nucleotide sequence of interest.
   E144. The baculovirus expression construct of embodiment E141 or E142, wherein the sequence of interest encodes a therapeutic protein or functional variant thereof, an antibody or antibody fragment; an enzyme; a component of a gene editing system; an RNAi agent (e.g., a dsRNA, siRNA, shRNA, pre-miRNA, pri-miRNA, miRNA, stRNA, lncRNA, piRNA, or snoRNA); or a combination thereof.
   E145. The baculovirus expression construct of any one of embodiments E141-E144, wherein the heterologous nucleotide sequence, e.g., the sequence of interest, comprises one or more adeno-associated viral (AAV) gene encoding regions and/or a payload.
   E146. The baculovirus expression construct of any one of embodiments E141-E145, wherein the heterologous nucleotide sequence is chemically synthesized, a non-templated nucleotide sequence (e.g., non-templated fragment), or generated by a PCR based assay.
   E147. The baculovirus expression construct of any one of embodiments E107-E146, wherein one or more subfragments are capable of ordered assembly based on the complementarity of the 5′ overhang in one subfragment with the 3′ overhang in another subfragment to generate the subgenomic fragments and/or the baculovirus expression construct.
   E148. The baculovirus expression construct of any one of embodiments E107-E147, wherein the subfragments are capable of ordered assembly based on the overlap in nucleotide sequence at the 5′ end of one subfragment with the nucleotide sequence at the 3′ end of another subfragment to generate the subgenomic fragments and/or the baculovirus expression construct.
   E149. The baculovirus expression construct of embodiment E148, wherein the overlap in nucleotide sequence between the subfragments is 4-50 (e.g., 4-45, 4-40, 4-35, 4-30, 4-25, 4-20, 4-15, 4-10, 10-50, 10-45, 10-40, 10-35, 10-30, 10-25, 10-20, 10-15, 15-50, 15-45, 15-40, 15-35, 15-30, 15-25, 15-20, 20-50, 20-45, 20-40, 20-35, 20-30, 20-25, 25-50, 25-45, 25-40, 25-35, 25-30, 30-50, 30-45, 30-40, 30-35, 35-50, 35-45, 35-40, 40-50, 40-45) contiguous base pairs.
   E150. The baculovirus expression construct of embodiment E148 or E149, wherein a 5′ exonuclease creates a single-stranded region of complementarity corresponding to the overlapping sequence between subfragments, producing subfragments capable of annealing with each other.
   E151. The baculovirus expression construct of embodiment E150, wherein any gaps between the annealed subfragments are filled in by a DNA polymerase.
   E152. The baculovirus expression construct of embodiment any one of embodiments E107-E151, wherein the one or more subfragments are covalently linked, e.g., using a ligase (e.g., a T4 ligase), for example using Golden Gate Assembly or Gibson assembly, such one or more subgenomic regions are formed and/or to generate the baculovirus expression construct, optionally wherein the subfragments are ligated in a single step to generate one or more subgenomic fragments (or subgenomic regions), a variant baculovirus genome or the baculovirus expression construct.
   E153. The baculovirus expression construct of any one of embodiments E107-E152, wherein one or more of the subfragments comprises a variant baculovirus nucleotide sequence comprising at least 10 fewer functional type IIS restriction enzyme sites, relative to a nucleotide sequence in a reference baculovirus genome, e.g., a nucleotide sequence in a wild-type baculovirus genome.
   E154. The baculovirus expression construct of any one of embodiments E35-E153, wherein the variant baculovirus nucleotide sequence of one or more of the subgenomic fragments comprises a modification, e.g., an insertion, deletion, or substitution.
   E155. The baculovirus expression construct of any one of embodiments E35-E154, wherein the variant baculovirus nucleotide sequence of one or more of the subfragments comprises a modification, e.g., an insertion, deletion, or substitution.
   E156. The baculovirus expression construct of embodiment E154 or E155, wherein the modification, e.g., an insertion, deletion, substitution, or mutation (e.g., frame-shift mutation), is present in a non-essential gene (e.g., auxiliary and/or per os infectivity factor gene) or a regulatory region of a non-essential gene (e.g., promoter modification).
   E157. The baculovirus expression construct of embodiment E156, wherein the non-essential gene is selected from one, two, three, or more (e.g., 1, 2, 3, 4, 5, 6, 7, 8, 9, 10, 11, 12, 13, 14, 15, 16, 17, 18, 19, 20, or 21) of egt, p74 (PIF0), p26, SOD, ChiA, v-cath, p10, polyhedrin, ctx, odv-e56, PIF1, PIF2, PIF3, PIF4, PIF5, Tn7, AcORF-91, AcORF-108, AcORF-52, v-ubi, or p94.
   E158. The baculovirus expression construct of embodiment E157, which comprises a modification, e.g., an insertion, deletion, substitution, or mutation (e.g., frame-shift mutation) in:
(i) v-cath and egt;
(ii) v-cath, egt, and SOD;
(iii) chiA, v-cath, egt, p26, p10, and p74;
(iv) chiA, v-cath, egt, p26, p10, p74, and SOD; or
(v) chiA, v-cath, egt, p26, p10, p74, SOD, AcORF-91, and AcORF-108.
   E159. The baculovirus expression construct of any one of embodiments E154-E158, wherein the modification comprises a deletion of a chiA gene, a v-cath gene, a p26 gene, a p10 gene, and/or a p74 gene, or a portion thereof.
   E160. The baculovirus expression construct of any one of embodiments E156-E159, wherein the modification comprises an insertion of a heterologous sequence in the non-essential gene or adjacent region.
   E161. The baculovirus expression construct of any one of embodiments E156-E160, wherein the modification comprises one or more mutations in the non-essential gene or adjacent region.
   E162. The baculovirus expression construct of any one of embodiments E156-E161, wherein the non-essential genes are present near (e.g., downstream or upstream) of a homologous repeat region 5 (hr5).
   E163. The baculovirus expression construct of any one of embodiments E154-E162, wherein the modification, e.g., an insertion, deletion, substitution, or mutation (e.g., frame-shift mutation), results in inactivation of a non-essential gene (e.g., auxiliary and/or per os infectivity factor gene) or a regulatory region of a non-essential gene (e.g., promoter modification or insertion of heterologous DNA adjacent to non-essential gene).
   E164. The baculovirus expression construct of any one of embodiments E35-E163, wherein the variant baculovirus nucleotide sequence of one or more of the subgenomic fragments comprises a disruption, e.g., a mutation (e.g., frameshift mutation), a deletion, an insertion, or inactivation, of a non-essential gene (e.g., auxiliary and/or per os infectivity factor gene) or a regulatory region of a non-essential gene (e.g., promoter modification or insertion of heterologous DNA adjacent to non-essential gene).
   E165. The baculovirus expression construct of any one of embodiments E107-163, wherein the variant baculovirus nucleotide sequence of one or more of the subfragments comprises a disruption, e.g., a mutation (e.g., frameshift mutation), a deletion, an insertion (e.g., insertion of heterologous DNA adjacent to non-essential gene), or inactivation, of a non-essential gene (e.g., auxiliary and/or per os infectivity factor gene) or a regulatory region of a non-essential gene (e.g., promoter modification or insertion of heterologous DNA adjacent to non-essential gene).
   E166. The baculovirus expression construct of any one of embodiments E154-E165, wherein the modification is introduced by chemical synthesis or PCR, e.g., PCR-based site-directed mutagenesis.
   E167. The baculovirus expression construct of any one of embodiments E1-E106, which comprises a baculovirus genome or a portion thereof.
   E168. The baculovirus expression construct of embodiment E167, wherein the baculovirus genome or portion thereof is at least 10 kb-140 kb in length, e.g., at least 10 kb, 20 kb, 30 kb, 40 kb, 50 kb, 60 kb, 70 kb, 80 kb, 90 kb, 100 kb, 110 kb, 120 kb, 130 kb, or 140 kb.
   E169. The baculovirus expression construct of embodiment E167 or E168, wherein the baculovirus genome comprises a nucleotide sequence or a portion thereof from a baculovirus genome selected from Autographa californica multiple nucleopolyhedrovirus (AcMNPV) (e.g., an AcMNPV strain E2, C6 or HR3), Bombyx mori nucleopolyhedrovirus (BmNPV), Anticarsia gemmatalis nucleopolyhedrovirus (AgMNPV), Orgyia pseudotsugata nucleopolyhedrovirus (OpMNPV), Thysanoplusia orichalcea nucleopolyhedrovirus (ThorMNPV), or a variant thereof.
   E170. The baculovirus expression construct of any one of embodiments E167-E169, wherein the baculovirus genome is a wild-type baculovirus genome or a modified baculovirus genome, e.g., a baculovirus genome having a deletion in at least one non-essential gene (e.g., auxiliary and/or per os infectivity factor gene), e.g., having a deletion in a polyhedrin (polh) locus.
   E171. The baculovirus expression construct of any one of embodiments E167-E170 wherein the baculovirus genome is a bMON14272 baculovirus genome.
   E172. The baculovirus expression construct of any one of embodiments E35-E171, wherein one or more of the subgenomic fragments encodes an AAV Rep protein, e.g., Rep40, Rep52, Rep68, Rep78, or a combination thereof.
   E173. The baculovirus expression construct of any one of embodiments E107-E172, wherein one or more of the subfragments encodes an AAV Rep protein, e.g., Rep40, Rep52, Rep68, Rep78, or a combination thereof.
   E174. The baculovirus expression construct of any one of embodiments E35-E173, wherein one or more of the subgenomic fragments or subfragments encodes a Rep78 protein and/or a Rep52 protein.
   E175. The baculovirus expression construct of any one of embodiments E35-E174, wherein one or more of the subgenomic fragments or subfragments encodes an AAV capsid protein, e.g., a VP1 protein, a VP2 protein, a VP3, protein or a combination thereof.
   E176. The baculovirus expression construct of any one of embodiments E35-E175, wherein one or more of the subgenomic fragments or subfragments encodes an AAV1 capsid protein, an AAV2 capsid protein, an AAV3 capsid protein, an AAV4 capsid protein, an AAV5 capsid protein, an AAV6 capsid protein, an AAV8 capsid protein, an AAV9 capsid protein, an AAVrh10 capsid protein, or a variant thereof.
   E177. The baculovirus expression construct of any one of embodiments E35-E176, wherein one or more of the subgenomic fragments or subfragments encodes an AAV5 capsid protein or variant thereof, or an AAV9 capsid protein or variant thereof.
   E178. The baculovirus expression construct of any one of embodiments E35-E177, wherein one or more of the subgenomic fragments or subfragments encodes a payload.
   E179. The baculovirus expression construct of any one of embodiments E178, wherein the encoded payload is selected from a therapeutic protein or functional variant thereof; an antibody or antibody fragment; an enzyme; a component of a gene editing system; an RNAi agent (e.g., a dsRNA, siRNA, shRNA, pre-miRNA, pri-miRNA, miRNA, stRNA, lncRNA, piRNA, or snoRNA); or a combination thereof.
   E180. The baculovirus expression construct of any one of embodiments E1-E179, which is capable of producing at least 70% (e.g., at least 80%, at least 90%, at least 100%, at least 110%, at least 120%, 70-200%, 70-150%, 70-100%, 80-200%, 80-150%, 80-100%, 90-200%, 90-150%, 90-100%, 100-200%, 100-150%, 100-125%, 100-110%, 125-200%, 150-200%, or 175-200%) of the baculovirus produced by a reference baculovirus genome (e.g., a wild-type baculovirus genome).
   E181. A plurality of fragments, e.g., subgenomic fragments or subfragments, wherein each fragment comprises:
(i) a unique 5′ overhang and a unique 3′ overhang; and
(ii) a variant baculovirus nucleotide sequence comprising at least 5 fewer functional restriction enzyme sites (e.g., functional naturally occurring restriction enzyme sites), e.g., type II restriction enzyme sites (e.g., type IIS restriction enzyme sites), relative to a nucleotide sequence in a reference baculovirus genome, e.g., a nucleotide sequence in a wild-type baculovirus genome,
wherein each fragment is double stranded except at the unique 5′ overhang and the unique 3′ overhang, which are single stranded.
   E182. The plurality of fragments of embodiment E181, wherein the variant baculovirus nucleotide sequence comprises at least 10, 15, 20, 25, 30, 35, 40, 45, 50, 55, or 60 fewer, e.g., 15-60, 15-50, 15-40, 15-30, 15-20, 20-60, 20-50, 20-40, 20-30, 30-60, 30-50, 30-40, 40-60, 40-50, or 50-60 fewer, functional restriction enzyme sites (e.g., functional naturally occurring restriction enzyme sites), e.g., type II restriction enzyme sites (e.g., type IIS restriction enzyme sites), relative to a nucleotide sequence in a reference baculovirus genome, e.g., a nucleotide sequence in a wild-type baculovirus genome.
   E183. The plurality of fragments of embodiment E181 or E182, wherein the variant baculovirus nucleotide sequence comprises:
(i) at least 10, at least 15, at least 20, at least 25, at least 30, at least 35, at least 40, at least 45, at least 50, at least 55, or at least 60 fewer, e.g., 15-60, 15-50, 15-40, 15-30, 15-20, 20-60, 20-50, 20-40, 20-30, 30-60, 30-50, 30-40, 40-60, 40-50, or 50-60 fewer, functional recognition sites (e.g., functional naturally occurring recognition sites) of a type II restriction enzyme, e.g., a selected type II restriction enzyme relative to a nucleotide sequence in a reference baculovirus genome, e.g., a nucleotide sequence in a wild-type baculovirus genome; or
(ii) at least 10, at least 15, at least 20, at least 25, at least 30, at least 35, at least 40, at least 45, at least 50, at least 55, or at least 60 fewer, e.g., 15-60, 15-50, 15-40, 15-30, 15-20, 20-60, 20-50, 20-40, 20-30, 30-60, 30-50, 30-40, 40-60, 40-50, or 50-60 fewer, functional recognition sites (e.g., functional naturally occurring recognition sites) of two or more (e.g., 2-5, 2, 3, 4, or 5) selected type II restriction enzymes relative to a nucleotide sequence in a reference baculovirus genome, e.g., a nucleotide sequence in a wild-type baculovirus genome.
   E184. The plurality of fragments of any one of embodiments E181-E183, wherein the variant baculovirus nucleotide sequence comprises at least 10, at least 15, at least 20, at least 25, at least 30, at least 35, at least 40, at least 45, at least 50, at least 55, or at least 60 fewer, e.g., 15-60, 15-50, 15-40, 15-30, 15-20, 20-60, 20-50, 20-40, 20-30, 30-60, 30-50, 30-40, 40-60, 40-50, or 50-60 fewer, functional type IIS restriction enzyme sites (e.g., functional naturally occurring type IIS restriction enzyme sites) relative to a nucleotide sequence in a reference baculovirus genome, e.g., a nucleotide sequence in a wild-type baculovirus genome.
   E185. The plurality of fragments of any one of embodiments E181-E184, wherein the variant baculovirus nucleotide sequence comprises:
(i) at least 10, at least 15, at least 20, at least 25, at least 30, at least 35, at least 40, at least 45, at least 50, at least 55, or at least 60 fewer, e.g., 15-60, 15-50, 15-40, 15-30, 15-20, 20-60, 20-50, 20-40, 20-30, 30-60, 30-50, 30-40, 40-60, 40-50, or 50-60 fewer, functional recognition sites (e.g., functional naturally occurring recognition sites) of a selected type IIS restriction enzyme relative to a nucleotide sequence in a reference baculovirus genome, e.g., a nucleotide sequence in a wild-type baculovirus genome; or
(ii) at least 10, at least 15, at least 20, at least 25, at least 30, at least 35, at least 40, at least 45, at least 50, at least 55, or at least 60 fewer, e.g., 15-60, 15-50, 15-40, 15-30, 15-20, 20-60, 20-50, 20-40, 20-30, 30-60, 30-50, 30-40, 40-60, 40-50, or 50-60 fewer, functional recognition sites (e.g., functional naturally occurring recognition sites) of two or more (e.g., 2-5, 2, 3, 4, or 5) selected type IIS restriction enzymes relative to a nucleotide sequence in a reference baculovirus genome, e.g., a nucleotide sequence in a wild-type baculovirus genome.
   E186. The plurality of fragments of any one of embodiments E181-E185 wherein the variant baculovirus nucleotide sequence comprises:
(i) no functional recognition sites (e.g., functional naturally occurring recognition sites) of two or more (e.g., 2, 3, 4, or 5) selected type II restriction enzymes;
(ii) no functional recognition sites (e.g., functional naturally occurring recognition sites) of a selected type of type II restriction enzyme; or
(iii) no functional recognition sites (e.g., functional naturally occurring recognition sites) for type II restriction enzymes.
   E187. The plurality of fragments of any one of embodiments E181-E186, wherein the variant baculovirus nucleotide sequence comprises no functional recognition sites (e.g., functional naturally occurring recognition sites) of a selected type IIS restriction enzyme.
   E188. The plurality of fragments of any one of embodiments E181-E187, wherein the variant baculovirus nucleotide sequence comprises no functional recognition sites (e.g., functional naturally occurring recognition sites) of two or more (e.g., 2, 3, 4, or 5) selected type IIS restriction enzymes.
   E189. The plurality of fragments of any one of embodiments E181-E188, wherein the variant baculovirus nucleotide sequence comprises no functional recognition sites (e.g., functional naturally occurring recognition sites) for type IIS restriction enzymes.
   E190. The plurality of fragments of any one of embodiments E181-E189, wherein the variant baculovirus nucleotide sequence is devoid of:
(i) recognition sites (e.g., functional naturally occurring recognition sites) of a selected type II restriction enzyme
(ii) recognition sites (e.g., functional naturally occurring recognition sites) for 1-5 (e.g., 1-4, 1-3, 1-2, 1, 2, 3, 4, or 5) selected type II restriction enzymes; or
(iii) recognition sites (e.g., functional naturally occurring recognition sites) for type II restriction enzymes.
   E191. The plurality of fragments of any one of embodiments E181-E190, wherein the type IIS restriction enzyme sites are recognition sites for a selected type IIS restriction enzyme.
   E192. The plurality of fragments of any one of embodiments E181-E191, wherein the type IIS restriction enzyme sites are recognition sites for two or more (e.g., 2, 3, 4, or 5) selected type IIS restriction enzymes.
   E193. The plurality of fragments of any one of embodiments E181-E192, wherein the variant baculovirus nucleotide sequence is devoid of recognition sites of a selected type IIS restriction enzyme.
   E194. The plurality of fragments of any one of embodiments E181-E193, wherein the variant baculovirus nucleotide sequence is devoid of recognition sites of two or more (e.g., 2-5, 2, 3, 4, or 5) selected type IIS restriction enzymes.
   E195. The plurality of fragments of any one of embodiments E181-E194, wherein the variant baculovirus nucleotide sequence is devoid of type IIS restriction enzyme sites.
   E196. The plurality of fragments of any one of embodiments E181-E195, wherein the restriction enzyme site is recognized by a restriction enzyme having one, two, three, four or all of the following properties:
(i) is capable of producing a 5′ overhang upon cleavage by the restriction enzyme;
(ii) cleavage by the restriction enzyme results in at least one or at least two (e.g., one or two) base pairs outside the 5′ overhang in its recognition site;
(iii) is capable of producing a 3′ overhang upon cleavage by the restriction enzyme; (iv) is heat inactivatable; and/or
(v) recognizes a stretch of at least 4-8 base pairs, e.g., at least 4 base pairs, at least 5 base pairs, at least 6 base pairs, at least 7 base pairs, or at least 8 base pairs (e.g., 8 base pairs).
   E197. The plurality of fragments of any one of embodiments E181-E196, wherein the restriction enzyme site is a type II restriction enzyme site.
   E198. The plurality of fragments of any one of embodiments E181-E197, wherein the restriction enzyme site is recognized by a restriction enzyme that is able to be used in Gibson Assembly™ cloning and ligation method (e.g., a method as described in Gibson et al. “Enzymatic assembly of DNA molecules up to several hundred kilobases,” Nat. Methods, 2009, 6(5):343-5; the contents of which are hereby incorporated by reference in their entirety), e.g., a Gibson Assembly compatible enzyme.
   E199. The plurality of fragments of any one of embodiments E181-E198, wherein the restriction enzyme site is recognized by a restriction enzyme selected from AcuI, AlwI, AaRI, BaeI, BbsI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, EarI, EciI, Esp3I, FauI, FokI, HgaI, HphI, HpyAV, MbolI, MlyI, MnlI, Nme-AIII, PaqCI, PleI, SapI, SfaNI, AbsI, Acc65I, AgeI, AgsI, ArsI, AscI, AsiGI, AsiSI, Asp718I, AspA2I, AsuNHI, AvrII, Alw44I, AxyI, BamHI, BelI, BcnI, BglII, Bpu1102I, BlnI, BmtI, BsaI, Bse21I, BshTI, BsmBI, Bso31I, BspOI, BspTNI, BstENI, Bsp119I, Bsp120I, Bsu15I, Bsu36I, CciNI, Cfr13I, Csp6I, Cfr9I, CspAI, Ec1136II, Eco31I, Eco53kI, Eco81I, Eco91I, Eco52I, EcoICRI, EcoNI, EcoRI, Esp3I, FseI, Hin6I, HindIII, HpaII, I-CeuI, I-PpoI, I-SceI, KpnI, Kpn2I, LmnI, LpnPI, MauBI, MspI, MluI, NcoI, NdeI, NheI, NotI, PalAI, PI-PspI, PI-SceI, PinAI, Psp124BI, RgaI, RigI, SacI, SalI, SbfI, SdaI, SfaAI, SgfI, SgsI, SmaI, SrfI, Sse8387I, SstI, TaqI, TspMI, XagI, XbaI, XhoI, XmaI, XmaCI, XmaJI, MunI, HinfI, BspTI, Pf123II, VspI, Bsp1407I, Psp1406I, Tru1I, Bpu101, BcuI, PagI, BshTI, XmaJI, PfoI, SgsI, PteI, KflI, or an isoschizomer of any of the aforesaid restriction enzymes.
   E200. The plurality of fragments of any one of embodiments E181-E199, wherein the restriction enzyme site is a type IIS restriction enzyme site.
   E201. The plurality of fragments of any one of embodiments E181-E200, wherein the type IIS restriction enzyme site is recognized by a restriction enzyme selected from AcuI, AlwI, AaRI, BaeI, BbsI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, EarI, EciI, Esp3I, FauI, FokI, HgaI, HphI, HpyAV, MbolI, MlyI, MnlI, Nme-AIII, PaqCI, PleI, SapI, or SfaNI, or an isoschizomer of any of the aforesaid restriction enzymes.
   E202. The plurality of fragments of any one of embodiments E181-E201, wherein the type IIS restriction enzyme sites are a BsmBI restriction enzyme site, a BsaI restriction enzyme site, a PaqCI restriction enzyme site, or a combination thereof.
   E203. The plurality of fragments of any one of embodiments E181-E202, wherein the type IIS restriction enzyme site is a BsmBI restriction enzyme site.
   E204. The plurality of fragments of any one of embodiments E181-E203, wherein the restriction enzyme site is recognized by a restriction enzyme selected from AbsI, Acc65I, AgeI, AgsI, ArsI, AscI, AsiGI, AsiSI, Asp718I, AspA2I, AsuNHI, AvrII, Alw44I, AxyI, BamHI, BelI, BcnI, BglII, Bpu1102I, BlnI, BmtI, BsaI, Bse21I, BshTI, BsmBI, Bso31I, BspOI, BspTNI, BstENI, Bsp1191, Bsp120I, Bsu15I, Bsu36I, CciNI, Cfr13I, Csp6I, Cfr9I, CspAI, Ec1136II, Eco31I, Eco53kI, Eco81I, Eco91I, Eco52I, EcoICRI, EcoNI, EcoRI, Esp3I, FseI, Hin6I, HindIII, HpaII, I-CeuI, I-PpoI, I-SceI, KpnI, Kpn2I, LmnI, LpnPI, MauBI, MspI, MluI, NcoI, NdeI, NheI, NotI, PalAI, PI-PspI, PI-SceI, PinAI, Psp124BI, RgaI, RigI, SacI, SalI, SbfI, SdaI, SfaAI, SgfI, SgsI, SmaI, SrfI, Sse8387I, SstI, TaqI, TspMI, XagI, XbaI, XhoI, XmaI, XmaCI, XmaJI, MunI, HinfI, BspTI, Pf123II, VspI, Bsp1407I, Psp1406I, Trull, Bpu10I, BcuI, PagI, BshTI, XmaJI, PfoI, SgsI, PteI, KflI, or an isoschizomer of any of the aforesaid restriction enzymes.
   E205. The plurality of fragments of any one of embodiments E181-E204, wherein the restriction enzyme site is recognized by AgeI, AvrII, BamHI, NheI, AscI, NotI, or an isoschizomer of any of the aforesaid restriction enzymes.
   E206. The plurality of fragments of any one of embodiments E181-E205, wherein the restriction enzyme site is recognized by a restriction enzyme selected from AgeI, AvrII, BamHI, NheI, or an isoschizomer of any of the aforesaid restriction enzymes.
   E207. The plurality of fragments of any one of embodiments E181-E205, wherein the restriction enzyme site is recognized by a restriction enzyme selected from AscI, NotI, or an isoschizomer of any of the aforesaid restriction enzymes.
   E208. The plurality of fragments of any one of embodiments E181-E207, wherein the at least one, two, three, four or more of the at least 5 fewer functional type IIS restriction enzyme sites are different type IIS restriction enzyme sites, e.g., a first type IIS restriction enzyme site and a second type IIS restriction enzyme site, optionally wherein:
(i) the first type IIS restriction enzyme site is recognized by a restriction enzyme selected from AcuI, AlwI, AaR1, BaeI, BbsI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, EarI, EciI, Esp3I, FauI, FokI, HgaI, HphI, HpyAV, MbolI, MlyI, MnlI, Nme-AIII, PaqCI, PleI, SapI, or SfaNI, or an isoschizomer of any of the preceding restriction enzymes; and
(ii) the second type IIS restriction enzyme site is recognized by a restriction enzyme selected from AcuI, AlwI, AaR1, BaeI, BbsI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, EarI, EciI, Esp3I, FauI, FokI, HgaI, HphI, HpyAV, MbolI, MlyI, MnlI, Nme-AIII, PaqCI, PleI, SapI, or SfaNI, or an isoschizomer of any of the preceding restriction enzymes.
   E209. The plurality of fragments of any one of embodiments E181-E208, wherein each fragment comprises a different 5′ overhang and/or a 3′ overhang relative to the other fragments of the plurality.
   E210. The plurality of fragments of any one of embodiments E181-E209, wherein each fragment is capable of ordered assembly based on complementarity of the unique 5′ overhang in one fragment with the unique 3′ overhang in another fragment.
   E211. The plurality of fragments of any one of embodiments E181-E210, wherein the unique 5′ overhang of one fragment of the plurality is complementary to the unique 3′ overhang of another fragment of the plurality.
   E212. The plurality of fragments of any one of embodiments E181-E211, wherein the unique 5′ overhang is partially complementary or fully complementary to the unique 3′ overhang.
   E213. The plurality of fragments of any one of embodiments E181-E212, wherein each fragment of the plurality comprises a nucleotide sequence at the 5′ end that overlaps with (e.g., is homologous with) the nucleotide sequence at the 3′ end of another fragment of the plurality, wherein the region of overlap is unique to a pair of fragments.
   E214. The plurality of fragments of any one of embodiments E181-E213, wherein fragments of the plurality are capable of ordered assembly based on the overlap in nucleotide sequence at the 5′ end of one fragment (e.g., subgenomic fragment or subfragment) with the nucleotide sequence at the 3′ end of another fragment (e.g., subgenomic fragment or subfragment) to generate a variant baculovirus genome or the baculovirus expression construct.
   E215. The baculovirus expression construct of any one of embodiments E181-E214, wherein the overlap in nucleotide sequence between the fragments is 4-50 (e.g., 4-45, 4-40, 4-35, 4-30, 4-25, 4-20, 4-15, 4-10, 10-50, 10-45, 10-40, 10-35, 10-30, 10-25, 10-20, 10-15, 15-50, 15-45, 15-40, 15-35, 15-30, 15-25, 15-20, 20-50, 20-45, 20-40, 20-35, 20-30, 20-25, 25-50, 25-45, 25-40, 25-35, 25-30, 30-50, 30-45, 30-40, 30-35, 35-50, 35-45, 35-40, 40-50, 40-45) contiguous base pairs.
   E216. The plurality of fragments of any one of embodiments E181-E215, wherein the unique 5′ overhang and unique 3′ overhang result from cleavage of the fragment by a restriction enzyme, e.g., a type II restriction enzyme (e.g., a type IIS restriction enzyme).
   E217. The plurality of fragments of any one of embodiments E181-E216, wherein the unique 5′ and 3′ overhang each independently comprise 1-6 nucleotides in length, e.g., 2-5 nucleotides (e.g., 4 nucleotides).
   E218. The plurality of fragments of any one of embodiments E181-E217, wherein the unique 5′ and 3′ overhang each independently comprise 4 nucleotides.
   E219. The plurality of fragments of any one of embodiments E181-E218, wherein the unique 5′ overhang and unique 3′ overhang of the fragments are each independently selected from: ACAA, GGTC, GACC, CCAG, CTGG, CCTT, AAGG, TCAT, ATGA, TCGC, GCGA, AGAG, CTCT, AACT, AGTT, CGGT, ACCG, ATAC, GTAT, GAGT, ACTC, TTCC, GGAA, ATTA, TAAT, TCCT, AGGA, TCTA, TAGA, TGTA, TACA, GATG, CATC, or TTGT, wherein the sequences of the unique 5′ overhang and 3′ overhang in a fragment are different.
   E220. The plurality of fragments of any one of embodiments E181-E219, wherein:
(i) the subgenomic fragments result from cleavage with a first restriction enzyme, e.g., a first type II restriction enzyme (e.g., a first type IIS restriction enzyme); and/or
(ii) the subfragments result from cleavage with a second restriction enzyme, e.g., a second type II restriction enzyme (e.g., a second type IIS restriction enzyme);
wherein the first restriction enzyme, e.g., a first type II restriction enzyme (e.g., a first type IIS restriction enzyme) is different from the second restriction enzyme, e.g., a second type II restriction enzyme (e.g., a second type IIS restriction enzyme).
   E221. The plurality of fragments of any one of embodiments E181-E220, wherein each fragment, e.g., subfragment, of the plurality is between 50-1000 bp, e.g., about 50-900, 50-800, 50-700, 50-600, 50-500, 50-400, 50-300, 50-200, 50-100, 100-900, 100-800, 100-700, 100-600, 100-500, 100-400, 100-300, 100-200, 200-1000, 200-900, 200-800, 200-700, 200-600, 200-500, 200-400, 200-300, 400-1000, 400-900, 400-800, 400-700, 400-600, 400-500, 600-1000, 600-900, 600-800, 600-700, 800-1000, 800-900, or 900-1000 bp (e.g., 100-800 bp, about 250-750 bp, about 400-600 bp, or about 500 bp) in length.
   E222. The plurality of fragments of any one of embodiments E181-E221, wherein each fragment, e.g., subgenomic fragment, of the plurality is between 100-25000 bp in length (e.g., about 100-1000 bp, about 100-10000 bp, about 100-20000 bp, about 100-25000 bp, about 1000-10000 bp, about 1000-8000 bp, about 1000-5000 bp, about 1000-2500 bp, about 2500-25000 bp, about 2500-20000 bp, about 2500-15000 bp, about 2500-10000 bp, about 2500-5000 bp, about 5000-25000 bp, about 5000-20000 bp, about 5000-15000 bp, about 5000-10000 bp, about 7500-25000 bp, about 7500-20000, about 7500-15000 bp, about 7500-10000 bp, about 10000-25000 bp, about 10000-20000 bp, about 10000-15000 bp, about 15000-25000 bp, about 15000-20000 bp, about 20000-25000 bp, about 7000-9000 bp, or about 8000 bp in length).
   E223. The plurality of fragments of any one of embodiments E181-E222, wherein one or more of the fragments (e.g., subgenomic fragments) of the plurality are present in a first carrier vector.
   E224. The plurality of fragments of embodiment E223, wherein each of the one or more fragments (e.g., subgenomic fragments) are present in separate first carrier vectors.
   E225. The plurality of fragments of any one of embodiments E181-E222, wherein one or more of the fragments (e.g., subfragments) of the plurality are present in a second carrier vector.
   E226. The plurality of fragments of embodiment E225, wherein each of one or more fragments (e.g., subfragments) are present in separate second carrier vectors.
   E227. The plurality of fragments of embodiment E223 or E224, wherein each of the one or more fragments (e.g., subgenomic fragments) is generated from a first carrier vector or separate first carrier vectors using the same restriction enzyme, e.g., the same type II restriction enzyme (e.g., type IIS restriction enzyme).
   E228. The plurality of fragments of embodiment E225 or E226, wherein each of the one or more fragments (e.g., subfragments) is generated from a second carrier vector or separate second carrier vectors using the same restriction enzyme, e.g., the same type II restriction enzyme (e.g., the same type IIS restriction enzyme), wherein the same restriction enzyme is different from the restriction enzyme used to generate fragments from the first carrier vector or separate first carrier vectors.
   E229. The plurality of fragments of any one of embodiments E220-E228, wherein one, two, three or all of the restriction enzyme, the first restriction enzyme and/or the second restriction enzyme comprises one, two, three, four, or all of the following properties:
(i) is capable of producing a 5′ overhang upon cleavage by the restriction enzyme;
(ii) cleavage by the restriction enzyme results in at least one or at least two (e.g., one or two) base pairs outside the 5′ overhang in its recognition site;
(iii) is capable of producing a 3′ overhang upon cleavage by the restriction enzyme; (iv) is heat inactivatable; and/or
(v) recognizes a stretch of at least 4-8 base pairs, e.g., at least 4 base pairs, at least 5 base pairs, at least 6 base pairs, at least 7 base pairs, or at least 8 base pairs (e.g., 8 base pairs).
   E230. The plurality of fragments of any one of embodiments E220-E229, wherein the restriction enzyme, the first restriction enzyme and/or the second restriction enzyme is a type II restriction enzyme.
   E231. The plurality of fragments of any one of embodiments E220-E230, wherein the restriction enzyme, the first restriction enzyme and/or the second restriction enzyme is a restriction enzyme that is able to be used in Gibson Assembly™ cloning and ligation method (e.g., a method as described in Gibson et al. “Enzymatic assembly of DNA molecules up to several hundred kilobases,” Nat. Methods, 2009, 6(5):343-5; the contents of which are hereby incorporated by reference in their entirety), e.g., a Gibson Assembly compatible enzyme.
   E232. The plurality of fragments of any one of embodiments E220-E231, wherein the restriction enzyme, the first restriction enzyme and/or the second restriction enzyme is selected from AcuI, AlwI, AaR1, BaeI, BbsI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, EarI, EciI, Esp3I, FauI, FokI, HgaI, HphI, HpyAV, MbolI, MlyI, MnlI, Nme-AIII, PaqCI, PleI, SapI, SfaNI, AbsI, Acc65I, AgeI, AgsI, ArsI, AscI, AsiGI, AsiSI, Asp718I, AspA2I, AsuNHI, AvrII, Alw44I, AxyI, BamHI, BelI, BcnI, BglII, Bpu1102I, BlnI, BmtI, BsaI, Bse21I, BshTI, BsmBI, Bso31I, BspOI, BspTNI, BstENI, Bsp119I, Bsp120I, Bsu15I, Bsu36I, CciNI, Cfr13I, Csp6I, Cfr9I, CspAI, Ec1136II, Eco31I, Eco53kI, Eco81I, Eco91I, Eco52I, EcoICRI, EcoNI, EcoRI, Esp3I, FseI, Hin6I, HindIII, HpaII, I-CeuI, I-PpoI, I-SceI, KpnI, Kpn2I, LmnI, LpnPI, MauBI, MspI, MluI, NcoI, NdeI, NheI, NotI, PalAI, PI-PspI, PI-SceI, PinAI, Psp124BI, RgaI, RigI, SacI, SalI, SbfI, SdaI, SfaAI, SgfI, SgsI, SmaI, SrfI, Sse83871, SstI, TaqI, TspMI, XagI, XbaI, XhoI, XmaI, XmaCI, XmaJI, MunI, HinfI, BspTI, Pfl23II, VspI, Bsp1407I, Psp1406I, Tru1I, Bpu10I, BcuI, PagI, BshTI, XmaJI, PfoI, SgsI, PteI, KflI, or an isoschizomer of any of the aforesaid restriction enzymes.
   E233. The plurality of fragments of any one of embodiments E220-E232, wherein the restriction enzyme, the first restriction enzyme and/or the second restriction enzyme is a type IIS restriction enzyme.
   E234. The plurality of fragments of embodiment E233, wherein the type IIS restriction enzyme is selected from AcuI, AlwI, AaR1, BaeI, BbsI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, EarI, EciI, Esp3I, FauI, FokI, HgaI, HphI, HpyAV, MbolI, MlyI, MnlI, Nme-AIII, PaqCI, PleI, SapI, or SfaNI, or an isoschizomer of any of the aforesaid restriction enzymes.
   E235. The plurality of fragments of embodiment E233 or E234, wherein the type IIS restriction enzyme is a BsaI restriction enzyme, a BsmBI restriction enzyme, a PaqCI restriction enzyme, or a combination thereof.
   E236. The plurality of fragments of any one of embodiments E220-E235, wherein:
(i) the first restriction enzyme is BsmBI and the second restriction enzyme is BsaI; or
(ii) the first restriction enzyme is BsaI and the second restriction enzyme is BsmBI.
   E237. The plurality of fragments of any one of embodiments E220-E236, wherein the restriction enzyme, the first restriction enzyme and/or the second restriction enzyme is selected from AbsI, Acc65I, AgeI, AgsI, ArsI, AscI, AsiGI, AsiSI, Asp718I, AspA2I, AsuNHI, AvrII, Alw44I, AxyI, BamHI, BelI, BcnI, BglII, Bpu1102I, BlnI, BmtI, BsaI, Bse21I, BshTI, BsmBI, Bso31I, BspOI, BspTNI, BstENI, Bsp119I, Bsp120I, Bsu15I, Bsu36I, CciNI, Cfr13I, Csp6I, Cfr9I, CspAI, Ec1136II, Eco31I, Eco53kI, Eco81I, Eco91I, Eco52I, EcoICRI, EcoNI, EcoRI, Esp3I, FseI, Hin6I, HindIII, HpaII, I-CeuI, I-PpoI, I-SceI, KpnI, Kpn2I, LmnI, LpnPI, MauBI, MspI, MluI, NcoI, NdeI, NheI, NotI, PalAI, PI-PspI, PI-SceI, PinAI, Psp124BI, RgaI, RigI, SacI, SalI, SbfI, SdaI, SfaAI, SgfI, SgsI, SmaI, SrfI, Sse83871, SstI, TaqI, TspMI, XagI, XbaI, XhoI, XmaI, XmaCI, XmaJI, MunI, HinfI, BspTI, Pfl23II, VspI, Bsp1407I, Psp1406I, Tru1I, Bpu101, BcuI, PagI, BshTI, XmaJI, PfoI, SgsI, PteI, KflI, or an isoschizomer of any of the aforesaid restriction enzymes.
   E238. The plurality of fragments of any one of embodiments E220-E237, wherein the restriction enzyme, the first restriction enzyme and/or the second restriction enzyme is AgeI, AvrII, BamHI, NheI, AscI, NotI, or an isoschizomer of any of the aforesaid restriction enzymes.
   E239. The plurality of fragments of any one of embodiments E220-E238, wherein the restriction enzyme, the first restriction enzyme and/or the second restriction enzyme is AgeI, AvrII, BamHI, NheI, or an isoschizomer of any of the aforesaid restriction enzymes.
   E240. The plurality of fragments of any one of embodiments E220-E239, wherein the restriction enzyme, the first restriction enzyme and/or the second restriction enzyme is AscI, NotI, or an isoschizomer of any of the aforesaid restriction enzymes.
   E241. The plurality of fragments of any one of embodiments E181-E240, wherein the variant baculovirus nucleotide sequence of one or more of the fragments of the plurality comprises a modification, e.g., an insertion, deletion, or substitution.
   E242. The plurality of fragments of embodiment E241, wherein the modification, e.g., an insertion, deletion, substitution, or mutation (e.g., frame-shift mutation), is present in a non-essential gene (e.g., auxiliary and/or per os infectivity factor gene) or a regulatory region of a non-essential gene (e.g., promoter modification or insertion of heterologous DNA adjacent to a non-essential gene).
   E243. The plurality of fragments of embodiment E242, wherein the non-essential gene is selected from one, two, three, or more (e.g., 1, 2, 3, 4, 5, 6, 7, 8, 9, 10, 11, 12, 13, 14, 15, 16, 17, 18, 19, 20, or 21) of egt, p74 (PIF0), p26, SOD, ChiA, v-cath, p10, polyhedrin, ctx, odv-e56, PIF1, PIF2, PIF3, PIF4, PIF5, Tn7, AcORF-91, AcORF-108, AcORF-52, v-ubi, or p94.
   E244. The plurality of fragments of embodiment E243, which comprises a modification, e.g., an insertion, deletion, substitution, or mutation (e.g., frame-shift mutation) in:
(i) v-cath and egt;
(ii) v-cath, egt, and SOD;
(iii) chiA, v-cath, egt, p26, p10, and p74;
(iv) chiA, v-cath, egt, p26, p10, p74, and SOD; or
(v) chiA, v-cath, egt, p26, p10, p74, SOD, AcORF-91, and AcORF-108.
   E245. The plurality of fragments of any one of embodiments E241-E244, wherein the modification comprises a deletion of a chiA gene, a v-cath gene, a p26 gene, a p10 gene, and/or a p74 gene, or a portion thereof.
   E246. The plurality of fragments of any one of embodiments E242-E245, wherein the modification comprises an insertion of a heterologous sequence in the non-essential gene or adjacent region.
   E247. The plurality of fragments of any one of embodiments E242-E246, wherein the modification comprises one or more mutations in the non-essential gene or adjacent region.
   E248. The plurality of fragments of any one of embodiments E242-E247, wherein the non-essential genes are present near (e.g., downstream or upstream) of a homologous repeat region 5 (hr5).
   E249. The plurality of fragments of any one of embodiments E241-E248, wherein the modification, e.g., an insertion, deletion, substitution, or mutation (e.g., frame-shift mutation), results in inactivation of a non-essential gene (e.g., auxiliary and/or per os infectivity factor gene) or a regulatory region of a non-essential gene (e.g., promoter modification or insertion of heterologous DNA adjacent to non-essential gene).
   E250. The plurality of fragments of embodiment E181-E249, wherein the variant baculovirus nucleotide sequence of one or more of the fragments of the plurality comprises a disruption, e.g., a mutation (e.g., frame-shift mutation), a deletion, an insertion, or inactivation, of a non-essential gene (e.g., auxiliary and/or per os infectivity factor gene) or a regulatory region of a non-essential gene (e.g., promoter modification or insertion of heterologous DNA adjacent to non-essential gene).
   E251. The plurality of fragments of any one of embodiments E241-E250, wherein the modification is introduced by chemical synthesis or PCR, e.g., PCR-based site-directed mutagenesis.
   E252. The plurality of fragments of any one of embodiments E181-E251, wherein one or more of the fragments of the plurality comprises a heterologous nucleotide sequence.
   E253. The plurality of fragments of embodiment E252, wherein the heterologous nucleotide sequence comprises a sequence of interest (e.g., a nucleotide sequence encoding a Rep-coding region, one or more Rep proteins, a VP-coding region, one or more VP proteins, and/or a payload).
   E254. The plurality of fragments of embodiment E253, wherein the sequence of interest is a nucleotide sequence encoding a polypeptide of interest or a nucleotide sequence of interest.
   E255. The plurality of fragments of embodiment E253 or E254, wherein the sequence of interest is operably linked to a first promoter (e.g., a baculovirus early promoter or a baculovirus early-late promoter) and/or a second promoter (e.g., a baculovirus late promoter or a baculovirus very late promoter).
   E256. The plurality of fragments of embodiment E253-E255, wherein the sequence of interest is operably linked to a first promoter (e.g., a baculovirus early promoter or a baculovirus early-late promoter) and a second promoter (e.g., a baculovirus late promoter or a baculovirus very late promoter).
   E257. The plurality of fragments of embodiment E255 or E256, wherein the first and/or second promoter is selected from a baculovirus promoter, a viral promoter, an insect viral promoter, a non-insect viral promoter, a vertebrate viral promoter, a chimeric promoter from one or more species including virus and non-virus elements, a synthetic promoter, or a variant thereof.
   E258. The plurality of fragments of any one of embodiments E255-E257, wherein the first and/or second promoter chosen from a polh promoter, a p10 promoter, a ctx promoter, a gp64 promoter, an IE promoter, an IE-1 promoter, a p6.9 promoter, a Dmhsp70 promoter, a Hsp70 promoter, a p5 promoter, a p19 promoter, a p35 promoter, a p40 promoter, or a variant, e.g., functional fragment, thereof.
   E259. The plurality of fragments of any one of embodiments E255-E258, wherein the first and/or second promoter is selected from an a baculovirus early promoter, baculovirus late promoter, baculovirus early-late promoter, or a baculovirus very late promoter.
   E260. The plurality of fragments of any one of embodiments E255-E259, wherein first and/or second promoter is a baculovirus early promoter, baculovirus late promoter, or baculovirus early-late promoter.
   E261. The plurality of fragments of any one of embodiments E255-E260, wherein first and/or second promoter is a baculovirus early-late promoter (e.g., a gp64 promoter).
   E262. The plurality of fragments of any one of embodiments E255-E261, wherein the first or second promoter is a baculovirus very late promoter (e.g., a polh promoter).
   E263. The plurality of fragments of any one of embodiments E255-E262, wherein:
(a) the first promoter is an baculovirus early-late promoter and the second promoter is a baculovirus very late promoter,
(b) the first promoter is a baculovirus very late promoter and the second promoter is a baculovirus early-late promoter,
(c) the first promoter is a baculovirus early promoter and the second promoter is a baculovirus early-late promoter,
(d) the first promoter is a baculovirus early-late promoter and the second promoter is a baculovirus early promoter,
(e) the first promoter is a baculovirus early promoter and the second promoter is a baculovirus late promoter,
(f) the first promoter is a baculovirus late promoter and the second promoter is a baculovirus early promoter,
(g) the first promoter is a baculovirus early-late promoter and the second promoter is a baculovirus late promoter,
(h) the first promoter is a baculovirus late promoter and the second promoter is a baculovirus early-late promoter,
(i) the first promoter is a baculovirus late promoter and the second promoter is a baculovirus very-late promoter,
(j) the first promoter is a baculovirus very-late promoter and the second promoter is a baculovirus late promoter,
(k) the first promoter is a baculovirus early promoter and the second promoter is a baculovirus very late promoter,
(l) the first promoter is a baculovirus very late promoter and the second promoter is a baculovirus early promoter,
(m) the first promoter is a baculovirus early promoter and the second promoter is a baculovirus early promoter,
(n) the first promoter is a baculovirus early-late promoter and the second promoter is a baculovirus early-late promoter, or
(o) the first promoter is a baculovirus late promoter and the second promoter is a baculovirus late promoter.
   E264. The plurality of fragments of any one of embodiments E255-E263, wherein the first promoter is a baculovirus early-late promoter (e.g., gp64 promoter) and the second promoter is a baculovirus very late promoter (e.g., polh promoter).

E265. The plurality of fragments of any one of embodiments E255-E264, wherein the first promoter is a baculovirus early promoter and the second promoter is a baculovirus late promoter.

E266. The plurality of fragments of any one of embodiments E255-E265, wherein
(a) the baculovirus early promoter is selected from: a lef3 promoter, a dbp promoter, a p35 promoter, an orf82 promoter, an get promoter, an orf81 promoter, an orf122 promoter, a pk-2 promoter, an orf55 promoter, an etl promoter, a hcf-1 promoter, an etm promoter, a lef-2 promoter, a lef-6 promoter, an orf84 promoter, an orf118 promoter, or an orf111 promoter,
(b) the baculovirus early-late promoter is selected from: a lef2 promoter, a orf13 promoter, a orf23 promoter, a pkip promoter, a v-fgf promoter, a pp31 promoter, an odv-e66 promoter, an orf74 promoter, an orf79 promoter, an orf82 promoter, a p15 promoter, a cg30 promoter, a helicase promoter, an he65 promoter, an orf114 promoter, a pk-2 promoter, a gp64 promoter, a gp16 promoter, an alk-exo promoter, a p35 promoter, a me53 promoter, or an ie0 promoter,
(c) the baculovirus late promoter is selected from: a ptpase promoter, an Ac-bro promoter, a ctx promoter, an orf5 promoter, an orf19 promoter, an orf20 promoter, an sod promoter, a HisP promoter, an orf34 promoter, a v-ubi promoter, an orf38 promoter, an orf43 promoter, an orf44 promoter, an orf56 promoter, an orf59 promoter, an orf60 promoter, or an fp-25k promoter, and/or
(d) the baculovirus very late promoter is selected from a p10 promoter or a polh promoter.
   E267. The plurality of fragments of any one of embodiments E255-E266, wherein the first and/or second promoter comprises a TATA box motif and/or a CAGT motif.
   E268. The plurality of fragments of any one of embodiments E255-E267, wherein the first and/or second promoter comprises a TAAG motif (e.g., an ATAAG nucleotide sequence).
   E269. The plurality of fragments of any one of embodiments E255-E268, wherein the first and/or second promoter comprises both a TATA box motif and a TAAG motif.
   E270. The plurality of fragments of any one of embodiments E255-E269, wherein the first or second promoter comprises a binding site for VLF-1.
   E271. The plurality of fragments of any one of embodiments E255-E270, wherein the first or second promoter is a gp64 promoter (e.g., an OpMNPV gp64 promoter).
   E272. The plurality of fragments of any one of embodiments E255-E271, wherein the first or second promoter is a polh promoter (e.g., an OpMNPV polh promoter or an AcMNPV polh promoter).
   E273. The plurality of fragments of any one of embodiments E255-E272, wherein the first promoter is a gp64 promoter and the second promoter is a polh promoter, or wherein the first promoter is a polh promoter and the second promoter is a gp64 promoter.
   E274. The plurality of fragments of any one of embodiments E255-E273, wherein the Rep-coding region is operably linked to a first promoter which is a baculovirus early-late promoter and a second promoter which is baculovirus very late promoter, e.g., a gp64 promoter and a polh promoter, optionally, wherein the Rep-coding region is present downstream of a homologous repeat region hr5.
   E275. The plurality of fragments of any one of embodiments E255-E274, wherein the first promoter is a gp64 promoter and the second promoter is a polh promoter.
   E276. The plurality of fragments of embodiment E255-E263 or E266-E272, wherein the first promoter and the second promoter are the same.
   E277. The plurality of fragments of embodiment E255-E275, wherein the first promoter and the second promoter are different.
   E278. The plurality of fragments of embodiment E277, the first promoter and the second promoter are each a polh promoter.
   E279. The plurality of fragments of embodiment E258-E277, wherein the gp64 promoter comprises the nucleotide sequence of SEQ ID NO: 217; a nucleotide sequence at least 70%, 75%, 80%, 85%, 90%, 95%, 96%, 97%, 98%, or 99% identical thereto; a nucleotide sequence comprising at least one, two, three, four, five, six, or seven, but no more than ten different nucleotides relative to SEQ ID NO: 217; or a nucleotide sequence comprising at least one, two, three, four, five, six, or seven, but no more than ten modifications (e.g., substitutions) relative to SEQ ID NO: 217.
   E280. The plurality of fragments of embodiment E258-E278, wherein the polh promoter comprises the nucleotide sequence of SEQ ID NO: 167 or 220; a nucleotide sequence at least 70%, 75%, 80%, 85%, 90%, 95%, 96%, 97%, 98%, or 99% identical thereto; a nucleotide sequence comprising at least one, two, three, four, five, six, or seven, but no more than ten different nucleotides relative to SEQ ID NO: 167 or 220; or a nucleotide sequence comprising at least one, two, three, four, five, six, or seven, but no more than ten modifications (e.g., substitutions) relative to SEQ ID NO: 167 or 220.
   E281. The plurality of fragments of any one of embodiments E258-E277, wherein the first promoter and the second promoter comprises the nucleotide sequence of SEQ ID NO: 221; a nucleotide sequence at least 70%, 75%, 80%, 85%, 90%, 95%, 96%, 97%, 98%, or 99% identical thereto; a nucleotide sequence comprising at least one, two, three, four, five, six, or seven, but no more than ten different nucleotides relative to SEQ ID NO: 221; or a nucleotide sequence comprising at least one, two, three, four, five, six, or seven, but no more than ten modifications (e.g., substitutions) relative to SEQ ID NO: 221.
   E282. The plurality of fragments of any one of embodiments E253-E282, wherein the sequence of interest encodes a therapeutic protein or functional variant thereof, an antibody or antibody fragment; an enzyme; a component of a gene editing system; an RNAi agent (e.g., a dsRNA, siRNA, shRNA, pre-miRNA, pri-miRNA, miRNA, stRNA, lncRNA, piRNA, or snoRNA); or a combination thereof.
   E283. The plurality of fragments of any one of embodiments E181-E282, wherein one or more of the fragments of the plurality (e.g., subgenomic fragments or subfragments) encodes one or more AAV proteins.
   E284. The plurality of fragments of any one of embodiments E181-E283, wherein one or more of the fragments of the plurality (e.g., subgenomic fragments or subfragments) encodes an AAV Rep protein, e.g., Rep40, Rep52, Rep68, Rep78, or a combination thereof.
   E285. The plurality of fragments of any one of embodiments E181-E284, wherein one or more of the fragments of the plurality (e.g., subgenomic fragments or subfragments) encodes a Rep78 protein and/or a Rep52 protein.
   E286. The plurality of fragments of any one of embodiments E181-E285, wherein one or more of the fragments of the plurality (e.g., subgenomic fragments or subfragments) encodes an AAV capsid protein, e.g., a VP1 protein, a VP2 protein, a VP3, protein or a combination thereof.
   E287. The plurality of fragments of any one of embodiments E181-E286, wherein one or more of the fragments of the plurality (subgenomic fragments or subfragments) encodes an AAV1 capsid protein, an AAV2 capsid protein, an AAV3 capsid protein, an AAV4 capsid protein, an AAV5 capsid protein, an AAV6 capsid protein, an AAV8 capsid protein, an AAV9 capsid protein, an AAVrh10 capsid protein, or a variant thereof.
   E288. The plurality of fragments of any one of embodiments E181-E287, wherein one or more of the fragments of the plurality (e.g., subgenomic fragments or subfragments) encodes an AAV5 capsid protein or variant thereof, or an AAV9 capsid protein or variant thereof.
   E289. The plurality of fragments of any one of embodiments E181-E288, wherein one or more of the fragments of the plurality (e.g., subgenomic fragments or subfragments) encodes a payload.
   E290. The plurality of fragments of embodiment E289, wherein the encoded payload is selected from a therapeutic protein or functional variant thereof; an antibody or antibody fragment; an enzyme; a component of a gene editing system; an RNAi agent (e.g., a dsRNA, siRNA, shRNA, pre-miRNA, pri-miRNA, miRNA, stRNA, lncRNA, piRNA, or snoRNA); or a combination thereof.
   E291. The plurality of fragments of any one of embodiments E181-E290, wherein one or more of the fragments of the plurality (e.g., subgenomic region or subfragments) are chemically synthesized or are non-templated fragments.
   E292. A variant baculovirus genome which comprises at least 5 fewer functional restriction enzyme sites (e.g., functional naturally occurring restriction enzyme sites), e.g., type II restriction enzyme sites (e.g., type IIS restriction enzyme sites), relative to a reference baculovirus genome, e.g., a wild-type baculovirus genome, optionally wherein the baculovirus expression construct is replication-competent.
   E293. The variant baculovirus genome of embodiment E292, which comprises at least 10, at least 15, at least 20, at least 25, at least 30, at least 35, at least 40, at least 45, at least 50, at least 55, or at least 60 fewer, e.g., 15-60, 15-50, 15-40, 15-30, 15-20, 20-60, 20-50, 20-40, 20-30, 30-60, 30-50, 30-40, 40-60, 40-50, or 50-60 fewer functional restriction enzyme sites (e.g., functional naturally occurring restriction enzyme sites), e.g., type II restriction enzyme sites (e.g., type IIS restriction enzyme sites), relative to a reference baculovirus genome, e.g., a nucleotide sequence in a wild-type baculovirus genome.
   E294. The variant baculovirus genome of embodiment E292 or E293, wherein the variant baculovirus nucleotide sequence comprises:
(i) at least 10, at least 15, at least 20, at least 25, at least 30, at least 35, at least 40, at least 45, at least 50, at least 55, or at least 60 fewer, e.g., 15-60, 15-50, 15-40, 15-30, 15-20, 20-60, 20-50, 20-40, 20-30, 30-60, 30-50, 30-40, 40-60, 40-50, or 50-60 fewer, functional recognition sites (e.g., functional naturally occurring restriction enzyme sites) of a type II restriction enzyme, e.g., a selected type II restriction enzyme relative to a nucleotide sequence in a reference baculovirus genome, e.g., a nucleotide sequence in a wild-type baculovirus genome; or
(ii) at least 10, at least 15, at least 20, at least 25, at least 30, at least 35, at least 40, at least 45, at least 50, at least 55, or at least 60 fewer, e.g., 15-60, 15-50, 15-40, 15-30, 15-20, 20-60, 20-50, 20-40, 20-30, 30-60, 30-50, 30-40, 40-60, 40-50, or 50-60 fewer, functional recognition sites (e.g., functional naturally occurring restriction enzyme sites) of two or more (e.g., 2-5, 2, 3, 4, or 5) selected type II restriction enzymes relative to a nucleotide sequence in a reference baculovirus genome, e.g., a nucleotide sequence in a wild-type baculovirus genome.
   E295. The variant baculovirus genome of any one of embodiments E292-E294, wherein the variant baculovirus nucleotide sequence comprises at least 10, at least 15, at least 20, at least 25, at least 30, at least 35, at least 40, at least 45, at least 50, at least 55, or at least 60 fewer, e.g., 15-60, 15-50, 15-40, 15-30, 15-20, 20-60, 20-50, 20-40, 20-30, 30-60, 30-50, 30-40, 40-60, 40-50, or 50-60 fewer, functional type IIS restriction enzyme sites (e.g., functional naturally occurring type IIS restriction enzyme sites) relative to a nucleotide sequence in a reference baculovirus genome, e.g., a nucleotide sequence in a wild-type baculovirus genome.
   E296. The variant baculovirus genome of any one of embodiments E292-E295, wherein the variant baculovirus nucleotide sequence comprises:
(i) at least 10, at least 15, at least 20, at least 25, at least 30, at least 35, at least 40, at least 45, at least 50, at least 55, or at least 60 fewer, e.g., 15-60, 15-50, 15-40, 15-30, 15-20, 20-60, 20-50, 20-40, 20-30, 30-60, 30-50, 30-40, 40-60, 40-50, or 50-60 fewer, functional recognition sites (e.g., functional naturally occurring restriction enzyme sites) of a selected type IIS restriction enzyme relative to a nucleotide sequence in a reference baculovirus genome, e.g., a nucleotide sequence in a wild-type baculovirus genome; or
(ii) at least 10, at least 15, at least 20, at least 25, at least 30, at least 35, at least 40, at least 45, at least 50, at least 55, or at least 60 fewer, e.g., 15-60, 15-50, 15-40, 15-30, 15-20, 20-60, 20-50, 20-40, 20-30, 30-60, 30-50, 30-40, 40-60, 40-50, or 50-60 fewer, functional recognition sites (e.g., functional naturally occurring restriction enzyme sites) of two or more (e.g., 2-5, 2, 3, 4, or 5) selected type IIS restriction enzymes relative to a nucleotide sequence in a reference baculovirus genome, e.g., a nucleotide sequence in a wild-type baculovirus genome.
   E297. The variant baculovirus genome of any one of embodiments E292-E296, wherein the variant baculovirus nucleotide sequence comprises:
(i) no functional recognition sites (e.g., functional naturally occurring recognition sites) of two or more (e.g., 2, 3, 4, or 5) selected type II restriction enzymes, e.g., selected type IIS restriction enzymes;
(ii) no functional recognition sites (e.g., functional naturally occurring recognition sites) of a selected type II restriction enzyme, e.g., selected type IIS restriction enzyme; or
(iii) no functional recognition sites (e.g., functional naturally occurring recognition sites) for type II restriction enzymes, e.g., selected type IIS restriction enzymes.
   E298. The variant baculovirus genome of any one of embodiments E292-E297, wherein the variant baculovirus nucleotide sequence is devoid of:
(i) recognition sites (e.g., functional naturally occurring recognition sites) of a selected type II restriction enzyme;
(ii) recognition sites (e.g., functional naturally occurring recognition sites) for 1-5 (e.g., 1-4, 1-3, 1-2, 1, 2, 3, 4, or 5) selected type II restriction enzymes; or
(iii) recognition sites (e.g., functional naturally occurring recognition sites) for type II restriction enzymes.
   E299. The variant baculovirus genome of embodiment E292-E298, wherein the restriction enzyme sites are recognition sites for a selected type IIS restriction enzyme.
   E300. The variant baculovirus genome of any one of embodiments E292-E299, wherein the type IIS restriction sites are recognition sites for two or more (e.g., 2, 3, 4, or 5) selected type IIS restriction enzymes.
   E301. The variant baculovirus genome of any one of embodiments E292-E300, wherein the variant baculovirus nucleotide sequence is devoid of recognition sites of a selected type IIS restriction enzyme.
   E302. The variant baculovirus genome of any one of embodiments E292-E301, wherein the variant baculovirus nucleotide sequence is devoid of recognition sites of two or more (e.g., 2-5, 2, 3, 4, or 5) selected type IIS restriction enzymes.
   E303. The variant baculovirus genome of any one of embodiments E292-E302, wherein the variant baculovirus nucleotide sequence is devoid of type IIS restriction enzyme sites.
   E304. The variant baculovirus genome of any one of embodiments E292-E303, wherein the restriction enzyme site is recognized by a restriction enzyme having one, two, three, four, or all of the following properties:
(i) is capable of producing a 5′ overhang upon cleavage by the restriction enzyme;
(ii) cleavage by the restriction enzyme results in at least one or at least two (e.g., one or two) base pairs outside the 5′ overhang in its recognition site;
(iii) is capable of producing a 3′ overhang upon cleavage by the restriction enzyme;
(iv) is heat inactivatable;
(v) recognizes a stretch of at least 4-8 base pairs, e.g., at least 4 base pairs, at least 5 base pairs, at least 6 base pairs, at least 7 base pairs, or at least 8 base pairs (e.g., 8 base pairs).
   E305. The variant baculovirus genome of any one of E292-E304, wherein the restriction enzyme site is a type II restriction enzyme site.
   E306. The variant baculovirus genome of any one of embodiments E292-E305, wherein the restriction enzyme site is recognized by a restriction enzyme that is able to be used in a Gibson Assembly™ cloning and ligation method (e.g., a method as described in Gibson et al. “Enzymatic assembly of DNA molecules up to several hundred kilobases,” Nat. Methods, 2009, 6(5):343-5; the contents of which are hereby incorporated by reference in their entirety), e.g., a Gibson Assembly compatible enzyme.
   E307. The variant baculovirus genome of any one of embodiments E292-E306, wherein the restriction enzyme site is recognized by a restriction enzyme selected from AcuI, AlwI, AaR1, BaeI, BbsI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, EarI, EciI, Esp3I, FauI, FokI, HgaI, HphI, HpyAV, MbolI, MlyI, MnlI, Nme-AIII, PaqCI, PleI, SapI, SfaNI, AbsI, Acc65I, AgeI, AgsI, ArsI, AscI, AsiGI, AsiSI, Asp718I, AspA2I, AsuNHI, AvrII, Alw44I, AxyI, BamHI, BelI, BcnI, BglII, Bpu1102I, BlnI, BmtI, BsaI, Bse21I, BshTI, BsmBI, Bso31I, BspOI, BspTNI, BstENI, Bsp1191, Bsp120I, Bsu15I, Bsu36I, CciNI, Cfr13I, Csp6I, Cfr9I, CspAI, Ec1136II, Eco31I, Eco53kI, Eco81I, Eco91I, Eco52I, EcoICRI, EcoNI, EcoRI, Esp3I, FseI, Hin6I, HindIII, HpaII, I-CeuI, I-PpoI, I-SceI, KpnI, Kpn2I, LmnI, LpnPI, MauBI, MspI, MluI, NcoI, NdeI, NheI, NotI, PalAI, PI-PspI, PI-SceI, PinAI, Psp124BI, RgaI, RigI, SacI, SalI, SbfI, SdaI, SfaAI, SgfI, SgsI, SmaI, SrfI, Sse8387I, SstI, TaqI, TspMI, XagI, XbaI, XhoI, XmaI, XmaCI, XmaJI, MunI, HinfI, BspTI, Pfl23II, VspI, Bsp1407I, Psp1406I, Tru1I, Bpu10I, BcuI, PagI, BshTI, XmaJI, PfoI, SgsI, PteI, KflI, or an isoschizomer of any of the aforesaid restriction enzymes.
   E308. The variant baculovirus genome of any one of embodiments E292-E307, wherein the restriction enzyme site is a type IIS restriction enzyme site.
   E309. The variant baculovirus genome of any one of embodiments E292-E308, wherein the type IIS restriction enzyme site is recognized by a restriction enzyme selected from AcuI, AlwI, AaR1, BaeI, BbsI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, EarI, EciI, Esp3I, FauI, FokI, HgaI, HphI, HpyAV, MbolI, MlyI, MnlI, Nme-AIII, PaqCI, PleI, SapI, SfaNI, or an isoschizomer of any of the aforesaid restriction enzymes.
   E310. The variant baculovirus genome of any one of embodiments E292-E309, wherein the type IIS restriction enzyme site is a BsmBI restriction enzyme site, a BsaI restriction enzyme site, a PaqCI restriction enzyme site, or a combination thereof.
   E311. The variant baculovirus genome of any one of embodiments E292-E310, wherein the type IIS restriction enzyme site is a BsmBI restriction enzyme site.
   E312. The variant baculovirus genome of any one of embodiments E292-E311, wherein the restriction enzyme site is recognized by a restriction enzyme selected from AbsI, Acc65I, AgeI, AgsI, ArsI, AscI, AsiGI, AsiSI, Asp718I, AspA2I, AsuNHI, AvrII, Alw44I, AxyI, BamHI, BelI, BcnI, BglII, Bpu1102I, BlnI, BmtI, BsaI, Bse21I, BshTI, BsmBI, Bso31I, BspOI, BspTNI, BstENI, Bsp1191, Bsp120I, Bsu15I, Bsu36I, CciNI, Cfr13I, Csp6I, Cfr9I, CspAI, Ec1136II, Eco31I, Eco53kI, Eco81I, Eco91I, Eco52I, EcoICRI, EcoNI, EcoRI, Esp3I, FseI, Hin6I, HindIII, HpaII, I-CeuI, I-PpoI, I-SceI, KpnI, Kpn2I, LmnI, LpnPI, MauBI, MspI, MluI, NcoI, NdeI, NheI, NotI, PalAI, PI-PspI, PI-SceI, PinAI, Psp124BI, RgaI, RigI, SacI, SalI, SbfI, SdaI, SfaAI, SgfI, SgsI, SmaI, SrfI, Sse8387I, SstI, TaqI, TspMI, XagI, XbaI, XhoI, XmaI, XmaCI, XmaJI, MunI, HinfI, BspTI, Pfl23II, VspI, Bsp14071, Psp14061, Tru1I, Bpu10I, BcuI, PagI, BshTI, XmaJI, PfoI, SgsI, PteI, KflI, or an isoschizomer of any of the aforesaid restriction enzymes.
   E313. The variant baculovirus genome of any one of embodiments E202-E312, wherein the restriction enzyme site is recognized by AgeI, AvrII, BamHI, NheI, AscI, NotI, or an isoschizomer of any of the aforesaid restriction enzymes.
   E314. The variant baculovirus genome of any one of embodiments E292-E313, wherein the restriction enzyme site is recognized by a restriction enzyme selected from AgeI, AvrII, BamHI, NheI, or an isoschizomer of any of the aforesaid restriction enzymes.
   E315. The variant baculovirus genome of any one of embodiments E292-E313, wherein the restriction enzyme site is recognized by a restriction enzyme selected from AscI, NotI, or an isoschizomer of any of the aforesaid restriction enzymes.
   E316. The variant baculovirus genome of any one of embodiments E292-E315, wherein the at least one, two, three, four or more of the at least 5 fewer functional type IIS restriction enzyme sites are different type IIS restriction enzyme sites, e.g., a first type IIS restriction enzyme site and a second type IIS restriction enzyme site, optionally wherein:
(i) the first type IIS restriction enzyme site is recognized by a restriction enzyme selected from AcuI, AlwI, AaR1, BaeI, BbsI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, EarI, EciI, Esp3I, FauI, FokI, HgaI, HphI, HpyAV, MbolI, MlyI, MnlI, Nme-AIII, PaqCI, PleI, SapI, or SfaNI, or an isoschizomer of any of the preceding restriction enzymes; and
(ii) the second type IIS restriction enzyme site is recognized by a restriction enzyme selected from AcuI, AlwI, AaR1, BaeI, BbsI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, EarI, EciI, Esp3I, FauI, FokI, HgaI, HphI, HpyAV, MbolI, MlyI, MnlI, Nme-AIII, PaqCI, PleI, SapI, or SfaNI, or an isoschizomer of any of the preceding restriction enzymes.
   E317. The variant baculovirus genome of any one of embodiments E292-E316, wherein the at least 5 fewer functional type IIS restriction enzyme sites are the same type IIS restriction enzyme site, e.g., a first type IIS restriction enzyme site.
   E318. The variant baculovirus genome of any one of embodiments E292-E317, wherein at least one, two, three, four or more of the at least 5 fewer functional type IIS restriction enzyme sites are different type IIS restriction enzyme sites, e.g., a first type IIS restriction enzyme site and a second type IIS restriction enzyme site.
   E319. The variant baculovirus genome of any one of embodiments E292-E318, wherein the variant baculovirus genome comprises at least 3, 4, or 5-fold fewer functional first type II restriction enzyme sites (e.g., type IIS restriction enzyme sites, e.g., BsaI type II restriction enzyme sites), relative to a second type II restriction enzyme sites (e.g., type IIS restriction enzyme sites, e.g., BsmBI type II restriction enzyme sites).
   E320. The variant baculovirus genome of any one of embodiments E292-E319, wherein the variant baculovirus genome comprises at least 3, 4, or 5-fold more functional second type II restriction enzyme sites (e.g., type IIS restriction enzyme sites, e.g., BsmBI type II restriction enzyme sites), relative to a first type II restriction enzyme site (e.g., type IIS restriction enzyme site, e.g., BsaI type II restriction enzyme sites).
   E321. The variant baculovirus genome of any one of embodiments E292-E320, wherein the variant baculovirus genome comprises:
(i) at least 10-15 (e.g., 10-12, 10-13, 10-14, 11-15, 11-15, 11-13, 12-15, 12-14, 10, 11, 12, 13, 14, or 15) fewer functional BsaI type II restriction enzyme sites; and/or
(ii) at least 40-50 (e.g., 40-45, 41-50, 41-45, 42-50, 42-45, 43-50, 43-45, 44-50, 45-50, 46-50, 47-50, 48-50, 40, 41, 42, 43, 44, 45, 46, 47, 48, 49, or 50) fewer functional BsmBI type II restriction enzyme sites.
   E322. The variant baculovirus genome of any one of embodiments E292-E321, wherein the variant baculovirus genome comprises 11 fewer functional BsaI type II restriction enzyme sites and/or 45 fewer functional BsmBI type II restriction enzyme sites.
   E323. The variant baculovirus genome of any one of embodiments E292-E322, wherein the restriction site, e.g., type II restriction enzyme site (e.g., type IIS restriction enzyme site) comprises a modification, e.g., an insertion, deletion, or substitution, that renders the site inactive or incapable of being recognized by the corresponding restriction enzyme, e.g., type II restriction enzyme (e.g., type IIS restriction enzyme).
   E324. The variant baculovirus genome of any one of embodiments E292-E323, wherein the restriction site, e.g., type II restriction enzyme site (e.g., type IIS restriction enzyme site) comprises one, two, three, four, five or six modifications e.g., insertions, deletions, or substitutions, relative to the corresponding functional restriction site, e.g., type II restriction enzyme site (e.g., type IIS restriction enzyme site).
   E325. The variant baculovirus genome of any one of embodiments E292-E324, wherein the restriction site, e.g., type II restriction enzyme site (e.g., type IIS restriction enzyme site) comprises a modification, e.g., substitution, wherein the modification comprises a nucleotide from a different naturally occurring baculovirus genome sequence (e.g., a different species of a baculovirus of the same genus).
   E326. The variant baculovirus genome of any one of embodiments E292-E325, wherein the restriction site, e.g., type II restriction enzyme site (e.g., type IIS restriction enzyme site) comprises one, two, three, four, five or six different nucleotides relative to the corresponding functional restriction site, e.g., type II restriction enzyme site (e.g., type IIS restriction enzyme site).
   E327. The variant baculovirus genome of any one of embodiments E292-E326, wherein the restriction site, e.g., type II restriction enzyme site (e.g., type IIS restriction enzyme site) comprises a different nucleotide relative to the corresponding functional restriction site, e.g., type II restriction enzyme site (e.g., type IIS restriction enzyme site), wherein the different nucleotide is from a different naturally occurring baculovirus genome sequence.
   E328. The variant baculovirus genome of any one of embodiments E292-E327, wherein the variant baculovirus genome comprises a nucleotide sequence or a portion thereof from a baculovirus genome selected from Autographa californica multiple nucleopolyhedrovirus (AcMNPV) (e.g., an AcMNPV strain E2, C6, or HR3), Bombyx mori nucleopolyhedrovirus (BmNPV), Anticarsia gemmatalis nucleopolyhedrovirus (AgMNPV), Orgyia pseudotsugata nucleopolyhedrovirus (OpMNPV), or Thysanoplusia orichalcea nucleopolyhedrovirus (ThorMNPV).
   E329. The variant baculovirus genome of any one of embodiments E292-E38, wherein the variant baculovirus genome comprises a nucleotide sequence or a portion thereof from the AcMNPV (e.g., AcMNPV E2) baculovirus genome.
   E330. The variant baculovirus genome of any one of embodiments E292-E329, wherein the different naturally occurring baculovirus genome sequence comprises a nucleotide sequence or a portion thereof from a BmNPV baculovirus genome or a ThorMNPV baculovirus genome.
   E331. The variant baculovirus genome of any one of embodiments E292-E330, wherein the type IIS restriction enzyme site is absent or deleted.
   E332. The variant baculovirus genome of any one of embodiments E292-E331, wherein the reference baculovirus genome is a wild-type baculovirus genome or a modified baculovirus genome, e.g., a baculovirus genome having a deletion in at least one non-essential gene (e.g., auxiliary and/or per os infectivity factor gene), e.g., having a deletion in a polyhedrin (polH) locus.
   E333. The variant baculovirus genome of any one of embodiments E292-E332, wherein the reference baculovirus genome is a bMON14272 baculovirus genome.
   E334. The variant baculovirus genome of any one of embodiments E292-E333, wherein the modification is introduced by chemical synthesis or PCR, e.g., PCR-based site-directed mutagenesis.
   E335. The variant baculovirus genome of any one of embodiments E292-E334, wherein the variant baculovirus nucleotide genome comprises a disruption, e.g., an insertion, deletion, substitution, or mutation (e.g., frame-shift mutation), of a non-essential gene (e.g., auxiliary and/or per os infectivity factor gene) or a regulatory region of a non-essential gene (e.g., promoter modification or insertion of heterologous DNA adjacent to non-essential gene).
   E336. The variant baculovirus genome of any one of embodiments E292-E335, wherein the variant baculovirus genome comprises a disruption, e.g., a mutation (e.g., frameshift mutation), a deletion, an insertion, or inactivation, of at least 1, 2, 3, 4, 5, 10, 15, or 20 non-essential genes (e.g., auxiliary and/or per os infectivity factor genes).
   E337. The variant baculovirus genome of embodiment E335 or E336, wherein the non-essential gene is selected from one, two, three, or more (e.g., 1, 2, 3, 4, 5, 6, 7, 8, 9, 10, 11, 12, 13, 14, 15, 16, 17, 18, 19, 20, or 21) of egt, p74 (PIF0), p26, SOD, ChiA, v-cath, p10, polyhedrin, ctx, odv-e56, PIF1, PIF2, PIF3, PIF4, PIF5, Tn7, AcORF-91, AcORF-108, AcORF-52, v-ubi, or p94.
   E338. The variant baculovirus genome of embodiment E337, which comprises modification, e.g., an insertion, deletion, substitution, or mutation (e.g., frame-shift mutation) in:
(i) v-cath and egt;
(ii) v-cath, egt, and SOD;
(iii) chiA, v-cath, egt, p26, p10, and p74;
(iv) chiA, v-cath, egt, p26, p10, p74, and SOD; or
(v) chiA, v-cath, egt, p26, p10, p74, SOD, AcORF-91, and AcORF-108.
   E339. The variant baculovirus genome of any one of embodiments E332-E338, wherein the modification comprises a deletion of a chiA gene, a v-cath gene, a p26 gene, a p10 gene, and/or a p74 gene, or a portion thereof.
   E340. The variant baculovirus genome of any one of embodiments E332-E339, wherein the modification comprises an insertion of a heterologous sequence in the non-essential gene or adjacent region.
   E341. The variant baculovirus genome of any one of embodiments E332-E340, wherein the modification comprises one or more mutations in the non-essential gene or adjacent region.
   E342. The variant baculovirus genome of any one of embodiments E332-E341, wherein the non-essential genes are present near (e.g., downstream or upstream) of a homologous repeat region 5 (hr5).
   E343. The variant baculovirus genome of any one of embodiments E335-E342, wherein the disruption, e.g., a mutation (e.g., frameshift mutation), a deletion, an insertion, or inactivation, results in inactivation of a non-essential gene (e.g., auxiliary and/or per os infectivity factor gene) or a regulatory region of a non-essential gene (e.g., promoter modification or insertion of heterologous DNA adjacent to non-essential gene).
   E344. The variant baculovirus genome of any one of embodiments E335-E343, wherein the disruption is introduced by chemical synthesis or PCR, e.g., PCR-based site-directed mutagenesis.
   E345. The variant baculovirus genome of any one of embodiments E292-E344, wherein variant baculovirus genome comprises a heterologous nucleotide.
   E346. The variant baculovirus genome of embodiment E345, wherein the heterologous nucleotide sequence comprises a sequence of interest.
   E347. The variant baculovirus genome of embodiment E346, wherein the sequence of interest is a nucleotide sequence encoding a polypeptide of interest or a nucleotide sequence of interest.
   E348. The variant baculovirus genome of embodiment E345 or E346, wherein the sequence of interest encodes a therapeutic protein or functional variant thereof, an antibody or antibody fragment; an enzyme; a component of a gene editing system; an RNAi agent (e.g., a dsRNA, siRNA, shRNA, pre-miRNA, pri-miRNA, miRNA, stRNA, lncRNA, piRNA, or snoRNA); or a combination thereof.
   E349. The variant baculovirus genome of any one of embodiments E292-E348, wherein the variant baculovirus genome encodes one or more AAV proteins.
   E350. The variant baculovirus genome of any one of embodiments E292-E349, wherein the variant baculovirus genome encodes an AAV Rep protein, e.g., Rep40, Rep52, Rep68, Rep78, or a combination thereof.
   E351. The variant baculovirus genome of any one of embodiments E292-E350, wherein the variant baculovirus genome encodes a Rep78 protein and/or a Rep52 protein.
   E352. The variant baculovirus genome of any one of embodiments E292-E351, wherein the variant baculovirus genome encodes an AAV capsid protein, e.g., a VP1 protein, a VP2 protein, a VP3, protein or a combination thereof.
   E353. The variant baculovirus genome of any one of embodiments E292-E352, wherein the variant baculovirus genome encodes an AAV1 capsid protein, an AAV2 capsid protein, an AAV3 capsid protein, an AAV4 capsid protein, an AAV5 capsid protein, an AAV6 capsid protein, an AAV8 capsid protein, an AAV9 capsid protein, an AAVrh10 capsid protein, or a variant thereof.
   E354. The variant baculovirus genome of any one of embodiments E292-E353, wherein the variant baculovirus genome encodes an AAV5 capsid protein or variant thereof, or an AAV9 capsid protein or variant thereof.
   E355. The variant baculovirus genome of any one of embodiments E292-E354, wherein the variant baculovirus genome encodes a payload.
   E356. The variant baculovirus genome of embodiment E355, wherein the encoded payload is selected from a therapeutic protein or functional variant thereof; an antibody or antibody fragment; an enzyme; a component of a gene editing system; an RNAi agent (e.g., a dsRNA, siRNA, shRNA, pre-miRNA, pri-miRNA, miRNA, stRNA, lncRNA, piRNA, or snoRNA); or a combination thereof.
   E357. The variant baculovirus genome of any one of embodiments E292-E357, which further comprises an AAV expression construct.
   E358. The variant baculovirus genome of embodiment E357, wherein the AAV expression construct comprises:
(i) at least two Rep-coding regions, each comprising a nucleotide sequence encoding a Rep protein independently chosen from Rep52, Rep40, Rep68, or Rep78 protein, e.g., a Rep52 protein and a Rep78 protein; and/or
(ii) a VP-coding region comprising a nucleotide sequence encoding at least one, two, or three VP proteins, chosen from a VP1 protein, a VP2 protein, a VP3 protein, or a combination thereof,
optionally, wherein the at least two Rep-coding regions each comprise a different nucleotide sequence and/or are present in different locations.
   E359. The variant baculovirus genome of embodiment E357, wherein the AAV expression construct comprises:
(i) a Rep-coding region comprising a nucleotide sequence encoding a Rep protein chosen from Rep52, Rep40, Rep68, Rep78 protein, or a combination thereof, e.g., a Rep52 protein and/or a Rep78 protein; and
(ii) a VP-coding region comprising a nucleotide sequence encoding at least one, two, or three VP proteins chosen from a VP1 protein, a VP2 protein, a VP3 protein, or a combination thereof,
wherein the AAV expression construct comprises at least a portion of a baculovirus genome, e.g., a variant baculovirus genome, comprising a disruption of at least two non-essential genes (e.g., auxiliary and/or per os infectivity factor genes), wherein the at least two non-essential genes are independently chosen from egt, p74 (PIF0), p26, SOD, ChiA, v-cath, p10, polyhedrin, ctx, odv-e56, PIF1, PIF2, PIF3, PIF4, PIF5, Tn7, AcORF-91, AcORF-108, AcORF-52, v-ubi, or p94; and
wherein the Rep-coding region is operably linked to a first promoter, e.g., a baculovirus early promoter or a baculovirus early-late promoter (e.g., a gp64 promoter), and optionally a second promoter, e.g., a baculovirus later or a baculovirus very late promoter (e.g., a polh promoter), optionally, wherein:
(a) the first promoter results in transcription of the Rep-coding region prior to transcription of the VP-coding region;
(b) the Rep-coding region is present downstream of a homologous repeat region hr5; and/or
(c) the VP-coding region is present in the SOD locus.
   E360. The variant baculovirus genome of embodiment E358 or E359, wherein the VP-coding region comprises a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein.
   E361. The variant baculovirus genome of embodiment E358 or E360, wherein the at least two Rep-coding regions each comprise a different nucleotide sequence and is present in different locations in the variant baculovirus genome.
   E362. The variant baculovirus genome of any one of embodiments E358, E360, or E361, wherein the at least two Rep-coding regions comprise a first Rep-coding region and a second Rep-coding region.
   E363. The variant baculovirus genome of embodiment E362, wherein the first Rep-coding region comprises a first a first open reading frame (ORF) comprising a start codon and a nucleotide sequence encoding a Rep78 protein and the second Rep-coding region comprises a second ORF comprising a start codon and a nucleotide sequence encoding a Rep52 protein.
   E364. The variant baculovirus genome of embodiment E362 or E363, wherein the first Rep-coding region, the second Rep-coding region, or both comprises an ATG start codon (e.g., a canonical start codon).
   E365. The variant baculovirus genome of embodiment E362 or E363, wherein the first Rep-coding region, the second Rep-coding region, or both comprises an ACG start codon, a CTG start codon, a TTG start codon, or a GTG start codon (e.g., a non-canonical start codon).
   E366. The variant baculovirus genome of any one of embodiments E359 or E360, wherein the Rep-coding region comprises an ATG start codon.
   E367. The variant baculovirus genome of any one of embodiments E359, E360, or E366, wherein the Rep-coding region comprises a non-canonical start codon.
   E368. The variant baculovirus genome of any one of embodiments E359, E360, E366, or E367, wherein the Rep-coding region comprises an ACG start codon, a CTG start codon, a TTG start codon, or a GTG start codon.
   E369. The variant baculovirus genome of any one of embodiments E359, E360, or E366-E368, wherein the Rep-coding region comprises a CTG start codon.
   E370. The variant baculovirus genome of any one of embodiments E359, E360, or E366-E369, wherein the Rep-coding region comprises a ACG start codon.
   E371. The variant baculovirus genome of any one of embodiments E359, E360, or E366-E370, wherein the Rep-coding region comprises a TTG start codon.
   E372. The variant baculovirus genome of any one of embodiments E359, E360, or E366-E371, wherein the Rep-coding region comprises a GTG start codon.
   E373. The variant baculovirus genome of any one of embodiments E359, E360, or E366-E372, wherein
(i) the ORF encoding the Rep78 protein comprises an ATG start codon and the ORF encoding the Rep52 protein comprises an ATG start codon;
(ii) the ORF encoding the Rep78 protein comprises an CTG start codon and the ORF encoding the Rep52 protein comprises an ATG start codon;
(iii) the ORF encoding the Rep78 protein comprises an ATG start codon and the ORF encoding the Rep52 protein comprises an CTG start codon; or
(iv) the ORF encoding the Rep78 protein comprises an CTG start codon and the ORF encoding the Rep52 protein comprises an CTG start codon.
   E374. The variant baculovirus genome of any one of embodiments E362-E365, wherein the first Rep-coding region comprises a nucleotide sequence encoding a Rep78 protein.
   E375. The variant baculovirus genome of any one of embodiments E362-E365 or E374, wherein the first Rep-coding region comprises a nucleotide sequence encoding primarily a Rep78 protein, e.g., at least 50%, 60%, 70%, 80%, 90% or more Rep78 protein relative to a Rep52 protein.
   E376. The variant baculovirus genome of any one of embodiments E362-E365, E374, or E375, wherein the first Rep-coding region comprises a nucleotide sequence encoding a Rep78 protein only.
   E377. The variant baculovirus genome of any one of embodiments E362-E365 or E374, wherein the first Rep-coding region comprises a nucleotide sequence encoding a Rep78 protein but not a Rep52 protein.
   E378. The variant baculovirus genome of any one of embodiments E362-E365 or E374-E377, wherein the second Rep-coding comprises a nucleotide sequence encoding a Rep52 protein.
   E379. The variant baculovirus genome of any one of embodiments E362-E365 or E374-E378, wherein the second Rep-coding comprises a nucleotide sequence encoding primarily a Rep52 protein, e.g., at least 50%, 60%, 70%, 80%, 90% or more Rep52 protein relative to a Rep78 protein.
   E380. The variant baculovirus genome of any one of embodiments E362-E365 or E374-E379, wherein the second Rep-coding comprises a nucleotide sequence encoding a Rep52 protein only.
   E381. The variant baculovirus genome of any one of embodiments E362-E365 or E374-E380, wherein the second Rep-coding comprises a nucleotide sequence encoding a Rep52 protein but not a Rep78 protein.
   E382. The variant baculovirus genome of any one of embodiments E362-E365 or E374-E381, wherein:
(i) the first Rep-coding region comprises a nucleotide sequence encoding primarily a Rep78 protein, e.g., at least 50%, 60%, 70%, 80%, 90% or more Rep78 protein relative to a Rep52 protein (e.g., but not a Rep52 protein); and
(ii) the second Rep-coding region comprises a nucleotide sequence encoding a Rep52 protein but not a Rep78 protein.
   E383. The variant baculovirus genome of any one of embodiments E359, E360, or E366-E373, wherein the Rep-coding region comprises a nucleotide sequence encoding:
(i) a Rep78 protein and a Rep52 protein;
(ii) primarily a Rep78 protein, e.g., at least 50%, 60%, 70%, 80%, 90% or more Rep78 protein relative to a Rep52 protein;
(iii) a Rep78 protein only;
(iv) a Rep78 protein, but not a Rep52 protein;
(v) a Rep52 protein only; or
(vi) a Rep52 protein, but not a Rep78 protein.
   E384. The variant baculovirus genome of any one of embodiments E359, E360, E366-E373, or E383, wherein the Rep-coding region comprises a nucleotide sequence encoding a Rep78 protein and a Rep52 protein, wherein the nucleotide sequence encoding the Rep52 protein is comprised within the nucleotide sequence encoding the Rep78 protein.
   E385. The variant baculovirus genome of any one of embodiments E359, E360, E366-E373, E383, or E384, wherein the Rep-coding region comprises a single polycistronic ORF encoding a Rep78 protein and a Rep52 protein.
   E386. The variant baculovirus genome of any one of embodiments E362-E365 or E374-E382, wherein the first Rep-coding region comprises the nucleotide sequence of SEQ ID NO: 201, or a nucleotide sequence at least 70%, 75%, 80%, 85%, 90%, 95%, 96%, 97%, 98%, or 99% identical thereto; a nucleotide sequence having at least 10, 20, 50, 100, 150, 200, 250, 300, 350, 400, or 450 but no more than 500 different nucleotides relative to SEQ ID NO: 201; or a nucleotide sequence having at least 10, 20, 50, 100, 150, 200, 250, 300, 350, 400, or 450 but no more than 500 modifications (e.g., substitutions) relative to SEQ ID NO: 201.
   E387. The variant baculovirus genome of any one of embodiments E362-E365, E374-E381, or E386, wherein the first Rep-coding region encodes the amino acid sequence of SEQ ID NO: 202; an amino acid sequence at least 70%, 75%, 80%, 85%, 90%, 95%, 96%, 97%, 98%, or 99% identical thereto; an amino acid sequence comprising at least 1, 2, 3, 4, 5, 10, 15, or 20 but no more than 30 different amino acids relative to SEQ ID NO: 202; or an amino acid sequence comprising at least 1, 2, 3, 4, 5, 10, 15, or 20 but no more than 30 modifications (e.g., substitutions (e.g., conservative substitutions), insertions, or deletions) relative to the amino acid sequence of SEQ ID NO: 202.
   E388. The variant baculovirus genome of any one of embodiments E362-E365, E374-E381, E386, or E387, wherein the second Rep-coding region comprises the nucleotide sequence of SEQ ID NO: 203, or a nucleotide sequence at least 70%, 75%, 80%, 85%, 90%, 95%, 96%, 97%, 98%, or 99% identical thereto; a nucleotide sequence having at least 10, 20, 50, 100, 150, 200, 250, 300, 350, 400, or 450 but no more than 500 different nucleotides relative to SEQ ID NO: 203; or a nucleotide sequence having at least 10, 20, 50, 100, 150, 200, 250, 300, 350, 400, or 450 but no more than 500 modifications (e.g., substitutions) relative to SEQ ID NO: 203.
   E389. The variant baculovirus genome of any one of embodiments E362-E365, E374-E381, or E386-E388, wherein the second Rep-coding region encodes the amino acid sequence of SEQ ID NO: 204; an amino acid sequence at least 70%, 75%, 80%, 85%, 90%, 95%, 96%, 97%, 98%, or 99% identical thereto; an amino acid sequence comprising at least 1, 2, 3, 4, 5, 10, 15, or 20 but no more than 30 different amino acids relative to SEQ ID NO: 204; or an amino acid sequence comprising at least 1, 2, 3, 4, 5, 10, 15, or 20 but no more than 30 modifications (e.g., substitutions (e.g., conservative substitutions), insertions, or deletions) relative to SEQ ID NO: 204.
   E390. The variant baculovirus genome of any one of embodiments E362-E365, E374-E381, or E386-E389, wherein the nucleotide sequence of the first Rep-coding region, the second Rep-coding region, or both are codon optimized for an insect cell, optionally a Spodopterafrugiperda insect cell (e.g., an Sf9 insect cell).
   E391. The variant baculovirus genome of any one of embodiments E359, E360, E366-E373, or E383-E385, wherein the Rep-coding region comprises the nucleotide sequence of SEQ ID NO: 201, or a nucleotide sequence at least 70%, 75%, 80%, 85%, 90%, 95%, 96%, 97%, 98%, or 99% identical thereto; a nucleotide sequence having at least 10, 20, 50, 100, 150, 200, 250, 300, 350, 400, or 450 but no more than 500 different nucleotides relative to SEQ ID NO: 201; or a nucleotide sequence having at least 10, 20, 50, 100, 150, 200, 250, 300, 350, 400, or 450 but no more than 500 modifications (e.g., substitutions) relative to SEQ ID NO: 201.
   E392. The variant baculovirus genome of any one of embodiments E359, E360, E366-E373, or E383-E385, wherein the Rep-coding region encodes the amino acid sequence of SEQ ID NO: 202; an amino acid sequence at least 70%, 75%, 80%, 85%, 90%, 95%, 96%, 97%, 98%, or 99% identical thereto; an amino acid sequence comprising at least 1, 2, 3, 4, 5, 10, 15, or 20 but no more than 30 different amino acids relative to SEQ ID NO: 202; or an amino acid sequence comprising at least 1, 2, 3, 4, 5, 10, 15, or 20 but no more than 30 modifications (e.g., substitutions (e.g., conservative substitutions), insertions, or deletions) relative to the amino acid sequence of SEQ ID NO: 202.
   E393. The variant baculovirus genome of any one of embodiments E359, E360, E366-E373, E383-E385, E391, or E392, wherein the Rep-coding region is operably linked to a first promoter and a second promoter.
   E394. The variant baculovirus genome of any one of embodiments E359, E360, E366-E373, E383-E385, or E391-E393, wherein the first and/or second promoter is selected from a baculovirus promoter, a viral promoter, an insect viral promoter, a non-insect viral promoter, a vertebrate viral promoter, a chimeric promoter from one or more species including virus and non-virus elements, a synthetic promoter, or a variant thereof.
   E395. The variant baculovirus genome of any one of embodiments E359, E360, E366-E373, E383-E385, or E391-E394, wherein the first and/or second promoter chosen from a polh promoter, a p10 promoter, a ctx promoter, a gp64 promoter, an IE promoter, an IE-1 promoter, a p6.9 promoter, a Dmhsp70 promoter, a Hsp70 promoter, a p5 promoter, a p19 promoter, a p35 promoter, a p40 promoter, or a variant, e.g., functional fragment, thereof.
   E396. The variant baculovirus genome of any one of embodiments E359, E360, E366-E373, E383-E385, or E391-E395, wherein the first and/or second promoter is selected from an a baculovirus early promoter, baculovirus late promoter, baculovirus early-late promoter, or a baculovirus very late promoter.
   E397. The variant baculovirus genome of any one of embodiments E359, E360, E366-E373, E383-E385, or E391-E396, wherein first and/or second promoter is a baculovirus early promoter, baculovirus late promoter, or baculovirus early-late promoter.
   E398. The variant baculovirus genome of any one of embodiments E359, E360, E366-E373, E383-E385, or E391-E397, wherein first and/or second promoter is a baculovirus early-late promoter (e.g., a gp64 promoter).
   E399. The variant baculovirus genome of any one of embodiments E359, E360, E366-E373, E383-E385, or E391-E399, wherein the first or second promoter is a baculovirus very late promoter (e.g., a polh promoter).
   E400. The variant baculovirus genome of any one of embodiments E359, E360, E366-E373, E383-E385, or E391-E400, wherein:
(a) the first promoter is an baculovirus early-late promoter and the second promoter is a baculovirus very late promoter,
(b) the first promoter is a baculovirus very late promoter and the second promoter is a baculovirus early-late promoter,
(c) the first promoter is a baculovirus early promoter and the second promoter is a baculovirus early-late promoter,
(d) the first promoter is a baculovirus early-late promoter and the second promoter is a baculovirus early promoter,
(e) the first promoter is a baculovirus early promoter and the second promoter is a baculovirus late promoter,
(f) the first promoter is a baculovirus late promoter and the second promoter is a baculovirus early promoter,
(g) the first promoter is a baculovirus early-late promoter and the second promoter is a baculovirus late promoter,
(h) the first promoter is a baculovirus late promoter and the second promoter is a baculovirus early-late promoter,
(i) the first promoter is a baculovirus late promoter and the second promoter is a baculovirus very-late promoter,
(j) the first promoter is a baculovirus very-late promoter and the second promoter is a baculovirus late promoter,
(k) the first promoter is a baculovirus early promoter and the second promoter is a baculovirus very late promoter,
(l) the first promoter is a baculovirus very late promoter and the second promoter is a baculovirus early promoter,
(m) the first promoter is a baculovirus early promoter and the second promoter is a baculovirus early promoter,
(n) the first promoter is a baculovirus early-late promoter and the second promoter is a baculovirus early-late promoter, or
(o) the first promoter is a baculovirus late promoter and the second promoter is a baculovirus late promoter.
   E401. The variant baculovirus genome of any one of embodiments E359, E360, E366-E373, E383-E385, or E391-E401, wherein the first promoter is a baculovirus early-late promoter (e.g., gp64 promoter) and the second promoter is a baculovirus very late promoter (e.g., polh promoter).
   E402. The variant baculovirus genome of any one of embodiments E359, E360, E366-E373, E383-E385, or E391-E402, wherein the first promoter is a baculovirus early promoter and the second promoter is a baculovirus late promoter.
   E403. The variant baculovirus genome of any one of embodiments E396-E402, wherein
(a) the baculovirus early promoter is selected from: a lef3 promoter, a dbp promoter, a p35 promoter, an orf82 promoter, an get promoter, an orf81 promoter, an orf122 promoter, a pk-2 promoter, an orf55 promoter, an etl promoter, a hcf-1 promoter, an etm promoter, a lef-2 promoter, a lef-6 promoter, an orf84 promoter, an orf118 promoter, or an orf111 promoter,
(b) the baculovirus early-late promoter is selected from: a lef2 promoter, a orf13 promoter, a orf23 promoter, a pkip promoter, a v-fgf promoter, a pp31 promoter, an odv-e66 promoter, an orf74 promoter, an orf79 promoter, an orf82 promoter, a p15 promoter, a cg30 promoter, a helicase promoter, an he65 promoter, an orf114 promoter, a pk-2 promoter, a gp64 promoter, a gp16 promoter, an alk-exo promoter, a p35 promoter, a me53 promoter, or an ie0 promoter,
(c) the baculovirus late promoter is selected from: a ptpase promoter, an Ac-bro promoter, a ctx promoter, an orf5 promoter, an orf19 promoter, an orf20 promoter, an sod promoter, a HisP promoter, an orf34 promoter, a v-ubi promoter, an orf38 promoter, an orf43 promoter, an orf44 promoter, an orf56 promoter, an orf59 promoter, an orf60 promoter, or an fp-25k promoter, and/or
(d) the baculovirus very late promoter is selected from a p10 promoter or a polh promoter.
   E404. The variant baculovirus genome of any one of embodiments E359, E360, E366-E373, E383-E385, or E391-E403, wherein the first and/or second promoter comprises a TATA box motif and/or a CAGT motif.
   E405. The variant baculovirus genome of any one of embodiments E359, E360, E366-E373, E383-E385, or E391-E404, wherein the first and/or second promoter comprises a TAAG motif (e.g., an ATAAG nucleotide sequence).
   E406. The variant baculovirus genome of any one of embodiments E359, E360, E366-E373, E383-E385, or E391-E405, wherein the first and/or second promoter comprises both a TATA box motif and a TAAG motif.
   E407. The variant baculovirus genome of any one of embodiments E359, E360, E366-E373, E383-E385, or E391-E406, wherein the first or second promoter comprises a binding site for VLF-1.
   E408. The variant baculovirus genome of any one of embodiments E359, E360, E366-E373, E383-E385, or E391-E407, wherein the first or second promoter is a gp64 promoter (e.g., an OpMNPV gp64 promoter).
   E409. The variant baculovirus genome of any one of embodiments E359, E360, E366-E373, E383-E385, or E391-E408, wherein the first or second promoter is a polh promoter (e.g., an OpMNPV polh promoter or an AcMNPV polh promoter).
   E410. The variant baculovirus genome of any one of embodiments E359, E360, E366-E373, E383-E385, or E391-E409, wherein the first promoter is a gp64 promoter and the second promoter is a polh promoter, or wherein the first promoter is a polh promoter and the second promoter is a gp64 promoter.
   E411. The variant baculovirus genome of any one of embodiments E359, E360, E366-E373, E383-E385, or E391-E410, wherein the Rep-coding region is operably linked to a first promoter which is a baculovirus early-late promoter and a second promoter which is baculovirus very late promoter, e.g., a gp64 promoter and a polh promoter, optionally, wherein the Rep-coding region is present downstream of a homologous repeat region hr5.
   E412. The variant baculovirus genome of any one of embodiments E362-E365, E374-E381, or E386-E390, wherein the nucleotide sequence of the first Rep-coding region is operably linked to a first promoter.
   E413. The variant baculovirus genome of any one of embodiments E362-E365, E374-E381, E386-E390, or E412, wherein the nucleotide sequence of the second Rep-coding region is operably linked to a second promoter.
   E414. The variant baculovirus genome of embodiment E412 or E413, wherein the first promoter, the second promoter, or both the first promoter and the second promoter is a baculovirus major late promoter, a baculovirus early-late promoter, a baculovirus very late promoter, a viral promoter, an insect viral promoter, a non-insect viral promoter, a vertebrate viral promoter, a chimeric promoter from one or more species including virus and non-virus elements, a synthetic promoter, or a variant thereof.
   E415. The variant baculovirus genome of any one of embodiments E412-E414, wherein the first promoter, the second promoter, or both the first promoter and the second promoter is chosen from a polyhedrin (polh) promoter, a p10 promoter, a conotoxin (ctx) promoter, a gp64 promoter, an IE promoter, an IE-1 promoter, a p6.9 promoter, a Dmhsp70 promoter, a Hsp70 promoter, a p5 promoter, a p19 promoter, a p35 promoter, a p40 promoter, or a variant, e.g., functional fragment, thereof.
   E416. The variant baculovirus genome of embodiment E412-E415, wherein the first promoter and the second promoter are the same.
   E417. The variant baculovirus genome of embodiment E412-E415, wherein the first promoter and the second promoter are different.
   E418. The variant baculovirus genome of embodiment E412-E417, the first promoter and the second promoter are each a polh promoter.
   E419. The variant baculovirus genome of any one of embodiments E359, E360, E366-E373, E383-E385, or E391-E411, wherein the first promoter is a gp64 promoter and the second promoter is a polh promoter.
   E420. The variant baculovirus genome of embodiment E419, wherein the gp64 promoter comprises the nucleotide sequence of SEQ ID NO: 217; a nucleotide sequence at least 70%, 75%, 80%, 85%, 90%, 95%, 96%, 97%, 98%, or 99% identical thereto; a nucleotide sequence comprising at least one, two, three, four, five, six, or seven, but no more than ten different nucleotides relative to SEQ ID NO: 217; or a nucleotide sequence comprising at least one, two, three, four, five, six, or seven, but no more than ten modifications (e.g., substitutions) relative to SEQ ID NO: 217.
   E421. The variant baculovirus genome of embodiment E418-E420, wherein the polh promoter comprises the nucleotide sequence of SEQ ID NO: 167 or 220; a nucleotide sequence at least 70%, 75%, 80%, 85%, 90%, 95%, 96%, 97%, 98%, or 99% identical thereto; a nucleotide sequence comprising at least one, two, three, four, five, six, or seven, but no more than ten different nucleotides relative to SEQ ID NO: 167 or 220; or a nucleotide sequence comprising at least one, two, three, four, five, six, or seven, but no more than ten modifications (e.g., substitutions) relative to SEQ ID NO: 167 or 220.
   E422. The variant baculovirus genome of any one of embodiments E417 or E419-E421, wherein the first promoter and the second promoter comprises the nucleotide sequence of SEQ ID NO: 221; a nucleotide sequence at least 70%, 75%, 80%, 85%, 90%, 95%, 96%, 97%, 98%, or 99% identical thereto; a nucleotide sequence comprising at least one, two, three, four, five, six, or seven, but no more than ten different nucleotides relative to SEQ ID NO: 221; or a nucleotide sequence comprising at least one, two, three, four, five, six, or seven, but no more than ten modifications (e.g., substitutions) relative to SEQ ID NO: 221.
   E423. The variant baculovirus genome of any one of embodiments E362-E365, E374-E381, E386-E390, or E412-418, wherein the first Rep-coding region or the second Rep-coding region comprises an expression-modifier sequence which decreases transcription initiation of the first Rep-coding region.
   E424. The variant baculovirus genome of embodiment E423, wherein the expression-modifier sequence comprises a minicistron sequence.
   E425. The variant baculovirus genome of any one of embodiments E362-E365, E374-E381, E386-E390, E412-418, E423, or E424, the first Rep-coding region comprises a minicistron sequence, optionally wherein the minicistron sequence is present at the 5′ end of the first Rep-coding region.
   E426. The variant baculovirus genome of any one of embodiments E423-E425, the first Rep-coding region comprises between 3-100 nucleotides between the expression-modifier sequence and the start codon of the first ORF; optionally between 3-25 nucleotides, between 3-10 nucleotides, or 3 nucleotides between the expression-modifier sequence and the start codon of the first ORF.
   E427. The variant baculovirus genome of any one of embodiments E423-E426, wherein the minicistron sequence is from a baculovirus gene; optionally a baculovirus gp64 gene.
   E428. The variant baculovirus genome of any one of embodiments E423-E427, wherein the minicistron sequence comprises SEQ ID NO: 9 or SEQ ID NO: 10; a nucleotide sequence at least 70%, 75%, 80%, 85%, 90%, 95%, 96%, 97%, 98%, or 99% identical to SEQ ID NO: 9 or 10; a nucleotide sequence comprising one, two, or three modifications (e.g., substitutions), but no more than four modifications (e.g., substitutions) relative to SEQ ID NO: 9 or 10; or a nucleotide sequence comprising one, two, or three, but no more than four different nucleotides relative to SEQ ID NO: 9 or 10.
   E429. The variant baculovirus genome of any one of embodiments E362-E365, E374-E381, E386-E390, E412-418, or E423-E428, which comprises in 5′ to 3′ order: a polh promoter, a minicistron sequence, and the first Rep-coding region comprising a nucleotide sequence encoding primarily a Rep78 protein, e.g., at least 50%, 60%, 70%, 80%, 90% or more Rep78 protein relative to a Rep52 protein (e.g., but not a Rep52 protein).
   E430. The variant baculovirus genome of any one of embodiments E362-E365, E374-E381, E386-E390, E412-418, or E423-E429, which comprises in 5′ to 3′ order: a polh promoter, and the first Rep-coding region comprising a nucleotide sequence encoding primarily a Rep78 protein, e.g., at least 50%, 60%, 70%, 80%, 90% or more Rep78 protein relative to a Rep52 protein (e.g., but not a Rep52 protein).
   E431. The variant baculovirus genome of any one of embodiments E362-E365, E374-E381, E386-E390, E412-418, or E423-E430, which comprises in 5′ to 3′ order: a polh promoter and the second Rep-coding region comprising a nucleotide sequence encoding a Rep52 protein but not a Rep78 protein.
   E432. The variant baculovirus genome of any one of embodiments E362-E365, E374-E381, E386-E390, E412-418, or E423-E431, which comprises:
(i) in 5′ to 3′ order: a polh promoter, a minicistron sequence, and the first Rep-coding region comprising a nucleotide sequence encoding primarily a Rep78 protein, e.g., at least 50%, 60%, 70%, 80%, 90% or more Rep78 protein relative to a Rep52 protein (e.g., but not a Rep52 protein); and
(ii) in 5′ to 3′ order: a polh promoter and the second Rep-coding region comprising a nucleotide sequence encoding a Rep52 protein but not a Rep78 protein.
   E433. The variant baculovirus genome of any one of embodiments E362-E365, E374-E381, E386-E390, E412-418, E423-E432, wherein:
(i) in 5′ to 3′ order: a polh promoter and the first Rep-coding region comprising a nucleotide sequence encoding primarily a Rep78 protein, e.g., at least 50%, 60%, 70%, 80%, 90% or more Rep78 protein relative to a Rep52 protein (e.g., but not a Rep52 protein); and
(ii) in 5′ to 3′ order: a polh promoter and the second Rep-coding region comprising a nucleotide sequence encoding a Rep52 protein but not a Rep78 protein.
   E434. The variant baculovirus genome of any one of embodiments E359, E360, E366-E373, E383-E385, or E391-E422, which comprises in 5′ to 3′ order: a first promoter, a second promoter, and the Rep-coding region comprising a nucleotide sequence encoding a Rep78 protein and Rep52 protein.
   E435. The variant baculovirus genome of any one of embodiments E359, E360, E366-E373, E383-E385, E391-E422, or E434, which comprises in 5′ to 3′ order: a baculovirus early-late promoter, a baculovirus very late promoter, and the Rep-coding region comprising a nucleotide sequence encoding a Rep78 protein and Rep52 protein.
   E436. The variant baculovirus genome of any one of embodiments E359, E360, E366-E373, E383-E385, E391-E422, E434, or E435, which comprises in 5′ to 3′ order: a g64 promoter, a polh promoter, and the Rep-coding region comprising a nucleotide sequence encoding a Rep78 protein and Rep52 protein.
   E437. The variant baculovirus genome of any one of embodiments E362-E365, E374-E381, E386-E390, E412-418, or E423-E433, wherein:
(i) the first Rep-coding region is present in first location in the variant baculovirus genome chosen from ChiA, v-cath, p10, egt, polyhedrin, SOD, ctx, p26, odv-e56, p74 (PIF0), PIF1, PIF2, PIF3, PIF4, PIF5, Tn7, AcORF-91, AcORF-108, AcORF-52, v-ubi, or p94; and
(ii) the second Rep-coding region is present in a second location in the variant baculovirus genome chosen from ChiA, v-cath, p10, egt, polyhedrin, SOD, ctx, p26, odv-e56, p74 (PIF0), PIF1, PIF2, PIF3, PIF4, PIF5, Tn7, AcORF-91, AcORF-108, AcORF-52, v-ubi, or p94;
wherein the first locus and the second locus are different.
   E438. The variant baculovirus genome of embodiment E362-E365, E374-E381, E386-E390, E412-418, E423-E433, or E437, wherein the first Rep-coding region is present in v-cath locus and the second Rep-coding region is present in the egt locus.
   E439. The variant baculovirus genome of embodiment E362-E365, E374-E381, E386-E390, E412-418, E423-E433, E437, or E438, wherein the first Rep-coding region is present in Tn7/polh locus and the second Rep-coding region is present in the egt locus.
   E440. The variant baculovirus genome of any one of embodiments E362-E365, E374-E381, E386-E390, E412-418, E423-E433, E437, or E438, wherein the first Rep-coding region is present in the v-cath locus of the variant baculovirus genome and is operably linked to a polh promoter, and the second Rep-coding region is present in the egt locus of the variant baculovirus genome and is operably linked to a polh promoter.
   E441. The variant baculovirus genome of any one of embodiments E362-E365, E374-E381, E386-E390, E412-418, E423-E433, E437, or E440, wherein:
(i) the first Rep-coding region comprises a nucleotide sequence encoding primarily a Rep78 protein, e.g., at least 50%, 60%, 70%, 80%, 90% or more Rep78 protein relative to a Rep52 protein (e.g., but not a Rep52 protein), wherein the first Rep-coding region is present in the v-cath locus of the variant baculovirus genome; and
(ii) the second Rep-coding region comprises a nucleotide sequence encoding a Rep52 protein but not a Rep78 protein, wherein the second Rep-coding region is present in the egt locus of the variant baculovirus genome.
   E442. The variant baculovirus genome of any one of embodiments E362-E365, E374-E381, E386-E390, E412-418, E423-E433, E437, E440, or E441, wherein:
(i) the first Rep-coding region comprises a nucleotide sequence encoding primarily a Rep78 protein, e.g., at least 50%, 60%, 70%, 80%, 90% or more Rep78 protein relative to a Rep52 protein (e.g., but not a Rep52 protein), wherein the first Rep-coding region is present in the v-cath locus of the variant baculovirus genome and is operably linked to a polh promoter; and
(ii) the second Rep-coding region comprises a nucleotide sequence encoding a Rep52 protein but not a Rep78 protein, wherein the second Rep-coding region is present in the egt locus of the variant baculovirus genome and is operably linked to a polh promoter.
   E443. The variant baculovirus genome of any one of embodiments E362-E365, E374-E381, E386-E390, E412-418, E423-E433, E437, or E440-E442, which comprises:
(i) in 5′ to 3′ order a polh promoter, a minicistron sequence, and the first Rep-coding region comprising a nucleotide sequence encoding primarily a Rep78 protein, e.g., at least 50%, 60%, 70%, 80%, 90% or more Rep78 protein relative to a Rep52 protein (e.g., but not a Rep52 protein), wherein the first Rep-coding region is present in the v-cath locus of the variant baculovirus genome; and
(ii) in 5′ to 3′ order: a polh promoter and the second Rep-coding region comprising a nucleotide sequence encoding a Rep52 protein but not a Rep78 protein, wherein the second Rep-coding region is present in the egt locus of the variant baculovirus genome.
   E444. The variant baculovirus genome of any one of embodiments E362-E365, E374-E381, E386-E390, E412-418, E423-E433, E437, or E440-E443, which comprises:
(i) in 5′ to 3′ order: a polh promoter and the first Rep-coding region comprising a nucleotide sequence encoding primarily a Rep78 protein, e.g., at least 50%, 60%, 70%, 80%, 90% or more Rep78 protein relative to a Rep52 protein (e.g., but not a Rep52 protein), wherein the first Rep-coding region is present in the v-cath locus of the variant baculovirus genome; and
(ii) in 5′ to 3′ order: a polh promoter and the second Rep-coding region comprising a nucleotide sequence encoding a Rep52 protein but not a Rep78 protein, wherein the second Rep-coding region is present in the egt locus of the variant baculovirus genome.
   E445. The variant baculovirus genome of any one of embodiments E359, E360, E366-E373, E383-E385, E391-E422, or E434-E436, wherein: the Rep-coding region is present in a location in the variant baculovirus genome chosen from chiA, v-cath, p10, egt, polyhedrin, SOD, ctx, p26, odv-e56, p74 (PIF0), PIF1, PIF2, PIF3, PIF4, PIF5, Tn7, AcORF-91, AcORF-108, AcORF-52, v-ubi, or p94.
   E446. The variant baculovirus genome of any one of embodiments E359, E360, E366-E373, E383-E385, E391-E422, E434-E436, or E445, wherein the Rep-coding region is present in the p74 locus of the variant baculovirus genome.
   E447. The variant baculovirus genome of any one of embodiments E359, E360, E366-E373, E383-E385, E391-E422, E434-E436, E445, or E446, wherein the Rep-coding region is present downstream of a homologous repeat region hr5.
   E448. The variant baculovirus genome of any one of embodiments E359, E360, E366-E373, E383-E385, E391-E422, E434-E436, or E445-E447, wherein the Rep-coding region is present in the p74 locus of the variant baculovirus genome and is present downstream of a homologous repeat region hr5.
   E449. The variant baculovirus genome of any one of embodiments E359, E360, E366-E373, E383-E385, E391-E422, E434-E436, or E445-E448, wherein the Rep-coding region is present in the p74 locus of the variant baculovirus genome, and is operably linked to a gp64 promoter and a polh promoter.
   E450. The variant baculovirus genome of any one of embodiments E359, E360, E366-E373, E383-E385, E391-E422, E434-E436, or E445-E449, wherein the Rep-coding region is present in the p74 locus of the variant baculovirus genome and wherein the Rep-coding region comprises a single polycistronic ORF encoding a Rep78 protein and a Rep52 protein.
   E451. The variant baculovirus genome of any one of embodiments E359, E360, E366-E373, E383-E385, E391-E422, E434-E436, or E445-E450, wherein the Rep-coding region is present in the p74 locus of the variant baculovirus genome and wherein the Rep-coding region comprises a nucleotide sequence encoding a Rep78 protein and a Rep52 protein, wherein the nucleotide sequence encoding the Rep52 protein is comprised within the nucleotide sequence encoding the Rep78 protein.
   E452. The variant baculovirus genome of any one of embodiments E359, E360, E366-E373, E383-E385, E391-E422, E434-E436, or E445-E451, wherein the Rep-coding region is present in the p74 gene locus of the variant baculovirus genome and wherein the Rep-coding region comprises:
(i) a nucleotide sequence encoding a Rep78 protein and a Rep52 protein, wherein the nucleotide sequence encoding the Rep52 protein is comprised within the nucleotide sequence encoding the Rep78 protein; and
(ii) is operably linked to a gp64 promoter and a polh promoter, optionally wherein:
   (a) the gp64 promoter comprises the nucleotide sequence of SEQ ID NO: 217; a nucleotide sequence at least 70%, 75%, 80%, 85%, 90%, 95%, 96%, 97%, 98%, or 99% identical thereto; a nucleotide sequence comprising at least one, two, three, four, five, six, or seven, but no more than ten different nucleotides relative to SEQ ID NO: 217; or a nucleotide sequence comprising at least one, two, three, four, five, six, or seven, but no more than ten modifications (e.g., substitutions) relative to SEQ ID NO: 217; and/or
   (b) the polh promoter comprises the nucleotide sequence of SEQ ID NO: 167 or 220; a nucleotide sequence at least 70%, 75%, 80%, 85%, 90%, 95%, 96%, 97%, 98%, or 99% identical thereto; a nucleotide sequence comprising at least one, two, three, four, five, six, or seven, but no more than ten different nucleotides relative to SEQ ID NO: 167 or 220; or a nucleotide sequence comprising at least one, two, three, four, five, six, or seven, but no more than ten modifications (e.g., substitutions) relative to SEQ ID NO: 167 or 220.
      E453. The variant baculovirus genome of any one of embodiments E359, E360, E366-E373, E383-E385, E391-E422, E434-E436, or E445-E452, wherein the Rep-coding region is present in the p74 gene locus of the variant baculovirus genome and wherein the Rep-coding region comprises:
(i) a single polycistronic ORF encoding a Rep78 protein and a Rep52 protein, wherein the ORF encoding the Rep78 protein comprises an ATG or a CTG start codon; and
(ii) is operably linker to a gp64 promoter and a polh promoter, optionally wherein:
   (a) the gp64 promoter comprises the nucleotide sequence of SEQ ID NO: 217; a nucleotide sequence at least 70%, 75%, 80%, 85%, 90%, 95%, 96%, 97%, 98%, or 99% identical thereto; a nucleotide sequence comprising at least one, two, three, four, five, six, or seven, but no more than ten different nucleotides relative to SEQ ID NO: 217; or a nucleotide sequence comprising at least one, two, three, four, five, six, or seven, but no more than ten modifications (e.g., substitutions) relative to SEQ ID NO: 217; and or
   (b) the polh promoter comprises the nucleotide sequence of SEQ ID NO: 167 or 220; a nucleotide sequence at least 70%, 75%, 80%, 85%, 90%, 95%, 96%, 97%, 98%, or 99% identical thereto; a nucleotide sequence comprising at least one, two, three, four, five, six, or seven, but no more than ten different nucleotides relative to SEQ ID NO: 167 or 220; or a nucleotide sequence comprising at least one, two, three, four, five, six, or seven, but no more than ten modifications (e.g., substitutions) relative to SEQ ID NO: 167 or 220.
      E454. The variant baculovirus genome of any one of embodiments E359, E360, E366-E373, E383-E385, E391-E422, E434-E436, or E445-E453, which comprises in 5′ to 3′ order, a gp64 promoter, a polh promoter, and the Rep-coding region comprising a nucleotide sequence encoding a Rep78 protein and a Rep52 protein, wherein the nucleotide sequence encoding the Rep52 protein is comprised within the nucleotide sequence encoding the Rep78 protein, wherein the Rep-coding region is present in the p74 gene locus of the variant baculovirus genome, optionally wherein the Rep-coding region is present downstream of a homologous repeat region hr5.
      E455. The variant baculovirus genome of any one of embodiments E359, E360, E366-E373, E383-E385, E391-E422, E434-E436, or E445-E454, which comprises in 5′ to 3′ order, a gp64 promoter, a polh promoter, and the Rep-coding region comprising a nucleotide sequence comprising a single polycistronic ORF encoding a Rep78 protein and a Rep52 protein, wherein the ORF encoding the Rep78 protein comprises an ATG or a CTG start codon, wherein the Rep-coding region is present in the p74 gene locus, optionally wherein the Rep-coding region is present downstream of a homologous repeat region hr5.
      E456. The variant baculovirus genome of any one of embodiments E292-E455, wherein the VP-coding region comprises a nucleotide sequence encoding:
(i) primarily a VP1 protein, e.g., at least 50%, 60%, 70%, 80%, 90% or more VP1 protein relative to a VP2 protein and/or a VP3 protein;
(ii) a VP1 protein only;
(iii) a VP1 protein, but not a VP2 protein or a VP3 protein;
(iv) primarily a VP2 protein, e.g., at least about 50%, 60%, 70%, 80%, 90% or more VP2 protein relative to a VP1 protein and/or a VP3 protein;
(v) a VP2 protein only;
(vi) a VP2 protein, but not a VP1 protein or a VP3 protein;
(vii) a VP3 protein only;
(viii) a VP3 protein, but not a VP1 protein or a VP2 protein;
(ix) a VP1 protein and a VP2 protein, but not a VP3 protein;
(x) a VP1 protein and a VP3 protein, but not a VP2 protein;
(xi) a VP2 protein and a VP3 protein, but not a VP1 protein;
(xii) a VP1 protein, a VP2 protein, and a VP3 protein.
   E457. The variant baculovirus genome of any one of embodiments E292-E456, wherein the VP-coding region comprises a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein.
   E458. The variant baculovirus genome of any one of embodiments E292-E457, wherein the VP-coding region comprises a single polycistronic ORF encoding a VP1 protein, a VP2 protein, and a VP3 protein.
   E459. The variant baculovirus genome of any one of embodiments E292-E458, wherein the ORF encoding the VP1 protein comprises an ACG start codon, the ORF encoding the VP2 protein comprises an ACG start codon, and the ORF encoding the VP3 protein comprises an ATG start codon.
   E460. The variant baculovirus genome of any one of embodiments E292-E459, wherein the ORF encoding the VP1 protein comprises an ATG start codon, the ORF encoding the VP2 protein comprises an ACG start codon, and the ORF encoding the VP3 protein comprises an ATG start codon.
   E461. The variant baculovirus genome of any one of embodiments E292-E460, wherein the VP-coding region encodes an AAV1 capsid protein, an AAV2 capsid protein, an AAV3 capsid protein, an AAV4 capsid protein, an AAV5 capsid protein, an AAV6 capsid protein, an AAV8 capsid protein, an AAV9 capsid protein, an AAVrh10 capsid protein or a variant of any of the aforesaid capsid proteins.
   E462. The variant baculovirus genome of any one of embodiments E292-E461, wherein the VP-coding region encodes an AAV5 capsid protein or variant thereof, or an AAV9 capsid protein or variant thereof.
   E463. The variant baculovirus genome of any one of embodiments E292-E462, wherein the VP-coding region encodes a VP1 protein comprising the amino acid sequence of any of SEQ ID NOs: 46, 47, 71, or 168, or an amino acid sequence at least 70%, 75%, 80%, 85%, 90%, 95%, 96%, 97%, 98%, or 99% identical to any of the aforesaid amino acid sequences.
   E464. The variant baculovirus genome of any one of embodiments E292-E463, wherein the VP-coding region encodes a VP2 protein e.g., a fragment or a portion, of any of SEQ ID NOs: 46, 47, 71, or 168, or an amino acid sequence at least 70%, 75%, 80%, 85%, 90%, 95%, 96%, 97%, 98%, or 99% identical to any of the aforesaid amino acid sequences, optionally wherein the VP2 protein comprises amino acids 138-736 or SEQ ID NOs:46, 47, or 71, or amino acids 137-724 of SEQ ID NO: 168.
   E465. The variant baculovirus genome of any one of embodiments E292-E464, wherein the VP-coding region encodes a VP3 protein e.g., a fragment or a portion, of any of SEQ ID NOs:46, 47, 71, or 168, or an amino acid sequence at least 70%, 75%, 80%, 85%, 90%, 95%, 96%, 97%, 98%, or 99% identical to any of the aforesaid amino acid sequences, optionally wherein the VP3 protein comprises amino acids 203-736 of SEQ ID NOs: 46, 47, or 71, or amino acids 193-724 of SEQ ID NO: 168.
   E466. The variant baculovirus genome of any one of embodiments E292-E465, wherein the VP-coding region comprises the nucleotide sequence of any of SEQ ID NOs: 43-45, 72, 169, 205, 212, or 213, or a nucleotide sequence at least 70%, 75%, 80%, 85%, 90%, 95%, 96%, 97%, 98%, or 99% identical to any of the aforesaid nucleotide sequences.
   E467. The variant baculovirus genome of any one of embodiments E292-E466, wherein the VP-coding region comprises a nucleotide sequence encoding a VP2 protein e.g., a fragment or a portion, of any of SEQ ID NOs:43-45, 72, 169, 205, 212, or 213, or a nucleotide sequence at least 70%, 75%, 80%, 85%, 90%, 95%, 96%, 97%, 98%, or 99% identical to any of the aforesaid nucleotide sequences, optionally wherein the nucleotide sequence encoding the VP2 protein comprises nucleotides 412-2211 of SEQ ID NOs:43-45, 72, 205, or 212, or nucleotides 409-2175 of SEQ ID NO: 169 or 213.
   E468. The variant baculovirus genome of any one of embodiments E292-E467, wherein the VP-coding region comprises a nucleotide sequence encoding a VP3 protein e.g., a fragment or a portion, of any of SEQ ID NOs:43-45, 72, 169, 205, 212, or 213, or a nucleotide sequence at least 70%, 75%, 80%, 85%, 90%, 95%, 96%, 97%, 98%, or 99% identical to any of the aforesaid nucleotide sequences, optionally wherein the nucleotide sequence encoding the VP3 protein comprises nucleotides 607-2211 of SEQ ID NOs: 43-45, 72, 205, or 212, or nucleotides 577-2175 of SEQ ID NO: 169 or 213.
   E469. The variant baculovirus genome of any one of embodiments E292-E468, wherein the nucleotide sequence of the VP-coding region is codon optimized for an insect cell, optionally a Spodoptera frugiperda insect cell (e.g., an Sf9 insect cell).
   E470. The variant baculovirus genome of any one of embodiments E292-E469, wherein nucleotide sequence of the VP-coding region is operably linked to a promoter.
   E471. The variant baculovirus genome of embodiment E470, wherein the promoter is a baculovirus major late promoter, a viral promoter, an insect viral promoter, a non-insect viral promoter, a vertebrate viral promoter, a chimeric promoter from one or more species including virus and non-virus elements, a synthetic promoter, or a variant thereof.
   E472. The variant baculovirus genome of embodiment E470 or E471, wherein the promoter is chosen from a polh promoter, a p10 promoter, a ctx promoter, a gp64 promoter, an IE promoter, an IE-1 promoter, a p6.9 promoter, a Dmhsp70 promoter, a Hsp70 promoter, a p5 promoter, a p19 promoter, a p35 promoter, a p40 promoter, or a variant, e.g., functional fragment, thereof.
   E473. The variant baculovirus genome of any one of embodiments E470-E472, wherein the promoter is a p10 promoter.
   E474. The variant baculovirus genome of embodiment E473, wherein the p10 promoter comprises the nucleotide sequence of SEQ ID NO: 200; a nucleotide sequence at least 70%, 75%, 80%, 85%, 90%, 95%, 96%, 97%, 98%, or 99% identical thereto; a nucleotide sequence comprising at least one, two, three, four, five, six, or seven, but no more than ten different nucleotides relative to SEQ ID NO: 200; or a nucleotide sequence comprising at least one, two, three, four, five, six, or seven, but no more than ten modifications (e.g., substitutions) relative to SEQ ID NO: 200.
   E475. The variant baculovirus genome of any one embodiments E292-E474, which comprises in 5′ to 3′ order, a p10 promoter and the VP-coding region comprising a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein.
   E476. The variant baculovirus genome of any one of embodiments E362-E365, E374-E381, E386-E390, E412-418, E423-E433, E437, E440-E444, or E456-E475, wherein the VP-coding region is present in a different location in the variant baculovirus genome than the location of the first Rep-coding region, the second Rep-coding region or both.
   E477. The variant baculovirus genome of any one of embodiments E362-E365, E374-E381, E386-E390, E412-418, E423-E433, E437, E440-E444, or E456-E475, wherein the VP-coding region is present in the same location of the variant baculovirus genome as the first Rep-coding region.
   E478. The variant baculovirus genome of any one of embodiments E362-E365, E374-E381, E386-E390, E412-418, E423-E433, E437, E440-E444, E456-E475, or E477, wherein the VP-coding region is present in the reverse orientation relative to the first Rep-coding region.
   E479. The variant baculovirus genome of any one of embodiments E362-E365, E374-E381, E386-E390, E412-418, E423-E433, E437, E440-E444, E456-E475, E477, or E478, wherein the VP-coding region is present in the same location of the variant baculovirus genome as the first Rep-coding region and is present in the reverse orientation relative to the first Rep-coding region.
   E480. The variant baculovirus genome of any one of embodiments E292-E479, wherein the VP-coding region is present in a location in variant baculovirus genome chosen from ChiA, v-cath, p10, egt, polyhedrin, SOD, ctx, p26, odv-e56, p74 (PIF0), PIF1, PIF2, PIF3, PIF4, PIF5, Tn7, AcORF-91, AcORF-108, AcORF-52, v-ubi, or p94.
   E481. The variant baculovirus genome of any one of embodiments E292-E480, wherein the VP-coding region is present in the v-cath gene locus of the variant baculovirus genome.
   E482. The variant baculovirus genome of any one of embodiments E292-E480, wherein the VP-coding region is present in the Tn7/polh gene locus of the variant baculovirus genome.
   E483. The variant baculovirus genome of any one of embodiments E292-E480, wherein the VP-coding region is present in the SOD gene locus of the variant baculovirus genome.
   E484. The variant baculovirus genome of any one of embodiments E292-E481, wherein the VP-coding region is present in the v-cath gene locus of the variant baculovirus genome and wherein the VP-coding region is operably linked to a p10 promoter.
   E485. The variant baculovirus genome of any one of embodiments E292-E480 or E483, wherein the VP-coding region is present in the SOD gene locus of the variant baculovirus genome and wherein the VP-coding region is operably linked to a p10 promoter.
   E486. The variant baculovirus genome of any one of embodiments E292-E481 or E484, wherein the VP-coding region is present in the v-cath gene locus of the variant baculovirus genome and wherein the VP-coding region comprises a single polycistronic ORF encoding a VP1 protein, a VP2 protein, and a VP3 protein.
   E487. The variant baculovirus genome of any one of embodiments E292-E480, E483, or E485, wherein the VP-coding region is present in the SOD gene locus of the variant baculovirus genome and wherein the VP-coding region comprises a single polycistronic ORF encoding a VP1 protein, a VP2 protein, and a VP3 protein.
   E488. The variant baculovirus genome of any one of embodiments E292-E481 E484, or E486, wherein the VP-coding region is present in the v-cath gene locus of the variant baculovirus genome and wherein the VP-coding region comprises a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein.
   E489. The variant baculovirus genome of any one of embodiments E292-E480, E483, E485, or E487, wherein the VP-coding region is present in the SOD gene locus of the variant baculovirus genome and wherein the VP-coding region comprises a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein.
   E490. The variant baculovirus genome of any one of embodiments E292-E481 E484, E486, or E488, wherein the VP-coding region is present in the v-cath gene locus of the variant baculovirus genome and wherein the VP-coding region:
(i) comprises a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein; and
(ii) is operably linked to a p10 promoter.
   E491. The variant baculovirus genome of any one of embodiments E292-E480, E483, E485, E487, or E489, wherein the VP-coding region is present in the SOD gene locus of the variant baculovirus genome and wherein the VP-coding region:
(i) comprises a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein; and
(ii) is operably linked to a p10 promoter.
   E492. The variant baculovirus genome of any one of embodiments E292-E481 E484, E486, E488, or E490, which comprises in 5′ to 3′ order, a p10 promoter and the VP-coding region comprising a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein, wherein the VP-coding region is present in the v-cath gene locus of the variant baculovirus genome.
   E493. The variant baculovirus genome of any one of embodiments E292-E480, E483, E485, E487, E489, or E491, which comprises in 5′ to 3′ order, a p10 promoter and the VP-coding region comprising a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein, wherein the VP-coding region is present in the SOD gene locus of the variant baculovirus genome.
   E494. The variant baculovirus genome of any one of embodiments E292-E481 E484, E486, E488, E490, or E492, wherein the VP-coding region is present in the v-cath gene locus of the variant baculovirus genome and wherein the VP-coding region:
(i) comprises a single polycistronic ORF encoding a VP1 protein, a VP2 protein, and a VP3 protein, wherein the ORF encoding the VP1 protein comprises an ACG start codon, the ORF encoding the VP2 protein comprises an ACG start codon, and the ORF encoding the VP3 protein comprises an ATG start codon; and
(ii) is operably linked to a p10 promoter.
   E495. The variant baculovirus genome of any one of embodiments E292-E480, E483, E485, E487, E489, E491, or E493, wherein the VP-coding region is present in the SOD gene locus of the variant baculovirus genome and wherein the VP-coding region:
(i) comprises a single polycistronic ORF encoding a VP1 protein, a VP2 protein, and a VP3 protein, wherein the ORF encoding the VP1 protein comprises an ACG start codon, the ORF encoding the VP2 protein comprises an ACG start codon, and the ORF encoding the VP3 protein comprises an ATG start codon; and
(ii) is operably linked to a p10 promoter.
   E496. The variant baculovirus genome of any one of embodiments E292-E481 E484, E486, E488, E490, E492, or E494, wherein the VP-coding region is present in the v-cath gene locus of the variant baculovirus genome and wherein the VP-coding region:
(i) comprises a single polycistronic ORF encoding a VP1 protein, a VP2 protein, and a VP3 protein, wherein the ORF encoding the VP1 protein comprises an ATG start codon, the ORF encoding the VP2 protein comprises an ACG start codon, and the ORF encoding the VP3 protein comprises an ATG start codon; and
(ii) is operably linked to a p10 promoter.
   E497. The variant baculovirus genome of any one of embodiments E292-E480, E483, E485, E487, E489, E491, E493, or E495, wherein the VP-coding region is present in the SOD gene locus of the variant baculovirus genome and wherein the VP-coding region:
(i) comprises a single polycistronic ORF encoding a VP1 protein, a VP2 protein, and a VP3 protein, wherein the ORF encoding the VP1 protein comprises an ATG start codon, the ORF encoding the VP2 protein comprises an ACG start codon, and the ORF encoding the VP3 protein comprises an ATG start codon; and
(ii) is operably linked to a p10 promoter.
   E498. The variant baculovirus genome of any one of embodiments E292-E481 E484, E486, E488, E490, E492, E494, or E496, which comprises in 5′ to 3′ order a p10 promoter; and the VP-coding region comprising a nucleotide sequence comprising a single polycistronic ORF encoding a VP1 protein, a VP2 protein, and a VP3 protein, wherein the ORF encoding the VP1 protein comprises an ACG start codon, the ORF encoding the VP2 protein comprises an ACG start codon, and the ORF encoding the VP3 protein comprises an ATG start codon, wherein the VP-coding region is present in the v-cath gene locus of the variant baculovirus genome.
   E499. The variant baculovirus genome of any one of embodiments E292-E480, E483, E485, E487, E489, E491, E493, E495, or E497, which comprises in 5′ to 3′ order a p10 promoter; and the VP-coding region comprising a nucleotide sequence comprising a single polycistronic ORF encoding a VP1 protein, a VP2 protein, and a VP3 protein, wherein the ORF encoding the VP1 protein comprises an ACG start codon, the ORF encoding the VP2 protein comprises an ACG start codon, and the ORF encoding the VP3 protein comprises an ATG start codon, wherein the VP-coding region is present in the SOD gene locus of the variant baculovirus genome.
   E500. The variant baculovirus genome of any one of embodiments E292-E481 E484, E486, E488, E490, E492, E494, E496, or E498, which comprises in 5′ to 3′ order, a p10 promoter; and the VP-coding region comprising a nucleotide sequence comprising a single polycistronic ORF encoding a VP1 protein, a VP2 protein, and a VP3 protein, wherein the ORF encoding the VP1 protein comprises an ATG start codon, the ORF encoding the VP2 protein comprises an ACG start codon, and the ORF encoding the VP3 protein comprises an ATG start codon, wherein the VP-coding region is present in the v-cath gene locus of the variant baculovirus genome.
   E501. The variant baculovirus genome of any one of embodiments E292-E480, E483, E485, E487, E489, E491, E493, E495, E497, or E499, which comprises in 5′ to 3′ order, a p10 promoter; and the VP-coding region comprising a nucleotide sequence comprising a single polycistronic ORF encoding a VP1 protein, a VP2 protein, and a VP3 protein, wherein the ORF encoding the VP1 protein comprises an ATG start codon, the ORF encoding the VP2 protein comprises an ACG start codon, and the ORF encoding the VP3 protein comprises an ATG start codon, wherein the VP-coding region is present in the SOD gene locus of the variant baculovirus genome.
   E502. The variant baculovirus genome of any one of embodiments E292-E501, which further comprises a second VP-coding region.
   E503. The variant baculovirus genome of embodiment E502, wherein the second VP-coding region comprises a nucleotide sequence encoding a VP1 protein.
   E504. The variant baculovirus genome of embodiment E502 or E503, wherein the second VP-coding region comprises a nucleotide sequence encoding primarily a VP1 protein, e.g., at least 50%, 60%, 70%, 80%, 90% or more VP1 protein relative to a VP2 protein and/or a VP3 protein.
   E505. The variant baculovirus genome of any one of embodiments E502-E504, wherein the second VP-coding region comprises a nucleotide sequence encoding a VP1 protein only.
   E506. The variant baculovirus genome of embodiment E502 or E503, wherein the second VP-coding region comprises a nucleotide sequence encoding a VP1 protein but not a VP2 protein or a VP3 protein.
   E507. The variant baculovirus genome of any one of embodiments E502-E505, wherein the second VP-coding region comprises a single ORF, comprising a start codon and a nucleotide sequence encoding primarily a VP1 protein, e.g., at least 50%, 60%, 70%, 80%, 90% or more VP1 protein relative to a VP2 protein and/or a VP3 protein.
   E508. The variant baculovirus genome of embodiment E502-E505 or E507, wherein the second VP-coding region comprises a single ORF, comprising a start codon and a nucleotide sequence encoding a VP1 protein.
   E509. The variant baculovirus genome of embodiment E502, E503, or E505, wherein the second VP-coding region comprises a single ORF, comprising a start codon and a nucleotide sequence encoding a VP1 protein, but not a VP2 protein or a VP3 protein.
   E510. The variant baculovirus genome of embodiment E507-E509, wherein the ORF comprises an ATG start codon (e.g., a canonical start codon).
   E511. The variant baculovirus genome of embodiment E507-E509, wherein the ORF comprises an ACG start codon, a CTG start codon, a TTG start codon, or GTG start codon (e.g., a non-canonical start codon).
   E512. The variant baculovirus genome of any one of embodiments E502-E511, wherein the second VP-coding region encodes an AAV1 capsid protein, an AAV2 capsid protein, an AAV3 capsid protein, an AAV4 capsid protein, an AAV5 capsid protein, an AAV6 capsid protein, an AAV8 capsid protein, an AAV9 capsid protein, an AAVrh10 capsid protein or a variant of any of the aforesaid capsid proteins.
   E513. The variant baculovirus genome of any one of embodiments E502-E512, wherein the second VP-coding region encodes an AAV5 capsid protein or variant thereof, or an AAV9 capsid protein or variant thereof.
   E514. The variant baculovirus genome of any one of embodiments E502-E513, wherein the second VP-coding region encodes a VP1 protein comprising the amino acid sequence of any of SEQ ID NOs:46, 47, 71, or 168, or an amino acid sequence at least 70%, 75%, 80%, 85%, 90%, 95%, 96%, 97%, 98%, or 99% identical to any of the aforesaid amino acid sequences.
   E515. The variant baculovirus genome of embodiment E502-E514, wherein the second VP-coding region comprises the nucleotide sequence of SEQ ID NO:43-45, 72, 169, 205, 212, or 213, or a nucleotide sequence at least 70%, 75%, 80%, 85%, 90%, 95%, 96%, 97%, 98%, or 99% identical thereto; a nucleotide sequence having at least 10, 20, 50, 100, 150, 200, 250, 300, 350, 400, or 450 but no more than 500 different nucleotides relative to SEQ ID NO:43-45, 72, 169, 205, 212, or 213; or a nucleotide sequence having at least 10, 20, 50, 100, 150, 200, 250, 300, 350, 400, or 450 but no more than 500 modifications (e.g., substitutions) relative to SEQ ID NO:43-45, 72, 169, 205, 212, or 213.
   E516. The variant baculovirus genome of any one of embodiments E502-E515, wherein the nucleotide sequence of the second VP-coding region is codon optimized for an insect cell, optionally a Spodoptera frugiperda insect cell (e.g., an Sf9 insect cell).
   E517. The variant baculovirus genome of any one of embodiments E502-E516, wherein nucleotide sequence of the second VP-coding region is operably linked to a promoter.
   E518. The variant baculovirus genome of embodiment E517, wherein the promoter is a baculovirus major late promoter, a viral promoter, an insect viral promoter, a non-insect viral promoter, a vertebrate viral promoter, a chimeric promoter from one or more species including virus and non-virus elements, a synthetic promoter, or a variant thereof.
   E519. The variant baculovirus genome of embodiment E517 or E518, wherein the promoter is chosen from a polh promoter, a p10 promoter, a ctx promoter, a gp64 promoter an IE promoter, an IE-1 promoter, a p6.9 promoter, a Dmhsp70 promoter, a Hsp70 promoter, a p5 promoter, a p19 promoter, a p35 promoter, a p40 promoter, or a variant, e.g., functional fragment, thereof.
   E520. The variant baculovirus genome of any one of embodiments E517-E519, wherein the promoter is a ctx promoter.
   E521. The variant baculovirus genome of embodiment E519 or E520, wherein the ctx promoter comprises the promoter region of the ctx gene (e.g., AcORF3) and the 5′ UTR of the ctx gene.
   E522. The variant baculovirus genome of embodiment E519-E521, wherein the ctx promoter comprises the nucleotide sequence of any of SEQ ID NOs: 164-166; a nucleotide sequence at least 70%, 75%, 80%, 85%, 90%, 95%, 96%, 97%, 98%, or 99% identical to any of SEQ ID NOs: 164-166; a nucleotide sequence comprising at least one, two, three, four, five, six, or seven, but no more than ten different nucleotides relative to any of SEQ ID NOs: 164-166; or a nucleotide sequence comprising at least one, two, three, four, five, six, or seven, but no more than ten modifications (e.g., substitutions) relative to any of SEQ ID NOs: 164-166.
   E523. The variant baculovirus genome of embodiment E519-E522, wherein the ctx promoter comprises the nucleotide sequence of SEQ ID NO: 164; a nucleotide sequence at least 70%, 75%, 80%, 85%, 90%, 95%, 96%, 97%, 98%, or 99% identical to SEQ ID NOs: 164; a nucleotide sequence comprising at least one, two, three, four, five, six, or seven, but no more than ten different nucleotides relative to SEQ ID NO: 164; or a nucleotide sequence comprising at least one, two, three, four, five, six, or seven, but no more than ten modifications (e.g., substitutions) relative to SEQ ID NOs: 164.
   E524. The variant baculovirus genome of any one of embodiments E519-E523, which comprises in 5′ to 3′ order: a ctx promoter and the second VP-coding region comprising a nucleotide sequence encoding primarily a VP1 protein, e.g., at least 50%, 60%, 70%, 80%, 90% or more VP1 protein relative to a VP2 protein and or a VP3 protein (e.g., but not a VP2 protein or a VP3 protein).
   E525. The variant baculovirus genome of any one of embodiments E519-E524, which comprises in 5′ to 3′ order, a ctx promoter and the second VP-coding region comprising a nucleotide sequence encoding a VP1 protein only.
   E526. The variant baculovirus genome of any one of embodiments E519-E525, which comprises in 5′ to 3′ order, a ctx promoter and the second VP-coding region comprising a nucleotide sequence encoding a VP1 protein but not a VP2 protein or a VP3 protein.
   E527. The variant baculovirus genome of any one of embodiments E519-E526, wherein the second VP-coding region is present at a different location in the variant baculovirus genome than one, two, or all of the first Rep-coding region, the second Rep-coding region, or the VP-coding region.
   E528. The variant baculovirus genome of any one of embodiments E519-E527, wherein the second VP-coding region is present at a location in the variant baculovirus genome chosen from chiA, v-cath, p10, egt, polyhedrin, SOD, ctx, p26, odv-e56, p74 (PIF0), PIF1, PIF2, PIF3, PIF4, PIF5, Tn7, AcORF-91, AcORF-108, AcORF-52, v-ubi, or p94.
   E529. The variant baculovirus genome of any one of embodiments E519-E528, wherein the second VP-coding region is present in the SOD gene locus of the variant baculovirus genome.
   E530. The variant baculovirus genome of any one of embodiments E519-E529, wherein the second VP-coding region is present in the SOD gene locus of the variant baculovirus genome and is operably linked to a ctx promoter, optionally wherein the ctx promoter comprises the nucleotide sequence of SEQ ID NO: 164; a nucleotide sequence at least 70%, 75%, 80%, 85%, 90%, 95%, 96%, 97%, 98%, or 99% identical to SEQ ID NOs: 164; a nucleotide sequence comprising at least one, two, three, four, five, six, or seven, but no more than ten different nucleotides relative to SEQ ID NO: 164; or a nucleotide sequence comprising at least one, two, three, four, five, six, or seven, but no more than ten modifications (e.g., substitutions) relative to SEQ ID NOs: 164.
   E531. The variant baculovirus genome of any one of embodiments E519-E530, wherein the second VP-coding region is present in the SOD gene locus of the variant baculovirus genome and comprises a nucleotide sequence encoding primarily a VP1 protein, e.g., at least 50%, 60%, 70%, 80%, 90% or more VP1 protein relative to a VP2 protein or a VP3 protein (e.g., but not a VP2 or a VP3 protein).
   E532. The variant baculovirus genome of any one of embodiments E519-E531, wherein the second VP-coding region is present in the SOD gene locus of the variant baculovirus genome and wherein the second VP-coding region:
(i) comprises a nucleotide sequence encoding primarily a VP1 protein, e.g., at least 50%, 60%, 70%, 80%, 90% or more VP1 protein relative to a VP2 protein or a VP3 protein (e.g., but not a VP2 or a VP3 protein); and
(ii) is operably linked to a ctx promoter, optionally wherein the ctx promoter comprises the nucleotide sequence of SEQ ID NO: 164; a nucleotide sequence at least 70%, 75%, 80%, 85%, 90%, 95%, 96%, 97%, 98%, or 99% identical to SEQ ID NOs: 164; a nucleotide sequence comprising at least one, two, three, four, five, six, or seven, but no more than ten different nucleotides relative to SEQ ID NO: 164; or a nucleotide sequence comprising at least one, two, three, four, five, six, or seven, but no more than ten modifications (e.g., substitutions) relative to SEQ ID NOs: 164.
   E533. The variant baculovirus genome of any one of embodiments E519-E532, which comprises in 5′ to 3′ order, a ctx promoter; and the second VP-coding region comprising a nucleotide sequence encoding primarily a VP1 protein, e.g., at least 50%, 60%, 70%, 80%, 90% or more VP1 protein relative to a VP2 protein or a VP3 protein (e.g., but not a VP2 or a VP3 protein); wherein the second VP-coding region is present in the SOD gene locus of the variant baculovirus genome.
   E534. The variant baculovirus genome of any one of embodiments E519-E533, which comprises in 5′ to 3′ order, a ctx promoter; and a single ORF, comprising a start codon and a nucleotide sequence encoding primarily a VP1 protein, e.g., at least 50%, 60%, 70%, 80%, 90% or more VP1 protein relative to a VP2 protein or a VP3 protein (e.g., but not a VP2 or a VP3 protein); wherein the second VP-coding region is present in the SOD gene locus of the variant baculovirus genome.
   E535. The variant baculovirus genome of any one of embodiments E519-E534, which further comprises a modified Kozak sequence.
   E536. The variant baculovirus genome of embodiment E535, wherein the modified Kozak sequence is capable of modulating expression, e.g., increasing expression, of a protein encoded by a gene that is immediately downstream of the modified Kozak sequence.
   E537. The variant baculovirus genome of embodiment E535 or E536, wherein the modified Kozak sequence comprises a start codon for the translation of a protein encoded by a gene that is immediately downstream of the modified Kozak sequence.
   E538. The variant baculovirus genome of any one of embodiments E535-E537, wherein the modified Kozak comprises the nucleotide sequence of sequence of any one of SEQ ID NOs: 32-42, or a nucleotide sequence comprising no more than one, two, or three different nucleotides relative to SEQ ID NOs: 32-42.
   E539. The variant baculovirus genome of any one of embodiments E535-E538, wherein nucleotide sequence encoding the modified Kozak comprises the nucleotide sequence of any one of SEQ ID NOs: 21-31, 222, and 223, or a nucleotide sequence comprising no more than one, two, or three different nucleotides relative to SEQ ID NOs: 21-31, 222, and 223.
   E540. The variant baculovirus genome of any one of embodiments E535-E537, wherein the modified Kozak sequence comprises the nucleotide sequence of any one of SEQ ID NOs: 118-162, or a nucleotide sequence comprising no more than one, two, or three different nucleotides relative to SEQ ID NOs: 118-162.
   E541. The variant baculovirus genome of any one of embodiments E535-E537 or E540, wherein the nucleotide sequence encoding the modified Kozak sequence comprises the nucleotide sequence of any one of SEQ ID NOs: 73-117, or a nucleotide sequence comprising no more than one, two, or three different nucleotides relative to SEQ ID NOs: 73-117.
   E542. The variant baculovirus genome of any one of embodiments E535-E539, wherein the modified Kozak sequence comprises the nucleotide sequence of SEQ ID NO: 33, or a nucleotide sequence comprising no more than one, two, or three different nucleotides relative to SEQ ID NO: 33.
   E543. The variant baculovirus genome of any one of embodiments E535-E539 or E542, wherein nucleotide sequence encoding the modified Kozak sequence comprises the nucleotide sequence of SEQ ID NO: 22, or a nucleotide sequence comprising no more than one, two, or three different nucleotides relative to SEQ ID NO: 22.
   E544. The variant baculovirus genome of any one of embodiments E535-E539, wherein the modified Kozak sequence comprises the nucleotide sequence of SEQ ID NO: 32, or a nucleotide sequence comprising no more than one, two, or three different nucleotides relative to SEQ ID NO: 32.
   E545. The variant baculovirus genome of any one of embodiments E535-E539 or E544, wherein nucleotide sequence encoding the modified Kozak sequence comprises the nucleotide sequence of SEQ ID NO: 21, or a nucleotide sequence comprising no more than one, two, or three different nucleotides relative to SEQ ID NO: 21.
   E546. The variant baculovirus genome of any one of embodiments E535-E545, wherein the modified Kozak sequence is present at the 5′ end of the VP-coding region, e.g., at the start of the VP-coding region encoding the VP1 protein (e.g., the ORF encoding the VP1 protein).
   E547. The variant baculovirus genome of any one of embodiments E535-E546, wherein the modified Kozak sequence comprises the start codon of the ORF encoding the VP1 protein.
   E548. The variant baculovirus genome of any one of embodiments E535-E547, wherein the start codon comprises an ATG.
   E549. The variant baculovirus genome of any one of embodiments E535-E539 or E542-E548, wherein the modified Kozak sequence and the VP-coding region comprises the nucleotide sequence of SEQ ID NO:44 or 45, or a nucleotide sequence at least 70%, 75%, 80%, 85%, 90%, 95%, 96%, 97%, 98%, or 99% identical to the aforesaid sequences.
   E550. The variant baculovirus genome of any one of embodiments E535-E539, E542, E543, or E546-E549, wherein the VP-coding region comprising a modified Kozak sequence encodes a VP1 protein comprising the amino acid sequence of SEQ ID NOs:46, 47, 71, or 168, or an amino acid sequence at least 70%, 75%, 80%, 85%, 90%, 95%, 96%, 97%, 98%, or 99% identical to the aforesaid sequences.
   E551. The variant baculovirus genome of any one of embodiments E535-E539 or E544-E548, wherein the VP-coding region comprising a modified Kozak sequence encodes a VP1 protein comprising the amino acid sequence of SEQ ID NOs:46, 47, 71, or 168, or an amino acid sequence at least 70%, 75%, 80%, 85%, 90%, 95%, 96%, 97%, 98%, or 99% identical to the aforesaid sequences.
   E552. The variant baculovirus genome of any one of embodiments E535-E551, which comprises in 5′ to 3′ order, a p10 promoter, and a VP-coding region comprising a modified Kozak sequence and a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein;
wherein the modified Kozak sequence is present at the 5′ end of the VP-coding region, e.g., at the start of the VP-coding region encoding the VP1 protein (e.g., the ORF encoding the VP1 protein);
optionally wherein, the modified Kozak sequence comprises the nucleotide sequence of SEQ ID NO: 32 or SEQ ID NO: 33, or a nucleotide sequence comprising no more than one, two, or three different nucleotides relative to SEQ ID NO: 32 or SEQ ID NO: 33.
   E553. The variant baculovirus genome of any one of embodiments E362-E365, E374-E381, E386-E390, E412-418, E423-E433, E437, E440-E444, or E456-E552, wherein:
(i) the first Rep-coding region is present in the v-cath locus of the variant baculovirus genome; (ii) the second Rep-coding region is present in the egt locus of the variant baculovirus genome; and
(iii) the VP-coding region is present in the v-cath locus of the variant baculovirus genome.
   E554. The variant baculovirus genome of any one of embodiments E359, E360, E366-E373, E383-E385, E391-E411, E419-E422, E434-E436, E445-E475, E480, E483, E485, E487, E489, E491, E493, E495, E497, E499, or E501-E552, wherein:
(i) the Rep-coding region is present in the p74 locus of the variant baculovirus genome; and
(ii) the VP-coding region is present in the SOD locus of the variant baculovirus genome.
   E555. The variant baculovirus genome of any one of embodiments E362-E365, E374-E381, E386-E390, E412-418, E423-E433, E437, E440-E444, or E456-E553, wherein:
(i) the first Rep-coding region is present in the v-cath locus of the variant baculovirus genome and comprises a nucleotide sequence encoding primarily a Rep78 protein, e.g., at least 50%, 60%, 70%, 80%, 90% or more Rep78 protein relative to a Rep52 protein (e.g., but not a Rep52 protein);
(ii) the second Rep-coding region is present in the egt locus of the variant baculovirus genome and comprises a nucleotide sequence encoding a Rep52 protein but not a Rep78 protein; and
(iii) the VP-coding region is present in the v-cath locus of the variant baculovirus genome and comprises a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein.
   E556. The variant baculovirus genome of any one of embodiments E359, E360, E366-E373, E383-E385, E391-E411, E419-E422, E434-E436, E445-E475, E480, E483, E485, E487, E489, E491, E493, E495, E497, E499, E501-E552, or E554, wherein:
(i) the Rep-coding region is present in the p74 locus of the variant baculovirus genome and comprises a nucleotide sequence encoding a Rep78 protein and a Rep52 protein, wherein the nucleotide sequence encoding the Rep52 protein are comprised within the nucleotide sequence encoding the Rep78 protein; and
(ii) the VP-coding region is present in the SOD locus of the variant baculovirus genome and comprises a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein.
   E557. The variant baculovirus genome of any one of embodiments E362-E365, E374-E381, E386-E390, E412-418, E423-E433, E437, E440-E444, E456-E553, or E555, wherein:
(i) the first Rep-coding region is present in the v-cath locus of the variant baculovirus genome and is operably linked to a polh promoter;
(ii) the second Rep-coding region is present in the egt locus of the variant baculovirus genome and is operably linked to a polh promoter; and
(iii) the VP-coding region is present in the v-cath locus of the variant baculovirus genome and is operably linked to a p10 promoter.
   E558. The variant baculovirus genome of any one of embodiments E359, E360, E366-E373, E383-E385, E391-E411, E419-E422, E434-E436, E445-E475, E480, E483, E485, E487, E489, E491, E493, E495, E497, E499, E501-E552, E554, or E556, wherein:
(i) the Rep-coding region is present in the p74 gene locus of the variant baculovirus genome and is operably linked to a gp64 promoter and polh promoter; and
(iii) the VP-coding region is present in the SOD locus of the variant baculovirus genome and is operably linked to a p10 promoter.
   E559. The variant baculovirus genome of any one of embodiments E362-E365, E374-E381, E386-E390, E412-418, E423-E433, E437, E440-E444, E456-E553, E555, or E557, wherein:
(i) the first Rep-coding region is present in the v-cath locus of the variant baculovirus genome and comprises a nucleotide sequence encoding primarily a Rep78 protein, e.g., at least 50%, 60%, 70%, 80%, 90% or more Rep78 protein relative to a Rep52 protein (e.g., but not a Rep52 protein), wherein the first Rep-coding region is operably linked to a polh promoter;
(ii) the second Rep-coding region is present in the egt locus of the variant baculovirus genome and comprises a nucleotide sequence encoding a Rep52 protein but not a Rep78 protein, wherein the second Rep-coding region is operably linked to a polh promoter; and
(iii) the VP-coding region is present in the v-cath locus of the variant baculovirus genome and comprises a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein, and wherein the VP-coding region is operably linked to a p10 promoter;
optionally wherein the VP-coding region is present in the reverse orientation relative to the first Rep-coding region.
   E560. The variant baculovirus genome of any one of embodiments E359, E360, E366-E373, E383-E385, E391-E411, E419-E422, E434-E436, E445-E475, E480, E483, E485, E487, E489, E491, E493, E495, E497, E499, E501-E552, E554, E556, or E558, wherein:
(i) the Rep-coding region is present in the p74 gene locus of the variant baculovirus genome and comprises a nucleotide sequence encoding a Rep78 protein and a Rep52 protein, wherein the nucleotide sequence encoding the Rep52 protein is comprised within the nucleotide sequence encoding the Rep78 protein, wherein the first Rep-coding region is operably linked to a polh promoter; and
(ii) the VP-coding region is present in the SOD locus of the variant baculovirus genome and comprises a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein, and wherein the VP-coding region is operably linked to a p10 promoter;
optionally wherein the VP-coding region is present in the reverse orientation relative to the Rep-coding region.
   E561. The variant baculovirus genome of any one of embodiments E362-E365, E374-E381, E386-E390, E412-418, E423-E433, E437, E440-E444, E456-E553, E555, E557, or E559, wherein:
(i) the first Rep-coding region is present in the v-cath locus of the variant baculovirus genome;
(ii) the second Rep-coding region is present in the egt locus of the variant baculovirus genome;
(iii) the VP-coding region is present in the v-cath locus of the variant baculovirus genome; and
(iv) the second VP-coding region is present in the SOD locus of the variant baculovirus genome.
   E562. The variant baculovirus genome of any one of embodiments E362-E365, E374-E381, E386-E390, E412-418, E423-E433, E437, E440-E444, E456-E553, E555, E557, E559, or E561, wherein:
(i) the first Rep-coding region is present in the v-cath locus of the variant baculovirus genome and comprises a nucleotide sequence encoding primarily a Rep78 protein, e.g., at least 50%, 60%, 70%, 80%, 90% or more Rep78 protein relative to a Rep52 protein (e.g., but not a Rep52 protein);
(ii) the second Rep-coding region is present in the egt locus of the variant baculovirus genome and comprises a nucleotide sequence encoding a Rep52 protein but not a Rep78 protein;
(iii) the VP-coding region is present in the v-cath locus of the variant baculovirus genome and comprises a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein; and
(iv) the second VP-coding region is present in the SOD locus of the variant baculovirus genome and comprises a nucleotide sequence encoding primarily a VP1 protein, e.g., at least 50%, 60%, 70%, 80%, 90% or more VP1 protein relative to a VP2 protein or a VP3 protein (e.g., but not a VP2 or a VP3 protein).
   E563. The variant baculovirus genome of any one of embodiments E362-E365, E374-E381, E386-E390, E412-418, E423-E433, E437, E440-E444, E456-E553, E555, E557, E559, E561, or E562, wherein:
(i) the first Rep-coding region is present in the v-cath locus of the variant baculovirus genome and is operably linked to a polh promoter;
(ii) the second Rep-coding region is present in the egt locus of the variant baculovirus genome and is operably linked to a polh promoter;
(iii) the VP-coding region is present in the v-cath locus of the variant baculovirus genome and is operably linked to a p10 promoter; and
(iv) the second VP-coding region is present in the SOD locus of the variant and is operably linked to a ctx promoter.
   E564. The variant baculovirus genome of any one of embodiments E362-E365, E374-E381, E386-E390, E412-418, E423-E433, E437, E440-E444, E456-E553, E555, E557, E559, or E561-E563, wherein:
(i) the first Rep-coding region is present in the v-cath locus of the variant baculovirus genome and comprises a nucleotide sequence encoding primarily a Rep78 protein, e.g., at least 50%, 60%, 70%, 80%, 90% or more Rep78 protein relative to a Rep52 protein (e.g., but not a Rep52 protein), and wherein the first Rep-coding region is operably linked to a polh promoter;
(ii) the second Rep-coding region is present in the egt locus of the variant baculovirus genome and comprises a nucleotide sequence encoding a Rep52 protein but not a Rep78 protein, and wherein the second Rep-coding region is operably linked to a polh promoter;
(iii) the VP-coding region is present in the v-cath locus of the variant baculovirus genome and comprises a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein, and wherein the VP-coding region is operably linked to a p10 promoter; and
(iv) the second VP-coding region is present in the SOD locus of the variant baculovirus genome, and comprises a nucleotide sequence encoding primarily a VP1 protein, e.g., at least 50%, 60%, 70%, 80%, 90% or more VP1 protein relative to a VP2 protein or a VP3 protein (e.g., but not a VP2 or a VP3 protein), and wherein the second VP-coding region is operably linked to a ctx promoter;
optionally wherein, the VP-coding region is present in the reverse orientation relative to the first Rep-coding region.
   E565. The variant baculovirus genome of any one of embodiments E362-E365, E374-E381, E386-E390, E412-418, E423-E433, E437, E440-E444, E456-E553, E555, E557, or E559, wherein:
(i) the first Rep-coding region is present in the v-cath locus of the variant baculovirus genome and comprises a nucleotide sequence encoding primarily a Rep78 protein, e.g., at least 50%, 60%, 70%, 80%, 90% or more Rep78 protein relative to a Rep52 protein (e.g., but not a Rep52 protein);
(ii) the second Rep-coding region is present in the egt locus of the variant baculovirus genome and comprises a nucleotide sequence encoding a Rep52 protein but not a Rep78 protein; and
(iii) the VP-coding region is present in the v-cath locus of the variant baculovirus genome, wherein the VP-coding region comprises a modified Kozak sequence, optionally wherein the modified Kozak sequence comprises the nucleotide sequence of SEQ ID NO: 32 or SEQ ID NO: 33, or a nucleotide sequence comprising no more than one, two, or three different nucleotides relative to SEQ ID NO: 32 or SEQ ID NO: 33.
   E566. The variant baculovirus genome of any one of embodiments E359, E360, E366-E373, E383-E385, E391-E411, E419-E422, E434-E436, E445-E475, E480, E483, E485, E487, E489, E491, E493, E495, E497, E499, E501-E552, E554, E556, E558, or E560, wherein:
(i) the Rep-coding region is present in the p74 gene locus of the variant baculovirus genome and comprises a nucleotide sequence encoding a Rep78 protein and Rep52 protein, wherein the nucleotide sequence encoding the Rep52 protein is comprised within the nucleotide sequence encoding the Rep78 protein; and
(ii) the VP-coding region is present in the SOD locus of the variant baculovirus genome, wherein the VP-coding region comprises a modified Kozak sequence, optionally wherein the modified Kozak sequence comprises the nucleotide sequence of SEQ ID NO: 32 or SEQ ID NO: 33, or a nucleotide sequence comprising no more than one, two, or three different nucleotides relative to SEQ ID NO: 32 or SEQ ID NO: 33.
   E567. The variant baculovirus genome of any one of embodiments E362-E365, E374-E381, E386-E390, E412-418, E423-E433, E437, E440-E444, E456-E553, E555, E557, E559, or E565, wherein:
(i) the first Rep-coding region is present in the v-cath locus of the variant baculovirus genome and comprises a nucleotide sequence encoding primarily a Rep78 protein, e.g., at least 50%, 60%, 70%, 80%, 90% or more Rep78 protein relative to a Rep52 protein (e.g., but not a Rep52 protein);
(ii) the second Rep-coding region is present in the egt locus of the variant baculovirus genome and comprises a nucleotide sequence encoding a Rep52 protein but not a Rep78 protein; and
(iii) the VP-coding region is present in the v-cath locus of the variant baculovirus genome, wherein the VP-coding region comprises a modified Kozak sequence, which is present at the 5′ end of the VP-coding region, e.g., at the start of the VP-coding region encoding the VP1 protein (e.g., the ORF encoding the VP1 protein), optionally wherein the modified Kozak sequence comprises the nucleotide sequence of SEQ ID NO: 32 or SEQ ID NO: 33, or a nucleotide sequence comprising no more than one, two, or three different nucleotides relative to SEQ ID NO: 32 or SEQ ID NO: 33.
   E568. The variant baculovirus genome of any one of embodiments E359, E360, E366-E373, E383-E385, E391-E411, E419-E422, E434-E436, E445-E475, E480, E483, E485, E487, E489, E491, E493, E495, E497, E499, E501-E552, E554, E556, E558, E560, or E566, wherein:
(i) the Rep-coding region is present in the p74 gene locus of the variant baculovirus genome and comprises a nucleotide sequence encoding a Rep78 protein and Rep52 protein; and
(ii) the VP-coding region is present in the SOD locus of the variant baculovirus genome, wherein the VP-coding region comprises a modified Kozak sequence, which is present at the 5′ end of the VP-coding region, e.g., at the start of the VP-coding region encoding the VP1 protein (e.g., the ORF encoding the VP1 protein), optionally wherein the modified Kozak sequence comprises the nucleotide sequence of SEQ ID NO: 32 or SEQ ID NO: 33, or a nucleotide sequence comprising no more than one, two, or three different nucleotides relative to SEQ ID NO: 32 or SEQ ID NO: 33.
   E569. The variant baculovirus genome of any one of embodiments E362-E365, E374-E381, E386-E390, E412-418, E423-E433, E437, E440-E444, E456-E553, E555, E557, E559, E565, or E567, wherein:
(i) the first Rep-coding region is present in the v-cath locus of the variant baculovirus genome and comprises a nucleotide sequence encoding primarily a Rep78 protein, e.g., at least 50%, 60%, 70%, 80%, 90% or more Rep78 protein relative to a Rep52 protein (e.g., but not a Rep52 protein), and wherein the first Rep-coding region is operably linked to a polh promoter;
(ii) the second Rep-coding region is present in the egt locus of the variant baculovirus genome and comprises a nucleotide sequence encoding a Rep52 protein but not a Rep78 protein, and wherein the second Rep-coding region is operably linked to a polh promoter;
(iii) the VP-coding region is present in the v-cath locus of the variant baculovirus genome and is operably linked to a p10 promoter, wherein the VP region comprises:
   (a) a modified Kozak sequence (e.g., comprising the nucleotide sequence of SEQ ID NO: 32 or SEQ ID NO: 33, or a nucleotide sequence comprising no more than one, two, or three different nucleotides relative to SEQ ID NO: 32 or SEQ ID NO: 33), which is present at the 5′ end of the VP-coding region (e.g., at the start of the VP-coding region); and
   (b) a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein.
      E570. The variant baculovirus genome of any one of embodiments E359, E360, E366-E373, E383-E385, E391-E411, E419-E422, E434-E436, E445-E475, E480, E483, E485, E487, E489, E491, E493, E495, E497, E499, E501-E552, E554, E556, E558, E560, E566, or E568, wherein:
(i) the Rep-coding region is present in the p74 gene locus of the variant baculovirus genome and comprises a nucleotide sequence encoding a Rep78 protein and Rep52 protein, and wherein the first Rep-coding region is operably linked to a gp64 promoter and a polh promoter; and
(ii) the VP-coding region is present in the SOD locus of the variant baculovirus genome and is operably linked to a p10 promoter, wherein the VP-coding region comprises:
   (a) a modified Kozak sequence (e.g., comprising the nucleotide sequence of SEQ ID NO: 32 or SEQ ID NO: 33, or a nucleotide sequence comprising no more than one, two, or three different nucleotides relative to SEQ ID NO: 32 or SEQ ID NO: 33), which is present at the 5′ end of the VP-coding region (e.g., at the start of the VP-coding region); and
   (b) a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein.
      E571. The variant baculovirus genome of any one of embodiments E362-E365, E374-E381, E386-E390, E412-418, E423-E433, E437, E440-E444, E456-E553, E555, E557, E559, E565, E567, or E569, wherein:
(i) the first Rep-coding region is present in the v-cath locus of the variant baculovirus genome and comprises a nucleotide sequence encoding primarily a Rep78 protein, e.g., at least 50%, 60%, 70%, 80%, 90% or more Rep78 protein relative to a Rep52 protein (e.g., but not a Rep52 protein), and wherein the first Rep-coding region is operably linked to a polh promoter;
(ii) the second Rep-coding region is present in the egt locus of the variant baculovirus genome and comprises a nucleotide sequence encoding a Rep52 protein but not a Rep78 protein, and wherein the second Rep-coding region is operably linked to a polh promoter;
(iii) the VP-coding region is present in the v-cath locus of the variant baculovirus genome and is operably linked to a p10 promoter, wherein the VP region comprises in 5′ to 3′ order:
(a) a modified Kozak sequence, optionally comprising the nucleotide sequence of SEQ ID NO: 32 or SEQ ID NO: 33, or a nucleotide sequence comprising no more than one, two, or three different nucleotides relative to SEQ ID NO: 32 or SEQ ID NO: 33; and
   (b) a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein.
      E572. The variant baculovirus genome of any one of embodiments E359, E360, E366-E373, E383-E385, E391-E411, E419-E422, E434-E436, E445-E475, E480, E483, E485, E487, E489, E491, E493, E495, E497, E499, E501-E552, E554, E556, E558, E560, E566, E568, or E570, wherein:
(i) the Rep-coding region is present in the p74 gene locus of the variant baculovirus genome and comprises a nucleotide sequence encoding a Rep78 protein and Rep52 protein, wherein the nucleotide sequence encoding the Rep52 protein is comprised within the nucleotide sequence encoding the Rep78 protein, and wherein the first Rep-coding region is operably linked to a gp64 promoter and a polh promoter; and
(ii) the VP-coding region is present in the SOD locus of the variant baculovirus genome and is operably linked to a p10 promoter, wherein the VP region comprises in 5′ to 3′ order:
   (a) a modified Kozak sequence, optionally comprising the nucleotide sequence of SEQ ID NO: 32 or SEQ ID NO: 33, or a nucleotide sequence comprising no more than one, two, or three different nucleotides relative to SEQ ID NO: 32 or SEQ ID NO: 33; and
   (b) a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein.
      E573. A variant baculovirus genome comprising an AAV expression construct comprising:
(i) a first Rep-coding region which is present in the v-cath locus of the variant baculovirus genome;
(ii) a second Rep-coding which region is present in the egt locus of the variant baculovirus genome; and
(iii) a VP-coding region which is present in the v-cath locus of the variant baculovirus genome.
   E574. A variant baculovirus genome comprising an AAV expression construct comprising:
(i) a first Rep-coding region which is present in the v-cath locus of the variant baculovirus genome and is operably linked to a polh promoter;
(ii) a second Rep-coding region which is present in the egt locus of the variant baculovirus genome and is operably linked to a polh promoter; and
(iii) a VP-coding region which is present in the v-cath locus of the variant baculovirus genome and is operably linked to a p10 promoter.
   E575. A variant baculovirus genome comprising an AAV expression construct comprising:
(i) a first Rep-coding region, which is present in the v-cath locus of the variant baculovirus genome and comprises a nucleotide sequence encoding primarily a Rep78 protein, e.g., at least 50%, 60%, 70%, 80%, 90% or more Rep78 protein relative to a Rep52 protein (e.g., but not a Rep52 protein);
(ii) a second Rep-coding region, which is present in the egt locus of the variant baculovirus genome and comprises a nucleotide sequence encoding a Rep52 protein but not a Rep78 protein; and
(iii) a VP-coding region, which is present in the v-cath locus of the variant baculovirus genome and comprises a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein.
   E576. A variant baculovirus genome comprising an AAV expression construct comprising:
(i) a first Rep-coding region, which is present in the v-cath locus of the variant baculovirus genome and comprises a nucleotide sequence encoding primarily a Rep78 protein, e.g., at least 50%, 60%, 70%, 80%, 90% or more Rep78 protein relative to a Rep52 protein (e.g., but not a Rep52 protein), wherein the first Rep-coding region is operably linked to a polh promoter;
(ii) a second Rep-coding region is present in the egt locus of the variant baculovirus genome and comprises a nucleotide sequence encoding a Rep52 protein but not a Rep78 protein, wherein the second Rep-coding region is operably linked to a polh promoter; and
(iii) a VP-coding region which is present in the v-cath locus of the variant baculovirus genome and comprises a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein, and wherein the VP-coding region is operably linked to a p10 promoter;
optionally wherein the VP-coding region is present in the reverse orientation relative to the first Rep-coding region.
   E577. A variant baculovirus genome comprising an AAV expression construct comprising:
(i) a first Rep-coding region, which is present in the v-cath locus of the variant baculovirus genome;
(ii) a second Rep-coding region, which is present in the egt locus of the variant baculovirus genome;
(iii) a VP-coding region, which is present in the v-cath locus of the variant baculovirus genome; and
(iv) a second VP-coding region, which is present in the SOD locus of the variant baculovirus genome.
   E578. A variant baculovirus genome comprising an AAV expression construct comprising:
(i) a first Rep-coding region, which is present in the v-cath locus of the variant baculovirus genome and comprises a nucleotide sequence encoding primarily a Rep78 protein, e.g., at least 50%, 60%, 70%, 80%, 90% or more Rep78 protein relative to a Rep52 protein (e.g., but not a Rep52 protein);
(ii) a second Rep-coding region, which is present in the egt locus of the variant baculovirus genome and comprises a nucleotide sequence encoding a Rep52 protein but not a Rep78 protein;
(iii) a VP-coding region, which is present in the v-cath locus of the variant baculovirus genome and comprises a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein; and
(iv) a second VP-coding region, which is present in the SOD locus of the variant baculovirus genome and comprises a nucleotide sequence encoding primarily a VP1 protein, e.g., at least 50%, 60%, 70%, 80%, 90% or more VP1 protein relative to a VP2 protein or a VP3 protein (e.g., but not a VP2 or a VP3 protein).
   E579. A variant baculovirus genome comprising an AAV expression construct comprising:
(i) a first Rep-coding region, which is present in the v-cath locus of the variant baculovirus genome and comprises a nucleotide sequence encoding primarily a Rep78 protein, e.g., at least 50%, 60%, 70%, 80%, 90% or more Rep78 protein relative to a Rep52 protein (e.g., but not a Rep52 protein), wherein the first Rep-coding region is operably linked to a polh promoter;
(ii) a second Rep-coding region, which is present in the egt locus of the variant baculovirus genome and comprises a nucleotide sequence encoding a Rep52 protein but not a Rep78 protein, and wherein the second Rep-coding region is operably linked to a polh promoter;
(iii) a VP-coding region, which is present in the v-cath locus of the variant baculovirus genome and comprises a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein, and wherein the VP-coding region is operably linked to a p10 promoter; and
(iv) a second VP-coding region, which is present in the SOD locus of the variant baculovirus genome, and comprises a nucleotide sequence encoding primarily a VP1 protein, e.g., at least 50%, 60%, 70%, 80%, 90% or more VP1 protein relative to a VP2 protein or a VP3 protein (e.g., but not a VP2 or a VP3 protein), wherein the second VP-coding region is operably linked to a ctx promoter;
optionally wherein, the VP-coding region is present in the reverse orientation relative to the first Rep-coding region.
   E580. A variant baculovirus genome comprising an AAV expression construct comprising:
(i) a first Rep-coding region, which is present in the v-cath locus of the variant baculovirus genome and comprises a nucleotide sequence encoding primarily a Rep78 protein, e.g., at least 50%, 60%, 70%, 80%, 90% or more Rep78 protein relative to a Rep52 protein (e.g., but not a Rep52 protein), and wherein the first Rep-coding region is operably linked to a polh promoter;
(ii) a second Rep-coding region, which is present in the egt locus of the variant baculovirus genome and comprises a nucleotide sequence encoding a Rep52 protein but not a Rep78 protein, and wherein the second Rep-coding region is operably linked to a polh promoter; and
(iii) a VP-coding region, which is present in the v-cath locus of the variant baculovirus genome and is operably linked to a p10 promoter, wherein the VP region comprises:
   (a) a modified Kozak sequence which is present at the 5′ end of the VP-coding region (e.g., at the start of the VP-coding region), optionally wherein the modified Kozak sequence comprises the nucleotide sequence of SEQ ID NO: 32 or SEQ ID NO: 33, or a nucleotide sequence comprising no more than one, two, or three different nucleotides relative to SEQ ID NO: 32 or SEQ ID NO: 33; and
   (b) a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein.
      E581. The variant baculovirus genome of any one of embodiments E196-E471, further comprising a nucleotide sequence encoding an assembly-activating protein (AAP).
      E582. The variant baculovirus genome of embodiment E581, wherein the encoded AAP protein is an AAV2 AAP protein.
      E583. The variant baculovirus genome of embodiment E581 or E582, wherein the encoded AAP protein comprises the amino acid sequence of SEQ ID NO: 218; an amino acid sequence comprising at least 70%, 75%, 80%, 85%, 90%, 95%, 96%, 97%, 98%, 99% sequence identity to SEQ ID NO: 218; an amino acid sequence comprising at least one, two, three, four, five, six or seven modifications, e.g., substitutions (e.g., conservative substitutions), insertions, or deletions, but no more than 30, 20, or 10 modifications, e.g., substitutions (e.g., conservative substitutions), insertions, or deletions, relative to SEQ ID NO: 218; or an amino acid sequence comprising at least one, two, three, four, five, six or seven, but no more than 30, 20, or 10 different amino acids relative to SEQ ID NO: 218.
      E584. The variant baculovirus genome of any one of embodiments E581-E583, wherein the nucleotide sequence encoding the AAP protein comprises the nucleotide sequence of SEQ ID NO: 219; a nucleotide sequence comprising at least 70%, 75%, 80%, 85%, 90%, 95%, 96%, 97%, 98%, 99% sequence identity to SEQ ID NO: 219; a nucleotide sequence comprising at least one, two, three, four, five, six or seven modifications, e.g., substitutions (e.g., conservative substitutions), insertions, or deletions, but no more than 30, 20, or 10 modifications, e.g., substitutions (e.g., conservative substitutions), insertions, or deletions, relative to SEQ ID NO: 219; or a nucleotide sequence comprising at least one, two, three, four, five, six or seven, but no more than 30, 20, or 10 different nucleotides relative to SEQ ID NO: 219.
      E585. The variant baculovirus genome of any one of embodiments E581-E584, wherein the nucleotide sequence encoding the AAP protein is operably linked to a promoter.
      E586. The variant baculovirus genome of embodiment E585, wherein the promoter is a baculovirus major late promoter, a viral promoter, an insect viral promoter, a non-insect viral promoter, a vertebrate viral promoter, a chimeric promoter from one or more species including virus and non-virus elements, a synthetic promoter, or a variant thereof.
      E587. The variant baculovirus genome of embodiment E585 or E586, wherein the promoter is chosen from a polh promoter, a p10 promoter, a ctx promoter, a gp64 promoter an IE promoter, an IE-1 promoter, a p6.9 promoter, a Dmhsp70 promoter, a Hsp70 promoter, a p5 promoter, a p19 promoter, a p35 promoter, a p40 promoter, or a variant, e.g., functional fragment, thereof.
      E588. The variant baculovirus genome of any one of embodiments E585-E587, wherein the promoter is a gp64 promoter, optionally wherein the promoter is gp64 promoter from a OpMNPV baculovirus genome.
      E589. The variant baculovirus genome of embodiment E587 or E588, wherein the gp64 promoter comprises the nucleotide sequence of SEQ ID NO: 217; a nucleotide sequence at least 70%, 75%, 80%, 85%, 90%, 95%, 96%, 97%, 98%, or 99% identical thereto; a nucleotide sequence comprising at least one, two, three, four, five, six, or seven, but no more than ten different nucleotides relative to SEQ ID NO: 217; or a nucleotide sequence comprising at least one, two, three, four, five, six, or seven, but no more than ten modifications (e.g., substitutions) relative to SEQ ID NO: 217.
      E590. The variant baculovirus genome of any one of embodiments E581-E589, wherein the nucleotide sequence encoding the AAP protein is present in a location in the variant baculovirus genome chosen from ChiA, v-cath, p10, egt, polyhedrin, SOD, ctx, p26, odv-e56, p74 (PIF0), PIF1, PIF2, PIF3, PIF4, PIF5, Tn7, AcORF-91, AcORF-108, AcORF-52, v-ubi, or p94.
      E591. The variant baculovirus genome of any one of embodiments E581-E590, wherein the nucleotide sequence encoding the AAP protein is present in the p26, p10, and p74 gene loci.
      E592. The variant baculovirus genome of any one of embodiments E581-E591, wherein the nucleotide sequence encoding the AAP protein is present in the p26, p10, and p74 gene loci, and is operably linked to a gp64 promoter.
      E593. The variant baculovirus genome of any one of embodiments E581-E592, which further comprises a payload coding region comprising a nucleotide sequence encoding a payload.
      E594. A variant baculovirus genome comprising an AAV expression construct comprising:
(i) a Rep-coding region which is present in the p74 locus of the variant baculovirus genome, optionally wherein the Rep-coding region is operably linked to a gp64 promoter and a polh promoter;
(ii) a VP-coding region which is present in the SOD locus of the variant baculovirus genome, optionally wherein the VP-coding region is operably linked to a p10 promoter; and
(iii) a payload coding region which is present in the v-cath locus of the variant baculovirus genome;
optionally wherein the VP-coding region is present in the reverse orientation relative to the Rep-coding region.
   E595. A variant baculovirus genome comprising an AAV expression construct comprising:
(i) a Rep-coding region, which is present in the p74 locus of the variant baculovirus genome and comprises a nucleotide sequence encoding a Rep78 protein and a Rep52 protein, wherein the nucleotide sequence encoding the Rep52 protein is comprised within the nucleotide sequence encoding the Rep78 protein, optionally wherein the Rep-coding region is operably linked to a gp64 promoter and a polh promoter;
(ii) a VP-coding region, which is present in the SOD locus of the variant baculovirus genome and comprises a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein; optionally wherein the VP-coding region is operably linked to a p10 promoter; and
(iii) a payload coding region which is present in the v-cath locus of the variant baculovirus genome and comprises a nucleotide sequence encoding a payload;
optionally wherein the VP-coding region is present in the reverse orientation relative to the Rep-coding region.
   E596. A variant baculovirus genome comprising an AAV expression construct comprising:
(i) a Rep-coding region, which is present in the p74 locus of the variant baculovirus genome and comprises a nucleotide sequence encoding a Rep78 protein and a Rep52 protein, wherein the nucleotide sequence encoding the Rep52 protein is comprised within the nucleotide sequence encoding the Rep78 protein, optionally wherein the Rep-coding region is operably linked to a gp64 promoter and a polh promoter;
(ii) a VP-coding region, which is present in the SOD locus of the variant baculovirus genome and is operably linked to a p10 promoter, wherein the VP region comprises:
   (a) a modified Kozak sequence which is present at the 5′ end of the VP-coding region (e.g., at the start of the VP-coding region), optionally wherein the modified Kozak sequence comprises the nucleotide sequence of SEQ ID NO: 32 or SEQ ID NO: 33, or a nucleotide sequence comprising no more than one, two, or three different nucleotides relative to SEQ ID NO: 32 or SEQ ID NO: 33; and
   (b) a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein.
(iii) a payload coding region which is present in the v-cath locus of the variant baculovirus genome, and comprises a nucleotide sequence encoding a payload;
   optionally wherein the VP-coding region is present in the reverse orientation relative to the Rep-coding region.
   E597. The variant baculovirus genome of any one of embodiments E292-E596, which further comprises an AAV payload construct.
   E598. A variant baculovirus genome comprising an AAV payload construct comprising a payload coding region, wherein the variant baculovirus genome comprises a disruption of at least two non-essential genes (e.g., auxiliary and/or per os infectivity factor genes), wherein the at least two non-essential genes are independently chosen from egt, p74 (PIF0), p26, SOD, ChiA, v-cath, p10, polyhedrin, ctx, odv-e56, PIF1, PIF2, PIF3, PIF4, PIF5, Tn7, AcORF-91, AcORF-108, AcORF-52, v-ubi, or p94.
   E599. The variant baculovirus genome of embodiment E597 or E598, wherein the payload coding region is present in a location in the variant baculovirus genome chosen from ChiA, v-cath, p10, egt, polyhedrin, SOD, ctx, p26, odv-e56, p74 (PIF0), PIF1, PIF2, PIF3, PIF4, PIF5, Tn7, AcORF-91, AcORF-108, AcORF-52, v-ubi, or p94.
   E600. The variant baculovirus genome of any one of embodiments E593-E599, wherein the payload coding region comprises a start codon and a nucleotide sequence encoding the payload.
   E601. The variant baculovirus genome of any one of embodiments E593-E600, wherein the payload coding region is present in the v-cath locus of the variant baculovirus genome.
   E602. The variant baculovirus genome of any one of embodiments E593-E601, or the AAV payload construct of any one of embodiments E434-E438, wherein the encoded payload comprises a therapeutic protein or functional variant thereof; an antibody or antibody fragment; an enzyme; a component of a gene editing system; an RNAi agent (e.g., a dsRNA, siRNA, shRNA, pre-miRNA, pri-miRNA, miRNA, stRNA, lncRNA, piRNA, or snoRNA); or a combination thereof.
   E603. A variant baculovirus genome which comprises the AAV expression construct according to any one of embodiments E292-E602, and the AAV payload construct of any one of embodiments E597-E602, wherein the AAV expression construct and the AAV payload construct are present in a single variant baculovirus genome.
   E604. The variant baculovirus genome of any one of embodiments E292-E603, which the genome sequence is generated (e.g., designed) in silico.
   E605. The variant baculovirus genome of any one of embodiments E292-E604, which is capable of producing at least 70% (e.g., at least 80%, at least 90%, at least 100%, at least 110%, at least 120%, 70-200%, 70-150%, 70-100%, 80-200%, 80-150%, 80-100%, 90-200%, 90-150%, 90-100%, 100-200%, 100-150%, 100-125%, 100-110%, 125-200%, 150-200%, or 175-200%) of the baculovirus produced by a reference baculovirus genome (e.g., a wild-type baculovirus genome).
   E606. The variant baculovirus genome of any one of embodiments E292-E605, wherein the variant baculovirus genome is at least 10 kb-200 kb in length, e.g., at least 10 kb, 20 kb, 30 kb, 40 kb, 50 kb, 60 kb, 70 kb, 80 kb, 90 kb, 100 kb, 110 kb, 120 kb, 130 kb, or 140 kb, or 10-175 kb, 10-150 kb, 10-125 kb, 10-100 kb, 10-75 kb, 10-50 kb, 10-25 kb, 25-200 kb, 25-175 kb, 25-150 kb, 15-125 kb, 25-100 kb, 25-75 kb, 25-50 kb, 50-200 kb, 50-175 kb, 50-150 kb, 50-125 kb, 50-100 kb, 50-75 kb, 75-200 kb, 75-175 kb, 75-150 kb, 75-125 kb, 75-100 kb, 100-200 kb, 100-175 kb, 100-150 kb, 100-125 kb, 125-200 kb, 125-175 kb, 125-150 kb, 150-200 kb, 150-175 kb, or 175-200 kb in length.
   E607. The baculovirus expression construct of any one of embodiments E1-E180, or the variant baculovirus genome of any one of embodiments E292-E606, which comprises at least one non-naturally occurring, e.g., engineered, functional restriction enzyme site (e.g., type II restriction enzyme site (e.g., a type IIS restriction enzyme site).
   E608. The baculovirus expression construct of embodiment E607, or the variant baculovirus genome of embodiment E607, wherein the variant baculovirus nucleotide sequence comprises 1-20, e.g., 1-10, 1-8, 1-6, 1-4, 1, 2, 3, 4, 5, 6, 7, 8, 9, 10, 11, 12, 13, 14, 15, 16, 17, 18, 19, or 20 non-naturally occurring, e.g., engineered, restriction enzyme sites of one or more (e.g., at least about 1-4, 1-3, 1-2, 1, 2, 3, 4, or 5) selected type II restriction enzymes.
   E609. The baculovirus expression construct of embodiment E607 or E608, or the variant baculovirus genome of embodiment E607 or E608, wherein the variant baculovirus nucleotide sequence is devoid of naturally occurring sites for one or more (e.g., about 1-4, 1-3, 1-2, 1, 2, 3, 4, or 5) selected type II restriction enzymes, and comprises at least about 1-20, e.g., at least about 1-10, 1-8, 1-6, 1-4, 1, 2, 3, 4, 5, 6, 7, 8, 9, 10, 11, 12, 13, 14, 15, 16, 17, 18, 19, or 20 non-naturally occurring, e.g., engineered, restriction enzyme sites of the one or more selected type II restriction enzymes.
   E610. The baculovirus expression construct of any one of embodiments E607-E609, or the variant baculovirus genome of any one of embodiments E607-E609, wherein the non-naturally occurring restriction enzyme site is present at a different location compared to the wild-type baculovirus genome.
   E611. The baculovirus expression construct, the variant baculovirus genome, or the plurality of fragments of any one of the preceding embodiments, wherein variant baculovirus nucleotide sequence is chemically synthesized and/or a non-templated nucleotide sequence (e.g., non-templated fragment).
   E612. A vector comprising the baculovirus expression construct of any one of embodiments E1-E180, the plurality of fragments of any one of embodiments E181-E291, or the variant baculovirus genome of any one of embodiments E292-E611.
   E613. The vector of embodiment E612, which is a bacterial artificial chromosome (BAC).
   E614. A bacterial artificial chromosome (BAC) which comprises at least 5 fewer functional restriction enzyme sites (e.g., functional naturally occurring restriction enzyme sites), e.g., type II restriction enzyme sites (e.g., type IIS restriction enzyme sites), relative to a reference BAC, e.g., a wild-type BAC.
   E615. A bacterial artificial chromosome (BAC) which comprises at least 5 fewer functional restriction enzyme sites (e.g., functional naturally occurring restriction enzyme sites), e.g., type II restriction enzyme sites (e.g., type IIS restriction enzyme sites) of one or more (e.g., 1-4, 1-3, 1-2, 1, 2, 3, 4, or 5) selected restriction enzymes (e.g., type II restriction enzymes (e.g., type IIS restriction enzymes) relative to a reference BAC, e.g., a wild-type BAC.
   E616. A vector comprising a baculovirus genome or variant thereof, wherein the vector is a BAC, and wherein the BAC comprises at least 5 fewer restriction enzyme sites (e.g., functional naturally occurring restriction enzyme sites), e.g., type II restriction enzyme sites (e.g., type IIS restriction enzyme sites), relative to a reference BAC, e.g., a wild-type BAC.
   E617. The vector of embodiment E612, E613, or E616, or the BAC of embodiment E614 or E615, wherein the BAC is devoid of type II restriction enzyme sites (e.g., type IIS restriction enzyme sites).
   E618. The vector of embodiment E612, E613, E616, or E617, or the BAC of any one of embodiments E614-E617, wherein the BAC is devoid of recognition sites of a selected restriction enzyme, e.g., type II restriction enzyme (e.g., type IIS restriction enzyme).
   E619. The vector of any one of embodiments E612, E613, or E616-E618, or the BAC of any one of embodiments E614-E618, wherein the BAC is devoid of recognition sites of two or more selected restriction enzymes, e.g., type II restriction enzymes (e.g., type IIS restriction enzymes).
   E620. A cell comprising the baculovirus expression construct of any one of embodiments E1-E180 or E607-E611, plurality of fragments of any one of embodiments E181-E291 or E612, variant baculovirus genome of any one of embodiments E292-E612, vector of embodiment E612, E613, or E616-E619, or BAC ofany one of embodiments E614-E619.
   E621. The cell of embodiment E620 which is an insect cell (e.g., an Sf9 cell or an Sf21), a mammalian cell (e.g., HEK293 cell), or a bacterial cell (e.g., E. coli).
   E622. A composition comprising the baculovirus expression construct of any one of embodiments E1-E180 or E607-E611, plurality of fragments of any one of embodiments E181-E291 or E612, variant baculovirus genome of any one of embodiments E292-E612, vector of embodiment E612, E613, or E616-E619, or BAC of any one of embodiments E614-E619, and a carrier.
   E623. A kit comprising the baculovirus expression construct of any one of embodiments E1-E180 or E607-E611, plurality of fragments of any one of embodiments E181-E291 or E612, variant baculovirus genome of any one of embodiments E292-E612, vector of embodiment E612, E613, or E616-E619, or BAC of any one of embodiments E614-E619, and instructions for use.
   E624. The kit of embodiment E623, which further comprises one or more restriction enzymes (e.g., type II restriction enzymes (e.g., type IIS restriction enzymes)).
   E625. An AAV viral production system comprising the variant baculovirus genome comprising the AAV expression construct of any one of embodiments E292-E612 and the variant baculovirus genome comprising the AAV payload construct of any one of embodiments E597-E602.
   E626. An AAV viral production system comprising the variant baculovirus genome of embodiment E603.
   E627. The AAV viral production system of embodiment E625 or E626, which comprises an AAV viral production cell which comprises the variant baculovirus genome comprising the AAV expression construct and variant baculovirus genome comprising the AAV payload construct.
   E628. The AAV viral production system of embodiment E627, wherein the AAV viral production cell is an insect cell; optionally a Sf9 cell or a Sf21 cell.
   E629. A method of producing recombinant adeno-associated virus (rAAV) particle in an AAV viral production cell, the method comprising: (i) providing an AAV viral production system of any one of embodiments E626-E628, wherein the AAV expression construct comprises one or more VP-coding regions which comprise one or more nucleotide sequences encoding VP1, VP2 and VP3 capsid proteins; (ii) transfecting the AAV viral production system into an AAV viral production cell; (iii) exposing the AAV viral production cell to conditions which allow the AAV viral production cell to process the AAV expression construct and the AAV payload construct into rAAV particles; and, optionally, (iv) collecting the rAAV particles from the AAV viral production cell, e.g., an insect cell such as a Sf9 cell or a Sf21cell.
   E630. A recombinant adeno-associated virus (rAAV) particle produced by the method of embodiment E629.
   E631. A method of producing an AAV particle, the method comprising:
(i) providing a cell comprising the variant baculovirus genome of any one of embodiments E292-E612, or a cell comprising the AAV production system of any one of embodiments E625-E628;
(ii) incubating the cell under conditions suitable to produce the AAV particle;
thereby producing the AAV particle.
   E632. A recombinant adeno-associated virus (rAAV) particle produced by the method of embodiment E631.
   E633. A pharmaceutical composition comprising the rAAV particle of embodiment E630 or E632 and a pharmaceutically acceptable excipient.
   E634. A method of generating a variant baculovirus genome, comprising:
(i) providing a plurality of fragments, e.g., subgenomic fragments or subfragments, wherein each fragment comprises:
   (a) a unique 5′ overhang and 3′ overhang, or a nucleotide sequence at the 5′ end that overlaps with (e.g., is homologous with) the nucleotide sequence at the 3′ end of another fragment, wherein sequence in the region of overlap is unique to a pair of fragments;
   (b) a variant baculovirus nucleotide sequence comprising at least 5 fewer functional restriction enzyme sites (e.g., functional naturally occurring restriction enzyme sites), e.g., type II restriction enzyme sites (e.g., type IIS restriction enzyme sites), relative to a nucleotide sequence in a reference baculovirus genome, e.g., a nucleotide sequence in a wild-type baculovirus genome;
(ii) introducing a modification (e.g., insertion, substitution, or deletion), e.g., one or more modifications, into one or more fragments comprising the variant baculovirus nucleotide sequence; and
(iii) incubating the plurality of fragments under conditions suitable to form a variant baculovirus genome;
thereby generating the variant baculovirus genome.
   E635. A method of generating a variant baculovirus genome, comprising:
(i) providing a plurality of fragments, e.g., subgenomic fragments or subfragments, wherein each fragment comprises:
   (a) a unique 5′ overhang and 3′ overhang, or a nucleotide sequence at the 5′ end that overlaps with (e.g., is homologous with) the nucleotide sequence at the 3′ end of another fragment, wherein sequence in the region of overlap is unique to a pair of fragments;
   (b) a variant baculovirus nucleotide sequence comprising at least 10 fewer functional restriction enzyme sites (e.g., functional naturally occurring restriction enzyme sites), e.g., type II restriction enzyme sites (e.g., type IIS restriction enzyme sites), relative to a nucleotide sequence in a reference baculovirus genome, e.g., a nucleotide sequence in a wild-type baculovirus genome;
wherein one or more fragments of the plurality comprise a modification (e.g., insertion, substitution, or deletion), e.g., one or more modifications, in the variant baculovirus nucleotide sequence; and
(ii) incubating the plurality of fragments under conditions suitable to form a variant baculovirus genome;
thereby generating the variant baculovirus genome.
   E636. The method of embodiment E634 or E635, wherein the variant baculovirus nucleotide sequence comprises at least 10, 15, 20, 25, 30, 35, 40, 45, 50, 55, or 60 fewer, e.g., 15-60, 15-50, 15-40, 15-30, 15-20, 20-60, 20-50, 20-40, 20-30, 30-60, 30-50, 30-40, 40-60, 40-50, or 50-60 fewer, functional restriction enzyme sites (e.g., functional naturally occurring restriction enzyme sites), e.g., type II restriction enzyme sites (e.g., type IIS restriction enzyme sites), relative to a nucleotide sequence in a reference baculovirus genome, e.g., a nucleotide sequence in a wild-type baculovirus genome.
   E637. The method of any one of embodiments E634-E636, wherein the variant baculovirus nucleotide sequence comprises:
(i) at least 10, at least 15, at least 20, at least 25, at least 30, at least 35, at least 40, at least 45, at least 50, at least 55, or at least 60 fewer, e.g., 15-60, 15-50, 15-40, 15-30, 15-20, 20-60, 20-50, 20-40, 20-30, 30-60, 30-50, 30-40, 40-60, 40-50, or 50-60 fewer, functional recognition sites (e.g., functional naturally occurring restriction enzyme sites) of a type II restriction enzyme, e.g., a selected type II restriction enzyme, relative to a nucleotide sequence in a reference baculovirus genome, e.g., a nucleotide sequence in a wild-type baculovirus genome; or
(ii) at least 10, at least 15, at least 20, at least 25, at least 30, at least 35, at least 40, at least 45, at least 50, at least 55, or at least 60 fewer, e.g., 15-60, 15-50, 15-40, 15-30, 15-20, 20-60, 20-50, 20-40, 20-30, 30-60, 30-50, 30-40, 40-60, 40-50, or 50-60 fewer, functional recognition sites (e.g., functional naturally occurring restriction enzyme sites) of two or more (e.g., 2-5, 2, 3, 4, or 5) selected type II restriction enzymes, relative to a nucleotide sequence in a reference baculovirus genome, e.g., a nucleotide sequence in a wild-type baculovirus genome.
   E638. The method of any one of embodiments E634-E637, wherein the variant baculovirus nucleotide sequence comprises at least 10, at least 15, at least 20, at least 25, at least 30, at least 35, at least 40, at least 45, at least 50, at least 55, or at least 60 fewer, e.g., 15-60, 15-50, 15-40, 15-30, 15-20, 20-60, 20-50, 20-40, 20-30, 30-60, 30-50, 30-40, 40-60, 40-50, or 50-60 fewer, functional type IIS restriction enzyme sites (e.g., functional naturally occurring type IIS restriction enzyme sites) relative to a nucleotide sequence in a reference baculovirus genome, e.g., a nucleotide sequence in a wild-type baculovirus genome.
   E639. The method of any one of embodiments E634-E638, wherein the variant baculovirus nucleotide sequence comprises:
(i) at least 10, at least 15, at least 20, at least 25, at least 30, at least 35, at least 40, at least 45, at least 50, at least 55, or at least 60 fewer, e.g., 15-60, 15-50, 15-40, 15-30, 15-20, 20-60, 20-50, 20-40, 20-30, 30-60, 30-50, 30-40, 40-60, 40-50, or 50-60 fewer, functional recognition sites (e.g., functional naturally occurring recognition sites) of a selected type IIS restriction enzyme relative to a nucleotide sequence in a reference baculovirus genome, e.g., a nucleotide sequence in a wild-type baculovirus genome; or
(ii) at least 10, at least 15, at least 20, at least 25, at least 30, at least 35, at least 40, at least 45, at least 50, at least 55, or at least 60 fewer, e.g., 15-60, 15-50, 15-40, 15-30, 15-20, 20-60, 20-50, 20-40, 20-30, 30-60, 30-50, 30-40, 40-60, 40-50, or 50-60 fewer, functional recognition sites (e.g., functional naturally occurring recognition sites) of two or more (e.g., 2-5, 2, 3, 4, or 5) selected type IIS restriction enzymes relative to a nucleotide sequence in a reference baculovirus genome, e.g., a nucleotide sequence in a wild-type baculovirus genome.
   E640. The method of any one of embodiments E634-E639, wherein the variant baculovirus nucleotide sequence comprises:
(i) no functional recognition sites (e.g., functional naturally occurring recognition sites) of two or more (e.g., 2, 3, 4, or 5) selected type II restriction enzymes;
(ii) no functional recognition sites (e.g., functional naturally occurring recognition sites) of a selected type of type II restriction enzyme; or
(iii) no functional recognition sites (e.g., functional naturally occurring recognition sites) for type II restriction enzymes.
   E641. The method of any one of embodiments E634-E640, wherein the variant baculovirus nucleotide sequence comprises no functional recognition sites (e.g., functional naturally occurring recognition sites) of a selected type IIS restriction enzyme.
   E642. The method of any one of embodiments E634-E641, wherein the variant baculovirus nucleotide sequence comprises no functional recognition sites (e.g., functional naturally occurring recognition sites) of two or more (e.g., 2, 3, 4, or 5) selected type IIS restriction enzymes.
   E643. The method of any one of embodiments E634-E642, wherein the variant baculovirus nucleotide sequence comprises no functional recognition sites (e.g., functional naturally occurring recognition sites) for type IIS restriction enzymes.
   E644. The method of any one of embodiments E634-E643, wherein the variant baculovirus nucleotide sequence is devoid of:
(i) recognition sites (e.g., functional naturally occurring recognition sites) of a selected type II restriction enzyme;
(ii) recognition sites (e.g., functional naturally occurring recognition sites) for 1-5 (e.g., 1-4, 1-3, 1-2, 1, 2, 3, 4, or 5) selected type II restriction enzymes; or
(iii) recognition sites (e.g., functional naturally occurring recognition sites) for type II restriction enzymes.
   E645. The method of any one of embodiments E634-E644, wherein the variant baculovirus nucleotide sequence is devoid of type IIS restriction enzyme sites.
   E646. The method of any one of embodiments E634-E645, wherein the type IIS restriction enzyme sites are recognition sites for a selected type IIS restriction enzyme.
   E647. The method of any one of embodiments E634-E646, wherein the type IIS restriction enzyme sites are recognition sites for two or more (e.g., 2, 3, 4, or 5) selected type IIS restriction enzymes.
   E648. The method of any one of embodiments E634-E647, wherein the variant baculovirus nucleotide sequence is devoid of type IIS restriction enzyme sites.
   E649. The method of any one of embodiments E634-E648, wherein the restriction enzyme site is recognized by a restriction enzyme having one, two, three, four or all of the following properties:
(i) is capable of producing a 5′ overhang upon cleavage by the restriction enzyme;
(ii) cleavage by the restriction enzyme results in at least one or at least two (e.g., one or two) base pairs outside the 5′ overhang in its recognition site;
(iii) is capable of producing a 3′ overhang upon cleavage by the restriction enzyme; (iv) is heat inactivatable; and/or
(v) recognizes a stretch of at least 4-8 base pairs, e.g., at least 4 base pairs, at least 5 base pairs, at least 6 base pairs, at least 7 base pairs, or at least 8 base pairs (e.g., 8 base pairs).
   E650. The method of any one of embodiments E634-E649, wherein the restriction enzyme site is a type II restriction enzyme site.
   E651. The method of any one of embodiments E634-E650, wherein the restriction enzyme site is recognized by a restriction enzyme that is able to be used in a Gibson Assembly™ cloning and ligation method (e.g., a method as described in Gibson et al. “Enzymatic assembly of DNA molecules up to several hundred kilobases,” Nat. Methods, 2009, 6(5):343-5; the contents of which are hereby incorporated by reference in their entirety), e.g., a Gibson Assembly™ compatible enzyme.
   E652. The method of any one of embodiments E634-E651, wherein the restriction enzyme site is recognized by a restriction enzyme selected from AcuI, AlwI, AaR1, BaeI, BbsI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, EarI, EciI, Esp3I, FauI, FokI, HgaI, HphI, HpyAV, MbolI, MlyI, MnlI, Nme-AIII, PaqCI, PleI, SapI, SfaNI, AbsI, Acc65I, AgeI, AgsI, ArsI, AscI, AsiGI, AsiSI, Asp718I, AspA2I, AsuNHI, AvrII, Alw44I, AxyI, BamHI, BelI, BcnI, BglII, Bpu1102I, BlnI, BmtI, BsaI, Bse21I, BshTI, BsmBI, Bso31I, BspOI, BspTNI, BstENI, Bsp1191, Bsp120I, Bsu15I, Bsu36I, CciNI, Cfr13I, Csp6I, Cfr9I, CspAI, Ec1136II, Eco31I, Eco53kI, Eco81I, Eco91I, Eco52I, EcoICRI, EcoNI, EcoRI, Esp3I, FseI, Hin6I, HindIII, HpaII, I-CeuI, I-PpoI, I-SceI, KpnI, Kpn2I, LmnI, LpnPI, MauBI, MspI, MluI, NcoI, NdeI, NheI, NotI, PalAI, PI-PspI, PI-SceI, PinAI, Psp124BI, RgaI, RigI, SacI, SalI, SbfI, SdaI, SfaAI, SgfI, SgsI, SmaI, SrfI, Sse8387I, SstI, TaqI, TspMI, XagI, XbaI, XhoI, XmaI, XmaCI, XmaJI, MunI, HinfI, BspTI, Pfl23II, VspI, Bsp1407I, Psp1406I, Tru1I, Bpu101, BcuI, PagI, BshTI, XmaJI, PfoI, SgsI, PteI, KflI, or an isoschizomer of any of the aforesaid restriction enzymes.
   E653. The method of any one of embodiments E634-E652, wherein the restriction enzyme site is a type IIS restriction enzyme site.
   E654. The method of any one of embodiments E634-E653, wherein the type IIS restriction enzyme site is recognized by a restriction enzyme selected from AcuI, AlwI, AaR1, BaeI, BbsI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, EarI, EciI, Esp3I, FauI, FokI, HgaI, HphI, HpyAV, MbolI, MlyI, MnlI, Nme-AIII, PaqCI, PleI, SapI, or SfaNI, or an isoschizomer of any of the aforesaid restriction enzymes.
   E655. The method of any one of embodiments E634-E654, wherein the type IIS restriction enzyme sites are a BsmBI restriction enzyme site, a BsaI restriction enzyme site, a PaqCI restriction enzyme site, or a combination thereof.
   E656. The method of any one of embodiments E634-E655, wherein the type IIS restriction enzyme site is a BsmBI restriction enzyme site.
   E657. The method of any one of embodiments E634-E656, wherein the restriction enzyme site is recognized by a restriction enzyme selected from AbsI, Acc65I, AgeI, AgsI, ArsI, AscI, AsiGI, AsiSI, Asp718I, AspA2I, AsuNHI, AvrII, Alw44I, AxyI, BamHI, BelI, BcnI, BglII, Bpu1102I, BlnI, BmtI, BsaI, Bse21I, BshTI, BsmBI, Bso31I, BspOI, BspTNI, BstENI, Bsp1191, Bsp120I, Bsu15I, Bsu36I, CciNI, Cfr13I, Csp6I, Cfr9I, CspAI, Ec1136II, Eco31I, Eco53kI, Eco81I, Eco91I, Eco52I, EcoICRI, EcoNI, EcoRI, Esp3I, FseI, Hin6I, HindIII, HpaII, I-CeuI, I-PpoI, I-SceI, KpnI, Kpn2I, LmnI, LpnPI, MauBI, MspI, MluI, NcoI, NdeI, NheI, NotI, PalAI, PI-PspI, PI-SceI, PinAI, Psp124BI, RgaI, RigI, SacI, SalI, SbfI, SdaI, SfaAI, SgfI, SgsI, SmaI, SrfI, Sse8387I, SstI, TaqI, TspMI, XagI, XbaI, XhoI, XmaI, XmaCI, XmaJI, MunI, HinfI, BspTI, Pfl23II, VspI, Bsp1407I, Psp1406I, Tru1I, Bpu10I, BcuI, PagI, BshTI, XmaJI, PfoI, SgsI, PteI, KflI, or an isoschizomer of any of the aforesaid restriction enzymes.
   E658. The method of any one of embodiments E634-E657, wherein the restriction enzyme site is recognized by AgeI, AvrII, BamHI, NheI, AscI, NotI, or an isoschizomer of any of the aforesaid restriction enzymes.
   E659. The method of any one of embodiments E634-E658, wherein the restriction enzyme site is recognized by a restriction enzyme selected from AgeI, AvrII, BamHI, NheI, or an isoschizomer of any of the aforesaid restriction enzymes.
   E660. The method of any one of embodiments E634-E658, wherein the restriction enzyme site is recognized by a restriction enzyme selected from AscI, NotI, or an isoschizomer of any of the aforesaid restriction enzymes.
   E661. The method of any one of embodiments E634-E660, wherein the at least one, two, three, four or more of the at least 5 fewer functional type IIS restriction enzyme sites are different type IIS restriction enzyme sites, e.g., a first type IIS restriction enzyme site and a second type IIS restriction enzyme site, optionally wherein:
(i) the first type IIS restriction enzyme site is recognized by a restriction enzyme selected from AcuI, AlwI, AaR1, BaeI, BbsI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, EarI, EciI, Esp3I, FauI, FokI, HgaI, HphI, HpyAV, MbolI, MlyI, MnlI, Nme-AIII, PaqCI, PleI, SapI, or SfaNI, or an isoschizomer of any of the preceding restriction enzymes; and
(ii) the second type IIS restriction enzyme site is recognized by a restriction enzyme selected from AcuI, AlwI, AaR1, BaeI, BbsI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, EarI, EciI, Esp3I, FauI, FokI, HgaI, HphI, HpyAV, MbolI, MlyI, MnlI, Nme-AIII, PaqCI, PleI, SapI, or SfaNI, or an isoschizomer of any of the preceding restriction enzymes.
   E662. The method of any one of embodiments E634-E661, wherein each fragment comprises a different 5′ overhang and/or a 3′ overhang relative to the other fragments of the plurality.
   E663. The method of any one of embodiments E634-E662, wherein each fragment is capable of ordered assembly based on complementarity of the unique 5′ overhang in one fragment with the unique 3′ overhang in another fragment.
   E664. The method of any one of embodiments E634-E663, wherein the unique 5′ overhang of one fragment of the plurality is complementary to the unique 3′ overhang of another fragment of the plurality.
   E665. The method of any one of embodiments E634-E664, wherein the unique 5′ overhang is partially complementary or fully complementary to the unique 3′ overhang.
   E666. The method of any one of embodiments E634-E665, wherein each fragment of the plurality comprises a nucleotide sequence at the 5′ end that overlaps with (e.g., is homologous with) the nucleotide sequence at the 3′ end of another fragment of the plurality, wherein the region of overlap is unique to a pair of fragments.
   E667. The method of any one of embodiments E634-E666, wherein fragments of the plurality are capable of ordered assembly based on the overlap in nucleotide sequence at the 5′ end of one fragment (e.g., subgenomic fragment or subfragment) with the nucleotide sequence at the 3′ end of another fragment (e.g., subgenomic fragment or subfragment) to generate a variant baculovirus genome or the baculovirus expression construct.
   E668. The method of embodiment E666 or E667, wherein the overlap in nucleotide sequence between the fragments is 4-50 (e.g., 4-45, 4-40, 4-35, 4-30, 4-25, 4-20, 4-15, 4-10, 10-50, 10-45, 10-40, 10-35, 10-30, 10-25, 10-20, 10-15, 15-50, 15-45, 15-40, 15-35, 15-30, 15-25, 15-20, 20-50, 20-45, 20-40, 20-35, 20-30, 20-25, 25-50, 25-45, 25-40, 25-35, 25-30, 30-50, 30-45, 30-40, 30-35, 35-50, 35-45, 35-40, 40-50, 40-45) contiguous base pairs.
   E669. The method of any one of embodiments E634-E668, wherein the unique 5′ overhang and unique 3′ overhang result from cleavage of the fragment by a restriction enzyme, e.g., a type II restriction enzyme (e.g., a type IIS restriction enzyme).
   E670. The method of any one of embodiments E634-E669, wherein the 5′ and 3′ overhang each independently comprise 1-6 nucleotides in length, e.g., 2-5 nucleotides (e.g., 4 nucleotides).
   E671. The method of any one of embodiments E634-E670, wherein the unique 5′ and 3′ overhang each independently comprise 4 nucleotides.
   E672. The method of any one of embodiments E634-E671, wherein the unique 5′ overhang and unique 3′ overhang of the fragments are each independently selected from ACAA, GGTC, GACC, CCAG, CTGG, CCTT, AAGG, TCAT, ATGA, TCGC, GCGA, AGAG, CTCT, AACT, AGTT, CGGT, ACCG, ATAC, GTAT, GAGT, ACTC, TTCC, GGAA, ATTA, TAAT, TCCT, AGGA, TCTA, TAGA, TGTA, TACA, GATG, CATC, or TTGT, wherein the sequences of the unique 5′ overhang and 3′ overhang in a fragment are different.
   E673. The method of any one of embodiments E634-E672, wherein the restriction enzyme site is a BsaI restriction enzyme site or a BsmBI restriction enzyme site.
   E674. The method of any one of embodiments E634-E673, wherein:
(i) the subgenomic fragments result from cleavage with a first restriction enzyme, e.g., a first type II restriction enzyme (e.g., a first type IIS restriction enzyme); and/or
(ii) the subfragments result from cleavage with a second restriction enzyme, e.g., a second type II restriction enzyme (e.g., a second type IIS restriction enzyme);
wherein the first restriction enzyme, e.g., a first type II restriction enzyme (e.g., a first type IIS restriction enzyme) is different from the second restriction enzyme, e.g., a second type II restriction enzyme (e.g., a second type IIS restriction enzyme).
   E675. The method of any one of embodiments E634-E674, wherein the one or more fragments of the plurality are capable of ordered assembly based on the complementarity of the unique 5′ overhang in one fragment (e.g., subgenomic fragment or subfragment) of the plurality with the unique 3′ overhang in another fragment (e.g., subgenomic fragment or subfragment) of the plurality to generate a variant baculovirus genome.
   E676. The method of embodiment E674 or E675, wherein the restriction enzyme, the first restriction enzyme and/or the second restriction enzyme comprises one, two, three, four, or all of the following properties:
(i) is capable of producing a 5′ overhang upon cleavage by the restriction enzyme;
(ii) cleavage by the restriction enzyme results in at least one or at least two (e.g., one or two) base pairs outside the 5′ overhang in its recognition site;
(iii) is capable of producing a 3′ overhang upon cleavage by the restriction enzyme;
(iv) is heat inactivatable; and/or
(v) recognizes a stretch of at least 4-8 base pairs, e.g., at least 4 base pairs, at least 5 base pairs, at least 6 base pairs, at least 7 base pairs, or at least 8 base pairs (e.g., 8 base pairs).
   E677. The method of any one of embodiments E674-E676, wherein the first restriction enzyme and/or second restriction enzyme is a type II restriction enzyme.
   E678. The method of any one of embodiments E674-E677, wherein the restriction enzyme, the first restriction enzyme and/or the second restriction enzyme is a restriction enzyme that is able to be used in a Gibson Assembly™ cloning and ligation method (e.g., a method as described in Gibson et al. “Enzymatic assembly of DNA molecules up to several hundred kilobases,” Nat. Methods, 2009, 6(5):343-5; the contents of which are hereby incorporated by reference in their entirety), e.g., a Gibson Assembly™ compatible enzyme.
   E679. The method of any one of embodiments E674-E678, wherein the restriction enzyme, the first restriction enzyme and/or the second restriction enzyme is selected from AcuI, AlwI, AaR1, BaeI, BbsI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, EarI, EciI, Esp3I, FauI, FokI, HgaI, HphI, HpyAV, MbolI, MlyI, MnlI, Nme-AIII, PaqCI, PleI, SapI, SfaNI, AbsI, Acc65I, AgeI, AgsI, ArsI, AscI, AsiGI, AsiSI, Asp718I, AspA2I, AsuNHI, AvrII, Alw44I, AxyI, BamHI, BelI, BcnI, BglII, Bpu1102I, BlnI, BmtI, BsaI, Bse21I, BshTI, BsmBI, Bso31I, BspOI, BspTNI, BstENI, Bsp1191, Bsp120I, Bsu15I, Bsu36I, CciNI, Cfr13I, Csp6I, Cfr9I, CspAI, Ec1136II, Eco31I, Eco53kI, Eco81I, Eco91I, Eco52I, EcoICRI, EcoNI, EcoRI, Esp3I, FseI, Hin6I, HindIII, HpaII, I-CeuI, I-PpoI, I-SceI, KpnI, Kpn2I, LmnI, LpnPI, MauBI, MspI, MluI, NcoI, NdeI, NheI, NotI, PalAI, PI-PspI, PI-SceI, PinAI, Psp124BI, RgaI, RigI, SacI, SalI, SbfI, SdaI, SfaAI, SgfI, SgsI, SmaI, SrfI, Sse8387I, SstI, TaqI, TspMI, XagI, XbaI, XhoI, XmaI, XmaCI, XmaJI, MunI, HinfI, BspTI, Pfl23II, VspI, Bsp1407I, Psp1406I, Tru1I, Bpu10I, BcuI, PagI, BshTI, XmaJI, PfoI, SgsI, PteI, KflI, or an isoschizomer of any of the aforesaid restriction enzymes.
   E680. The method of any one of embodiments E674-E679, wherein the restriction enzyme, the first restriction enzyme and/or the second restriction enzyme is a type IIS restriction enzyme.
   E681. The method of embodiment E680, wherein the type IIS restriction enzyme is selected from AcuI, AlwI, AaR1, BaeI, BbsI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, EarI, EciI, Esp3I, FauI, FokI, HgaI, HphI, HpyAV, MbolI, MlyI, MnlI, Nme-AIII, PaqCI, PleI, SapI, or SfaNI, or an isoschizomer of any of the aforesaid restriction enzymes.
   E682. The method of embodiment E680 or E681, wherein the type IIS restriction enzyme is a BsaI restriction enzyme, a BsmBI restriction enzyme, a PaqCI restriction enzyme, or a combination thereof.
   E683. The method of any one of embodiments E674-E682, wherein:
(i) the first restriction enzyme is BsmBI and the second restriction enzyme is BsaI; or
(ii) the first restriction enzyme is BsaI and the second restriction enzyme is BsmBI.
   E684. The method of any one of embodiments E674-E683, wherein the restriction enzyme, the first restriction enzyme and/or second restriction enzyme is selected from AbsI, Acc65I, AgeI, AgsI, ArsI, AscI, AsiGI, AsiSI, Asp718I, AspA2I, AsuNHI, AvrII, Alw44I, AxyI, BamHI, BelI, BcnI, BglII, Bpu1102I, BlnI, BmtI, BsaI, Bse21I, BshTI, BsmBI, Bso31I, BspOI, BspTNI, BstENI, Bsp1191, Bsp120I, Bsu15I, Bsu36I, CciNI, Cfr13I, Csp6I, Cfr9I, CspAI, Ec1136II, Eco31I, Eco53kI, Eco81I, Eco91I, Eco52I, EcoICRI, EcoNI, EcoRI, Esp3I, FseI, Hin6I, HindIII, HpaII, I-CeuI, I-PpoI, I-SceI, KpnI, Kpn2I, LmnI, LpnPI, MauBI, MspI, MluI, NcoI, NdeI, NheI, NotI, PalAI, PI-PspI, PI-SceI, PinAI, Psp124BI, RgaI, RigI, SacI, SalI, SbfI, SdaI, SfaAI, SgfI, SgsI, SmaI, SrfI, Sse8387I, SstI, TaqI, TspMI, XagI, XbaI, XhoI, XmaI, XmaCI, XmaJI, MunI, HinfI, BspTI, Pfl23II, VspI, Bsp1407I, Psp1406I, Tru1I, Bpu101, BcuI, PagI, BshTI, XmaJI, PfoI, SgsI, PteI, KflI, or an isoschizomer of any of the aforesaid restriction enzymes.
   E685. The method of any one of embodiments E674-E684, wherein the restriction enzyme, the first restriction enzyme and/or the second restriction enzyme is AgeI, AvrII, BamHI, NheI, AscI, NotI, or an isoschizomer of any of the aforesaid restriction enzymes.
   E686. The method of any one of embodiments E674-E685, wherein the restriction enzyme, the first restriction enzyme and/or the second restriction enzyme is AgeI, AvrII, BamHI, NheI, or an isoschizomer of any of the aforesaid restriction enzymes.
   E687. The method of any one of embodiments E674-E685, wherein the restriction enzyme, the first restriction enzyme and/or the second restriction enzyme is AscI, NotI, or an isoschizomer of any of the aforesaid restriction enzymes.
   E688. The method of any one of embodiments E674-E687, wherein the one or more fragments of the plurality are capable of ordered assembly based on the overlap in nucleotide sequence at the 5′ end in one fragment of the plurality with the nucleotide sequence at the 3′ end of another fragment to generate a variant baculovirus genome.
   E689. The method of embodiment E688, wherein the overlap in nucleotide sequence between the fragments is 4-50 (e.g., 4-45, 4-40, 4-35, 4-30, 4-25, 4-20, 4-15, 4-10, 10-50, 10-45, 10-40, 10-35, 10-30, 10-25, 10-20, 10-15, 15-50, 15-45, 15-40, 15-35, 15-30, 15-25, 15-20, 20-50, 20-45, 20-40, 20-35, 20-30, 20-25, 25-50, 25-45, 25-40, 25-35, 25-30, 30-50, 30-45, 30-40, 30-35, 35-50, 35-45, 35-40, 40-50, 40-45) contiguous base pairs.
   E690. The method of embodiment E689, wherein a 5′ exonuclease creates a single-stranded region of complementarity corresponding to the overlapping sequence between fragments, producing fragments capable of annealing with each other.
   E691. The method of embodiment E690, wherein any gaps between the annealed fragments are filled in by a DNA polymerase.
   E692. The method of any one of embodiments E634-E691, wherein the one or more fragments of the plurality are covalently linked, e.g., using a ligase (e.g., a T4 ligase), for example using Golden Gate Assembly or Gibson Assembly™, such that a variant baculovirus genome is formed, optionally wherein the fragments of the plurality are ligated in a single step to form the variant baculovirus genome.
   E693. The method of any one of embodiments E634-E692, wherein each subfragment of the plurality is between 50-1000 bp, e.g., about 50-900, 50-800, 50-700, 50-600, 50-500, 50-400, 50-300, 50-200, 50-100, 100-900, 100-800, 100-700, 100-600, 100-500, 100-400, 100-300, 100-200, 200-1000, 200-900, 200-800, 200-700, 200-600, 200-500, 200-400, 200-300, 400-1000, 400-900, 400-800, 400-700, 400-600, 400-500, 600-1000, 600-900, 600-800, 600-700, 800-1000, 800-900, or 900-1000 bp (e.g., 100-800 bp, about 250-750 bp, about 400-600 bp, or about 500 bp) in length.
   E694. The method of any one of embodiments E634-E693, wherein each subgenomic fragment of the plurality is between 100-25000 bp in length (e.g., about 100-1000 bp, about 100-10000, about 100-20000, about 100-25000, about 1000-10000 bp, about 1000-8000 bp, about 1000-5000 bp, about 1000-2500 bp, about 2500-25000 bp, about 2500-20000 bp, about 2500-15000 bp, about 2500-10000 bp, about 2500-5000 bp, about 5000-25000 bp, about 5000-20000 bp, about 5000-15000 bp, about 5000-10000 bp, about 7500-25000 bp, about 7500-20000, about 7500-15000 bp, about 7500-10000 bp, about 10000-25000 bp, about 10000-20000 bp, about 10000-15000 bp, about 15000-25000 bp, about 15000-20000 bp, about 20000-25000 bp, about 7000-9000 bp, or about 8000 bp in length).
   E695. The method of any one of embodiments E634-E694, wherein the variant baculovirus nucleotide sequence of one or more of the fragments of the plurality comprises a modification, e.g., an insertion, deletion, or substitution.
   E696. The method of embodiment E695, wherein the modification, e.g., an insertion, deletion, substitution, or mutation (e.g., frame-shift mutation), is present in a non-essential gene (e.g., auxiliary and/or per os infectivity factor gene) or a regulatory region of a non-essential gene (e.g., promoter modification or insertion of heterologous DNA adjacent to non-essential gene).
   E697. The method of embodiment E696, wherein the non-essential gene is selected from one, two, three, or more (e.g., 1, 2, 3, 4, 5, 6, 7, 8, 9, 10, 11, 12, 13, 14, 15, 16, 17, 18, 19, 20, or 21) of egt, p74 (PIF0), p26, SOD, ChiA, v-cath, p10, polyhedrin, ctx, odv-e56, PIF1, PIF2, PIF3, PIF4, PIF5, Tn7, AcORF-91, AcORF-108, AcORF-52, v-ubi, or p94.
   E698. The method of any one of embodiments E695-E697, which comprises a modification, e.g., an insertion, deletion, substitution, or mutation (e.g., frame-shift mutation) in:
(i) v-cath and egt;
(ii) v-cath, egt, and SOD;
(iii) chiA, v-cath, egt, p26, p10, and p74;
(iv) chiA, v-cath, egt, p26, p10, p74, and SOD; or
(v) chiA, v-cath, egt, p26, p10, p74, SOD, AcORF-91, and AcORF-108.
   E699. The method of any one of embodiments E695-E698, wherein the modification comprises a deletion of a chiA gene, a v-cath gene, a p26 gene, a p10 gene, and/or a p74 gene, or a portion thereof.
   E700. The method of any one of embodiments E695-E699, wherein the modification comprises an insertion of a heterologous sequence in the non-essential gene or adjacent region.
   E701. The method of any one of embodiments E695-E700, wherein the modification comprises one or more mutations in the non-essential gene or adjacent region.
   E702. The method of any one of embodiments E695-E701, wherein the non-essential genes are present near (e.g., downstream or upstream) of a homologous repeat region 5 (hr5).
   E703. The method of any one of embodiments E695-E702, wherein the modification, e.g., an insertion, deletion, substitution, or mutation (e.g., frame-shift mutation), results in inactivation of a non-essential gene (e.g., auxiliary and/or per os infectivity factor gene) or a regulatory region of a non-essential gene (e.g., promoter modification or insertion of heterologous DNA adjacent to non-essential gene).
   E704. The method of any one of embodiments E695-E703, wherein the variant baculovirus nucleotide sequence of one or more of the fragments of the plurality comprises a disruption, e.g., a mutation (e.g., frameshift mutation), a deletion, an insertion, or inactivation, of a non-essential gene (e.g., auxiliary and/or per os infectivity factor gene) or a regulatory region of a non-essential gene (e.g., promoter modification or insertion of heterologous DNA adjacent to non-essential gene).
   E705. The method of any one of embodiments E695-E704, wherein the modification is introduced by chemical synthesis or PCR, e.g., PCR-based site-directed mutagenesis.
   E706. The method of any one of embodiments E695-E705, wherein one or more of the fragments of the plurality comprises a heterologous nucleotide sequence.
   E707. The method of embodiment E706, wherein the heterologous nucleotide sequence comprises a sequence of interest (e.g., a nucleotide sequence encoding a Rep-coding region, one or more Rep proteins, a VP-coding region, one or more VP proteins, and/or a payload).
   E708. The method of embodiment E707, wherein the sequence of interest is a nucleotide sequence encoding a polypeptide of interest or a nucleotide sequence of interest.
   E709. The method of embodiment E707 or E708, wherein the sequence of interest is operably linked to a first promoter (e.g., a baculovirus early promoter or a baculovirus early-late promoter) and/or a second promoter (e.g., a baculovirus late promoter or a baculovirus very late promoter).
   E710. The method of any one of embodiments E707-E709, wherein the sequence of interest is operably linked to a first promoter (e.g., a baculovirus early promoter or a baculovirus early-late promoter) and a second promoter (e.g., a baculovirus late promoter or a baculovirus very late promoter).
   E711. The method of embodiment E709 or E710, wherein the first and/or second promoter is selected from a baculovirus promoter, a viral promoter, an insect viral promoter, a non-insect viral promoter, a vertebrate viral promoter, a chimeric promoter from one or more species including virus and non-virus elements, a synthetic promoter, or a variant thereof.
   E712. The method of any one of embodiments E709-E711, wherein the first and/or second promoter chosen from a polh promoter, a p10 promoter, a ctx promoter, a gp64 promoter, an IE promoter, an IE-1 promoter, a p6.9 promoter, a Dmhsp70 promoter, a Hsp70 promoter, a p5 promoter, a p19 promoter, a p35 promoter, a p40 promoter, or a variant, e.g., functional fragment, thereof.
   E713. The method of any one of embodiments E709-E712, wherein the first and/or second promoter is selected from an a baculovirus early promoter, baculovirus late promoter, baculovirus early-late promoter, or a baculovirus very late promoter.
   E714. The method of any one of embodiments E709-E713, wherein first and/or second promoter is a baculovirus early promoter, baculovirus late promoter, or baculovirus early-late promoter.
   E715. The method of any one of embodiments E709-E714, wherein first and/or second promoter is a baculovirus early-late promoter (e.g., a gp64 promoter).
   E716. The method of any one of embodiments E709-E715, wherein the first or second promoter is a baculovirus very late promoter (e.g., a polh promoter).
   E717. The method of any one of embodiments E709-E716, wherein:
(a) the first promoter is an baculovirus early-late promoter and the second promoter is a baculovirus very late promoter,
(b) the first promoter is a baculovirus very late promoter and the second promoter is a baculovirus early-late promoter,
(c) the first promoter is a baculovirus early promoter and the second promoter is a baculovirus early-late promoter,
(d) the first promoter is a baculovirus early-late promoter and the second promoter is a baculovirus early promoter,
(e) the first promoter is a baculovirus early promoter and the second promoter is a baculovirus late promoter,
(f) the first promoter is a baculovirus late promoter and the second promoter is a baculovirus early promoter,
(g) the first promoter is a baculovirus early-late promoter and the second promoter is a baculovirus late promoter,
(h) the first promoter is a baculovirus late promoter and the second promoter is a baculovirus early-late promoter,
(i) the first promoter is a baculovirus late promoter and the second promoter is a baculovirus very-late promoter,
(j) the first promoter is a baculovirus very-late promoter and the second promoter is a baculovirus late promoter,
(k) the first promoter is a baculovirus early promoter and the second promoter is a baculovirus very late promoter,
(l) the first promoter is a baculovirus very late promoter and the second promoter is a baculovirus early promoter,
(m) the first promoter is a baculovirus early promoter and the second promoter is a baculovirus early promoter,
(n) the first promoter is a baculovirus early-late promoter and the second promoter is a baculovirus early-late promoter, or
(o) the first promoter is a baculovirus late promoter and the second promoter is a baculovirus late promoter.
   E718. The method of any one of embodiments E709-E717, wherein the first promoter is a baculovirus early-late promoter (e.g., gp64 promoter) and the second promoter is a baculovirus very late promoter (e.g., polh promoter).
   E719. The method of any one of embodiments E709-E718, wherein the first promoter is a baculovirus early promoter and the second promoter is a baculovirus late promoter.
   E720. The method of any one of embodiments E709-E719, wherein
(a) the baculovirus early promoter is selected from: a lef3 promoter, a dbp promoter, a p35 promoter, an orf82 promoter, an get promoter, an orf81 promoter, an orf122 promoter, a pk-2 promoter, an orf55 promoter, an etl promoter, a hcf-1 promoter, an etm promoter, a lef-2 promoter, a lef-6 promoter, an orf84 promoter, an orf118 promoter, or an orf111 promoter,
(b) the baculovirus early-late promoter is selected from: a lef2 promoter, a orf13 promoter, a orf23 promoter, a pkip promoter, a v-fgf promoter, a pp31 promoter, an odv-e66 promoter, an orf74 promoter, an orf79 promoter, an orf82 promoter, a p15 promoter, a cg30 promoter, a helicase promoter, an he65 promoter, an orf114 promoter, a pk-2 promoter, a gp64 promoter, a gp16 promoter, an alk-exo promoter, a p35 promoter, a me53 promoter, or an ie0 promoter,
(c) the baculovirus late promoter is selected from: a ptpase promoter, an Ac-bro promoter, a ctx promoter, an orf5 promoter, an orf19 promoter, an orf20 promoter, an sod promoter, a HisP promoter, an orf34 promoter, a v-ubi promoter, an orf38 promoter, an orf43 promoter, an orf44 promoter, an orf56 promoter, an orf59 promoter, an orf60 promoter, or an fp-25k promoter, and/or
(d) the baculovirus very late promoter is selected from a p10 promoter or a polh promoter.
   E721. The method of any one of embodiments E709-E720, wherein the first and/or second promoter comprises a TATA box motif and/or a CAGT motif.
   E722. The method of any one of embodiments E709-E721, wherein the first and/or second promoter comprises a TAAG motif (e.g., an ATAAG nucleotide sequence).
   E723. The method of any one of embodiments E709-E722, wherein the first and/or second promoter comprises both a TATA box motif and a TAAG motif.
   E724. The method of any one of embodiments E709-E723, wherein the first or second promoter comprises a binding site for VLF-1.
   E725. The method of any one of embodiments E709-E724, wherein the first or second promoter is a gp64 promoter (e.g., an OpMNPV gp64 promoter).
   E726. The method of any one of embodiments E709-E725, wherein the first or second promoter is a polh promoter (e.g., an OpMNPV polh promoter or an AcMNPV polh promoter).
   E727. The method of any one of embodiments E709-E72669, wherein the first promoter is a gp64 promoter and the second promoter is a polh promoter, or wherein the first promoter is a polh promoter and the second promoter is a gp64 promoter.
   E728. The method of any one of embodiments E709-E727, wherein the Rep-coding region is operably linked to a first promoter which is a baculovirus early-late promoter and a second promoter which is baculovirus very late promoter, e.g., a gp64 promoter and a polh promoter, optionally, wherein the Rep-coding region is present downstream of a homologous repeat region hr5.
   E729. The method of any one of embodiments E709-E728, wherein the first promoter is a gp64 promoter and the second promoter is a polh promoter.
   E730. The method of embodiment E709-E717 or E720-E726, wherein the first promoter and the second promoter are the same.
   E731. The method of embodiment E709-E730, wherein the first promoter and the second promoter are different.
   E732. The method of embodiment E730, the first promoter and the second promoter are each a polh promoter.
   E733. The method of embodiment E712-E731, wherein the gp64 promoter comprises the nucleotide sequence of SEQ ID NO: 217; a nucleotide sequence at least 70%, 75%, 80%, 85%, 90%, 95%, 96%, 97%, 98%, or 99% identical thereto; a nucleotide sequence comprising at least one, two, three, four, five, six, or seven, but no more than ten different nucleotides relative to SEQ ID NO: 217; or a nucleotide sequence comprising at least one, two, three, four, five, six, or seven, but no more than ten modifications (e.g., substitutions) relative to SEQ ID NO: 217.
   E734. The method of embodiment E712-E732, wherein the polh promoter comprises the nucleotide sequence of SEQ ID NO: 167 or 220; a nucleotide sequence at least 70%, 75%, 80%, 85%, 90%, 95%, 96%, 97%, 98%, or 99% identical thereto; a nucleotide sequence comprising at least one, two, three, four, five, six, or seven, but no more than ten different nucleotides relative to SEQ ID NO: 167 or 220; or a nucleotide sequence comprising at least one, two, three, four, five, six, or seven, but no more than ten modifications (e.g., substitutions) relative to SEQ ID NO: 167 or 220.
   E735. The method of any one of embodiments E712-E732, wherein the first promoter and the second promoter comprises the nucleotide sequence of SEQ ID NO: 221; a nucleotide sequence at least 70%, 75%, 80%, 85%, 90%, 95%, 96%, 97%, 98%, or 99% identical thereto; a nucleotide sequence comprising at least one, two, three, four, five, six, or seven, but no more than ten different nucleotides relative to SEQ ID NO: 221; or a nucleotide sequence comprising at least one, two, three, four, five, six, or seven, but no more than ten modifications (e.g., substitutions) relative to SEQ ID NO: 221.
   E736. The method of any one of embodiments E707-E735, wherein the sequence of interest encodes a therapeutic protein or functional variant thereof, an antibody or antibody fragment; an enzyme; a component of a gene editing system; an RNAi agent (e.g., a dsRNA, siRNA, shRNA, pre-miRNA, pri-miRNA, miRNA, stRNA, lncRNA, piRNA, or snoRNA); or a combination thereof.
   E737. The method of any one of embodiments E634-E736, wherein one or more of the fragments of the plurality (e.g., subgenomic fragments or subfragments) encodes one or more AAV proteins.
   E738. The method of any one of embodiments E634-E737, wherein one or more of the fragments of the plurality (e.g., subgenomic fragments or subfragments) encodes an AAV Rep protein, e.g., Rep40, Rep52, Rep68, Rep78, or a combination thereof.
   E739. The method of any one of embodiments E634-E738, wherein one or more of the fragments of the plurality (e.g., subgenomic fragments or subfragments) encodes a Rep78 protein and/or a Rep52 protein.
   E740. The method of any one of embodiments E634-E739, wherein one or more of the fragments of the plurality (e.g., subgenomic fragments or subfragments) encodes an AAV capsid protein, e.g., a VP1 protein, a VP2 protein, a VP3, protein or a combination thereof.
   E741. The method of any one of embodiments E634-E740, wherein one or more of the fragments of the plurality (subgenomic fragments or subfragments) encodes an AAV1 capsid protein, an AAV2 capsid protein, an AAV3 capsid protein, an AAV4 capsid protein, an AAV5 capsid protein, an AAV6 capsid protein, an AAV8 capsid protein, an AAV9 capsid protein, an AAVrh10 capsid protein, or a variant thereof.
   E742. The method of any one of embodiments E634-E741, wherein one or more of the fragments of the plurality (e.g., subgenomic fragments or subfragments) encodes an AAV5 capsid protein or variant thereof, or an AAV9 capsid protein or variant thereof.
   E743. The method of any one of embodiments E634-E742, wherein one or more of the fragments of the plurality (e.g., subgenomic fragments or subfragments) encodes a payload.
   E744. The method of embodiment E743, wherein the encoded payload is selected from a therapeutic protein or functional variant thereof; an antibody or antibody fragment; an enzyme; a component of a gene editing system; an RNAi agent (e.g., a dsRNA, siRNA, shRNA, pre-miRNA, pri-miRNA, miRNA, stRNA, lncRNA, piRNA, or snoRNA); or a combination thereof.
   E745. The method of any one of embodiments E634-E744, wherein one or more of the fragments of the plurality (e.g., subgenomic region or subfragments) are chemically synthesized or are non-templated fragments.
   E746. The method of any one of embodiments E634-E745, wherein the variant baculovirus genome is capable of producing at least 70% (e.g., at least 80%, at least 90%, at least 100%, at least 110%, at least 120%, 70-200%, 70-150%, 70-100%, 80-200%, 80-150%, 80-100%, 90-200%, 90-150%, 90-100%, 100-200%, 100-150%, 100-125%, 100-110%, 125-200%, 150-200%, or 175-200%) of the amount of baculovirus produced by a reference baculovirus genome (e.g., a wild-type baculovirus genome), e.g., as assessed by viral titer (e.g., using qPCR).
   E747. The method of any one of embodiments E634-E746, wherein the variant baculovirus genome is 10 kb-200 kb in length, e.g., at least 10 kb, 20 kb, 30 kb, 40 kb, 50 kb, 60 kb, 70 kb, 80 kb, 90 kb, 100 kb, 110 kb, 120 kb, 130 kb, or 140 kb, or 10-175 kb, 10-150 kb, 10-125 kb, 10-100 kb, 10-75 kb, 10-50 kb, 10-25 kb, 25-200 kb, 25-175 kb, 25-150 kb, 15-125 kb, 25-100 kb, 25-75 kb, 25-50 kb, 50-200 kb, 50-175 kb, 50-150 kb, 50-125 kb, 50-100 kb, 50-75 kb, 75-200 kb, 75-175 kb, 75-150 kb, 75-125 kb, 75-100 kb, 100-200 kb, 100-175 kb, 100-150 kb, 100-125 kb, 125-200 kb, 125-175 kb, 125-150 kb, 150-200 kb, 150-175 kb, or 175-200 kb in length.
   E748. A method of producing a plurality of subgenomic fragments capable of assembly into a variant baculovirus genome comprising:
(i) providing a reference, e.g., parental, baculovirus genome;
(ii) optionally, identifying one or more sites, e.g., all recognition sites, recognized by a restriction enzyme, e.g., a type II restriction enzyme (e.g., a type IIS restriction enzyme), in the reference baculovirus genome,
(iii) modifying the one or more recognition sites such that the baculovirus genome comprises at least 5 fewer functional restriction enzyme sites, e.g., type II restriction enzyme sites (e.g., type IIS restriction enzyme sites), relative to a nucleotide sequence in a reference baculovirus genome, e.g., a nucleotide sequence in a wild-type baculovirus genome, thereby generating a variant baculovirus genome,
(iv) partitioning the primary template into the plurality of subgenomic fragments, wherein each subgenomic fragment of the plurality comprises (a) a unique 5′ overhang and a unique 3′ overhang, and wherein the subgenomic fragments are capable of ordered assembly based on complementarity of the 5′ overhang in one subgenomic fragment with the 3′ overhang in another subgenomic fragment or (b) a nucleotide sequence at the 5′ end that overlaps with (e.g., is homologous with) the nucleotide sequence at the 3′ end of another fragment, wherein sequence in the region of overlap is unique to a pair of fragments;
thereby producing the plurality of subgenomic fragments.
   E749. The method of embodiment E748, wherein the reference baculovirus genome is a genome of a baculovirus selected from Autographa californica multiple nucleopolyhedrovirus (AcMNPV) (e.g., an AcMNPV strain E2, C6 or HR3), Bombyx mori nucleopolyhedrovirus (BmNPV), Anticarsia gemmatalis nucleopolyhedrovirus (AgMNPV), Orgyia pseudotsugata nucleopolyhedrovirus (OpMNPV), Thysanoplusia orichalcea nucleopolyhedrovirus (ThorMNPV), or a variant thereof.
   E750. The method of embodiment E748 or E749, wherein the variant baculovirus genome comprises at least 10, 15, 20, 25, 30, 35, 40, 45, 50, 55, or 60 fewer, e.g., 15-60, 15-50, 15-40, 15-30, 15-20, 20-60, 20-50, 20-40, 20-30, 30-60, 30-50, 30-40, 40-60, 40-50, or 50-60 fewer, functional restriction enzyme sites, (e.g., functional naturally occurring restriction enzyme sites), e.g., type II restriction enzyme sites (e.g., type IIS restriction enzyme sites), relative to a nucleotide sequence in a reference baculovirus genome, e.g., a nucleotide sequence in a wild-type baculovirus genome.
   E751. The method of any one of embodiments E748-E750, wherein the variant baculovirus genome comprises:
(i) at least 10, at least 15, at least 20, at least 25, at least 30, at least 35, at least 40, at least 45, at least 50, at least 55, or at least 60 fewer, e.g., 15-60, 15-50, 15-40, 15-30, 15-20, 20-60, 20-50, 20-40, 20-30, 30-60, 30-50, 30-40, 40-60, 40-50, or 50-60 fewer, functional recognition sites (e.g., functional naturally occurring recognition sites) of a type II restriction enzyme, e.g., a selected type II restriction enzyme relative to a nucleotide sequence in a reference baculovirus genome, e.g., a nucleotide sequence in a wild-type baculovirus genome; or
(ii) at least 10, at least 15, at least 20, at least 25, at least 30, at least 35, at least 40, at least 45, at least 50, at least 55, or at least 60 fewer, e.g., 15-60, 15-50, 15-40, 15-30, 15-20, 20-60, 20-50, 20-40, 20-30, 30-60, 30-50, 30-40, 40-60, 40-50, or 50-60 fewer, functional recognition sites (e.g., functional naturally occurring recognition sites) of two or more (e.g., 2-5, 2, 3, 4, or 5) selected type II restriction enzymes relative to a nucleotide sequence in a reference baculovirus genome, e.g., a nucleotide sequence in a wild-type baculovirus genome.
   E752. The method of any one of embodiments E748-E751, wherein the variant baculovirus genome comprises at least 10, at least 15, at least 20, at least 25, at least 30, at least 35, at least 40, at least 45, at least 50, at least 55, or at least 60 fewer, e.g., 15-60, 15-50, 15-40, 15-30, 15-20, 20-60, 20-50, 20-40, 20-30, 30-60, 30-50, 30-40, 40-60, 40-50, or 50-60 fewer, functional type IIS restriction enzyme sites (e.g., functional naturally occurring type IIS restriction enzyme sites) relative to a nucleotide sequence in a reference baculovirus genome, e.g., a nucleotide sequence in a wild-type baculovirus genome.
   E753. The method of any one of embodiments E748-E752, wherein the variant baculovirus genome comprises:
(i) at least 10, at least 15, at least 20, at least 25, at least 30, at least 35, at least 40, at least 45, at least 50, at least 55, or at least 60 fewer, e.g., 15-60, 15-50, 15-40, 15-30, 15-20, 20-60, 20-50, 20-40, 20-30, 30-60, 30-50, 30-40, 40-60, 40-50, or 50-60 fewer, functional recognition sites (e.g., functional naturally occurring recognition sites) of a selected type IIS restriction enzyme relative to a nucleotide sequence in a reference baculovirus genome, e.g., a nucleotide sequence in a wild-type baculovirus genome; or
(ii) at least 10, at least 15, at least 20, at least 25, at least 30, at least 35, at least 40, at least 45, at least 50, at least 55, or at least 60 fewer, e.g., 15-60, 15-50, 15-40, 15-30, 15-20, 20-60, 20-50, 20-40, 20-30, 30-60, 30-50, 30-40, 40-60, 40-50, or 50-60 fewer, functional recognition sites (e.g., functional naturally occurring recognition sites) of two or more (e.g., 2-5, 2, 3, 4, or 5) selected type IIS restriction enzymes relative to a nucleotide sequence in a reference baculovirus genome, e.g., a nucleotide sequence in a wild-type baculovirus genome.
   E754. The method of any one of embodiments E748-E753, wherein the variant baculovirus genome comprises:
(i) no functional recognition sites (e.g., functional naturally occurring recognition sites) of two or more (e.g., 2, 3, 4, or 5) selected type II restriction enzymes;
(ii) no functional recognition sites (e.g., functional naturally occurring recognition sites) of a selected type of type II restriction enzymes; or
(iii) no functional recognition sites (e.g., functional naturally occurring recognition sites) for type II restriction enzymes.
   E755. The method of any one of embodiments E748-E754, wherein the variant baculovirus genome comprises no functional recognition sites (e.g., functional naturally occurring recognition sites) of a selected type IIS restriction enzyme.
   E756. The method of any one of embodiments E748-E755, wherein the variant baculovirus genome comprises no functional recognition sites (e.g., functional naturally occurring recognition sites) of two or more (e.g., 2, 3, 4, or 5) selected type IIS restriction enzymes.
   E757. The method of any one of embodiments E748-E756, wherein the variant baculovirus genome comprises no functional recognition sites (e.g., functional naturally occurring recognition sites) for type IIS restriction enzymes.
   E758. The method of any one of embodiments E748-E757, wherein the variant baculovirus genome is devoid of:
(i) recognition sites (e.g., functional naturally occurring recognition sites) of a selected type II restriction enzyme
(ii) recognition sites (e.g., functional naturally occurring recognition sites) for 1-5 (e.g., 1-4, 1-3, 1-2, 1, 2, 3, 4, or 5) selected type II restriction enzymes; or
(iii) recognition sites (e.g., functional naturally occurring recognition sites) for type II restriction enzymes.
   E759. The method of any one of embodiments E748-E758, wherein the variant baculovirus genome is devoid of type IIS restriction enzyme sites.
   E760. The method of any one of embodiments E748-E759, wherein variant baculovirus genome is devoid of recognition sites for a selected type IIS restriction enzyme.
   E761. The method of any one of embodiments E748-E760, wherein variant baculovirus genome is devoid of recognition sites for two or more (e.g., 2, 3, 4, or 5) selected type IIS restriction enzymes.
   E762. The plurality of fragments of any one of embodiments E748-E761, wherein the variant baculovirus nucleotide sequence is devoid of type IIS restriction enzyme sites.
   E763. The method of any one of embodiments E748-E762, wherein the restriction enzyme site is recognized by a restriction enzyme having one, two, three, four or all of the following properties:
(i) is capable of producing a 5′ overhang upon cleavage by the restriction enzyme;
(ii) cleavage by the restriction enzyme results in at least one or at least two (e.g., one or two) base pairs outside the 5′ overhang in its recognition site;
(iii) is capable of producing a 3′ overhang upon cleavage by the restriction enzyme; (iv) is heat inactivatable; and/or
(v) recognizes a stretch of at least 4-8 base pairs, e.g., at least 4 base pairs, at least 5 base pairs, at least 6 base pairs, at least 7 base pairs, or at least 8 base pairs (e.g., 8 base pairs).
   E764. The method of any one of embodiments E748-E763, wherein the restriction enzyme site is a type II restriction enzyme site.
   E765. The method of any one of embodiments E748-E764, wherein the restriction enzyme site is recognized by a restriction enzyme that is able to be used in aa Gibson Assembly™ cloning and ligation method (e.g., a method as described in Gibson et al. “Enzymatic assembly of DNA molecules up to several hundred kilobases,” Nat. Methods, 2009, 6(5):343-5; the contents of which are hereby incorporated by reference in their entirety), e.g., a Gibson Assembly™ compatible enzyme.
   E766. The method of any one of embodiments E748-E765, wherein the restriction enzyme site is recognized by a restriction enzyme selected from AcuI, AlwI, AaR1, BaeI, BbsI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, EarI, EciI, Esp3I, FauI, FokI, HgaI, HphI, HpyAV, MbolI, MlyI, MnlI, Nme-AIII, PaqCI, PleI, SapI, SfaNI, AbsI, Acc65I, AgeI, AgsI, ArsI, AscI, AsiGI, AsiSI, Asp718I, AspA2I, AsuNHI, AvrII, Alw44I, AxyI, BamHI, BelI, BcnI, BglII, Bpu1102I, BlnI, BmtI, BsaI, Bse21I, BshTI, BsmBI, Bso31I, BspOI, BspTNI, BstENI, Bsp1191, Bsp120I, Bsu15I, Bsu36I, CciNI, Cfr13I, Csp6I, Cfr9I, CspAI, Ec1136II, Eco31I, Eco53kI, Eco81I, Eco91I, Eco52I, EcoICRI, EcoNI, EcoRI, Esp3I, FseI, Hin6I, HindIII, HpaII, I-CeuI, I-PpoI, I-SceI, KpnI, Kpn2I, LmnI, LpnPI, MauBI, MspI, MluI, NcoI, NdeI, NheI, NotI, PalAI, PI-PspI, PI-SceI, PinAI, Psp124BI, RgaI, RigI, SacI, SalI, SbfI, SdaI, SfaAI, SgfI, SgsI, SmaI, SrfI, Sse8387I, SstI, TaqI, TspMI, XagI, XbaI, XhoI, XmaI, XmaCI, XmaJI, MunI, HinfI, BspTI, Pfl23II, VspI, Bsp1407I, Psp1406I, Tru1I, Bpu101, BcuI, PagI, BshTI, XmaJI, PfoI, SgsI, PteI, KflI, or an isoschizomer of any of the aforesaid restriction enzymes.
   E767. The method of any one of embodiments E748-E766, wherein the restriction enzyme site is a type IIS restriction enzyme site.
   E768. The method of any one of embodiments E748-E767, wherein the type IIS restriction enzyme site is recognized by a restriction enzyme selected from AcuI, AlwI, AaR1, BaeI, BbsI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, EarI, EciI, Esp3I, FauI, FokI, HgaI, HphI, HpyAV, MbolI, MlyI, MnlI, Nme-AIII, PaqCI, PleI, SapI, or SfaNI, or an isoschizomer of any of the aforesaid restriction enzymes.
   E769. The method of any one of embodiments E748-E768, wherein the type IIS restriction enzyme sites are a BsmBI restriction enzyme site, a BsaI restriction enzyme site, a PaqCI restriction enzyme site, or a combination thereof.
   E770. The method of any one of embodiments E748-E769, wherein the type IIS restriction enzyme site is a BsmBI restriction enzyme site.
   E771. The method of any one of embodiments E748-E770, wherein the restriction enzyme site is recognized by a restriction enzyme selected from AbsI, Acc65I, AgeI, AgsI, ArsI, AscI, AsiGI, AsiSI, Asp718I, AspA2I, AsuNHI, AvrII, Alw44I, AxyI, BamHI, BelI, BcnI, BglII, Bpu1102I, BlnI, BmtI, BsaI, Bse21I, BshTI, BsmBI, Bso31I, BspOI, BspTNI, BstENI, Bsp119I, Bsp120I, Bsu15I, Bsu36I, CciNI, Cfr13I, Csp6I, Cfr9I, CspAI, Ec1136II, Eco31I, Eco53kI, Eco81I, Eco91I, Eco52I, EcoICRI, EcoNI, EcoRI, Esp3I, FseI, Hin6I, HindIII, HpaII, I-CeuI, I-PpoI, I-SceI, KpnI, Kpn2I, LmnI, LpnPI, MauBI, MspI, MluI, NcoI, NdeI, NheI, NotI, PalAI, PI-PspI, PI-SceI, PinAI, Psp124BI, RgaI, RigI, SacI, SalI, SbfI, SdaI, SfaAI, SgfI, SgsI, SmaI, SrfI, Sse8387I, SstI, TaqI, TspMI, XagI, XbaI, XhoI, XmaI, XmaCI, XmaJI, MunI, HinfI, BspTI, Pfl23II, VspI, Bsp1407I, Psp1406I, Tru1I, Bpu10I, BcuI, PagI, BshTI, XmaJI, PfoI, SgsI, PteI, KflI, or an isoschizomer of any of the aforesaid restriction enzymes.
   E772. The method of any one of embodiments E748-E771, wherein the restriction enzyme site is recognized by AgeI, AvrII, BamHI, NheI, AscI, NotI, or an isoschizomer of any of the aforesaid restriction enzymes.
   E773. The method of any one of embodiments E748-E772, wherein the restriction enzyme site is recognized by a restriction enzyme selected from AgeI, AvrII, BamHI, NheI, or an isoschizomer of any of the aforesaid restriction enzymes.
   E774. The method of any one of embodiments E748-E772, wherein the restriction enzyme site is recognized by a restriction enzyme selected from AscI, NotI, or an isoschizomer of any of the aforesaid restriction enzymes.
   E775. The method of any one of embodiments E748-E774, wherein the at least one, two, three, four or more of the at least 5 fewer functional type IIS restriction enzyme sites are different type IIS restriction enzyme sites, e.g., a first type IIS restriction enzyme site and a second type IIS restriction enzyme site, optionally wherein:
(i) the first type IIS restriction enzyme site is recognized by a restriction enzyme selected from AcuI, AlwI, AaR1, BaeI, BbsI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, EarI, EciI, Esp3I, FauI, FokI, HgaI, HphI, HpyAV, MbolI, MlyI, MnlI, Nme-AIII, PaqCI, PleI, SapI, or SfaNI, or an isoschizomer of any of the preceding restriction enzymes; and
(ii) the second type IIS restriction enzyme site is recognized by a restriction enzyme selected from AcuI, AlwI, AaR1, BaeI, BbsI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, EarI, EciI, Esp3I, FauI, FokI, HgaI, HphI, HpyAV, MbolI, MlyI, MnlI, Nme-AIII, PaqCI, PleI, SapI, or SfaNI, or an isoschizomer of any of the preceding restriction enzymes.
   E776. The method of any one of embodiments E748-E775, wherein each fragment comprises a different 5′ overhang and/or a 3′ overhang relative to the other fragments of the plurality.
   E777. The method of any one of embodiments E748-E776, wherein the unique 5′ overhang of one subgenomic fragment is partially complementary or fully complementary to the unique 3′ overhang of another subgenomic fragment.
   E778. The method of any one of embodiments E748-E777, wherein each subgenomic fragment of the plurality comprises a nucleotide sequence at the 5′ end that overlaps with the nucleotide sequence at the 3′ end of another subgenomic fragment of the plurality, wherein the region of overlap is unique to a pair of subgenomic fragments.
   E779. The method of any one of embodiments E748-E778, wherein the subgenomic fragments of the plurality are capable of ordered assembly based on the overlap in nucleotide sequence at the 5′ end of one subgenomic fragment with the nucleotide sequence at the 3′ end of another subgenomic fragment to generate a variant baculovirus genome or the baculovirus expression construct.
   E780. The method of embodiment E778 or E779, wherein the overlap in nucleotide sequence between the subgenomic fragments is 4-50 (e.g., 4-45, 4-40, 4-35, 4-30, 4-25, 4-20, 4-15, 4-10, 10-50, 10-45, 10-40, 10-35, 10-30, 10-25, 10-20, 10-15, 15-50, 15-45, 15-40, 15-35, 15-30, 15-25, 15-20, 20-50, 20-45, 20-40, 20-35, 20-30, 20-25, 25-50, 25-45, 25-40, 25-35, 25-30, 30-50, 30-45, 30-40, 30-35, 35-50, 35-45, 35-40, 40-50, 40-45) contiguous base pairs.
   E781. The method of any one of embodiments E748-E780, wherein the unique 5′ overhang and unique 3′ overhang in a subgenomic fragment result from cleavage of the fragment by a type IIS restriction enzyme.
   E782. The method of any one of embodiments E748-E781, wherein the 5′ and 3′ overhang each independently comprise 1-6 nucleotides in length, e.g., 2-5 nucleotides (e.g., 4 nucleotides).
   E783. The method of any one of embodiments E748-E782, wherein the unique 5′ overhang and unique 3′ overhang of the fragments are each independently selected from: ACAA, GGTC, GACC, CCAG, CTGG, CCTT, AAGG, TCAT, ATGA, TCGC, GCGA, AGAG, CTCT, AACT, AGTT, CGGT, ACCG, ATAC, GTAT, GAGT, ACTC, TTCC, GGAA, ATTA, TAAT, TCCT, AGGA, TCTA, TAGA, TGTA, TACA, GATG, CATC, or TTGT, wherein the sequences of the unique 5′ overhang and 3′ overhang in a fragment are different.
   E784. The method of any one of embodiments E748-E783, wherein the type IIS restriction enzyme site is a BsaI restriction enzyme site or a BsmBI restriction enzyme site.
   E785. The method of any one of embodiments E748-E784, wherein the assembled subgenomic fragments are covalently linked, e.g., using a ligase (e.g., a T4 ligase), for example using Golden Gate Assembly or Gibson Assembly™, such that a variant baculovirus genome is formed, optionally wherein the subgenomic fragments are ligated in a single step to generate a variant baculovirus genome.
   E786. The method of any one of embodiments E748-E785, wherein the reference baculovirus genome is partitioned into 2-20 subgenomic fragments (e.g., 2, 3, 4, 5, 6, 7, 8, 9, 10, 11, 12, 13, 14, 15, 16, 17, 18, 19, or 20 subgenomic fragments).
   E787. The method of any one of embodiments E748-E786, wherein each subgenomic fragment is about 100-25000 bp in length (e.g., about 100-1000 bp, about 100-10000, about 100-20000, about 100-25000, about 1000-10000 bp, about 1000-8000 bp, about 1000-5000 bp, about 1000-2500 bp, about 2500-25000 bp, about 2500-20000 bp, about 2500-15000 bp, about 2500-10000 bp, about 2500-5000 bp, about 5000-25000 bp, about 5000-20000 bp, about 5000-15000 bp, about 5000-10000 bp, about 7500-25000 bp, about 7500-20000, about 7500-15000 bp, about 7500-10000 bp, about 10000-25000 bp, about 10000-20000 bp, about 10000-15000 bp, about 15000-25000 bp, about 15000-20000 bp, about 20000-25000 bp, about 7000-9000 bp, or about 8000 bp in length) 100-10000 bp (e.g., about 100-1000 bp, about 1000-10000 bp, about 5000-10000 bp, or about 7000-9000 bp in length).
   E788. The method of any one of embodiments E748-E787, wherein one or more of the subgenomic fragments are present in a first carrier vector.
   E789. The method of any one of embodiments E788, wherein each of the one or more of the subgenomic fragments are present in separate first carrier vectors.
   E790. The method of any one of embodiments E748-E789, wherein each subgenomic fragment is further partitioned into 2-20 subfragments (e.g., 2, 3, 4, 5, 6, 7, 8, 9, 10, 11, 12, 13, 14, 15, 16, 17, 18, 19, or 20 subfragments), wherein each subfragment has a unique 5′ overhang and a unique 3′ overhang, or each subfragment has a nucleotide sequence at the 5′ end of one subfragment that overlaps with (e.g., is homologous with) the nucleotide sequence at the 3′ end of another subfragment, wherein the nucleotide sequence in the region of overlap is unique to a pair of fragments.
   E791. The method of embodiment E790, wherein each subfragment is about 50-1000 bp, e.g., about 50-900, 50-800, 50-700, 50-600, 50-500, 50-400, 50-300, 50-200, 50-100, 100-900, 100-800, 100-700, 100-600, 100-500, 100-400, 100-300, 100-200, 200-1000, 200-900, 200-800, 200-700, 200-600, 200-500, 200-400, 200-300, 400-1000, 400-900, 400-800, 400-700, 400-600, 400-500, 600-1000, 600-900, 600-800, 600-700, 800-1000, 800-900, 900-1000 bp (e.g., 100-800 bp, about 250-750 bp, about 400-600 bp, or about 500 bp) in length.
   E792. The method of embodiment E790 or E791, wherein the unique 5′ overhang of a first subfragment is complementary (e.g., partially complementary or fully complementary) to the unique 3′ overhang of the second subfragment, and/or the unique 3′ overhang of a first subfragment is complementary (e.g., partially complementary or fully complementary) to the unique 5′ overhang of the second subfragment.
   E793. The method of any one of embodiments E790-E792, wherein the unique 5′ overhang and the unique 3′ overhang of a subfragment result from cleavage by a restriction enzyme, e.g., a type II restriction enzyme, (e.g., a type IIS restriction enzyme).
   E794. The method of any one of embodiments E790-E793, wherein the restriction enzyme, e.g., a type II restriction enzyme, (e.g., a type IIS restriction enzyme), is different from the restriction enzyme used to generate the subgenomic fragments.
   E795. The method of any one of embodiments E790-E794, wherein the restriction enzyme has one, two, three, four, or all of the following properties:
(i) is capable of producing a 5′ overhang upon cleavage by the restriction enzyme;
(ii) cleavage by the restriction enzyme results in at least one or at least two (e.g., one or two) base pairs outside the 5′ overhang in its recognition site;
(iii) is capable of producing a 3′ overhang upon cleavage by the restriction enzyme; (iv) is heat inactivatable;
(v) recognizes a stretch of at least 4-8 base pairs, e.g., at least 4 base pairs, at least 5 base pairs, at least 6 base pairs, at least 7 base pairs, or at least 8 base pairs (e.g., 8 base pairs).
   E796. The method of any one of embodiments E790-E795, wherein the restriction enzyme is a type II restriction enzyme.
   E797. The method of any one of embodiments E790-E796, wherein the restriction enzyme is a restriction enzyme that is able to be used in a Gibson Assembly™ cloning and ligation method (e.g., a method as described in Gibson et al. “Enzymatic assembly of DNA molecules up to several hundred kilobases,” Nat. Methods, 2009, 6(5):343-5; the contents of which are hereby incorporated by reference in their entirety), e.g., a Gibson Assembly™ compatible enzyme.
   E798. The method of any one of embodiments E790-E797, wherein the restriction enzyme is selected from AcuI, AlwI, AaR1, BaeI, BbsI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, EarI, EciI, Esp3I, FauI, FokI, HgaI, HphI, HpyAV, MbolI, MlyI, MnlI, Nme-AIII, PaqCI, PleI, SapI, SfaNI, AbsI, Acc65I, AgeI, AgsI, ArsI, AscI, AsiGI, AsiSI, Asp718I, AspA2I, AsuNHI, AvrII, Alw44I, AxyI, BamHI, BelI, BcnI, BglII, Bpu1102I, BlnI, BmtI, BsaI, Bse21I, BshTI, BsmBI, Bso31I, BspOI, BspTNI, BstENI, Bsp1191, Bsp120I, Bsu15I, Bsu36I, CciNI, Cfr13I, Csp6I, Cfr9I, CspAI, Ec1136II, Eco31I, Eco53kI, Eco81I, Eco91I, Eco52I, EcoICRI, EcoNI, EcoRI, Esp3I, FseI, Hin6I, HindIII, HpaII, I-CeuI, I-PpoI, I-SceI, KpnI, Kpn2I, LmnI, LpnPI, MauBI, MspI, MluI, NcoI, NdeI, NheI, NotI, PalAI, PI-PspI, PI-SceI, PinAI, Psp124BI, RgaI, RigI, SacI, SalI, SbfI, SdaI, SfaAI, SgfI, SgsI, SmaI, SrfI, Sse83871, SstI, TaqI, TspMI, XagI, XbaI, XhoI, XmaI, XmaCI, XmaJI, MunI, HinfI, BspTI, Pfl23II, VspI, Bsp1407I, Psp1406I, Tru1I, Bpu10I, BcuI, PagI, BshTI, XmaJI, PfoI, SgsI, PteI, KflI, or an isoschizomer of any of the aforesaid restriction enzymes E799. The method of any one of embodiments E790-E798, wherein the restriction enzyme is a type IIS restriction enzyme.
   E800. The method of any one of embodiments E790-E799, wherein the type IIS restriction enzyme is selected from AcuI, AlwI, AaR1, BaeI, BbsI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, EarI, EciI, Esp3I, FauI, FokI, HgaI, HphI, HpyAV, MbolI, MlyI, MnlI, Nme-AIII, PaqCI, PleI, SapI, or SfaNI, or an isoschizomer of any of the aforesaid restriction enzymes.
   E801. The method of any one of embodiments E790-E800, wherein the type IIS restriction enzyme is a BsaI restriction enzyme, a BsmBI restriction enzyme, a PaqCI restriction enzyme, or a combination thereof.
   E802. The method of any one of embodiments E790-E801, wherein the type IIS restriction enzyme is a BsaI restriction enzyme.
   E803. The method of any one of embodiments E790-E802, wherein the restriction enzyme is selected from AbsI, Acc65I, AgeI, AgsI, ArsI, AscI, AsiGI, AsiSI, Asp718I, AspA2I, AsuNHI, AvrII, Alw44I, AxyI, BamHI, BelI, BcnI, BglII, Bpu1102I, BlnI, BmtI, BsaI, Bse21I, BshTI, BsmBI, Bso31I, BspOI, BspTNI, BstENI, Bsp119I, Bsp120I, Bsu15I, Bsu36I, CciNI, Cfr13I, Csp6I, Cfr9I, CspAI, Ec1136II, Eco31I, Eco53kI, Eco81I, Eco91I, Eco52I, EcoICRI, EcoNI, EcoRI, Esp3I, FseI, Hin6I, HindIII, HpaII, I-CeuI, I-PpoI, I-SceI, KpnI, Kpn2I, LmnI, LpnPI, MauBI, MspI, MluI, NcoI, NdeI, NheI, NotI, PalAI, PI-PspI, PI-SceI, PinAI, Psp124BI, RgaI, RigI, SacI, SalI, SbfI, SdaI, SfaAI, SgfI, SgsI, SmaI, SrfI, Sse83871, SstI, TaqI, TspMI, XagI, XbaI, XhoI, XmaI, XmaCI, XmaJI, MunI, HinfI, BspTI, Pfl23II, VspI, Bsp1407I, Psp1406I, Tru1I, Bpu10I, BcuI, PagI, BshTI, XmaJI, PfoI, SgsI, PteI, KflI, or an isoschizomer of any of the aforesaid restriction enzymes.
   E804. The method of any one of embodiments E790-E803, wherein the restriction enzyme is AgeI, AvrII, BamHI, NheI, AscI, NotI, or an isoschizomer of any of the aforesaid restriction enzymes.
   E805. The method of any one of embodiments E790-E804, wherein the restriction enzyme is selected from AgeI, AvrII, BamHI, NheI, or an isoschizomer of any of the aforesaid restriction enzymes.
   E806. The method of any one of embodiments E790-E804, wherein the restriction enzyme is selected from AscI, NotI, or an isoschizomer of any of the aforesaid restriction enzymes.
   E807. The method of any of the preceding embodiments, wherein the restriction enzyme is not EcoRI or wherein the restriction enzyme site is not EcoRI.
   E808. The method of any one of embodiments E790-E807, wherein one or more subfragments are capable of ordered assembly based on the complementarity of the 5′ overhang in one subfragment with the 3′ overhang in another subfragment, or the 3′ overhang in one subfragment with the 5′ overhang in another subfragment, to generate the subgenomic fragments and/or the baculovirus expression construct.
   E809. The method of any one of embodiments E790-E808, wherein the one or more subfragments are capable of ordered assembly based on the overlap (e.g., homology) in nucleotide sequence at the 5′ end in one subfragment with the nucleotide sequence at the 3′ end of another subfragment to generate the subgenomic fragments and/or the baculovirus expression construct.
   E810. The method of any one of embodiments E790-E809, wherein the subfragments are capable of ordered assembly based on the overlap in nucleotide sequence at the 5′ end of one subfragment with the nucleotide sequence at the 3′ end of another subfragment to generate the subgenomic fragments and/or the baculovirus expression construct.
   E811. The method of embodiment E809 or E810, wherein the overlap in nucleotide sequence between the subfragments is 4-50 (e.g., 4-45, 4-40, 4-35, 4-30, 4-25, 4-20, 4-15, 4-10, 10-50, 10-45, 10-40, 10-35, 10-30, 10-25, 10-20, 10-15, 15-50, 15-45, 15-40, 15-35, 15-30, 15-25, 15-20, 20-50, 20-45, 20-40, 20-35, 20-30, 20-25, 25-50, 25-45, 25-40, 25-35, 25-30, 30-50, 30-45, 30-40, 30-35, 35-50, 35-45, 35-40, 40-50, 40-45) contiguous base pairs.
   E812. The method of any one of embodiments E809-E811, wherein a 5′ exonuclease creates a single-stranded region of complementarity corresponding to the overlapping sequence between subfragments, producing subfragments capable of annealing with each other.
   E813. The method of embodiment E812, wherein any gaps between the annealed subfragments are filled in by a DNA polymerase.
   E814. The method of any one of embodiments E790-E813, wherein one or more of the fragments (e.g., subfragments) of the plurality are present in a second carrier vector.
   E815. The method of any one of embodiments E808-E814, wherein each of one or more fragments (e.g., subfragments) are present in separate second carrier vectors.
   E816. The method of any one of embodiments E748-E815, wherein one or more subgenomic fragments or subfragments comprise a modification, e.g., an insertion, deletion, or substitution.
   E817. The method of any one of embodiments E786-E816, wherein one or more of the subgenomic fragments or subfragments comprise a modification, e.g., an insertion, deletion, or substitution.
   E818. The method of embodiment E816 or E817, wherein the modification, e.g., an insertion, deletion, substitution, or mutation (e.g., frame-shift mutation), is present in a non-essential gene (e.g., auxiliary and/or per os infectivity factor gene) or a regulatory region of a non-essential gene (e.g., promoter modification or insertion of heterologous DNA adjacent to non-essential gene).
   E819. The method of embodiment E818, wherein the non-essential gene is selected from one, two, three, or more (e.g., 1, 2, 3, 4, 5, 6, 7, 8, 9, 10, 11, 12, 13, 14, 15, 16, 17, 18, 19, 20, or 21) of egt, p74 (PIF0), p26, SOD, ChiA, v-cath, p10, polyhedrin, ctx, odv-e56, PIF1, PIF2, PIF3, PIF4, PIF5, Tn7, AcORF-91, AcORF-108, AcORF-52, v-ubi, or p94.
   E820. The method of embodiment E819, which comprises modification, e.g., an insertion, deletion, substitution, or mutation (e.g., frame-shift mutation) in:
(i) v-cath and egt;
(ii) v-cath, egt, and SOD;
(iii) chiA, v-cath, egt, p26, p10, and p74;
(iv) chiA, v-cath, egt, p26, p10, p74, and SOD; or
(v) chiA, v-cath, egt, p26, p10, p74, SOD, AcORF-91, and AcORF-108.
   E821. The method of any one of embodiments E816-E820, wherein the modification comprises a deletion of a chiA gene, a v-cath gene, a p26 gene, a p10 gene, and/or a p74 gene, or a portion thereof.
   E822. The method of any one of embodiments E816-E821, wherein the modification comprises an insertion of a heterologous sequence in the non-essential gene or adjacent region.
   E823. The method of any one of embodiments E816-E822, wherein the modification comprises one or more mutations in the non-essential gene or adjacent region, optionally wherein the non-essential genes are present near (e.g., downstream or upstream) of a homologous repeat region 5 (hr5).
   E824. The method of any one of embodiments E816-E823, wherein the modification, e.g., an insertion, deletion, substitution, or mutation (e.g., frame-shift mutation), results in inactivation of a non-essential gene (e.g., auxiliary and/or per os infectivity factor gene) or a regulatory region of a non-essential gene (e.g., promoter modification or insertion of heterologous DNA adjacent to non-essential gene).
   E825. The method of any one of embodiments E816-E824, wherein the modification is introduced by chemical synthesis or PCR, e.g., PCR-based site-directed mutagenesis.
   E826. The method of any one of embodiments E816-E825, wherein one or more of the subgenomic fragments, or one or more of the subfragments, comprise a heterologous nucleotide sequence.
   E827. The method of embodiment E826, wherein the heterologous nucleotide sequence comprises a sequence of interest (e.g., a nucleotide sequence encoding a Rep-coding region, one or more Rep proteins, a VP-coding region, one or more VP proteins, and/or a payload).
   E828. The method of embodiment E827, wherein the sequence of interest is a nucleotide sequence encoding a polypeptide of interest or a nucleotide sequence of interest.
   E829. The method of embodiment E827 or E828, wherein the sequence of interest is operably linked to a first promoter (e.g., a baculovirus early promoter or a baculovirus early-late promoter) and/or a second promoter (e.g., a baculovirus late promoter or a baculovirus very late promoter).
   E830. The method of any one of embodiments E827-E829, wherein the sequence of interest is operably linked to a first promoter (e.g., a baculovirus early promoter or a baculovirus early-late promoter) and a second promoter (e.g., a baculovirus late promoter or a baculovirus very late promoter).
   E831. The method of embodiment E829 or E830, wherein the first and/or second promoter is selected from a baculovirus promoter, a viral promoter, an insect viral promoter, a non-insect viral promoter, a vertebrate viral promoter, a chimeric promoter from one or more species including virus and non-virus elements, a synthetic promoter, or a variant thereof.
   E832. The method of any one of embodiments E829-E831, wherein the first and/or second promoter chosen from a polh promoter, a p10 promoter, a ctx promoter, a gp64 promoter, an IE promoter, an IE-1 promoter, a p6.9 promoter, a Dmhsp70 promoter, a Hsp70 promoter, a p5 promoter, a p19 promoter, a p35 promoter, a p40 promoter, or a variant, e.g., functional fragment, thereof.
   E833. The method of any one of embodiments E829-E832, wherein the first and/or second promoter is selected from an a baculovirus early promoter, baculovirus late promoter, baculovirus early-late promoter, or a baculovirus very late promoter.
   E834. The method of any one of embodiments E829-E833, wherein first and/or second promoter is a baculovirus early promoter, baculovirus late promoter, or baculovirus early-late promoter.
   E835. The method of any one of embodiments E829-E834, wherein first and/or second promoter is a baculovirus early-late promoter (e.g., a gp64 promoter).
   E836. The method of any one of embodiments E829-E835, wherein the first or second promoter is a baculovirus very late promoter (e.g., a polh promoter).
   E837. The method of any one of embodiments E829-E836, wherein:
(a) the first promoter is an baculovirus early-late promoter and the second promoter is a baculovirus very late promoter,
(b) the first promoter is a baculovirus very late promoter and the second promoter is a baculovirus early-late promoter,
(c) the first promoter is a baculovirus early promoter and the second promoter is a baculovirus early-late promoter,
(d) the first promoter is a baculovirus early-late promoter and the second promoter is a baculovirus early promoter,
(e) the first promoter is a baculovirus early promoter and the second promoter is a baculovirus late promoter,
(f) the first promoter is a baculovirus late promoter and the second promoter is a baculovirus early promoter,
(g) the first promoter is a baculovirus early-late promoter and the second promoter is a baculovirus late promoter,
(h) the first promoter is a baculovirus late promoter and the second promoter is a baculovirus early-late promoter,
(i) the first promoter is a baculovirus late promoter and the second promoter is a baculovirus very-late promoter,
(j) the first promoter is a baculovirus very-late promoter and the second promoter is a baculovirus late promoter,
(k) the first promoter is a baculovirus early promoter and the second promoter is a baculovirus very late promoter,
(l) the first promoter is a baculovirus very late promoter and the second promoter is a baculovirus early promoter,
(m) the first promoter is a baculovirus early promoter and the second promoter is a baculovirus early promoter,
(n) the first promoter is a baculovirus early-late promoter and the second promoter is a baculovirus early-late promoter, or
(o) the first promoter is a baculovirus late promoter and the second promoter is a baculovirus late promoter.
   E838. The method of any one of embodiments E829-E837, wherein the first promoter is a baculovirus early-late promoter (e.g., gp64 promoter) and the second promoter is a baculovirus very late promoter (e.g., polh promoter).
   E839. The method of any one of embodiments E829-E838, wherein the first promoter is a baculovirus early promoter and the second promoter is a baculovirus late promoter.
   E840. The method of any one of embodiments E829-E839, wherein
(a) the baculovirus early promoter is selected from: a lef3 promoter, a dbp promoter, a p35 promoter, an orf82 promoter, an get promoter, an orf81 promoter, an orf122 promoter, a pk-2 promoter, an orf55 promoter, an etl promoter, a hcf-1 promoter, an etm promoter, a lef-2 promoter, a lef-6 promoter, an orf84 promoter, an orf118 promoter, or an orf111 promoter,
(b) the baculovirus early-late promoter is selected from: a lef2 promoter, a orf13 promoter, a orf23 promoter, a pkip promoter, a v-fgf promoter, a pp31 promoter, an odv-e66 promoter, an orf74 promoter, an orf79 promoter, an orf82 promoter, a p15 promoter, a cg30 promoter, a helicase promoter, an he65 promoter, an orf114 promoter, a pk-2 promoter, a gp64 promoter, a gp16 promoter, an alk-exo promoter, a p35 promoter, a me53 promoter, or an ie0 promoter,
(c) the baculovirus late promoter is selected from: a ptpase promoter, an Ac-bro promoter, a ctx promoter, an orf5 promoter, an orf19 promoter, an orf20 promoter, an sod promoter, a HisP promoter, an orf34 promoter, a v-ubi promoter, an orf38 promoter, an orf43 promoter, an orf44 promoter, an orf56 promoter, an orf59 promoter, an orf60 promoter, or an fp-25k promoter, and/or
(d) the baculovirus very late promoter is selected from a p10 promoter or a polh promoter.
   E841. The method of any one of embodiments E829-E840, wherein the first and/or second promoter comprises a TATA box motif and/or a CAGT motif.
   E842. The method of any one of embodiments E829-E841, wherein the first and/or second promoter comprises a TAAG motif (e.g., an ATAAG nucleotide sequence).
   E843. The method of any one of embodiments E829-E842, wherein the first and/or second promoter comprises both a TATA box motif and a TAAG motif.

E844. The method of any one of embodiments E829-E843, wherein the first or second promoter comprises a binding site for VLF-1.

E845. The method of any one of embodiments E829-E844, wherein the first or second promoter is a gp64 promoter (e.g., an OpMNPV gp64 promoter).
E846. The method of any one of embodiments E829-E845, wherein the first or second promoter is a polh promoter (e.g., an OpMNPV polh promoter or an AcMNPV polh promoter).
E847. The method of any one of embodiments E829-E846, wherein the first promoter is a gp64 promoter and the second promoter is a polh promoter, or wherein the first promoter is a polh promoter and the second promoter is a gp64 promoter.
E848. The method of any one of embodiments E829-E847, wherein the Rep-coding region is operably linked to a first promoter which is a baculovirus early-late promoter and a second promoter which is baculovirus very late promoter, e.g., a gp64 promoter and a polh promoter, optionally, wherein the Rep-coding region is present downstream of a homologous repeat region hr5.
E849. The method of any one of embodiments E829-E848, wherein the first promoter is a gp64 promoter and the second promoter is a polh promoter.
E850. The method of any one of embodiments E829-E837 or E840-E846, wherein the first promoter and the second promoter are the same.
E851. The method of embodiment E829-E850, wherein the first promoter and the second promoter are different.
E852. The method of embodiment E850, the first promoter and the second promoter are each a polh promoter.
E853. The method of embodiment E832-E851, wherein the gp64 promoter comprises the nucleotide sequence of SEQ ID NO: 217; a nucleotide sequence at least 70%, 75%, 80%, 85%, 90%, 95%, 96%, 97%, 98%, or 99% identical thereto; a nucleotide sequence comprising at least one, two, three, four, five, six, or seven, but no more than ten different nucleotides relative to SEQ ID NO: 217; or a nucleotide sequence comprising at least one, two, three, four, five, six, or seven, but no more than ten modifications (e.g., substitutions) relative to SEQ ID NO: 217.
E854. The method of embodiment E832-E852, wherein the polh promoter comprises the nucleotide sequence of SEQ ID NO: 167 or 220; a nucleotide sequence at least 70%, 75%, 80%, 85%, 90%, 95%, 96%, 97%, 98%, or 99% identical thereto; a nucleotide sequence comprising at least one, two, three, four, five, six, or seven, but no more than ten different nucleotides relative to SEQ ID NO: 167 or 220; or a nucleotide sequence comprising at least one, two, three, four, five, six, or seven, but no more than ten modifications (e.g., substitutions) relative to SEQ ID NO: 167 or 220.
E855. The method of any one of embodiments E832-E851, wherein the first promoter and the second promoter comprises the nucleotide sequence of SEQ ID NO: 221; a nucleotide sequence at least 70%, 75%, 80%, 85%, 90%, 95%, 96%, 97%, 98%, or 99% identical thereto; a nucleotide sequence comprising at least one, two, three, four, five, six, or seven, but no more than ten different nucleotides relative to SEQ ID NO: 221; or a nucleotide sequence comprising at least one, two, three, four, five, six, or seven, but no more than ten modifications (e.g., substitutions) relative to SEQ ID NO: 221.
E856. The method of embodiment E827 or E828, wherein the sequence of interest encodes a therapeutic protein or functional variant thereof, an antibody or antibody fragment; an enzyme; a component of a gene editing system; an RNAi agent (e.g., a dsRNA, siRNA, shRNA, pre-miRNA, pri-miRNA, miRNA, stRNA, lncRNA, piRNA, or snoRNA); or a combination thereof.
E857. The method of any one of embodiments E748-E856, wherein one or more of the subgenomic fragments, or one or more of the subfragments, encodes one or more AAV proteins.
E858. The method of any one of embodiments E748-E857, wherein one or more of the subgenomic fragments, or one or more of the subfragments, encodes an AAV Rep protein, e.g., Rep40, Rep52, Rep68, Rep78, or a combination thereof.
E859. The method of any one of embodiments E748-E858, wherein one or more of the subgenomic fragments, or one or more of the subfragments, encodes a Rep78 protein and/or a Rep52 protein.
E860. The method of any one of embodiments E748-E859, wherein one or more of the subgenomic fragments, or one or more of the subfragments, encodes an AAV capsid protein, e.g., a VP1 protein, a VP2 protein, a VP3, protein or a combination thereof.
E861. The method of any one of embodiments E748-E860, wherein one or more of the subgenomic fragments, or one or more of the subfragments, encodes an AAV1 capsid protein, an AAV2 capsid protein, an AAV3 capsid protein, an AAV4 capsid protein, an AAV5 capsid protein, an AAV6 capsid protein, an AAV8 capsid protein, an AAV9 capsid protein, an AAVrh10 capsid protein, or a variant thereof.
E862. The method of any one of embodiments E748-E861, wherein one or more of the subgenomic fragments, or one or more of the subfragments, encodes an AAV5 capsid protein or variant thereof, or an AAV9 capsid protein or variant thereof.
E863. The method of any one of embodiments E748-E862, wherein one or more of the subgenomic fragments, or one or more of the subfragments, encodes a payload.
E864. The method of embodiment E863, wherein the encoded payload is selected from a therapeutic protein or functional variant thereof; an antibody or antibody fragment; an enzyme; a component of a gene editing system; an RNAi agent (e.g., a dsRNA, siRNA, shRNA, pre-miRNA, pri-miRNA, miRNA, stRNA, lncRNA, piRNA, or snoRNA); or a combination thereof.
E865. The method of any one of embodiments E748-E864, wherein one or more of the subgenomic fragments, or one or more of the subfragments, are chemically synthesized or are non-templated fragments.
E866. A plurality of subgenomic fragments produced according to the method of any one of embodiments E748-E865.
E867. A method of producing a variant baculovirus genome comprising:
(i) providing the plurality of subgenomic fragments of embodiment E866,
(ii) assembling the plurality of subgenomic fragments into a variant baculovirus genome, and
(iii) optionally inserting the variant baculovirus genome into a baculovirus expression construct.
   E868. A variant baculovirus genome produced according to the method of embodiment E867.
   E869. A baculovirus expression construct comprising the variant baculovirus genome of embodiment E868.
   E870. A composition comprising the variant baculovirus genome of embodiment E868 or baculovirus expression construct of embodiment E869, and a carrier.
   E871. A kit for preparing a variant viral genome comprising the plurality of subgenomic fragments of embodiment E866, and instructions for use.
   E872. A baculovirus produced using the baculovirus expression construct of embodiment E869.
   E873. A composition comprising the baculovirus expression construct of any one of embodiments E1-E180 or E607-E611, and a carrier.
   E874. A method of modifying a variant baculovirus genome comprising:
(i) providing a plurality of subgenomic fragments of embodiment E866,
(ii) identifying one or more locations in the baculovirus genome to which one or more modifications (e.g., substitutions, insertions, or deletions), are desired,
(iii) selecting the corresponding subgenomic fragment that contains the one or more loci to which one or more modifications are to be introduced,
(iv) introducing the one or more modifications into the subgenomic fragment, thereby generating one or more modified subgenomic fragments;
(v) incubating the plurality of fragments under conditions suitable to form a variant baculovirus genome by ordered assembly of the plurality of subgenomic fragments, wherein the one or more modified subgenomic fragments replace the non-modified version of the one or more subgenomic fragments within the plurality,
thereby obtaining a modified variant baculovirus genome.
   E875. The method of embodiment E874, wherein one or more of subgenomic fragments is modified to introduce a heterologous nucleotide sequence from the variant baculovirus nucleotide sequence.
   E876. The method of embodiment E875, wherein the heterologous nucleotide sequence comprises a sequence of interest.
   E877. The method of embodiment E876, wherein the sequence of interest is a nucleotide sequence encoding a polypeptide of interest or a nucleotide sequence of interest.
   E878. The method of embodiment E876 or E877, wherein the sequence of interest encodes a therapeutic protein or functional variant thereof, an antibody or antibody fragment; an enzyme; a component of a gene editing system; an RNAi agent (e.g., a dsRNA, siRNA, shRNA, pre-miRNA, pri-miRNA, miRNA, stRNA, lncRNA, piRNA, or snoRNA); or a combination thereof.
   E879. The method of any one of embodiments E876-E878, wherein the heterologous nucleotide sequence, e.g., the sequence of interest, comprises one or more adeno-associated viral (AAV) gene encoding regions and/or a payload.
   E880. The method of any one of embodiments E876-E879, wherein the heterologous nucleotide sequence is chemically synthesized, a non-templated nucleotide sequence (e.g., non-templated fragment), or generated by a PCR based assay.
   E881. The method of any one of embodiments E876-E880, wherein the one or more modifications (e.g., an insertion, deletion, substitution, or mutation (e.g., frame-shift mutation)) is present in a non-essential gene (e.g., auxiliary and/or per os infectivity factor gene) or a regulatory region of a non-essential gene (e.g., promoter modification or insertion of heterologous DNA adjacent to non-essential gene).
   E882. The method of embodiment E881, wherein the non-essential gene is selected from one, two, three, or more (e.g., 1, 2, 3, 4, 5, 6, 7, 8, 9, 10, 11, 12, 13, 14, 15, 16, 17, 18, 19, 20, or 21) of egt, p74 (PIF0), p26, SOD, ChiA, v-cath, p10, polyhedrin, ctx, odv-e56, PIF1, PIF2, PIF3, PIF4, PIF5, Tn7, AcORF-91, AcORF-108, AcORF-52, v-ubi, or p94.
   E883. The method of embodiment E882, which comprises a modification, e.g., an insertion, deletion, substitution, or mutation (e.g., frame-shift mutation) in:
(i) v-cath and egt;
(ii) v-cath, egt, and SOD;
(iii) chiA, v-cath, egt, p26, p10, and p74;
(iv) chiA, v-cath, egt, p26, p10, p74, and SOD; or
(v) chiA, v-cath, egt, p26, p10, p74, SOD, AcORF-91, and AcORF-108.
   E884. The method of any one of embodiments E881-E883, wherein the modification comprises a deletion of a chiA gene, a v-cath gene, a p26 gene, a p10 gene, and/or a p74 gene, or a portion thereof.
   E885. The method of any one of embodiments E881-E884, wherein the modification comprises an insertion of a heterologous sequence in the non-essential gene or adjacent region.
   E886. The method of any one of embodiments E881-E885, wherein the modification comprises one or more mutations in the non-essential gene or adjacent region.
   E887. The method of any one of embodiments E881-E886, wherein the non-essential genes are present near (e.g., downstream or upstream) of a homologous repeat region 5 (hr5).
   E888. The method of any one of embodiments E875-E887, wherein the one or more modifications (e.g., an insertion, deletion, substitution, or mutation (e.g., frame-shift mutation)), results in inactivation of a non-essential gene (e.g., auxiliary and/or per os infectivity factor gene) or a regulatory region of a non-essential gene (e.g., promoter modification or insertion of heterologous DNA adjacent to non-essential gene).
   E889. The method of any one of embodiments E875-E888, wherein one or more of the subgenomic fragments comprises a disruption, e.g., a mutation (e.g., frameshift mutation), a deletion, an insertion, or inactivation, of a non-essential gene (e.g., auxiliary and/or per os infectivity factor gene) or a regulatory region of a non-essential gene (e.g., promoter modification or insertion of heterologous DNA adjacent to non-essential gene).
   E890. The method of any one of embodiments E875-E889, wherein the one or more modifications is introduced by chemical synthesis or PCR, e.g., PCR-based site-directed mutagenesis.
   E891. The method of any one of embodiments E875-E890, wherein one or more of the subgenomic fragments is modified to introduce a nucleotide sequence encoding an AAV Rep protein, e.g., Rep40, Rep52, Rep68, Rep78, or a combination thereof.
   E892. The method of any one of embodiments E875-E891, wherein one or more of the subgenomic fragments encodes a Rep78 protein and/or a Rep52 protein.
   E893. The method of any one of embodiments E875-E892, wherein one or more of the subgenomic fragments is modified to introduce a nucleotide sequence encoding an AAV capsid protein, e.g., a VP1 protein, a VP2 protein, a VP3, protein or a combination thereof.
   E894. The method of any one of embodiments E875-E893, wherein one or more of the subgenomic fragments is modified to introduce a nucleotide sequence encoding an AAV1 capsid protein, an AAV2 capsid protein, an AAV3 capsid protein, an AAV4 capsid protein, an AAV5 capsid protein, an AAV6 capsid protein, an AAV8 capsid protein, an AAV9 capsid protein, an AAVrh10 capsid protein, or a variant thereof.
   E895. The method of any one of embodiments E875-E894, wherein one or more of the subgenomic fragments is modified to introduce a nucleotide sequence encoding an AAV5 capsid protein or variant thereof, or an AAV9 capsid protein or variant thereof.
   E896. The method of any one of embodiments E875-E895, wherein one or more of the subgenomic fragments is modified to introduce a nucleotide sequence encoding a payload.
   E897. The method of embodiment E896, wherein the encoded payload is selected from a therapeutic protein or functional variant thereof; an antibody or antibody fragment; an enzyme; a component of a gene editing system; an RNAi agent (e.g., a dsRNA, siRNA, shRNA, pre-miRNA, pri-miRNA, miRNA, stRNA, lncRNA, piRNA, or snoRNA); or a combination thereof.
   E898. A variant baculovirus genome produced by the method of any one of embodiments E875-E890.
   E899. A variant baculovirus genome produced by the method of any one of embodiments E891-E895.
   E900. A variant baculovirus genome produced by the method of embodiment E896 or E897.
   E901. An AAV viral production system comprising the variant baculovirus genome comprising the AAV expression construct of embodiment E899 and a variant baculovirus genome comprising an AAV payload construct, optionally wherein the variant baculovirus genome is the variant baculovirus genome of embodiment E900.
   E902. The AAV viral production system of embodiment E901, which comprises an AAV viral production cell which comprises the AAV expression construct and AAV payload construct, optionally wherein the AAV viral production cell is an insect cell; optionally a Sf9 cell or a Sf21 cell.
   E903. A method of producing recombinant adeno-associated virus (rAAV) particle in an AAV viral production cell, the method comprising: (i) providing an AAV viral production system of embodiment E901 or E902, wherein the AAV expression construct comprises one or more VP-coding regions which comprise one or more nucleotide sequences encoding VP1, VP2 and VP3 capsid proteins; (ii) transfecting the AAV viral production system into an AAV viral production cell; (iii) exposing the AAV viral production cell to conditions which allow the AAV viral production cell to process the AAV expression construct and the AAV payload construct into rAAV particles; and, optionally, (iv) collecting the rAAV particles from the AAV viral production cell.
   E904. The method of embodiment E903, wherein the AAV viral production cell is an insect cell; optionally a Sf9 cell or a Sf21cell.
   E905. A recombinant adeno-associated virus (rAAV) particle produced by the method of embodiment E903 or E904.
   E906. A pharmaceutical composition comprising the rAAV particle of embodiment E905 and a pharmaceutically acceptable excipient.

### BRIEF DESCRIPTION OF DRAWINGS

The foregoing and other objects, features and advantages will be apparent from the following description of particular embodiments of the present disclosure, as illustrated in the accompanying figures.

FIG. 1 is a map showing the BsmBI and BsaI restriction enzyme recognition sites in the AcMNPV strain E2 baculovirus genome.

FIG. 2 depicts the strategy of comparing the sequence of the type II restriction site to be mutated with corresponding sites in other alpha-baculovirus genomes to determine substitutions which would minimize the possibility of impacting an essential baculovirus function.

FIG. 3 depicts the use of synthetic ‘B’ fragments or subgenomic ‘A’ fragments with 4-base overhangs to drive assembly of the fragments in a desired order and for compatibility with the Golden Gate Assembly approach.

FIG. 4 depicts the generation of 256 synthetic ‘B’ fragments (approximately 0.5 kb each), which form the 16 subgenomic ‘A’ fragments (approximately 8.0 kb each), which ultimately form the synthetic AcMNPV-E2 genome (approximately 133 kb).

FIG. 5 is a circular representation of the assembled ‘A’ fragments and ‘B’ fragments and their overhang sequences. The smaller font indicates the overhangs used to assemble the ‘A’ fragments from the ‘B’ fragments. The larger bolded font indicate the overhangs used to assemble the complete the genome from the ‘A’ fragments. The large bolded font overhang sequences for cloning into the bacmid are ACAA (shown) and CATC (not shown, but corresponding to the end overhang of the ‘A’ fragment that begins with the TACA overhang).

FIG. 6 shows the digestion patterns with the indicated restriction enzymes of the source bacmid having the AcMNPV strain E2 genome (“ctrl”) and a de novo-generated synthetic bacmid (“syn”).

FIG. 7 depicts the strategy of introducing modifications (e.g., substitutions) into a baculovirus genome using the Golden Gate Assembly strategy.

FIG. 8 shows that baculovirus prepared using the synthetic bacmid described in Example 1 is replication competent, but exhibited a replication defect, as demonstrated by the lower titers relative to baculovirus prepared using a reference bacmid comprising the wild-type AcMNPV-E2 genome with the exception of removal of the polyhedrin locus (“Control”).

FIGS. 9A and 9B show that baculovirus prepared using a synthetic bacmid with corrected p49 and lef8 genes (Synbac2) showed comparable replication with baculovirus produced from a reference bacmid comprising the wild-type AcMNPV-E2 genome with the exception of removal of the polyhedrin locus (“Control”) both after transfection of Sf9 cells (FIG. 9A) and after infection of Sf9 cells with an MOI TCID50 (tissue culture infective dose 50) of 3.

FIGS. 10A and 10B show that comparable expression of rAAV1 proteins and rAAV1 production were achieved with Sf9 cells co-infected with baculovirus generated from Synbac2 engineered to express Rep/AAV1 capsid proteins and Synbac2 engineered to express SEAP. As a control, Sf9 cells were co-infected with baculovirus generated from Expression Construct 1 (improved version of a bacmid comprising the wild-type AcMNPV-E2 genome which has multi-passage stability and achieves increased VP ratios and capsid quality relative to the Control bacmid from Example 2). Expression Construct 1 was engineered to express Rep/AAV1 capsid proteins, and Synbac2 was engineered to express SEAP. Viral titers (vg/mL) were determined by qPCR.

### DETAILED DESCRIPTION

### Overview

Baculovirus expression systems are a widely used tool in recombinant protein production. Their high scalability and productivity have been further extended to the production of recombinant adeno-associated virus (rAAV). However, baculovirus-based rAAV production is hindered by several factors including passage stability, complexity, and the number of protein products needed to support rAAV replication, and the generally low-throughput and bespoke nature of techniques used to modify large viral genomes.

The variant baculovirus genomes and methods of production described herein offer several advantages over current baculovirus expression vector systems by providing the unprecedented ability to synthesize large genomes (>130 kb) and/or modify large genomes at the single-nucleotide level, allowing for, e.g., the efficient design and production of streamlined baculovirus vectors for biologics production and development. The variant baculovirus genomes are formed in a modular manner based on a strategy which allows for efficient and rapid variant baculovirus vector development. By partitioning a reference baculovirus genome (e.g., an AcMNPV genome) into multiple subgenomic regions or fragments (or partitioning each subgenomic region or subfragment even further into multiple subregions or subfragments), modifications can be made to any locus within a large baculovirus genome. Upon introduction of the desired modifications (e.g., insertions, deletions, or substitutions), the various subgenomic fragments of the baculovirus genome, including the modified subgenomic fragment(s) which replace their wild-type counterparts, undergo ordered assembly based on the unique 5′ and 3′ overhangs in each subgenomic fragment to generate a variant baculovirus genome. The removal of recognition sites for one or more restriction enzymes which cut outside their recognition sites (e.g., type II restriction enzymes, such as type IIS restriction enzymes) throughout the genome allows for unique 5′ and 3′ overhangs which promote the assembly of subgenomic fragments in a desired order to be designed. As shown in the Examples, the variant baculovirus genomes showed no significant difference in baculovirus growth kinetics or rAAV production compared to their wild-type counterparts, suggesting that neither baculovirus replication nor very-late gene expression is compromised by the design or assembly method. Without wishing to be bound by theory, it is believed in some embodiments, that the compositions and methods described herein can enable de novo combination of several modules to generate a modified, variant baculovirus, without the constraints of previous baculovirus designs.

In some embodiments, the variant baculovirus genome is assembled using Golden Gate Assembly, e.g., using type IIS restriction enzymes such as but not limited to BsaI, BsmBI, AarI, PaqC1, and isoschizomers thereof, all of which are commercially available. The enzyme recognition sites for BsaI and BsmBI are 6 base pairs in length and the recognition site for PaqC1 is 7 base pairs in length. Without wishing to be bound by theory, Golden Gate Assembly allows for high-fidelity assembly of multiple fragments to generate the variant baculovirus genome.

In other embodiments, the variant baculovirus is assembled using Gibson Assembly™, e.g., using enzymes with recognition sites of 8 base pairs or longer, e.g., PacI, AscI, SrfI, PspXI, or AgeI.

### Viral Expression Constructs, Fragments, and Genomes

Provided herein are portions of entire viral genomes (e.g., recombinant variant viral genomes, such as variant baculovirus genomes), and constructs (e.g., expression constructs, such as bacmids) comprising the same, which can be produced by the ordered assembly of a plurality of fragments (e.g., subgenomic fragments and/or subfragments) which partially or fully constitute the nucleotide sequence of a reference viral genome (e.g., a wild-type baculovirus genome), as well as methods of producing, altering, and using the same. In some embodiments, the reference viral genome is not a coronavirus genome (e.g., a SARS coronavirus genome, such as a SARS-CoV-2 genome).

### Viral Expression Constructs

The viral expression constructs described herein may comprise two or more subgenomic regions comprising unique junctions and a variant viral nucleotide sequence (e.g., variant baculovirus nucleotide sequence) comprising fewer functional restriction enzyme sites (e.g., naturally occurring restriction enzyme sites), e.g., type II restriction enzyme sites (e.g., type IIS restriction sites), than a reference viral genome (e.g., reference baculovirus genome). In some embodiments, the viral expression construct is a baculovirus expression construct.

a. Subgenomic Regions and Subregions

The two or more subgenomic regions of a baculovirus expression construct may, for example, correspond to an entire baculovirus genome (e.g., a wild-type or variant baculovirus genome), or one or more portions thereof (e.g., a variant baculovirus genome with one or more modifications, such as the deletion of one or more non-essential genes, such as auxiliary and/or per os infectivity factor genes).

In some embodiments, the baculovirus expression construct comprises at least 3, 4, 5, 6, 7, 8, 9, 10, 11, 12, 13, 14, 15, 16, 17, 18, 19, 20, 21, 22, 23, 24, 25, 26, 27, 28, 29, or 30 subgenomic regions. In some embodiments, the baculovirus expression construct comprises 2, 3, 4, 5, 6, 7, 8, 9, 10, 11, 12, 13, 14, 15, 16, 17, 18, 19, 20, 21, 22, 23, 24, 25, 26, 27, 28, 29, 30, 2-30, 2-25, 2-20, 2-15, 2-10, 2-5, 5-30, 5-25, 5-20, 5-15, 5-10, 10-30, 10-25, 10-20, 10-15, 15-30, 15-25, 15-20, 20-30, 20-25, or 25-30 subgenomic regions. In some embodiments, the baculovirus expression construct comprises 10-20, 14-18, 15-17, or 16 subgenomic regions.

In some embodiments, a subgenomic region is about 100-25000 bp in length, for example, about 100-20000, 100-15000, 100-10000, 100-9000, 100-8000, 100-7000, 100-6000, 100-5000, 100-4000, 100-3000, 100-2000, 100-1000, 100-500, 100-300, 100-200, 100-150, 250-25000, 250-20000, 250-15000, 250-10000, 250-9000, 250-8000, 250-7000, 250-6000, 250-5000, 250-4000, 250-3000, 250-2000, 250-1000, 250-500, 500-25000, 500-20000, 500-15000, 500-10000, 500-9000, 500-8000, 500-7000, 500-6000, 500-5000, 500-4000, 500-3000, 500-2000, 500-1000, 750-25000, 750-20000, 750-15000, 750-10000, 750-9000, 750-8000, 750-7000, 750-6000, 750-5000, 750-4000, 750-3000, 750-2000, 750-1000, 1000-25000, 1000-20000, 1000-15000, 1000-10000, 1000-9000, 1000-8000, 1000-7000, 1000-6000, 1000-5000, 1000-4000, 1000-3000, 1000-2000, 2500-25000, 2500-20000, 2500-15000, 2500-10000, 2500-9000, 2500-8000, 2500-7000, 2500-6000, 2500-5000, 2500-4000, 2500-3000, 2500-2000, 5000-25000, 5000-20000, 5000-15000, 5000-10000, 5000-9000, 5000-8000, 5000-7000, 5000-6000, 6000-25000, 6000-20000, 6000-15000, 6000-10000, 6000-9000, 6000-8000, 6000-7000, 7000-25000, 7000-20000, 7000-15000, 7000-10000, 7000-9000, 7000-8000, 8000-25000, 8000-20000, 8000-15000, 8000-10000, 8000-9000, 9000-25000, 9000-20000, 9000-15000, 9000-10000, 10000-25000, 10000-20000, 10000-15000, 10000-12500, 12500-25000, 12500-20000, 12500-15000, 15000-25000, 15000-20000, 17500-25000, 17500-20000, 20000-25000, or 22500-25000 in length. In some embodiments, a subgenomic region is about 100, about 150, about 200, about 250, about 300, about 350, about 400, about 450, about 500, about 550, about 600, about 650, about 700, about 750, about 800, about 850, about 900, about 950, about 1000, about 1500, about 2000, about 2500, about 3000, about 3500, about 4000, about 4500, about 5000, about 5500, about 6000, about 6500, about 7000, about 7500, about 8000, about 8500, about 9000, about 9500, about 10000, about 12000, about 14000, about 16000, about 18000, about 20000, about 22000, about 24000, about 26000, about 28000, or about 30000 bp in length. In some embodiments, a subgenomic region is about 100-1000 bp, about 250-750 bp, about 500 bp, about 1000-10000 bp, about 5000-10000 bp, about 7000-9000 bp, or about 8000 bp in length. In some embodiments, a subgenomic region is about 500 bp in length. In some embodiments, a subgenomic region is about 8000 bp in length.

In some embodiments, a subgenomic region of the baculovirus expression construct corresponds to a subgenomic region of a reference baculovirus genome, or a variant version of the subgenomic region of a reference baculovirus genome, wherein the reference baculovirus genome has been partitioned (e.g., in silico) into a plurality of subgenomic regions (e.g., at least two subgenomic regions). In some embodiments, the reference baculovirus genome sequence comprises a plurality of subgenomic regions of approximately equal size. By way of example, in some embodiments, a reference baculovirus genome of 128 kilobases (kb) may be partitioned into 16 subgenomic regions of about 8000 bp each, and a subgenomic region of the baculovirus expression construct may have the nucleotide sequence of one of these 16 subgenomic regions, or a variant nucleotide sequence thereof. Accordingly, in some embodiments, one or more subgenomic regions of a baculovirus expression construct correspond to, or comprise, one or more subgenomic regions (or variant version(s) of the subgenomic regions) of a partitioned reference baculovirus genome.

The reference baculovirus genome sequence need not, however, be partitioned into subgenomic regions of about equal size, e.g., one or more of the subgenomic regions can be 5-80%, e.g., 5-60%, 5-30%, 10-80%, 10-60%, 10-30%, 30-80%, 30-60%, 50-80%, or 60-80%, smaller or larger than the remaining subgenomic regions, wherein the remaining subgenomic regions are of equal or about equal size.

In some embodiments, each subgenomic region of the baculovirus expression construct comprises two unique junction sites, wherein the first unique junction is present at the 5′ end of the subgenomic region, and the second unique junction is present at the 3′ end of the subgenomic region.

In some embodiments, the first unique junction and the second unique junction of a subgenomic region independently comprise 1-50, 4-50, 10-50, 20-50, 30-50, 40-50, 1-45, 4-45, 10-45, 15-45, 20-45, 30-45, 35-45, 40-45, 1-40, 4-40, 10-40, 20-40, 30-40, 1-35, 4-35, 10-35, 20-35, 30-35, 1-30, 4-30, 10-30, 20-30, 1-25, 4-25, 10-25, 15-25, 20-25, 1-20, 4-20, 10-20, 15-20, 1-15, 40-25, 10-15, 1-10, 2-10, 3-10, 4-10, 5-10, 6-10, 7-10, 8-10, 9-10, 1-8, 2-8, 3-8, 4-8, 5-8, 6-8, 7-8, 1-6, 2-6, 3-6, 4-6, 5-6, 1-5, 2-5, 3-5, 4-5, 1-4, 2-4, 3-4, 1-3, 2-3, 1, 2, 3, 4, 5, 6, 7, 8, 9, or 10 nucleotides in length, e.g., 2-5 nucleotides (e.g., 4 nucleotides). In some embodiments, the first unique junction and the second unique junction of a subgenomic region independently are 4 nucleotides in length. In some embodiments, the unique junctions represent a junction formed by complementarity between the 5′ overhang of one subgenomic fragment and the 3′ overhang of the immediately adjacent subgenomic fragment, or the 3′ overhang of one subgenomic fragment and the 5′ overhang of the immediately adjacent subgenomic fragment, during the ordered assembly of subgenomic fragments into a baculovirus nucleotide sequence (e.g., a baculovirus genome).

In some embodiments, the baculovirus expression construct comprises 2, 3, 4, 5, 6, 7, 8, 9, 10, 11, 12, 13, 14, 15, 16, 17, 18, 19, 20, 2-20, 5-20, 10-20, 15-20, 2-18, 5-18, 10-18, 14-18, 15-18, 2-16, 5-16, 10-16, 13-16, 14-16, 2-14, 5-14, 10-14, 2-12, 5-12, 10-12, 2-10, 5-10, or 2-5 unique junctions. In some embodiments, the baculovirus expression construct comprises 5-25, 10-20, 12-20, 14-18, 15-17, 15, 16, or 17 unique junctions. In some embodiments, the baculovirus expression construct comprises 17 unique junctions. In some embodiments, 2 of the 17 unique junctions are formed at the interface between the outermost subgenomic regions and the backbone of the baculovirus expression construct, e.g., a unique junction formed between the unique 5′ overhang of one subgenomic fragment with the unique 3′ overhang of the baculovirus expression construct backbone, and a unique junction formed between the unique 3′ overhang of another subgenomic fragment with the unique 5′ overhang of the baculovirus expression construct backbone.

In some embodiments, the baculovirus expression construct comprises unique junctions between subgenomic regions, for example, when generated using Gibson Assembly™ or Golden Gate Assembly which relies on the overlap of nucleotide sequence (e.g., a baculovirus genome sequence) at the 5′ end of one subgenomic fragment with the nucleotide sequence at the 3′ end of another subgenomic fragment (e.g., a baculovirus genome sequence) for ordered assembly, wherein the region of overlap is unique to a pair of subgenomic fragments.

In some embodiments, one or more subgenomic regions are partitioned into two or more subregions. In some embodiments, a subgenomic region is partitioned into 2, 3, 4, 5, 6, 7, 8, 9, 10, 11, 12, 13, 14, 15, 16, 17, 18, 19, 20, 21, 22, 23, 24, 25, 26, 27, 28, 29, 30, 1-30, 1-20, 1-10, 1-5, 1-4, 1-3, 1-2, 5-20, 10-20, 14-18, or 15-17 subregions (e.g., 16 subregions). In some embodiments, each subgenomic region of a baculovirus expression construct is partitioned into subregions, e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 2-30, 2-20, 2-10, 2-5, 2-4, 2-3, 5-20, 10-20, 14-18, or 15-17 subregions (e.g., 16 subregions).

In some embodiments, each subregion of a subgenomic region comprises two unique junction sites, wherein the first unique junction is present at the 5′ end of the subregion, and the second unique junction is present at the 3′ end of the subregion.

In some embodiments, the first unique junction and the second unique junction of a subregion independently comprise 1-10, 2-10, 3-10, 4-10, 5-10, 6-10, 7-10, 8-10, 9-10, 1-8, 2-8, 3-8, 4-8, 5-8, 6-8, 7-8, 1-6, 2-6, 3-6, 4-6, 5-6, 1-5, 2-5, 3-5, 4-5, 1-4, 2-4, 3-4, 1-3, 2-3, 1, 2, 3, 4, 5, 6, 7, 8, 9, or 10 nucleotides in length, e.g., 2-5 nucleotides (e.g., 4 nucleotides). In some embodiments, the first unique junction and the second unique junction of a subregion independently are 4 nucleotides in length. In some embodiments, the unique junctions represent a junction formed by complementarity between the 5′ overhang of one subregion and the 3′ overhang of the immediately adjacent subregion, or the 3′ overhang of one subregion and the 5′ overhang of the immediately adjacent subregion, during the ordered assembly of subregions into a subgenomic fragment.

In some embodiments, the subregions of a subgenomic region comprises unique junctions, for example, when a subgenomic region comprising subregions is generated using Gibson Assembly™ or Golden Gate Assembly. In such embodiments, the overlap of nucleotide sequence at the 5′ end of one subregion (e.g., a baculovirus genome sequence) with the nucleotide sequence at the 3′ end of another subregion (e.g., a baculovirus genome sequence) can promote the ordered assembly into a subgenomic region, wherein the region of overlap is unique to a pair of subfragments.

In some embodiments, a subgenomic region comprises 2, 3, 4, 5, 6, 7, 8, 9, 10, 11, 12, 13, 14, 15, 16, 17, 18, 19, 20, 2-20, 5-20, 10-20, 15-20, 2-18, 5-18, 10-18, 14-18, 15-18, 2-16, 5-16, 10-16, 13-16, 14-16, 2-14, 5-14, 10-14, 2-12, 5-12, 10-12, 2-10, 5-10, or 2-5 unique junctions. In some embodiments, a subgenomic region comprises 5-25, 10-20, 12-20, 14-18, 15-17, 15, 16, or 17 unique junctions. In some embodiments, a subgenomic region comprises 17 unique junctions. In some embodiments, 2 of the 17 unique junctions in a subgenomic region are formed at the interface between the outermost subregions of a subgenomic region and the 5′ overhang or 3′ overhang of the immediately adjacent subgenomic region or backbone of the baculovirus expression construct.

### B. Unique Overhangs and Junctions

In some embodiments, the unique 5′ overhang and the unique 3′ overhang of the fragments described herein (e.g., subgenomic fragments or subfragments) independently comprise 1, 2, 3, 4, 5, 6, 7, 8, 9, or 10 nucleotides. In some embodiments, the unique 5′ overhang and the unique 3′ overhang of the fragments (e.g., subgenomic fragments or subfragments) independently comprise 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4- 9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10 nucleotides. In some embodiments, the unique 5′ overhang and the unique 3′ overhang of the fragments comprise cohesive ends (e.g., staggered ends). In some embodiments, covalent attachment of the 5′ overhang of one fragment (e.g., subgenomic fragment or subfragment) to the 3′ overhang of another fragment (e.g., subgenomic fragment or subfragment) results in the formation of a unique junction.

A “unique junction” is formed by the covalent linkage of the unique 5′ overhang of one fragment with the unique 3′ overhang of another fragment. In some embodiments, the nucleotide sequence (e.g., 4 nucleotides) of the unique junction formed by two fragments is unique in that its sequence differs from that of any other junction formed by two fragments, or a fragment and the backbone of a destination vector (e.g., carrier vector, baculovirus expression construct, or BAC). In some embodiments, if multiple levels of fragments are used, e.g., if a genome has been partitioned into subgenomic fragments (one level), and the subgenomic fragments are partitioned further into subfragments (another level), then the unique junctions need be unique only within one level, provided that different restriction enzymes (e.g., type IIS restriction enzymes) are used to generate the unique 5′ and 3′ overhangs of fragments of the different levels.

In some embodiments, the unique 5′ overhang and the unique 3′ overhang result from cleavage of a fragment (e.g., subgenomic fragment or subfragment) by a restriction enzyme. In some embodiments, the restriction enzyme is a type II restriction enzyme. In some embodiments, the restriction enzyme that is able to be used in Gibson Assembly™ cloning and ligation method (e.g., a method as described in Gibson et al. “Enzymatic assembly of DNA molecules up to several hundred kilobases,” Nat. Methods, 2009, 6(5):343-5; the contents of which are hereby incorporated by reference in their entirety), e.g., a Gibson Assembly compatible enzyme. In some embodiments, the restriction enzyme is selected from AcuI, AlwI, AaR1, BaeI, BbsI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, EarI, EciI, Esp3I, FauI, FokI, HgaI, HphI, HpyAV, MbolI, MlyI, MnlI, Nme-AIII, PaqCI, PleI, SapI, SfaNI, AbsI, Acc65I, AgeI, AgsI, ArsI, AscI, AsiGI, AsiSI, Asp718I, AspA2I, AsuNHI, AvrII, Alw44I, AxyI, BamHI, BelI, BcnI, BglII, Bpu1102I, BlnI, BmtI, BsaI, Bse21I, BshTI, BsmBI, Bso31I, BspOI, BspTNI, BstENI, Bsp1191, Bsp120I, Bsu15I, Bsu36I, CciNI, Cfr13I, Csp6I, Cfr9I, CspAI, Ec1136II, Eco31I, Eco53kI, Eco81I, Eco91I, Eco52I, EcoICRI, EcoNI, EcoRI, Esp3I, FseI, Hin6I, HindIII, HpaII, I-CeuI, I-PpoI, I-SceI, KpnI, Kpn2I, LmnI, LpnPI, MauBI, MspI, MluI, NcoI, NdeI, NheI, NotI, PalAI, PI-PspI, PI-SceI, PinAI, Psp124BI, RgaI, RigI, SacI, SalI, SbfI, SdaI, SfaAI, SgfI, SgsI, SmaI, SrfI, Sse83871, SstI, TaqI, TspMI, XagI, XbaI, XhoI, XmaI, XmaCI, XmaJI, MunI, HinfI, BspTI, Pfl23II, VspI, Bsp1407I, Psp1406I, Tru1I, Bpu10I, BcuI, PagI, BshTI, XmaJI, PfoI, SgsI, PteI, KflI, or an isoschizomer of any of the aforesaid restriction enzymes. In some embodiments, the restriction enzyme is selected from AbsI, Acc65I, AgeI, AgsI, ArsI, AscI, AsiGI, AsiSI, Asp718I, AspA2I, AsuNHI, AvrII, Alw44I, AxyI, BamHI, BelI, BcnI, BglII, Bpu1102I, BlnI, BmtI, BsaI, Bse21I, BshTI, BsmBI, Bso31I, BspOI, BspTNI, BstENI, Bsp119I, Bsp120I, Bsu15I, Bsu36I, CciNI, Cfr13I, Csp6I, Cfr9I, CspAI, Ec1136II, Eco31I, Eco53kI, Eco81I, Eco91I, Eco52I, EcoICRI, EcoNI, EcoRI, Esp3I, FseI, Hin6I, HindIII, HpaII, I-CeuI, I-PpoI, I-SceI, KpnI, Kpn2I, LmnI, LpnPI, MauBI, MspI, MluI, NcoI, NdeI, NheI, NotI, PalAI, PI-PspI, PI-SceI, PinAI, Psp124BI, RgaI, RigI, SacI, SalI, SbfI, SdaI, SfaAI, SgfI, SgsI, SmaI, SrfI, Sse83871, SstI, TaqI, TspMI, XagI, XbaI, XhoI, XmaI, XmaCI, XmaJI, MunI, HinfI, BspTI, Pfl23II, VspI, Bsp1407I, Psp1406I, Tru1I, Bpu10I, BcuI, PagI, BshTI, XmaJI, PfoI, SgsI, PteI, KflI, or an isoschizomer of any of the aforesaid restriction enzymes. In some embodiments, the restriction enzyme is AgeI, AvrII, BamHI, NheI, AscI, NotI, or an isoschizomer of any of the aforesaid restriction enzymes. In some embodiments, the restriction enzyme is AgeI, AvrII, BamHI, NheI, or an isoschizomer of any of the aforesaid restriction enzymes. In some embodiments, the restriction enzyme is AscI, NotI, or an isoschizomer of any of the aforesaid restriction enzymes. In some embodiments, digestion by the restriction enzyme results in degenerative cohesive overhangs. In some embodiments, the restriction enzyme cleaves outside of its recognition site. In some embodiments, the restriction enzyme produces staggered ends (e.g., staggered ends of 2-4, 2, 3, or 4 bases). In some embodiments, the restriction enzyme recognizes asymmetric DNA sequences. In some embodiments, the restriction enzyme results in unique cohesive overhangs. In some embodiments, the restriction enzyme is a type IIS restriction enzyme. In some embodiments, the type IIS restriction enzyme is selected from, but not limited to, AcuI, AlwI, AaR1, BaeI, BbsI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, EarI, EciI, Esp3I, FauI, FokI, HgaI, HphI, HpyAV, MbolI, MlyI, MnlI, Nme-AIII, PaqCI, PleI, SapI, or SfaNI, or an isoschizomer of any of the aforesaid restriction enzymes. In some embodiments, the type IIS restriction enzyme is BsaI. In some embodiments, the type IIS restriction enzyme is BsmBI. Additional non-limiting restriction enzymes (e.g., type IIS restriction enzymes) suitable for use include, for example, AasI, Acc36I, AccB7I, AceIII, AcpII, Adel, AfiI, AjuI, AlfI, AloI, Alw26I, AlwNI, AlwXI, AmaCSI, ApaBI, ApyPI, AquII, AquIII, AquIV, ArsI, Asp10HII, BasI, Bbr7I, BbvI, BbvII, Bbv16II, Bce4I, Bce83I, BceCI, BcgI, Bco5I, Bco116I, BcoKI, BflI, BfuAI, BglI, Bli736I, Bme585I, BpiI, BplI, BpmI, BpuAI, BpuJI, BpuSI, BsAJI, Bsc4I, Bsc91I, Bsc107I, BscAI, Bse3DI, BseGI, BseKI, BseLI, BseMI, BseMII, BseXI, BseZI, BsgI, BsiYI, BslI, BslFI, Bso31I, BsoMAI, Bsp24I, Bsp423I, BspBS31I, BspCNI, BspD6I, BsplS4I, BspKT5I, BspLU11III, BspMI, BspST5I, BspTNI, BspTS514I, BspWI, Bst6I, Bst12I, Bst19I, Bst71I, BstAPI, BstBS32I, BstFZ438I, BstGZ53I, BstHZ55I, BstlZ316I, BstMAI, BstMWI, BstOZ616I, BstPZ418I, BstTS5I, BstV1I, BstV2I, BstXI, CaiI, CjeI, CjePI, CspCI, CstMI, DraIII, DrdI, DseDI, Eam1104I, Eco31I, Eco5TI, EcoA4I, Eco57MI, EcoO44I, EcoP15I, Esp3I, Esp1396I, GsuI, HaeIV, Hin4I, Hpy178III, Hpy188III, HpyF10VI, LweI, MmeI, MwoI, PfiBI, PfiMI, PhaI, PpiI, PsrI, RleAI, SdiI, SfiI, SmuI, Sth132I, StsI, TaqII, TspDTI, Tsp8EI, TspGWI, Tthl11II, Van91I, or VpaK11AI, or an isoschizomer of any of the aforesaid restriction enzymes. In some embodiments, the restriction enzyme recognizes a discontinuous site, for example, AasI, AccB7I, AcpII, Adel, AfiI, AjuI, AlwNI, ApaBI, ArsI, Asp10HII, BaeI, BasI, Bce4I, BceCI, BcgI, BfiI, BglI, Bsc4I, Bsc107I, BseLI, BsiYI, BslI, BspWI, BstAPI, BstH255I, BstlZ316I, BstMWI, BstXI, CaiI, BraIII, DrdI, DseDI, HpyF10VI, PflBI, PflMI, SdiI, SfiI, Tsp8EI, or Van91I, or an isoschizomer of any of the aforesaid restriction enzymes. In some embodiments, the restriction enzyme is capable of producing a 5′ overhang upon cleavage by the restriction enzyme. In some embodiments, cleavage by the restriction enzyme results in at least one or at least two (e.g., one or two) base pairs outside the 5′ overhang in its recognition site. In some embodiments, the restriction enzyme is capable of producing a 3′ overhang upon cleavage by the restriction enzyme. In some embodiments, the restriction enzyme is heat inactivatable. In some embodiments, the restriction enzyme recognizes a stretch of 4-8 base pairs, e.g., 4 base pairs, 5 base pairs, 6 base pairs, 7 base pairs, or 8 base pairs. In some embodiments, the restriction enzyme is not EcoRI.

In some embodiments, the unique 5′ overhang and the unique 3′ overhang are 4 nucleotides in length (e.g., overhangs generated by digestion with a type IIS restriction enzyme such as BsaI or BsmBI). In some embodiments, any combination of 4 nucleotides can be used as unique 5′ or unique 3′ overhangs, so long as the 4 nucleotides do not introduce a new recognition site for the restriction enzyme (e.g., type IIS restriction enzyme) used to generate the unique 5′ and 3′ overhangs. In some embodiments, the unique 5′ overhang and unique 3′ overhang of a fragment described herein (e.g., a subgenomic fragment or subfragment) are independently selected from AAAA, AAAC, AAAG, AAAT, AACA, AACC, AACG, AACT, AAGA, AAGC, AAGG, AAGT, AATA, AATC, AATG, AATT, ACAA, ACAC, ACAG, ACAT, ACCA, ACCC, ACCG, ACCT, ACGA, ACGC, ACGG, ACGT, ACTA, ACTC, ACTG, ACTT, AGAA, AGAC, AGAG, AGAT, AGCA, AGCC, AGCG, AGCT, AGGA, AGGC, AGGG, AGGT, AGTA, AGTC, AGTG, AGTT, ATAA, ATAC, ATAG, ATAT, ATCA, ATCC, ATCG, ATCT, ATGA, ATGC, ATGG, ATGT, ATTA, ATTC, ATTG, ATTT, CAAA, CAAC, CAAG, CAAT, CACA, CACC, CACG, CACT, CAGA, CAGC, CAGG, CAGT, CATA, CATC, CATG, CATT, CCAA, CCAC, CCAG, CCAT, CCCA, CCCC, CCCG, CCCT, CCGA, CCGC, CCGG, CCGT, CCTA, CCTC, CCTG, CCTT, CGAA, CGAC, CGAG, CGAT, CGCA, CGCC, CGCG, CGCT, CGGA, CGGC, CGGG, CGGT, CGTA, CGTC, CGTG, CGTT, CTAA, CTAC, CTAG, CTAT, CTCA, CTCC, CTCG, CTCT, CTGA, CTGC, CTGG, CTGT, CTTA, CTTC, CTTG, CTTT, GAAA, GAAC, GAAG, GAAT, GACA, GACC, GACG, GACT, GAGA, GAGC, GAGG, GAGT, GATA, GATC, GATG, GATT, GCAA, GCAC, GCAG, GCAT, GCCA, GCCC, GCCG, GCCT, GCGA, GCGC, GCGG, GCGT, GCTA, GCTC, GCTG, GCTT, GGAA, GGAC, GGAG, GGAT, GGCA, GGCC, GGCG, GGCT, GGGA, GGGC, GGGG, GGGT, GGTA, GGTC, GGTG, GGTT, GTAA, GTAC, GTAG, GTAT, GTCA, GTCC, GTCG, GTCT, GTGA, GTGC, GTGG, GTGT, GTTA, GTTC, GTTG, GTTT, TAAA, TAAC, TAAG, TAAT, TACA, TACC, TACG, TACT, TAGA, TAGC, TAGG, TAGT, TATA, TATC, TATG, TATT, TCAA, TCAC, TCAG, TCAT, TCCA, TCCC, TCCG, TCCT, TCGA, TCGC, TCGG, TCGT, TCTA, TCTC, TCTG, TCTT, TGAA, TGAC, TGAG, TGAT, TGCA, TGCC, TGCG, TGCT, TGGA, TGGC, TGGG, TGGT, TGTA, TGTC, TGTG, TGTT, TTAA, TTAC, TTAG, TTAT, TTCA, TTCC, TTCG, TTCT, TTGA, TTGC, TTGG, TTGT, TTTA, TTTC, TTTG, or TTTT, wherein the 5′ overhang and 3′ overhang in the fragment are not the same sequence. In some embodiments, the unique overhangs at the 5′ end and 3′ end of a fragment described herein (e.g., subgenomic fragment or fragment) are each independently selected from ACAA, GGTC, GACC, CCAG, CTGG, CCTT, AAGG, TCAT, ATGA, TCGC, GCGA, AGAG, CTCT, AACT, AGTT, CGGT, ACCG, ATAC, GTAT, GAGT, ACTC, TTCC, GGAA, ATTA, TAAT, TCCT, AGGA, TCTA, TAGA, TGTA, TACA, GATG, CATC, or TTGT, wherein the unique 5′ overhang and unique 3′ overhang in the fragment are not the same sequence.

In some embodiments, a baculovirus expression construct comprises two or more fragments (e.g., 2-50, 2-40, or 2-30 subfragments or 2-50, 2-40, or 2-30 subfragments) which are assembled in a desired order (e.g., to replicate the order of nucleotide sequences in a portion of a reference baculovirus genome or an entire reference baculovirus genome, or to alter the order of nucleotide sequences in a reference viral genome). In some embodiments, the desired order of assembly of fragments can be achieved by complementarity of the unique 3′ overhang of a first fragment with the unique 5′ overhang of a second fragment, wherein the nucleotide sequence of the unique 3′ overhang of the first fragment and the nucleotide sequence of the unique 5′ overhang of the second fragment are complementary to each other and form a first unique junction. In some embodiments, a third fragment comprises a unique 5′ overhang which is complementary to the unique 3′ overhang of the immediately preceding fragment (e.g., the second fragment) to form a second unique junction. In some embodiments, the unique 5′ overhang of the first fragment is complementary to the unique 3′ overhang of the baculovirus expression construct backbone and forms a unique junction, wherein, e.g., the baculovirus expression construct backbone has been digested with the same restriction enzyme used to generate the fragments. In some embodiments, the unique 3′ overhang of the last fragment (i.e., last in order of a plurality of fragments) is complementary to the unique 5′ overhang of the baculovirus expression construct backbone and forms a unique junction, wherein the baculovirus expression construct backbone has been digested with the same restriction enzyme used to generate the fragments. In some embodiments, the junction formed by a unique 5′ overhang of a subgenomic fragment and the 3′ overhang of another subgenomic fragment is a unique in sequence, with no other junction formed between two subgenomic fragments within the baculovirus expression construct having the same nucleotide sequence, thereby allowing for the ordered assembly of subgenomic fragments, e.g., into a baculovirus genome described herein. In some embodiments, the junction formed by a unique 5′ overhang of a subfragment and the 3′ overhang of another subfragment is a unique in sequence within a subgenomic fragment, with no other junction formed between two subfragments within the subgenomic fragment having the same nucleotide sequence, thereby allowing for the ordered assembly of subfragments into a subgenomic fragment.

By way of example, Table 1 provides a non-limiting list of unique 5′ and 3′ overhangs in each subgenomic fragment of a plurality of 16 subgenomic fragments which form a baculovirus genome, wherein the desired consecutive order of assembly begins from subgenomic fragment #1 and ends in subgenomic fragment #16. In some embodiments, a fragment has a unique 5′ overhang and a unique 3′ overhang selected from those listed in Table 1.

TABLE 1
Exemplary 4-base unique 5′ and 3′ overhang combinations
in a plurality of 16 subgenomic fragments

| | Unique 5′ overhang | Unique 3′ overhang |
|---|---|---|
| | (5′ to 3′) | (5′ to 3′) |
| Subgenomic fragment | | |
| 1 | ACAA¹ | GGTC |
| 2 | GACC | CCAG |
| 3 | CTGG | CCTT |
| 4 | AAGG | TCAT |
| 5 | ATGA | TCGC |
| 6 | GCGA | AGAG |
| 7 | CTCT | AACT |
| 8 | AGTT | CGGT |
| 9 | ACCG | ATAC |
| 10 | GTAT | GAGT |
| 11 | ACTC | TTCC |
| 12 | GGAA | ATTA |
| 13 | TAAT | TCCT |
| 14 | AGGA | TCTA |
| 15 | TAGA | TGTA |
| 16 | TACA | GATG² |
| Backbone | | |
| 17 | CATC | TTGT |

¹Forms a unique junction with the unique 3′ overhang of the baculovirus expression construct backbone digested with the same enzyme used to generate the subgenomic fragments
²Forms a unique junction with the unique 5′ overhang of the baculovirus expression construct backbone digested with the same enzyme used to generate the subgenomic fragments.

### C. Subgenomic Fragments

In some embodiments, the baculovirus expression vector comprises at least 2 subgenomic regions which are formed by at least a first subgenomic fragment and a second subgenomic fragment. In some embodiments, the subgenomic fragment is the same length as a subgenomic region. In some embodiments, each of the first subgenomic fragment and the second subgenomic fragment comprise a unique 5′ overhang and a unique 3′ overhang.

In some embodiments, the subgenomic fragments in a baculovirus expression construct reconstitute an entire baculovirus genome (e.g., a variant baculovirus genome). In some embodiments, the subgenomic fragments in a baculovirus expression construct reconstitute a portion of a baculovirus genome (e.g., a variant baculovirus genome with one or more modifications, such as the deletion of one or more non-essential genes, such as auxiliary and/or per os infectivity factor genes).

In some embodiments, the baculovirus expression vector comprises at least 2, for example, 3, 4, 5, 6, 7, 8, 9, 10, 11, 12, 13, 14, 15, 16, 17, 18, 19, 20, 21, 22, 23, 24, 25, 26, 27, 28, 29, 30, 2-30, 2-25, 2-20, 2-15, 2-10, 2-5, 5-30, 5-25, 5-20, 5-15, 5-10, 10-30, 10-25, 10-20, 10-15, 15-30, 15-25, 15-20, 20-30, 20-25, or 25-30 subgenomic fragments. In some embodiments, the baculovirus expression vector comprises 16 subfragments. In some embodiments, the baculovirus expression construct comprises 10-20, 14-18, 15-17, or 16 subgenomic fragments. In some embodiments, each of the 2-20 subgenomic fragments comprises a different 5′ overhang and/or a different 3′ overhang relative to the other subgenomic fragments.

In some embodiments, a subgenomic fragment is about 100-25000 bp in length, for example, about 100-20000, 100-15000, 100-10000, 100-9000, 100-8000, 100-7000, 100-6000, 100-5000, 100-4000, 100-3000, 100-2000, 100-1000, 100-500, 100-300, 100-200, 100-150, 250-25000, 250-20000, 250-15000, 250-10000, 250-9000, 250-8000, 250-7000, 250-6000, 250-5000, 250-4000, 250-3000, 250-2000, 250-1000, 250-500, 500-25000, 500-20000, 500-15000, 500-10000, 500-9000, 500-8000, 500-7000, 500-6000, 500-5000, 500-4000, 500-3000, 500-2000, 500-1000, 750-25000, 750-20000, 750-15000, 750-10000, 750-9000, 750-8000, 750-7000, 750-6000, 750-5000, 750-4000, 750-3000, 750-2000, 750-1000, 1000-25000, 1000-20000, 1000-15000, 1000-10000, 1000-9000, 1000-8000, 1000-7000, 1000-6000, 1000-5000, 1000-4000, 1000-3000, 1000-2000, 2500-25000, 2500-20000, 2500-15000, 2500-10000, 2500-9000, 2500-8000, 2500-7000, 2500-6000, 2500-5000, 2500-4000, 2500-3000, 2500-2000, 5000-25000, 5000-20000, 5000-15000, 5000-10000, 5000-9000, 5000-8000, 5000-7000, 5000-6000, 6000-25000, 6000-20000, 6000-15000, 6000-10000, 6000-9000, 6000-8000, 6000-7000, 7000-25000, 7000-20000, 7000-15000, 7000-10000, 7000-9000, 7000-8000, 8000-25000, 8000-20000, 8000-15000, 8000-10000, 8000-9000, 9000-25000, 9000-20000, 9000-15000, 9000-10000, 10000-25000, 10000-20000, 10000-15000, 10000-12500, 12500-25000, 12500-20000, 12500-15000, 15000-25000, 15000-20000, 17500-25000, 17500-20000, 20000-25000, or 22500-25000 in length. In some embodiments, a subgenomic fragment is about 100, about 150, about 200, about 250, about 300, about 350, about 400, about 450, about 500, about 550, about 600, about 650, about 700, about 750, about 800, about 850, about 900, about 950, about 1000, about 1500, about 2000, about 2500, about 3000, about 3500, about 4000, about 4500, about 5000, about 5500, about 6000, about 6500, about 7000, about 7500, about 8000, about 8500, about 9000, about 9500, about 10000, about 12000, about 14000, about 16000, about 18000, about 20000, about 22000, about 24000, about 26000, about 28000, or about 30000 bp in length. In some embodiments, a subgenomic fragment is about 100-1000 bp, about 250-750 bp, about 500 bp, about 1000-10000 bp, about 5000-10000 bp, about 7000-9000 bp, or about 8000 bp in length. In some embodiments, a subgenomic fragment is about 8000 bp in length.

In some embodiments, each of the 2-20 subgenomic fragments comprises a nucleotide sequence at the 5′ end that overlaps with (e.g., is homologous with) the nucleotide sequence at the 3′ end of another subgenomic fragment, wherein the region of overlap is unique to a pair of subgenomic fragments.

In some embodiments, each subgenomic fragment comprises a unique 5′ overhang and a unique 3′ overhang, e.g., a unique 5′ overhang and unique 3′ overhang described herein.

In some embodiments, the baculovirus expression construct comprises 10-20 (e.g., 12-18, 14-18, or 16) subgenomic fragments. In some embodiments, each of the 10-20 (e.g., 12-18, 14-18, or 16) subgenomic fragments comprise a unique 5′ overhang and a unique 3′ overhang.

In some embodiments, the unique 5′ overhang and/or the unique 3′ overhang of the subgenomic fragments independently comprise 4 nucleotides.

In some embodiments, a first unique junction of the at least two subgenomic regions in the baculovirus expression construct comprises the unique 5′ overhang of a first subgenomic fragment and the unique 3′ overhang of a second subgenomic fragment, wherein the unique 5′ overhang of the first subgenomic fragment is complementary to the unique 3′ overhang of the second subgenomic fragment. In some embodiments, a first unique junction of the at least two subgenomic regions in the baculovirus expression construct comprises the unique 3′ overhang of a first subgenomic fragment and the unique 5′ overhang of a second subgenomic fragment, wherein the unique 3′ overhang of the first subgenomic fragment is complementary to the unique 5′ overhang of the second subgenomic fragment. In some embodiments, the unique 5′ overhang of the first subgenomic fragment is partially complementary to the unique 3′ overhang of the second subgenomic fragment. In some embodiments, the unique 5′ overhang of the first subgenomic fragment is fully complementary to the unique 3′ overhang of the second subgenomic fragment. In some embodiments, the unique 3′ overhang of the first subgenomic fragment is partially complementary to the unique 5′ overhang of the second subgenomic fragment. In some embodiments, the unique 3′ overhang of the first subgenomic fragment is fully complementary to the unique 5′ overhang of the second subgenomic fragment.

In some embodiments, the unique 5′ overhang and unique 3′ overhang of the subgenomic fragments are generated by a restriction enzyme, e.g., a type IIS restriction enzyme, as described supra. In some embodiments, BsaI is used to generate the unique overhangs in the subgenomic fragments. In some embodiments, BsmBI is used to generate the unique overhangs in the subgenomic fragments.

In some embodiments, a subgenomic fragment comprises a nucleotide sequence at the 5′ end (e.g., a first baculovirus genome sequence) which overlaps with (e.g., is homologous with) the nucleotide sequence at the 3′ end of another subgenomic fragment (e.g., the first baculovirus genome sequence), resulting in a region of overlapping nucleotide sequences unique to a pair of subgenomic fragments. The nucleotide sequence at the 3′ end of the subgenomic fragment (e.g., a second baculovirus genome sequence) may overlap with the nucleotide sequence at the 5′ end of another different subgenomic fragment (e.g., the second baculovirus genome sequence), resulting in another region of overlapping nucleotide sequence unique to a pair of subgenomic fragments. These regions of overlapping nucleotide sequences allow for the ordered assembly of subgenomic fragments via, e.g., a Gibson Assembly™ reaction or a Golden Gate Assembly reaction, into a variant baculovirus genome or baculovirus expression construct.

In some embodiments, the subgenomic fragments are present in a carrier vector, e.g., a “first” carrier vector (as opposed to a “second” carrier vector comprising the subfragments described herein). In some embodiments, one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 2-30, 2-25, 2-20, 2-15, 2-10, 2-5, 5-30, 5-25, 5-20, 5-15, 5-10, 10-30, 10-25, 10-20, 10-15, 15-30, 15-25, 15-20, 20-30, 20-25, or 25-30) of the subgenomic fragments are present in a first carrier vector. In some embodiments, the first carrier vector comprises 10-20 (e.g., 12-18, 14-18, or 16) subgenomic fragments. In some embodiments, the first carrier vector comprises 16 subgenomic fragments.

In some embodiments, each of the subgenomic fragments are present in a separate carrier vector.

In some embodiments, the baculovirus expression vector comprises at least 2, for example, 3, 4, 5, 6, 7, 8, 9, 10, 11, 12, 13, 14, 15, 16, 17, 18, 19, 20, 21, 22, 23, 24, 25, 26, 27, 28, 29, 30, 2-30, 2-25, 2-20, 2-15, 2-10, 2-5, 5-30, 5-25, 5-20, 5-15, 5-10, 10-30, 10-25, 10-20, 10-15, 15-30, 15-25, 15-20, 20-30, 20-25, or 25-30 subgenomic regions which are formed by an equal number of subgenomic fragments, wherein each subgenomic fragment is present in a separate carrier vector. For example, in some embodiments, 2-20 (e.g., 5-20, 10-20, 12-18, 14-18, or 16) subgenomic fragments form the subgenomic regions, with each subgenomic fragment present in a separate carrier vector (e.g., for a total of 10-20 (e.g., 12-18, 14-18, or 16) carrier vectors). In some embodiments, the baculovirus expression construct comprises 16 subgenomic regions formed by 16 subgenomic fragments, with each of the 16 subgenomic fragments present in a separate carrier vector (i.e., total of 16 carrier vectors).

Any vector (e.g., commercially available vector) suitable for cloning and maintaining the subgenomic fragments are suitable for use as a carrier vector. In some embodiments, the backbone of the vector which is used as a carrier vector is devoid of recognitions sites for the restriction enzyme (e.g., type IIS restriction enzyme) used to generate the unique 5′ overhang and the unique 3′ overhang of the subgenomic fragments. In some embodiments, the backbone of the vector which is used as a carrier vector is devoid of recognitions sites for the restriction enzyme used to release the subgenomic fragments for, e.g., subsequent Gibson Assembly™ or Golden Gate Assembly. This, for example, avoids cutting within the vector and generating undesirable fragments when releasing the subgenomic fragment or subgenomic fragments from the carrier vector or separate carrier vectors with the restriction enzyme (e.g., type II restriction enzyme, e.g., type IIS restriction enzyme). In some embodiments, if the vector comprises recognition sites for the restriction enzyme used to generate the unique 5′ and 3′ overhangs of the subgenomic regions, then the vector may be altered (e.g., domesticated) to render the recognition site non-functional using conventional cloning techniques, e.g., site-directed mutagenesis.

In some embodiments, the carrier vector comprises a nucleotide sequence which confers resistance to an antibiotic or antibiotics. In some embodiments, the carrier vector comprises one or more (e.g., 1-3, 1-2, 1, 2, or 3) antibiotic resistance gene cassettes which confer resistance to one or more (e.g., 1-3, 1-2, 1, 2, or 3) antibiotics selected from kanamycin, ampicillin, spectinomycin, streptomycin, carbenecillin, bleomycin, erythromycin, polymyxin B, tetracycline, or chloramphenicol.

In some embodiments, the subgenomic fragments are generated from the first carrier vector or the separate carrier vectors using the same restriction enzyme (e.g., type II restriction enzyme, e.g., type IIS restriction enzyme). In some embodiments, the restriction enzyme is a type II restriction enzyme chosen from, but not limited to, Eco31I, BsaI Bso31I, BspTNI, Esp3I, BpiI, Alw26I, BsmAI, BstMAI, BmsI, BhaI, BscAI, BscUI, BsmNI, BspST5I, Bst19I, CjeP338II, LweI, Mva1312II, PhaI, SfaNI, LguI, BspQI, PsiSI, SapI, Alw44I, ApaLI, VneI, BamHI, BcnI, BglII, Bpu1102I, BlpI, Bsp1720I, CeIII, Bsp119I, AsuII, Bpu14I, BspT104I, BstBI, Csp45I, NspV, SfuI, Bsp120I, PspOMI, Bsu15I, BanIII, Bsa29I, BseCI, BshVI, BspDI, BspXI, BsuTUI, ClaI, Cfr10I, Bse118I, BsrFI, BssAI, Cfr13I, AspS9I, BmgT120I, PspPI, Sau96I, Csp6I, AfaI, CviQI, RsaNI, Eam1104I, Bst6I, EarI, EcoO109I, DraII, EcoRI, Eco47I, AvaII, Bme18I, SinI, VpaK11BI, Eco52I, BseX3I, BstZI, EagI, EcIXI, Eco81I, AxyI, Bse21I, Bsu36I, Eco88I, Ama87I, AvaI, BmeT110I, BsiHKCI, BsoBlm, Eco91I, BstEII, BstPI, EcoO65I, PspEI, Eco130I, BssT1I, EcoT14I, ErhI, StyI, HinlI, AcyI, BsaHI, BssNI, BstACI, Hsp92I, AspLEI, BstHHI, CfoI, HinPI1I, HspAI, HindIII, HpaII, BsiSI, HapII, Kpn2I, AccIII, Aor13HI, BseAI, Bsp13I, BspEI, MroI, MspI, BsiSI, HapII, MvaI, AjnI, BseBI, Bst2UI, BstNI, BstOI, Psp6I, PspGI, MluI, NcoI, Bsp19I, NdeI, FauNDI, NotI, CciNI, SalI, TaqI, XbaI, XhoI, BelI, CpoI, CspI, Rsr2I, RsrII, MunI, MfeI, Psp5II, PpuMI, PspPPI, Bsp1431, BfuCI, BssMI, BstKTI, BstMBI, DpnII, Kzo9I, NdeII, Sau3Aim, HinfI, MboI, BfuCI, BssMI, BstKTI, BstMBI, DpnII, Kzo9I, NdeII, Sau3Alm, BspTI, AfIII, BfrI, Bst98I, BstAFI, MspCI, Vha464I, Pfl23II, BsiWI, PspLI, Acc65I, Asp718I, VspI, AseI, PshBI, Bsp1407I, BsrGI, BstAUI, Psp1406I, AclI, NheI, AsuNHI, BmtI, Tru1I, MseI, Tru9I, BshNI, AccB1I, BanI, BspT107I, BseDI, BsaJI, BssECI, BfmI, BstSFI, SfcI, Bpu10I, BcuI, AhII, SpeI, PagI, CciI, BspHI, ReaI, TatI, XagI, BstENI, EcoNI, PsyI, AspI, PflFI, Tth111I, TasI, Sse9I, Tsp509I, TspEI, XapI, AcsI, ApoI, Bme1390I, BmrFI, BssKI, BstSCI, MspR9I, ScrFI, StyD4I, BseXI, BbvI, BstV1I, BshTI, AgeI, AsiGI, CspAI, PinAI, XmiI, AccI, FbII, NmuCI, Tsp45I, PsuI, BstX2I, BstYI, MflI, XhoII, XmaJI, AspA2I, AvrII, BinI, SatI, Fnu4HI, Fsp4HI, ItaI, BveI, Acc36I, BfuAI, BspMI, PfoI, FspBI, BfaI, MaeI, XspI, PfeI, TfiI, SsiI, AciI, BspACI, FaqI, BsiFI, BsmFI, HpyF3I, BstDEI, DdeI, SgsI, AscI, PalAI, CseI, HgaI, Lsp1109I, BbvI, BstV1I, CsiI, MabI, SexAI, PteI, BsePlm, BssHII, FokI, BstF5I, BtsCI, KflI, Sse1825I, SanDI, SaqAI, MseI, Tru1I, or Tru9I, or an isoschizomers of any of the aforesaid restriction enzymes. In some embodiments, the restriction enzyme is selected from AcuI, AiwI, AaR1, BaeI, BbsI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, EarI, EciI, Esp3I, FauI, FokI, HgaI, HphI, HpyAV, MbolI, MlyI, MnlI, Nme-AIII, PaqCI, PleI, SapI, SfaNI, AbsI, Acc65I, AgeI, AgsI, ArsI, AscI, AsiGI, AsiSI, Asp718I, AspA2I, AsuNHI, AvrII, Alw44I, AxyI, BamHI, BelI, BcnI, BglII, Bpu1102I, BlnI, BmtI, BsaI, Bse21I, BshTI, BsmBI, Bso31I, BspOI, BspTNI, BstENI, Bsp119I, Bsp120I, Bsu15I, Bsu36I, CciNI, Cfr13I, Csp6I, Cfr9I, CspAI, Ec1136II, Eco31I, Eco53kI, Eco81I, Eco91I, Eco52I, EcoICRI, EcoNI, EcoRI, Esp3I, FseI, Hin6I, HindIII, HpaII, I-CeuI, I-PpoI, I-SceI, KpnI, Kpn2I, LmnI, LpnPI, MauBI, MspI, MluI, NcoI, NdeI, NheI, NotI, PalAI, PI-PspI, PI-SceI, PinAI, Psp124BI, RgaI, RigI, SacI, SalI, SbfI, SdaI, SfaAI, SgfI, SgsI, SmaI, SrfI, Sse83871, SstI, TaqI, TspMI, XagI, XbaI, XhoI, XmaI, XmaCI, XmaJI, MunI, HinfI, BspTI, Pfl23II, VspI, Bsp1407I, Psp1406I, Tru1I, Bpu10I, BcuI, PagI, BshTI, XmaJI, PfoI, SgsI, PteI, KflI, or an isoschizomer of any of the aforesaid restriction enzymes. In some embodiments, the restriction enzyme is selected from AbsI, Acc65I, AgeI, AgsI, ArsI, AscI, AsiGI, AsiSI, Asp718I, AspA2I, AsuNHI, AvrII, Alw44I, AxyI, BamHI, BelI, BcnI, BglII, Bpu1102I, BlnI, BmtI, BsaI, Bse21I, BshTI, BsmBI, Bso31I, BspOI, BspTNI, BstENI, Bsp119I, Bsp120I, Bsu15I, Bsu36I, CciNI, Cfr13I, Csp6I, Cfr9I, CspAI, Ec1136II, Eco31I, Eco53kI, Eco81I, Eco91I, Eco52I, EcoICRI, EcoNI, EcoRI, Esp3I, FseI, Hin6I, HindIII, HpaII, I-CeuI, I-PpoI, I-SceI, KpnI, Kpn2I, LmnI, LpnPI, MauBI, MspI, MluI, NcoI, NdeI, NheI, NotI, PalAI, PI-PspI, PI-SceI, PinAI, Psp124BI, RgaI, RigI, SacI, SalI, SbfI, SdaI, SfaAI, SgfI, SgsI, SmaI, SrfI, Sse83871, SstI, TaqI, TspMI, XagI, XbaI, XhoI, XmaI, XmaCI, XmaJI, MunI, HinfI, BspTI, Pfl23II, VspI, Bsp1407I, Psp1406I, Tru1I, Bpu101, BcuI, PagI, BshTI, XmaJI, PfoI, SgsI, PteI, KflI, or an isoschizomer of any of the aforesaid restriction enzymes. In some embodiments, the restriction enzyme is AgeI, AvrII, BamHI, NheI, AscI, NotI, or an isoschizomer of any of the aforesaid restriction enzymes. In some embodiments, the restriction enzyme is AgeI, AvrII, BamHI, NheI, or an isoschizomer of any of the aforesaid restriction enzymes. In some embodiments, the restriction enzyme is AscI, NotI, or an isoschizomer of any of the aforesaid restriction enzymes. In some embodiments, the subgenomic fragments are generated from the first carrier vector or the separate carrier vectors by digesting the carrier vector or separate carrier vectors with the same type IIS restriction enzyme, e.g., a type IIS restriction enzyme chosen from, but not limited to, AcuI, AlwI, AaR1, BaeI, BbsI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, EarI, EciI, Esp3I, FauI, FokI, HgaI, HphI, HpyAV, MbolI, MlyI, MnlI, Nme-AIII, PaqCI, PleI, SapI, or SfaNI, or an isoschizomer of any of the aforesaid restriction enzymes. In some embodiments, the type IIS restriction enzyme is BsaI. In some embodiments, the type IIS restriction enzyme is BsmBI. Additional non-limiting restriction enzymes (e.g., type IIS restriction enzymes) suitable for use include, for example, AasI, Acc36I, AccB7I, AceIII, AcpII, Adel, AfiI, AjuI, AlfI, AloI, Alw26I, AlwNI, AlwXI, AmaCSI, ApaBI, ApyPI, AquII, AquIII, AquIV, ArsI, Asp10HII, BasI, Bbr7I, BbvI, BbvII, Bbv16II, Bce4I, Bce83I, BceCI, BcgI, Bco5I, Bco116I, BcoKI, BflI, BfuAI, BglI, Bli736I, Bme585I, BpiI, BplI, BpmI, BpuAI, BpuJI, BpuSI, BsAJI, Bsc4I, Bsc91I, Bsc107I, BscAI, Bse3DI, BseGI, BseKI, BseLI, BseMI, BseMII, BseXI, BseZI, BsgI, BsiYI, BslI, BslFI, Bso311, BsoMAI, Bsp24I, Bsp423I, BspBS31I, BspCNI, BspD6I, BsplS4I, BspKT5I, BspLU11III, BspMI, BspST5I, BspTNI, BspTS514I, BspWI, Bst6I, Bst12I, Bst19I, Bst71I, BstAPI, BstBS32I, BstFZ438I, BstGZ53I, BstHZ55I, BstlZ316I, BstMAI, BstMWI, BstOZ616I, BstPZ418I, BstTS5I, BstV1I, BstV2I, BstXI, CaiI, CjeI, CjePI, CspCI, CstMI, DraIII, DrdI, DseDI, Eam1104I, Eco31I, Eco5TI, EcoA4I, Eco57MI, EcoO44I, EcoP15I, Esp3I, Esp1396I, GsuI, HaeIV, Hin4I, Hpy178III, Hpy188III, HpyF10VI, LweI, MmeI, MwoI, PfiBI, PfiMI, PhaI, PpiI, PsrI, RleAI, SdiI, SfiI, SmuI, Sth1321, StsI, TaqII, TspDTI, Tsp8EI, TspGWI, Tth11111, Van91I, or VpaK11AI, or an isoschizomer of any of the aforesaid restriction enzymes. In some embodiments, the restriction enzyme recognizes a discontinuous site and is selected from, but not limited to, AasI, AccB7I, AcpII, Adel, AfiI, AjuI, AlwNI, ApaBI, ArsI, Asp10HII, BaeI, BasI, Bce4I, BceCI, BcgI, BfiI, BglI, Bsc4I, Bsc107I, BseLI, BsiYI, BslI, BspWI, BstAPI, BstH255I, BstlZ316I, BstMWI, BstXI, CaiI, BraIII, DrdI, DseDI, HpyF10VI, PflBI, PflMI, SdiI, SfiI, Tsp8EI, or Van91I, or an isoschizomer of any of the aforesaid restriction enzymes. In some embodiments, the restriction enzyme is capable of producing a 5′ overhang upon cleavage by the restriction enzyme. In some embodiments, cleavage by the restriction enzyme results in at least one or at least two (e.g., one or two) base pairs outside the 5′ overhang in its recognition site. In some embodiments, the restriction enzyme is capable of producing a 3′ overhang upon cleavage by the restriction enzyme. In some embodiments, the restriction enzyme is heat inactivatable. In some embodiments, the restriction enzyme recognizes a stretch of 4-8 base pairs, e.g., 4 base pairs, 5 base pairs, 6 base pairs, 7 base pairs, or 8 base pairs. In some embodiments, the restriction enzyme is not EcoRI.

In some embodiments, the subgenomic fragments, e.g., 2-20, 2-16, 2-12, 2-8, 2-4, 5-20, 5-16, 5-12, 5-8, 10-20, 10-16, 10-12, 15-20, 15-18, 15-16, 1, 2, 3, 4, 5, 6, 7, 8, 9, 10, 11, 12, 13, 14, 15, 16, 17, 18, 19, or 20 subgenomic fragments (e.g., 5-20, 10-20, 12-18, 14-18, or 16 subgenomic fragments), are capable of ordered assembly based on the complementarity of the 5′ overhang in one subgenomic fragment with the 3′ overhang in another subgenomic fragment to generate a baculovirus genome or baculovirus expression construct.

In some embodiments, the subgenomic fragments, e.g., 2-20, 2-16, 2-12, 2-8, 2-4, 5-20, 5-16, 5-12, 5-8, 10-20, 10-16, 10-12, 15-20, 15-18, 15-16, 1, 2, 3, 4, 5, 6, 7, 8, 9, 10, 11, 12, 13, 14, 15, 16, 17, 18, 19, or 20 subgenomic fragments (e.g., 5-20, 10-20, 12-18, 14-18, or 16 subgenomic fragments), are capable of ordered assembly based on the overlap in nucleotide sequence at the 5′ end of one subgenomic fragment with the nucleotide sequence at the 3′ end of another subgenomic fragment to generate a variant baculovirus genome or the baculovirus expression construct. In some embodiments, the overlap in nucleotide sequence between two subgenomic fragments is 4-50 (e.g., 4-45, 4-40, 4-35, 4-30, 4-25, 4-20, 4-15, 4-10, 10-50, 10-45, 10-40, 10-35, 10-30, 10-25, 10-20, 10-15, 15-50, 15-45, 15-40, 15-35, 15-30, 15-25, 15-20, 20-50, 20-45, 20-40, 20-35, 20-30, 20-25, 25-50, 25-45, 25-40, 25-35, 25-30, 30-50, 30-45, 30-40, 30-35, 35-50, 35-45, 35-40, 40-50, 40-45) base pairs, e.g., contiguous base pairs. In some embodiments, following cleavage by a selected type II restriction enzyme disclosed herein, a 5′ exonuclease creates a single-stranded region of complementarity corresponding to the overlapping nucleotide sequence between subgenomic fragments, producing subgenomic fragments capable of annealing with each other. In some embodiments, gaps between the annealed subgenomic fragments are filled in by a DNA polymerase.

In some embodiments, at least two subgenomic fragments are covalently linked, e.g., using a ligase (e.g., a T4 ligase), for example using Golden Gate Assembly, such that at least two subgenomic regions are formed. In some embodiments, at least two subgenomic fragments are covalently linked, e.g., using a ligase (e.g., a T4 ligase), for example using Golden Gate Assembly, to generate a variant baculovirus genome. In some embodiments, at least two subgenomic fragments are covalently linked, e.g., using a ligase (e.g., a T4 ligase), for example using Golden Gate Assembly, to generate a baculovirus expression construct. In some embodiments, the subgenomic fragments are ligated in a single step to generate a variant baculovirus genome or a baculovirus expression construct. The Golden Gate Assembly method is well known in the art (see, e.g., Engler et al., PLoS ONE 2008; 3:e3647; Engler et al., PLoS ONE 2009; 4:e5553; Weber et al., PLoS ONE 2011; 6:e16765). In some embodiments, Gibson Assembly™ is used to covalently link at least two subgenomic fragments to form at least two subgenomic regions, to generate a variant baculovirus genome, or to generate a baculovirus expression construct.

In some embodiments, 2-20, 2-16, 2-12, 2-8, 2-4, 5-20, 5-16, 5-12, 5-8, 10-20, 10-16, 10-12, 15-20, 15-18, 15-16, 1, 2, 3, 4, 5, 6, 7, 8, 9, 10, 11, 12, 13, 14, 15, 16, 17, 18, 19, or 20 subgenomic fragments are covalently linked, e.g., using a ligase (e.g., a T4 ligase), for example using Golden Gate Assembly or Gibson Assembly™, such that at least two subgenomic regions are formed. In some embodiments, 2-20, 2-16, 2-12, 2-8, 2-4, 5-20, 5-16, 5-12, 5-8, 10-20, 10-16, 10-12, 15-20, 15-18, 15-16, 1, 2, 3, 4, 5, 6, 7, 8, 9, 10, 11, 12, 13, 14, 15, 16, 17, 18, 19, or 20 subgenomic fragments are covalently linked, e.g., using a ligase (e.g., a T4 ligase), for example using Golden Gate Assembly or Gibson Assembly™, to generate a variant baculovirus genome. In some embodiments, 2-20, 2-16, 2-12, 2-8, 2-4, 5-20, 5-16, 5-12, 5-8, 10-20, 10-16, 10-12, 15-20, 15-18, 15-16, 1, 2, 3, 4, 5, 6, 7, 8, 9, 10, 11, 12, 13, 14, 15, 16, 17, 18, 19, or 20 subgenomic fragments are covalently linked, e.g., using a ligase (e.g., a T4 ligase), for example using Golden Gate Assembly or Gibson Assembly™, to generate the baculovirus expression construct. In some embodiments, 14-18, 14, 15, 16, 17, or 18 subgenomic fragments are covalently linked, e.g., using a ligase (e.g., a T4 ligase), for example using Golden Gate Assembly or Gibson Assembly™, such that at least two subgenomic regions are formed. In some embodiments, 14-18, 14, 15, 16, 17, or 18 subgenomic fragments are covalently linked, e.g., using a ligase (e.g., a T4 ligase), for example using Golden Gate Assembly or Gibson Assembly™, to generate a variant baculovirus genome. In some embodiments, 14-18, 14, 15, 16, 17, or 18 subgenomic fragments are covalently linked, e.g., using a ligase (e.g., a T4 ligase), for example using Golden Gate Assembly or Gibson Assembly™, to generate a baculovirus expression construct. In some embodiments, the subgenomic fragments (e.g., 16 subgenomic fragments) are ligated in a single step to generate a variant baculovirus genome or baculovirus expression construct.

In some embodiments, the covalently-linked subgenomic fragments or variant baculovirus genome are linear (e.g., linear DNA). In some embodiments, the covalently-linked subfragments or variant baculovirus genome are circular (e.g., circular DNA), for example, when present in a first carrier vector or a destination vector (e.g., BAC).

In some embodiments, the covalently-linked subgenomic fragments are present in (or cloned into) a backbone destination vector (e.g., bacmid). In some embodiments, the variant baculovirus genome is present in a backbone destination vector (e.g., bacmid). In some embodiments, the backbone destination vector comprises a stuffer nucleotide sequence flanked at the 5′ end and 3′ end by recognition sites for the restriction enzyme (e.g., type IIS restriction enzyme) used to generate the unique 5′ overhang and unique 3′ overhang of the subgenomic fragments. In some embodiments, the backbone destination vector comprises a nucleotide sequence encoding a reporter gene flanked at the 5′ end and 3′ end by recognition sites for the restriction enzyme (e.g., type IIS restriction enzyme) used to generate the unique 5′ overhang and unique 3′ overhang of the subgenomic fragments. In some embodiments, the backbone destination vector comprises a stuffer nucleotide sequence flanked at the 5′ end and 3′ end by recognition sites for a selected restriction enzyme, e.g., a selected type II restriction enzyme (e.g., a selected type IIS restriction enzyme). In some embodiments, the backbone destination vector comprises a nucleotide sequence encoding a reporter gene flanked at the 5′ end and 3′ end by recognition sites for a selected restriction enzyme, e.g., selected type II restriction enzyme (e.g., selected type IIS restriction enzyme). In some embodiments, the recognition sites differ from those used to generate the subgenomic fragments, e.g., when releasing the subgenomic fragments from a carrier vector. In some embodiments, the reporter gene is a reporter which allows for the screening (e.g., by blue-white screening) of destination vectors which comprise the covalently-linked subgenomic fragments or variant baculovirus genome. In some embodiments, the reporter gene is lacZa. In some embodiments, the backbone destination vector comprises two recognition sites for the restriction enzyme (e.g., type IIS restriction enzyme) used to generate the unique 5′ overhang and the unique 3′ overhang of the subgenomic fragments. In some embodiments, the destination vector is digested with the restriction enzyme used to generate the unique 5′ overhang and the unique 3′ overhang of the subgenomic fragments to form a region into which the covalently linked subgenomic fragments or variant baculovirus genome is inserted. In some embodiments, the backbone destination vector comprises a recognition site for a selected restriction enzyme, e.g., a selected type II restriction enzyme (e.g., a selected type IIS restriction enzyme), e.g., a recognition site which differs from that used to generate subgenomic fragments, for example, when releasing the subgenomic fragments from carrier vectors. In some embodiments, the backbone destination vector is digested with a selected restriction enzyme, e.g., a selected type II restriction enzyme (e.g., a selected type IIS restriction enzyme), which may differ from that used to generate subgenomic fragments (e.g., when releasing the subgenomic fragments from carrier vectors) to form a region into which the covalently linked subgenomic fragments or variant baculovirus genome is inserted, e.g., via Gibson Assembly™

The nucleotide sequence of a subgenomic fragment may be generated using various conventional cloning or synthetic methods. For example, all or a portion of a fragment (e.g., subfragment or subgenomic fragment) can be generated using chemical synthesis. In some embodiments, all or a portion of a subgenomic fragment is generated using a PCR-based method. In some embodiments, all or a portion of a subgenomic fragment is generated using a restriction enzyme-based method, e.g., obtaining a portion or all of a subgenomic fragment by “cutting out” a sequence of interest from a source nucleotide sequence (e.g., a vector comprising the sequence of interest). In some embodiments, a subgenomic fragment may be prepared using a combination of one or more of the aforementioned methods (or any other suitable method known in the art). In some embodiments, the subgenomic fragment is a non-templated fragment. Methods for chemically synthesizing nucleic acids and PCR are well known in the art.

In some embodiments, one or more, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 1-30, 1-20,1-10, 1-5, 1-3, or 1-2 of the subgenomic fragments comprise a nucleotide sequence which is foreign or heterologous to the variant baculovirus nucleotide sequence. In some embodiments, the heterologous nucleotide sequence comprises a sequence of interest. In some embodiments, the heterologous nucleotide sequence encodes a polypeptide of interest or nucleic acid of interest. In some embodiments, the polypeptide or nucleic acid of interest is a therapeutic polypeptide (e.g., therapeutic protein) or a therapeutic nucleotide sequence (e.g., a therapeutic RNAi). In some embodiments, the polypeptide of interest is a detectable marker gene, such as green fluorescent protein (GFP) or secreted embryonic alkaline phosphatase (SEAP). In some embodiments, the heterologous nucleotide sequence, e.g., the sequence of interest, comprises one or more adeno-associated viral (AAV) gene encoding regions and/or a payload. In some embodiments, the nucleotide sequence of the heterologous sequence is modified to remove one or more or all recognition sites of the restriction enzyme, e.g., a selected type II restriction enzyme (e.g., a selected type IIS restriction enzyme) used to generate a subgenomic fragments. In some embodiments, the heterologous nucleotide sequence is chemically synthesized. In some embodiments, the heterologous nucleotide sequence is generated by a PCR-based method. In some embodiments, the heterologous nucleotide sequence is generated by a combination of chemical synthesis and a PCR-based method. In some embodiments, the heterologous nucleotide sequence is a non-templated nucleotide sequence (e.g., a non-templated fragment). In some embodiments, the heterologous nucleotide sequence is inserted into a subgenomic fragment using available restriction enzyme sites. In some embodiments, the heterologous nucleotide sequence is inserted into a subgenomic fragment using Gibson Assembly™ (see, e.g., Gibson et al. Nat Methods 2009; 6:343-5; Gibson et al. Science 2010; 329:52-6; the contents of which are each incorporated herein by reference in their entireties).

### D. Subfragments

In some embodiments, the subgenomic fragments are further partitioned into subfragments. In some embodiments, the subgenomic fragments comprise one or more subfragments. In some embodiments, at least two (e.g., 2-20, 5-20, 10-20, 12-18, 14-18, or 16) subgenomic regions in the baculovirus expression construct comprise one or more subfragments (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 1-30, 1-20,1-10, 1-5, 1-4, 1-3, 1-2, 5-20, 10-20, 14-18, or 15-17 subfragments), e.g., 16 subfragments. In some embodiments, each subgenomic fragment comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 1-30, 1-20, 1-10, 1-5, 1-4, 1-3, 1-2, 5-20, 10-20, 14-18, or 15-17 subfragments (e.g., 16 subfragments). In some embodiments, the baculovirus expression vector comprises 2-30 subgenomic fragments comprising 2-600 subfragments, for example, 2-550, 2-500, 2-450, 2-400, 2-350, 2-300, 2-250, 2-200, 2-150, 2-100, 2-50, 2-25, 2-20, 2-18, 2-16, 2-14, 2-12, 2-10, 2-8, 2-6, 2-4, 4-600, 4-550, 4-500, 4-450, 4-400, 4-350, 4-300, 4-250, 4-200, 4-150, 4-100, 4-50, 4-25, 4-20, 4-18, 4-16, 4-14, 4-12, 4-10, 4-8, 4-6, 6-600, 6-550, 6-500, 6-450, 6-400, 6-350, 6-300, 6-250, 6-200, 6-150, 6-100, 6-50, 6-25, 6-20, 6-18, 6-16, 6-14, 6-12, 6-10, 6-8, 8-600, 8-550, 8-500, 8-450, 8-400, 8-350, 8-300, 8-250, 8-200, 8-150, 8-100, 8-50, 8-25, 8-20, 8-18, 8-16, 8-14, 8-12, 8-10, 10-600, 10-550, 10-500, 10-450, 10-400, 10-350, 10-300, 10-250, 10-200, 10-150, 10-100, 10-50, 10-25, 10-20, 10-18, 10-16, 10-14, 10-12, 12-600, 12-550, 12-500, 12-450, 12-400, 12-350, 12-300, 12-250, 12-200, 12-150, 12-100, 12-50, 12-25, 12-20, 12-18, 12-16, 12-14, 14-600, 14-550, 14-500, 14-450, 14-400, 14-350, 14-300, 14-250, 14-200, 14-150, 14-100, 14-50, 14-25, 14-20, 14-18, 14-16, 16-600, 16-550, 16-500, 16-450, 16-400, 16-350, 16-300, 16-250, 16-200, 16-150, 16-100, 16-50, 16-25, 16-20, 16-18, 18-600, 18-550, 18-500, 18-450, 18-400, 18-350, 18-300, 18-250, 18-200, 18-150, 18-100, 18-50, 18-25, 18-20, 20-600, 20-550, 20-500, 20-450, 20-400, 20-350, 20-300, 20-250, 20-200, 20-150, 20-100, 20-50, 20-25, 25-600, 25-550, 25-500, 25-450, 25-400, 25-350, 25-300, 25-250, 25-200, 25-150, 25-100, 25-50, 50-600, 50-550, 50-500, 50-450, 50-400, 50-350, 50-300, 50-250, 50-200, 50-150, 50-100, 100-600, 100-550, 100-500, 100-450, 100-400, 100-350, 100-300, 100-250, 100-200, 100-150, 150-550, 150-600, 150-500, 150-450, 150-400, 150-350, 150-300, 150-250, 150-200, 200-600, 200-550, 200-500, 200-450, 200-400, 200-350, 200-300, 200-250, 250-600, 250-550, 250-500, 250-450, 250-400, 250-350, 250-300, 300-600, 300-550, 300-500, 300-450, 300-400, 300-350, 350-600, 350-550, 350-500, 350-450, 350-400, 400-600, 400-550, 400-500, 400-450, 450-600, 450-550, 450-500, 500-600, 500-550, or 550-600 subfragments. In some embodiments, the baculovirus expression construct comprises 12-20 (e.g., 14-18, 15-17, 15, 16, or 17) subgenomic fragments comprising 12-400 (e.g., 12-350, 50-400, 50-350, 100-400, 100-350, 100-300, 150-400, 150-350, 150-300, 200-400, 200-300, 250-300, 250-275, or 250-260) subfragments. In some embodiments, the baculovirus expression construct comprises 16 subgenomic fragments comprising 256 subfragments.

In some embodiments, a subfragment is about 25-2500 bp, e.g., 25-2000, 25-1500, 25-1000, 25-750, 25-500, 25-250, 25-200, 25-150, 25-100, 25-75, 25-50, 50-2500, 50-2000, 50-1500, 50-1000, 50-750, 50-500, 50-250, 50-200, 50-150, 50-100, 50-75, 75-2500, 75-2000, 75-1500, 75-1000, 75-750, 75-500, 75-250, 75-200, 75-150, 75-100, 100-2500, 100-2000, 100-1500, 100-1000, 100-750, 100-500, 100-250, 100-200, 100-150, 150-2500, 150-2000, 150-1500, 150-1000, 150-750, 150-500, 150-250, 150-200, 200-2500, 200-2000, 200-1500, 200-1000, 200-750, 200-500, 200-250, 250-2500, 250-2000, 250-1500, 250-1000, 250-750, 250-500, 500-2500, 500-2000, 500-1500, 500-1000, 500-750, 750-2500, 750-2000, 750-1500, 750-1000, 1000-2500, 1000-2000, 1000-1500, 1500-2500, 1500-2000, or 2000-2500 bp in length. In some embodiments, a subfragment is about 25, about 50, about 75, about 100, about 150, about 200, about 250, about 300, about 350, about 400, about 450, about 500, about 550, about 600, about 650, about 700, about 750, about 800, about 850, about 900, about 950, about 1000, about 1250, about 1500, about 1750, about 2000, about 2250, or about 2500 bp in length. In some embodiments, a subfragment is about 100-1000 bp, about 250-750 bp, or about 500 bp in length.

In some embodiments, a subfragment comprises a unique 5′ overhang and a unique 3′ overhang, e.g., a unique 5′ overhang and a unique 3′ overhang described herein.

In some embodiments, a unique junction of at least two subregions within a subgenomic fragment or baculovirus expression construct comprises the unique 5′ overhang of a first subfragment and the unique 3′ overhang of a second subfragment, wherein the unique 5′ overhang of the first subfragment is complementary to the unique 3′ overhang of the second subfragment. In some embodiments, a first unique junction of at least two subregions within a subgenomic fragment or baculovirus expression construct comprises the unique 3′ overhang of a first subfragment and the unique 5′ overhang of a second subfragment, wherein the unique 3′ overhang of the first subfragment is complementary to the unique 5′ overhang of the second subfragment. In some embodiments, the unique 5′ overhang of the first subfragment is partially complementary to the unique 3′ overhang of the second subfragment. In some embodiments, the unique 5′ overhang of the first subfragment is fully complementary to the unique 3′ overhang of the second subfragment. In some embodiments, the unique 3′ overhang of the first subfragment is partially complementary to the unique 5′ overhang of the second subfragment. In some embodiments, the unique 3′ overhang of the first subfragment is fully complementary to the unique 5′ overhang of the second subfragment.

In some embodiments, the unique 5′ overhang and the unique 3′ overhang of a subfragment result from cleavage of the subfragment by a restriction enzyme. In some embodiments, the restriction enzyme, e.g., type IIS restriction enzyme, used to generate the unique 5′ overhang and unique 3′ overhang of a subfragment differs from the restriction enzyme used to generate the unique 5′ overhang and unique 3′ overhang of subgenomic fragments.

In some embodiments, the same restriction enzyme (e.g., type IIS restriction enzyme) is used to generate the unique 5′ overhang and the unique 3′ overhang of each subfragment. In some embodiments, the unique 5′ overhang and the unique 3′ overhang of each subgenomic fragment is generated by a type IIS restriction enzyme, e.g., as described supra. In some embodiments, the same restriction enzyme, e.g., the same type IIS restriction enzyme, used to generate the unique 5′ overhang and the unique 3′ overhang of each subfragment is different from the restriction enzyme used to generate the unique 5′ and 3′ overhangs of subgenomic fragments. For example, in some embodiments, BsaI is used to generate the unique overhangs in the subfragments, and BsmBI is used to generate the unique overhangs in the subgenomic fragments. In some embodiments, BsmBI is used to generate the unique overhangs in the subfragments, and BsaI is used to generate the unique overhangs in the subgenomic fragments.

In some embodiments, a subfragment comprises a nucleotide sequence at the 5′ end (e.g., a first baculovirus genome sequence) which overlaps with the nucleotide sequence at the 3′ end of another subfragment (e.g., the first baculovirus genome sequence), resulting in a region of overlapping nucleotide sequences unique to a pair of subfragments. The nucleotide sequence at the 3′ end of the subfragment (e.g., a second baculovirus genome sequence) may overlap with the nucleotide sequence at the 5′ end of another different subfragment (e.g., the second baculovirus genome sequence), resulting in another region of overlapping nucleotide sequence unique to a pair of subfragments. These regions of overlapping nucleotide sequences allow for the ordered assembly of subfragments via, e.g., a Gibson Assembly™ reaction, into a subgenomic fragment, a variant baculovirus genome, or baculovirus expression construct.

In some embodiments, the subgenomic fragments are present in a carrier vector, e.g., a “second” carrier vector (as opposed to a “first” carrier vector comprising the subgenomic fragments described herein). In some embodiments, one or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 2-30, 2-25, 2-20, 2-15, 2-10, 2-5, 5-30, 5-25, 5-20, 5-15, 5-10, 10-30, 10-25, 10-20, 10-15, 15-30, 15-25, 15-20, 20-30, 20-25, or 25-30) of the subfragments are present in a second carrier vector. In some embodiments, the second carrier vector comprises 10-20 (e.g., 12-18, 14-18, or 16) subfragments. In some embodiments, the second carrier vector comprises 16 subfragments.

In some embodiments, each of the one or more subfragments (e.g., 2-20, 12-18, 14-18, or 16 subfragments) are present in a separate second carrier vector (e.g., 2-20, 12-18, 14-18, or 16 second carrier vectors). By way of example, in one embodiment, a baculovirus expression construct comprises 16 subgenomic regions formed by 16 subgenomic fragments, wherein each subgenomic fragment is formed by 16 subfragments. In this embodiment, each of the 16 subgenomic fragments are maintained in a separate first carrier vector (i.e., total of 16 first carrier vectors), and each of the 16 subfragments which form a subgenomic fragment are maintained in a separate second carrier vector (i.e., total of 16 second carrier vectors).

Any vector (e.g., commercially available vector) suitable for cloning and maintaining the subfragments is suitable for use as a second carrier vector. In some embodiments, the backbone of the vector which is used as a second carrier vector is devoid of recognitions sites for the restriction enzyme used to generate the unique 5′ overhang and the unique 3′ overhang of the subfragments. In some embodiments, the backbone of the vector which is used as a second carrier vector is devoid of recognitions sites for the restriction enzyme used to release the subfragments for subsequent Gibson Assembly™. This, for example, avoids cutting within the vector and generating undesirable fragments when releasing the subfragment or subfragments from the second carrier vector or separate second carrier vectors with the restriction enzyme, e.g., a type II restriction enzyme (e.g., type IIS restriction enzyme). In some embodiments, if the vector comprises recognition sites for the restriction enzyme used to generate the unique 5′ and 3′ overhangs of the subgenomic regions, then the vector may be altered (e.g., domesticated) to render the recognition site(s) non-functional using conventional cloning techniques, e.g., site-directed mutagenesis.

In some embodiments, the second carrier vector comprises a nucleotide sequence which confers resistance to an antibiotic or antibiotics. In some embodiments, the second carrier vector comprises one or more (e.g., 1-3, 1-2, 1, 2, or 3) antibiotic resistance gene cassettes which confer resistance to one or more (e.g., 1-3, 1-2, 1, 2, or 3) antibiotics selected from kanamycin, ampicillin, spectinomycin, streptomycin, carbenecillin, bleomycin, erythromycin, polymyxin B, tetracycline, or chloramphenicol. In some embodiments, the second carrier vector confers resistance to an antibiotic which is not conferred by the first carrier vector comprising subgenomic fragments. In some embodiments, the first carrier vector for subgenomic fragments confers resistance to kanamycin, and the second carrier vector for subfragments confers resistance to ampicillin. In some embodiments, the first carrier vector for subgenomic fragments confers resistance to ampicillin, and the second carrier vector for subfragments confers resistance to kanamycin.

In some embodiments, the subfragments are generated from the second carrier vector or the separate second carrier vectors using the same restriction enzyme, e.g., type II restriction enzyme (e.g., type IIS restriction enzyme). In some embodiments, the restriction enzyme is a type II restriction enzyme chosen from, but not limited to, Eco31I, BsaI Bso311, BspTNI, Esp3I, BpiI, Alw26I, BsmAI, BstMAI, BmsI, BhaI, BscAI, BscUI, BsmNI, BspST5I, Bst19I, CjeP338II, LweI, Mva1312II, PhaI, SfaNI, LguI, BspQI, PsiSI, SapI, Alw44I, ApaLI, VneI, BamHI, BcnI, BglII, Bpu1102I, BlpI, Bsp1720I, CeIII, Bsp1191, AsuII, Bpu14I, BspT104I, BstBI, Csp45I, NspV, SfuI, Bsp120I, PspOMI, Bsu15I, BanIII, Bsa29I, BseCI, BshVI, BspDI, BspXI, BsuTUI, ClaI, Cfr10I, Bse118I, BsrFI, BssAI, Cfr13I, AspS9I, BmgT120I, PspPI, Sau96I, Csp6I, AfaI, CviQI, RsaNI, Eam1104I, Bst6I, Earl, EcoO1091, DraII, EcoRI, Eco47I, AvaII, Bme18I, SinI, VpaK11BI, Eco52I, BseX3I, BstZI, EagI, EcIXI, Eco81I, AxyI, Bse21I, Bsu36I, Eco88I, Ama87I, AvaI, BmeT110I, BsiHKCI, BsoBlm, Eco91I, BstEII, BstPI, EcoO65I, PspEI, Eco130I, BssT1I, EcoT14I, ErhI, StyI, HinlI, AcyI, BsaHI, BssNI, BstACI, Hsp92I, AspLEI, BstHHI, CfoI, HinPIlI, HspAI, HindIII, HpaII, BsiSI, HapII, Kpn2I, AccIII, Aor13HI, BseAI, Bsp13I, BspEI, MroI, MspI, BsiSI, HapII, MvaI, AjnI, BseBI, Bst2UI, BstNI, BstOI, Psp6I, PspGI, MluI, NcoI, Bsp19I, NdeI, FauNDI, NotI, CciNI, SalI, TaqI, XbaI, XhoI, BelI, CpoI, CspI, Rsr2I, RsrII, MunI, MfeI, Psp5II, PpuMI, PspPPI, Bsp1431, BfuCI, BssMI, BstKTI, BstMBI, DpnII, Kzo9I, NdeII, Sau3Aim, HinfI, MboI, BfuCI, BssMI, BstKTI, BstMBI, DpnII, Kzo9I, NdeII, Sau3Alm, BspTI, AfIII, BfrI, Bst98I, BstAFI, MspCI, Vha464I, Pfl23II, BsiWI, PspLI, Acc65I, Asp718I, VspI, AseI, PshBI, Bsp1407I, BsrGI, BstAUI, Psp1406I, AclI, NheI, AsuNHI, BmtI, Tru1I, MseI, Tru9I, BshNI, AccB1I, BanI, BspT107I, BseDI, BsaJI, BssECI, BfmI, BstSFI, SfcI, Bpu10I, BcuI, AhII, SpeI, PagI, CciI, BspHI, ReaI, TatI, XagI, BstENI, EcoNI, PsyI, AspI, PflFI, Tth111I, TasI, Sse9I, Tsp509I, TspEI, XapI, AcsI, ApoI, Bme1390I, BmrFI, BssKI, BstSCI, MspR9I, ScrFI, StyD4I, BseXI, BbvI, BstV1I, BshTI, AgeI, AsiGI, CspAI, PinAI, XmiI, AccI, FbII, NmuCI, Tsp45I, PsuI, BstX2I, BstYI, MflI, XhoII, XmaJI, AspA2I, AvrII, BinI, SatI, Fnu4HI, Fsp4HI, ItaI, BveI, Acc36I, BfuAI, BspMI, PfoI, FspBI, BfaI, MaeI, XspI, PfeI, TfiI, SsiI, AciI, BspACI, FaqI, BsiFI, BsmFI, HpyF3I, BstDEI, DdeI, SgsI, AscI, PalAI, CseI, HgaI, Lsp1109I, BbvI, BstV1I, CsiI, MabI, SexAI, PteI, BsePlm, BssHII, FokI, BstF5I, BtsCI, KflI, Sse1825I, SanDI, SaqAI, MseI, Tru1I, or Tru9I, or an isoschizomers of any of the aforesaid restriction enzymes. In some embodiments, the restriction enzyme is selected from AcuI, AlwI, AaR1, BaeI, BbsI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, Earl, EciI, Esp3I, FauI, FokI, HgaI, HphI, HpyAV, MbolI, MlyI, MnlI, Nme-AIII, PaqCI, PleI, SapI, SfaNI, AbsI, Acc65I, AgeI, AgsI, ArsI, AscI, AsiGI, AsiSI, Asp718I, AspA2I, AsuNHI, AvrII, Alw44I, AxyI, BamHI, BelI, BcnI, BglII, Bpu1102I, BlnI, BmtI, BsaI, Bse21I, BshTI, BsmBI, Bso31I, BspOI, BspTNI, BstENI, Bsp119I, Bsp120I, Bsu15I, Bsu36I, CciNI, Cfr13I, Csp6I, Cfr9I, CspAI, Ec1136II, Eco31I, Eco53kI, Eco81I, Eco91I, Eco52I, EcoICRI, EcoNI, EcoRI, Esp3I, FseI, Hin6I, HindIII, HpaII, I-CeuI, I-PpoI, I-SceI, KpnI, Kpn2I, LmnI, LpnPI, MauBI, MspI, MluI, NcoI, NdeI, NheI, NotI, PalAI, PI-PspI, PI-SceI, PinAI, Psp124BI, RgaI, RigI, SacI, SalI, SbfI, SdaI, SfaAI, SgfI, SgsI, SmaI, SrfI, Sse83871, SstI, TaqI, TspMI, XagI, XbaI, XhoI, XmaI, XmaCI, XmaJI, MunI, HinfI, BspTI, Pfl23II, VspI, Bsp1407I, Psp1406I, Tru1I, Bpu10I, BcuI, PagI, BshTI, XmaJI, PfoI, SgsI, PteI, KflI, or an isoschizomer of any of the aforesaid restriction enzymes. In some embodiments, the restriction enzyme is selected from AbsI, Acc65I, AgeI, AgsI, ArsI, AscI, AsiGI, AsiSI, Asp718I, AspA2I, AsuNHI, AvrII, Alw44I, AxyI, BamHI, BelI, BcnI, BglII, Bpu1102I, BlnI, BmtI, BsaI, Bse21I, BshTI, BsmBI, Bso31I, BspOI, BspTNI, BstENI, Bsp119I, Bsp120I, Bsu15I, Bsu36I, CciNI, Cfr13I, Csp6I, Cfr9I, CspAI, Ec1136II, Eco31I, Eco53kI, Eco81I, Eco91I, Eco52I, EcoICRI, EcoNI, EcoRI, Esp3I, FseI, Hin6I, HindIII, HpaII, I-CeuI, I-PpoI, I-SceI, KpnI, Kpn2I, LmnI, LpnPI, MauBI, MspI, MluI, NcoI, NdeI, NheI, NotI, PalAI, PI-PspI, PI-SceI, PinAI, Psp124BI, RgaI, RigI, SacI, SalI, SbfI, SdaI, SfaAI, SgfI, SgsI, SmaI, SrfI, Sse83871, SstI, TaqI, TspMI, XagI, XbaI, XhoI, XmaI, XmaCI, XmaJI, MunI, HinfI, BspTI, Pfl23II, VspI, Bsp1407I, Psp1406I, Tru1I, Bpu10I, BcuI, PagI, BshTI, XmaJI, PfoI, SgsI, PteI, KflI, or an isoschizomer of any of the aforesaid restriction enzymes. In some embodiments, the restriction enzyme is AgeI, AvrII, BamHI, NheI, AscI, NotI, or an isoschizomer of any of the aforesaid restriction enzymes. In some embodiments, the restriction enzyme is AgeI, AvrII, BamHI, NheI, or an isoschizomer of any of the aforesaid restriction enzymes. In some embodiments, the restriction enzyme is AscI, NotI, or an isoschizomer of any of the aforesaid restriction enzymes. In some embodiments, the subfragments are generated from the second carrier vector or the separate second carrier vectors by digesting the second carrier vector or separate second carrier vectors with the same type IIS restriction enzyme, e.g., a type IIS restriction enzyme selected from, but not limited to, AcuI, AlwI, AaR1, BaeI, BbsI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, Earl, EciI, Esp3I, FauI, FokI, HgaI, HphI, HpyAV, MbolI, MlyI, MnlI, Nme-AIII, PaqCI, PleI, SapI, or SfaNI, or an isoschizomer of any of the aforesaid restriction enzymes. In some embodiments, the type IIS restriction enzyme is BsaI. In some embodiments, the type IIS restriction enzyme is BsmBI. Additional non-limiting enzymes (e.g., type IIS restriction enzymes) suitable for use include, for example, AasI, Acc36I, AccB7I, AceIII, AcpII, Adel, AfiI, AjuI, AlfI, AloI, Alw26I, AlwNI, AlwXI, AmaCSI, ApaBI, ApyPI, AquII, AquIII, AquIV, ArsI, Asp10HII, BasI, Bbr7I, BbvI, BbvII, Bbv16II, Bce4I, Bce83I, BceCI, BcgI, Bco5I, Bco116I, BcoKI, BflI, BfuAI, BglI, Bli736I, Bme585I, BpiI, BplI, BpmI, BpuAI, BpuJI, BpuSI, BsAJI, Bsc4I, Bsc91I, Bsc107I, BscAI, Bse3DI, BseGI, BseKI, BseLI, BseMI, BseMII, BseXI, BseZI, BsgI, BsiYI, BsII, BsIFI, Bso311, BsoMAI, Bsp24I, Bsp423I, BspBS31I, BspCNI, BspD6I, BsplS4I, BspKT5I, BspLU11III, BspMI, BspST5I, BspTNI, BspTS514I, BspWI, Bst6I, Bst12I, Bst19I, Bst71I, BstAPI, BstBS32I, BstFZ438I, BstGZ53I, BstHZ55I, BstlZ316I, BstMAI, BstMWI, BstOZ616I, BstPZ418I, BstTS5I, BstV1I, BstV2I, BstXI, CaiI, CjeI, CjePI, CspCI, CstMI, DraIII, DrdI, DseDI, Eam1104I, Eco31I, Eco5TI, EcoA4I, Eco57MI, EcoO44I, EcoP15I, Esp3I, Esp1396I, GsuI, HaeIV, Hin4I, Hpy178III, Hpy188III, HpyF1OVI, LweI, MmeI, MwoI, PfiBI, PfiMI, PhaI, PpiI, PsrI, RleAI, SdiI, SfiI, SmuI, Sth132I, StsI, TaqII, TspDTI, Tsp8EI, TspGWI, Tth111II, Van91I, or VpaK11AI, or an isoschizomer of any of the aforesaid restriction enzymes. In some embodiments, the restriction enzyme recognizes a discontinuous site, for example, AasI, AccB7I, AcpII, Adel, AfiI, AjuI, AlwNI, ApaBI, ArsI, Asp10HII, BaeI, BasI, Bce4I, BceCI, BcgI, BfiI, BglI, Bsc4I, Bsc107I, BseLI, BsiYI, BsII, BspWI, BstAPI, BstH255I, BstlZ316I, BstMWI, BstXI, CaiI, BraIII, DrdI, DseDI, HpyF10VI, PflBI, PflMI, SdiI, SfiI, Tsp8EI, or Van91I, or an isoschizomer of any of the aforesaid restriction enzymes. In some embodiments, the restriction enzyme is capable of producing a 5′ overhang upon cleavage by the restriction enzyme. In some embodiments, cleavage by the restriction enzyme results in at least one or at least two (e.g., one or two) base pairs outside the 5′ overhang in its recognition site. In some embodiments, the restriction enzyme is capable of producing a 3′ overhang upon cleavage by the restriction enzyme. In some embodiments, the restriction enzyme is heat inactivatable. In some embodiments, the restriction enzyme recognizes a stretch of 4-8 base pairs, e.g., 4 base pairs, 5 base pairs, 6 base pairs, 7 base pairs, or 8 base pairs. In some embodiments, the restriction enzyme is not EcoRI.

In some embodiments, the same restriction enzyme used to generate the one or more subfragments from the second carrier vector or the separate second carrier vectors is different from the enzyme used to generate the subgenomic fragments. In some embodiments, the type IIS restriction enzyme is BsaI. In some embodiments, the type IIS restriction enzyme is BsmBI. In some embodiments, the type II restriction enzyme used to generate the subfragments is BsaI, and the type IIS restriction enzyme used to generate the subgenomic fragments is BsmBI. In some embodiments, the type II restriction enzyme used to generate the subfragments is BsmBI, and the type IIS restriction enzyme used to generate the subgenomic fragments is BsaI. Other combinations of type IIS restriction enzymes for use in generating subfragments and subgenomic fragments are also encompassed by the present disclosure.

In some embodiments, the subfragments (e.g., 2-20, 2-16, 2-12, 2-8, 2-4, 5-20, 5-16, 5-12, 5-8, 10-20, 10-16, 10-12, 15-20, 15-18, 15-16, 1, 2, 3, 4, 5, 6, 7, 8, 9, 10, 11, 12, 13, 14, 15, 16, 17, 18, 19, or 20 subgenomic fragments) are capable of ordered assembly based on the complementarity of the 5′ overhang in one subfragment with the 3′ overhang in another subfragment to generate a subgenomic fragment and/or baculovirus expression construct.

In some embodiments, the subfragments, e.g., 2-20, 2-16, 2-12, 2-8, 2-4, 5-20, 5-16, 5-12, 5-8, 10-20, 10-16, 10-12, 15-20, 15-18, 15-16, 1, 2, 3, 4, 5, 6, 7, 8, 9, 10, 11, 12, 13, 14, 15, 16, 17, 18, 19, or 20 subfragments (e.g., 5-20, 10-20, 12-18, 14-18, or 16 subfragments), are capable of ordered assembly based on the overlap in nucleotide sequence at the 5′ end of one subfragment (e.g., a baculovirus genome sequence) with the nucleotide sequence at the 3′ end of another subfragment (e.g., a baculovirus genome sequence) to generate a subgenomic fragment, variant baculovirus genome, or baculovirus expression construct. In some embodiments, the overlap in nucleotide sequence between two subfragments is 4-50 (e.g., 4-45, 4-40, 4-35, 4-30, 4-25, 4-20, 4-15, 4-10, 10-50, 10-45, 10-40, 10-35, 10-30, 10-25, 10-20, 10-15, 15-50, 15-45, 15-40, 15-35, 15-30, 15-25, 15-20, 20-50, 20-45, 20-40, 20-35, 20-30, 20-25, 25-50, 25-45, 25-40, 25-35, 25-30, 30-50, 30-45, 30-40, 30-35, 35-50, 35-45, 35-40, 40-50, 40-45) base pairs, e.g., contiguous base pairs. In some embodiments, following cleavage by a selected type II restriction enzyme disclosed herein, a 5′ exonuclease creates a single-stranded region of complementarity corresponding to the overlapping nucleotide sequence between subfragments, producing subfragments capable of annealing with each other. In some embodiments, gaps between the annealed subfragments are filled in by a DNA polymerase.

In some embodiments, at least two subfragments are covalently linked, e.g., using a ligase (e.g., a T4 ligase), for example using Golden Gate Assembly, such that one or more subgenomic regions or subgenomic fragments are formed. In some embodiments, two or more subfragments are covalently linked, e.g., using a ligase (e.g., a T4 ligase), for example using Golden Gate Assembly, to generate a variant baculovirus genome. In some embodiments, two or more subfragments are covalently linked, e.g., using a ligase (e.g., a T4 ligase), for example using Golden Gate Assembly, to generate a baculovirus expression construct. In some embodiments, the subfragments are ligated in a single step to generate one or more subgenomic fragments, a variant baculovirus genome, or a baculovirus expression construct. In some embodiments, Gibson Assembly™ is used to covalently link at least two subfragments to form at least two subgenomic regions, to generate a variant baculovirus genome, or to generate a baculovirus expression construct.

In some embodiments, 2-20, 2-16, 2-12, 2-8, 2-4, 5-20, 5-16, 5-12, 5-8, 10-20, 10-16, 10-12, 15-20, 15-18, 15-16, 1, 2, 3, 4, 5, 6, 7, 8, 9, 10, 11, 12, 13, 14, 15, 16, 17, 18, 19, or 20 subfragments are covalently linked, e.g., using a ligase (e.g., a T4 ligase), for example using Golden Gate Assembly or Gibson Assembly™, such that one or more subgenomic regions or subgenomic fragments are formed. In some embodiments, 2-20, 2-16, 2-12, 2-8, 2-4, 5-20, 5-16, 5-12, 5-8, 10-20, 10-16, 10-12, 15-20, 15-18, 15-16, 1, 2, 3, 4, 5, 6, 7, 8, 9, 10, 11, 12, 13, 14, 15, 16, 17, 18, 19, or 20 subfragments are covalently linked, e.g., using a ligase (e.g., a T4 ligase), for example using Golden Gate Assembly or Gibson Assembly™, to generate a variant baculovirus genome. In some embodiments, 2-20, 2-16, 2-12, 2-8, 2-4, 5-20, 5-16, 5-12, 5-8, 10-20, 10-16, 10-12, 15-20, 15-18, 15-16, 1, 2, 3, 4, 5, 6, 7, 8, 9, 10, 11, 12, 13, 14, 15, 16, 17, 18, 19, or 20 subfragments are covalently linked, e.g., using a ligase (e.g., a T4 ligase), for example using Golden Gate Assembly or Gibson Assembly™, to generate the baculovirus expression construct. In some embodiments, 14-18, 14, 15, 16, 17, or 18 subfragments are covalently linked, e.g., using a ligase (e.g., a T4 ligase), for example using Golden Gate Assembly or Gibson Assembly™, such that one or more subgenomic regions or subgenomic fragments are formed. In some embodiments, 14-18, 14, 15, 16, 17, or 18 subfragments are covalently linked, e.g., using a ligase (e.g., a T4 ligase), for example using Golden Gate Assembly or Gibson Assembly™, to generate a variant baculovirus genome. In some embodiments, 14-18, 14, 15, 16, 17, or 18 subfragments are covalently linked, e.g., using a ligase (e.g., a T4 ligase), for example using Golden Gate Assembly or Gibson Assembly™, to generate a baculovirus expression construct. In some embodiments, the subfragments (e.g., 16 subfragments) are ligated in a single step to generate one or more subgenomic fragments, a variant baculovirus genome, or baculovirus expression construct.

In some embodiments, the covalently-linked subfragments are linear (e.g., linear DNA). In some embodiments, the covalently-linked subfragments are circular (e.g., circular DNA), for example, when present in a second carrier vector, a first carrier vector, or a destination vector (e.g., BAC).

The nucleotide sequence of a subfragment may be generated using various conventional cloning or synthetic methods. For example, all or a portion of a fragment (e.g., subfragment or subgenomic fragment) can be generated using chemical synthesis. In some embodiments, all or a portion of a subfragment is generated using a PCR-based method. In some embodiments, the subfragment is a non-templated fragment. In some embodiments, all or a portion of a subfragment is generated using a restriction enzyme-based method, e.g., obtaining a portion or all of a subfragment by “cutting out” a sequence of interest from a source nucleotide sequence (e.g., a vector comprising the sequence of interest). In some embodiments, a subfragment may be prepared using a combination of one or more of the aforementioned methods (or any other suitable method known in the art).

In some embodiments, one or more, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 1-30, 1-20, 1-10, 1-5, 1-3, or 1-2 of the subfragments comprise a nucleotide sequence which is foreign or heterologous to the variant baculovirus nucleotide sequence. In some embodiments, the heterologous nucleotide sequence comprises a sequence of interest. In some embodiments, the heterologous nucleotide sequence encodes a polypeptide of interest or nucleic acid of interest. In some embodiments, the polypeptide or nucleic acid of interest is a therapeutic polypeptide (e.g., therapeutic protein) or a therapeutic nucleotide sequence (e.g., a therapeutic RNAi). In some embodiments, the polypeptide of interest is a detectable marker gene, such as green fluorescent protein (GFP) or secreted embryonic alkaline phosphatase (SEAP). In some embodiments, the heterologous nucleotide sequence, e.g., the sequence of interest, comprises one or more adeno-associated viral (AAV) gene encoding regions and/or a payload. In some embodiments, the nucleotide sequence of the heterologous sequence is modified to remove one or more or all recognition sites of the restriction enzyme (e.g., a selected type IIS restriction enzyme) used to generate subfragments. In some embodiments, the heterologous nucleotide sequence is chemically synthesized. In some embodiments, the heterologous nucleotide sequence is generated by a PCR-based method. In some embodiments, the heterologous nucleotide sequence is generated by a combination of chemical synthesis and a PCR-based method. In some embodiments, the heterologous nucleotide sequence is a non-templated nucleotide sequence (e.g., a non-templated fragment). In some embodiments, the heterologous nucleotide sequence is inserted into a subfragment using available restriction enzyme sites. In some embodiments, the heterologous nucleotide sequence is inserted into a subfragment using Gibson Assembly™

In some embodiments, a subfragment can be further subdivided into one or more additional fragments. In some embodiments, the one or more additional fragments are maintained on a third carrier vector. In some embodiments, the one or more additional fragments are generated from the third carrier vector or the separate third carrier vectors using the same restriction enzyme, e.g., the same type II restriction enzyme (e.g., same type IIS restriction enzyme), wherein the same restriction enzyme is different from the restriction enzyme used to generate the subgenomic fragments and the restriction enzyme used to generate the subfragments. In some embodiments, the one or more additional fragments are capable of ordered assembly based on the complementarity (partial or full complementarity) of the 5′ overhang in one of the additional fragments with the 3′ overhang in another additional fragment to generate a subfragment. In some embodiments, the one or more additional fragments are capable of ordered assembly based on the overlap in nucleotide sequence at the 5′ end of one of the additional fragments with the nucleotide sequence at the 3′ end of another of the additional fragments to generate a subfragment, e.g., via Gibson Assembly™.

### E. Variant Nucleotide Sequences

In some embodiments, the baculovirus expression constructs described herein comprise a variant nucleotide sequence relative to a nucleotide sequence in a reference baculovirus genome. In some embodiments, the baculovirus expression construct comprises a nucleotide sequence or a portion thereof of a reference baculovirus genome. In some embodiments, the baculovirus expression construct comprises a wild-type baculovirus genome (e.g., a reference baculovirus genome). In some embodiments, the baculovirus expression construct comprises a baculovirus genome which is modified relative to a reference baculovirus genome.

Modifications relative to a nucleotide sequence in a reference viral genome (e.g., reference baculovirus genome) can be introduced to generate a variant viral nucleotide sequence (e.g., variant baculovirus nucleotide sequence). Exemplary modifications include, but are not limited to, the substitution, addition, or deletion of one or more nucleotides, the introduction of one or more transgenes, the introduction of one or more enzyme recognition sites, the deletion of one or more enzyme recognition sites, and the deletion of one or more nucleotide regions (e.g., non-essential regions, such as a non-essential gene encoding region or the regulatory region of a non-essential gene (e.g., auxiliary and/or per os infectivity factor gene) encoding region). In some embodiments, a non-essential gene region (e.g., gene encoding region or regulatory region) is modified, e.g., by deletion, insertion, or mutation, to reduce or eliminate a gene product. Exemplary modifications include frameshift mutations, promoter modification, or insertion of a heterologous DNA adjacent to a non-essential gene.

Wild-type baculovirus genomes which can serve as reference baculovirus genomes are well known in the art (see, e.g., www.talk.ictvonline.org/ictv-reports/ictv_online_report/dsdna-viruses/w/baculoviridae, the contents of which are herein incorporated by reference in their entirety).

In some embodiments, the reference baculovirus genome is an alpha-baculovirus genome. In some embodiments, the reference baculovirus genome is beta-baculovirus genome. In some embodiments, the reference baculovirus genome is an delta-baculovirus genome. In some embodiments, the reference baculovirus genome is a gamma-baculovirus genome. In some embodiments, the reference baculovirus genome is the genome of an alpha-baculovirus selected from: Adoxophyes honmai nucleopolyhedrovirus (NPV), Agrotis ipsilon multiple nucleopolyhedrovirus (MNPV), Agrotis segetum NPVA, Agrotis segetum NPVB, Antheraea pernyi NPV, Anticarsia gemmatalis MNPV, Autographa californica MNPV (AcMNPV), Bombyx mori NPV (BmNPV), Buzura suppressaria NPV, Catopsilia pomona NPV, Choristoneura fumiferana DEF MNPV, Choristoneura fumiferana MNPV, Choristoneura murinana NPV, Choristoneura rosaceana NPV, Chrysodeixis chalcites NPV, Chrysodeixis includens NPV, Clanis bilineata NPV, Condylorrhiza vestigialis NPV, Cryptophlebia peltastica NPV, Cyclophragma undans NPV, Ectropis obliqua NPV, Epiphyas postvittana NPV, Euproctis pseudoconspersa NPV, Helicoverpa armigera NPV, Hemileuca species NPV, Hyphantria cunea NPV, Hyposidra talaca NPV, Lambdina fiscellaria NPV, Leucania separata NPV, Lonomia obliqua NPV, Lymantria dispar MNPV, Lymantria xylina NPV, Mamestra brassicae MNPV, Mamestra configurata NPV A, Mamestra configurata NPVB, Maruca vitrata NPV, Mythimna unipuncta NPV A, Mythimna unipuncta NPVB, Operophtera brumata NPV, Orgyia leucostigma NPV, Orgyia pseudotsugata MNPV, Oxyplax ochracea NPV, Peridroma saucia NPV, Perigonia lusca NPV, Spodoptera eridania NPV, Spodoptera exempta NPV, Spodoptera exigua multiple NPV A, Spodoptera exigua multiple NPV B, Spodoptera frugiperda MNPV, Spodoptera littoralis NPV, Spodoptera litura NPV, Sucra jujuba NPV, Thysanoplusia orichalcea NPV (ThorNPV), Trichoplusia ni SNPV, Urbanus proteus NPV, Wiseana signata NPV, or a variant (e.g., variant strain) of any of the preceding alpha-baculoviruses.

In some embodiments, the reference baculovirus genome is the genome of a beta-baculovirus selected from: Adoxophyes orana granulovirus (GV), Agrotis segetum GV, Choristoneura fumiferana GV, Clostera anachoreta GV, Clostera anastomosis GV A, Clostera anastomosis GV B, Cnaphalocrocis medinalis GV, Cryptophlebia leucotreta GV, Cydia pomonella GV, Diatraea saccharalis GV, Epinotia aporema GV, Erinnyis ello GV, Harrisina brillians GV, Mocis latipes GV, Mythimna unipuncta GV A, Mythimna unipuncta GV B, Phthorimaea operculella GV, Plodia interpunctella GV, Plutella xylostella GV, Spodoptera frugiperda GV, Spodoptera litura GV, Trichoplusia ni GV, Xestia c-nigrum GV, or a variant (e.g., variant strain) of any of the preceding beta-baculoviruses.

In some embodiments, the reference baculovirus genome is the genome of the delta-baculovirus Culex nigripalpus NPV or a variant (e.g., variant strain).

In some embodiments, the reference baculovirus genome is the genome of a gamma-baculovirus selected from Neodiprion lecontei NPV, Neodiprion sertifer NPV, or a variant (e.g., variant strain) thereof.

In some embodiments, the baculovirus expression construct comprises a bMON14272, vAce25ko, or vAclef11KO baculovirus genome. In some embodiments, the baculovirus expression construct comprises a variant bMON14272 baculovirus genome.

In some embodiments, the baculovirus expression construct comprises a variant nucleotide sequence (e.g., subfragment(s), subgenomic region(s), subgenomic fragment(s), or genome) comprising fewer restriction enzyme sites (e.g., fewer functional restriction enzyme sites) relative to the nucleotide sequence in a reference baculovirus genome. In some embodiments, the variant nucleotide sequence is a variant subregion within a subgenomic region (e.g., a region within a partitioned subgenomic region). In some embodiments, the variant nucleotide sequence is a variant subfragment. In some embodiments, the variant nucleotide sequence is a variant subgenomic region. In some embodiments, the variant nucleotide sequence is a variant subgenomic fragment. In some embodiments, the variant nucleotide sequence is a variant baculovirus genome sequence.

In some embodiments, the baculovirus expression construct comprises a variant nucleotide sequence (e.g., subfragment(s), subgenomic region(s), subgenomic fragment(s), or genome) comprising at least 1, at least 2, at least 3, at least 4, at least 5, at least 6, at least 7, at least 8, at least 9, at least 10, at least 11, at least 12, at least 13, at least 14, at least 15, at least 20, at least 25, at least 30, at least 35, at least 40, at least 45, at least 50, at least 55, at least 56, at least 57, at least 58, at least 59, or at least 60 fewer restriction enzyme sites (e.g., functional naturally occurring recognition sites), e.g., type II restriction enzyme sites (e.g., type IIS restriction enzyme sites) and/or recognition sites of one or more selected type II restriction enzymes (e.g., type IIS restriction enzymes), relative to the nucleotide sequence in a reference baculovirus genome. In some embodiments, the baculovirus expression construct comprises a variant nucleotide sequence comprising 1-60, 2-60, 4-60, 6-60, 8-60, 10-60, 15-60, 20-60, 25-60, 30-60, 35-60, 40-60, 45-60, 50-60, 55-60, 1-50, 2-50, 4-50, 6-50, 8-50, 10-50, 15-50, 20-50, 25-50, 30-50, 35-50, 40-50, 45-50, 1-40, 2-40, 4-40, 6-40, 8-40, 10-40, 15-40, 20-40, 25-40, 30-40, 35-40, 1-30, 2-30, 4-30, 6-30, 8-30, 10-30, 15-30, 20-30, 25-30, 1-20, 2-20, 4-20, 6-20, 8-20, 10-20, 15-20, 1-10, 2-10, 4-10, 6-10, or 8-10 fewer restriction enzyme sites (e.g., functional naturally occurring recognition sites), e.g., type II restriction enzyme sites (e.g., type IIS restriction enzyme sites), relative to the nucleotide sequence in a reference baculovirus genome. In some embodiments, the restriction enzyme site is a recognition site for a type II restriction enzyme chosen from, but not limited to, Eco31I, BsaI Bso311, BspTNI, Esp3I, BpiI, Alw26I, BsmAI, BstMAI, BmsI, BhaI, BscAI, BscUI, BsmNI, BspST5I, Bst19I, CjeP338II, LweI, Mva1312II, PhaI, SfaNI, LguI, BspQI, PsiSI, SapI, Alw44I, ApaLI, VneI, BamHI, BcnI, BglII, Bpu1102I, BlpI, Bsp1720I, CeIII, Bsp119I, AsuII, Bpu14I, BspT104I, BstBI, Csp45I, NspV, SfuI, Bsp120I, PspOMI, Bsu15I, BanIII, Bsa29I, BseCI, BshVI, BspDI, BspXI, BsuTUI, ClaI, Cfr10I, Bse118I, BsrFI, BssAI, Cfr13I, AspS9I, BmgT120I, PspPI, Sau96I, Csp6I, AfaI, CviQI, RsaNI, Eam11041, Bst6I, Earl, EcoO109I, DraII, EcoRI, Eco47I, AvaII, Bme18I, SinI, VpaK11BI, Eco52I, BseX3I, BstZI, EagI, EcIXI, Eco81I, AxyI, Bse21I, Bsu36I, Eco88I, Ama87I, AvaI, BmeT1101, BsiHKCI, BsoBlm, Eco91I, BstEII, BstPI, EcoO65I, PspEI, Eco130I, BssTII, EcoT14I, ErhI, StyI, HinlI, AcyI, BsaHI, BssNI, BstACI, Hsp92I, AspLEI, BstHHI, CfoI, HinPIlI, HspAI, HindIII, HpaII, BsiSI, HapII, Kpn2I, AccIII, Aor13HI, BseAI, Bsp13I, BspEI, MroI, MspI, BsiSI, HapII, MvaI, AjnI, BseBI, Bst2UI, BstNI, BstOI, Psp6I, PspGI, MluI, NcoI, Bsp19I, NdeI, FauNDI, NotI, CciNI, SalI, TaqI, XbaI, XhoI, BelI, CpoI, CspI, Rsr2I, RsrII, MunI, MfeI, Psp5II, PpuMI, PspPPI, Bsp143I, BfuCI, BssMI, BstKTI, BstMBI, DpnII, Kzo9I, NdeII, Sau3Aim, HinfI, MboI, BfuCI, BssMI, BstKTI, BstMBI, DpnII, Kzo9I, NdeII, Sau3Alm, BspTI, AfIII, BfrI, Bst98I, BstAFI, MspCI, Vha464I, Pfl23II, BsiWI, PspLI, Acc65I, Asp718I, VspI, AseI, PshBI, Bsp1407I, BsrGI, BstAUI, Psp1406I, AclI, NheI, AsuNHI, BmtI, Tru1I, MseI, Tru9I, BshNI, AccB1I, BanI, BspT107I, BseDI, BsaJI, BssECI, BfmI, BstSFI, SfcI, Bpu10I, BcuI, AhII, SpeI, PagI, CciI, BspHI, ReaI, TatI, XagI, BstENI, EcoNI, PsyI, AspI, PflFI, Tth111I, TasI, Sse9I, Tsp509I, TspEI, XapI, AcsI, ApoI, Bme1390I, BmrFI, BssKI, BstSCI, MspR9I, ScrFI, StyD4I, BseXI, BbvI, BstV1I, BshTI, AgeI, AsiGI, CspAI, PinAI, XmiI, AccI, FbII, NmuCI, Tsp45I, PsuI, BstX2I, BstYI, MflI, XhoII, XmaJI, AspA2I, AvrII, BinI, SatI, Fnu4HI, Fsp4HI, ItaI, BveI, Acc36I, BfuAI, BspMI, PfoI, FspBI, BfaI, MaeI, XspI, PfeI, TfiI, SsiI, AciI, BspACI, FaqI, BsiFI, BsmFI, HpyF3I, BstDEI, DdeI, SgsI, AscI, PalAI, CseI, HgaI, Lsp11091, BbvI, BstV1I, CsiI, MabI, SexAI, PteI, BsePlm, BssHII, FokI, BstF5I, BtsCI, KflI, Sse18251, SanDI, SaqAI, MseI, Tru1I, or Tru9I, or an isoschizomers of any of the aforesaid restriction enzymes. In some embodiments, the restriction enzyme is selected from AcuI, AlwI, AaR1, BaeI, BbsI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, Earl, EciI, Esp3I, FauI, FokI, HgaI, HphI, HpyAV, MbolI, MlyI, MnlI, Nme-AIII, PaqCI, PleI, SapI, SfaNI, AbsI, Acc65I, AgeI, AgsI, ArsI, AscI, AsiGI, AsiSI, Asp718I, AspA2I, AsuNHI, AvrII, Alw44I, AxyI, BamHI, BelI, BcnI, BglII, Bpu1102I, BlnI, BmtI, BsaI, Bse21I, BshTI, BsmBI, Bso31I, BspOI, BspTNI, BstENI, Bsp119I, Bsp120I, Bsu15I, Bsu36I, CciNI, Cfr13I, Csp6I, Cfr9I, CspAI, Ec1136II, Eco31I, Eco53kI, Eco81I, Eco91I, Eco52I, EcoICRI, EcoNI, EcoRI, Esp3I, FseI, Hin6I, HindIII, HpaII, I-CeuI, I-PpoI, I-SceI, KpnI, Kpn2I, LmnI, LpnPI, MauBI, MspI, MluI, NcoI, NdeI, NheI, NotI, PalAI, PI-PspI, PI-SceI, PinAI, Psp124BI, RgaI, RigI, SacI, SalI, SbfI, SdaI, SfaAI, SgfI, SgsI, SmaI, SrfI, Sse8387I, SstI, TaqI, TspMI, XagI, XbaI, XhoI, XmaI, XmaCI, XmaJI, MunI, HinfI, BspTI, Pfl23II, VspI, Bsp1407I, Psp1406I, Tru1I, Bpu10I, BcuI, PagI, BshTI, XmaJI, PfoI, SgsI, PteI, KflI, or an isoschizomer of any of the aforesaid restriction enzymes. In some embodiments, the restriction enzyme is selected from AbsI, Acc65I, AgeI, AgsI, ArsI, AscI, AsiGI, AsiSI, Asp718I, AspA2I, AsuNHI, AvrII, Alw44I, AxyI, BamHI, BelI, BcnI, BglII, Bpu1102I, BlnI, BmtI, BsaI, Bse21I, BshTI, BsmBI, Bso31I, BspOI, BspTNI, BstENI, Bsp1191, Bsp120I, Bsu15I, Bsu36I, CciNI, Cfr13I, Csp6I, Cfr9I, CspAI, Ec1136II, Eco31I, Eco53kI, Eco811, Eco91I, Eco52I, EcoICRI, EcoNI, EcoRI, Esp3I, FseI, Hin6I, HindIII, HpaII, I-CeuI, I-PpoI, I-SceI, KpnI, Kpn2I, LmnI, LpnPI, MauBI, MspI, MluI, NcoI, NdeI, NheI, NotI, PalAI, PI-PspI, PI-SceI, PinAI, Psp124BI, RgaI, RigI, SacI, SalI, SbfI, SdaI, SfaAI, SgfI, SgsI, SmaI, SrfI, Sse8387I, SstI, TaqI, TspMI, XagI, XbaI, XhoI, XmaI, XmaCI, XmaJI, MunI, HinfI, BspTI, Pfl23II, VspI, Bsp1407I, Psp1406I, Tru1I, Bpu10I, BcuI, PagI, BshTI, XmaJI, PfoI, SgsI, PteI, KflI, or an isoschizomer of any of the aforesaid restriction enzymes. In some embodiments, the restriction enzyme is AgeI, AvrII, BamHI, NheI, AscI, NotI, or an isoschizomer of any of the aforesaid restriction enzymes. In some embodiments, the restriction enzyme is AgeI, AvrII, BamHI, NheI, or an isoschizomer of any of the aforesaid restriction enzymes. In some embodiments, the restriction enzyme is AscI, NotI, or an isoschizomer of any of the aforesaid restriction enzymes. In some embodiments, the restriction enzyme is a restriction enzyme that results in degenerative cohesive overhangs. In some embodiments, the restriction enzyme cleaves outside of its recognition site. In some embodiments, the restriction enzyme produces staggered ends (e.g., staggered ends of 2-4 bases). In some embodiments, the restriction enzyme recognizes asymmetric DNA sequences. In some embodiments, the restriction enzyme results in unique cohesive overhangs. In some embodiments, the restriction enzyme has more than one recognition site (e.g., BstXI). In some embodiments, the restriction enzyme is a type IIS restriction enzyme. In some embodiments, the restriction enzyme is selected from, but not limited to, AcuI, AlwI, AaR1, BaeI, BbsI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, Earl, EciI, Esp3I, FauI, FokI, HgaI, HphI, HpyAV, MbolI, MlyI, MnlI, Nme-AIII, PaqCI, PleI, SapI, or SfaNI, or an isoschizomer of any of the aforesaid restriction enzymes. In some embodiments, the type IIS restriction enzyme is BsaI, BsmBI, or a combination thereof. In some embodiments, the variant baculovirus nucleotide sequence is devoid of type IIS restriction sites. Additional non-limiting restriction enzymes (e.g., type IIS restriction enzymes) suitable for use include, for example, AasI, Acc36I, AccB7I, AceIII, AcpII, Adel, AfiI, AjuI, AlfI, AloI, Alw26I, AlwNI, AlwXI, AmaCSI, ApaBI, ApyPI, AquII, AquIII, AquIV, ArsI, Asp10HII, BasI, Bbr7I, BbvI, BbvII, Bbv16II, Bce4I, Bce83I, BceCI, BcgI, Bco5I, BcoI16I, BcoKI, BflI, BfuAI, BglI, Bli736I, Bme585I, BpiI, BplI, BpmI, BpuAI, BpuJI, BpuSI, BsAJI, Bsc4I, Bsc91I, Bsc107I, BscAI, Bse3DI, BseGI, BseKI, BseLI, BseMI, BseMII, BseXI, BseZI, BsgI, BsiYI, BslI, BslFI, Bso31I, BsoMAI, Bsp24I, Bsp4231, BspBS31I, BspCNI, BspD6I, BsplS4I, BspKT5I, BspLU111I, BspMI, BspST5I, BspTNI, BspTS5141, BspWI, Bst6I, Bst12I, Bst19I, Bst711, BstAPI, BstBS32I, BstFZ438I, BstGZ53I, BstHZ55I, BstlZ316I, BstMAI, BstMWI, BstOZ616I, BstPZ418I, BstTS5I, BstV1I, BstV2I, BstXI, CaiI, CjeI, CjePI, CspCI, CstMI, DraIII, DrdI, DseDI, Eam11041, Eco31I, Eco5TI, EcoA4I, Eco57MI, EcoO44I, EcoP15I, Esp3I, Esp13961, GsuI, HaeIV, Hin4I, Hpy178III, Hpy188III, HpyF10VI, LweI, MmeI, MwoI, PfiBI, PfiMI, PhaI, PpiI, PsrI, RleAI, SdiI, SfiI, SmuI, Sth1321, StsI, TaqII, TspDTI, Tsp8EI, TspGWI, Tth111II, Van91I, or VpaK11AI, or an isoschizomer of any of the aforesaid restriction enzymes. In some embodiments, the restriction enzyme recognizes a discontinuous site, for example, AasI, AccB7I, AcpII, Adel, AfiI, AjuI, AlwNI, ApaBI, ArsI, Asp10HII, BaeI, BasI, Bce4I, BceCI, BcgI, BfiI, BglI, Bsc4I, Bsc1071, BseLI, BsiYI, BslI, BspWI, BstAPI, BstH255I, BstlZ316I, BstMWI, BstXI, CaiI, BraIII, DrdI, DseDI, HpyF10VI, PflBI, PflMI, SdiI, SfiI, Tsp8EI, or Van91I, or an isoschizomer of any of the aforesaid restriction enzymes. In some embodiments, the restriction enzyme is capable of producing a 5′ overhang upon cleavage by the restriction enzyme. In some embodiments, cleavage by the restriction enzyme results in at least one or at least two (e.g., one or two) base pairs outside the 5′ overhang in its recognition site. In some embodiments, the restriction enzyme is capable of producing a 3′ overhang upon cleavage by the restriction enzyme. In some embodiments, the restriction enzyme is heat inactivatable. In some embodiments, the restriction enzyme recognizes a stretch of 4-8 base pairs, e.g., 4 base pairs, 5 base pairs, 6 base pairs, 7 base pairs, or 8 base pairs. In some embodiments, the restriction enzyme is not EcoRI.

In some embodiments, the baculovirus expression construct comprises a variant nucleotide sequence (e.g., subfragment(s), subgenomic region(s), subgenomic fragment(s), or genome) comprising at least 1, e.g., at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 55, at least 60, at least 65, at least 70, at least 75, at least 80, at least 85, or at least 90 fewer recognition sites for a selected type II restriction enzyme (e.g., a selected type IIS restriction enzyme), or one or more selected type II restriction enzymes (e.g., one or more selected type IIS restriction enzymes), relative to the nucleotide sequence in a reference baculovirus genome. In some embodiments, the baculovirus expression construct comprises a variant nucleotide sequence (e.g., subfragment(s), subgenomic region(s), subgenomic fragment(s), or genome) comprising 1-90, 2-90, 4-90, 6-90, 8-90, 10-90, 15-90, 20-90, 25-90, 30-90, 35-90, 40-90, 45-90, 50-90, 55-90, 60-90, 65-90, 70-90, 75-90, 80-90, 85-90, 1-80, 2-80, 4-80, 6-80, 8-80, 10-80, 15-80, 20-80, 25-80, 30-80, 35-80, 40-80, 45-80, 50-80, 55-80, 60-80, 65-80, 70-80, 75-80, 1-70, 2-70, 4-70, 6-70, 8-70, 10-70, 15-70, 20-70, 25-70, 30-70, 35-70, 40-70, 45-70, 50-70, 55-70, 60-70, 65-70, 70-80, 75-80, 1-60, 2-60, 4-60, 6-60, 8-60, 10-60, 15-60, 20-60, 25-60, 30-60, 35-60, 40-60, 45-60, 50-60, 55-60, 1-50, 2-50, 4-50, 6-50, 8-50, 10-50, 15-50, 20-50, 25-50, 30-50, 35-50, 40-50, 45-50, 1-40, 2-40, 4-40, 6-40, 8-40, 10-40, 15-40, 20-40, 25-40, 30-40, 35-40, 1-30, 2-30, 4-30, 6-30, 8-30, 10-30, 15-30, 20-30, 25-30, 1-20, 2-20, 4-20, 6-20, 8-20, 10-20, 15-20, 1-10, 2-10, 4-10, 6-10, or 8-10 fewer recognition sites a selected type II restriction enzyme (e.g., a selected type IIS restriction enzyme), or one or more selected type II restriction enzymes (e.g., one or more selected type IIS restriction enzymes) relative to the nucleotide sequence in a reference baculovirus genome. In some embodiments, the variant baculovirus nucleotide sequence is devoid of recognition sites for a selected type II restriction enzyme (e.g., selected type IIS restriction enzyme). In some embodiments, the variant baculovirus nucleotide sequence is devoid of recognition sites for two or more (e.g., 2-12, 2-8, 2-4, 4-12, 4-8, 6-12, 8-12, 2, 3, 4, 5, 6, 8, 7, 9, 10, or more) selected type II restriction enzymes (e.g., selected type IIS restriction enzymes). In some embodiments, the restriction enzyme is a type II restriction enzyme chosen from, but not limited to, Eco31I, BsaI Bso311, BspTNI, Esp3I, BpiI, Alw26I, BsmAI, BstMAI, BmsI, BhaI, BscAI, BscUI, BsmNI, BspST5I, Bst19I, CjeP338II, LweI, Mva1312II, PhaI, SfaNI, LguI, BspQI, PsiSI, SapI, Alw44I, ApaLI, VneI, BamHI, BcnI, BglII, Bpu1102I, BlpI, Bsp1720I, CeIII, Bsp1191, AsuII, Bpu14I, BspT104I, BstBI, Csp45I, NspV, SfuI, Bsp120I, PspOMI, Bsu15I, BanIII, Bsa29I, BseCI, BshVI, BspDI, BspXI, BsuTUI, ClaI, Cfr10I, Bse118I, BsrFI, BssAI, Cfr13I, AspS9I, BmgT120I, PspPI, Sau96I, Csp6I, AfaI, CviQI, RsaNI, Eam11041, Bst6I, Earl, EcoO109I, DraII, EcoRI, Eco47I, AvaII, Bme18I, SinI, VpaK11BI, Eco52I, BseX3I, BstZI, EagI, EclXI, Eco81I, AxyI, Bse21I, Bsu36I, Eco88I, Ama87I, AvaI, BmeT1101, BsiHKCI, BsoBlm, Eco91I, BstEII, BstPI, EcoO65I, PspEI, Eco130I, BssT1I, EcoT14I, ErhI, StyI, HinlI, AcyI, BsaHI, BssNI, BstACI, Hsp92I, AspLEI, BstHHI, CfoI, HinPIlI, HspAI, HindIII, HpaII, BsiSI, HapII, Kpn2I, AccIII, Aor13HI, BseAI, Bsp13I, BspEI, MroI, MspI, BsiSI, HapII, MvaI, AjnI, BseBI, Bst2UI, BstNI, BstOI, Psp6I, PspGI, MluI, NcoI, Bsp19I, NdeI, FauNDI, NotI, CciNI, SalI, TaqI, XbaI, XhoI, BelI, CpoI, CspI, Rsr2I, RsrII, MunI, MfeI, Psp5II, PpuMI, PspPPI, Bsp143I, BfuCI, BssMI, BstKTI, BstMBI, DpnII, Kzo9I, NdeII, Sau3Aim, HinfI, MboI, BfuCI, BssMI, BstKTI, BstMBI, DpnII, Kzo9I, NdeII, Sau3Alm, BspTI, AfIII, BfrI, Bst98I, BstAFI, MspCI, Vha464I, Pfl23II, BsiWI, PspLI, Acc65I, Asp718I, VspI, AseI, PshBI, Bsp1407I, BsrGI, BstAUI, Psp1406I, AclI, NheI, AsuNHI, BmtI, Tru1I, MseI, Tru9I, BshNI, AccB1I, BanI, BspT107I, BseDI, BsaJI, BssECI, BfmI, BstSFI, SfcI, Bpu10I, BcuI, AhII, SpeI, PagI, CciI, BspHI, ReaI, TatI, XagI, BstENI, EcoNI, PsyI, AspI, PflFI, Tth111I, TasI, Sse9I, Tsp509I, TspEI, XapI, AcsI, ApoI, Bme1390I, BmrFI, BssKI, BstSCI, MspR9I, ScrFI, StyD4I, BseXI, BbvI, BstV1I, BshTI, AgeI, AsiGI, CspAI, PinAI, XmiI, AccI, FbII, NmuCI, Tsp45I, PsuI, BstX2I, BstYI, MflI, XhoII, XmaJI, AspA2I, AvrII, BinI, SatI, Fnu4HI, Fsp4HI, ItaI, BveI, Acc36I, BfuAI, BspMI, PfoI, FspBI, BfaI, MaeI, XspI, PfeI, TfiI, SsiI, AciI, BspACI, FaqI, BsiFI, BsmFI, HpyF3I, BstDEI, DdeI, SgsI, AscI, PalAI, CseI, HgaI, Lsp11091, BbvI, BstV1I, CsiI, MabI, SexAI, PteI, BsePlm, BssHII, FokI, BstF5I, BtsCI, KflI, Sse1825I, SanDI, SaqAI, MseI, Tru1I, or Tru9I, or an isoschizomers of any of the aforesaid restriction enzymes. In some embodiments, the restriction enzyme is selected from AcuI, AiwI, AaR1, BaeI, BbsI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, Earl, EciI, Esp3I, FauI, FokI, HgaI, HphI, HpyAV, MbolI, MlyI, MnlI, Nme-AIII, PaqCI, PleI, SapI, SfaNI, AbsI, Acc65I, AgeI, AgsI, ArsI, AscI, AsiGI, AsiSI, Asp718I, AspA2I, AsuNHI, AvrII, Alw44I, AxyI, BamHI, BelI, BcnI, BglII, Bpu1102I, BlnI, BmtI, BsaI, Bse21I, BshTI, BsmBI, Bso31I, BspOI, BspTNI, BstENI, Bsp119I, Bsp120I, Bsu15I, Bsu36I, CciNI, Cfr13I, Csp6I, Cfr9I, CspAI, Ec1136II, Eco31I, Eco53kI, Eco81I, Eco91I, Eco52I, EcoICRI, EcoNI, EcoRI, Esp3I, FseI, Hin6I, HindIII, HpaII, I-CeuI, I-PpoI, I-SceI, KpnI, Kpn2I, LmnI, LpnPI, MauBI, MspI, MluI, NcoI, NdeI, NheI, NotI, PalAI, PI-PspI, PI-SceI, PinAI, Psp124BI, RgaI, RigI, SacI, SalI, SbfI, SdaI, SfaAI, SgfI, SgsI, SmaI, SrfI, Sse83871, SstI, TaqI, TspMI, XagI, XbaI, XhoI, XmaI, XmaCI, XmaJI, MunI, HinfI, BspTI, Pfl23II, VspI, Bsp1407I, Psp1406I, Tru1I, Bpu10I, BcuI, PagI, BshTI, XmaJI, PfoI, SgsI, PteI, KflI, or an isoschizomer of any of the aforesaid restriction enzymes. In some embodiments, the restriction enzyme is selected from AbsI, Acc65I, AgeI, AgsI, ArsI, AscI, AsiGI, AsiSI, Asp718I, AspA2I, AsuNHI, AvrII, Alw44I, AxyI, BamHI, BelI, BcnI, BglII, Bpu1102I, BlnI, BmtI, BsaI, Bse21I, BshTI, BsmBI, Bso31I, BspOI, BspTNI, BstENI, Bsp119I, Bsp120I, Bsu15I, Bsu36I, CciNI, Cfr13I, Csp6I, Cfr9I, CspAI, Ec1136II, Eco31I, Eco53kI, Eco81I, Eco91I, Eco52I, EcoICRI, EcoNI, EcoRI, Esp3I, FseI, Hin6I, HindIII, HpaII, I-CeuI, I-PpoI, I-SceI, KpnI, Kpn2I, LmnI, LpnPI, MauBI, MspI, MluI, NcoI, NdeI, NheI, NotI, PalAI, PI-PspI, PI-SceI, PinAI, Psp124BI, RgaI, RigI, SacI, SalI, SbfI, SdaI, SfaAI, SgfI, SgsI, SmaI, SrfI, Sse83871, SstI, TaqI, TspMI, XagI, XbaI, XhoI, XmaI, XmaCI, XmaJI, MunI, HinfI, BspTI, Pfl23II, VspI, Bsp1407I, Psp1406I, Tru1I, Bpu10I, BcuI, PagI, BshTI, XmaJI, PfoI, SgsI, PteI, KflI, or an isoschizomer of any of the aforesaid restriction enzymes. In some embodiments, the restriction enzyme is AgeI, AvrII, BamHI, NheI, AscI, NotI, or an isoschizomer of any of the aforesaid restriction enzymes. In some embodiments, the restriction enzyme is AgeI, AvrII, BamHI, NheI, or an isoschizomer of any of the aforesaid restriction enzymes. In some embodiments, the restriction enzyme is AscI, NotI, or an isoschizomer of any of the aforesaid restriction enzymes. In some embodiments, the type IIS restriction enzyme is, or one or more type IIS restriction enzymes are, selected from, but not limited to, AcuI, AlwI, AaR1, BaeI, BbsI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, Earl, EciI, Esp3I, FauI, FokI, HgaI, HphI, HpyAV, MbolI, MlyI, MnlI, Nme-AIII, PaqCI, PleI, SapI, or SfaNI, or an isoschizomer of any of the aforesaid restriction enzymes. In some embodiments, the type IIS restriction enzyme is BsaI, BsmBI, or a combination thereof. In some embodiments, the restriction enzyme (e.g., type IIS restriction enzyme) may also be selected from one or more of, but not limited to, AasI, Acc36I, AccB7I, AceIII, AcpII, Adel, AfiI, AjuI, AlfI, AloI, Alw26I, AlwNI, AlwXI, AmaCSI, ApaBI, ApyPI, AquII, AquIII, AquIV, ArsI, Asp10HII, BasI, Bbr7I, BbvI, BbvII, Bbv16II, Bce4I, Bce83I, BceCI, BcgI, Bco5I, Bco116I, BcoKI, BflI, BfuAI, BglI, Bli736I, Bme585I, BpiI, BplI, BpmI, BpuAI, BpuJI, BpuSI, BsAJI, Bsc4I, Bsc91I, Bsc107I, BscAI, Bse3DI, BseGI, BseKI, BseLI, BseMI, BseMII, BseXI, BseZI, BsgI, BsiYI, BslI, BslFI, Bso311, BsoMAI, Bsp24I, Bsp423I, BspBS31I, BspCNI, BspD6I, BsplS4I, BspKT5I, BspLU11III, BspMI, BspST5I, BspTNI, BspTS514I, BspWI, Bst6I, Bst12I, Bst19I, Bst71I, BstAPI, BstBS32I, BstFZ438I, BstGZ53I, BstHZ55I, BstlZ316I, BstMAI, BstMWI, BstOZ616I, BstPZ418I, BstTS5I, BstV1I, BstV2I, BstXI, CaiI, CjeI, CjePI, CspCI, CstMI, DraIII, DrdI, DseDI, Eam1104I, Eco31I, Eco5TI, EcoA4I, Eco57MI, EcoO44I, EcoP15I, Esp3I, Esp1396I, GsuI, HaeIV, Hin4I, Hpy178III, Hpy188III, HpyF1OVI, LweI, MmeI, MwoI, PfiBI, PfiMI, PhaI, PpiI, PsrI, RleAI, SdiI, SfiI, SmuI, Sth132I, StsI, TaqII, TspDTI, Tsp8EI, TspGWI, Tth111II, Van91I, or VpaK11AI, or an isoschizomer of any of the aforesaid restriction enzymes.

In some embodiments, the baculovirus expression construct comprises a variant nucleotide sequence (e.g., subfragment(s), subgenomic region(s), subgenomic fragment(s), or genome) comprising (a) at least 1, e.g., at least 2, 3, 4, 5, 6, 7, 8, 9, 10, 11, 12, 13, 14,15, 16, 17, 18, 19, 20, 25, 30, 35, 40, 45, 50, 55, 60, 65, 70, 75, 80, 85, or 90 fewer recognition sites (e.g., functional recognition sites), or 1-90, 1-70, 1-50, 1-30, 1-10, 10-90, 10-70, 10-50, 10-30, 20-90, 20-70, 20-50, 20-30, 30-90, 30-70, 30-50, 40-90, 40-70, 40-50, 50-90, 50-70, 60-90, 60-70, or 70-90 fewer recognition sites, for a first selected type II restriction enzyme (e.g., type IIS restriction enzyme), e.g., a type II restriction enzyme described herein, e.g., a type IIS restriction enzyme selected from AcuI, AlwI, AaR1, BaeI, BbsI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, Earl, EciI, Esp3I, FauI, FokI, HgaI, HphI, HpyAV, MbolI, MlyI, MnlI, Nme-AIII, PaqCI, PleI, SapI, or SfaNI, or an isoschizomer of any of the aforesaid restriction enzymes, relative to the nucleotide sequence in a reference baculovirus genome, and (b) at least 1, e.g., at least 2, 3, 4, 5, 6, 7, 8, 9, 10, 11, 12, 13, 14,15, 16, 17, 18, 19, 20, 25, 30, 35, 40, 45, 50, 55, 60, 65, 70, 75, 80, 85, or 90 fewer recognition sites (e.g., functional recognition sites), or 1-90, 1-70, 1-50, 1-30, 1-10, 10-90, 10-70, 10-50, 10-30, 20-90, 20-70, 20-50, 20-30, 30-90, 30-70, 30-50, 40-90, 40-70, 40-50, 50-90, 50-70, 60-90, 60-70, or 70-90 fewer recognition sites, for a second selected type II restriction enzyme (e.g., selected type IIS restriction enzyme), e.g., a type IIS restriction enzyme selected from, e.g., AcuI, AlwI, AaR1, BaeI, BbsI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, Earl, EciI, Esp3I, FauI, FokI, HgaI, HphI, HpyAV, MbolI, MlyI, MnlI, Nme-AIII, PaqCI, PleI, SapI, or SfaNI, or an isoschizomer of any of the aforesaid restriction enzymes, relative to the nucleotide sequence in a reference baculovirus genome, wherein the first and second selected type II restriction enzymes (e.g., first and second selected type IIS restriction enzymes) are different restriction enzymes. In some embodiments, the first selected type IIS restriction enzyme is BsaI, and the second selected type IIS restriction enzyme is BsmBI. In some embodiments, the variant nucleotide sequence further comprises fewer recognition sites for a third, fourth, fifth, or larger number of selected type IIS restriction enzymes relative to the nucleotide sequence in a reference baculovirus genome. In some embodiments, the variant nucleotide sequence further comprises fewer recognition sites (e.g., functional naturally occurring recognition sites) for one or more (e.g., 1-10, 1-8, 1-5, 1-4, 1-3, 1-2, 1, 2, 3, 4, 5, 6, 7, 8, 9, or 10) selected type II restriction enzymes. In some embodiments, the variant nucleotide sequence is devoid of recognition sites of both the selected first and second, or larger number of (e.g., third, fourth, fifth, or more selected type IIS restriction enzymes) type II restriction enzymes (e.g, type IIS restriction enzymes), e.g., type II restriction enzymes described herein. Additional non-limiting enzymes (e.g., type IIS restriction enzymes) include, for example, AasI, Acc36I, AccB7I, AceIII, AcpII, Adel, AfiI, AjuI, AlfI, AloI, Alw26I, AlwNI, AlwXI, AmaCSI, ApaBI, ApyPI, AquII, AquIII, AquIV, ArsI, Asp10HII, BasI, Bbr7I, BbvI, BbvII, Bbv16II, Bce4I, Bce83I, BceCI, BcgI, Bco5I, Bco116I, BcoKI, BflI, BfuAI, BglI, Bli736I, Bme585I, BpiI, BplI, BpmI, BpuAI, BpuJI, BpuSI, BsAJI, Bsc4I, Bsc91I, Bsc107I, BscAI, Bse3DI, BseGI, BseKI, BseLI, BseMI, BseMII, BseXI, BseZI, BsgI, BsiYI, BslI, BslFI, Bso311, BsoMAI, Bsp24I, Bsp4231, BspBS311, BspCNI, BspD6I, BsplS4I, BspKT5I, BspLU11III, BspMI, BspST5I, BspTNI, BspTS514I, BspWI, Bst6I, Bst12I, Bst19I, Bst71I, BstAPI, BstBS32I, BstFZ438I, BstGZ53I, BstHZ55I, BstlZ316I, BstMAI, BstMWI, BstOZ616I, BstPZ418I, BstTS5I, BstV1I, BstV2I, BstXI, CaiI, CjeI, CjePI, CspCI, CstMI, DraIII, DrdI, DseDI, Eam11041, Eco31I, Eco5TI, EcoA4I, Eco57MI, EcoO44I, EcoP15I, Esp3I, Esp13961, GsuI, HaeIV, Hin4I, Hpy178III, Hpy188III, HpyF10VI, LweI, MmeI, MwoI, PfiBI, PfiMI, PhaI, PpiI, PsrI, RleAI, SdiI, SfiI, SmuI, Sth1321, StsI, TaqII, TspDTI, Tsp8EI, TspGWI, Tth1111I, Van91I, or VpaK11AI, or an isoschizomer of any of the aforesaid restriction enzymes.

In some embodiments, the baculovirus expression construct comprises a variant nucleotide sequence (e.g., subfragment(s), subgenomic region(s), subgenomic fragment(s), or genome) comprising at least 1, e.g., at least 2, 3, 4, 5, 6, 7, 8, 9, 10, 11, 12, 13, 14,15, 16, 17, 18, 19, 20, 25, 30, 35, 40, 45, 50, 55, 60, 65, 70, 75, 80, 85, or 90 fewer recognition sites, or 1-90, 1-70, 1-50, 1-30, 1-10, 10-90, 10-70, 10-50, 10-30, 20-90, 20-70, 20-50, 20-30, 30-90, 30-70, 30-50, 40-90, 40-70, 40-50, 50-90, 50-70, 60-90, 60-70, or 70-90 fewer recognition sites, for BsaI relative to the nucleotide sequence in a reference baculovirus genome. In some embodiments, the baculovirus expression construct comprises a variant nucleotide sequence (e.g., subfragment(s), subgenomic region(s), subgenomic fragment(s), or genome) comprising at least 1, e.g., at least 2, 3, 4, 5, 6, 7, 8, 9, 10, 11, 12, 13, 14,15, 16, 17, 18, 19, 20, 25, 30, 35, 40, 45, 50, 55, 60, 65, 70, 75, 80, 85, or 90 fewer recognition sites, or 1-90, 1-70, 1-50, 1-30, 1-10, 10-90, 10-70, 10-50, 10-30, 20-90, 20-70, 20-50, 20-30, 30-90, 30-70, 30-50, 40-90, 40-70, 40-50, 50-90, 50-70, 60-90, 60-70, or 70-90 fewer recognition sites, for BsmBI relative to the nucleotide sequence in a reference baculovirus genome. In some embodiments, the baculovirus expression construct comprises a variant nucleotide sequence (e.g., subfragment(s), subgenomic region(s), subgenomic fragment(s), or genome) comprising at least 1, e.g., at least 2, 3, 4, 5, 6, 7, 8, 9, 10, 11, 12, 13, 14,15, 16, 17, 18, 19, 20, 25, 30, 35, 40, 45, 50, 55, 60, 65, 70, 75, 80, 85, or 90 fewer recognition sites, or 1-90, 1-70, 1-50, 1-30, 1-10, 10-90, 10-70, 10-50, 10-30, 20-90, 20-70, 20-50, 20-30, 30-90, 30-70, 30-50, 40-90, 40-70, 40-50, 50-90, 50-70, 60-90, 60-70, or 70-90 fewer recognition sites, for each of BsmBI and BsaI, or the combination of BsmBI and BsaI relative to the nucleotide sequence in a reference baculovirus genome.

In some embodiments, the variant baculovirus genome is devoid of recognition sites for BsmBI, BsaI, or the combination of BsmBI and BsaI relative to the nucleotide sequence in a reference baculovirus genome.

In some embodiments, the variant baculovirus genome is devoid of recognition sites for one or more type IIS restriction enzymes (e.g., BsmBI and/or BsaI), and comprises at most 1-20, e.g., 1-10, 1-8, 1-6, 1-4, 1, 2, 3, 4, 5, 6, 7, 8, 9, 10, 11, 12, 13, 14, 15, 16, 17, 18, 19, or 20 recognition sites (e.g., engineered and/or naturally occurring sites) of one or more (e.g., 1-4, 1-3, 1-2, 1, 2, 3, 4, or 5) selected type II restriction enzymes described herein (e.g., AgeI, AvrII, BamHI, NheI, AscI, NotI, or an isoschizomer of any of the aforesaid restriction enzymes). In some embodiments, the recognition site or sites of the one or more selected type II restriction enzymes are introduced into a locus of interest (e.g., by insertion of the recognition site(s) or substitution of existing sequences to create a recognition site(s)) within the variant baculovirus genome, e.g., into a non-essential gene or regulatory region of a non-essential gene described herein, optionally wherein the variant baculovirus genome is devoid of naturally occurring recognition sites for the one or more selected type II restriction enzymes.

In some embodiments, the variant nucleotide sequence comprises at least one non-naturally occurring, e.g., engineered, functional restriction enzyme site (e.g., type II restriction enzyme site (e.g., a type IIS restriction enzyme site). In some embodiments, the variant baculovirus nucleotide sequence comprises 1-20, e.g., 1-10, 1-8, 1-6, 1-4, 1, 2, 3, 4, 5, 6, 7, 8, 9, 10, 11, 12, 13, 14, 15, 16, 17, 18, 19, or 20 non-naturally occurring, e.g., engineered, restriction enzyme sites of one or more (e.g., at least about 1-4, 1-3, 1-2, 1, 2, 3, 4, or 5) selected type II restriction enzymes (e.g., selected type IIS restriction enzymes). In some embodiments, the variant baculovirus nucleotide sequence is devoid of naturally occurring sites for one or more (e.g., about 1-4, 1-3, 1-2, 1, 2, 3, 4, or 5) selected type II restriction enzymes (e.g., selected type IIS restriction enzymes), and comprises at least about 1-20, e.g., at least about 1-10, 1-8, 1-6, 1-4, 1, 2, 3, 4, 5, 6, 7, 8, 9, 10, 11, 12, 13, 14, 15, 16, 17, 18, 19, or 20 non-naturally occurring, e.g., engineered, restriction enzyme sites for one or more selected type II restriction enzymes (e.g., selected type IIS restriction enzymes). In some embodiments, the non-naturally occurring restriction enzyme site is present at a different location compared to the wild-type baculovirus genome.

In some embodiments, the baculovirus expression construct comprises a nucleotide sequence (e.g., subfragment(s), subgenomic region(s), subgenomic fragment(s), or genome) which is at least 30% identical, for example, at least 40% identical, at least 50% identical, at least 60% identical, at least 70% identical, at least 80% identical, at least 90% identical, at least 91% identical, at least 92% identical, at least 93% identical, at least 94% identical, at least 95% identical, at least 96% identical, at least 97% identical, at least 98% identical, or at least 99% identical, or 30-99%, 30-95%, 30-90%, 30-85%, 30-80%, 30-75%, 30-70%, 30-65%, 30-60%, 30-55%, 30-50%, 30-45%, 30-40%, 30-35%, 40-99%, 40-95%, 40- 90%, 40-85%, 40-80%, 40-75%, 40-70%, 40-65%, 40-60%, 40-55%, 40-50%, 40-45%, 50-99%, 50-95%, 50-90%, 50-85%, 50-80%, 50-75%, 50-70%, 50-65%, 50-60%, 50-55%, 60-99%, 60-95%, 60-90%, 60- 85%, 60-80%, 60-75%, 60-70%, 60-65%, 70-99%, 70-95%, 70-90%, 70-85%, 70-80%, 70-75%, 80-99%, 80-95%, 80-90%, 80-85%, 90-99%, 90-95%, or 95-99% identical, to the corresponding nucleotide sequence of the reference baculovirus genome.

Also provided herein are variant baculovirus genomes. In some embodiments, the size of the variant baculovirus genome (e.g., as measured in base pairs) is at least 1% larger, e.g., at least 5%, at least 10%, at least 20%, at least 30%, at least 40%, at least 50%, at least 60%, at least 70%, at least 80%, at least 90%, at least 100%, at least 150%, at least 200%, at least 250%, at least 300%, at least 350%, at least 400%, at least 450%, or at least 500% larger than that of the reference viral genome. In some embodiments, the size of the variant baculovirus genome is 1-500% larger, e.g., 1-450%, 1-400%, 1-350%, 1-300%, 1-250%, 1-200%, 1-150%, 1-100%, 1-50%, 1-25%, 1-10%, 1-5%, 5-500%, 5-450%, 5-400%, 5-350%, 5-300%, 5-250%, 5-200%, 5-150%, 5-100%, 5-50%, 5-25%, 5-10%, 10-500%, 10-450%, 10-400%, 10-350%, 10-300%, 10-250%, 10-200%, 10-150%, 10-100%, 10-50%, 10-25%, 10-20%, 20-500%, 20-450%, 20-400%, 20-350%, 20-300%, 20-250%, 20-200%, 20-150%, 20-100%, 20-50%, 20-25%, 30-500%, 30-450%, 30-400%, 30-350%, 30-300%, 30-250%, 30-200%, 30-150%, 30-100%, 30-50%, 40-500%, 40-450%, 40-400%, 40-350%, 40-300%, 40-250%, 40-200%, 40-150%, 40-100%, 40-50%, 50-500%, 50-450%, 50-400%, 50-350%, 50-300%, 50-250%, 50-200%, 50-150%, 50-100%, 100-500%, 100-450%, 100-400%, 100-350%, 100-300%, 100-250%, 100-200%, 100-150%, 200-500%, 200-450%, 200-400%, 200-350%, 200-300%, 200-250%, 300-500%, 300-450%, 300-400%, or 300-350% larger than that of the reference viral genome.

In some embodiments, the size of the variant baculovirus genome (e.g., as measured in base pairs) is at least 1% smaller, e.g., at least 5%, at least 10%, at least 20%, at least 30%, at least 40%, at least 50%, at least 60%, at least 70%, at least 80%, or at least 90% smaller than that of the reference viral genome. In some embodiments, the size of the variant baculovirus genome is 1-90%, 1-80%, 1-70%, 1-60%, 1-50%, 1-40%, 1-30%, 1-20%, 1-10%, 10-90%, 10-80%, 10-70%, 10-60%, 10-50%, 10-40%, 10-30%, 10-20%, 20-90%, 20-80%, 20-70%, 20-60%, 20-50%, 20-40%, 20-30%, 30-90%, 30-80%, 30-70%, 30-60%, 30-50%, 30-40%, 40-90%, 40-80%, 40-70%, 40-60%, 40-50%, 50-90%, 50-80%, 50-70%, 50- 60%, 60-90%, 60-80%, 60-70%, 70-90%, 70-80%, or 80-90% smaller than that of the reference viral genome.

In some embodiments, the size of the variant baculovirus genome is equal or about equal (e.g., ±5-10%) to that of the reference viral genome (e.g., an AcMNPV baculovirus genome).

In some embodiments, the variant baculovirus genome is at least 10 kilobases (kb), for example, at least 15 kb, at least 20 kb, at least 25 kb, at least 30 kb, at least 35 kb, at least 40 kb, at least 50 kb, at least 55 kb, at least 60 kb, at least 65 kb, at least 70 kb, at least 75 kb, at least 80 kb, at least 85 kb, at least 90 kb, at least 95 kb, at least 100 kb, at least 120 kb, at least 140 kb, at least 160 kb, at least 180 kb, at least 200 kb, at least 250 kb, at least 300 kb, at least 350 kb, or at least 400 kb in size. In some embodiments, the variant baculovirus genome is 1-500 kb, e.g., 1-450 kb, 1-400 kb, 1-350 kb, 1-300 kb, 1-250 kb, 1-200 kb, 1-150 kb, 1-100 kb, 1-50 kb, 50-500 kb, 50-450 kb, 50-400 kb, 50-350 kb, 50-300 kb, 50-250 kb, 50-200 kb, 50-150 kb, 50-100 kb, 100-500 kb, 100-450 kb, 100-400 kb, 100-350 kb, 100-300 kb, 100-250 kb, 100-200 kb, 100-150 kb, 150-500 kb, 150-450 kb, 150-400 kb, 150-350 kb, 150-300 kb, 150-250 kb, 150-200 kb, 200-500 kb, 200-450 kb, 200-400 kb, 200-350 kb, 200-300 kb, 200-250 kb, 250-500 kb, 250-450 kb, 250-400 kb, 250-350 kb, 250-300 kb, 300-500 kb, 300-450 kb, 300-400 kb, 300-350 kb, 350-500 kb, 350-450 kb, 350-400 kb, 400-500 kb, 400-450 kb, or 450-500 kb in size.

In some embodiments, the variant baculovirus genome has 1-1000 (e.g., 1-950, 1-900, 1-850, 1-800, 1-750, 1-700, 1-650, 1-600, 1-550, 1-500, 1-450, 1-300, 1-250, 1-200, 1-150, 1-100, 1-50, 1-25, 1-10, or 1-5) nucleotide differences (e.g., substitutions, deletions, and/or insertions of nucleotide sequences) relative to the reference viral genome sequence. In some embodiments, the nucleotide differences between the variant baculovirus genome and the reference viral genome sequence are silent differences (e.g., silent mutations) when located within the coding region of a baculovirus gene. Accordingly, in some embodiments, the difference(s) in nucleotide sequence between the variant baculovirus genome and reference genome sequence at one or more or all gene loci result in the same amino acid sequence between the encoded gene in the variant baculovirus genome and the reference viral genomes. In some embodiments, the difference(s) in nucleotide sequence between the variant baculovirus genome and reference genome sequence at one or more gene loci result in a conservative amino acid difference(s) (e.g., conservative substitutions), which are expected to have less of an impact on the expression and/or function of the encoded protein than a non-conservative amino acid difference(s).

In some embodiments, modifications to remove one or more recognition sites for a restriction enzyme (e.g., type IIS restriction enzyme) in a variant baculovirus genome are selected based on the corresponding nucleotide sequence of a baculovirus genome of the same genus as the reference baculovirus genome. By way of example, in some embodiments, if AcMNPV is the reference baculovirus genome for generating the variant baculovirus genome, then the nucleotide sequence of another alpha-baculovirus (e.g., BmNPV, ThorNPV) may be referenced to determine what substitution or substitutions may have a minimal impact when introduced at the position or positions of interest in the variant baculovirus genome. Without being bound by theory, it is expected that, if the nucleotide sequence at a position of interest is not conserved between genomes of the reference baculovirus and a different baculovirus of the same genus (e.g., a different species of baculovirus in the same genus), then a substitution to reflect the nucleotide sequence in the genome of the different baculovirus is expected to have minimal functional impact on the variant baculovirus genome.

In some embodiments, a variant sequence of a baculovirus gene of interest may replace the corresponding wild-type sequence of the gene of interest in the reference viral genome. In some embodiments, the variant sequence of a baculovirus gene of interest may be generated by introducing the desired change or changes (e.g., substitutions, insertions, and/or deletions) directly into the nucleotide sequence during chemical synthesis of the relevant portion of the genome (e.g., during chemical synthesis of a subgenomic fragment or subfragment). In some embodiments, the baculovirus expression construct may differ from a reference baculovirus genome with respect to one or more baculovirus gene encoding sequences or one or more regulatory sequences (e.g., enhancers, promoters, introns, 5′UTR, or 3′UTR).

In some embodiments, the variant nucleic acid sequence (e.g., subfragment(s), subgenomic region(s), subgenomic fragment(s), or genome) differs from that of the reference viral genome by the introduction of one or more recognition sites, e.g., for an enzyme or enzymes. In some embodiments, the variant nucleic acid sequence (e.g., subfragment(s), subgenomic region(s), subgenomic fragment(s), or genome) may differ from the reference viral genome by the introduction of one or more restriction enzyme sites. In some embodiments, the one or more restriction enzyme sites are introduced to facilitate the introduction or deletion of a nucleotide sequence of interest (e.g., introduction of a heterologous nucleotide sequence such as a transgene, deletion of a non-essential baculovirus gene, such as an auxiliary or per os infectivity factor baculovirus gene, or deletion of a non-essential region or regions (e.g., regulatory regions) in the reference viral genome).

In some embodiments, the variant nucleic acid sequence (e.g., subfragment(s), subgenomic region(s), subgenomic fragment(s), or genome) differs from the reference viral genome by the introduction of one or more foreign or heterologous nucleotide sequences. For instance, in some embodiments, the variant nucleic acid sequence may comprise nucleotides encoding one or more AAV genes and/or a payload to facilitate the production of AAV particles, as described herein. In some embodiments, the variant nucleic acid sequence may comprise a nucleotide sequence encoding a polypeptide or nucleic acid of interest (e.g., a therapeutic polypeptide or a therapeutic nucleic acid). In some embodiments, the nucleotide sequence encoding a polypeptide or nucleic acid of interest is operably linked to regulatory sequences, such as one or more enhancers and/or one or more promoters.

In some embodiments, the variant nucleic acid sequence (e.g., subfragment(s), subgenomic region(s), subgenomic fragment(s), or genome) differs from the reference viral genome by the deletion of one or more nucleotides or stretches of nucleotides.

In some embodiments, the variant nucleic acid sequence (e.g., subfragment(s), subgenomic region(s), subgenomic fragment(s), or genome) comprises a modification, such as an insertion, deletion, substitution, or mutation (e.g., frameshift mutation), in a non-essential baculovirus locus, e.g., a non-essential baculovirus gene (e.g., auxiliary or per os infectivity factor gene) and/or regulatory region of the non-essential baculovirus gene. For example, in some embodiments, the synthetic viral genome differs from the reference viral genome by a modification, e.g., insertion, deletion, or substitution, of one or more non-essential genes or regions, for example, in one or more non-essential genes or regions disclosed in International Patent Publication No. WO2014/122629, the contents of which are incorporated by reference in its entirety. In some embodiments, the modification of the non-essential region or genes does not significantly impact the production of baculovirus or ability of the baculovirus to replicate or infect a cell. In some embodiments, the modification, e.g., insertion, deletion, or substitution, results in inactivation of a non-essential baculovirus gene (e.g., auxiliary or per os infectivity factor gene) and/or regulatory region of the non-essential baculovirus gene. In some embodiments, the variant baculovirus nucleotide sequence of one or more of subgenomic fragments or subfragments comprises a disruption, e.g., a mutation (e.g., frameshift mutation), a deletion, an insertion, or inactivation, of a non-essential gene and/or regulatory region of the non-essential gene (e.g., auxiliary or per os infectivity factor gene). In some embodiments, the modification is an alteration that results in a reduction or elimination of a gene product, e.g., deletion, insertion, mutation (e.g., frameshift mutation), promoter modification, or insertion of a heterologous DNA adjacent to a non-essential gene. In some embodiments, the modification is introduced by chemical synthesis or PCR, e.g., PCR-based site-directed mutagenesis. In some embodiments, the non-essential baculovirus gene is selected from 1-64, 1-60, 1-55, 1-50, 1-45, 1-40, 1-35, 1-30, 1-25, 1-20, 1-15, 1-10, 1-5, 1-4, 1-3, 1-2, 2-64, 2-60, 2-55, 2-50, 2-45, 2-40, 2-35, 2-30, 2-25, 2-20, 2-15, 2-10, 2-5, 2-4, 2-3, 3-64, 3-60, 3-55, 3-50, 3-45, 3-40, 3-35, 3-30, 3-25, 3-20, 3-15, 3-10, 3-5, 3-4, 4-64, 4-60, 4-55, 4-50, 4-45, 4-40, 4-35, 4-30, 4-25, 4-20, 4-15, 4-10, 4-5, 5-64, 5-60, 5-55, 5-50, 5-45, 5-40, 5-35, 5-30, 5-25, 5-20, 5-15, 5-10, 10-64, 10-60, 10-55, 10-50, 10-45, 10-40, 10-35, 10-30, 10-25, 10-20, 10-15, 15-64, 15-60, 15-55, 15-50, 15-45, 15-40, 15-35, 15-30, 15-25, 15-20, 20-64, 20-60, 20-55, 20-50, 20-45, 20-40, 20-35, 20-30, 20-25, 25-64, 25-60, 25-55, 25-50, 25-45, 25-40, 25-35, 25-30, 30-64, 30-60, 30-55, 30-50, 30-45, 30-40, 30-35, 35-64, 35-60, 35-55, 35-50, 35-45, 35-40, 40-64, 40-60, 40-55, 40-50, 40-45, 45-64, 45-60, 45-55, 45-50, 50-64, 50-60, 50-55, 55-64, 55-60, 60-64, 1, 2, 3, 4, 5, 6, 7, 8, 9, 10, 12, 14, 16, 18, 20, 25, 30, 35, 40, 45, 50, 55, 60, 62, or all of Ptp, Bro, Ctx, orf603, polyhedrin, Get, bv/odv-e26, ac18, pif-2, env-prot, iap-1, Sod, Fgf, v-ubi, p43, odv-e66, gp37, odv-nc42, ac69, iap-2, pnk/pnl, ac91, odv-e28, pif-4, pif-3, pif-1, pk-2, chiA, v-cath, pp34, 94K, p26, p10, p74, ac145, odv-e56, ac150, ac11, ac30, Gta, ac63, 15k, ac97, ac121, ac140, ac146, ac149, hispP, ac44, ac47, ac84, ac112/113 Nt, ac112/113Ct, ac118, ac122, ie-01, ac152, Pena, ac56, hcf-1, ac85, cg30, ac116, and ac117. In some embodiments, the non-essential baculovirus gene is selected from 1, 2, 3, 4, 6, 7, 8, 9, 10, 11, 12, 13, 14, 15, 16, 17, 18, 19, 20, or 21 of egt, p74 (PIF0), p26, SOD, ChiA, v-cath, p10, polyhedrin, ctx, odv-e56, PIF1, PIF2, PIF3, PIF4, PIF5, Tn7, AcORF-91, AcORF-108, AcORF-52, v-ubi, or p94. In some embodiments, the non-essential gene is v-cath. In some embodiments, the baculovirus expression construct comprises a modified baculovirus genome, e.g., a modified baculovirus genome having a deletion in at least one non-essential gene, e.g., having a deletion in a polyhedrin (pohH) locus.

In some embodiments, the baculovirus expression construct or variant baculovirus genome comprises a modification, e.g., an insertion, deletion, substitution, or mutation (e.g., frame-shift mutation) in the following loci of the variant baculovirus genome: (i) v-cath and egt, (ii) v-cath, egt, and SOD1, (iii) chiA, v-cath, egt, p26, p10, p74, and SOD, (iv) chiA, v-cath, egt, p26, p10, p74, and SOD, or (v) chiA, v-cath, egt, p26, p10, p74, SOD, AcORF-91, and AcORF-108. In some embodiments, the modification comprises a deletion of a chiA gene, a v-cath gene, a p26 gene, a p10 gene, and/or a p74 gene, or a portion thereof. In some embodiments, the modification comprises an insertion of a heterologous sequence in the non-essential gene or adjacent region. In some embodiments, the modification comprises one or more mutations in the non-essential gene or adjacent region. In some embodiments, the non-essential genes are present near (e.g., downstream or upstream) of a homologous repeat region 5 (hr5).

In some embodiments, the location of a baculovirus gene of interest may be altered in the variant baculovirus genome relative to a reference viral genome. In some embodiments, the location of a baculovirus gene of interest in a variant baculovirus genome is altered such that the baculovirus gene of interest replaces the locus of a non-essential baculovirus gene (e.g., auxiliary or per os infectivity factor gene) or non-essential region (e.g., the regulatory region of a non-essential gene). The swapping of locations of a baculovirus gene of interest can be achieved, for example, by altering the order of assembly of a subgenomic fragment or a subfragment comprising the nucleotide sequence encoding the baculovirus gene of interest by designing the unique 5′ and 3′ overhangs of said subgenomic fragment or subfragment such that they are complementary (partially or fully complementary) to the unique 5′ and 3′ overhangs of subgenomic fragments or subfragments immediately proximal (e.g., adjacent) to the desired destination of the baculovirus gene of interest.

In some embodiments, at least a portion of the variant baculovirus nucleotide sequence (e.g., subfragment(s), subgenomic region(s), subgenomic fragment(s), or genome) is chemically synthesized, e.g., using art-recognized methods. In some embodiments, at least 10%, for example, at least 20%, at least 25%, at least 30%, at least 35%, at least 40%, at least 45%, at least 50%, at least 55%, at least 60%, at least 70%, at least 80%, at least 90%, at least 95%, 20-100%, 30-100%, 40-100%, 50-100%, 60-100%, 70-100%, 80-100%, 90-100%, 95-100%, 20-80%, 40-80%, 60-80%, 40-80%, 60-80%, 20-60%, 40-60%, 20-40%, or 25-50% of the variant baculovirus nucleotide sequence (e.g., subfragment(s), subgenomic region(s), subgenomic fragment(s), or genome) is chemically synthesized. In some embodiments, an entire variant baculovirus genome sequence (i.e., 100%) is chemically synthesized, e.g., assembled from subgenomic fragments or subfragments which are entirely chemically synthesized. In some embodiments, the subfragment(s), subgenomic fragment(s), or genome is non-templated.

In some embodiments, a baculovirus expression construct comprising the variant baculovirus nucleotide sequence (e.g., subfragment(s), subgenomic region(s), subgenomic fragment(s), or genome) described herein is replication competent. In some embodiments, a baculovirus expression construct comprising the variant baculovirus nucleotide sequence is capable of producing functional baculovirus. In some embodiments, a baculovirus expression construct comprising the variant baculovirus nucleotide sequence described herein is capable of producing at least 50% (e.g., at least 55%, at least 60%, at least 65%, at least 70%, at least 75%, at least 80%, at least 85%, at least 90%, at least 95%, at least 100%, at least 150%, at least 200%, 50-200%, 50-150%, 50-100%, 75-200%, 75-150%, 75-100%, 100-200%, 100-150%, or 150-200%) of the baculovirus produced by a reference viral genome. In some embodiments, a baculovirus expression construct comprising the variant baculovirus nucleotide sequence described herein is capable of producing 80-120% (e.g., 85-115%, 90-110%, 95-105%, 95%, 96%, 97%, 98%, 99%, 100%, 101%, 102%, 103%, 104%, 105%) of the baculovirus produced by the reference viral genome. In some embodiments, the amount of baculovirus produced is determined by measuring viral titer (e.g., TCID50), e.g., as determined using qPCR.

### Plurality of Fragments

Also provided herein is a plurality of fragments, e.g., a plurality of subgenomic fragments described herein or a plurality of subfragments described herein.

In some embodiments, each fragment of the plurality comprises a unique 5′ overhang and a unique 3′ overhang and a variant baculovirus nucleotide sequence. In some embodiments, one or more fragments of the plurality comprises a variant baculovirus nucleotide sequence.

In some embodiments, each fragment (e.g., a subfragment or subgenomic fragment described herein) of the plurality comprises at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 56, at least 57, at least 58, at least 59, or at least 60 fewer restriction enzyme sites, e.g., type II restriction enzyme sites (e.g., type IIS restriction enzyme sites) relative to the nucleotide sequence in a reference baculovirus genome described herein (e.g., a wild-type baculovirus genome). In some embodiments, each fragment of the plurality of fragments comprises a variant nucleotide sequence comprising 1-60, 2-60, 4-60, 6-60, 8-60, 10-60, 15-60, 20-60, 25-60, 30-60, 35-60, 40-60, 45-60, 50-60, 55-60, 1-50, 2-50, 4-50, 6-50, 8-50, 10-50, 15-50, 20-50, 25-50, 30-50, 35-50, 40-50, 45-50, 1-40, 2-40, 4-40, 6-40, 8-40, 10-40, 15-40, 20-40, 25-40, 30-40, 35-40, 1-30, 2-30, 4-30, 6-30, 8-30, 10-30, 15-30, 20-30, 25-30, 1-20, 2-20, 4-20, 6-20, 8-20, 10-20, 15-20, 1-10, 2-10, 4-10, 6-10, or 8-10 fewer restriction enzyme sites, e.g., type II restriction enzyme sites (e.g., type IIS restriction enzyme sites) relative to the nucleotide sequence in a reference baculovirus genome. In some embodiments, the restriction enzyme site is a recognition site of a type II restriction enzyme chosen from, but not limited to, Eco31I, BsaI Bso311, BspTNI, Esp3I, BpiI, Alw26I, BsmAI, BstMAI, BmsI, BhaI, BscAI, BscUI, BsmNI, BspST5I, Bst19I, CjeP338II, LweI, Mva1312II, PhaI, SfaNI, LguI, BspQI, PsiSI, SapI, Alw44I, ApaLI, VneI, BamHI, BcnI, BglII, Bpu1102I, BlpI, Bsp1720I, CeIII, Bsp1191, AsuII, Bpu14I, BspT104I, BstBI, Csp45I, NspV, SfuI, Bsp120I, PspOMI, Bsu15I, BanIII, Bsa29I, BseCI, BshVI, BspDI, BspXI, BsuTUI, ClaI, Cfr10I, Bse118I, BsrFI, BssAI, Cfr13I, AspS9I, BmgT120I, PspPI, Sau96I, Csp6I, AfaI, CviQI, RsaNI, Eam11041, Bst6I, Earl, EcoO109I, DraII, EcoRI, Eco47I, AvaII, Bme18I, SinI, VpaK11BI, Eco52I, BseX3I, BstZI, EagI, EclXI, Eco81I, AxyI, Bse21I, Bsu36I, Eco88I, Ama87I, AvaI, BmeT1101, BsiHKCI, BsoBlm, Eco91I, BstEII, BstPI, EcoO65I, PspEI, Eco130I, BssT1I, EcoT14I, ErhI, StyI, HinlI, AcyI, BsaHI, BssNI, BstACI, Hsp92I, AspLEI, BstHHI, CfoI, HinPIlI, HspAI, HindIII, HpaII, BsiSI, HapII, Kpn2I, AccIII, Aor13HI, BseAI, Bsp13I, BspEI, MroI, MspI, BsiSI, HapII, MvaI, AjnI, BseBI, Bst2UI, BstNI, BstOI, Psp6I, PspGI, MluI, NcoI, Bsp19I, NdeI, FauNDI, NotI, CciNI, SalI, TaqI, XbaI, XhoI, BelI, CpoI, CspI, Rsr2I, RsrII, MunI, MfeI, Psp5II, PpuMI, PspPPI, Bsp143I, BfuCI, BssMI, BstKTI, BstMBI, DpnII, Kzo9I, NdeII, Sau3Aim, HinfI, MboI, BfuCI, BssMI, BstKTI, BstMBI, DpnII, Kzo9I, NdeII, Sau3Alm, BspTI, AfIII, BfrI, Bst98I, BstAFI, MspCI, Vha4641, Pfl23II, BsiWI, PspLI, Acc65I, Asp718I, VspI, AseI, PshBI, Bsp1407I, BsrGI, BstAUI, Psp1406I, AclI, NheI, AsuNHI, BmtI, Tru1I, MseI, Tru9I, BshNI, AccB1I, BanI, BspT107I, BseDI, BsaJI, BssECI, BfmI, BstSFI, SfcI, Bpu10I, BcuI, AhII, SpeI, PagI, CciI, BspHI, ReaI, TatI, XagI, BstENI, EcoNI, PsyI, AspI, PflFI, Tth111I, TasI, Sse9I, Tsp509I, TspEI, XapI, AcsI, ApoI, Bme1390I, BmrFI, BssKI, BstSCI, MspR9I, ScrFI, StyD4I, BseXI, BbvI, BstV1I, BshTI, AgeI, AsiGI, CspAI, PinAI, XmiI, AccI, FbII, NmuCI, Tsp45I, PsuI, BstX2I, BstYI, MfII, XhoII, XmaJI, AspA2I, AvrII, BinI, SatI, Fnu4HI, Fsp4HI, ItaI, BveI, Acc36I, BfuAI, BspMI, PfoI, FspBI, BfaI, MaeI, XspI, PfeI, TfiI, SsiI, AciI, BspACI, FaqI, BsiFI, BsmFI, HpyF3I, BstDEI, DdeI, SgsI, AscI, PalAI, CseI, HgaI, Lsp1109I, BbvI, BstV1I, CsiI, MabI, SexAI, PteI, BsePlm, BssHII, FokI, BstF5I, BtsCI, KflI, Sse1825I, SanDI, SaqAI, MseI, Tru1I, or Tru9I, or an isoschizomers of any of the aforesaid restriction enzymes. In some embodiments, the restriction enzyme is selected from AcuI, AiwI, AaR1, BaeI, BbsI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, Earl, EciI, Esp3I, FauI, FokI, HgaI, HphI, HpyAV, MbolI, MiyI, MnlI, Nme-AIII, PaqCI, PieI, SapI, SfaNI, AbsI, Acc65I, AgeI, AgsI, ArsI, AscI, AsiGI, AsiSI, Asp718I, AspA2I, AsuNHI, AvrII, Alw44I, AxyI, BamHI, BelI, BcnI, BgIII, Bpu1102I, BlnI, BmtI, BsaI, Bse21I, BshTI, BsmBI, Bso31I, BspOI, BspTNI, BstENI, Bsp119I, Bsp120I, Bsu15I, Bsu36I, CciNI, Cfr13I, Csp6I, Cfr9I, CspAI, Ec1136II, Eco31I, Eco53kI, Eco81I, Eco91I, Eco52I, EcoICRI, EcoNI, EcoRI, Esp3I, FseI, Hin6I, HindIII, HpaII, I-CeuI, I-PpoI, I-SceI, KpnI, Kpn2I, LmnI, LpnPI, MauBI, MspI, MluI, NcoI, NdeI, NheI, NotI, PalAI, PI-PspI, PI-SceI, PinAI, Psp124BI, RgaI, RigI, SacI, SalI, SbfI, SdaI, SfaAI, SgfI, SgsI, SmaI, SrfI, Sse83871, SstI, TaqI, TspMI, XagI, XbaI, XhoI, XmaI, XmaCI, XmaJI, MunI, HinfI, BspTI, Pfl23II, VspI, Bsp1407I, Psp1406I, Tru1I, Bpu10I, BcuI, PagI, BshTI, XmaJI, PfoI, SgsI, PteI, KflI, or an isoschizomer of any of the aforesaid restriction enzymes. In some embodiments, the restriction enzyme is selected from AbsI, Acc65I, AgeI, AgsI, ArsI, AscI, AsiGI, AsiSI, Asp718I, AspA2I, AsuNHI, AvrII, Alw44I, AxyI, BamHI, BelI, BcnI, BglII, Bpu1102I, BlnI, BmtI, BsaI, Bse21I, BshTI, BsmBI, Bso31I, BspOI, BspTNI, BstENI, Bsp119I, Bsp120I, Bsu15I, Bsu36I, CciNI, Cfr13I, Csp6I, Cfr9I, CspAI, Ec1136II, Eco31I, Eco53kI, Eco81I, Eco91I, Eco52I, EcoICRI, EcoNI, EcoRI, Esp3I, FseI, Hin6I, HindIII, HpaII, I-CeuI, I-PpoI, I-SceI, KpnI, Kpn2I, LmnI, LpnPI, MauBI, MspI, MluI, NcoI, NdeI, NheI, NotI, PalAI, PI-PspI, PI-SceI, PinAI, Psp124BI, RgaI, RigI, SacI, SalI, SbfI, SdaI, SfaAI, SgfI, SgsI, SmaI, SrfI, Sse83871, SstI, TaqI, TspMI, XagI, XbaI, XhoI, XmaI, XmaCI, XmaJI, MunI, HinfI, BspTI, Pfl23II, VspI, Bsp1407I, Psp1406I, Tru1I, Bpu10I, BcuI, PagI, BshTI, XmaJI, PfoI, SgsI, PteI, KflI, or an isoschizomer of any of the aforesaid restriction enzymes. In some embodiments, the restriction enzyme is AgeI, AvrII, BamHI, NheI, AscI, NotI, or an isoschizomer of any of the aforesaid restriction enzymes. In some embodiments, the restriction enzyme is AgeI, AvrII, BamHI, NheI, or an isoschizomer of any of the aforesaid restriction enzymes. In some embodiments, the restriction enzyme is AscI, NotI, or an isoschizomer of any of the aforesaid restriction enzymes. In some embodiments, the restriction enzyme site is a recognition site for a type IIS restriction enzyme. In some embodiments, the type IIS restriction enzyme is selected from, but not limited to, AcuI, AlwI, AaR1, BaeI, BbsI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, Earl, EciI, Esp3I, FauI, FokI, HgaI, HphI, HpyAV, MbolI, MlyI, MnlI, Nme-AIII, PaqCI, PleI, SapI, or SfaNI, or an isoschizomer of any of the aforesaid restriction enzymes. In some embodiments, each fragment of the plurality is devoid of recognition sites of a type IIS restriction enzyme. In some embodiments, each fragment of the plurality of fragments is devoid of recognition sites of a selected type IIS restriction enzyme. In some embodiments, each fragment of the plurality is devoid of recognition sites of two or more selected type IIS restriction enzymes. Additional non-limiting enzymes (e.g., type IIS restriction enzymes) include, for example, AasI, Acc36I, AccB7I, AceIII, AcpII, Adel, AfiI, AjuI, AlfI, AloI, Alw26I, AlwNI, AlwXI, AmaCSI, ApaBI, ApyPI, AquII, AquIII, AquIV, ArsI, Asp10HII, BasI, Bbr7I, BbvI, BbvII, Bbv16II, Bce4I, Bce83I, BceCI, BcgI, Bco5I, Bco116I, BcoKI, BflI, BfuAI, BglI, Bli736I, Bme585I, BpiI, BplI, BpmI, BpuAI, BpuJI, BpuSI, BsAJI, Bsc4I, Bsc91I, Bsc107I, BscAI, Bse3DI, BseGI, BseKI, BseLI, BseMI, BseMII, BseXI, BseZI, BsgI, BsiYI, BslI, BslFI, Bso31I, BsoMAI, Bsp24I, Bsp423I, BspBS31I, BspCNI, BspD6I, BsplS4I, BspKT5I, BspLU11III, BspMI, BspST5I, BspTNI, BspTS514I, BspWI, Bst6I, Bst12I, Bst19I, Bst71I, BstAPI, BstBS32I, BstFZ438I, BstGZ53I, BstHZ55I, BstlZ316I, BstMAI, BstMWI, BstOZ616I, BstPZ418I, BstTS5I, BstV1I, BstV2I, BstXI, CaiI, CjeI, CjePI, CspCI, CstMI, DraIII, DrdI, DseDI, Eam1104I, Eco31I, Eco5TI, EcoA4I, Eco57MI, EcoO44I, EcoP15I, Esp3I, Esp1396I, GsuI, HaeIV, Hin4I, Hpy178III, Hpy188III, HpyF1OVI, LweI, MmeI, MwoI, PfiBI, PfiMI, PhaI, PpiI, PsrI, RleAI, SdiI, SfiI, SmuI, Sth132I, StsI, TaqII, TspDTI, Tsp8EI, TspGWI, Tth111II, Van91I, or VpaK11AI, or an isoschizomer of any of the aforesaid restriction enzymes. In some embodiments, the restriction enzyme is capable of producing a 5′ overhang upon cleavage by the restriction enzyme. In some embodiments, cleavage by the restriction enzyme results in at least one or at least two (e.g., one or two) base pairs outside the 5′ overhang in its recognition site. In some embodiments, the restriction enzyme is capable of producing a 3′ overhang upon cleavage by the restriction enzyme. In some embodiments, the restriction enzyme is heat inactivatable. In some embodiments, the restriction enzyme recognizes a stretch of 4-8 base pairs, e.g., 4 base pairs, 5 base pairs, 6 base pairs, 7 base pairs, or 8 base pairs. In some embodiments, the restriction enzyme is not EcoRI.

In some embodiments, each fragment of the plurality is capable of ordered assembly with another fragment of the plurality based on complementarity of the unique 5′ overhang in one fragment of the plurality with the unique 3′ overhang in another fragment of the plurality, or the unique 3′ overhang in one fragment of the plurality with the unique 5′ overhang in another fragment of the plurality. In some embodiments, the unique 5′ overhang of one fragment of the plurality is complementary to the unique 3′ overhang of another fragment of the plurality. In some embodiments, the unique 3′ overhang of one fragment of the plurality is complementary to the unique 5′ overhang of another fragment of the plurality. In some embodiments, the unique 5′ overhang of one fragment of the plurality is partially complementary to the unique 3′ overhang of another fragment of the plurality. In some embodiments, the unique 5′ overhang of one fragment of the plurality is fully complementary to the unique 3′ overhang of another fragment of the plurality. In some embodiments, the unique 3′ overhang of one fragment of the plurality is partially complementary to the unique 5′ overhang of another fragment of the plurality. In some embodiments, the unique 3′ overhang of one fragment of the plurality is fully complementary to the unique 5′ overhang of another fragment of the plurality.

In some embodiments, the unique 5′ overhang and unique 3′ overhang of each fragment of the plurality results from cleavage of the fragment by a restriction enzyme, e.g., a type II restriction enzyme (e.g., a type IIS restriction enzyme). In some embodiments, the restriction enzyme is a type II restriction enzyme chosen from, but not limited to, Eco31I, BsaI Bso311, BspTNI, Esp3I, BpiI, Alw26I, BsmAI, BstMAI, BmsI, BhaI, BscAI, BscUI, BsmNI, BspST5I, Bst19I, CjeP338II, LweI, Mva131211, PhaI, SfaNI, LguI, BspQI, PsiSI, SapI, Alw44I, ApaLI, VneI, BamHI, BcnI, BglII, Bpu1102I, BlpI, Bsp1720I, CeIII, Bsp1191, AsuII, Bpu14I, BspT104I, BstBI, Csp45I, NspV, SfuI, Bsp120I, PspOMI, Bsu15I, BanIII, Bsa29I, BseCI, BshVI, BspDI, BspXI, BsuTUI, ClaI, Cfr10I, Bse118I, BsrFI, BssAI, Cfr13I, AspS9I, BmgT120I, PspPI, Sau96I, Csp6I, AfaI, CviQI, RsaNI, Eam11041, Bst6I, Earl, EcoO109I, DraII, EcoRI, Eco47I, AvaII, Bme18I, SinI, VpaK11BI, Eco52I, BseX3I, BstZI, EagI, EclXI, Eco81I, AxyI, Bse21I, Bsu36I, Eco88I, Ama87I, AvaI, BmeT1101, BsiHKCI, BsoBlm, Eco91I, BstEII, BstPI, EcoO65I, PspEI, Eco130I, BssT1I, EcoT14I, ErhI, StyI, HinlI, AcyI, BsaHI, BssNI, BstACI, Hsp92I, AspLEI, BstHHI, CfoI, HinPIlI, HspAI, HindIII, HpaII, BsiSI, HapII, Kpn2I, AccIII, Aor13HI, BseAI, Bsp13I, BspEI, MroI, MspI, BsiSI, HapII, MvaI, AjnI, BseBI, Bst2UI, BstNI, BstOI, Psp6I, PspGI, MluI, NcoI, Bsp19I, NdeI, FauNDI, NotI, CciNI, SalI, TaqI, XbaI, XhoI, BelI, CpoI, CspI, Rsr2I, RsrII, MunI, MfeI, Psp5II, PpuMI, PspPPI, Bsp143I, BfuCI, BssMI, BstKTI, BstMBI, DpnII, Kzo9I, NdeII, Sau3Aim, HinfI, MboI, BfuCI, BssMI, BstKTI, BstMBI, DpnII, Kzo9I, NdeII, Sau3Alm, BspTI, AfIII, BfrI, Bst98I, BstAFI, MspCI, Vha4641, Pfl23II, BsiWI, PspLI, Acc65I, Asp7181, VspI, AseI, PshBI, Bsp1407I, BsrGI, BstAUI, Psp1406I, AclI, NheI, AsuNHI, BmtI, Tru1I, MseI, Tru9I, BshNI, AccB1I, BanI, BspT107I, BseDI, BsaJI, BssECI, BfmI, BstSFI, SfcI, Bpu10I, BcuI, AhII, SpeI, PagI, CciI, BspHI, ReaI, TatI, XagI, BstENI, EcoNI, PsyI, AspI, PflFI, Tth111I, TasI, Sse9I, Tsp509I, TspEI, XapI, AcsI, ApoI, Bme1390I, BmrFI, BssKI, BstSCI, MspR9I, ScrFI, StyD4I, BseXI, BbvI, BstV1I, BshTI, AgeI, AsiGI, CspAI, PinAI, XmiI, AccI, FbII, NmuCI, Tsp45I, PsuI, BstX2I, BstYI, MflI, XhoII, XmaJI, AspA2I, AvrII, BinI, SatI, Fnu4HI, Fsp4HI, ItaI, BveI, Acc36I, BfuAI, BspMI, PfoI, FspBI, BfaI, MaeI, XspI, PfeI, TfiI, SsiI, AciI, BspACI, FaqI, BsiFI, BsmFI, HpyF3I, BstDEI, DdeI, SgsI, AscI, PalAI, CseI, HgaI, Lsp11091, BbvI, BstV1I, CsiI, MabI, SexAI, PteI, BsePlm, BssHII, FokI, BstF5I, BtsCI, KflI, Sse1825I, SanDI, SaqAI, MseI, Tru1I, or Tru9I, or an isoschizomers of any of the aforesaid restriction enzymes. In some embodiments, the restriction enzyme is selected from AcuI, AiwI, AaR1, BaeI, BbsI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, Earl, EciI, Esp3I, FauI, FokI, HgaI, HphI, HpyAV, MbolI, MlyI, MnlI, Nme-AIII, PaqCI, PleI, SapI, SfaNI, AbsI, Acc65I, AgeI, AgsI, ArsI, AscI, AsiGI, AsiSI, Asp718I, AspA2I, AsuNHI, AvrII, Alw44I, AxyI, BamHI, BelI, BcnI, BglII, Bpu11021, BlnI, BmtI, BsaI, Bse21I, BshTI, BsmBI, Bso31I, BspOI, BspTNI, BstENI, Bsp1191, Bsp120I, Bsu15I, Bsu36I, CciNI, Cfr13I, Csp6I, Cfr9I, CspAI, Ec1136II, Eco31I, Eco53kI, Eco81I, Eco91I, Eco52I, EcoICRI, EcoNI, EcoRI, Esp3I, FseI, Hin6I, HindIII, HpaII, I-CeuI, I-PpoI, I-SceI, KpnI, Kpn2I, LmnI, LpnPI, MauBI, MspI, MluI, NcoI, NdeI, NheI, NotI, PalAI, PI-PspI, PI-SceI, PinAI, Psp124BI, RgaI, RigI, SacI, SalI, SbfI, SdaI, SfaAI, SgfI, SgsI, SmaI, SrfI, Sse8387I, SstI, TaqI, TspMI, XagI, XbaI, XhoI, XmaI, XmaCI, XmaJI, MunI, HinfI, BspTI, Pfl23II, VspI, Bsp14071, Psp14061, Tru1I, Bpu101, BcuI, PagI, BshTI, XmaJI, PfoI, SgsI, PteI, KflI, or an isoschizomer of any of the aforesaid restriction enzymes. In some embodiments, the restriction enzyme is selected from AbsI, Acc65I, AgeI, AgsI, ArsI, AscI, AsiGI, AsiSI, Asp718I, AspA2I, AsuNHI, AvrII, Alw44I, AxyI, BamHI, BelI, BcnI, BglII, Bpu11021, BlnI, BmtI, BsaI, Bse21I, BshTI, BsmBI, Bso31I, BspOI, BspTNI, BstENI, Bsp1191, Bsp120I, Bsu15I, Bsu36I, CciNI, Cfr13I, Csp6I, Cfr9I, CspAI, Ec1136II, Eco31I, Eco53kI, Eco81I, Eco91I, Eco52I, EcoICRI, EcoNI, EcoRI, Esp3I, FseI, Hin6I, HindIII, HpaII, I-CeuI, I-PpoI, I-SceI, KpnI, Kpn2I, LmnI, LpnPI, MauBI, MspI, MluI, NcoI, NdeI, NheI, NotI, PalAI, PI-PspI, PI-SceI, PinAI, Psp124BI, RgaI, RigI, SacI, SalI, SbfI, SdaI, SfaAI, SgfI, SgsI, SmaI, SrfI, Sse8387I, SstI, TaqI, TspMI, XagI, XbaI, XhoI, XmaI, XmaCI, XmaJI, MunI, HinfI, BspTI, Pfl23II, VspI, Bsp1407I, Psp1406I, Tru1I, Bpu10I, BcuI, PagI, BshTI, XmaJI, PfoI, SgsI, PteI, KflI, or an isoschizomer of any of the aforesaid restriction enzymes. In some embodiments, the restriction enzyme is AgeI, AvrII, BamHI, NheI, AscI, NotI, or an isoschizomer of any of the aforesaid restriction enzymes. In some embodiments, the restriction enzyme is AgeI, AvrII, BamHI, NheI, or an isoschizomer of any of the aforesaid restriction enzymes. In some embodiments, the restriction enzyme is AscI, NotI, or an isoschizomer of any of the aforesaid restriction enzymes. In some embodiments, the unique 5′ overhang and unique 3′ overhang of each fragment of the plurality results from cleavage of the fragment by a selected type IIS restriction enzyme. In some embodiments, the type IIS restriction enzyme is selected from, but not limited to, AcuI, AlwI, AaR1, BaeI, BbsI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, Earl, EciI, Esp3I, FauI, FokI, HgaI, HphI, HpyAV, MbolI, MlyI, MnlI, Nme-AIII, PaqCI, PleI, SapI, or SfaNI, or an isoschizomer of any of the aforesaid restriction enzymes. In some embodiments, the type IIS restriction enzyme is BsaI. In some embodiments, the type IIS restriction enzyme is BsmBI. Additional non-limiting enzymes (e.g., type IIS restriction enzymes) suitable for use include, for example, AasI, Acc36I, AccB7I, AceIII, AcpII, Adel, AfiI, AjuI, AlfI, AloI, Alw26I, AlwNI, AlwXI, AmaCSI, ApaBI, ApyPI, AquII, AquIII, AquIV, ArsI, Asp10HII, BasI, Bbr7I, BbvI, BbvII, Bbv16II, Bce4I, Bce83I, BceCI, BcgI, Bco5I, Bco116I, BcoKI, BflI, BfuAI, BglI, Bli736I, Bme585I, BpiI, BplI, BpmI, BpuAI, BpuJI, BpuSI, BsAJI, Bsc4I, Bsc91I, Bsc107I, BscAI, Bse3DI, BseGI, BseKI, BseLI, BseMI, BseMII, BseXI, BseZI, BsgI, BsiYI, BslI, BslFI, Bso31I, BsoMAI, Bsp24I, Bsp423I, BspBS31I, BspCNI, BspD6I, BsplS4I, BspKT5I, BspLU11111, BspMI, BspST5I, BspTNI, BspTS514I, BspWI, Bst6I, Bst12I, Bst19I, Bst71I, BstAPI, BstBS32I, BstFZ438I, BstGZ53I, BstHZ55I, BstlZ316I, BstMAI, BstMWI, BstOZ616I, BstPZ418I, BstTS5I, BstV1I, BstV2I, BstXI, CaiI, CjeI, CjePI, CspCI, CstMI, DraIII, DrdI, DseDI, Eam11041, Eco31I, Eco5TI, EcoA4I, Eco57MI, EcoO44I, EcoP15I, Esp3I, Esp13961, GsuI, HaeIV, Hin4I, Hpy178III, Hpy188III, HpyF1OVI, LweI, MmeI, MwoI, PfiBI, PfiMI, PhaI, PpiI, PsrI, RleAI, SdiI, SfiI, SmuI, Sth1321, StsI, TaqII, TspDTI, Tsp8EI, TspGWI, Tth1111I, Van91I, or VpaK11AI, or an isoschizomer of any of the aforesaid restriction enzymes. In some embodiments, the restriction enzyme recognizes a discontinuous site, for example, AasI, AccB7I, AcpII, Adel, AfiI, AjuI, AlwNI, ApaBI, ArsI, Asp10HII, BaeI, BasI, Bce4I, BceCI, BcgI, BfiI, BglI, Bsc4I, Bsc107I, BseLI, BsiYI, BslI, BspWI, BstAPI, BstH255I, BstlZ316I, BstMWI, BstXI, CaiI, BraIII, DrdI, DseDI, HpyF10VI, PflBI, PflMI, SdiI, SfiI, Tsp8EI, or Van91I, or an isoschizomer of any of the aforesaid restriction enzymes. In some embodiments, the restriction enzyme is capable of producing a 5′ overhang upon cleavage by the restriction enzyme. In some embodiments, cleavage by the restriction enzyme results in at least one or at least two (e.g., one or two) base pairs outside the 5′ overhang in its recognition site. In some embodiments, the restriction enzyme is capable of producing a 3′ overhang upon cleavage by the restriction enzyme. In some embodiments, the restriction enzyme is heat inactivatable. In some embodiments, the restriction enzyme recognizes a stretch of 4-8 base pairs, e.g., 4 base pairs, 5 base pairs, 6 base pairs, 7 base pairs, or 8 base pairs. In some embodiments, the restriction enzyme is not EcoRI.

In some embodiments, the fragments of a plurality of subgenomic fragments result from cleavage with a first restriction enzyme, e.g., a selected first type IIS restriction enzyme; and/or (b) the fragments of a plurality of subfragments result from cleavage with a second restriction enzyme, e.g., a selected second type IIS restriction enzyme, wherein the first restriction enzyme, e.g., a selected first type IIS restriction enzyme, is different from the second restriction enzyme, e.g., a selected second type IIS restriction enzyme. In some embodiments, the first restriction enzyme is BsaI, and the second restriction enzyme is BsmBI. In some embodiments, the first restriction enzyme is BsmBI, and the second restriction enzyme is BsaI.

In some embodiments, each fragment (e.g., subgenomic fragment or subfragment described herein) of the plurality comprises a unique 5′ overhang and a unique 3′ overhang, e.g., a unique 5′ overhang and unique 3′ overhang described herein.

In some embodiments, each fragment of the plurality is capable of ordered assembly with another fragment of the plurality based on the overlap in nucleotide sequence at the 5′ end of one fragment (e.g., a baculovirus genome sequence) with nucleotide sequence at the 3′ end of another fragment (e.g., a baculovirus genome sequence). In some embodiments, the overlap in nucleotide sequence between two fragments is 4-50 (e.g., 4-45, 4-40, 4-35, 4-30, 4-25, 4-20, 4-15, 4-10, 10-50, 10-45, 10-40, 10-35, 10-30, 10-25, 10-20, 10-15, 15-50, 15-45, 15-40, 15-35, 15-30, 15-25, 15-20, 20-50, 20-45, 20-40, 20-35, 20-30, 20-25, 25-50, 25-45, 25-40, 25-35, 25-30, 30-50, 30-45, 30-40, 30-35, 35-50, 35-45, 35-40, 40-50, 40-45) base pairs, e.g., contiguous base pairs. In some embodiments, following cleavage by a selected type II restriction enzyme disclosed herein, a 5′ exonuclease creates a single-stranded region of complementarity corresponding to the overlapping nucleotide sequence between fragments, producing fragments capable of annealing with each other. In some embodiments, gaps between the annealed fragments are filled in by a DNA polymerase.

In some embodiments, the plurality of fragments is a plurality of subfragments described herein. In some embodiments, the plurality of fragments is a plurality of subgenomic fragments described herein.

In some embodiments, each fragment of the plurality of subgenomic fragments is present in a separate first carrier vector. In some embodiments, each fragment of a plurality of subfragments is present in a separate second carrier vector. In some embodiments, each of the separate first carrier vectors are maintained (e.g., stored) separately from each other. In some embodiments, each of the separate second carrier vectors are maintained (e.g., stored) separately from each other.

In some embodiments, a plurality of subfragments constitute the nucleotide sequence of one subgenomic fragment. In some embodiments, a plurality of subfragments constitute the nucleotide sequence of two or more subgenomic fragments (e.g., 2-20, 5-20, 10-20, 12-20, 14-18, 14, 15, 16, 17, or 18 subgenomic fragments).

In some embodiments, a plurality of subgenomic fragments (e.g., 2-20, 5-20, 10-20, 12-20, 14-18, 14, 15, 16, 17, or 18 subgenomic fragments) constitute the nucleotide sequence of a baculovirus genome (e.g., variant baculovirus genome) or baculovirus expression construct described herein.

In some embodiments, one or more of the fragments of the plurality comprise a nucleotide sequence of interest. In some embodiments, the sequence of interest encodes a polypeptide of interest or nucleic acid of interest. In some embodiments, the polypeptide or nucleic acid of interest is a therapeutic polypeptide (e.g., therapeutic protein) or a therapeutic nucleotide sequence (e.g., a therapeutic RNAi). In some embodiments, the polypeptide of interest is a detectable marker gene, such as green fluorescent protein (GFP) or secreted embryonic alkaline phosphatase (SEAP). In some embodiments, the sequence of interest comprises one or more adeno-associated viral (AAV) gene encoding regions and/or a payload. In some embodiments, the sequence of interest is modified to remove one or more or all recognition sites of the restriction enzyme, e.g., a selected type II restriction enzyme (e.g., a selected type IIS restriction enzyme) used to generate the fragments (e.g., subgenomic fragments or subfragments) of the plurality. In some embodiments, the nucleotide sequence of interest is chemically synthesized. In some embodiments, the nucleotide sequence of interest is generated by a PCR-based method. In some embodiments, the nucleotide sequence of interest sequence is generated by a combination of chemical synthesis and a PCR-based method. In some embodiments, the nucleotide sequence of interest is a non-templated nucleotide sequence of interest (e.g., non-templated fragment). In some embodiments, the nucleotide sequence of interest is inserted into one or more fragments of the plurality using available restriction enzyme sites. In some embodiments, the nucleotide sequence of interest is inserted into one or more fragments of the plurality using Gibson Assembly™

The nucleotide sequence of fragments of the plurality may be generated using various conventional cloning or synthetic methods. For example, all or a portion of a fragment (e.g., subfragment or subgenomic fragment) can be generated using chemical synthesis. In some embodiments, all or a portion of a fragment is generated using a PCR-based method. In some embodiments, the fragment is a non-templated fragment). In some embodiments, all or a portion of a fragment is generated using a restriction enzyme-based method, e.g., obtaining a portion or all of a fragment by “cutting out” a sequence of interest from a source nucleotide sequence (e.g., a vector comprising the sequence of interest). In some embodiments, a fragment may be prepared using a combination of one or more of the aforementioned methods (or any other suitable method known in the art).

### Baculovirus Genome and Vectors

Provided herein are baculovirus genomes, e.g., variant baculovirus genomes, and vectors comprising the same (e.g., destination vectors such as bacterial artificial chromosomes (BACs)), which comprise fewer restriction enzyme sites relative to a reference baculovirus genome described herein, e.g., a wild-type baculovirus genome described herein. In some embodiments, the variant baculovirus genome comprises fewer recognition sites for type IIS restriction enzymes relative to a reference baculovirus genome. In some embodiments, the variant baculovirus genome comprises fewer recognition sites for a selected type II restriction enzyme (e.g., selected type IIS restriction enzyme), relative to a reference baculovirus genome, e.g., a wild-type baculovirus genome. In some embodiments, the variant baculovirus genome comprises fewer recognition sites for two or more (e.g., 2, 3, 4, 5, 6, 7, 8, 9, 10, or more) selected type II restriction enzymes, e.g., selected type IIS restriction enzymes) relative to a reference baculovirus genome, e.g., a wild-type baculovirus genome.

In some embodiments, the variant baculovirus genome comprises at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 56, at least 57, at least 58, at least 59, or at least 60 fewer restriction enzyme sites (e.g., functional naturally occurring restriction enzyme sites), e.g., type II restriction enzyme sites (e.g., type IIS restriction enzyme sites), relative to the nucleotide sequence in a reference baculovirus genome. In some embodiments, the baculovirus expression construct comprises 1-60, 2-60, 4-60, 6-60, 8-60, 10-60, 15-60, 20-60, 25-60, 30-60, 35-60, 40-60, 45-60, 50-60, 55-60, 1-50, 2-50, 4-50, 6-50, 8-50, 10-50, 15-50, 20-50, 25-50, 30-50, 35-50, 40-50, 45-50, 1-40, 2-40, 4-40, 6-40, 8-40, 10-40, 15-40, 20-40, 25-40, 30-40, 35-40, 1-30, 2-30, 4-30, 6-30, 8-30, 10-30, 15-30, 20-30, 25-30, 1-20, 2-20, 4-20, 6-20, 8-20, 10-20, 15-20, 1-10, 2-10, 4-10, 6-10, or 8-10 fewer restriction enzyme sites (functional naturally occurring restriction enzyme sites), e.g., type II restriction enzyme sites (e.g., type IIS restriction enzyme sites), relative to the nucleotide sequence in a reference baculovirus genome.

In some embodiments, the variant baculovirus genome is devoid of recognition sites for a selected restriction enzyme. In some embodiments, the variant baculovirus genome is devoid of recognition sites for two or more selected restriction enzymes. In some embodiments, the variant baculovirus genome is devoid of recognition sites (e.g. naturally occurring recognition sites) for a selected type II restriction enzyme. In some embodiments, the variant baculovirus genome is devoid of recognition sites (e.g., naturally occurring recognition sties) for two or more selected type II restriction enzymes. In some embodiments, the restriction enzyme is a type II restriction enzyme chosen from, but not limited to, Eco31I, BsaI Bso311, BspTNI, Esp3I, BpiI, Alw26I, BsmAI, BstMAI, BmsI, BhaI, BscAI, BscUI, BsmNI, BspST5I, Bst19I, CjeP338II, LweI, Mva1312II, PhaI, SfaNI, LguI, BspQI, PsiSI, SapI, Alw44I, ApaLI, VneI, BamHI, BcnI, BglII, Bpu1102I, BlpI, Bsp1720I, CeIII, Bsp1191, AsuII, Bpu14I, BspT104I, BstBI, Csp45I, NspV, SfuI, Bsp120I, PspOMI, Bsu15I, BanIII, Bsa29I, BseCI, BshVI, BspDI, BspXI, BsuTUI, ClaI, Cfr10I, Bse118I, BsrFI, BssAI, Cfr13I, AspS9I, BmgT120I, PspPI, Sau96I, Csp6I, AfaI, CviQI, RsaNI, Eam1104I, Bst6I, Earl, EcoO109I, DraII, EcoRI, Eco47I, AvaII, Bme18I, SinI, VpaK11BI, Eco52I, BseX3I, BstZI, EagI, EclXI, Eco81I, AxyI, Bse21I, Bsu36I, Eco88I, Ama87I, AvaI, BmeT1101, BsiHKCI, BsoBlm, Eco91I, BstEII, BstPI, EcoO65I, PspEI, Eco130I, BssT1I, EcoT14I, ErhI, StyI, HinlI, AcyI, BsaHI, BssNI, BstACI, Hsp92I, AspLEI, BstHHI, CfoI, HinPIlI, HspAI, HindIII, HpaII, BsiSI, HapII, Kpn2I, AccIII, Aor13HI, BseAI, Bsp13I, BspEI, MroI, MspI, BsiSI, HapII, MvaI, AjnI, BseBI, Bst2UI, BstNI, BstOI, Psp6I, PspGI, MluI, NcoI, Bsp19I, NdeI, FauNDI, NotI, CciNI, SalI, TaqI, XbaI, XhoI, BelI, CpoI, CspI, Rsr2I, RsrII, MunI, MfeI, Psp5II, PpuMI, PspPPI, Bsp143I, BfuCI, BssMI, BstKTI, BstMBI, DpnII, Kzo9I, NdeII, Sau3Aim, HinfI, MboI, BfuCI, BssMI, BstKTI, BstMBI, DpnII, Kzo9I, NdeII, Sau3Alm, BspTI, AfIII, BfrI, Bst98I, BstAFI, MspCI, Vha4641, Pfl23II, BsiWI, PspLI, Acc65I, Asp718I, VspI, AseI, PshBI, Bsp1407I, BsrGI, BstAUI, Psp1406I, AclI, NheI, AsuNHI, BmtI, Tru1I, MseI, Tru9I, BshNI, AccB1I, BanI, BspT107I, BseDI, BsaJI, BssECI, BfmI, BstSFI, SfcI, Bpu10I, BcuI, AhII, SpeI, PagI, CciI, BspHI, ReaI, TatI, XagI, BstENI, EcoNI, PsyI, AspI, PflFI, Tth111I, TasI, Sse9I, Tsp509I, TspEI, XapI, AcsI, ApoI, Bme1390I, BmrFI, BssKI, BstSCI, MspR9I, ScrFI, StyD4I, BseXI, BbvI, BstV1I, BshTI, AgeI, AsiGI, CspAI, PinAI, XmiI, AccI, FbII, NmuCI, Tsp45I, PsuI, BstX2I, BstYI, MflI, XhoII, XmaJI, AspA2I, AvrII, BinI, SatI, Fnu4HI, Fsp4HI, ItaI, BveI, Acc36I, BfuAI, BspMI, PfoI, FspBI, BfaI, MaeI, XspI, PfeI, TfiI, SsiI, AciI, BspACI, FaqI, BsiFI, BsmFI, HpyF3I, BstDEI, DdeI, SgsI, AscI, PalAI, CseI, HgaI, Lsp1109I, BbvI, BstV1I, CsiI, MabI, SexAI, PteI, BsePlm, BssHII, FokI, BstF5I, BtsCI, KflI, Sse1825I, SanDI, SaqAI, MseI, Tru1I, or Tru9I, or an isoschizomers of any of the aforesaid restriction enzymes. In some embodiments, the restriction enzyme is selected from AcuI, AlwI, AaR1, BaeI, BbsI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, Earl, EciI, Esp3I, FauI, FokI, HgaI, HphI, HpyAV, MbolI, MlyI, MnlI, Nme-AIII, PaqCI, PleI, SapI, SfaNI, AbsI, Acc65I, AgeI, AgsI, ArsI, AscI, AsiGI, AsiSI, Asp718I, AspA2I, AsuNHI, AvrII, Alw44I, AxyI, BamHI, BelI, BcnI, BglII, Bpu1102I, BlnI, BmtI, BsaI, Bse21I, BshTI, BsmBI, Bso31I, BspOI, BspTNI, BstENI, Bsp119I, Bsp120I, Bsu15I, Bsu36I, CciNI, Cfr13I, Csp6I, Cfr9I, CspAI, Ec1136II, Eco31I, Eco53kI, Eco81I, Eco91I, Eco52I, EcoICRI, EcoNI, EcoRI, Esp3I, FseI, Hin6I, HindIII, HpaII, I-CeuI, I-PpoI, I-SceI, KpnI, Kpn2I, LmnI, LpnPI, MauBI, MspI, MluI, NcoI, NdeI, NheI, NotI, PalAI, PI-PspI, PI-SceI, PinAI, Psp124BI, RgaI, RigI, SacI, SalI, SbfI, SdaI, SfaAI, SgfI, SgsI, SmaI, SrfI, Sse83871, SstI, TaqI, TspMI, XagI, XbaI, XhoI, XmaI, XmaCI, XmaJI, MunI, HinfI, BspTI, Pfl23II, VspI, Bsp1407I, Psp1406I, Tru1I, Bpu10I, BcuI, PagI, BshTI, XmaJI, PfoI, SgsI, PteI, KflI, or an isoschizomer of any of the aforesaid restriction enzymes. In some embodiments, the restriction enzyme is selected from AbsI, Acc65I, AgeI, AgsI, ArsI, AscI, AsiGI, AsiSI, Asp718I, AspA2I, AsuNHI, AvrII, Alw44I, AxyI, BamHI, BelI, BcnI, BglII, Bpu1102I, BlnI, BmtI, BsaI, Bse21I, BshTI, BsmBI, Bso31I, BspOI, BspTNI, BstENI, Bsp119I, Bsp120I, Bsu15I, Bsu36I, CciNI, Cfr13I, Csp6I, Cfr9I, CspAI, Ec1136II, Eco31I, Eco53kI, Eco81I, Eco91I, Eco52I, EcoICRI, EcoNI, EcoRI, Esp3I, FseI, Hin6I, HindIII, HpaII, I-CeuI, I-PpoI, I-SceI, KpnI, Kpn2I, LmnI, LpnPI, MauBI, MspI, MluI, NcoI, NdeI, NheI, NotI, PalAI, PI-PspI, PI-SceI, PinAI, Psp124BI, RgaI, RigI, SacI, SalI, SbfI, SdaI, SfaAI, SgfI, SgsI, SmaI, SrfI, Sse83871, SstI, TaqI, TspMI, XagI, XbaI, XhoI, XmaI, XmaCI, XmaJI, MunI, HinfI, BspTI, Pfl23II, VspI, Bsp1407I, Psp1406I, Tru1I, Bpu10I, BcuI, PagI, BshTI, XmaJI, PfoI, SgsI, PteI, KflI, or an isoschizomer of any of the aforesaid restriction enzymes. In some embodiments, the restriction enzyme is AgeI, AvrII, BamHI, NheI, AscI, NotI, or an isoschizomer of any of the aforesaid restriction enzymes. In some embodiments, the restriction enzyme is AgeI, AvrII, BamHI, NheI, or an isoschizomer of any of the aforesaid restriction enzymes. In some embodiments, the restriction enzyme is AscI, NotI, or an isoschizomer of any of the aforesaid restriction enzymes. In some embodiments, the variant baculovirus genome is devoid of recognition sites for a selected type IIS restriction enzyme. In some embodiments, the variant baculovirus genome is devoid of recognition sites for two or more selected type IIS restriction enzymes. In some embodiments, the type IIS restriction enzyme or two or more type IIS restriction enzymes are selected from, but not limited to, AcuI, AlwI, AaR1, BaeI, BbsI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, Earl, EciI, Esp3I, FauI, FokI, HgaI, HphI, HpyAV, MbolI, MlyI, MnlI, Nme-AIII, PaqCI, PleI, SapI, or SfaNI, or an isoschizomer of any of the aforesaid restriction enzymes. In some embodiments, the type IIS restriction enzyme site is a BsaI restriction enzyme site. In some embodiments, the type IIS restriction enzyme site is a BsmBI restriction enzyme site. In some embodiments, the type IIS restriction enzyme site is a BsaI restriction enzyme site. In some embodiments, the type IIS restriction enzyme site is a combination of BsaI and BsmBI restriction enzyme sites. In some embodiments, the variant baculovirus genome comprises no functional type IIS restriction enzyme sites. Additional non-limiting restriction enzymes (e.g., type IIS restriction enzymes) include, for example, AasI, Acc36I, AccB7I, AceIII, AcpII, Adel, AfiI, AjuI, AlfI, AloI, Alw26I, AlwNI, AlwXI, AmaCSI, ApaBI, ApyPI, AquII, AquIII, AquIV, ArsI, Asp10HII, BasI, Bbr7I, BbvI, BbvII, Bbv16II, Bce4I, Bce83I, BceCI, BcgI, Bco5I, Bco116I, BcoKI, BflI, BfuAI, BglI, Bli736I, Bme585I, BpiI, BplI, BpmI, BpuAI, BpuJI, BpuSI, BsAJI, Bsc4I, Bsc91I, Bsc107I, BscAI, Bse3DI, BseGI, BseKI, BseLI, BseMI, BseMII, BseXI, BseZI, BsgI, BsiYI, BslI, BslFI, Bso31I, BsoMAI, Bsp24I, Bsp423I, BspBS31I, BspCNI, BspD6I, BsplS4I, BspKT5I, BspLU111I, BspMI, BspST5I, BspTNI, BspTS514I, BspWI, Bst6I, Bst12I, Bst19I, Bst71I, BstAPI, BstBS32I, BstFZ438I, BstGZ53I, BstHZ55I, BstlZ316I, BstMAI, BstMWI, BstOZ616I, BstPZ418I, BstTS5I, BstV1I, BstV2I, BstXI, CaiI, CjeI, CjePI, CspCI, CstMI, DraIII, DrdI, DseDI, Eam11041, Eco31I, Eco5TI, EcoA4I, Eco57MI, EcoO44I, EcoP15I, Esp3I, Esp13961, GsuI, HaeIV, Hin4I, Hpy178III, Hpy188III, HpyF1OVI, LweI, MmeI, MwoI, PfiBI, PfiMI, PhaI, PpiI, PsrI, RleAI, SdiI, SfiI, SmuI, Sth1321, StsI, TaqII, TspDTI, Tsp8EI, TspGWI, Tth111II, Van91I, or VpaK11AI, or an isoschizomer of any of the aforesaid restriction enzymes. In some embodiments, the restriction enzyme is capable of producing a 5′ overhang upon cleavage by the restriction enzyme. In some embodiments, cleavage by the restriction enzyme results in at least one or at least two (e.g., one or two) base pairs outside the 5′ overhang in its recognition site. In some embodiments, the restriction enzyme is capable of producing a 3′ overhang upon cleavage by the restriction enzyme. In some embodiments, the restriction enzyme is heat inactivatable. In some embodiments, the restriction enzyme recognizes a stretch of 4-8 base pairs, e.g., 4 base pairs, 5 base pairs, 6 base pairs, 7 base pairs, or 8 base pairs. In some embodiments, the restriction enzyme is not EcoRI.

In some embodiments, the fewer (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 56, at least 57, at least 58, at least 59, or at least 60, 1-60, 2-60, 4-60, 6-60, 8-60, 10-60, 15-60, 20-60, 25-60, 30-60, 35-60, 40-60, 45-60, 50-60, 55-60, 1-50, 2-50, 4-50, 6-50, 8-50, 10-50, 15-50, 20-50, 25-50, 30-50, 35-50, 40-50, 45-50, 1-40, 2-40, 4-40, 6-40, 8-40, 10-40, 15-40, 20-40, 25-40, 30-40, 35-40, 1-30, 2-30, 4-30, 6-30, 8-30, 10-30, 15-30, 20-30, 25-30, 1-20, 2-20, 4-20, 6-20, 8-20, 10-20, 15-20, 1-10, 2-10, 4-10, 6-10, or 8-10 fewer) functional type II restriction enzyme sites (e.g., type IIS restriction enzyme sites) in the variant baculovirus genome relative to the reference baculovirus genome are the same type II restriction enzyme site (e.g., same type IIS restriction enzyme site).

In some embodiments, the fewer (e.g., at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 56, at least 57, at least 58, at least 59, or at least 60, 1-60, 2-60, 4-60, 6-60, 8-60, 10-60, 15-60, 20-60, 25-60, 30-60, 35-60, 40-60, 45-60, 50-60, 55-60, 1-50, 2-50, 4-50, 6-50, 8-50, 10-50, 15-50, 20-50, 25-50, 30-50, 35-50, 40-50, 45-50, 1-40, 2-40, 4-40, 6-40, 8-40, 10-40, 15-40, 20-40, 25-40, 30-40, 35-40, 1-30, 2-30, 4-30, 6-30, 8-30, 10-30, 15-30, 20-30, 25-30, 1-20, 2-20, 4-20, 6-20, 8-20, 10-20, 15-20, 1-10, 2-10, 4-10, 6-10, or 8-10 fewer) functional type II restriction enzyme site (e.g., type IIS restriction enzyme sites) in the variant baculovirus genome relative to the reference baculovirus genome are different type IIS restriction enzyme sites, e.g., a combination of first type II restriction enzyme sites and second type II restriction enzyme sites (e.g., a combination of first type IIS restriction enzyme sites and second type IIS restriction enzyme sites). In some embodiments, the fewer functional type II restriction enzyme sites (e.g., type IIS restriction enzyme sites) in the variant baculovirus genome relative to the reference baculovirus genome are restriction enzyme sites for 2, 3, 4, 5, 6, 7, 8, 9, 10, or more type II restriction enzymes (e.g., type IIS restriction enzymes).

In some embodiments, (i) the first type IIS restriction enzyme site is selected from a type II restriction enzyme site described herein, and (ii) the second type IIS restriction enzyme site is selected from a type II restriction enzyme site described herein, wherein the first selected type II restriction enzyme site and second selected type II restriction enzyme site are different.

In some embodiments, (i) the first type IIS restriction enzyme site is selected from an AcuI, AlwI, AaR1, BaeI, BbsI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, Earl, EciI, Esp3I, FauI, FokI, HgaI, HphI, HpyAV, MbolI, MlyI, MnlI, Nme-AIII, PaqCI, PleI, SapI, or SfaNI restriction enzyme site, or a restriction enzyme site of an isoschizomer of any one of the preceding enzymes; and (ii) the second type IIS restriction enzyme site is selected from an AcuI, AlwI, AaR1, BaeI, BbsI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, Earl, EciI, Esp3I, FauI, FokI, HgaI, HphI, HpyAV, MbolI, MlyI, MnlI, Nme-AIII, PaqCI, PleI, SapI, or SfaNI restriction enzyme site, or a restriction enzyme site of an isoschizomer of any one of the preceding enzymes; wherein the first type IIS restriction enzyme site and the second type IIS restriction enzyme site are different. In some embodiments, the first type IIS restriction enzyme site is a BsaI restriction enzyme site and the second type IIS restriction enzyme site is a BsmBI restriction enzyme site. Additional non-limiting restriction enzymes (e.g., type IIS restriction enzymes) include, for example, AasI, Acc36I, AccB7I, AceIII, AcpII, Adel, AfiI, AjuI, AlfI, AloI, Alw26I, AlwNI, AlwXI, AmaCSI, ApaBI, ApyPI, AquII, AquIII, AquIV, ArsI, Asp10HII, BasI, Bbr7I, BbvI, BbvII, Bbv16II, Bce4I, Bce83I, BceCI, BcgI, Bco5I, Bco116I, BcoKI, BflI, BfuAI, BglI, Bli736I, Bme585I, BpiI, BplI, BpmI, BpuAI, BpuJI, BpuSI, BsAJI, Bsc4I, Bsc91I, Bsc107I, BscAI, Bse3DI, BseGI, BseKI, BseLI, BseMI, BseMII, BseXI, BseZI, BsgI, BsiYI, BslI, BslFI, Bso311, BsoMAI, Bsp24I, Bsp4231, BspBS311, BspCNI, BspD6I, BsplS4I, BspKT5I, BspLU11III, BspMI, BspST5I, BspTNI, BspTS514I, BspWI, Bst6I, Bst12I, Bst19I, Bst71I, BstAPI, BstBS32I, BstFZ438I, BstGZ53I, BstHZ55I, BstlZ316I, BstMAI, BstMWI, BstOZ616I, BstPZ418I, BstTS5I, BstV1I, BstV2I, BstXI, CaiI, CjeI, CjePI, CspCI, CstMI, DraIII, DrdI, DseDI, Eam1104I, Eco31I, Eco5TI, EcoA4I, Eco57MI, EcoO44I, EcoP15I, Esp3I, Esp1396I, GsuI, HaeIV, Hin4I, Hpy178III, Hpy188III, HpyF1OVI, LweI, MmeI, MwoI, PfiBI, PfiMI, PhaI, PpiI, PsrI, RleAI, SdiI, SfiI, SmuI, Sth132I, StsI, TaqII, TspDTI, Tsp8EI, TspGWI, Tth111II, Van91I, or VpaK11AI, or an isoschizomer of any of the aforesaid restriction enzymes.

In some embodiments, the variant baculovirus genome comprises at least 3, 4, or 5-fold fewer functional first type IIS restriction enzyme sites (e.g., BsaI restriction enzyme sites), relative to a second type IIS restriction enzyme site (e.g., BsmBI restriction enzyme sites). In some embodiments, the variant baculovirus genome comprises at least 3, 4, or 5-fold more functional second type IIS restriction enzyme sites (e.g., BsmBI type II restriction enzyme sites) relative to a first type IIS restriction enzyme site (e.g., BsaI type II restriction enzyme sites).

In some embodiments, the variant baculovirus genome comprises: (i) at least 5-15 (e.g., 5-12, 5-13, 5-14, 5-15, 5-10, 10-12, 10-13, 10-14, 11-15, 11-15, 11-13, 12-15, 12-14, 10, 11, 12, 13, 14, or 15) fewer functional BsaI type II restriction enzyme sites relative to a reference baculovirus genome (e.g., AcMNPV genome); and/or (ii) at least 40-50 (e.g., 40-45, 41-50, 41-45, 42-50, 42-45, 43-50, 43-45, 44-50, 45-50, 46-50, 47-50, 48-50, 40, 41, 42, 43, 44, 45, 46, 47, 48, 49, or 50) fewer functional BsmBI type II restriction enzyme sites relative to a reference baculovirus genome (e.g., AcMNPV genome).

In some embodiments, the variant baculovirus genome comprises 11 fewer functional BsaI type II restriction enzyme sites and/or 45 fewer functional BsmBI type II restriction enzyme sites relative to a reference baculovirus genome (e.g., AcMNPV genome).

In some embodiments, the type II restriction enzyme site (e.g., type IIS restriction enzyme site) comprises a modification, e.g., an insertion, deletion, or substitution, that renders the site inactive or incapable of being recognized by the corresponding type II restriction enzyme (e.g., type IIS restriction enzyme). In some embodiments, the type II restriction enzyme site (e.g., type IIS restriction enzyme site) comprises one, two, three, four, five or six modifications e.g., insertions, deletions, or substitutions, relative to the corresponding functional type II restriction enzyme site (e.g., type IIS restriction enzyme site). In some embodiments, the type II restriction enzyme site (e.g., type IIS restriction enzyme site) comprises one, two, three, four, five or six different nucleotides relative to the corresponding functional type II restriction enzyme site (e.g., type IIS restriction enzyme site). In some embodiments, the type II restriction enzyme site (e.g., type IIS restriction enzyme site) is absent or deleted.

In some embodiments, the type II restriction enzyme site (e.g., type IIS restriction enzyme site) comprises a modification, e.g., substitution, wherein the modification comprises a nucleotide from a different naturally occurring baculovirus genome sequence (e.g., a reference baculovirus genome of the same genus). In some embodiments, the type II restriction enzyme site (e.g., type IIS restriction enzyme site) comprises a different nucleotide relative to the corresponding functional type IIS restriction enzyme site, wherein the different nucleotide is from a different naturally occurring baculovirus genome sequence.

Wild-type baculovirus genomes which can serve as reference baculovirus genomes are well known in the art (see, e.g., www.talk.ictvonline.org/ictv-reports/ictv_online_report/dsdna-viruses/w/baculoviridae, the contents of which are herein incorporated by reference in their entirety).

In some embodiments, the reference baculovirus genome is the genome of an alpha-baculovirus, beta-baculovirus, delta-baculovirus, or gamma-baculovirus. In some embodiments, the reference baculovirus genome is an alpha-baculovirus genome. In some embodiments, the reference baculovirus genome is beta-baculovirus genome. In some embodiments, the reference baculovirus genome is an delta-baculovirus genome. In some embodiments, the reference baculovirus genome is a gamma-baculovirus genome. In some embodiments, the reference baculovirus genome is the genome of an alpha-baculovirus selected from: Adoxophyes honmai nucleopolyhedrovirus (NPV), Agrotis ipsilon multiple nucleopolyhedrovirus (MNPV), Agrotis segetum NPVA, Agrotis segetum NPVB, Antheraea pernyi NPV, Anticarsia gemmatalis MNPV, Autographa californica MNPV (AcMNPV), Bombyx mori NPV (BmNPV), Buzura suppressaria NPV, Catopsilia pomona NPV, Choristoneura fumiferana DEF MNPV, Choristoneura fumiferana MNPV, Choristoneura murinana NPV, Choristoneura rosaceana NPV, Chrysodeixis chalcites NPV, Chrysodeixis includens NPV, Clanis bilineata NPV, Condylorrhiza vestigialis NPV, Cryptophlebia peltastica NPV, Cyclophragma undans NPV, Ectropis obliqua NPV, Epiphyas postvittana NPV, Euproctis pseudoconspersa NPV, Helicoverpa armigera NPV, Hemileuca species NPV, Hyphantria cunea NPV, Hyposidra talaca NPV, Lambdina fiscellaria NPV, Leucania separata NPV, Lonomia obliqua NPV, Lymantria dispar MNPV, Lymantria xylina NPV, Mamestra brassicae MNPV, Mamestra configurata NPV A, Mamestra configurata NPVB, Maruca vitrata NPV, Mythimna unipuncta NPV A, Mythimna unipuncta NPV B, Operophtera brumata NPV, Orgyia leucostigma NPV, Orgyia pseudotsugata MNPV, Oxyplax ochracea NPV, Peridroma saucia NPV, Perigonia lusca NPV, Spodoptera eridania NPV, Spodoptera exempta NPV, Spodoptera exigua multiple NPV A, Spodoptera exigua multiple NPV B, Spodoptera frugiperda MNPV, Spodoptera littoralis NPV, Spodoptera litura NPV, Sucra jujuba NPV, Thysanoplusia orichalcea NPV (ThorNPV), Trichoplusia ni single NPV, Urbanus proteus NPV, Wiseana signata NPV, or a variant strain of any of the preceding alpha-baculoviruses.

In some embodiments, the reference baculovirus genome is the genome of a beta-baculovirus selected from: Adoxophyes orana granulovirus (GV), Agrotis segetum GV, Choristoneura fumiferana GV, Clostera anachoreta GV, Clostera anastomosis GV A, Clostera anastomosis GV B, Cnaphalocrocis medinalis GV, Cryptophlebia leucotreta GV, Cydia pomonella GV, Diatraea saccharalis GV, Epinotia aporema GV, Erinnyis ello GV, Harrisina brillians GV, Mocis latipes GV, Mythimna unipuncta GV A, Mythimna unipuncta GV B, Phthorimaea operculella GV, Plodia interpunctella GV, Plutella xylostella GV, Spodoptera frugiperda GV, Spodoptera litura GV, Trichoplusia ni GV, Xestia c-nigrum GV, or a variant strain of any of the preceding beta-baculoviruses.

In some embodiments, the reference baculovirus genome is the genome of the delta-baculovirus Culex nigripalpus NPV or a variant strain thereof.

In some embodiments, the reference baculovirus genome is the genome of a gamma-baculovirus selected from Neodiprion lecontei NPV, Neodiprion sertifer NPV, or a variant strain thereof.

In some embodiments, the variant baculovirus genome comprises a nucleotide sequence or a portion thereof from a reference baculovirus genome chosen from AcMNPV (e.g., an AcMNPV strain E2, C6, or HR3), BmNPV, AgMNPV, OpMNPV, or ThorMNPV. In some embodiments, the variant baculovirus genome comprises a nucleotide sequence or a portion thereof from a AcMNPV (e.g., an AcMNPV strain E2, C6, or HR3) baculovirus genome. In some embodiments, the variant baculovirus genome comprises a nucleotide sequence or a portion thereof from a BmNPV baculovirus genome. In some embodiments, the variant baculovirus genome comprises a nucleotide sequence or a portion thereof from a ThorMNPV baculovirus genome.

In some embodiments, the reference baculovirus genome is a wild-type baculovirus genome described herein or a modified baculovirus genome described herein, e.g., a baculovirus genome having a deletion in one or more non-essential genes (e.g., auxiliary or per os infectivity factor gene) or regulatory regions of the one or more non-essential genes), e.g., a baculovirus genome having a deletion in a polyhedrin (polH) locus. In some embodiments, the modification to the non-essential gene or genes is an alteration that results in a reduction or elimination of a gene product, e.g., deletion, insertion, mutation (e.g., frameshift mutation), promoter modification, or insertion of a heterologous DNA adjacent to a non-essential gene.

In some embodiments, the reference baculovirus genome is a bMON14272, vAce25ko, or vAclef11KO baculovirus genome.

In some embodiments, the variant baculovirus genome comprises one or more disruptions, e.g., one or more mutations (e.g., frameshift mutations), one or more deletions, one or more insertions, or inactivation, of one or more non-essential genes (e.g., auxiliary or per os infectivity factor gene) described herein. In some embodiments, the variant baculovirus genome comprises a disruption, e.g., a mutation, a deletion, an insertion, or inactivation, of a non-essential gene.

In some embodiments, the variant baculovirus genome comprises a disruption, e.g., a mutation (e.g., frameshift mutation), a deletion, an insertion, or inactivation, of at least 1, 2, 3, 4, 5, 6, 7, 8, 9, 10, or 1-64, 1-60, 1-55, 1-50, 1-45, 1-40, 1-35, 1-30, 1-25, 1-20, 1-15, 1-10, 1-5, 1-4, 1-3, 1-2, 2-64, 2-60, 2-55, 2-50, 2-45, 2-40, 2-35, 2-30, 2-25, 2-20, 2-15, 2-10, 2-5, 2-4, 2-3, 3-64, 3-60, 3-55, 3-50, 3-45, 3-40, 3-35, 3-30, 3-25, 3-20, 3-15, 3-10, 3-5, 3-4, 4-64, 4-60, 4-55, 4-50, 4-45, 4-40, 4-35, 4-30, 4-25, 4-20, 4-15, 4-10, 4-5, 5-64, 5-60, 5-55, 5-50, 5-45, 5-40, 5-35, 5-30, 5-25, 5-20, 5-15, 5-10, 10-64, 10-60, 10-55, 10-50, 10-45, 10-40, 10-35, 10-30, 10-25, 10-20, 10-15, 15-64, 15-60, 15-55, 15-50, 15-45, 15-40, 15-35, 15-30, 15-25, 15-20, 20-64, 20-60, 20-55, 20-50, 20-45, 20-40, 20-35, 20-30, 20-25, 25-64, 25-60, 25-55, 25-50, 25-45, 25-40, 25-35, 25-30, 30-64, 30-60, 30-55, 30-50, 30-45, 30-40, 30-35, 35-64, 35-60, 35-55, 35-50, 35-45, 35-40, 40-64, 40-60, 40-55, 40-50, 40-45, 45-64, 45-60, 45-55, 45-50, 50-64, 50-60, 50-55, 55-64, 55-60, 60-64, 1, 2, 3, 4, 5, 6, 7, 8, 9, 10, 12, 14, 16, 18, 20, 25, 30, 35, 40, 45, 50, 55, 60, 62, or 64 non-essential genes. In some embodiments, the non-essential genes are selected from Ptp, Bro, Ctx, orf603, polyhedrin, egt, bv/odv-e26, ac18, pif-2, env-prot, iap-1, Sod, Fgf, v-ubi, p43, odv-e66, gp37, odv-nc42, ac69, iap-2, pnk/pnl, ac91, odv-e28, pif-4, pif-3, pif-1, pk-2, chiA, v-cath, pp34, 94K, p26, p10, p74, ac145, odv-e56, ac150, ac11, ac30, Gta, ac63, 15k, ac97, ac121, ac140, ac146, ac149, hispP, ac44, ac47, ac84, ac112/113 Nt, ac112/113Ct, ac118, ac122, ie-01, ac152, Pena, ac56, hcf-1, ac85, cg30, ac116, and ac117. In some embodiments, the non-essential gene is selected from 1, 2, 3, 4, 5, 6, 7, 8, 9, 10, 11, 12, 13, 14, 15, 16, 17, 18, 19, 20, or 21 of egt, p74 (PIF0), p26, SOD, ChiA, v-cath, p10, polyhedrin, ctx, odv-e56, PIF1, PIF2, PIF3, PIF4, PIF5, Tn7, AcORF-91, AcORF-108, AcORF-52, v-ubi, or p94. In some embodiments, the variant baculovirus genome comprises a deletion in the v-cath locus. In some embodiments, the variant baculovirus genome comprises a deletion in the polyhedrin (pohH) locus.

In some embodiments, the baculovirus expression construct or variant baculovirus genome comprises a modification, e.g., an insertion, deletion, substitution, or mutation (e.g., frame-shift mutation) in the following loci of the variant baculovirus genome: (i) v-cath and egt, (ii) v-cath, egt, and SOD1, (iii) chiA, v-cath, egt, p26, p10, p74, and SOD, (iv) chiA, v-cath, egt, p26, p10, p74, and SOD, or (v) chiA, v-cath, egt, p26, p10, p74, SOD, AcORF-91, and AcORF-108. In some embodiments, the modification comprises a deletion of a chiA gene, a v-cath gene, a p26 gene, a p10 gene, and/or a p74 gene, or a portion thereof. In some embodiments, the modification comprises an insertion of a heterologous sequence in the non-essential gene or adjacent region. In some embodiments, the modification comprises one or more mutations in the non-essential gene or adjacent region. In some embodiments, the non-essential genes are present near (e.g., downstream or upstream) of a homologous repeat region 5 (hr5).

In some embodiments, the variant baculovirus genome comprises a heterologous or foreign nucleotide sequence of interest. In some embodiments, the sequence of interest encodes a polypeptide of interest or nucleic acid of interest. In some embodiments, the polypeptide or nucleic acid of interest is a therapeutic polypeptide (e.g., therapeutic protein) or a therapeutic nucleotide sequence (e.g., a therapeutic RNAi). In some embodiments, the polypeptide of interest is a detectable marker gene, such as green fluorescent protein (GFP) or secreted embryonic alkaline phosphatase (SEAP). In some embodiments, the sequence of interest comprises one or more adeno-associated viral (AAV) gene encoding regions and/or a payload. In some embodiments, the sequence of interest is modified to remove one or more or all recognition sites of the restriction enzyme (e.g., a selected type IIS restriction enzyme) used to generate the fragments described herein (e.g., subgenomic fragments and/or subfragments). In some embodiments, the nucleotide sequence of interest is chemically synthesized. In some embodiments, the nucleotide sequence of interest is generated by a PCR-based method. In some embodiments, the nucleotide sequence of interest sequence is generated by a combination of chemical synthesis and a PCR-based method. In some embodiments, the nucleotide sequence of interest is a non-templated nucleotide sequence of interest (e.g., non-templated fragment). In some embodiments, the nucleotide sequence of interest is inserted into the variant baculovirus genome using available restriction enzyme sites. In some embodiments, the nucleotide sequence of interest is inserted into the variant baculovirus genome using Gibson Assembly™

In some embodiments, the variant baculovirus genome is at least 10 kilobases (kb), for example, at least 15 kb, at least 20 kb, at least 25 kb, at least 30 kb, at least 35 kb, at least 40 kb, at least 50 kb, at least 55 kb, at least 60 kb, at least 65 kb, at least 70 kb, at least 75 kb, at least 80 kb, at least 85 kb, at least 90 kb, at least 95 kb, at least 100 kb, at least 120 kb, at least 140 kb, at least 160 kb, at least 180 kb, at least 200 kb, at least 250 kb, at least 300 kb, at least 350 kb, or at least 400 kb in length. In some embodiments, the variant baculovirus genome is 1-500 kb, e.g., 1-450 kb, 1-400 kb, 1-350 kb, 1-300 kb, 1-250 kb, 1-200 kb, 1-150 kb, 1-100 kb, 1-50 kb, 50-500 kb, 50-450 kb, 50-400 kb, 50-350 kb, 50-300 kb, 50-250 kb, 50-200 kb, 50-150 kb, 50-100 kb, 100-500 kb, 100-450 kb, 100-400 kb, 100-350 kb, 100-300 kb, 100-250 kb, 100-200 kb, 100-150 kb, 150-500 kb, 150-450 kb, 150-400 kb, 150-350 kb, 150-300 kb, 150-250 kb, 150-200 kb, 200-500 kb, 200-450 kb, 200-400 kb, 200-350 kb, 200-300 kb, 200-250 kb, 250-500 kb, 250-450 kb, 250-400 kb, 250-350 kb, 250-300 kb, 300-500 kb, 300-450 kb, 300-400 kb, 300-350 kb, 350-500 kb, 350-450 kb, 350-400 kb, 400-500 kb, 400-450 kb, or 450-500 kb in length.

In some embodiments, the variant baculovirus genome is generated in vitro. In some embodiments, the variant baculovirus genome is generated in silico. In some embodiments, the variant baculovirus genome is designed in silico.

In some embodiments, the variant baculovirus genome described herein is present in a vector, such as a bacterial artificial chromosome (BAC). Accordingly, provided herein is a baculovirus expression construct comprising the variant baculovirus genome described herein. In some embodiments, the backbone vector (e.g., a BAC) into which the variant baculovirus genome described herein is inserted is modified such that it has one or more (e.g., at least 1, at least 2, at least 3, at least 4, at least 5, at least 6, at least 7, at least 8, at least 9, at least 10, at least 20, at least 30, at least 40, at least 50, at least 60, 1-60, 1-50, 1-40, 1-30, 1-20, 1-10, 1-5, 10-60, 10-50, 10-40, 10-30, 10-20, 20-60, 20-50, 20-40, 20-30, 30-60, 30-50, 30-40, 40-60, 40-50, or 50-60) fewer restriction enzyme sites (e.g., type IIS restriction enzyme sites) than a wild-type version of the backbone vector.

In some embodiments, other than two recognition sites of a selected restriction enzyme (e.g., selected type II restriction enzyme (e.g., type IIS restriction enzyme)) into which the variant baculovirus genome described herein is inserted, the backbone vector (e.g., BAC) is devoid of recognition sites for the selected restriction enzyme (e.g., selected type II restriction enzyme (e.g., type IIS restriction enzyme)). In some embodiments, other than two recognition sites of a selected type II restriction enzyme (e.g., type IIS restriction enzyme) into which the variant baculovirus genome described herein is inserted, the backbone vector (e.g., BAC) is devoid of recognition sites for the selected type II restriction enzyme site (e.g., type IIS restriction enzyme site). In some embodiments, the selected type II restriction enzyme (e.g., type IIS restriction enzyme) is the same selected type II restriction enzyme (e.g., type IIS restriction enzyme) used to generate the subgenomic fragments. In some embodiments, the selected type IIS restriction enzyme is BsaI. In some embodiments, the selected type IIS restriction enzyme is BsmBI. In some embodiments, the selected type II restriction enzyme (e.g., type IIS restriction enzyme) is the same selected type II restriction enzyme used to generate the subgenomic fragments.

In some embodiments, the variant baculovirus genome described herein and/or baculovirus expression construct described herein is replication competent. In some embodiments, the variant baculovirus genome described herein and/or baculovirus expression construct described herein is capable of producing functional baculovirus. In some embodiments, the variant baculovirus genome described herein and/or baculovirus expression construct described herein is capable of producing at least 50% (e.g., at least 55%, at least 60%, at least 65%, at least 70%, at least 75%, at least 80%, at least 85%, at least 90%, at least 95%, at least 100%, at least 150%, at least 200%, 50-200%, 50-150%, 50-100%, 75-200%, 75-150%, 75-100%, 100-200%, 100-150%, or 150-200%) of the baculovirus produced by a reference viral genome. In some embodiments, the variant baculovirus genome described herein and/or baculovirus expression construct described herein is capable of producing 80-120% (e.g., 85-115%, 90-110%, 95-105%, 95%, 96%, 97%, 98%, 99%, 100%, 101%, 102%, 103%, 104%, 105%) of the baculovirus produced by a reference viral genome. In some embodiments, the amount of baculovirus produced is determined by measuring viral titer (e.g., TCID50), e.g., as determined using qPCR.

### Methods of Producing a Variant Baculovirus Genome

Provided herein are methods for generating the variant baculovirus genomes described herein.

In some embodiments, the method for generating a variant baculovirus genome (e.g., a variant baculovirus genome described herein) comprises: (i) providing a plurality of fragments (e.g., a plurality of fragments as described herein), e.g., subgenomic fragments or subfragments (e.g., subgenomic fragments or subfragments described herein), wherein each fragment comprises: (a) a unique 5′ overhang and a unique 3′ overhang (e.g., unique 5′ and 3′ overhangs described herein); (b) a variant baculovirus nucleotide sequence (e.g., a variant baculovirus nucleotide sequence described herein) comprising fewer functional type II restriction enzyme sites (e.g., type IIS restriction enzyme sites) (e.g., at least 5 fewer, e.g., at least 20, 19, 18, 17, 16, 15, 14, 13, 12, 11, 10, 9, 8, 7, 6, 5, 4, 3, 2, 1, 1-20, 1-15, 1-10, 1-5, 5-20, 5-15, 5-10, 10-20, or 10-15 fewer functional type II restriction enzyme sites (e.g., type IIS restriction enzyme sites)), relative to a nucleotide sequence in a reference baculovirus genome (e.g., a nucleotide sequence in a wild-type baculovirus genome); (ii) introducing a modification (e.g., insertion, substitution, or deletion), e.g., one or more modifications, into one or more fragments comprising the variant baculovirus nucleotide sequence; and (iii) incubating the plurality of fragments under conditions suitable to form a variant baculovirus genome, thereby generating the baculovirus genome.

In some embodiments, the method for generating a variant baculovirus genome (e.g., a variant baculovirus genome described herein) comprises: (i) providing a plurality of fragments (e.g., a plurality of fragments as described herein), e.g., subgenomic fragments or subfragments (e.g., subgenomic fragments or subfragments described herein), wherein each fragment comprises: (a) a unique 5′ overhang and a unique 3′ overhang (e.g., unique 5′ and 3′ overhangs described herein); (b) a variant baculovirus nucleotide sequence (e.g., a variant baculovirus nucleotide sequence described herein) comprising fewer functional type II restriction enzyme sites (e.g., type IIS restriction enzyme sites) (e.g., at least 5 fewer, e.g., at least 20, 19, 18, 17, 16, 15, 14, 13, 12, 11, 10, 9, 8, 7, 6, 5, 4, 3, 2, 1, 1-20, 1-15, 1-10, 1-5, 5-20, 5-15, 5-10, 10-20, or 10-15 fewer functional type II restriction enzyme sites (e.g., type IIS restriction enzyme sites)), relative to a nucleotide sequence in a reference baculovirus genome (e.g., a nucleotide sequence in a wild-type baculovirus genome); wherein one or more fragments of the plurality comprise a modification (e.g., insertion, substitution, or deletion), e.g., one or more modifications, in the variant baculovirus nucleotide sequence; and (iii) incubating the plurality of fragments under conditions suitable to form a variant baculovirus genome, thereby generating the baculovirus genome.

In some embodiments, one or more fragments (e.g., subfragment or subgenomic fragment) of the plurality comprises at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 56, at least 57, at least 58, at least 59, or at least 60 fewer restriction enzyme sites (e.g., naturally occurring restriction enzyme sites), e.g., type II restriction enzyme sites (e.g., type IIS restriction enzyme sites), relative to the nucleotide sequence in a reference baculovirus genome described herein (e.g., a wild-type baculovirus genome). In some embodiments, one or more fragments of the plurality of fragments comprises a variant nucleotide sequence comprising 1-60, 2-60, 4-60, 6-60, 8-60, 10-60, 15-60, 20-60, 25-60, 30-60, 35-60, 40-60, 45-60, 50-60, 55-60, 1-50, 2-50, 4-50, 6-50, 8-50, 10-50, 15-50, 20-50, 25-50, 30-50, 35-50, 40-50, 45-50, 1-40, 2-40, 4-40, 6-40, 8-40, 10-40, 15-40, 20-40, 25-40, 30-40, 35-40, 1-30, 2-30, 4-30, 6-30, 8-30, 10-30, 15-30, 20-30, 25-30, 1-20, 2-20, 4-20, 6-20, 8-20, 10-20, 15-20, 1-10, 2-10, 4-10, 6-10, or 8-10 fewer restriction enzyme sites (e.g., naturally occurring restriction enzyme sites), e.g., type II restriction enzyme sites (e.g., type IIS restriction enzyme sites), relative to the nucleotide sequence in a reference baculovirus genome. In some embodiments, the restriction enzyme site is a recognition site of a type II restriction enzyme chosen from, but not limited to, Eco31I, BsaI Bso311, BspTNI, Esp3I, BpiI, Alw26I, BsmAI, BstMAI, BmsI, BhaI, BscAI, BscUI, BsmNI, BspST5I, Bst19I, CjeP338II, LweI, Mva1312II, PhaI, SfaNI, LguI, BspQI, PsiSI, SapI, Alw44I, ApaLI, VneI, BamHI, BcnI, BglII, Bpu1102I, BlpI, Bsp1720I, CeIII, Bsp1191, AsuII, Bpu14I, BspT104I, BstBI, Csp45I, NspV, SfuI, Bsp120I, PspOMI, Bsu15I, BanIII, Bsa29I, BseCI, BshVI, BspDI, BspXI, BsuTUI, ClaI, Cfr10I, Bse118I, BsrFI, BssAI, Cfr13I, AspS9I, BmgT120I, PspPI, Sau96I, Csp6I, AfaI, CviQI, RsaNI, Eam11041, Bst6I, Earl, EcoO109I, DraII, EcoRI, Eco47I, AvaII, Bme18I, SinI, VpaK11BI, Eco52I, BseX3I, BstZI, EagI, EclXI, Eco81I, AxyI, Bse21I, Bsu36I, Eco88I, Ama87I, AvaI, BmeT110I, BsiHKCI, BsoBlm, Eco91I, BstEII, BstPI, EcoO65I, PspEI, Eco130I, BssT1I, EcoT14I, ErhI, StyI, HinlI, AcyI, BsaHI, BssNI, BstACI, Hsp92I, AspLEI, BstHHI, CfoI, HinPIlI, HspAI, HindIII, HpaII, BsiSI, HapII, Kpn2I, AccIII, Aor13HI, BseAI, Bsp13I, BspEI, MroI, MspI, BsiSI, HapII, MvaI, AjnI, BseBI, Bst2UI, BstNI, BstOI, Psp6I, PspGI, MluI, NcoI, Bsp19I, NdeI, FauNDI, NotI, CciNI, SalII, TaqI, XbaI, XhoI, BelI, CpoI, CspI, Rsr2I, RsrII, MunI, MfeI, Psp5II, PpuMI, PspPPI, Bsp143I, BfuCI, BssMI, BstKTI, BstMBI, DpnII, Kzo9I, NdeII, Sau3Aim, HinfI, MboI, BfuCI, BssMI, BstKTI, BstMBI, DpnII, Kzo9I, NdeII, Sau3Alm, BspTI, AfIII, BfrI, Bst98I, BstAFI, MspCI, Vha4641, Pfl23II, BsiWI, PspLI, Acc65I, Asp718I, VspI, AseI, PshBI, Bsp1407I, BsrGI, BstAUI, Psp1406I, AclI, NheI, AsuNHI, BmtI, Tru1I, MseI, Tru9I, BshNI, AccB1I, BanI, BspT107I, BseDI, BsaJI, BssECI, BfmI, BstSFI, SfcI, Bpu10I, BcuI, AhII, SpeI, PagI, CciI, BspHI, ReaI, TatI, XagI, BstENI, EcoNI, PsyI, AspI, PflFI, Tth111I, TasI, Sse9I, Tsp509I, TspEI, XapI, AcsI, ApoI, Bme1390I, BmrFI, BssKI, BstSCI, MspR9I, ScrFI, StyD4I, BseXI, BbvI, BstV1I, BshTI, AgeI, AsiGI, CspAI, PinAI, XmiI, AccI, FbII, NmuCI, Tsp45I, PsuI, BstX2I, BstYI, MflI, XhoII, XmaJI, AspA2I, AvrII, BinI, SatI, Fnu4HI, Fsp4HI, ItaI, BveI, Acc36I, BfuAI, BspMI, PfoI, FspBI, BfaI, MaeI, XspI, PfeI, TfiI, SsiI, AciI, BspACI, FaqI, BsiFI, BsmFI, HpyF3I, BstDEI, DdeI, SgsI, AscI, PalAI, CseI, HgaI, Lsp1109I, BbvI, BstV1I, CsiI, MabI, SexAI, PteI, BsePlm, BssHII, FokI, BstF5I, BtsCI, KflI, Sse1825I, SanDI, SaqAI, MseI, Tru1I, or Tru9I, or an isoschizomers of any of the aforesaid restriction enzymes. In some embodiments, the restriction enzyme is selected from AcuI, AiwI, AaR1, BaeI, BbsI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, Earl, EciI, Esp3I, FauI, FokI, HgaI, HphI, HpyAV, MbolI, MlyI, MnlI, Nme-AIII, PaqCI, PleI, SapI, SfaNI, AbsI, Acc65I, AgeI, AgsI, ArsI, AscI, AsiGI, AsiSI, Asp718I, AspA2I, AsuNHI, AvrII, Alw44I, AxyI, BamHI, BelI, BcnI, BglII, Bpu1102I, BlnI, BmtI, BsaI, Bse21I, BshTI, BsmBI, Bso31I, BspOI, BspTNI, BstENI, Bsp119I, Bsp120I, Bsu15I, Bsu36I, CciNI, Cfr13I, Csp6I, Cfr9I, CspAI, Ec1136II, Eco31I, Eco53kI, Eco81I, Eco91I, Eco52I, EcoICRI, EcoNI, EcoRI, Esp3I, FseI, Hin6I, HindIII, HpaII, I-CeuI, I-PpoI, I-SceI, KpnI, Kpn2I, LmnI, LpnPI, MauBI, MspI, MluI, NcoI, NdeI, NheI, NotI, PalAI, PI-PspI, PI-SceI, PinAI, Psp124BI, RgaI, RigI, SacI, SalI, SbfI, SdaI, SfaAI, SgfI, SgsI, SmaI, SrfI, Sse83871, SstI, TaqI, TspMI, XagI, XbaI, XhoI, XmaI, XmaCI, XmaJI, MunI, HinfI, BspTI, Pfl23II, VspI, Bsp1407I, Psp1406I, Tru1I, Bpu10I, BcuI, PagI, BshTI, XmaJI, PfoI, SgsI, PteI, KflI, or an isoschizomer of any of the aforesaid restriction enzymes. In some embodiments, the restriction enzyme is selected from AbsI, Acc65I, AgeI, AgsI, ArsI, AscI, AsiGI, AsiSI, Asp718I, AspA2I, AsuNHI, AvrII, Alw44I, AxyI, BamHI, BelI, BcnI, BglII, Bpu1102I, BlnI, BmtI, BsaI, Bse21I, BshTI, BsmBI, Bso31I, BspOI, BspTNI, BstENI, Bsp119I, Bsp120I, Bsu15I, Bsu36I, CciNI, Cfr13I, Csp6I, Cfr9I, CspAI, Ec1136II, Eco31I, Eco53kI, Eco81I, Eco91I, Eco52I, EcoICRI, EcoNI, EcoRI, Esp3I, FseI, Hin6I, HindIII, HpaII, I-CeuI, I-PpoI, I-SceI, KpnI, Kpn2I, LmnI, LpnPI, MauBI, MspI, MluI, NcoI, NdeI, NheI, NotI, PalAI, PI-PspI, PI-SceI, PinAI, Psp124BI, RgaI, RigI, SacI, SalI, SbfI, SdaI, SfaAI, SgfI, SgsI, SmaI, SrfI, Sse83871, SstI, TaqI, TspMI, XagI, XbaI, XhoI, XmaI, XmaCI, XmaJI, MunI, HinfI, BspTI, Pfl23II, VspI, Bsp1407I, Psp1406I, Tru1I, Bpu10I, BcuI, PagI, BshTI, XmaJI, PfoI, SgsI, PteI, KflI, or an isoschizomer of any of the aforesaid restriction enzymes. In some embodiments, the restriction enzyme is AgeI, AvrII, BamHI, NheI, AscI, NotI, or an isoschizomer of any of the aforesaid restriction enzymes. In some embodiments, the restriction enzyme is AgeI, AvrII, BamHI, NheI, or an isoschizomer of any of the aforesaid restriction enzymes. In some embodiments, the restriction enzyme is AscI, NotI, or an isoschizomer of any of the aforesaid restriction enzymes. In some embodiments, the restriction enzyme site is a recognition site for a type IIS restriction enzyme. In some embodiments, the type IIS restriction enzyme is selected from, but not limited to, AcuI, AlwI, AaR1, BaeI, BbsI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, Earl, EciI, Esp3I, FauI, FokI, HgaI, HphI, HpyAV, MbolI, MlyI, MnlI, Nme-AIII, PaqCI, PleI, SapI, or SfaNI, or an isoschizomer of any of the aforesaid restriction enzymes. In some embodiments, each fragment of the plurality is devoid of recognition sites of a selected type IIS restriction enzyme. In some embodiments, each fragment of the plurality is devoid of recognition sites of two or more selected type IIS restriction enzymes. In some embodiments, the selected type IIS restriction enzyme is BsaI, BsmBI, or a combination of BsaI and BsmBI. Additional non-limiting enzymes (e.g., type IIS restriction enzymes) include, for example, AasI, Acc36I, AccB7I, AceIII, AcpII, Adel, AfiI, AjuI, AlfI, AloI, Alw26I, AlwNI, AlwXI, AmaCSI, ApaBI, ApyPI, AquII, AquIII, AquIV, ArsI, Asp10HII, BasI, Bbr7I, BbvI, BbvII, Bbv16II, Bce4I, Bce83I, BceCI, BcgI, Bco5I, Bco116I, BcoKI, BflI, BfuAI, BglI, Bli736I, Bme585I, BpiI, BplI, BpmI, BpuAI, BpuJI, BpuSI, BsAJI, Bsc4I, Bsc91I, Bsc107I, BscAI, Bse3DI, BseGI, BseKI, BseLI, BseMI, BseMII, BseXI, BseZI, BsgI, BsiYI, BslI, BslFI, Bso311, BsoMAI, Bsp24I, Bsp423I, BspBS31I, BspCNI, BspD6I, BsplS4I, BspKT5I, BspLU11III, BspMI, BspST5I, BspTNI, BspTS514I, BspWI, Bst6I, Bst12I, Bst19I, Bst71I, BstAPI, BstBS32I, BstFZ438I, BstGZ53I, BstHZ55I, BstlZ316I, BstMAI, BstMWI, BstOZ616I, BstPZ418I, BstTS5I, BstV1I, BstV2I, BstXI, CaiI, CjeI, CjePI, CspCI, CstMI, DraIII, DrdI, DseDI, Eam1104I, Eco31I, Eco5TI, EcoA4I, Eco57MI, EcoO44I, EcoP15I, Esp3I, Esp1396I, GsuI, HaeIV, Hin4I, Hpy178III, Hpy188III, HpyF1OVI, LweI, MmeI, MwoI, PfiBI, PfiMI, PhaI, PpiI, PsrI, RleAI, SdiI, SfiI, SmuI, Sth132I, StsI, TaqII, TspDTI, Tsp8EI, TspGWI, Tth111II, Van91I, or VpaK11AI, or an isoschizomer of any of the aforesaid restriction enzymes. In some embodiments, the restriction enzyme is capable of producing a 5′ overhang upon cleavage by the restriction enzyme. In some embodiments, cleavage by the restriction enzyme results in at least one or at least two (e.g., one or two) base pairs outside the 5′ overhang in its recognition site. In some embodiments, the restriction enzyme is capable of producing a 3′ overhang upon cleavage by the restriction enzyme. In some embodiments, the restriction enzyme is heat inactivatable. In some embodiments, the restriction enzyme recognizes a stretch of 4-8 base pairs, e.g., 4 base pairs, 5 base pairs, 6 base pairs, 7 base pairs, or 8 base pairs. In some embodiments, the restriction enzyme is not EcoRI.

In some embodiments, one or more fragments of the plurality comprise a modification (e.g., insertion, substitution, or deletion), e.g., one or more modifications, in the variant baculovirus nucleotide sequence. In some embodiments, one or more fragments of the plurality comprise a modification (e.g., insertion, substitution, or deletion), e.g., one or more modifications, to one or more gene encoding sequences or one or more regulatory sequences (e.g., enhancers, promoters, introns, 5′UTR, or 3′UTR). In some embodiments, the variant sequence of a gene of interest may be generated by introducing the desired change or changes (e.g., substitutions, insertions, and/or deletions) directly into the nucleotide sequence during chemical synthesis of the relevant portion (e.g., a subgenomic fragment or subfragment) of the variant baculovirus genome. In some embodiments, one or more fragments of the plurality comprise a modification, e.g., one or more modifications, which introduces one or more recognition sites, e.g., for one or more enzymes (e.g., restriction enzymes). In some embodiments, one or more fragments of the plurality comprise a modification, e.g., one or more modifications, which introduces one or more nucleotide sequences encoding one or more nucleotide sequences of interest (e.g., a nucleotide encoding a polypeptide, such as a therapeutic polypeptide, a nucleic acid of interest, such as an RNAi agent, or AAV gene encoding regions and/or a payload). In some embodiments, one or more fragments of the plurality comprise a modification, e.g., one or more modifications, which deletes one or more nucleotides or stretches of nucleotides. In some embodiments, one or more fragments of the plurality comprise a modification, e.g., one or more modifications, such as an insertion, deletion, or substitution, in one or more non-essential baculovirus loci, e.g., a non-essential baculovirus gene (e.g., auxiliary or per os infectivity factor gene) and/or regulatory region of the non-essential baculovirus gene. In some embodiments, a modification, e.g., one or more modifications, such as an insertion, deletion, or substitution, is made to one or more or all non-essential baculovirus loci encoding a gene selected from Ptp, Bro, Ctx, orf603, polyhedrin, Get, bv/odv-e26, ac18, pif-2, env-prot, iap-1, Sod, Fgf, v-ubi, p43, odv-e66, gp37, odv-nc42, ac69, iap-2, pnk/pnl, ac91, odv-e28, pif-4, pif-3, pif-1, pk-2, chiA, v-cath, pp34, 94K, p26, p10, p74, ac145, odv-e56, ac150, ac11, ac30, Gta, ac63, 15k, ac97, ac121, ac140, ac146, ac149, hispP, ac44, ac47, ac84, ac112/113 Nt, ac112/113Ct, ac118, ac122, ie-01, ac152, Pena, ac56, hcf-1, ac85, cg30, ac116, and ac117. In some embodiments, a modification, e.g., one or more modifications, such as an insertion, deletion, or substitution, is made to non-essential baculovirus loci encoding a gene selected from 1, 2, 3, 4, 5, 6, 7, 8, 9, 10, 11, 12, 13, 14, 15, 16, 17, 18, 19, 20, or 21 of egt, p74 (PIF0), p26, SOD, ChiA, v-cath, p10, polyhedrin, ctx, odv-e56, PIF1, PIF2, PIF3, PIF4, PIF5, Tn7, AcORF-91, AcORF-108, AcORF-52, v-ubi, or p94.

In some embodiments, one or more fragments of the plurality comprise a modification, e.g., an insertion, deletion, substitution, or mutation (e.g., frame-shift mutation) in the following loci of the variant baculovirus genome: (i) v-cath and egt, (ii) v-cath, egt, and SOD1, (iii) chiA, v-cath, egt, p26, p10, p74, and SOD, (iv) chiA, v-cath, egt, p26, p10, p74, and SOD, or (v) chiA, v-cath, egt, p26, p10, p74, SOD, AcORF-91, and AcORF-108. In some embodiments, the modification comprises a deletion of a chiA gene, a v-cath gene, a p26 gene, a p10 gene, and/or a p74 gene, or a portion thereof. In some embodiments, the modification comprises an insertion of a heterologous sequence in the non-essential gene or adjacent region. In some embodiments, the modification comprises one or more mutations in the non-essential gene or adjacent region. In some embodiments, the non-essential genes are present near (e.g., downstream or upstream) of a homologous repeat region 5 (hr5).

In some embodiments, one or more fragments of a plurality of subfragments described herein are covalently linked, e.g., using a ligase (e.g., a T4 ligase), for example using Golden Gate Assembly or Gibson Assembly™, such that one or more subgenomic regions, one or more subgenomic fragments, one or more larger subfragments, a baculovirus genome, or baculovirus expression construct is formed. In some embodiments, the covalently-linked fragments are linear (e.g., linear DNA). In some embodiments, the covalently-linked fragments are circular (e.g., circular DNA), for example, when present in a vector (e.g., a BAC). In some embodiments, the fragments of a plurality are ligated in a single step to generate one or more subgenomic fragments, a variant baculovirus genome, or baculovirus expression construct.

### Methods of Producing a Plurality of Subgenomic Fragments or Subfragments

Also provided herein is a method for producing a plurality of subgenomic fragments capable of assembly into a variant baculovirus genome comprising: (i) providing a reference, e.g., parental, baculovirus genome (e.g., a reference baculovirus genome described herein); (ii) optionally, identifying one or more sites, e.g., all recognition sites recognized by a restriction enzyme, e.g., type II restriction enzyme (e.g., type IIS restriction enzyme) in the reference baculovirus genome; (iii) modifying the one or more recognition sites such that the baculovirus genome comprises at least 10 fewer functional restriction enzyme sites (e.g., naturally occurring restriction enzyme sites), e.g., type II restriction enzyme sites (e.g., type IIS restriction enzyme sites), relative to a nucleotide sequence in a reference baculovirus genome described herein (e.g., a nucleotide sequence in a wild-type baculovirus genome), thereby generating a primary template, (iv) partitioning the primary template into the plurality of subgenomic fragments, wherein each subgenomic fragment of the plurality comprises a unique 5′ overhang and a unique 3′ overhang, and wherein the subgenomic fragments are capable of ordered assembly based on complementarity of the 5′ overhang in one subgenomic fragment with the 3′ overhang in another subgenomic fragment; thereby producing the plurality of subgenomic fragments.

In some embodiments, one or more sites, e.g., all recognition sites recognized by a restriction enzyme (e.g., type II restriction enzyme (e.g., type IIS restriction enzyme)) in the reference baculovirus genome, are identified in silico using computer software. Any program which allows for the identification of a particular stretch of nucleotide sequences within a larger nucleotide sequence is suitable for identifying the number and location of recognition sites of a restriction enzyme (e.g., selected type II restriction enzyme (e.g., type IIS restriction enzyme)).

In some embodiments, the identified recognition sites are modified such that a primary template (e.g., variant baculovirus genome) is generated which has at least 1, at least 2, at least 3, at least 4, at least 5, at least 6, at least 7, at least 8, at least 9, at least 10, at least 15, at least 20, at least 25, at least 30, at least 35, at least 40, at least 45, at least 50, at least 55, at least 56, at least 57, at least 58, at least 59, or at least 60 fewer restriction enzyme sites, e.g., type II restriction enzyme sites (e.g., type IIS restriction enzyme sites) relative to the nucleotide sequence in a reference baculovirus genome described herein (e.g., a wild-type baculovirus genome). In some embodiments, the primary template comprises a variant nucleotide sequence comprising 1-60, 2-60, 4-60, 6-60, 8-60, 10-60, 15-60, 20-60, 25-60, 30-60, 35-60, 40-60, 45-60, 50-60, 55-60, 1-50, 2-50, 4-50, 6-50, 8-50, 10-50, 15-50, 20-50, 25-50, 30-50, 35-50, 40-50, 45-50, 1-40, 2-40, 4-40, 6-40, 8-40, 10-40, 15-40, 20-40, 25-40, 30-40, 35-40, 1-30, 2-30, 4-30, 6-30, 8-30, 10-30, 15-30, 20-30, 25-30, 1-20, 2-20, 4-20, 6-20, 8-20, 10-20, 15-20, 1-10, 2-10, 4-10, 6-10, or 8-10 fewer restriction enzyme sites (e.g., naturally occurring restriction enzyme sites), e.g., type II restriction enzyme sites (e.g., type IIS restriction enzyme sites), relative to the nucleotide sequence in a reference baculovirus genome. In some embodiments, the restriction enzyme site is a recognition site of a type II restriction enzyme chosen from, but not limited to, Eco31I, BsaI Bso311, BspTNI, Esp3I, BpiI, Alw26I, BsmAI, BstMAI, BmsI, BhaI, BscAI, BscUI, BsmNI, BspST5I, Bst19I, CjeP338II, LweI, Mva1312II, PhaI, SfaNI, LguI, BspQI, PsiSI, SapI, Alw44I, ApaLI, VneI, BamHI, BcnI, BglII, Bpu1102I, BlpI, Bsp1720I, CeIII, Bsp1191, AsuII, Bpu14I, BspT104I, BstBI, Csp45I, NspV, SfuI, Bsp120I, PspOMI, Bsu15I, BanIII, Bsa29I, BseCI, BshVI, BspDI, BspXI, BsuTUI, ClaI, Cfr10I, Bse118I, BsrFI, BssAI, Cfr13I, AspS9I, BmgT120I, PspPI, Sau96I, Csp6I, AfaI, CviQI, RsaNI, Eam11041, Bst6I, Earl, EcoO109I, DraII, EcoRI, Eco47I, AvaII, Bme18I, SinI, VpaK11BI, Eco52I, BseX3I, BstZI, EagI, EclXI, Eco81I, AxyI, Bse21I, Bsu36I, Eco88I, Ama87I, AvaI, BmeT1101, BsiHKCI, BsoBlm, Eco91I, BstEII, BstPI, EcoO65I, PspEI, Eco130I, BssT1I, EcoT14I, ErhI, StyI, HinlI, AcyI, BsaHI, BssNI, BstACI, Hsp92I, AspLEI, BstHHI, CfoI, HinPIlI, HspAI, HindIII, HpaII, BsiSI, HapII, Kpn2I, AccIII, Aor13HI, BseAI, Bsp13I, BspEI, MroI, MspI, BsiSI, HapII, MvaI, AjnI, BseBI, Bst2UI, BstNI, BstOI, Psp6I, PspGI, MluI, NcoI, Bsp19I, NdeI, FauNDI, NotI, CciNI, SalI, TaqI, XbaI, XhoI, BelI, CpoI, CspI, Rsr2I, RsrII, MunI, MfeI, Psp5II, PpuMI, PspPPI, Bsp143I, BfuCI, BssMI, BstKTI, BstMBI, DpnII, Kzo9I, NdeII, Sau3Aim, HinfI, MboI, BfuCI, BssMI, BstKTI, BstMBI, DpnII, Kzo9I, NdeII, Sau3Alm, BspTI, AfIII, BfrI, Bst98I, BstAFI, MspCI, Vha4641, Pfl23II, BsiWI, PspLI, Acc65I, Asp718I, VspI, AseI, PshBI, Bsp1407I, BsrGI, BstAUI, Psp1406I, AclI, NheI, AsuNHI, BmtI, Tru1I, MseI, Tru9I, BshNI, AccB1I, BanI, BspT107I, BseDI, BsaJI, BssECI, BfmI, BstSFI, SfcI, Bpu10I, BcuI, AhII, SpeI, PagI, CciI, BspHI, ReaI, TatI, XagI, BstENI, EcoNI, PsyI, AspI, PflFI, Tth111I, TasI, Sse9I, Tsp509I, TspEI, XapI, AcsI, ApoI, Bme1390I, BmrFI, BssKI, BstSCI, MspR9I, ScrFI, StyD4I, BseXI, BbvI, BstV1I, BshTI, AgeI, AsiGI, CspAI, PinAI, XmiI, AccI, FbII, NmuCI, Tsp45I, PsuI, BstX2I, BstYI, MflI, XhoII, XmaJI, AspA2I, AvrII, BinI, SatI, Fnu4HI, Fsp4HI, ItaI, BveI, Acc36I, BfuAI, BspMI, PfoI, FspBI, BfaI, MaeI, XspI, PfeI, TfiI, SsiI, AciI, BspACI, FaqI, BsiFI, BsmFI, HpyF3I, BstDEI, DdeI, SgsI, AscI, PalAI, CseI, HgaI, Lsp1109I, BbvI, BstV1I, CsiI, MabI, SexAI, PteI, BsePlm, BssHII, FokI, BstF5I, BtsCI, KflI, Sse1825I, SanDI, SaqAI, MseI, Tru1I, or Tru9I, or an isoschizomers of any of the aforesaid restriction enzymes. In some embodiments, the restriction enzyme is selected from AcuI, AlwI, AaR1, BaeI, BbsI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, Earl, EciI, Esp3I, FauI, FokI, HgaI, HphI, HpyAV, MbolI, MlyI, MnlI, Nme-AIII, PaqCI, PleI, SapI, SfaNI, AbsI, Acc65I, AgeI, AgsI, ArsI, AscI, AsiGI, AsiSI, Asp718I, AspA2I, AsuNHI, AvrII, Alw44I, AxyI, BamHI, BelI, BcnI, BglII, Bpu1102I, BlnI, BmtI, BsaI, Bse21I, BshTI, BsmBI, Bso31I, BspOI, BspTNI, BstENI, Bsp119I, Bsp120I, Bsu15I, Bsu36I, CciNI, Cfr13I, Csp6I, Cfr9I, CspAI, Ec1136II, Eco31I, Eco53kI, Eco81I, Eco91I, Eco52I, EcoICRI, EcoNI, EcoRI, Esp3I, FseI, Hin6I, HindIII, HpaII, I-CeuI, I-PpoI, I-SceI, KpnI, Kpn2I, LmnI, LpnPI, MauBI, MspI, MluI, NcoI, NdeI, NheI, NotI, PalAI, PI-PspI, PI-SceI, PinAI, Psp124BI, RgaI, RigI, SacI, SalI, SbfI, SdaI, SfaAI, SgfI, SgsI, SmaI, SrfI, Sse83871, SstI, TaqI, TspMI, XagI, XbaI, XhoI, XmaI, XmaCI, XmaJI, MunI, HinfI, BspTI, Pfl23II, VspI, Bsp1407I, Psp1406I, Tru1I, Bpu10I, BcuI, PagI, BshTI, XmaJI, PfoI, SgsI, PteI, KflI, or an isoschizomer of any of the aforesaid restriction enzymes. In some embodiments, the restriction enzyme is selected from AbsI, Acc65I, AgeI, AgsI, ArsI, AscI, AsiGI, AsiSI, Asp718I, AspA2I, AsuNHI, AvrII, Alw44I, AxyI, BamHI, BelI, BcnI, BglII, Bpu1102I, BlnI, BmtI, BsaI, Bse21I, BshTI, BsmBI, Bso31I, BspOI, BspTNI, BstENI, Bsp119I, Bsp120I, Bsu15I, Bsu36I, CciNI, Cfr13I, Csp6I, Cfr9I, CspAI, Ec1136II, Eco31I, Eco53kI, Eco81I, Eco91I, Eco52I, EcoICRI, EcoNI, EcoRI, Esp3I, FseI, Hin6I, HindIII, HpaII, I-CeuI, I-PpoI, I-SceI, KpnI, Kpn2I, LmnI, LpnPI, MauBI, MspI, MluI, NcoI, NdeI, NheI, NotI, PalAI, PI-PspI, PI-SceI, PinAI, Psp124BI, RgaI, RigI, SacI, SalI, SbfI, SdaI, SfaAI, SgfI, SgsI, SmaI, SrfI, Sse83871, SstI, TaqI, TspMI, XagI, XbaI, XhoI, XmaI, XmaCI, XmaJI, MunI, HinfI, BspTI, Pfl23II, VspI, Bsp1407I, Psp1406I, Tru1I, Bpu10I, BcuI, PagI, BshTI, XmaJI, PfoI, SgsI, PteI, KflI, or an isoschizomer of any of the aforesaid restriction enzymes. In some embodiments, the restriction enzyme is AgeI, AvrII, BamHI, NheI, AscI, NotI, or an isoschizomer of any of the aforesaid restriction enzymes. In some embodiments, the restriction enzyme is AgeI, AvrII, BamHI, NheI, or an isoschizomer of any of the aforesaid restriction enzymes. In some embodiments, the restriction enzyme is AscI, NotI, or an isoschizomer of any of the aforesaid restriction enzymes. In some embodiments, the restriction enzyme site is a recognition site for a type IIS restriction enzyme. In some embodiments, the type IIS restriction enzyme is selected from, but not limited to, AcuI, AlwI, AaR1, BaeI, BbsI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, Earl, EciI, Esp3I, FauI, FokI, HgaI, HphI, HpyAV, MbolI, MlyI, MnlI, Nme-AIII, PaqCI, PleI, SapI, or SfaNI, or an isoschizomer of any of the aforesaid restriction enzymes. In some embodiments, the primary template is devoid of recognition sites of a selected type IIS restriction enzyme. In some embodiments, the primary template is devoid of recognition sites of two or more selected type IIS restriction enzymes. In some embodiments, the selected type IIS restriction enzyme is BsaI, BsmBI, or a combination of BsaI and BsmBI. Additional non-limiting enzymes (e.g., type IIS restriction enzymes) include, for example, AasI, Acc36I, AccB7I, AceIII, AcpII, Adel, AfiI, AjuI, AlfI, AloI, Alw26I, AlwNI, AlwXI, AmaCSI, ApaBI, ApyPI, AquII, AquIII, AquIV, ArsI, Asp10HII, BasI, Bbr7I, BbvI, BbvII, Bbv16II, Bce4I, Bce83I, BceCI, BcgI, Bco5I, Bco116I, BcoKI, BflI, BfuAI, BglI, Bli736I, Bme585I, BpiI, BplI, BpmI, BpuAI, BpuJI, BpuSI, BsAJI, Bsc4I, Bsc91I, Bsc107I, BscAI, Bse3DI, BseGI, BseKI, BseLI, BseMI, BseMII, BseXI, BseZI, BsgI, BsiYI, BslI, BslFI, Bso31I, BsoMAI, Bsp24I, Bsp4231, BspBS31I, BspCNI, BspD6I, BsplS4I, BspKT5I, BspLU11111, BspMI, BspST5I, BspTNI, BspTS5141, BspWI, Bst6I, Bst12I, Bst19I, Bst711, BstAPI, BstBS32I, BstFZ438I, BstGZ53I, BstHZ55I, BstlZ316I, BstMAI, BstMWI, BstOZ616I, BstPZ418I, BstTS5I, BstV1I, BstV2I, BstXI, CaiI, CjeI, CjePI, CspCI, CstMI, DraIII, DrdI, DseDI, Eam11041, Eco31I, Eco5TI, EcoA4I, Eco57MI, EcoO44I, EcoP15I, Esp3I, Esp13961, GsuI, HaeIV, Hin4I, Hpy178III, Hpy188III, HpyF1OVI, LweI, MmeI, MwoI, PfiBI, PfiMI, PhaI, PpiI, PsrI, RleAI, SdiI, SfiI, SmuI, Sth1321, StsI, TaqII, TspDTI, Tsp8EI, TspGWI, Tth111II, Van91I, or VpaK11AI, or an isoschizomer of any of the aforesaid restriction enzymes. In some embodiments, the restriction enzyme is capable of producing a 5′ overhang upon cleavage by the restriction enzyme. In some embodiments, cleavage by the restriction enzyme results in at least one or at least two (e.g., one or two) base pairs outside the 5′ overhang in its recognition site. In some embodiments, the restriction enzyme is capable of producing a 3′ overhang upon cleavage by the restriction enzyme. In some embodiments, the restriction enzyme is heat inactivatable. In some embodiments, the restriction enzyme recognizes a stretch of 4-8 base pairs, e.g., 4 base pairs, 5 base pairs, 6 base pairs, 7 base pairs, or 8 base pairs. In some embodiments, the restriction enzyme is not EcoRI.

In some embodiments, the primary template is partitioned into a plurality of subgenomic fragments (e.g., a plurality of subgenomic fragments described herein), wherein each subgenomic fragment of the plurality comprises a unique 5′ overhang and a unique 3′ overhang (e.g., the unique 5′ and 3′ overhangs described herein), and wherein the subgenomic fragments are capable of ordered assembly based on complementarity of the 5′ overhang in one subgenomic fragment with the 3′ overhang in another subgenomic fragment, or the 3′ overhang in one subgenomic fragment with the 5′ overhang in another subgenomic fragment, as described supra.

In some embodiments, the unique 5′ overhang and unique 3′ overhang of each subgenomic fragment of the plurality results from cleavage of the fragment by a restriction enzyme, e.g., a type II restriction enzyme (e.g., a type IIS restriction enzyme). In some embodiments, the restriction enzyme is a type II restriction enzyme chosen from, but not limited to, Eco31I, BsaI Bso311, BspTNI, Esp3I, BpiI, Alw26I, BsmAI, BstMAI, BmsI, BhaI, BscAI, BscUI, BsmNI, BspST5I, Bst19I, CjeP338II, LweI, Mva1312II, PhaI, SfaNI, LguI, BspQI, PsiSI, SapI, Alw44I, ApaLI, VneI, BamHI, BcnI, BglII, Bpu1102I, BlpI, Bsp1720I, CeIII, Bsp1191, AsuII, Bpu14I, BspT104I, BstBI, Csp45I, NspV, SfuI, Bsp120I, PspOMI, Bsu15I, BanIII, Bsa29I, BseCI, BshVI, BspDI, BspXI, BsuTUI, ClaI, Cfr10I, Bse118I, BsrFI, BssAI, Cfr13I, AspS9I, BmgT120I, PspPI, Sau96I, Csp6I, AfaI, CviQI, RsaNI, Eam11041, Bst6I, Earl, EcoO109I, DraII, EcoRI, Eco47I, AvaII, Bme18I, SinI, VpaK11BI, Eco52I, BseX3I, BstZI, EagI, EclXI, Eco81I, AxyI, Bse21I, Bsu36I, Eco88I, Ama87I, AvaI, BmeT1101, BsiHKCI, BsoBlm, Eco91I, BstEII, BstPI, EcoO65I, PspEI, Eco130I, BssT1I, EcoT14I, ErhI, StyI, Hin1I, AcyI, BsaHI, BssNI, BstACI, Hsp92I, AspLEI, BstHHI, CfoI, HinPIlI, HspAI, HindIII, HpaII, BsiSI, HapII, Kpn2I, AccIII, Aor13HI, BseAI, Bsp13I, BspEI, MroI, MspI, BsiSI, HapII, MvaI, AjnI, BseBI, Bst2UI, BstNI, BstOI, Psp6I, PspGI, MluI, NcoI, Bsp19I, NdeI, FauNDI, NotI, CciNI, SalI, TaqI, XbaI, XhoI, BelI, CpoI, CspI, Rsr2I, RsrII, MunI, MfeI, Psp5II, PpuMI, PspPPI, Bsp143I, BfuCI, BssMI, BstKTI, BstMBI, DpnII, Kzo9I, NdeII, Sau3Aim, HinfI, MboI, BfuCI, BssMI, BstKTI, BstMBI, DpnII, Kzo9I, NdeII, Sau3Alm, BspTI, AfiII, BfrI, Bst98I, BstAFI, MspCI, Vha464I, Pfl23II, BsiWI, PspLI, Acc65I, Asp718I, VspI, AseI, PshBI, Bsp1407I, BsrGI, BstAUI, Psp1406I, AclI, NheI, AsuNHI, BmtI, Tru1I, MseI, Tru9I, BshNI, AccB1I, BanI, BspT107I, BseDI, BsaJI, BssECI, BfmI, BstSFI, SfcI, Bpu10I, BcuI, AhII, SpeI, PagI, CciI, BspHI, ReaI, TatI, XagI, BstENI, EcoNI, PsyI, AspI, PflFI, Tth111I, TasI, Sse9I, Tsp509I, TspEI, XapI, AcsI, ApoI, Bme1390I, BmrFI, BssKI, BstSCI, MspR9I, ScrFI, StyD4I, BseXI, BbvI, BstV1I, BshTI, AgeI, AsiGI, CspAI, PinAI, XmiI, AccI, FbII, NmuCI, Tsp45I, PsuI, BstX2I, BstYI, MflI, XhoII, XmaJI, AspA2I, AvrII, BinI, SatI, Fnu4HI, Fsp4HI, ItaI, BveI, Acc36I, BfuAI, BspMI, PfoI, FspBI, BfaI, MaeI, XspI, PfeI, TfiI, SsiI, AciI, BspACI, FaqI, BsiFI, BsmFI, HpyF3I, BstDEI, DdeI, SgsI, AscI, PalAI, CseI, HgaI, Lsp11091, BbvI, BstV1I, CsiI, MabI, SexAI, PteI, BsePlm, BssHII, FokI, BstF5I, BtsCI, KflI, Sse18251, SanDI, SaqAI, MseI, Tru1I, or Tru9I, or an isoschizomers of any of the aforesaid restriction enzymes. In some embodiments, the restriction enzyme is selected from AcuI, AiwI, AaR1, BaeI, BbsI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, Earl, EciI, Esp3I, FauI, FokI, HgaI, HphI, HpyAV, MbolI, MlyI, MnlI, Nme-AIII, PaqCI, PleI, SapI, SfaNI, AbsI, Acc65I, AgeI, AgsI, ArsI, AscI, AsiGI, AsiSI, Asp718I, AspA2I, AsuNHI, AvrII, Alw44I, AxyI, BamHI, BelI, BcnI, BglII, Bpu1102I, BlnI, BmtI, BsaI, Bse21I, BshTI, BsmBI, Bso31I, BspOI, BspTNI, BstENI, Bsp1191, Bsp120I, Bsu15I, Bsu36I, CciNI, Cfr13I, Csp6I, Cfr9I, CspAI, Ec1136II, Eco31I, Eco53kI, Eco81I, Eco91I, Eco52I, EcoICRI, EcoNI, EcoRI, Esp3I, FseI, Hin6I, HindIII, HpaII, I-CeuI, I-PpoI, I-SceI, KpnI, Kpn2I, LmnI, LpnPI, MauBI, MspI, MluI, NcoI, NdeI, NheI, NotI, PalAI, PI-PspI, PI-SceI, PinAI, Psp124BI, RgaI, RigI, SacI, SalI, SbfI, SdaI, SfaAI, SgfI, SgsI, SmaI, SrfI, Sse8387I, SstI, TaqI, TspMI, XagI, XbaI, XhoI, XmaI, XmaCI, XmaJI, MunI, HinfI, BspTI, Pfl23II, VspI, Bsp1407I, Psp1406I, Tru1I, Bpu10I, BcuI, PagI, BshTI, XmaJI, PfoI, SgsI, PteI, KflI, or an isoschizomer of any of the aforesaid restriction enzymes. In some embodiments, the restriction enzyme is selected from AbsI, Acc65I, AgeI, AgsI, ArsI, AscI, AsiGI, AsiSI, Asp718I, AspA2I, AsuNHI, AvrII, Alw44I, AxyI, BamHI, BelI, BcnI, BglII, Bpu1102I, BlnI, BmtI, BsaI, Bse21I, BshTI, BsmBI, Bso31I, BspOI, BspTNI, BstENI, Bsp1191, Bsp120I, Bsu15I, Bsu36I, CciNI, Cfr13I, Csp6I, Cfr9I, CspAI, Ec1136II, Eco31I, Eco53kI, Eco811, Eco91I, Eco52I, EcoICRI, EcoNI, EcoRI, Esp3I, FseI, Hin6I, HindIII, HpaII, I-CeuI, I-PpoI, I-SceI, KpnI, Kpn2I, LmnI, LpnPI, MauBI, MspI, MluI, NcoI, NdeI, NheI, NotI, PalAI, PI-PspI, PI-SceI, PinAI, Psp124BI, RgaI, RigI, SacI, SalI, SbfI, SdaI, SfaAI, SgfI, SgsI, SmaI, SrfI, Sse8387I, SstI, TaqI, TspMI, XagI, XbaI, XhoI, XmaI, XmaCI, XmaJI, MunI, HinfI, BspTI, Pfl23II, VspI, Bsp1407I, Psp1406I, Tru1I, Bpu10I, BcuI, PagI, BshTI, XmaJI, PfoI, SgsI, PteI, KflI, or an isoschizomer of any of the aforesaid restriction enzymes. In some embodiments, the restriction enzyme is AgeI, AvrII, BamHI, NheI, AscI, NotI, or an isoschizomer of any of the aforesaid restriction enzymes. In some embodiments, the restriction enzyme is AgeI, AvrII, BamHI, NheI, or an isoschizomer of any of the aforesaid restriction enzymes. In some embodiments, the restriction enzyme is AscI, NotI, or an isoschizomer of any of the aforesaid restriction enzymes. In some embodiments, the unique 5′ overhang and unique 3′ overhang of each fragment of the plurality results from cleavage of the fragment by a selected type IIS restriction enzyme. In some embodiments, the type IIS restriction enzyme is selected from, but not limited to, AcuI, AlwI, AaR1, BaeI, BbsI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, Earl, EciI, Esp3I, FauI, FokI, HgaI, HphI, HpyAV, MbolI, MlyI, MnlI, Nme-AIII, PaqCI, PleI, SapI, or SfaNI, or an isoschizomer of any of the aforesaid restriction enzymes. In some embodiments, the type IIS restriction enzyme is BsaI. In some embodiments, the type IIS restriction enzyme is BsmBI. Additional non-limiting restriction enzymes (e.g., type IIS restriction enzymes) suitable for use include, for example, AasI, Acc36I, AccB7I, AceIII, AcpII, Adel, AfiI, AjuI, AlfI, AloI, Alw26I, AlwNI, AlwXI, AmaCSI, ApaBI, ApyPI, AquII, AquIII, AquIV, ArsI, Asp10HII, BasI, Bbr7I, BbvI, BbvII, Bbv16II, Bce4I, Bce83I, BceCI, BcgI, Bco5I, Bco116I, BcoKI, BflI, BfuAI, BglI, Bli736I, Bme585I, BpiI, BplI, BpmI, BpuAI, BpuJI, BpuSI, BsAJI, Bsc4I, Bsc91I, Bsc107I, BscAI, Bse3DI, BseGI, BseKI, BseLI, BseMI, BseMII, BseXI, BseZI, BsgI, BsiYI, BslI, BslFI, Bso31I, BsoMAI, Bsp24I, Bsp423I, BspBS31I, BspCNI, BspD6I, BsplS4I, BspKT5I, BspLU11111, BspMI, BspST5I, BspTNI, BspTS514I, BspWI, Bst6I, Bst12I, Bst19I, Bst71I, BstAPI, BstBS32I, BstFZ438I, BstGZ53I, BstHZ55I, BstlZ316I, BstMAI, BstMWI, BstOZ616I, BstPZ418I, BstTS5I, BstV1I, BstV2I, BstXI, CaiI, CjeI, CjePI, CspCI, CstMI, DraIII, DrdI, DseDI, Eam11041, Eco31I, Eco5TI, EcoA4I, Eco57MI, EcoO44I, EcoP15I, Esp3I, Esp13961, GsuI, HaeIV, Hin4I, Hpy178III, Hpy188III, HpyF1OVI, LweI, MmeI, MwoI, PfiBI, PfiMI, PhaI, PpiI, PsrI, RleAI, SdiI, SfiI, SmuI, Sth1321, StsI, TaqII, TspDTI, Tsp8EI, TspGWI, Tth111II, Van91I, or VpaK11AI, or an isoschizomer of any of the aforesaid restriction enzymes. In some embodiments, the restriction enzyme recognizes a discontinuous site, for example, AasI, AccB7I, AcpII, Adel, AfiI, AjuI, AlwNI, ApaBI, ArsI, Asp10HII, BaeI, BasI, Bce4I, BceCI, BcgI, BfiI, BglI, Bsc4I, Bsc107I, BseLI, BsiYI, BslI, BspWI, BstAPI, BstH255I, BstlZ316I, BstMWI, BstXI, CaiI, BraIII, DrdI, DseDI, HpyF10VI, PflBI, PflMI, SdiI, SfiI, Tsp8EI, or Van91I, or an isoschizomer of any of the aforesaid restriction enzymes. In some embodiments, the restriction enzyme is capable of producing a 5′ overhang upon cleavage by the restriction enzyme. In some embodiments, cleavage by the restriction enzyme results in at least one or at least two (e.g., one or two) base pairs outside the 5′ overhang in its recognition site. In some embodiments, the restriction enzyme is capable of producing a 3′ overhang upon cleavage by the restriction enzyme. In some embodiments, the restriction enzyme is heat inactivatable. In some embodiments, the restriction enzyme recognizes a stretch of 4-8 base pairs, e.g., 4 base pairs, 5 base pairs, 6 base pairs, 7 base pairs, or 8 base pairs. In some embodiments, the restriction enzyme is not EcoRI.

In some embodiments, the primary template is partitioned into a plurality of subgenomic fragments (e.g., a plurality of subgenomic fragments described herein), wherein each subgenomic fragment of the plurality is capable of ordered assembly with another subgenomic fragment of the plurality based on the overlap in nucleotide sequence at the 5′ end of one subgenomic fragment (e.g., a baculovirus genome sequence) with nucleotide sequence at the 3′ end of another subgenomic fragment (e.g., a baculovirus genome sequence). In some embodiments, the overlap in nucleotide sequence between two subgenomic fragments is 4-50 (e.g., 4-45, 4-40, 4-35, 4-30, 4-25, 4-20, 4-15, 4-10, 10-50, 10-45, 10-40, 10-35, 10-30, 10-25, 10-20, 10-15, 15-50, 15-45, 15-40, 15-35, 15-30, 15-25, 15-20, 20-50, 20-45, 20-40, 20-35, 20-30, 20-25, 25-50, 25-45, 25-40, 25-35, 25-30, 30-50, 30-45, 30-40, 30-35, 35-50, 35-45, 35-40, 40-50, 40-45) base pairs, e.g., contiguous base pairs. In some embodiments, following cleavage by a type II restriction enzyme disclosed herein, a 5′ exonuclease creates a single-stranded region of complementarity corresponding to the overlapping nucleotide sequence between subgenomic fragments, producing subgenomic fragments capable of annealing with each other. In some embodiments, gaps between the annealed subgenomic fragments are filled in by a DNA polymerase.

In some embodiments, each subgenomic fragment is maintained in a carrier vector (e.g., a first carrier vector). In some embodiments, the empty carrier vector lacks recognition sites of the selected restriction enzyme, e.g., type II restriction enzyme (e.g., type IIS restriction enzyme). In some embodiments, the subgenomic fragments are chemically synthesized. In some embodiments, the subgenomic fragments are chemically synthesized to include recognition sites for the selected restriction enzyme, e.g., type II restriction enzyme (e.g., type IIS restriction enzyme) at both the 5′ end and 3′ end of the subgenomic fragment. In some embodiments, the subgenomic fragments are non-templated subgenomic fragments. In some embodiments, each subgenomic fragment is blunt end ligated into the carrier vector. Any method of introducing the subgenomic fragment into a carrier vector can be used, so long as a subgenomic fragment with a unique 5′ overhang and a unique 3′ overhang is released when the carrier vector is digested with the selected restriction enzyme (e.g., type IIS restriction enzyme), or a subgenomic fragment having 5′ overhangs at its 5′ and 3′ ends is released when the carrier vector is digested with the selected type II restriction enzyme.

In some embodiments, a plurality of subfragments (e.g., a plurality of subfragments described herein) capable of assembly into a subgenomic fragment is produced by further partitioning the subgenomic fragments into smaller subfragments (e.g., subfragments as described herein).

In some embodiments, each fragment of the plurality of subfragments which forms a subgenomic fragment comprises a unique 5′ overhang and a unique 3′ overhang (e.g., the unique 5′ and 3′ overhangs described herein), wherein the subfragments are capable of ordered assembly based on complementarity of the 5′ overhang in one subfragment with the 3′ overhang in another subfragment, as described herein.

In some embodiments, the unique 5′ overhang and unique 3′ overhang of each subfragment of the plurality results from cleavage of the fragment by a restriction enzyme, e.g., a type II restriction enzyme (e.g., a type IIS restriction enzyme). In some embodiments, the restriction enzyme is a type II restriction enzyme chosen from, but not limited to, Eco31I, BsaI Bso311, BspTNI, Esp3I, BpiI, Alw26I, BsmAI, BstMAI, BmsI, BhaI, BscAI, BscUI, BsmNI, BspST5I, Bst19I, CjeP338II, LweI, Mva1312II, PhaI, SfaNI, LguI, BspQI, PsiSI, SapI, Alw44I, ApaLI, VneI, BamHI, BcnI, BglII, Bpu1102I, BlpI, Bsp1720I, CeIII, Bsp1191, AsuII, Bpu14I, BspT104I, BstBI, Csp45I, NspV, SfuI, Bsp120I, PspOMI, Bsu15I, BanIII, Bsa29I, BseCI, BshVI, BspDI, BspXI, BsuTUI, ClaI, Cfr10I, Bse118I, BsrFI, BssAI, Cfr13I, AspS9I, BmgT120I, PspPI, Sau96I, Csp6I, AfaI, CviQI, RsaNI, Eam11041, Bst6I, Earl, EcoO109I, DraII, EcoRI, Eco47I, AvaII, Bme18I, SinI, VpaK11BI, Eco52I, BseX3I, BstZI, EagI, EcIXI, Eco81I, AxyI, Bse21I, Bsu36I, Eco88I, Ama87I, AvaI, BmeT110I, BsiHKCI, BsoBlm, Eco91I, BstEII, BstPI, EcoO65I, PspEI, Eco130I, BssT1I, EcoT14I, ErhI, StyI, HinlI, AcyI, BsaHI, BssNI, BstACI, Hsp92I, AspLEI, BstHHI, CfoI, HinPIlI, HspAI, HindIII, HpaII, BsiSI, HapII, Kpn2I, AccIII, Aor13HI, BseAI, Bsp13I, BspEI, MroI, MspI, BsiSI, HapII, MvaI, AjnI, BseBI, Bst2UI, BstNI, BstOI, Psp6I, PspGI, MluI, NcoI, Bsp19I, NdeI, FauNDI, NotI, CciNI, SalI, TaqI, XbaI, XhoI, BelI, CpoI, CspI, Rsr2I, RsrII, MunI, MfeI, Psp5II, PpuMI, PspPPI, Bsp143I, BfuCI, BssMI, BstKTI, BstMBI, DpnII, Kzo9I, NdeII, Sau3Aim, HinfI, MboI, BfuCI, BssMI, BstKTI, BstMBI, DpnII, Kzo9I, NdeII, Sau3Alm, BspTI, AfIII, BfrI, Bst98I, BstAFI, MspCI, Vha464I, Pfl23II, BsiWI, PspLI, Acc65I, Asp718I, VspI, AseI, PshBI, Bsp1407I, BsrGI, BstAUI, Psp1406I, AclI, NheI, AsuNHI, BmtI, Tru1I, MseI, Tru9I, BshNI, AccB1I, BanI, BspT107I, BseDI, BsaJI, BssECI, BfmI, BstSFI, SfcI, Bpu10I, BcuI, AhII, SpeI, PagI, CciI, BspHI, ReaI, TatI, XagI, BstENI, EcoNI, PsyI, AspI, PflFI, Tth111I, TasI, Sse9I, Tsp509I, TspEI, XapI, AcsI, ApoI, Bme1390I, BmrFI, BssKI, BstSCI, MspR9I, ScrFI, StyD4I, BseXI, BbvI, BstV1I, BshTI, AgeI, AsiGI, CspAI, PinAI, XmiI, AccI, FbII, NmuCI, Tsp45I, PsuI, BstX2I, BstYI, MflI, XhoII, XmaJI, AspA2I, AvrII, BinI, SatI, Fnu4HI, Fsp4HI, ItaI, BveI, Acc36I, BfuAI, BspMI, PfoI, FspBI, BfaI, MaeI, XspI, PfeI, TfiI, SsiI, AciI, BspACI, FaqI, BsiFI, BsmFI, HpyF3I, BstDEI, DdeI, SgsI, AscI, PalAI, CseI, HgaI, Lsp1109I, BbvI, BstV1I, CsiI, MabI, SexAI, PteI, BsePlm, BssHII, FokI, BstF5I, BtsCI, KflI, Sse1825I, SanDI, SaqAI, MseI, Tru1I, or Tru9I, or an isoschizomers of any of the aforesaid restriction enzymes. In some embodiments, the restriction enzyme is selected from AcuI, AiwI, AaR1, BaeI, BbsI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, Earl, EciI, Esp3I, FauI, FokI, HgaI, HphI, HpyAV, MbolI, MlyI, MnlI, Nme-AIII, PaqCI, PleI, SapI, SfaNI, AbsI, Acc65I, AgeI, AgsI, ArsI, AscI, AsiGI, AsiSI, Asp718I, AspA2I, AsuNHI, AvrII, Alw44I, AxyI, BamHI, BelI, BcnI, BglII, Bpu1102I, BlnI, BmtI, BsaI, Bse21I, BshTI, BsmBI, Bso31I, BspOI, BspTNI, BstENI, Bsp119I, Bsp120I, Bsu15I, Bsu36I, CciNI, Cfr13I, Csp6I, Cfr9I, CspAI, Ec1136II, Eco31I, Eco53kI, Eco81I, Eco91I, Eco52I, EcoICRI, EcoNI, EcoRI, Esp3I, FseI, Hin6I, HindIII, HpaII, I-CeuI, I-PpoI, I-SceI, KpnI, Kpn2I, LmnI, LpnPI, MauBI, MspI, MluI, NcoI, NdeI, NheI, NotI, PalAI, PI-PspI, PI-SceI, PinAI, Psp124BI, RgaI, RigI, SacI, SalI, SbfI, SdaI, SfaAI, SgfI, SgsI, SmaI, SrfI, Sse83871, SstI, TaqI, TspMI, XagI, XbaI, XhoI, XmaI, XmaCI, XmaJI, MunI, HinfI, BspTI, Pfl23II, VspI, Bsp1407I, Psp1406I, Tru1I, Bpu10I, BcuI, PagI, BshTI, XmaJI, PfoI, SgsI, PteI, KflI, or an isoschizomer of any of the aforesaid restriction enzymes. In some embodiments, the restriction enzyme is selected from AbsI, Acc65I, AgeI, AgsI, ArsI, AscI, AsiGI, AsiSI, Asp718I, AspA2I, AsuNHI, AvrII, Alw44I, AxyI, BamHI, BelI, BcnI, BglII, Bpu1102I, BlnI, BmtI, BsaI, Bse21I, BshTI, BsmBI, Bso31I, BspOI, BspTNI, BstENI, Bsp119I, Bsp120I, Bsu15I, Bsu36I, CciNI, Cfr13I, Csp6I, Cfr9I, CspAI, Ec1136II, Eco31I, Eco53kI, Eco81I, Eco91I, Eco52I, EcoICRI, EcoNI, EcoRI, Esp3I, FseI, Hin6I, HindIII, HpaII, I-CeuI, I-PpoI, I-SceI, KpnI, Kpn2I, LmnI, LpnPI, MauBI, MspI, MluI, NcoI, NdeI, NheI, NotI, PalAI, PI-PspI, PI-SceI, PinAI, Psp124BI, RgaI, RigI, SacI, SalI, SbfI, SdaI, SfaAI, SgfI, SgsI, SmaI, SrfI, Sse83871, SstI, TaqI, TspMI, XagI, XbaI, XhoI, XmaI, XmaCI, XmaJI, MunI, HinfI, BspTI, Pfl23II, VspI, Bsp1407I, Psp1406I, Tru1I, Bpu10I, BcuI, PagI, BshTI, XmaJI, PfoI, SgsI, PteI, KflI, or an isoschizomer of any of the aforesaid restriction enzymes. In some embodiments, the restriction enzyme is AgeI, AvrII, BamHI, NheI, AscI, NotI, or an isoschizomer of any of the aforesaid restriction enzymes. In some embodiments, the restriction enzyme is AgeI, AvrII, BamHI, NheI, or an isoschizomer of any of the aforesaid restriction enzymes. In some embodiments, the restriction enzyme is AscI, NotI, or an isoschizomer of any of the aforesaid restriction enzymes. In some embodiments, the unique 5′ overhang and unique 3′ overhang of each subfragment of the plurality results from cleavage of the fragment by a selected type IIS restriction enzyme. In some embodiments, the type IIS restriction enzyme is selected from, but not limited to, AcuI, AlwI, AaR1, BaeI, BbsI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, Earl, EciI, Esp3I, FauI, FokI, HgaI, HphI, HpyAV, MbolI, MlyI, MnlI, Nme-AIII, PaqCI, PleI, SapI, or SfaNI, or an isoschizomer of any of the aforesaid restriction enzymes. In some embodiments, the restriction enzyme, e.g., type II restriction enzyme (e.g., type IIS restriction enzyme) used to generate subfragments differs from the restriction enzyme, e.g., type II restriction enzyme (e.g., type IIS restriction enzyme) used to generate the subgenomic fragments. In some embodiments, the restriction enzymes used to generate the subgenomic fragments and subfragments are type IIS restriction enzymes. In some embodiments, the type IIS restriction enzyme used to generate subfragments is BsaI or BsmBI, and the type IIS restriction enzyme used to generate subfragments is BsaI or BsmBI, wherein the type IIS restriction enzymes used to generate subfragments and subgenomic fragments are different. Additional non-limiting restriction enzymes (e.g., type IIS restriction enzymes) suitable for use include, for example, AasI, Acc36I, AccB7I, AceIII, AcpII, Adel, AfiI, AjuI, AlfI, AloI, Alw26I, AlwNI, AlwXI, AmaCSI, ApaBI, ApyPI, AquII, AquIII, AquIV, ArsI, Asp10HII, BasI, Bbr7I, BbvI, BbvII, Bbv16II, Bce4I, Bce83I, BceCI, BcgI, Bco5I, Bco116I, BcoKI, BflI, BfuAI, BglI, Bli736I, Bme585I, BpiI, BplI, BpmI, BpuAI, BpuJI, BpuSI, BsAJI, Bsc4I, Bsc91I, Bsc107I, BscAI, Bse3DI, BseGI, BseKI, BseLI, BseMI, BseMII, BseXI, BseZI, BsgI, BsiYI, BslI, BslFI, Bso311, BsoMAI, Bsp24I, Bsp4231, BspBS311, BspCNI, BspD6I, BsplS4I, BspKT5I, BspLU11III, BspMI, BspST5I, BspTNI, BspTS514I, BspWI, Bst6I, Bst12I, Bst19I, Bst71I, BstAPI, BstBS32I, BstFZ438I, BstGZ53I, BstHZ55I, BstlZ316I, BstMAI, BstMWI, BstOZ616I, BstPZ418I, BstTS5I, BstV1I, BstV2I, BstXI, CaiI, CjeI, CjePI, CspCI, CstMI, DraIII, DrdI, DseDI, Eam1104I, Eco31I, Eco5TI, EcoA4I, Eco57MI, EcoO44I, EcoP15I, Esp3I, Esp1396I, GsuI, HaeIV, Hin4I, Hpy178III, Hpy188III, HpyF1OVI, LweI, MmeI, MwoI, PfiBI, PfiMI, PhaI, PpiI, PsrI, RleAI, SdiI, SfiI, SmuI, Sth132I, StsI, TaqII, TspDTI, Tsp8EI, TspGWI, Tth111II, Van91I, or VpaK11AI, or an isoschizomer of any of the aforesaid restriction enzymes. In some embodiments, the restriction enzyme recognizes a discontinuous site, for example, AasI, AccB7I, AcpII, Adel, AfiI, AjuI, AlwNI, ApaBI, ArsI, Asp10HII, BaeI, BasI, Bce4I, BceCI, BcgI, BfiI, BglI, Bsc4I, Bsc107I, BseLI, BsiYI, BslI, BspWI, BstAPI, BstH255I, BstlZ316I, BstMWI, BstXI, CaiI, BraIII, DrdI, DseDI, HpyF10VI, PflBI, PflMI, SdiI, SfiI, Tsp8EI, or Van91I, or an isoschizomer of any of the aforesaid restriction enzymes. In some embodiments, the restriction enzyme is capable of producing a 5′ overhang upon cleavage by the restriction enzyme. In some embodiments, cleavage by the restriction enzyme results in at least one or at least two (e.g., one or two) base pairs outside the 5′ overhang in its recognition site. In some embodiments, the restriction enzyme is capable of producing a 3′ overhang upon cleavage by the restriction enzyme. In some embodiments, the restriction enzyme is heat inactivatable. In some embodiments, the restriction enzyme recognizes a stretch of 4-8 base pairs, e.g., 4 base pairs, 5 base pairs, 6 base pairs, 7 base pairs, or 8 base pairs. In some embodiments, the restriction enzyme is not EcoRI.

In some embodiments, each fragment of the plurality of subfragments which forms a subgenomic fragment comprises a nucleotide sequence at the 5′ end (e.g., a baculovirus genome sequence) that overlaps with (e.g., is homologous with) the nucleotide sequence at the 3′ end of another subfragment (e.g., a baculovirus genome sequence) of the plurality, wherein the nucleotide sequence in the region of overlap is unique to a pair of subfragments, wherein the subfragments are capable of ordered assembly by, e.g., Gibson Assembly™, as described herein. In some embodiments, the overlap in nucleotide sequence between two fragments is 4-50 (e.g., 4-45, 4-40, 4-35, 4-30, 4-25, 4-20, 4-15, 4-10, 10-50, 10-45, 10-40, 10-35, 10-30, 10-25, 10-20, 10-15, 15-50, 15-45, 15-40, 15-35, 15-30, 15-25, 15-20, 20-50, 20-45, 20-40, 20-35, 20-30, 20-25, 25-50, 25-45, 25-40, 25-35, 25-30, 30-50, 30-45, 30-40, 30-35, 35-50, 35-45, 35-40, 40-50, 40-45) base pairs, e.g., contiguous base pairs. In some embodiments, following cleavage by a type II restriction enzyme disclosed herein, a 5′ exonuclease creates a single-stranded region of complementarity corresponding to the overlapping nucleotide sequence between fragments, producing fragments capable of annealing with each other. In some embodiments, gaps between the annealed fragments are filled in by a DNA polymerase.

In some embodiments, each subfragment is maintained in a second carrier vector. In some embodiments, the empty second carrier vector lacks recognition sites of the selected restriction enzyme, e.g., type II restriction enzyme (e.g., type IIS restriction enzyme) used to generate the subfragments. In some embodiments, the subfragments are chemically synthesized. In some embodiments, the subfragment are chemically synthesized to include recognition sites for the selected restriction enzyme, e.g., type II restriction enzyme (e.g., type IIS restriction enzyme) at both the 5′ end and 3′ end of the subfragment. In some embodiments, the subfragments are non-templated subfragments. In some embodiments, each subfragment is blunt end ligated into the second carrier vector. Any method of introducing the subfragment into a second carrier vector can be used, so long as a subfragment with unique 5′ and 3′ overhangs is released when the second carrier vector is digested with the selected restriction enzyme, e.g., type II restriction enzyme (e.g., type IIS restriction enzyme).

In some embodiments, the second carrier vector confers resistance to an antibiotic which is not conferred by the first carrier vector for subgenomic fragments. In some embodiments, the first carrier vector for subgenomic fragments confers resistance to kanamycin, and the second carrier vector for subfragments confers resistance to ampicillin. In some embodiments, the first carrier vector for subgenomic fragments confers resistance to ampicillin, and the second carrier vector for subfragments confers resistance to kanamycin.

### Cells

Also provided herein are cells comprising the one or more subfragments, one or more subgenomic fragments, one or more subgenomic regions, a variant baculovirus genome, a baculovirus expression construct, and/or a BAC described herein, or variants thereof. In some embodiments, the cell is an insect cell. In some embodiments, the cell is a mammalian cell. In some embodiments, the cell is a bacterial cell.

In some embodiments, the insect cell comprising the one or more subfragments, one or more subgenomic fragments, one or more subgenomic regions, a variant baculovirus genome, a baculovirus expression construct, and/or a BAC described herein, or variants thereof, is a Spodopterafrugiperda cell, such as an Sf9 cell or Sf21 cell. In some embodiments, the insect cell is a Trichoplusia ni cell.

In some embodiments, the mammalian cell comprising the one or more subfragments, one or more subgenomic fragments, one or more subgenomic regions, a variant baculovirus genome, a baculovirus expression construct, and/or a BAC described herein, or variants thereof, is selected from A549, WEH1, 3T3, 10T1/2, BHK, KB, MDCK, COS 1, COS 7, C127, CHO, BSC 1, BSC 40, BMT 10, VERO, W138, HeLa, HEK293, HEK293T (293T), Saos, C2C12, L cells, HT1080, HepG2, 3T3 cells, or variant strains thereof. In some embodiments, the mammalian cell comprising the one or more subfragments, one or more subgenomic fragments, one or more subgenomic regions, a variant baculovirus genome, a baculovirus expression construct, and/or a BAC described herein, or variants thereof, is a primary fibroblast, hepatocyte, and myoblast cells derived from mammals.

In some embodiments, the bacterial cell comprising the one or more subfragments, one or more subgenomic fragments, one or more subgenomic regions, a variant baculovirus genome, a baculovirus expression construct, and/or a BAC described herein, or variants thereof, is selected from a bacterial species within the genera Escherichia, Bacillus, Pseudomonas, or Salmonella. In some embodiments, the bacterial species is E. coli.

### Compositions

Provided herein are compositions comprising the one or more subfragments, plurality of subfragments, one or more subgenomic fragments, plurality of subgenomic fragments, variant baculovirus genome, a baculovirus expression construct, and/or a bacterial artificial chromosome described herein, and a carrier. In some embodiments, the composition comprises a suitable storage buffer, e.g., phosphate-based buffer, such as phosphate-buffered saline (PBS), or a Tris-based buffer, such as TE buffer (Tris-EDTA buffer).

In some embodiments, the compositions comprise one or more baculoviruses comprising one or more subfragments, plurality of subfragments, one or more subgenomic fragments, plurality of subgenomic fragments, variant baculovirus genome, a baculovirus expression construct, and/or a bacterial artificial chromosome described herein, and a carrier. In some embodiments, the baculovirus is stored in insect cell culture medium, such as Sf-900™ II SFM.

### Kits

Provided herein are kits comprising one or more subfragments, plurality of subfragments, one or more subgenomic fragments, plurality of subgenomic fragments, variant baculovirus genome, a baculovirus expression construct, and/or a bacterial artificial chromosome described herein, and instructions for use. In some embodiments, the kit comprises a plurality of carrier vectors comprising the plurality of subgenomic fragments described herein. In some embodiments, the kit further comprises a plurality of second carrier vectors comprising the plurality of subfragments described herein. In some embodiments, the kit comprises one or more (e.g., 1, 2, 3, 4, 5, 1-5, 1-4, 1-3, 1-2, 2-5, 2-4, 2-3, 3-5, or 4-5) restriction enzymes, e.g., type II restriction enzymes, e.g., type IIS restriction enzymes.

In some embodiments, the kit comprises an empty first carrier vector (e.g., an empty first carrier vector into which the covalently-linked subfragments are inserted, or for the maintenance of subgenomic fragments). In some embodiments, the kit comprises an empty second carrier vector (e.g., an empty second carrier vector into which subfragments can be inserted, e.g., for maintenance of the subfragments). In some embodiments, the kit comprises an empty destination vector (e.g., an empty destination vector, such as a BAC, into which the covalently-linked subgenomic fragments are inserted).

The instructions for use may, for example, provide guidance for modifying a baculovirus genome based on the methods described herein.

Kits provided herein may also comprise a baculovirus comprising one or more subfragments, plurality of subfragments, one or more subgenomic fragments, plurality of subgenomic fragments, variant baculovirus genome, a baculovirus expression construct, and/or a BAC described herein, and instructions for use.

### Adeno-Associated Virus

The baculovirus expression construct (e.g., baculovirus expression construct comprising a variant baculovirus genome) or combination of baculovirus expression constructs described herein may be used, for example, to produce adeno-associated virus (AAV) particles.

Adeno-associated viruses (AAV) are small non-enveloped icosahedral capsid viruses of the Parvoviridae family characterized by a single stranded DNA viral genome. Parvoviridae family viruses consist of two subfamilies: Parvovirinae, which infect vertebrates, and Densovirinae, which infect invertebrates. The Parvoviridae family includes the Dependovirus genus which includes AAV, capable of replication in vertebrate hosts including, but not limited to, human, primate, bovine, canine, equine, and ovine species.

The parvoviruses and other members of the Parvoviridae family are generally described in Kenneth I. Berns, “Parvoviridae: The Viruses and Their Replication,” Chapter 69 in Fields Virology (3d Ed. 1996), the contents of which are incorporated by reference in their entirety.

AAV have proven to be useful as a biological tool due to their relatively simple structure, their ability to infect a wide range of cells (including quiescent and dividing cells) without integration into the host genome and without replicating, and their relatively benign immunogenic profile. The genome of the virus may be manipulated to contain a minimum of components for the assembly of a functional recombinant virus, or viral particle, which is loaded with or engineered to target a particular tissue and express or deliver a desired payload.

In some embodiments, one or more of the subgenomic fragments or subfragments described herein, variant baculovirus genome described herein, and/or baculovirus expression construct described herein, comprise a nucleotide sequence encoding one or more adeno-associated virus (AAV) genes.

In some embodiments, the AAV genes needed to produce an AAV particle are provided in one or more of the subgenomic fragments or subfragments described herein, variant baculovirus genome described herein, and/or baculovirus expression construct described herein. For example, in some embodiments, the nucleic acid sequences encoding one or more Rep proteins may be present in one or more baculovirus expression constructs described herein, and the nucleic acid sequences encoding the VP capsid proteins and payload may be present in one or more separate baculovirus expression constructs described herein.

In some embodiments, one or more of the subgenomic fragments or subfragments described herein, variant baculovirus genome described herein, and/or baculovirus expression construct described herein encodes an AAV Rep protein. In some embodiments, one or more of the subgenomic fragments or subfragments described herein, variant baculovirus genome described herein, and/or baculovirus expression construct described herein encodes Rep40, Rep52, Rep68, Rep78, or a combination thereof. In some embodiments, one or more of the subgenomic fragments or subfragments described herein, variant baculovirus genome described herein, and/or baculovirus expression construct described herein encodes a Rep52 protein and/or a Rep78 protein.

In some embodiments, one or more of the subgenomic fragments or subfragments described herein, variant baculovirus genome described herein, and/or baculovirus expression construct described herein encodes an AAV capsid protein, e.g., a VP1 protein, a VP2 protein, a VP3 protein, or a combination thereof.

In some embodiments, one or more of the subgenomic fragments or subfragments described herein, variant baculovirus genome described herein, and/or baculovirus expression construct described herein encodes an AAV1 capsid protein, an AAV2 capsid protein, an AAV3 capsid protein, an AAV4 capsid protein, an AAV5 capsid protein, an AAV6 capsid protein, an AAV8 capsid protein, an AAV9 capsid protein, an AAVrh10 capsid protein, or a variant thereof. In some embodiments, one or more of the subgenomic fragments or subfragments described herein, variant baculovirus genome described herein, and/or baculovirus expression construct described herein encodes an AAV5 capsid protein or variant thereof, or an AAV9 capsid protein or variant thereof.

In some embodiments, one or more of the subgenomic fragments or subfragments described herein, variant baculovirus genome described herein, and/or baculovirus expression construct described herein encodes a payload. In some embodiments, the encoded payload is selected from a therapeutic protein or functional variant thereof; an antibody or antibody fragment; an enzyme; a component of a gene editing system; an RNAi agent (e.g., a dsRNA, siRNA, shRNA, pre-miRNA, pri-miRNA, miRNA, stRNA, lncRNA, piRNA, or snoRNA); or a combination thereof.

Accordingly, also provided herein is a baculovirus expression construct or variant baculovirus genome comprising an AAV expression construct and/or an AAV payload construct described herein. The AAV expression constructs and/or AAV payload constructs may form part of an AAV viral production system. In some embodiments, the AAV viral production system comprises an AAV viral production cell, such as an insect cell (e.g., Sf9, Sf21).

In some embodiments, baculovirus produced using the baculovirus expression construct or combination of baculovirus expression constructs described herein produce AAV particles capable of infecting a cell (e.g., insect cells such as Sf9).

Also provided herein is a method of producing recombinant AAV (rAAV) particles in an AAV viral production cell, the method comprising: (i) providing an AAV viral production system comprising a baculovirus expression construct or variant baculovirus genome comprising an AAV expression construct and an AAV payload construct described herein, wherein the AAV expression construct comprises one or more VP-coding regions which comprise one or more nucleotide sequences encoding VP1, VP2 and VP3 capsid proteins; (ii) transfecting the AAV viral production system into an AAV viral production cell; (iii) exposing the AAV viral production cell to conditions which allow the AAV viral production cell to process the AAV expression construct and the AAV payload construct into rAAV particles; and, optionally, (iv) collecting the rAAV particles from the AAV viral production cell, e.g., an insect cell such as a Sf9 cell or a Sf21cell. Also provided are rAAV particles produced by the method, as well as pharmaceutical compositions comprising the rAAV particles and a pharmaceutically acceptable carrier or pharmaceutically acceptable excipient.

In some embodiments, the baculovirus expression construct or combination of baculovirus expression constructs comprising, e.g., an AAV expression construct and AAV payload construct, described herein produce AAV particles with titers which are at least 80% (e.g., at least 85%, at least 90%, at least 95%, at least 100%, at least 105%, at least 110%, 80-120%, 80-110%, 80-100%, 80-90%, 90-120%, 90-110%, 90-100%, 100-120%, 100-110%, or 110-120%) that of AAV particles produced from a reference baculovirus construct or combination of reference baculovirus constructs (e.g., reference baculovirus construct or combination of reference baculovirus constructs which comprise AAV gene encoding regions in equivalent positions (e.g., in the same or similar locus of the baculovirus genome) relative to the combination of baculovirus expression constructs described herein). In some embodiments, the AAV titer is determined using qPCR, for example, as described in the Examples.

Further embodiments related to AAV particles produced using the baculovirus expression construct or combination of baculovirus expression constructs described herein are provided in the sections that follow.

### AAV Serotypes and Capsids

In some embodiments, the AAV particles produced using the baculovirus expression construct or combination of baculovirus expression constructs described herein comprises a capsid polypeptide or variant thereof from any natural or recombinant AAV serotype. AAV serotypes may differ in characteristics such as, but not limited to, packaging, tropism, transduction and immunogenic profiles. While not wishing to be bound by theory, it is believed that the AAV capsid protein can modulate, e.g., direct, AAV particle tropism to a particular tissue.

In some embodiments, the AAV capsid polypeptide allows for blood brain barrier penetration following intravenous administration. In some embodiments, the AAV capsid polypeptide allows for blood brain barrier penetration following focused ultrasound (FUS), e.g., coupled with the intravenous administration of microbubbles (FUS-MB), or MRI-guided FUS coupled with intravenous administration. In some embodiments the AAV capsid polypeptide allows for increased distribution to a brain region, spinal cord region, or muscle region.

In some embodiments, the initiation codon for translation of the AAV VP1 capsid protein is CTG, TTG, or GTG as described in U.S. Pat. No. 8,163,543, the contents of which are herein incorporated by reference in its entirety.

The present disclosure refers to structural capsid proteins (including VP1, VP2 and VP3) which are encoded by capsid (Cap) genes. These capsid proteins form an outer protein structural shell (e.g. capsid) of a viral vector such as AAV. VP capsid proteins synthesized from Cap polynucleotides generally include a methionine as the first amino acid in the peptide sequence (Met1), which is associated with the start codon (AUG or ATG) in the corresponding Cap nucleotide sequence. However, it is common for a first-methionine (Met1) residue or generally any first amino acid (AA1) to be cleaved off after or during polypeptide synthesis by protein processing enzymes such as Met-aminopeptidases. This “Met/AA-clipping” process often correlates with a corresponding acetylation of the second amino acid in the polypeptide sequence (e.g., alanine, valine, serine, threonine, etc.). Met-clipping commonly occurs with VP1 and VP3 capsid proteins but can also occur with VP2 capsid proteins.

Where the Met/AA-clipping is incomplete, a mixture of one or more (one, two or three) VP capsid proteins comprising the viral capsid may be produced, some of which may include a Met1/AA1 amino acid (Met+/AA+) and some of which may lack a Met1/AA1 amino acid as a result of Met/AA-clipping (Met−/AA−). For further discussion regarding Met/AA-clipping in capsid proteins, see Jin, et al. Direct Liquid Chromatography/Mass Spectrometry Analysis for Complete Characterization of Recombinant Adeno-Associated Virus Capsid Proteins. Hum Gene Ther Methods. 2017 Oct. 28(5):255-267; Hwang, et al. N-Terminal Acetylation of Cellular Proteins Creates Specific Degradation Signals. Science. 2010 Feb. 19. 327(5968): 973-977; the contents of which are each incorporated herein by reference in its entirety.

According to the present disclosure, references to capsid proteins is not limited to either clipped (Met−/AA−) or unclipped (Met+/AA+) and may, in context, refer to independent capsid proteins, viral capsids comprised of a mixture of capsid proteins, and/or polynucleotide sequences (or fragments thereof) which encode, describe, produce or result in capsid proteins of the present disclosure. A direct reference to a capsid protein or capsid polypeptide (such as VP1, VP2 or VP2) may also comprise VP capsid proteins which include a Met1/AA1 amino acid (Met+/AA+) as well as corresponding VP capsid proteins which lack the Met1/AA1 amino acid as a result of Met/AA-clipping (Met−/AA−).

Further according to the present disclosure, a reference to a specific SEQ ID NO (whether a protein or nucleic acid) which comprises or encodes, respectively, one or more capsid proteins which include a Met1/AA1 amino acid (Met+/AA+) should be understood to teach the VP capsid proteins which lack the Met1/AA1 amino acid as upon review of the sequence, it is readily apparent any sequence which merely lacks the first listed amino acid (whether or not Met1/AA1).

As a non-limiting example, reference to a VP1 polypeptide sequence which is 736 amino acids in length and which includes a “Met1” amino acid (Met+) encoded by the AUG/ATG start codon may also be understood to teach a VP1 polypeptide sequence which is 735 amino acids in length and which does not include the “Met1” amino acid (Met−) of the 736 amino acid Met+ sequence. As a second non-limiting example, reference to a VP1 polypeptide sequence which is 736 amino acids in length and which includes an “AA1” amino acid (AA1+) encoded by any NNN initiator codon may also be understood to teach a VP1 polypeptide sequence which is 735 amino acids in length and which does not include the “AA1” amino acid (AA1−) of the 736 amino acid AA1+ sequence.

References to viral capsids formed from VP capsid proteins (such as reference to specific AAV capsid serotypes), can incorporate VP capsid proteins which include a Met1/AA1 amino acid (Met+/AA1+), corresponding VP capsid proteins which lack the Met1/AA1 amino acid as a result of Met/AA1-clipping (Met−/AA1−), and combinations thereof (Met+/AA1+ and Met−/AA1−).

As a non-limiting example, an AAV capsid serotype can include VP1 (Met+/AA1+), VP1 (Met−/AA1−), or a combination of VP1 (Met+/AA1+) and VP1 (Met−/AA1−). An AAV capsid serotype can also include VP3 (Met+/AA1+), VP3 (Met−/AA1−), or a combination of VP3 (Met+/AA1+) and VP3 (Met−/AA1−); and can also include similar optional combinations of VP2 (Met+/AA1) and VP2 (Met−/AA1−).

### Viral Genome of the AAV Particle

The wild-type AAV viral genome is a linear, single-stranded DNA (ssDNA) molecule approximately 5,000 nucleotides (nt) in length. Inverted terminal repeats (ITRs) traditionally cap the viral genome at both the 5′ and the 3′ end, providing origins of replication for the viral genome. While not wishing to be bound by theory, an AAV viral genome typically comprises two ITR sequences. These ITRs have a characteristic T-shaped hairpin structure defined by a self-complementary region (145 nt in wild-type AAV) at the 5′ and 3′ ends of the ssDNA which form an energetically stable double stranded region. The double stranded hairpin structures comprise multiple functions comprising, but not limited to, acting as an origin for DNA replication by functioning as primers for the endogenous DNA polymerase complex of the host viral replication cell.

The wild-type AAV viral genome further comprises nucleotide sequences for two open reading frames, one for the four non-structural Rep proteins (Rep78, Rep68, Rep52, Rep40, encoded by Rep genes) and one for the three capsid, or structural, proteins (VP1, VP2, VP3, encoded by capsid genes or Cap genes). The Rep proteins are important for replication and packaging, while the capsid proteins are assembled to create the protein shell of the AAV, or AAV capsid. Alternative splicing and alternate initiation codons and promoters result in the generation of four different Rep proteins from a single open reading frame and the generation of three capsid proteins from a single open reading frame. Though it varies by AAV serotype, as a non-limiting example, for AAV9/hu.14 (SEQ ID NO: 123 of U.S. Pat. No. 7,906,111, the content of which is incorporated herein by reference in its entirety as related to AAV9/hu.14, insofar as it does not conflict with the present disclosure) VP1 refers to amino acids 1-736, VP2 refers to amino acids 138-736, and VP3 refers to amino acids 203-736. In other words, VP1 is the full-length capsid sequence, while VP2 and VP3 are shorter components of the whole. As a result, changes in the sequence in the VP3 region, are also changes to VP1 and VP2, however, the percent difference as compared to the parent sequence will be greatest for VP3 since it is the shortest sequence of the three. Though described here in relation to the amino acid sequence, the nucleic acid sequence encoding these proteins can be similarly described. Together, the three capsid proteins assemble to create the AAV capsid protein. While not wishing to be bound by theory, the AAV capsid protein typically comprises a molar ratio of 1:1:10 of VP1:VP2:VP3. As used herein, an “AAV serotype” is defined primarily by the AAV capsid. In some instances, the ITRs are also specifically described by the AAV serotype (e.g., AAV2/9).

In some embodiments, the wild-type AAV viral genome can be modified to replace the rep/cap sequences with a nucleic acid sequence comprising a payload region with at least one ITR region. Typically, in recombinant AAV viral genomes there are two ITR regions. In some embodiments, the rep/cap sequences are provided in trans during production to generate AAV particles.

In addition to the encoded heterologous payload, in some embodiments, AAV vectors may comprise the viral genome, in whole or in part, of any naturally occurring and/or recombinant AAV serotype nucleotide sequence or variant. AAV variants may have sequences of significant homology at the nucleic acid (genome or capsid) and amino acid levels (capsids), to produce constructs which are generally physical and functional equivalents, replicate by similar mechanisms, and assemble by similar mechanisms. See Chiorini et al., J. Vir. 71: 6823-33(1997); Srivastava et al., J. Vir. 45:555-64 (1983); Chiorini et al., J. Vir. 73:1309-1319 (1999); Rutledge et al., J. Vir. 72:309-319 (1998); and Wu et al., J. Vir. 74: 8635-47 (2000), the contents of each of which are incorporated herein by reference in their entireties as related to AAV variants and equivalents.

In some embodiments, AAV particles, AAV genomes, and/or payloads of the present disclosure, and the methods of their use, may be as described in WO2017189963, the content of which is incorporated herein by reference in its entirety as related to AAV particles, viral genomes and/or payloads.

AAV particles of the present disclosure may be formulated in any of the gene therapy formulations of the disclosure comprising any variations of such formulations apparent to those skilled in the art. The reference to “AAV particles”, “AAV particle formulations” and “formulated AAV particles” in the present application refers to the AAV particles which may be formulated and those which are formulated without limiting either.

In some embodiments, the AAV particles are recombinant AAV (rAAV) viral particles which are replication defective, lacking sequences encoding functional Rep and Cap proteins within their viral genome. These defective AAV particles may lack most or all parental coding sequences and essentially carry only one or two AAV ITR sequences and the nucleic acid of interest (i.e., payload) for delivery to a cell, a tissue, an organ or an organism.

In some embodiments, the viral genome of the AAV particles comprises at least one control element which provides for the replication, transcription and translation of a coding sequence encoded therein. Not all of the control elements need always be present as long as the coding sequence is capable of being replicated, transcribed and/or translated in an appropriate host cell. Non-limiting examples of expression control elements comprise sequences for transcription initiation and/or termination, promoter and/or enhancer sequences, efficient RNA processing signals such as splicing and polyadenylation signals, sequences that stabilize cytoplasmic mRNA, sequences that enhance translation efficacy (e.g., Kozak consensus sequence), sequences that enhance protein stability, and/or sequences that enhance protein processing and/or secretion.

In some embodiments, AAV particles for use in therapeutics and/or diagnostics comprise a virus that has been distilled or reduced to the minimum components necessary for transduction of a nucleic acid payload or cargo of interest. In this manner, AAV particles are engineered as vehicles for specific delivery while lacking the deleterious replication and/or integration features found in wild-type viruses.

In some embodiments, AAV particles may be produced recombinantly and may be based on adeno-associated virus (AAV) parent or reference sequences.

In addition to single stranded AAV viral genomes (e.g., ssAAVs), the present disclosure also provides for self-complementary AAV (scAAVs) viral genomes. scAAV viral genomes contain DNA strands which anneal together to form double stranded DNA. By skipping second strand synthesis, scAAVs allow for rapid expression in the cell.

In some embodiments, the AAV viral genome of the present disclosure is a scAAV. In some embodiments, the AAV viral genome of the present disclosure is a ssAAV.

Methods for producing and/or modifying AAV particles are disclosed in the art, such as pseudotyped AAV particles (PCT Patent Publication Nos. WO200028004; WO200123001; WO2004112727; WO 2005005610 and WO 2005072364, the contents of each of which are incorporated herein by reference in their entireties as related to producing and/or modifying AAV particles, insofar as they do not conflict with the present disclosure).

In some embodiments, AAV particles are modified to enhance the efficiency of delivery. Such modified AAV particles can be packaged efficiently and be used to successfully infect the target cells at high frequency and with minimal toxicity. In some embodiments, the capsids of the AAV particles are engineered according to the methods described in US Publication Number US 20130195801, the content of which is incorporated herein by reference in its entirety as related to modifying AAV particles to enhance the efficiency of delivery, insofar as it does not conflict with the present disclosure.

In some embodiments, the AAV particles comprise a payload construct and/or region encoding a polypeptide or protein of the present disclosure, and may be introduced into mammalian cells. In some embodiments, the AAV particles comprise a payload construct and/or region encoding a polypeptide or protein of the present disclosure, and may be introduced into insect cells.

In some embodiments, an AAV particle is used to deliver a viral genome to a tissue. In some embodiments, an AAV particle comprising an AAV capsid polypeptide is used to deliver a viral genome to a tissue or cell. In some embodiments, the AAV particle is a recombinant AAV particle. In some embodiments, the AAV particle is an isolated AAV particle.

In some embodiments, the viral genome encodes a payload, such as, but not limited to, a polypeptide (e.g., a therapeutic polypeptide), an antibody, an enzyme, an RNAi agent and/or components of a gene editing system. In one embodiment, the AAV particles described herein are used to deliver a payload to cells of the CNS. In another embodiment, the AAV particles described herein are used to deliver a payload to cells of the DRG. In some embodiments, the AAV particles described herein are used to deliver a payload to cells of a muscle, e.g., a heart muscle.

In some embodiments, a viral genome of an AAV particle comprising an AAV capsid polypeptide comprises a nucleic acid comprising a transgene encoding a payload. In some embodiments, the viral genome comprises an inverted terminal repeat (ITR) sequence. In some embodiments, the viral genome comprises two ITR sequences, e.g., one at the 5′ end of the viral genome (e.g., 5′ relative to the encoded payload) and one at the 3′ end of the viral genome (e.g., 3′ relative to the encoded payload). In some embodiments, a viral genome of the AAV particles described herein may comprise a regulatory element (e.g., promoter), untranslated regions (UTR), a miR binding site a polyadenylation sequence (polyA), a filler or stuffer sequence, an intron, and/or a linker sequence, e.g., for enhancing transgene expression.

In some embodiments, the viral genome components are selected and/or engineered for expression of a payload in a target tissue.

### Viral Genome Component: Inverted Terminal Repeats (ITRs)

In some embodiments, the AAV particle comprising an AAV capsid polypeptide comprises a viral genome comprising an ITR and a transgene encoding a payload. In some embodiment, the viral genome has two ITRs. In some embodiments, the two ITRs flank the nucleotide sequence encoding the payload at the 5′ and 3′ ends. In some embodiments, the ITRs function as origins of replication comprising recognition sites for replication. In some embodiments, the ITRs comprise sequence regions which can be complementary and symmetrically arranged. In some embodiments, the ITRs incorporated into viral genomes as described herein may be comprised of naturally occurring polynucleotide sequences or recombinantly derived polynucleotide sequences.

In some embodiments, the ITR may be of the same serotype as the capsid polypeptide selected from any of the known serotypes (e.g., AAV2, AAV5, AAV9, or a variant thereof). In some embodiments, the ITR is a different serotype than the capsid. In one embodiment, the viral genome comprises two ITR sequence regions, wherein the ITRs are of the same serotype as one another. In another embodiment, the viral genome comprises two ITR sequence regions, wherein the ITRs are of different serotypes. Non-limiting examples include zero, one or both of the ITRs having the same serotype as the capsid. In one embodiment both ITRs of the viral genome of the AAV particle are AAV2 ITRs.

Independently, each ITR may be about 100 to about 150 nucleotides in length. An ITR may be about 100-105 nucleotides in length, 106-110 nucleotides in length, 111-115 nucleotides in length, 116-120 nucleotides in length, 121-125 nucleotides in length, 126-130 nucleotides in length, 131-135 nucleotides in length, 136-140 nucleotides in length, 141-145 nucleotides in length or 146-150 nucleotides in length. In one embodiment, the ITRs are 140-142 nucleotides in length. Non-limiting examples of ITR length are 102, 105, 130, 140, 141, 142, 145 nucleotides in length

### Viral Genome Component: Promoters

In one embodiment, the payload region of the viral genome comprises at least one element to enhance the payload target specificity and expression (See e.g., Powell et al. Viral Expression Cassette Elements to Enhance Transgene Target Specificity and Expression in Gene Therapy, 2015; the contents of which are herein incorporated by reference in their entirety). Non-limiting examples of elements to enhance payload target specificity and expression include promoters, endogenous miRNAs, post-transcriptional regulatory elements (PREs), polyadenylation (PolyA) signal sequences and upstream enhancers (USEs), CMV enhancers and introns.

In some embodiments, an AAV particle comprising an AAV capsid protein comprises a viral genome comprising a nucleic acid comprising a transgene encoding a payload, wherein the transgene is operably linked to a promoter. In some embodiments, the promoter is a species specific promoter, an inducible promoter, tissue-specific, or cell cycle-specific (Parr et al., Nat. Med. 3:1145-9 (1997); the contents of which are herein incorporated by reference in their entirety).

In some embodiments the promoter may be naturally occurring or non-naturally occurring. Non-limiting examples of promoters include those from viruses, plants, mammals, or humans. In some embodiments, the promoters may be those from human cells or systems. In some embodiments, the promoter may be truncated or mutated, e.g., a promoter variant.

In some embodiments, the promoter is a ubiquitous promoter, e.g., capable of expression in multiple tissues. In some embodiments the promoter is an human elongation factor 1α-subunit (EF1α) promoter, the cytomegalovirus (CMV) immediate-early enhancer and/or promoter, the chicken β-actin (CBA) promoter and its derivative CAG, β glucuronidase (GUSB) promoter, or ubiquitin C (UBC) promoter. In some embodiments, the promoter is a cell or tissue specific promoter, e.g., capable of expression in tissues or cells of the central or peripheral nervous systems, regions within (e.g., frontal cortex), and/or sub-sets of cells therein (e.g., excitatory neurons). In some embodiments, the promoter is a cell-type specific promoter capable of expression a payload in excitatory neurons (e.g., glutamatergic), inhibitory neurons (e.g., GABA-ergic), neurons of the sympathetic or parasympathetic nervous system, sensory neurons, neurons of the dorsal root ganglia, motor neurons, or supportive cells of the nervous systems such as microglia, astrocytes, oligodendrocytes, and/or Schwann cells.

In some embodiments, the promoter is a liver promoter (e.g., hAAT, TBG), skeletal muscle specific promoter (e.g., desmin, MCK, C512), B cell promoter, monocyte promoter, leukocyte promoter, macrophage promoter, pancreatic acinar cell promoter, endothelial cell promoter, lung tissue promoter, and/or cardiac or cardiovascular promoter (e.g., aMHC, cTnT, and CMV-MLC2k).

In some embodiments, the promoter is a tissue-specific promoter for payload expression in a cell or tissue of the central nervous system. In some embodiments, the promoter is synapsin (Syn) promoter, glutamate vesicular transporter (VGLUT) promoter, vesicular GABA transporter (VGAT) promoter, parvalbumin (PV) promoter, sodium channel Naᵥ 1.8 promoter, tyrosine hydroxylase (TH) promoter, choline acetyltransferase (ChaT) promoter, methyl-CpG binding protein 2 (MeCP2) promoter, Ca²⁺/calmodulin-dependent protein kinase II (CaMKII) promoter, metabotropic glutamate receptor 2 (mGluR2) promoter, neurofilament light (NFL) or heavy (NFH) promoter, neuron-specific enolase (NSE) promoter, P-globin minigene nP2 promoter, preproenkephalin (PPE) promoter, enkephalin (Enk) promoter, and excitatory amino acid transporter 2 (EAAT2) promoter. In some embodiments, the promoter is a cell-type specific promoter capable of expression in an astrocyte, e.g., a glial fibrillary acidic protein (GFAP) promoter and a EAAT2 promoter. In some embodiments, the promoter is a cell-type specific promoter capable of expression in an oligodendrocyte, e.g., a myelin basic protein (MBP) promoter.

In some embodiments, the promoter is a GFAP promoter. In some embodiments, the promoter is a synapsin (syn or syn1) promoter, or a fragment thereof.

In some embodiments, the promoter comprises an insulin promoter or a fragment thereof.

In some embodiments, the promoter of the viral genome described herein (e.g., comprised within an AAV particle comprising an AAV capsid variant described herein) comprises an EF-1α promoter or variant thereof.

In some embodiments, the subgenomic region, subregion, subgenomic fragment, subfragment, plurality of fragments, AAV expression construct, variant baculovirus genome, and baculovirus expression construct described herein comprises a heterologous nucleotide sequence, e.g., a sequence of interest (e.g., a nucleotide sequence encoding a Rep-coding region, one or more Rep proteins, a VP-coding region, one or more VP proteins, and/or a payload), which is operably linked to one or more promoters. In some embodiments, the heterologous nucleotide sequence is operably linked to a first promoter (e.g., a baculovirus early promoter or a baculovirus early-late promoter) and/or a second promoter (e.g., a baculovirus late promoter or a baculovirus very late promoter). In some embodiments, the heterologous nucleotide sequence is operably linked to a first promoter and a second promoter. In some embodiments, the first and/or second promoter is selected from a baculovirus promoter, a viral promoter, an insect viral promoter, a non-insect viral promoter, a vertebrate viral promoter, a chimeric promoter from one or more species including virus and non-virus elements, a synthetic promoter, or a variant thereof. In some embodiments, the first and/or second promoter chosen from a polh promoter, a p10 promoter, a ctx promoter, a gp64 promoter, an IE promoter, an IE-1 promoter, a p6.9 promoter, a Dmhsp70 promoter, a Hsp70 promoter, a p5 promoter, a p19 promoter, a p35 promoter, a p40 promoter, or a variant, e.g., functional fragment, thereof.

In some embodiments, the first and/or second promoter is selected from an a baculovirus early promoter, baculovirus late promoter, baculovirus early-late promoter, or a baculovirus very late promoter.

In some embodiments, the first and/or second promoter is a baculovirus early-late promoter (e.g., a gp64 promoter). In some embodiments, the first or second promoter is a baculovirus very late promoter (e.g., a polh promoter). In some embodiments, (a) the first promoter is an baculovirus early-late promoter and the second promoter is a baculovirus very late promoter, (b) the first promoter is a baculovirus very late promoter and the second promoter is a baculovirus early-late promoter, (c) the first promoter is a baculovirus early promoter and the second promoter is a baculovirus early-late promoter, (d) the first promoter is a baculovirus early-late promoter and the second promoter is a baculovirus early promoter, (e) the first promoter is a baculovirus early promoter and the second promoter is a baculovirus late promoter, (f) the first promoter is a baculovirus late promoter and the second promoter is a baculovirus early promoter, (g) the first promoter is a baculovirus early-late promoter and the second promoter is a baculovirus late promoter, (h) the first promoter is a baculovirus late promoter and the second promoter is a baculovirus early-late promoter, (i) the first promoter is a baculovirus late promoter and the second promoter is a baculovirus very-late promoter, (j) the first promoter is a baculovirus very-late promoter and the second promoter is a baculovirus late promoter, (k) the first promoter is a baculovirus early promoter and the second promoter is a baculovirus very late promoter, (1) the first promoter is a baculovirus very late promoter and the second promoter is a baculovirus early promoter, (m) the first promoter is a baculovirus early promoter and the second promoter is a baculovirus early promoter, (n) the first promoter is a baculovirus early-late promoter and the second promoter is a baculovirus early-late promoter, or (o) the first promoter is a baculovirus late promoter and the second promoter is a baculovirus late promoter. In some embodiments, the first promoter is a baculovirus early-late promoter (e.g., gp64 promoter) and the second promoter is a baculovirus very late promoter (e.g., polh promoter). In some embodiments, the first promoter is a baculovirus early promoter and the second promoter is a baculovirus late promoter.

In some embodiments, (a) the baculovirus early promoter is selected from: a lef3 promoter, a dbp promoter, a p35 promoter, an orf82 promoter, an get promoter, an orf81 promoter, an orf122 promoter, a pk-2 promoter, an orf55 promoter, an etl promoter, a hcf-1 promoter, an etm promoter, a lef-2 promoter, a lef-6 promoter, an orf84 promoter, an orf118 promoter, or an orf111 promoter, (b) the baculovirus early-late promoter is selected from: a lef2 promoter, a orf13 promoter, a orf23 promoter, a pkip promoter, a v-fgf promoter, a pp31 promoter, an odv-e66 promoter, an orf74 promoter, an orf79 promoter, an orf82 promoter, a p15 promoter, a cg30 promoter, a helicase promoter, an he65 promoter, an orf114 promoter, a pk-2 promoter, a gp64 promoter, a gp16 promoter, an alk-exo promoter, a p35 promoter, a me53 promoter, or an ie0 promoter, (c) the baculovirus late promoter is selected from: a ptpase promoter, an Ac-bro promoter, a ctx promoter, an orf5 promoter, an orf19 promoter, an orf20 promoter, an sod promoter, a HisP promoter, an orf34 promoter, a v-ubi promoter, an orf38 promoter, an orf43 promoter, an orf44 promoter, an orf56 promoter, an orf59 promoter, an orf60 promoter, or an fp-25k promoter, and/or (d) the baculovirus very late promoter is selected from a p10 promoter or a polh promoter.

In some embodiments, the first and/or second promoter comprises a TATA box motif and/or a CAGT motif. In some embodiments, the first and/or second promoter comprises a TAAG motif (e.g., an ATAAG nucleotide sequence). In some embodiments, the first and/or second promoter comprises both a TATA box motif and a TAAG motif. In some embodiments, the first or second promoter comprises a binding site for VLF-1.

In some embodiments, the first or second promoter is a gp64 promoter (e.g., an OpMNPV gp64 promoter). In some embodiments, the first or second promoter is a polh promoter (e.g., an OpMNPV polh promoter or an AcMNPV polh promoter). In some embodiments, the first promoter is a gp64 promoter and the second promoter is a polh promoter. In some embodiments, the first promoter is a polh promoter and the second promoter is a gp64 promoter. In some embodiments, the sequence of interest is operably linked to a first promoter which is a baculovirus early-late promoter and a second promoter which is baculovirus very late promoter, e.g., a gp64 promoter and a polh promoter, optionally, wherein the Rep-coding region is present downstream of a homologous repeat region hr5.

In some embodiments, the first promoter and the second promoter are the same. In some embodiments, the first promoter and the second promoter are different. In some embodiments, the first promoter and the second promoter are each a polh promoter.

In some embodiments, the sequence of interest is operably linked to a gp64 promoter comprising the nucleotide sequence of SEQ ID NO: 217; a nucleotide sequence at least 70%, 75%, 80%, 85%, 90%, 95%, 96%, 97%, 98%, or 99% identical thereto; a nucleotide sequence comprising at least one, two, three, four, five, six, or seven, but no more than ten different nucleotides relative to SEQ ID NO: 217; or a nucleotide sequence comprising at least one, two, three, four, five, six, or seven, but no more than ten modifications (e.g., substitutions) relative to SEQ ID NO: 217.

In some embodiments, the sequence of interest is operably linked to a polh promoter comprising the nucleotide sequence of SEQ ID NO: 167 or 220; a nucleotide sequence at least 70%, 75%, 80%, 85%, 90%, 95%, 96%, 97%, 98%, or 99% identical thereto; a nucleotide sequence comprising at least one, two, three, four, five, six, or seven, but no more than ten different nucleotides relative to SEQ ID NO: 167 or 220; or a nucleotide sequence comprising at least one, two, three, four, five, six, or seven, but no more than ten modifications (e.g., substitutions) relative to SEQ ID NO: 167 or 220.

In some embodiments, the sequence of interest is operably linked to a first promoter and the second promoter comprising the nucleotide sequence of SEQ ID NO: 221; a nucleotide sequence at least 70%, 75%, 80%, 85%, 90%, 95%, 96%, 97%, 98%, or 99% identical thereto; a nucleotide sequence comprising at least one, two, three, four, five, six, or seven, but no more than ten different nucleotides relative to SEQ ID NO: 221; or a nucleotide sequence comprising at least one, two, three, four, five, six, or seven, but no more than ten modifications (e.g., substitutions) relative to SEQ ID NO: 221.

### Viral Genome Component: Untranslated Regions (UTRs)

In some embodiments, wild type untranslated regions (UTRs) of a gene are transcribed but not translated. Generally, the 5′ UTR starts at the transcription start site and ends at the start codon and the 3′ UTR starts immediately following the stop codon and continues until the termination signal for transcription.

Features typically found in abundantly expressed genes of specific target organs (e.g., CNS tissue, muscle, or DRG) may be engineered into UTRs to enhance stability and protein production. As a non-limiting example, a 5′ UTR from mRNA normally expressed in the brain (e.g., huntingtin) may be used in the viral genomes of the AAV particles described herein to enhance expression in neuronal cells or other cells of the central nervous system.

While not wishing to be bound by theory, wild-type 5′ untranslated regions (UTRs) include features which play roles in translation initiation. Kozak sequences, which are commonly known to be involved in the process by which the ribosome initiates translation of many genes, are usually included in 5′ UTRs. Kozak sequences have the consensus CCR(A/G)CCAUGG, where R is a purine (adenine or guanine) three bases upstream of the start codon (ATG), which is followed by another ‘G’.

In one embodiment, the 5′UTR in the viral genome includes a Kozak sequence.

In one embodiment, the 5′UTR in the viral genome does not include a Kozak sequence.

While not wishing to be bound by theory, wild-type 3′ UTRs are known to have stretches of Adenosines and Uridines embedded therein. These AU rich signatures are particularly prevalent in genes with high rates of turnover. Based on their sequence features and functional properties, the AU rich elements (AREs) can be separated into three classes (Chen et al, 1995, the contents of which are herein incorporated by reference in its entirety): Class I AREs, such as, but not limited to, c-Myc and MyoD, contain several dispersed copies of an AUUUA motif within U-rich regions. Class II AREs, such as, but not limited to, GM-CSF and TNF-α, possess two or more overlapping UUAUUUA(U/A)(U/A) nonamers. Class III ARES, such as, but not limited to, c-Jun and Myogenin, are less well defined. These U rich regions do not contain an AUUUA motif. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′ UTR AU rich elements (AREs) can be used to modulate the stability of a polynucleotide. When engineering specific polynucleotides, e.g., payload regions of viral genomes, one or more copies of an ARE can be introduced to make polynucleotides less stable and thereby curtail translation and decrease production of the resultant protein. Likewise, AREs can be identified and removed or mutated to increase the intracellular stability and thus increase translation and production of the resultant protein.

In one embodiment, the 3′ UTR of the viral genome may include an oligo(dT) sequence for templated addition of a poly-A tail.

In one embodiment, the viral genome may include at least one miRNA seed, binding site or full sequence. microRNAs (or miRNA or miR) are 19-25 nucleotide noncoding RNAs that bind to the sites of nucleic acid targets and down-regulate gene expression either by reducing nucleic acid molecule stability or by inhibiting translation. In some embodiments, a microRNA sequence comprises a seed region, e.g., a sequence in the region of positions 2-8 of the mature microRNA, which has Watson-Crick sequence fully or partially complementarity to the miRNA target sequence of the nucleic acid.

In one embodiment, the viral genome may be engineered to include, alter or remove at least one miRNA binding site, full sequence or seed region.

Any UTR from any gene known in the art may be incorporated into the viral genome of the AAV particle. These UTRs, or portions thereof, may be placed in the same orientation as in the gene from which they were selected or they may be altered in orientation or location. In one embodiment, the UTR used in the viral genome of the AAV particle may be inverted, shortened, lengthened, made with one or more other 5′ UTRs or 3′ UTRs known in the art. As used herein, the term “altered” as it relates to a UTR, means that the UTR has been changed in some way in relation to a reference sequence. For example, a 3′ or 5′ UTR may be altered relative to a wild type or native UTR by the change in orientation or location as taught above or may be altered by the inclusion of additional nucleotides, deletion of nucleotides, swapping or transposition of nucleotides.

In one embodiment, the viral genome of the AAV particle comprises at least one artificial UTR which is not a variant of a wild type UTR.

In one embodiment, the viral genome of the AAV particle comprises UTRs which have been selected from a family of transcripts whose proteins share a common function, structure, feature or property.

### Viral Genome Component: Polyadenylation Sequence

In some embodiments, the viral genome of the AAV particle described herein (e.g., an AAV particle comprising an AAV capsid polypeptide) comprises a polyadenylation sequence. In some embodiments, the viral genome of the AAV particle (e.g., an AAV particle comprising an AAV capsid polypeptide) comprises a polyadenylation sequence between the 3′ end of the nucleotide sequence encoding the payload and the 5′ end of the 3′ITR.

### Viral Genome Component: Introns

In some embodiments, the viral genome of the AAV particle as described herein (e.g., an AAV particle comprising an AAV capsid polypeptide) comprises an element to enhance the payload target specificity and expression (See e.g., Powell et al. Viral Expression Cassette Elements to Enhance Transgene Target Specificity and Expression in Gene Therapy, Discov. Med, 2015, 19(102): 49-57; the contents of which are herein incorporated by reference in their entirety) such as an intron. Non-limiting examples of introns include, MVM (67-97 bps), F.IX truncated intron 1 (300 bps), P-globin SD/immunoglobulin heavy chain splice acceptor (250 bps), adenovirus splice donor/immunoglobin splice acceptor (500 bps), SV40 late splice donor/splice acceptor (19S/16S) (180 bps) and hybrid adenovirus splice donor/IgG splice acceptor (230 bps).

### Viral Genome Component: Stuffer Sequences

In some embodiments, the viral genome of an AAV particle described herein (e.g., an AAV particle comprising an AAV capsid polypeptide), comprises an element to improve packaging efficiency and expression, such as a stuffer or filler sequence. Non-limiting examples of stuffer sequences include albumin and/or alpha-1 antitrypsin. Any known viral, mammalian, or plant sequence may be manipulated for use as a stuffer sequence.

In one embodiment, the stuffer or filler sequence may be from about 100-3500 nucleotides in length. The stuffer sequence may have a length of about 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900 or 3000 nucleotides.

Viral Genome Component: miRNA

In some embodiments, the viral genome of an AAV particle described herein (e.g., an AAV particle comprising an AAV capsid polypeptide) comprises a sequence encoding a miRNA to reduce the expression of the payload in a tissue or cell, e.g., the DRG (dorsal root ganglion), or neurons of other ganglia, such as those of the sympathetic or parasympathetic nervous system. In some embodiments, a miRNA, e.g., a miR183, a miR182, and/or miR96, may be encoded in the viral genome to modulate, e.g., reduce the expression, of the viral genome in a DRG neuron. As another non-limiting example, a miR-122 miRNA may be encoded in the viral genome to modulate, e.g., reduce, the expression of the viral genome in the liver. In some embodiments, a miRNA, e.g., a miR-142-3p, may be encoded in the viral genome to modulate, e.g., reduce, the expression, of the viral genome in a cell or tissue of the hematopoietic lineage, including for example immune cells (e.g., antigen presenting cells or APC, including dendritic cells (DCs), macrophages, and B-lymphocytes). In some embodiments, a miRNA, e.g., a miR-1, may be encoded in the viral genome to modulate, e.g., reduce, the expression, of the viral genome in a cell or tissue of the heart.

### Viral Genome Component: miR Binding Site

Tissue- or cell-specific expression of the AAV viral particles disclosed herein can be enhanced by introducing tissue- or cell-specific regulatory sequences, e.g., promoters, enhancers, microRNA binding sites, e.g., a detargeting site. Without wishing to be bound by theory, it is believed that an encoded miR binding site can modulate, e.g., prevent, suppress, or otherwise inhibit, the expression of a gene of interest on the viral genome disclosed herein, based on the expression of the corresponding endogenous microRNA (miRNA) or a corresponding controlled exogenous miRNA in a tissue or cell, e.g., a non-targeting cell or tissue. In some embodiments, a miR binding site modulates, e.g., reduces, expression of the payload encoded by a viral genome of an AAV particle described herein in a cell or tissue where the corresponding mRNA is expressed.

In some embodiments, the viral genome of an AAV particle described herein comprises a nucleotide sequence encoding a microRNA binding site, e.g., a detargeting site. In some embodiments, the viral genome of an AAV particle described herein comprises a nucleotide sequence encoding a miR binding site, a microRNA binding site series (miR BSs), or a reverse complement thereof.

In some embodiments, the nucleotide sequence encoding the miR binding site series or the miR binding site is located in the 3′-UTR region of the viral genome (e.g., 3′ relative to the nucleotide sequence encoding a payload), e.g., before the polyA sequence, 5′-UTR region of the viral genome (e.g., 5′ relative to the nucleotide sequence encoding a payload), or both.

In some embodiments, the encoded miR binding site series comprise at least 1-5 copies, e.g., at least 1-3, 2-4, 3-5, 1, 2, 3, 4, 5 or more copies of a miR binding site (miR BS). In some embodiments, all copies are identical, e.g., comprise the same miR binding site. In some embodiments, the miR binding sites within the encoded miR binding site series are continuous and not separated by a spacer. In some embodiments, the miR binding sites within an encoded miR binding site series are separated by a spacer, e.g., a non-coding sequence. In some embodiments, the spacer is about 1 to 6 nucleotides or about 5 to 10 nucleotides, e.g., about 7-8 nucleotides, nucleotides in length. In some embodiments, the spacer coding sequence or reverse complement thereof comprises one or more of (i) GGAT; (ii) CACGTG; (iii) GCATGC, or a repeat of one or more of (i)-(iii). In some embodiments, the spacer comprises the nucleotide sequence of GATAGTTA (SEQ ID NO: 1), or a nucleotide sequence having at least one, two, or three modifications, e.g., substitutions (e.g., conservative substitutions), insertions, or deletions, but no more than four modifications, e.g., substitutions (e.g., conservative substitutions), insertions, or deletions, of GATAGTTA (SEQ ID NO: 1).

In some embodiments, the encoded miR binding site series comprise at least 1-5 copies, e.g., at least 1-3, 2-4, 3-5, 1, 2, 3, 4, 5 or more copies of a miR binding site (miR BS). In some embodiments, at least 1, 2, 3, 4, 5, or all of the copies are different, e.g., comprise a different miR binding site. In some embodiments, the miR binding sites within the encoded miR binding site series are continuous and not separated by a spacer. In some embodiments, the miR binding sites within an encoded miR binding site series are separated by a spacer, e.g., a non-coding sequence. In some embodiments, the spacer is about 1 to 6 nucleotides or about 5 to 10 nucleotides, e.g., about 7-8 nucleotides, in length. In some embodiments, the spacer comprises one or more of (i) GGAT; (ii) CACGTG; (iii) GCATGC, or a repeat of one or more of (i)-(iii). In some embodiments, the spacer comprises the nucleotide sequence of GATAGTTA (SEQ ID NO: 1), or a nucleotide sequence having at least one, two, or three modifications, e.g., substitutions (e.g., conservative substitutions), insertions, or deletions, but no more than four modifications, e.g., substitutions (e.g., conservative substitutions), insertions, or deletions, of GATAGTTA (SEQ ID NO: 1).

In some embodiments, the encoded miR binding site is substantially identical (e.g., at least 70%, 75%, 80%, 85%, 90%, 95%, 99% or 100% identical), to the miR in the host cell. In some embodiments, the encoded miR binding site comprises at least 1, 2, 3, 4, or 5 mismatches or no more than 6, 7, 8, 9, or 10 mismatches to a miR in the host cell. In some embodiments, the mismatched nucleotides are contiguous. In some embodiments, the mismatched nucleotides are non-contiguous. In some embodiments, the mismatched nucleotides occur outside the seed region-binding sequence of the miR binding site, such as at one or both ends of the miR binding site. In some embodiments, the miR binding site is 100% identical to the miR in the host cell.

In some embodiments, the nucleotide sequence encoding the miR binding site is substantially complimentary (e.g., at least 70%, 75%, 80%, 85%, 90%, 95%, 99% or 100% complimentary), to the miR in the host cell. In some embodiments, to complementary sequence of the nucleotide sequence encoding the miR binding site comprises at least 1, 2, 3, 4, or 5 mismatches or no more than 6, 7, 8, 9, or 10 mismatches to a miR in the host cell. In some embodiments, the mismatched nucleotides are contiguous. In some embodiments, the mismatched nucleotides are non-contiguous. In some embodiments, the mismatched nucleotides occur outside the seed region-binding sequence of the miR binding site, such as at one or both ends of the miR binding site. In some embodiments, the encoded miR binding site is 100% complimentary to the miR in the host cell.

In some embodiments, an encoded miR binding site or sequence region is at least about 10 to about 125 nucleotides in length, e.g., at least about 10 to 50 nucleotides, 10 to 100 nucleotides, 50 to 100 nucleotides, 50 to 125 nucleotides, or 100 to 125 nucleotides in length. In some embodiments, an encoded miR binding site or sequence region is at least about 7 to about 28 nucleotides in length, e.g., at least about 8-28 nucleotides, 7-28 nucleotides, 8-18 nucleotides, 12-28 nucleotides, 20-26 nucleotides, 22 nucleotides, 24 nucleotides, or 26 nucleotides in length, and optionally comprises at least one consecutive region (e.g., 7 or 8 nucleotides) complementary (e.g., fully or partially complementary) to the seed sequence of a miRNA (e.g., a miR122, a miR142, a miR183, or a miR1).

In some embodiments, the encoded miR binding site is complementary (e.g., fully or partially complementary) to a miR expressed in liver or hepatocytes, such as miR122. In some embodiments, the encoded miR binding site or encoded miR binding site series comprises a miR122 binding site sequence. In some embodiments, the encoded miR122 binding site comprises the nucleotide sequence of ACAAACACCATTGTCACACTCCA (SEQ ID NO: 2), or a nucleotide sequence having at least 50%, 55%, 60%, 65%, 70%, 75%, 80%, 85%, 90%, at least 95%, at least 99%, or 100% sequence identity, or having at least one, two, three, four, five, six, or seven modifications, e.g., substitutions (e.g., conservative substitutions), insertions, or deletions, but no more than ten modifications, e.g., substitutions (e.g., conservative substitutions), insertions, or deletions, to SEQ ID NO: 2, e.g., wherein the modification can result in a mismatch between the encoded miR binding site and the corresponding miRNA. In some embodiments, the viral genome comprises at least 2, 3, 4, or 5 copies of the encoded miR122 binding site, e.g., an encoded miR122 binding site series, optionally wherein the encoded miR122 binding site series comprises the nucleotide sequence of: ACAAACACCATTGTCACACTCCACACAAACACCATTGTCACACTCCACACAAACACCATTGTCACACTCC A (SEQ ID NO: 3), or a nucleotide sequence having at least 50%, 55%, 60%, 65%, 70%, 75%, 80%, 85%, 90%, at least 95%, at least 99%, or 100% sequence identity, or having at least one, two, three, four, five, six, or seven modifications, e.g., substitutions (e.g., conservative substitutions), insertions, or deletions, but no more than ten modifications, e.g., substitutions (e.g., conservative substitutions), insertions, or deletions, to SEQ ID NO: 3, e.g., wherein the modification can result in a mismatch between the encoded miR binding site and the corresponding miRNA. In some embodiments, at least two of the encoded miR122 binding sites are connected directly, e.g., without a spacer. In other embodiments, at least two of the encoded miR122 binding sites are separated by a spacer, e.g., 1, 2, 3, 4, 5, 6, 7, 8, 9, or 10 nucleotides in length, which is located between two or more consecutive encoded miR122 binding site sequences. In embodiments, the spacer is about 1 to 6 nucleotides or about 5 to 10 nucleotides, e.g., about 7-8, in length. In some embodiments, the spacer coding sequence or reverse complement thereof comprises one or more of (i) GGAT; (ii) CACGTG; (iii) GCATGC, or a repeat of one or more of (i)-(iii). In some embodiments, an encoded miR binding site series comprises at least 3-5 copies (e.g., 4 copies) of a miR122 binding site, with or without a spacer, wherein the spacer is about 1 to 6 nucleotides or about 5 to 10 nucleotides, e.g., about 7-8 nucleotides or about 8 nucleotides, in length. In some embodiments, the spacer comprises the nucleotide sequence of GATAGTTA (SEQ ID NO: 1), or a nucleotide sequence having at least one, two, or three modifications, e.g., substitutions (e.g., conservative substitutions), insertions, or deletions, but no more than four modifications, e.g., substitutions (e.g., conservative substitutions), insertions, or deletions, of GATAGTTA (SEQ ID NO: 1).

In some embodiments, the encoded miR binding site is complementary (e.g., fully or partially complementary) to a miR expressed in the heart. In embodiments, the encoded miR binding site or encoded miR binding site series comprises a miR-1 binding site. In some embodiments, the encoded miR-1 binding site comprises the nucleotide sequence of ATACATACTTCTTTACATTCCA (SEQ ID NO: 4), a nucleotide sequence having at least 50%, 55%, 60%, 65%, 70%, 75%, 80%, 85%, 90%, at least 95%, at least 99%, or 100% sequence identity, or having at least one, two, three, four, five, six, or seven modifications e.g., substitutions (e.g., conservative substitutions), insertions, or deletions, but no more than ten modifications, e.g., substitutions (e.g., conservative substitutions), insertions, or deletions, to SEQ ID NO: 4, e.g., wherein the modification can result in a mismatch between the encoded miR binding site and the corresponding miRNA. In some embodiments, the viral genome comprises at least 2, 3, 4, or 5 copies of the encoded miR-1 binding site, e.g., an encoded miR-1 binding site series. In some embodiments, the at least 2, 3, 4, or 5 copies (e.g., 2 or 3 copies) of the encoded miR-1 binding site are continuous (e.g., not separated by a spacer) or separated by a spacer. In some embodiments, the spacer is about 1 to 6 nucleotides or about 5 to 10 nucleotides, e.g., about 7-8 nucleotides or about 8 nucleotides, in length. In some embodiments, the spacer sequence comprises one or more of (i) GGAT; (ii) CACGTG; (iii) GCATGC, or a repeat of one or more of (i)-(iii). In some embodiments, the spacer comprises the nucleotide sequence of GATAGTTA (SEQ ID NO: 1), or a nucleotide sequence having at least one, two, or three modifications, e.g., substitutions (e.g., conservative substitutions), insertions, or deletions, but no more than four modifications, e.g., substitutions (e.g., conservative substitutions), insertions, or deletions, of GATAGTTA (SEQ ID NO: 1).

In some embodiments, the encoded miR binding site is complementary (e.g., fully or partially complementary) to a miR expressed in hematopoietic lineage, including immune cells (e.g., antigen presenting cells or APC, including dendritic cells (DCs), macrophages, and B-lymphocytes). In some embodiments, the encoded miR binding site complementary to a miR expressed in hematopoietic lineage comprises a nucleotide sequence disclosed, e.g., in US 2018/0066279, the contents of which are incorporated by reference herein in its entirety.

In some embodiments, the encoded miR binding site or encoded miR binding site series comprises a miR-142-3p binding site sequence. In some embodiments, the encoded miR-142-3p binding site comprises the nucleotide sequence of TCCATAAAGTAGGAAACACTACA (SEQ ID NO: 5), a nucleotide sequence having at least 50%, 55%, 60%, 65%, 70%, 75%, 80%, 85%, 90%, at least 95%, at least 99%, or 100% sequence identity, or having at least one, two, three, four, five, six, or seven modifications e.g., substitutions (e.g., conservative substitutions), insertions, or deletions, but no more than ten modifications, e.g., substitutions (e.g., conservative substitutions), insertions, or deletions, to SEQ ID NO: 5, e.g., wherein the modification can result in a mismatch between the encoded miR binding site and the corresponding miRNA. In some embodiments, the viral genome comprises at least 2, 3, 4, or 5 copies of the encoded miR-142-3p binding site, e.g., an encoded miR-142-3p binding site series. In some embodiments, the at least 2, 3, 4, or 5 copies (e.g., 2 or 3 copies) of the encoded miR-142-3p binding site are continuous (e.g., not separated by a spacer) or separated by a spacer. In some embodiments, the spacer is about 1 to 6 nucleotides or about 5 to 10 nucleotides, e.g., about 7-8 nucleotides or about 8 nucleotides, in length. In some embodiments, the spacer sequence comprises one or more of (i) GGAT; (ii) CACGTG; (iii) GCATGC, or a repeat of one or more of (i)-(iii). In some embodiments, the spacer comprises the nucleotide sequence of GATAGTTA (SEQ ID NO: 1), or a nucleotide sequence having at least one, two, or three modifications, e.g., substitutions (e.g., conservative substitutions), insertions, or deletions, but no more than four modifications, e.g., substitutions (e.g., conservative substitutions), insertions, or deletions, of GATAGTTA (SEQ ID NO: 1).

In some embodiments, the encoded miR binding site is complementary (e.g., fully complementary or partially complementary) to a miR expressed in a DRG (dorsal root ganglion) neuron, e.g., a miR183, a miR182, and/or miR96 binding site. In some embodiments, the encoded miR binding site is complementary to a miR expressed in expressed in a DRG neuron comprises a nucleotide sequence disclosed, e.g., in WO2020/132455, the contents of which are incorporated by reference herein in its entirety.

In some embodiments, the encoded miR binding site or encoded miR binding site series comprises a miR183 binding site sequence. In some embodiments, the encoded miR183 binding site comprises the nucleotide sequence of

(SEQ ID NO: 6)
AGTGAATTCTACCAGTGCCATA,

or a nucleotide sequence having at least 50%, 55%, 60%, 65%, 70%, 75%, 80%, 85%, 90%, at least 95%, at least 99%, or 100% sequence identity, or having at least one, two, three, four, five, six, or seven modifications, e.g., substitutions (e.g., conservative substitutions), insertions, or deletions, but no more than ten modifications, e.g., substitutions (e.g., conservative substitutions), insertions, or deletions, to SEQ ID NO: 6, e.g., wherein the modification can result in a mismatch between the encoded miR binding site and the corresponding miRNA. In some embodiments, the sequence complementary to the seed sequence corresponds to the double underlined of the encoded miR-183 binding site sequence. In some embodiments, the viral genome comprises at least comprises at least 2, 3, 4, or 5 copies (e.g., at least 2 or 3 copies) of the encoded miR183 binding site, e.g., an encoded miR183 binding site. In some embodiments, the at least 2, 3, 4, or 5 copies (e.g., 2 or 3 copies) of the encoded miR183 binding site are continuous (e.g., not separated by a spacer) or separated by a spacer. In some embodiments, the spacer is about 1 to 6 nucleotides or about 5 to 10 nucleotides, e.g., about 7-8 nucleotides or about 8 nucleotides, in length. In some embodiments, the spacer comprises the nucleotide sequence of GATAGTTA (SEQ ID NO: 1), or a nucleotide sequence having at least one, two, or three modifications, e.g., substitutions (e.g., conservative substitutions), insertions, or deletions, but no more than four modifications, e.g., substitutions (e.g., conservative substitutions), insertions, or deletions, of GATAGTTA (SEQ ID NO: 1). In some embodiments, the spacer sequence comprises one or more of (i) GGAT; (ii) CACGTG; (iii) GCATGC, or a repeat of one or more of (i)-(iii).

In some embodiments, the encoded miR binding site or the encoded miR binding site series comprises a miR182 binding site sequence. In some embodiments, the encoded miR182 binding site comprises, the nucleotide sequence of AGTGTGAGTTCTACCATTGCCAAA (SEQ ID NO: 7), a sequence having at least 50%, 55%, 60%, 65%, 70%, 75%, 80%, 85%, 90%, at least 95%, at least 99%, or 100% sequence identity, or having at least one, two, three, four, five, six, or seven modifications, e.g., substitutions (e.g., conservative substitutions), insertions, or deletions, but no more than ten modifications, e.g., substitutions (e.g., conservative substitutions), insertions, or deletions, to SEQ ID NO: 7, e.g., wherein the modification can result in a mismatch between the encoded miR binding site and the corresponding miRNA. In some embodiments, the viral genome comprises at least 2, 3, 4, or 5 copies of the encoded miR182 binding site, e.g., an encoded miR182 binding site series. In some embodiments, the at least 2, 3, 4, or 5 copies (e.g., 2 or 3 copies) of the encoded miR182 binding site are continuous (e.g., not separated by a spacer) or separated by a spacer. In some embodiments, the spacer is about 1 to 6 nucleotides or about 5 to 10 nucleotides, e.g., about 7-8 nucleotides or about 8 nucleotides, in length. In some embodiments, the spacer comprises the nucleotide sequence of GATAGTTA (SEQ ID NO: 1), or a nucleotide sequence having at least one, two, or three modifications, e.g., substitutions (e.g., conservative substitutions), insertions, or deletions, but no more than four modifications, e.g., substitutions (e.g., conservative substitutions), insertions, or deletions, of GATAGTTA (SEQ ID NO: 1). In some embodiments, the spacer sequence comprises one or more of (i) GGAT; (ii) CACGTG; (iii) GCATGC, or a repeat of one or more of (i)-(iii).

In some embodiments, the encoded miR binding site or the encoded miR binding site series comprises a miR96 binding site sequence. In some embodiments, the encoded miR96 binding site comprises the nucleotide sequence of AGCAAAAATGTGCTAGTGCCAAA (SEQ ID NO: 8), a sequence having at least 50%, 55%, 60%, 65%, 70%, 75%, 80%, 85%, 90%, at least 95%, at least 99%, or 100% sequence identity, or having at least one, two, three, four, five, six, or seven modifications, e.g., substitutions (e.g., conservative substitutions), insertions, or deletions, but no more than ten modifications, e.g., substitutions (e.g., conservative substitutions), insertions, or deletions, to SEQ ID NO: 8, e.g., wherein the modification can result in a mismatch between the encoded miR binding site and the corresponding miRNA. In some embodiments, the viral genome comprises at least 2, 3, 4, or 5 copies of the encoded miR96 binding site, e.g., an encoded miR96 binding site series. In some embodiments, the at least 2, 3, 4, or 5 copies (e.g., 2 or 3 copies) of the encoded miR96 binding site are continuous (e.g., not separated by a spacer) or separated by a spacer. In some embodiments, the spacer is about 1 to 6 nucleotides or about 5 to 10 nucleotides, e.g., about 7-8 nucleotides or about 8 nucleotides, in length. In some embodiments, the spacer comprises the nucleotide sequence of GATAGTTA (SEQ ID NO: 1), or a nucleotide sequence having at least one, two, or three modifications, e.g., substitutions (e.g., conservative substitutions), insertions, or deletions, but no more than four modifications, e.g., substitutions (e.g., conservative substitutions), insertions, or deletions, of GATAGTTA (SEQ ID NO: 1). In some embodiments, the spacer sequence comprises one or more of (i) GGAT; (ii) CACGTG; (iii) GCATGC, or a repeat of one or more of (i)-(iii).

In some embodiments, the encoded miR binding site series comprises a miR122 binding site, a miR-1, a miR142 binding site, a miR183 binding site, a miR182 binding site, a miR 96 binding site, or a combination thereof. In some embodiments, the encoded miR binding site series comprises at least 2, 3, 4, or 5 copies of a miR122 binding site, a miR142 binding site, a miR183 binding site, a miR182 binding site, a miR 96 binding site, or a combination thereof. In some embodiments, at least two of the encoded miR binding sites are connected directly, e.g., without a spacer. In other embodiments, at least two of the encoded miR binding sites are separated by a spacer, e.g., 1, 2, 3, 4, 5, 6, 7, 8, 9, or 10 nucleotides in length, which is located between two or more consecutive encoded miR binding site sequences. In embodiments, the spacer is at least about 5 to 10 nucleotides, e.g., about 7-8 nucleotides or about 8 nucleotides, in length. In some embodiments, the spacer coding sequence or reverse complement thereof comprises one or more of (i) GGAT; (ii) CACGTG; (iii) GCATGC, or a repeat of one or more of (i)-(iii). In some embodiments, the spacer comprises the nucleotide sequence of GATAGTTA (SEQ ID NO: 1), or a nucleotide sequence having at least one, two, or three modifications, e.g., substitutions (e.g., conservative substitutions), insertions, or deletions, but no more than four modifications, e.g., substitutions (e.g., conservative substitutions), insertions, or deletions, of GATAGTTA (SEQ ID NO: 1).

In some embodiments, an encoded miR binding site series comprises at least 2-5 copies (e.g., 2 or 3 copies) of a combination of at least two, three, four, five, or all of a miR-1, miR122 binding site, a miR142 binding site, a miR183 binding site, a miR182 binding site, a miR96 binding site, wherein each of the miR binding sites within the series are continuous (e.g., not separated by a spacer) or are separated by a spacer. In some embodiments, the spacer is about 1 to 6 nucleotides or about 5 to 10 nucleotides, e.g., about 7-8 nucleotides or about 8 nucleotides, in length. In some embodiments, the spacer sequence comprises one or more of (i) GGAT; (ii) CACGTG; (iii) GCATGC, or a repeat of one or more of (i)-(iii). In some embodiments, the spacer comprises the nucleotide sequence of GATAGTTA (SEQ ID NO: 1), or a nucleotide sequence having at least one, two, or three modifications, e.g., substitutions (e.g., conservative substitutions), insertions, or deletions, but no more than four modifications, e.g., substitutions (e.g., conservative substitutions), insertions, or deletions, of GATAGTTA (SEQ ID NO: 1).

In some embodiments, an encoded miR binding site series comprises at least 2-5 copies (e.g., 2 or 3 copies) of a combination of a miR-122 binding site and a miR-1 binding site, wherein each of the miR binding sites within the series are continuous (e.g., not separated by a spacer) or are separated by a spacer. In some embodiments, the spacer is about 1 to 6 nucleotides or about 5 to 10 nucleotides, e.g., about 7-8 nucleotides or about 8 nucleotides, in length. In some embodiments, the spacer sequence comprises one or more of (i) GGAT; (ii) CACGTG; (iii) GCATGC, or a repeat of one or more of (i)-(iii). In some embodiments, the spacer comprises the nucleotide sequence of GATAGTTA (SEQ ID NO: 1), or a nucleotide sequence having at least one, two, or three modifications, e.g., substitutions (e.g., conservative substitutions), insertions, or deletions, but no more than four modifications, e.g., substitutions (e.g., conservative substitutions), insertions, or deletions, of GATAGTTA (SEQ ID NO: 1).

### Genome Size

In one embodiment, the AAV particle described herein (e.g., an AAV particle comprising an AAV capsid polypeptide) may comprise a single-stranded or double-stranded viral genome. The size of the viral genome may be small, medium, large or the maximum size. As described above, the viral genome may comprise a promoter and a polyA tail.

In one embodiment, the viral genome is a small single stranded viral genome. A small single stranded viral genome may be 2.1 to 3.5 kb in size such as, but not limited to, about 2.1, 2.2, 2.3, 2.4, 2.5, 2.6, 2.7, 2.8, 2.9, 3.0, 3.1, 3.2, 3.3, 3.4, and 3.5 kb in size.

In one embodiment, the viral genome may be a small double stranded viral genome. A small double stranded viral genome may be 1.3 to 1.7 kb in size such as, but not limited to, about 1.3, 1.4, 1.5, 1.6, and 1.7 kb in size.

In one embodiment, the viral genome may be a medium single stranded viral genome. A medium single stranded viral genome may be 3.6 to 4.3 kb in size such as, but not limited to, about 3.6, 3.7, 3.8, 3.9, 4.0, 4.1, 4.2 and 4.3 kb in size.

In one embodiment, the viral genome may be a medium double stranded viral genome. A medium double stranded viral genome may be 1.8 to 2.1 kb in size such as, but not limited to, about 1.8, 1.9, 2.0, and 2.1 kb in size.

In one embodiment, the viral genome may be a large single stranded viral genome. A large single stranded viral genome may be 4.4 to 6.0 kb in size such as, but not limited to, about 4.4, 4.5, 4.6, 4.7, 4.8, 4.9, 5.0, 5.1, 5.2, 5.3, 5.4, 5.5, 5.6, 5.7, 5.8, 5.9 and 6.0 kb in size.

In one embodiment, the viral genome may be a large double stranded viral genome. A large double stranded viral genome may be 2.2 to 3.0 kb in size such as, but not limited to, about 2.2, 2.3, 2.4, 2.5, 2.6, 2.7, 2.8, 2.9 and 3.0 kb in size.

### Payloads

In some embodiments, an AAV particle described herein (e.g. an AAV particle comprising an AAV capsid polypeptide) comprises a viral genome comprising a nucleic acid encoding a payload. In some embodiments, the encoded payload is an RNAi agent or a polypeptide. A payload of the present disclosure may be, but is not limited to, a peptide, a polypeptide, a protein, an antibody, an RNAi agent, etc.

In some embodiments, the nucleotide sequence encoding a payload may comprise a combination of coding and non-coding nucleic acid sequences. In some embodiments, the nucleotide sequence encoding the payload may encode a coding or non-coding RNA.

In some embodiments, the AAV particles described herein, e.g., an AAV particle comprising an AAV capsid polypeptide, e.g., an AAV capsid variant, comprises a nucleic acid encoding a payload. In some embodiments, the encoded payload comprises a therapeutic protein, an antibody, an enzyme, one or more components of a genome editing system, and/or an RNAi agent (e.g., a dsRNA, siRNA, shRNA, pre-miRNA, pri-miRNA, miRNA, stRNA, lncRNA, piRNA, or snoRNA). In some embodiments, the encoded payload modulates, e.g., increases or decreases, the presence, level, and/or activity of a gene, mRNA, protein, or a combination thereof, e.g., in a cell or a tissue.

### Polypeptides

In some embodiments, the encoded payload of AAV particle comprising an AAV capsid polypeptide described herein comprises a polypeptide, protein, or peptide, e.g., a polypeptide, protein, or peptide described herein. The nucleic acid encoding the payload, may encode a product of any known gene and/or a recombinant version thereof. In some embodiments, the nucleic acid encoding the payload may encode at least one allele of apolipoprotein E (APOE) such as, but not limited to ApoE2, ApoE3 and/or ApoE4. In one embodiment, the nucleic acid encoding the payload encodes ApoE2 (cys112, cys158) protein or a fragment or variant thereof. In one embodiment, the nucleic acid encoding the payload encodes an ApoE3 (cys112, arg158) protein or fragment or variant thereof. In one embodiment, the nucleic acid encoding the payload encodes ApoE4 (arg112, arg158). As another non-limiting example, the encoded payload comprises an aromatic L-amin acid decarboxylase (AADC) protein. As another non-limiting example, the encoded payload comprises an antibody, or a fragment thereof. As another non-limiting example, the encoded payload comprises a human survival of motor neuron (SMN) 1 or SMN2 protein, or fragments or variants thereof. As another non-limiting example, the encoded payload region comprises a glucocerebrosidase (GBA1) protein, or a fragment or variant thereof. As another non-limiting example, the encoded payload comprises a granulin precursor or progranulin (GRN) protein, or a fragment or variant thereof. As another non-limiting example, the encoded payload comprises an aspartoacylase (ASPA) protein, or a fragment or variant thereof. As another non-limiting example, the encoded payload comprises a tripeptidyl peptidase I (CLN2) protein, or a fragment or variant thereof. As another non-limiting example, the encoded payload comprises a beta-galactosidase (GLB1) protein, or a fragment or variant thereof. As another non-limiting example, the encoded payload comprises a N-sulphoglucosamine sulphohydrolase (SGSH) protein, or a fragment or variant thereof. As another non-limiting example, the encoded payload comprises an N-acetyl-alpha-glucosaminidase (NAGLU) protein, or a fragment or variant thereof. As another non-limiting example, the encoded payload comprises an iduronate 2-sulfatase (IDS) protein, or a fragment or variant thereof. As another non-limiting example, the encoded payload comprises an intracellular cholesterol transporter (NPC1) protein, or a fragment or variant thereof. As another non-limiting example, the encoded payload comprises a gigaxonin (GAN) protein, or a fragment or variant thereof. The AAV viral genomes encoding polypeptides described herein may be useful in the fields of human disease, viruses, infections veterinary applications and a variety of in vivo and in vitro settings.

Amino acid sequences of a payload polypeptide encoded by a viral genome described herein, may be translated as a whole polypeptide, a plurality of polypeptides or fragments of polypeptides, which independently may be encoded by one or more nucleic acids, fragments of nucleic acids or variants of any of the aforementioned.

### Antibodies and Antibody Binding Fragments

In some embodiments, the encoded payload of AAV particle comprising an AAV capsid variant described herein comprises an antibody or antibody binding fragment. In some embodiments, the antibody may be a full antibody, a fragment, or any functional variant thereof. As non-limiting examples, an antibody may be a native antibody (e.g., with two heavy and two light chains), a heavy chain variable region, a light chain variable region, a heavy chain constant region, a light chain constant region, Fab, Fab′, F(ab′)₂, Fv, or scFv fragments, a diabody, a linear antibody, a single-chain antibody, a multi-specific antibody, an intrabody, one or more heavy chain complementarity determining regions (CDR), one or more light chain CDRs, a bi-specific antibody, a monoclonal antibody, a polyclonal antibody, a humanized antibody, an antibody mimetic, an antibody variant, a miniaturized antibody, a unibody, a maxibody, and/or a chimeric antigen receptor. The encoded antibody or antibody binding fragment may be useful in the treatment of a neurological disease, a neurodegenerative disorder, a muscular disease, a neuromuscular disorder, a neuro-oncological disorder, or any disorder associated with the central and/or peripheral nervous systems.

In some embodiments, the viral genome of the AAV particle (e.g., an AAV particle comprising an AAV capsid polypeptide) may comprise a nucleic acid which has been engineered to enable or enhance the expression of an antibody, or antibody binding fragment thereof.

In some embodiments, the encoded antibody of the payload of an AAV particle comprising an AAV capsid variant, described herein comprises at least one immunoglobulin variable domain sequence. An antibody may include, for example, full-length, mature antibodies and antigen-binding fragments of an antibody. For example, an antibody can include a heavy (H) chain variable domain sequence (VH), and a light (L) chain variable domain sequence (VL). In another example, an antibody includes two heavy (H) chain variable domain sequences and two light (L) chain variable domain sequence, thereby forming two antigen binding sites, such as Fab, Fab′, F(ab′)2, Fc, Fd, Fd′, Fv, single chain antibodies (scFv for example), single variable domain antibodies, diabodies (Dab) (bivalent and bispecific), and chimeric (e.g., humanized) antibodies, which may be produced by the modification of whole antibodies or those synthesized de novo using recombinant DNA technologies. These functional antibody fragments, e.g., an antibody binding fragments, retain the ability to selectively bind with their respective antigen or receptor.

In some embodiments, the antibody binding fragment comprises at least one portion of an intact antibody, or recombinant variants thereof, and refers to the antigen binding domain, for example, an antigenic determining variable region of an intact antibody, that is sufficient to confer recognition and specific binding of the antibody fragment to a target, such as an antigen. Examples of antigen binding fragments include: (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a diabody (dAb) fragment, which consists of a VH domain; (vi) a camelid or camelized variable domain; (vii) a single chain Fv (scFv), see e.g., Bird et al. (1988) Science 242:423-426; and Huston et al. (1988) Proc. Natl. Acad. Sci. USA 85:5879-5883); and (viii) a single domain antibody. These antibody fragments are obtained using conventional techniques known to those with skill in the art, and the fragments are screened for utility in the same manner as are intact antibodies. An antibody fragment can also be incorporated into single domain antibodies, maxibodies, minibodies, nanobodies, intrabodies, diabodies, triabodies, tetrabodies, v-NAR and bis-scFv (see, for example, Hollinger and Hudson, Nature Biotechnology 23:1126-1136, 2005).

In some embodiments, the encoded antibody of the payload of an AAV particle described herein comprises a multispecific antibody, e.g., it comprises a plurality of immunoglobulin variable domains sequences, wherein a first immunoglobulin variable domain sequence of the plurality has binding specificity for a first epitope and a second immunoglobulin variable domain sequence of the plurality has binding specificity for a second epitope. In some embodiments, the first and second epitopes are on the same antigen, e.g., the same protein (or subunit of a multimeric protein). In some embodiments, the first and second epitopes overlap. In some embodiments, the first and second epitopes do not overlap. In some embodiments, the first and second epitopes are on different antigens, e.g., the different proteins (or different subunits of a multimeric protein). In some embodiments, a multispecific antibody comprises a third, fourth or fifth immunoglobulin variable domain. In some embodiments, a multispecific antibody is a bispecific antibody, a trispecific antibody, or tetraspecific antibody.

In some embodiments, an encoded multispecific antibody of the payload of an AAV particle described herein is an encoded bispecific antibody. A bispecific antibody has specificity for no more than two antigens. A bispecific antibody is characterized by a first immunoglobulin variable domain sequence which has binding specificity for a first epitope and a second immunoglobulin variable domain sequence that has binding specificity for a second epitope. In some embodiments, the first and second epitopes are on the same antigen, e.g., the same protein (or subunit of a multimeric protein). In some embodiments, the first and second epitopes overlap. In some embodiments, the first and second epitopes do not overlap. In some embodiments, the first and second epitopes are on different antigens, e.g., the different proteins (or different subunits of a multimeric protein).

An antibody or an antibody binding fragment encoded by a viral genome of an AAV particle described herein, may be, but is not limited to, an antibody or antibody fragment that binds to P-amyloid, APOE, tau, SOD1, TDP-43, huntingtin, and/or synuclein. In some embodiments, the encoded payload comprises an antibody or antibody fragment that binds to a neuro-oncology related target, e.g., HER2, EGFR (e.g., EGFRvIII). In some embodiments, the encoded payload comprises an antibody that binds to HER2/neu. In some embodiments, the encoded payload comprises an antibody that binds to P-amyloid. In some embodiments, the encoded payload comprises an antibody that binds to tau.

### Gene Editing System

In some embodiments, the encoded payload of AAV particle comprising an AAV capsid variant described herein comprises a gene editing system or one or more components thereof. In some embodiments, the gene editing system comprises nucleic acid sequences that encode proteins having enzymatic activity to (i) selectively induce double or single stranded breaks in a DNA or RNA sequence, or (ii) substitute, insert or delete a particular base or set of bases of a DNA or RNA sequence in the absence of a double or single stranded break in the DNA or RNA. In some embodiments, the gene editing system includes, but is not limited to a CRISPR-Cas system (including different Cas or Cas-related nucleases), a Zinc finger nuclease, a meganuclease, a TALEN or a base editors. In some embodiments, the gene editing system comprises a chromosomal integration of a transgene, e.g., introduced by a parvovirus vector in the absence of an exogenous nuclease or an enzymatic entity.

### RNAi Agents

In some embodiments, the encoded payload of AAV particle comprising an AAV capsid polypeptide described herein comprises an RNAi agent, e.g., an RNAi agent described herein. In some embodiments, the encoded payload of a viral genome of an AAV particle comprising an AAV capsid variant described herein comprises an RNAi agent, the RNAi, such as but not limited to, a dsRNA, a siRNA, a shRNA, a pre-miRNA, a pri-miRNA, a miRNA, a stRNA, a lncRNA, a piRNA, or a snoRNA. In some embodiments, the encoded payload comprises an RNAi agent for inhibiting expression of a SOD1, MAPT, APOE, HTT, C90RF72, TDP-43, APP, BACE, SNCA, ATXN1, ATXN3, ATXN7, SCN1A-SCN5A, or SCN8A-SCN11A gene, protein, and/or mRNA. In some embodiments, the RNAi agent encoded by a viral genome described herein inhibits SOD1, MAPT, APOE, HTT, C90RF72, TDP-43, APP, BACE, SNCA, ATXN1, ATXN3, ATXN7, SCN1A-SCN5A, or SCN8A-SCN11A.

An AAV particle comprising an AAV capsid variant described herein may comprise a viral genome encoding an RNAi agent, which targets the mRNA of a gene to modulate, e.g., interfere with gene expression and/or protein production.

In some embodiments, the RNAi agent may target a gene at the location of a single-nucleotide polymorphism (SNP) or variant within the nucleotide sequence of the gene.

The RNAi agent may be an siRNA duplex, wherein the siRNA duplex contains an antisense strand (guide strand) and a sense strand (passenger strand) hybridized together forming a duplex structure, wherein the antisense strand is complementary to the nucleic acid sequence of the targeted gene, and wherein the sense strand is homologous to the nucleic acid sequence of the targeted gene. In some aspects, the 5′end of the antisense strand has a 5′ phosphate group and the 3′end of the sense strand contains a 3′hydroxyl group. In other aspects, there are none, one or 2 nucleotide overhangs at the 3′end of each strand.

Each strand of an siRNA duplex targeting a gene of interest may be about 19 to 25, 19 to 24 or 19 to 21 nucleotides in length, preferably about 19 nucleotides, 20 nucleotides, 21 nucleotides, 22 nucleotides, 23 nucleotides, 24 nucleotides, or 25 nucleotides in length.

In one embodiment, an siRNA or dsRNA includes at least two sequences that are complementary to each other. The dsRNA includes a sense strand having a first sequence and an antisense strand having a second sequence. The antisense strand includes a nucleotide sequence that is substantially complementary to at least part of an mRNA encoding the target gene, and the region of complementarity is 30 nucleotides or less, and at least 15 nucleotides in length. Generally, the dsRNA is 19 to 25, 19 to 24 or 19 to 21 nucleotides in length. In some embodiments, the dsRNA is from about 15 to about 25 nucleotides in length, and in other embodiments the dsRNA is from about 25 to about 30 nucleotides in length. In some embodiments, the dsRNA is about 15 nucleotides in length, 16 nucleotides in length, 17 nucleotides in length, 18 nucleotides in length, 19 nucleotides, 20 nucleotides, 21 nucleotides, 22 nucleotides, 23 nucleotides, 24 nucleotides, 25 nucleotides in length, 26 nucleotides in length, 27 nucleotides in length, 28 nucleotides in length, 29 nucleotides in length, or 30 nucleotides in length.

In some embodiments, the encoded RNAi agent is an siRNA.

In some embodiments, the RNAi agent, e.g., an RNAi agent described herein inhibits the expression of the gene, mRNA, and/or protein by at least 10%, at least 20%, at least 25%, at least 30%, at least 35% or at least 40% or more, such as when assayed by a method known in the art. In some embodiments, the RNAi agent inhibits expression of a gene, mRNA, and protein by 50-100%, e.g., by 30%, 40%, 50%, 60%, 70%, 80%, 85%, 90%, 95% and 100%.

In some embodiments, the AAV particle described herein, comprising a viral genome encoding an RNAi agent targeting a gene of interest is administered to a subject in need for treating and/or ameliorating a disease, e.g., a neurological disorder of any disease associated with the central or peripheral nervous systems.

Design of siRNA

An AAV particle described herein (e.g., an AAV particle comprising an AAV capsid polypeptide described herein) may comprise a viral genome encoding a siRNA molecule (e.g., siRNA duplex or encoded dsRNA) that target a gene of interest and suppress target gene expression, mRNA expression, and protein production. In some aspects, the siRNA molecules are designed and used to knock out target gene variants in cells, e.g., transcripts that are identified in neurological disease. In some aspects, the siRNA molecules are designed and used to knock down target gene variants in cells.

Some guidelines for designing siRNAs (for insertion into a viral genome of the AAV particles described herein) have been proposed in the art. These guidelines generally recommend generating a 19-nucleotide duplexed region, symmetric 2-3 nucleotide 3′overhangs, 5-phosphate and 3-hydroxyl groups targeting a region in the gene to be silenced. Other rules that may govern siRNA sequence preference include, but are not limited to, (i) A/U at the 5′ end of the antisense strand; (ii) G/C at the 5′ end of the sense strand; (iii) at least five A/U residues in the 5′ terminal one-third of the antisense strand; and (iv) the absence of any GC stretch of more than 9 nucleotides in length. In accordance with such considerations, together with the specific sequence of a target gene, highly effective siRNA molecules essential for suppressing mammalian target gene expression may be readily designed.

In one embodiment, the sense and/or antisense strand is designed based on the method and rules outlined in European Patent Publication No. EP1752536, the contents of which are herein incorporated by reference in their entirety. As a non-limiting example, the 3′-terminal base of the sequence is adenine, thymine or uracil. As a non-limiting example, the 5′-terminal base of the sequence is guanine or cytosine. As a non-limiting example, the 3′-terminal sequence comprises seven bases rich in one or more bases of adenine, thymine and uracil.

In one embodiment, an siRNA molecule comprises a sense strand and a complementary antisense strand in which both strands are hybridized together to form a duplex structure. The antisense strand has sufficient complementarity to the target mRNA sequence to direct target-specific RNAi, e.g., the siRNA molecule has a sequence sufficient to trigger the destruction of the target mRNA by the RNAi machinery or process.

In some embodiments, the antisense strand and target mRNA sequences have 100% complementarity. The antisense strand may be complementary to any part of the target mRNA sequence. Neither the identity of the sense sequence nor the homology of the antisense sequence need be 100% complementary to the target.

In other embodiments, the antisense strand and target mRNA sequences comprise at least one mismatch. As a non-limiting example, the antisense strand and the target mRNA sequence have at least 50-90%, 50-95%, 50-99%, 60-70%, 60-80%, 60-90%, 60-95%, 60-99%, 70-80%, 70-90%, 70-95%, 70- 99%, 80-90%, 80-95%, 80-99%, 90-95%, 90-99% or 95-99% complementary.

The siRNA molecule may have a length from about 10-50 or more nucleotides, e.g., each strand comprising 10-50 nucleotides (or nucleotide analogs). Preferably, the siRNA molecule has a length from about 15-30, e.g., 15, 16, 17, 18, 19, 20, 21, 22, 23, 24, 25, 26, 27, 28, 29, or 30 nucleotides in each strand, wherein one of the strands is sufficiently complementary to a target region. In one embodiment, the siRNA molecule has a length from about 19 to 25, 19 to 24 or 19 to 21 nucleotides.

In some embodiments, the siRNA molecule can be a synthetic RNA duplex comprising about 19 nucleotides to about 25 nucleotides, and two overhanging nucleotides at the 3-end.

The siRNA molecule may comprise an antisense sequence and a sense sequence, or a fragment or variant thereof. As a non-limiting example, the antisense sequence and the sense sequence have at least 50-90%, 50-95%, 50-99%, 60-70%, 60-80%, 60-90%, 60-95%, 60-99%, 70-80%, 70-90%, 70-95%, 70-99%, 80-90%, 80-95%, 80-99%, 90-95%, 90-99% or 95-99% complementary.

The sense and antisense sequences may be completely complementary across a substantial portion of their length. In other embodiments, the sense sequence and antisense sequence may be at least 70, 80, 90, 95 or 99% complementary across independently at least 50, 60, 70, 80, 85, 90, 95, or 99% of the length of the strands.

In some embodiments, the sense and antisense strands of a siRNA duplex are linked by a short spacer sequence leading to the expression of a stem-loop structure termed short hairpin RNA (shRNA). The hairpin is recognized and cleaved by Dicer, thus generating mature siRNA molecules.

In some embodiments, the siRNA molecules, as well as associated spacer and/or flanking regions once designed, can be encoded by the viral genome of the AAV particles described herein, for delivery to a cell.

### Molecular Scaffold

In some embodiments, the siRNA molecules may be encoded in a modulatory polynucleotide which also comprises a molecular scaffold.

In some embodiments, the modulatory polynucleotide which comprises the payload (e.g., siRNA, miRNA or other RNAi agent described herein) includes a molecular scaffold which comprises a 5′ flanking sequence, a loop region, and/or a 3′ flanking region. In some embodiments a 5′ or 3′ flanking region may be of any length and may a wild type microRNA sequence or a portion thereof, or may be completely artificial. A 3′ flanking sequence may mirror the 5′ flanking sequence in size and origin. Either flanking sequence may be absent. In one embodiment, both the 5′ and 3′ flanking sequences are absent. The 3′ flanking sequence may optionally contain one or more CNNC motifs, where “N” represents any nucleotide. In some embodiments, the loop comprises at least one UGUG motif. In some embodiments, the UGUG motif is located at the 5′ terminus of the loop. In some embodiments the 5′ and 3′ flanking sequences are the same sequence. In some embodiments they differ by 2%, 3%, 4%, 5%, 10%, 20% or more than 30% when aligned to each other.

In some embodiments, modulatory polynucleotide comprises a stem loop structure. In some embodiments, the modulatory polynucleotide comprises in 5′ to 3′ order: a 5′ flanking sequence, a guide strand sequence, a loop region, a passenger strand sequence, and a 3′ flanking sequence. In some embodiments, the modulatory polynucleotide comprises in 5′ to 3′ order: a 5′ flanking sequence, a passenger strand sequence, a loop region, a guide strand sequence, and a 3′ flanking sequence.

In one embodiment, the molecular scaffold comprises a dual-function targeting modulatory polynucleotide.

In one embodiment, the molecular scaffold may comprise one or more linkers known in the art. The linkers may separate regions or one molecular scaffold from another. As a non-limiting example, the molecular scaffold may be polycistronic.

In one embodiment, the modulatory polynucleotide is designed using at least one of the following properties: loop variant, seed mismatch/bulge/wobble variant, stem mismatch, loop variant and basal stem mismatch variant, seed mismatch and basal stem mismatch variant, stem mismatch and basal stem mismatch variant, seed wobble and basal stem wobble variant, or a stem sequence variant.

### AAV Production

Viral production disclosed herein describes processes and methods for producing AAV particles (with enhanced, improved and/or increased tropism for a target tissue).

In some embodiments, disclosed herein is a method of making AAV particle of the present disclosure, e.g., an AAV particle comprising an AAV capsid polypeptide the method comprising: (i) providing a host cell comprising a baculovirus expression construct described herein comprising a viral genome described herein and (ii) incubating the host cell under conditions suitable to enclose the viral genome in an AAV capsid polypeptide, thereby making the AAV particle. In some embodiments, the method comprises prior to step (i), introducing a first baculovirus expression construct comprising the viral genome into a cell. In some embodiments, the host cell comprises a second baculovirus expression construct described herein encoding the AAV capsid polypeptide. In some embodiments, the second baculovirus expression construct is introduced into the host cell prior to, concurrently with, or after the first baculovirus expression construct. In some embodiments, the AAV particle described herein is an isolated AAV particle. In some embodiments, the AAV particle described herein is a recombinant AAV particle.

Any method known in the art may be used for the preparation of AAV particles. In some embodiments, AAV particles are produced in mammalian cells (e.g., HEK293). In another embodiment, AAV particles are produced in insect cells (e.g., Sf9).

Methods of making AAV particles are well known in the art and are described in, e.g., U.S. Pat. Nos. 6,204,059, 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508, 5,064,764, 6,194,191, 6,566,118, 8,137,948; or International Publication Nos. WO1996039530, WO1998010088, WO1999014354, WO1999015685, WO1999047691, WO2000055342, WO2000075353 and WO2001023597; Methods In Molecular Biology, ed. Richard, Humana Press, NJ (1995); O'Reilly et al., Baculovirus Expression Vectors, A Laboratory Manual, Oxford Univ. Press (1994); Samulski et al., J. Vir. 63:3822-8 (1989); Kajigaya et al., Proc. Nat'l. Acad. Sci. USA 88: 4646-50 (1991); Ruffing et al., J. Vir. 66:6922-30 (1992); Kimbauer et al., Vir., 219:37-44 (1996); Zhao et al., Vir. 272:382-93 (2000); the contents of each of which are herein incorporated by reference in their entirety. In some embodiments, the AAV particles are made using the methods described in International Patent Publication WO2015191508, the contents of which are herein incorporated by reference in their entirety.

### Viral Expression Constructs

The AAV production system described herein comprises one or more baculovirus expression constructs comprising AAV expression constructs which can be transfected/transduced into a viral production cell (e.g., Sf9). In certain embodiments, a baculovirus expression construct (e.g., BAC) described herein comprises an AAV expression construct and/or a payload expression construct. In certain embodiments, a viral expression construct of the present disclosure can be a baculovirus expression vector (BEV). In certain embodiments, a viral expression construct of the present disclosure can be a BIIC which includes a BEV. In some embodiments, expressionBac refers to a baculovirus expression construct described herein which comprises a viral expression construct (e.g., an AAV expression construct) and/or viral expression region. Viral production cells (e.g., Sf9 cells) may be transfected with expressionBacs and/or with BIICs comprising expressionBacs. In some embodiments, the baculovirus expression construct or variant baculovirus genome comprises an AAV expression construct as described, e.g., in PCT/US2022/018687, the content of which are hereby incorporated by reference in their entirety. In some embodiments, the baculovirus expression construct or variant baculovirus genome comprises an AAV expression construct as described, e.g., in PCT/US2022/018787, the content of which are hereby incorporated by reference in their entirety. Baculovirus expression constructs can be generated using similar protocols as described in Examples 1-8 of PCT/US2022/018687, the content of which are hereby incorporated by reference in their entirety. Baculovirus expression constructs can be generated using similar protocols as described in Examples 1-5 of PCT/US2022/018787, the content of which are hereby incorporated by reference in their entirety.

In some embodiments, one, two, three, four, five, six, or more AAV expression constructs can be employed to produce AAV particles in viral production cells. For instance, five AAV expression constructs may individually encode AAV VP1, AAV VP2, AAV VP3, Rep52, Rep78, and with an accompanying payload construct comprising a payload polynucleotide and at least one AAV ITR. In another embodiment, the AAV expression constructs described herein may be used to express, for example, Rep52 and Rep40, or Rep78 and Rep 68, or Rep78 and Rep52. Expression constructs may comprise any combination of VP1, VP2, VP3, Rep52/Rep40, Rep78/Rep52, and Rep78/Rep68 coding sequences.

In some embodiments, a baculovirus expression construct or a variant baculovirus genome described herein comprises an AAV expression construct comprising a VP-coding region. In some embodiments, an AAV expression construct can comprise a VP-coding region; a VP-coding region is a nucleotide sequence which comprises a VP nucleotide sequence encoding VP1, VP2, VP3, or a combination thereof. In some embodiments, a VP-coding region can be codon optimized, e.g., for a mammalian cell or an insect cell. In some embodiments, a VP-coding region or nucleotide sequence can be codon optimized for an insect cell, e.g., a Spodoptera frugiperda cell, such as Sf9 or Sf21. In some embodiments, the viral expression construct comprises a first VP-coding region which comprises a nucleotide sequence encoding one or more AAV capsid proteins selected from VP1, VP2, and VP3; and a second VP-coding region which comprises a nucleotide sequence encoding one or more AAV capsid proteins selected from VP1, VP2, and VP3.

In some embodiments, the first VP-coding region encodes AAV capsid proteins of an AAV serotype, e.g., AAV2, AAV9 or AAVPHPN. In some embodiments, the second VP-coding region encodes AAV capsid proteins of an AAV serotype, e.g., AAV2, AAV9 or AAVPHPN. In some embodiments, the AAV serotype of the first VP-coding region is the same as the AAV serotype of the second VP-coding region. In some embodiments, the AAV serotype of the first VP-coding region is different from the AAV serotype of the second VP-coding region.

Structural VP proteins, VP1, VP2, and VP3 of a viral expression construct can be encoded in a single open reading frame regulated by utilization of both alternative splice acceptor and non-canonical translational initiation codons. VP1, VP2, and VP3 can be transcribed and translated from a single transcript in which both in-frame and/or out-of-frame start codons are engineered to control the VP1:VP2:VP3 ratio produced by the nucleotide transcript.

In some embodiments, the VP-coding region comprises a nucleotide sequence encoding: (i) primarily a VP1 protein, e.g., at least 50%, 60%, 70%, 80%, 90% or more VP1 protein relative to a VP2 protein and/or a VP3 protein; (ii) a VP1 protein only; (iii) a VP1 protein, but not a VP2 protein or a VP3 protein; (iv) primarily a VP2 protein, e.g., at least about 50%, 60%, 70%, 80%, 90% or more VP2 protein relative to a VP1 protein and/or a VP3 protein; (v) a VP2 protein only; (vi) a VP2 protein, but not a VP1 protein or a VP3 protein; (vii) a VP3 protein only; (viii) a VP3 protein, but not a VP1 protein or a VP2 protein; (ix) a VP1 protein and a VP2 protein, but not a VP3 protein; (x) a VP1 protein and a VP3 protein, but not a VP2 protein; (xi) a VP2 protein and a VP3 protein, but not a VP1 protein; (xii) a VP1 protein, a VP2 protein, and a VP3 protein.

In some embodiments, the VP-coding region comprises a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein. In some embodiments, the VP-coding region comprises a single polycistronic ORF encoding a VP1 protein, a VP2 protein, and a VP3 protein. In some embodiments, the ORF encoding the VP1 protein comprises an ACG start codon, the ORF encoding the VP2 protein comprises an ACG start codon, and the ORF encoding the VP3 protein comprises an ATG start codon. In some embodiments, the ORF encoding the VP1 protein comprises an ATG start codon, the ORF encoding the VP2 protein comprises an ACG start codon, and the ORF encoding the VP3 protein comprises an ATG start codon.

In some embodiments, the VP-coding region encodes an AAV1 capsid protein, an AAV2 capsid protein, an AAV3 capsid protein, an AAV4 capsid protein, an AAV5 capsid protein, an AAV6 capsid protein, an AAV8 capsid protein, an AAV9 capsid protein, an AAVrh10 capsid protein or a variant of any of the aforesaid capsid proteins. In some embodiments, the VP-coding region encodes an AAV5 capsid protein or variant thereof, or an AAV9 capsid protein or variant thereof. In some embodiments, the VP-coding region encodes a capsid protein as provided in WO2021230987, WO2019028306, WO2019222329, WO2020077165, WO2020028751, WO2020223280, WO2019222444, WO2019222441, PCT/US2022/018687, or WO2017100671, the contents of which are hereby incorporated by reference in their entirety. In some embodiments, the VP-coding region encodes a capsid protein encoded by or comprising a sequence as provided in Table 2, or a sequence substantially identical (e.g., having at least about 70%, 75%, 80%, 85%, 90%, 92%, 95%, 97%, 98%, or 99% sequence identity) to any of the aforesaid sequences.

In some embodiments, the VP-coding encodes a VP1 protein comprising the amino acid sequence of any of SEQ ID NOs: 46, 47, 71, or 168, or an amino acid sequence at least 70%, 75%, 80%, 85%, 90%, 95%, 96%, 97%, 98%, or 99% identical to any of the aforesaid amino acid sequences. In some embodiments, the VP-coding region comprises the nucleotide sequence of any of SEQ ID NOs: 43-45, 72, 169, 205, 212, or 213, or a nucleotide sequence at least 70%, 75%, 80%, 85%, 90%, 95%, 96%, 97%, 98%, or 99% identical to any of the aforesaid nucleotide sequences.

In some embodiments, the VP-coding region encodes a VP2 protein e.g., a fragment or a portion, of any of SEQ ID NOs: 46, 47, 71, or 168, or an amino acid sequence at least 70%, 75%, 80%, 85%, 90%, 95%, 96%, 97%, 98%, or 99% identical to any of the aforesaid amino acid sequences. In some embodiments, the VP2 protein comprises amino acids 138-736 or SEQ ID NOs: 46, 47, or 71; or amino acids 137-724 of SEQ ID NO: 168. In some embodiments, the VP-coding region comprises a nucleotide sequence encoding a VP2 protein e.g., a fragment or a portion, of any of SEQ ID NOs: 43-45, 72, 169, 205, 212, or 213, or a nucleotide sequence at least 70%, 75%, 80%, 85%, 90%, 95%, 96%, 97%, 98%, or 99% identical to any of the aforesaid nucleotide sequences. In some embodiments, the nucleotide sequence encoding the VP2 protein comprises nucleotides 412-2211 of SEQ ID NOs: 43-45, 72, 205, or 212; or nucleotides 409-2175 of SEQ ID NO: 169 or 213. In some embodiments, the nucleotide sequence encoding the VP2 protein comprises nucleotides 418-2211 of SEQ ID NOs: 44 and 45.

In some embodiments, the VP-coding region encodes a VP3 protein e.g., a fragment or a portion, of any of SEQ ID NOs: 46, 47, 71, or 168, or an amino acid sequence at least 70%, 75%, 80%, 85%, 90%, 95%, 96%, 97%, 98%, or 99% identical to any of the aforesaid amino acid sequences. In some embodiments, the VP3 protein comprises amino acids 203-736 of SEQ ID NOs: 46, 47, or 71; or amino acids 193-724 of SEQ ID NO: 168. In some embodiments, the VP-coding region comprises a nucleotide sequence encoding a VP3 protein e.g., a fragment or a portion, of any of SEQ ID NOs: 43-45, 72, 169, 205, 212, or 213, or a nucleotide sequence at least 70%, 75%, 80%, 85%, 90%, 95%, 96%, 97%, 98%, or 99% identical to any of the aforesaid nucleotide sequences. In some embodiments, the nucleotide sequence encoding the VP3 protein comprises nucleotides 607-2211 of SEQ ID NOs: 43-45, 72, 205, or 212; or nucleotides 577-2175 of SEQ ID NO: 169 or 213. In some embodiments, the nucleotide sequence encoding the VP3 protein comprises nucleotides 613-2211 of SEQ ID NOs: 44 and 45.

In some embodiments, the nucleotide sequence of the VP-coding region is codon optimized for an insect cell, optionally a Spodopterafrugiperda insect cell (e.g., an Sf9 insect cell).

TABLE 2
Exemplary full length capsid sequences

| | SEQ | |
|---|---|---|
| | ID | |
| Description | NO: | Sequence |
| AAV9 | 43 | ATGGCTGCCGATGGTTATCTTCCAGATTGGCTCGAGGACAACCTTAGTGAAGGAATTCG |
| (DNA v1) | | CGAGTGGTGGGCTTTGAAACCTGGAGCCCCTCAACCCAAGGCAAATCAACAACATCAAG |
| | | ACAACGCTCGAGGTCTTGTGCTTCCGGGTTACAAATACCTTGGACCCGGCAACGGACTC |
| | | GACAAGGGGGAGCCGGTCAACGCAGCAGACGCGGCGGCCCTCGAGCACGACAAGGCCTA |
| | | CGACCAGCAGCTCAAGGCCGGAGACAACCCGTACCTCAAGTACAACCACGCCGACGCCG |
| | | AGTTCCAGGAGCGGCTCAAAGAAGATACGTCTTTTGGGGGCAACCTCGGGCGAGCAGTC |
| | | TTCCAGGCCAAAAAGAGGCTTCTTGAACCTCTTGGTCTGGTTGAGGAAGCGGCTAAGAC |
| | | GGCTCCTGGAAAGAAGAGGCCTGTAGAGCAGTCTCCTCAGGAACCGGACTCCTCCGCGG |
| | | GTATTGGCAAATCGGGTGCACAGCCCGCTAAAAAGAGACTCAATTTCGGTCAGACTGGC |
| | | GACACAGAGTCAGTCCCAGACCCTCAACCAATCGGAGAACCTCCCGCAGCCCCCTCAGG |
| | | TGTGGGATCTCTTACAATGGCTTCAGGTGGTGGCGCACCAGTGGCAGACAATAACGAAG |
| | | GTGCCGATGGAGTGGGTAGTTCCTCGGGAAATTGGCATTGCGATTCCCAATGGCTGGGG |
| | | GACAGAGTCATCACCACCAGCACCCGAACCTGGGCCCTGCCCACCTACAACAATCACCT |
| | | CTACAAGCAAATCTCCAACAGCACATCTGGAGGATCTTCAAATGACAACGCCTACTTCG |
| | | GCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCTAAGCGACTCAACTT |
| | | CAAGCTCTTCAACATTCAGGTCAAAGAGGTTACGGACAACAATGGAGTCAAGACCATCG |
| | | CCAATAACCTTACCAGCACGGTCCAGGTCTTCACGGACTCAGACTATCAGCTCCCGTAC |
| | | GTGCTCGGGTCGGCTCACGAGGGCTGCCTCCCGCCGTTCCCAGCGGACGTTTTCATGAT |
| | | TCCTCAGTACGGGTATCTGACGCTTAATGATGGAAGCCAGGCCGTGGGTCGTTCGTCCT |
| | | TTTACTGCCTGGAATATTTCCCGTCGCAAATGCTAAGAACGGGTAACAACTTCCAGTTC |
| | | AGCTACGAGTTTGAGAACGTACCTTTCCATAGCAGCTACGCTCACAGCCAAAGCCTGGA |
| | | CCGACTAATGAATCCACTCATCGACCAATACTTGTACTATCTCTCAAAGACTATTAACG |
| | | GTTCTGGACAGAATCAACAAACGCTAAAATTCAGTGTGGCCGGACCCAGCAACATGGCT |
| | | GTCCAGGGAAGAAACTACATACCTGGACCCAGCTACCGACAACAACGTGTCTCAACCAC |
| | | TGTGACTCAAAACAACAACAGCGAATTTGCTTGGCCTGGAGCTTCTTCTTGGGCTCTCA |
| | | ATGGACGTAATAGCTTGATGAATCCTGGACCTGCTATGGCCAGCCACAAAGAAGGAGAG |
| | | GACCGTTTCTTTCCTTTGTCTGGATCTTTAATTTTTGGCAAACAAGGAACTGGAAGAGA |
| | | CAACGTGGATGCGGACAAAGTCATGATAACCAACGAAGAAGAAATTAAAACTACTAACC |
| | | CGGTAGCAACGGAGTCCTATGGACAAGTGGCCACAAACCACCAGAGTGCCCAAGCACAG |
| | | GCGCAGACCGGCTGGGTTCAAAACCAAGGAATACTTCCGGGTATGGTTTGGCAGGACAG |
| | | AGATGTGTACCTGCAAGGACCCATTTGGGCCAAAATTCCTCACACGGACGGCAACTTTC |
| | | ACCCTTCTCCGCTGATGGGAGGGTTTGGAATGAAGCACCCGCCTCCTCAGATCCTCATC |
| | | AAAAACACACCTGTACCTGCGGATCCTCCAACGGCCTTCAACAAGGACAAGCTGAACTC |
| | | TTTCATCACCCAGTATTCTACTGGCCAAGTCAGCGTGGAGATCGAGTGGGAGCTGCAGA |
| | | AGGAAAACAGCAAGCGCTGGAACCCGGAGATCCAGTACACTTCCAACTATTACAAGTCT |
| | | AATAATGTTGAATTTGCTGTTAATACTGAAGGTGTATATAGTGAACCCCGCCCCATTGG |
| | | CACCAGATACCTGACTCGTAATCTGTAA |
| AAV9 | 205 | ACGGCTGCCGACGGTTATCTACCCGATTGGCTCGAGGACAACCTTAGTGAAGGAATTCG |
| (DNA v2) | | CGAGTGGTGGGCTTTGAAACCTGGAGCCCCTCAACCCAAGGCAAATCAACAACATCAAG |
| | | ACAACGCTCGAGGTCTTGTGCTTCCGGGTTACAAATACCTTGGACCCGGCAACGGACTC |
| | | GACAAGGGGGAGCCGGTCAACGCAGCAGACGCGGCGGCCCTCGAGCACGACAAGGCCTA |
| | | CGACCAGCAGCTCAAGGCCGGAGACAACCCGTACCTCAAGTACAACCACGCCGACGCCG |
| | | AGTTCCAGGAGCGGCTCAAAGAAGATACGTCTTTTGGGGGCAACCTCGGGCGAGCAGTC |
| | | TTCCAGGCCAAAAAGAGGCTTCTTGAACCTCTTGGTCTGGTTGAGGAAGCGGCTAAGAC |
| | | GGCTCCTGGAAAGAAGAGGCCTGTAGAGCAGTCTCCTCAGGAACCGGACTCCTCCGCGG |
| | | GTATTGGCAAATCGGGTGCACAGCCCGCTAAAAAGAGACTCAATTTCGGTCAGACTGGC |
| | | GACACAGAGTCAGTCCCAGACCCTCAACCAATCGGAGAACCTCCCGCAGCCCCCTCAGG |
| | | TGTGGGATCTCTTACAATGGCTTCAGGTGGTGGCGCACCAGTGGCAGACAATAACGAAG |
| | | GTGCCGATGGAGTGGGTAGTTCCTCGGGAAATTGGCATTGCGATTCCCAATGGCTGGGG |
| | | GACAGAGTCATCACCACCAGCACCCGAACCTGGGCCCTGCCCACCTACAACAATCACCT |
| | | CTACAAGCAAATCTCCAACAGCACATCTGGAGGATCTTCAAATGACAACGCCTACTTCG |
| | | GCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCTAAGCGACTCAACTT |
| | | CAAGCTCTTCAACATTCAGGTCAAAGAGGTTACGGACAACAATGGAGTCAAGACCATCG |
| | | CCAATAACCTTACCAGCACGGTCCAGGTCTTCACGGACTCAGACTATCAGCTCCCGTAC |
| | | GTGCTCGGGTCGGCTCACGAGGGCTGCCTCCCGCCGTTCCCAGCGGACGTTTTCATGAT |
| | | TCCTCAGTACGGGTATCTGACGCTTAATGATGGAAGCCAGGCCGTGGGTCGTTCGTCCT |
| | | TTTACTGCCTGGAATATTTCCCGTCGCAAATGCTAAGAACGGGTAACAACTTCCAGTTC |
| | | AGCTACGAGTTTGAGAACGTACCTTTCCATAGCAGCTACGCTCACAGCCAAAGCCTGGA |
| | | CCGACTAATGAATCCACTCATCGACCAATACTTGTACTATCTCTCAAAGACTATTAACG |
| | | GTTCTGGACAGAATCAACAAACGCTAAAATTCAGTGTGGCCGGACCCAGCAACATGGCT |
| | | GTCCAGGGAAGAAACTACATACCTGGACCCAGCTACCGACAACAACGTGTCTCAACCAC |
| | | TGTGACTCAAAACAACAACAGCGAATTTGCTTGGCCTGGAGCTTCTTCTTGGGCTCTCA |
| | | ATGGACGTAATAGCTTGATGAATCCTGGACCTGCTATGGCCAGCCACAAAGAAGGAGAG |
| | | GACCGTTTCTTTCCTTTGTCTGGATCTTTAATTTTTGGCAAACAAGGAACTGGAAGAGA |
| | | CAACGTGGATGCGGACAAAGTCATGATAACCAACGAAGAAGAAATTAAAACTACTAACC |
| | | CGGTAGCAACGGAGTCCTATGGACAAGTGGCCACAAACCACCAGAGTGCCCAAGCACAG |
| | | GCGCAGACCGGCTGGGTTCAAAACCAAGGAATACTTCCGGGTATGGTTTGGCAGGACAG |
| | | AGATGTGTACCTGCAAGGACCCATTTGGGCCAAAATTCCTCACACGGACGGCAACTTTC |
| | | ACCCTTCTCCGCTGATGGGAGGGTTTGGAATGAAGCACCCGCCTCCTCAGATCCTCATC |
| | | AAAAACACACCTGTACCTGCGGATCCTCCAACGGCCTTCAACAAGGACAAGCTGAACTC |
| | | TTTCATCACCCAGTATTCTACTGGCCAAGTCAGCGTGGAGATCGAGTGGGAGCTGCAGA |
| | | AGGAAAACAGCAAGCGCTGGAACCCGGAGATCCAGTACACTTCCAACTATTACAAGTCT |
| | | AATAATGTTGAATTTGCTGTTAATACTGAAGGTGTATATAGTGAACCCCGCCCCATTGG |
| | | CACCAGATACCTGACTCGTAATCTGTAA |
| AAV9 | 44 | cggtgtatggcTGCCGACGGTTATCTACCCGATTGGCTCGAGGACAACCTTAGTGAAGG |
| (DNA with | | AATTCGCGAGTGGTGGGCTTTGAAACCTGGAGCCCCTCAACCCAAGGCAAATCAACAAC |
| VP1aug13 | | ATCAAGACAACGCTCGAGGTCTTGTGCTTCCGGGTTACAAATACCTTGGACCCGGCAAC |
| modified | | GGACTCGACAAGGGGGAGCCGGTCAACGCAGCAGACGCGGCGGCCCTCGAGCACGACAA |
| Kozak | | GGCCTACGACCAGCAGCTCAAGGCCGGAGACAACCCGTACCTCAAGTACAACCACGCCG |
| underlined) | | ACGCCGAGTTCCAGGAGCGGCTCAAAGAAGATACGTCTTTTGGGGGCAACCTCGGGCGA |
| | | GCAGTCTTCCAGGCCAAAAAGAGGCTTCTTGAACCTCTTGGTCTGGTTGAGGAAGCGGC |
| | | TAAGACGGCTCCTGGAAAGAAGAGGCCTGTAGAGCAGTCTCCTCAGGAACCGGACTCCT |
| | | CCGCGGGTATTGGCAAATCGGGTGCACAGCCCGCTAAAAAGAGACTCAATTTCGGTCAG |
| | | ACTGGCGACACAGAGTCAGTCCCAGACCCTCAACCAATCGGAGAACCTCCCGCAGCCCC |
| | | CTCAGGTGTGGGATCTCTTACAATGGCTTCAGGTGGTGGCGCACCAGTGGCAGACAATA |
| | | ACGAAGGTGCCGATGGAGTGGGTAGTTCCTCGGGAAATTGGCATTGCGATTCCCAATGG |
| | | CTGGGGGACAGAGTCATCACCACCAGCACCCGAACCTGGGCCCTGCCCACCTACAACAA |
| | | TCACCTCTACAAGCAAATCTCCAACAGCACATCTGGAGGATCTTCAAATGACAACGCCT |
| | | ACTTCGGCTACAGCACCCCCTGGGGGTATTTTGACTTCAACAGATTCCACTGCCACTTC |
| | | TCACCACGTGACTGGCAGCGACTCATCAACAACAACTGGGGATTCCGGCCTAAGCGACT |
| | | CAACTTCAAGCTCTTCAACATTCAGGTCAAAGAGGTTACGGACAACAATGGAGTCAAGA |
| | | CCATCGCCAATAACCTTACCAGCACGGTCCAGGTCTTCACGGACTCAGACTATCAGCTC |
| | | CCGTACGTGCTCGGGTCGGCTCACGAGGGCTGCCTCCCGCCGTTCCCAGCGGACGTTTT |
| | | CATGATTCCTCAGTACGGGTATCTGACGCTTAATGATGGAAGCCAGGCCGTGGGTCGTT |
| | | CGTCCTTTTACTGCCTGGAATATTTCCCGTCGCAAATGCTAAGAACGGGTAACAACTTC |
| | | CAGTTCAGCTACGAGTTTGAGAACGTACCTTTCCATAGCAGCTACGCTCACAGCCAAAG |
| | | CCTGGACCGACTAATGAATCCACTCATCGACCAATACTTGTACTATCTCTCAAAGACTA |
| | | TTAACGGTTCTGGACAGAATCAACAAACGCTAAAATTCAGTGTGGCCGGACCCAGCAAC |
| | | ATGGCTGTCCAGGGAAGAAACTACATACCTGGACCCAGCTACCGACAACAACGTGTCTC |
| | | AACCACTGTGACTCAAAACAACAACAGCGAATTTGCTTGGCCTGGAGCTTCTTCTTGGG |
| | | CTCTCAATGGACGTAATAGCTTGATGAATCCTGGACCTGCTATGGCCAGCCACAAAGAA |
| | | GGAGAGGACCGTTTCTTTCCTTTGTCTGGATCTTTAATTTTTGGCAAACAAGGAACTGG |
| | | AAGAGACAACGTGGATGCGGACAAAGTCATGATAACCAACGAAGAAGAAATTAAAACTA |
| | | CTAACCCGGTAGCAACGGAGTCCTATGGACAAGTGGCCACAAACCACCAGAGTGCCCAA |
| | | GCACAGGCGCAGACCGGCTGGGTTCAAAACCAAGGAATACTTCCGGGTATGGTTTGGCA |
| | | GGACAGAGATGTGTACCTGCAAGGACCCATTTGGGCCAAAATTCCTCACACGGACGGCA |
| | | ACTTTCACCCTTCTCCGCTGATGGGAGGGTTTGGAATGAAGCACCCGCCTCCTCAGATC |
| | | CTCATCAAAAACACACCTGTACCTGCGGATCCTCCAACGGCCTTCAACAAGGACAAGCT |
| | | GAACTCTTTCATCACCCAGTATTCTACTGGCCAAGTCAGCGTGGAGATCGAGTGGGAGC |
| | | TGCAGAAGGAAAACAGCAAGCGCTGGAACCCGGAGATCCAGTACACTTCCAACTATTAC |
| | | AAGTCTAATAATGTTGAATTTGCTGTTAATACTGAAGGTGTATATAGTGAACCCCGCCC |
| | | CATTGGCACCAGATACCTGACTCGTAATCTGTAA |
| AAV9 | 45 | cggtgtatgagTGCCGACGGTTATCTACCCGATTGGCTCGAGGACAACCTTAGTGAAGG |
| (DNA with | | AATTCGCGAGTGGTGGGCTTTGAAACCTGGAGCCCCTCAACCCAAGGCAAATCAACAAC |
| VP1aug14 | | ATCAAGACAACGCTCGAGGTCTTGTGCTTCCGGGTTACAAATACCTTGGACCCGGCAAC |
| modified | | GGACTCGACAAGGGGGAGCCGGTCAACGCAGCAGACGCGGCGGCCCTCGAGCACGACAA |
| Kozak | | GGCCTACGACCAGCAGCTCAAGGCCGGAGACAACCCGTACCTCAAGTACAACCACGCCG |
| underlined) | | ACGCCGAGTTCCAGGAGCGGCTCAAAGAAGATACGTCTTTTGGGGGCAACCTCGGGCGA |
| | | GCAGTCTTCCAGGCCAAAAAGAGGCTTCTTGAACCTCTTGGTCTGGTTGAGGAAGCGGC |
| | | TAAGACGGCTCCTGGAAAGAAGAGGCCTGTAGAGCAGTCTCCTCAGGAACCGGACTCCT |
| | | CCGCGGGTATTGGCAAATCGGGTGCACAGCCCGCTAAAAAGAGACTCAATTTCGGTCAG |
| | | ACTGGCGACACAGAGTCAGTCCCAGACCCTCAACCAATCGGAGAACCTCCCGCAGCCCC |
| | | CTCAGGTGTGGGATCTCTTACAATGGCTTCAGGTGGTGGCGCACCAGTGGCAGACAATA |
| | | ACGAAGGTGCCGATGGAGTGGGTAGTTCCTCGGGAAATTGGCATTGCGATTCCCAATGG |
| | | CTGGGGGACAGAGTCATCACCACCAGCACCCGAACCTGGGCCCTGCCCACCTACAACAA |
| | | TCACCTCTACAAGCAAATCTCCAACAGCACATCTGGAGGATCTTCAAATGACAACGCCT |
| | | ACTTCGGCTACAGCACCCCCTGGGGGTATTTTGACTTCAACAGATTCCACTGCCACTTC |
| | | TCACCACGTGACTGGCAGCGACTCATCAACAACAACTGGGGATTCCGGCCTAAGCGACT |
| | | CAACTTCAAGCTCTTCAACATTCAGGTCAAAGAGGTTACGGACAACAATGGAGTCAAGA |
| | | CCATCGCCAATAACCTTACCAGCACGGTCCAGGTCTTCACGGACTCAGACTATCAGCTC |
| | | CCGTACGTGCTCGGGTCGGCTCACGAGGGCTGCCTCCCGCCGTTCCCAGCGGACGTTTT |
| | | CATGATTCCTCAGTACGGGTATCTGACGCTTAATGATGGAAGCCAGGCCGTGGGTCGTT |
| | | CGTCCTTTTACTGCCTGGAATATTTCCCGTCGCAAATGCTAAGAACGGGTAACAACTTC |
| | | CAGTTCAGCTACGAGTTTGAGAACGTACCTTTCCATAGCAGCTACGCTCACAGCCAAAG |
| | | CCTGGACCGACTAATGAATCCACTCATCGACCAATACTTGTACTATCTCTCAAAGACTA |
| | | TTAACGGTTCTGGACAGAATCAACAAACGCTAAAATTCAGTGTGGCCGGACCCAGCAAC |
| | | ATGGCTGTCCAGGGAAGAAACTACATACCTGGACCCAGCTACCGACAACAACGTGTCTC |
| | | AACCACTGTGACTCAAAACAACAACAGCGAATTTGCTTGGCCTGGAGCTTCTTCTTGGG |
| | | CTCTCAATGGACGTAATAGCTTGATGAATCCTGGACCTGCTATGGCCAGCCACAAAGAA |
| | | GGAGAGGACCGTTTCTTTCCTTTGTCTGGATCTTTAATTTTTGGCAAACAAGGAACTGG |
| | | AAGAGACAACGTGGATGCGGACAAAGTCATGATAACCAACGAAGAAGAAATTAAAACTA |
| | | CTAACCCGGTAGCAACGGAGTCCTATGGACAAGTGGCCACAAACCACCAGAGTGCCCAA |
| | | GCACAGGCGCAGACCGGCTGGGTTCAAAACCAAGGAATACTTCCGGGTATGGTTTGGCA |
| | | GGACAGAGATGTGTACCTGCAAGGACCCATTTGGGCCAAAATTCCTCACACGGACGGCA |
| | | ACTTTCACCCTTCTCCGCTGATGGGAGGGTTTGGAATGAAGCACCCGCCTCCTCAGATC |
| | | CTCATCAAAAACACACCTGTACCTGCGGATCCTCCAACGGCCTTCAACAAGGACAAGCT |
| | | GAACTCTTTCATCACCCAGTATTCTACTGGCCAAGTCAGCGTGGAGATCGAGTGGGAGC |
| | | TGCAGAAGGAAAACAGCAAGCGCTGGAACCCGGAGATCCAGTACACTTCCAACTATTAC |
| | | AAGTCTAATAATGTTGAATTTGCTGTTAATACTGAAGGTGTATATAGTGAACCCCGCCC |
| | | CATTGGCACCAGATACCTGACTCGTAATCTGTAA |
| AAV9 | 46 | MAADGYLPDWLEDNLSEGIREWWALKPGAPQPKANQQHQDNARGLVLPGYKYLGPGNGL |
| (amino acid) | | DKGEPVNAADAAALEHDKAYDQQLKAGDNPYLKYNHADAEFQERLKEDTSFGGNLGRAV |
| | | FQAKKRLLEPLGLVEEAAKTAPGKKRPVEQSPQEPDSSAGIGKSGAQPAKKRLNFGQTG |
| | | DTESVPDPQPIGEPPAAPSGVGSLTMASGGGAPVADNNEGADGVGSSSGNWHCDSQWLG |
| | | DRVITTSTRTWALPTYNNHLYKQISNSTSGGSSNDNAYFGYSTPWGYFDFNRFHCHFSP |
| | | RDWQRLINNNWGFRPKRLNFKLFNIQVKEVTDNNGVKTIANNLTSTVQVFTDSDYQLPY |
| | | VLGSAHEGCLPPFPADVFMIPQYGYLTLNDGSQAVGRSSFYCLEYFPSQMLRTGNNFQF |
| | | SYEFENVPFHSSYAHSQSLDRLMNPLIDQYLYYLSKTINGSGQNQQTLKFSVAGPSNMA |
| | | VQGRNYIPGPSYRQQRVSTTVTQNNNSEFAWPGASSWALNGRNSLMNPGPAMASHKEGE |
| | | DRFFPLSGSLIFGKQGTGRDNVDADKVMITNEEEIKTTNPVATESYGQVATNHQSAQAQ |
| | | AQTGWVQNQGILPGMVWQDRDVYLQGPIWAKIPHTDGNFHPSPLMGGFGMKHPPPQILI |
| | | KNTPVPADPPTAFNKDKLNSFITQYSTGQVSVEIEWELQKENSKRWNPEIQYTSNYYKS |
| | | NNVEFAVNTEGVYSEPRPIGTRYLTRNL |
| AAV9 | 47 | MSADGYLPDWLEDNLSEGIREWWALKPGAPQPKANQQHQDNARGLVLPGYKYLGPGNGL |
| (amino acid) | | DKGEPVNAADAAALEHDKAYDQQLKAGDNPYLKYNHADAEFQERLKEDTSFGGNLGRAV |
| | | FQAKKRLLEPLGLVEEAAKTAPGKKRPVEQSPQEPDSSAGIGKSGAQPAKKRLNFGQTG |
| | | DTESVPDPQPIGEPPAAPSGVGSLTMASGGGAPVADNNEGADGVGSSSGNWHCDSQWLG |
| | | DRVITTSTRTWALPTYNNHLYKQISNSTSGGSSNDNAYFGYSTPWGYFDFNRFHCHFSP |
| | | RDWQRLINNNWGFRPKRLNFKLFNIQVKEVTDNNGVKTIANNLTSTVQVFTDSDYQLPY |
| | | VLGSAHEGCLPPFPADVFMIPQYGYLTLNDGSQAVGRSSFYCLEYFPSQMLRTGNNFQF |
| | | SYEFENVPFHSSYAHSQSLDRLMNPLIDQYLYYLSKTINGSGQNQQTLKFSVAGPSNMA |
| | | VQGRNYIPGPSYRQQRVSTTVTQNNNSEFAWPGASSWALNGRNSLMNPGPAMASHKEGE |
| | | DRFFPLSGSLIFGKQGTGRDNVDADKVMITNEEEIKTTNPVATESYGQVATNHQSAQAQ |
| | | AQTGWVQNQGILPGMVWQDRDVYLQGPIWAKIPHTDGNFHPSPLMGGFGMKHPPPQILI |
| | | KNTPVPADPPTAFNKDKLNSFITQYSTGQVSVEIEWELQKENSKRWNPEIQYTSNYYKS |
| | | NNVEFAVNTEGVYSEPRPIGTRYLTRNL |
| AAV1 | 72 | atggctgccgatggttatcttccagattggctcgaggacaacctctctgagggcattcg |
| (DNA v1) | | cgagtggtgggacttgaaacctggagccccgaagcccaaagccaaccagcaaaagcagg |
| | | acgacggccggggtctggtgcttcctggctacaagtacctcggacccttcaacggactc |
| | | gacaagggggagcccgtcaacgcggcggacgcagcggccctcgagcacgacaaggccta |
| | | cgaccagcagctcaaagcgggtgacaatccgtacctgcggtataaccacgccgacgccg |
| | | agtttcaggagcgtctgcaagaagatacgtcttttgggggcaacctcgggcgagcagtc |
| | | ttccaggccaagaagcgggttctcgaacctctcggtctggttgaggaaggcgctaagac |
| | | ggctcctggaaagaaacgtccggtagagcagtcgccacaagagccagactcctcctcgg |
| | | gcatcggcaagacaggccagcagcccgctaaaaagagactcaattttggtcagactggc |
| | | gactcagagtcagtccccgatccacaacctctcggagaacctccagcaacccccgctgc |
| | | tgtgggacctactacaatggcttcaggcggtggcgcaccaatggcagacaataacgaag |
| | | gcgccgacggagtgggtaatgcctcaggaaattggcattgcgattccacatggctgggc |
| | | gacagagtcatcaccaccagcacccgcacctgggccttgcccacctacaataaccacct |
| | | ctacaagcaaatctccagtgcttcaacgggggccagcaacgacaaccactacttcggct |
| | | acagcaccccctgggggtattttgatttcaacagattccactgccacttttcaccacgt |
| | | gactggcagcgactcatcaacaacaattggggattccggcccaagagactcaacttcaa |
| | | actcttcaacatccaagtcaaggaggtcacgacgaatgatggcgtcacaaccatcgcta |
| | | ataaccttaccagcacggttcaagtcttctcggactcggagtaccagcttccgtacgtc |
| | | ctcggctctgcgcaccagggctgcctccctccgttcccggcggacgtgttcatgattcc |
| | | gcaatacggctacctgacgctcaacaatggcagccaagccgtgggacgttcatcctttt |
| | | actgcctggaatatttcccttctcagatgctgagaacgggcaacaactttaccttcagc |
| | | tacacctttgaggaagtgcctttccacagcagctacgcgcacagccagagcctggaccg |
| | | gctgatgaatcctctcatcgaccaatacctgtattacctgaacagaactcaaaatcagt |
| | | ccggaagtgcccaaaacaaggacttgctgtttagccgtgggtctccagctggcatgtct |
| | | gttcagcccaaaaactggctacctggaccctgttatcggcagcagcgcgtttctaaaac |
| | | aaaaacagacaacaacaacagcaattttacctggactggtgcttcaaaatataacctca |
| | | atgggcgtgaatccatcatcaaccctggcactgctatggcctcacacaaagacgacgaa |
| | | gacaagttctttcccatgagcggtgtcatgatttttggaaaagagagcgccggagcttc |
| | | aaacactgcattggacaatgtcatgattacagacgaagaggaaattaaagccactaacc |
| | | ctgtggccaccgaaagatttgggaccgtggcagtcaatttccagagcagcagcacagac |
| | | cctgcgaccggagatgtgcatgctatgggagcattacctggcatggtgtggcaagatag |
| | | agacgtgtacctgcagggtcccatttgggccaaaattcctcacacagatggacactttc |
| | | acccgtctcctcttatgggcggctttggactcaagaacccgcctcctcagatcctcatc |
| | | aaaaacacgcctgttcctgcgaatcctccggcggagttttcagctacaaagtttgcttc |
| | | attcatcacccaatactccacaggacaagtgagtgtggaaattgaatgggagctgcaga |
| | | aagaaaacagcaagcgctggaatcccgaagtgcagtacacatccaattatgcaaaatct |
| | | gccaacgttgattttactgtggacaacaatggactttatactgagcctcgccccattgg |
| | | cacccgttaccttacccgtcccctgtaa |
| AAV1 | 212 | ACGGCTGCCGACGGTTATCTACCCGATTGGCTCGAGGACAACCTCTCTGAGGGCATTCG |
| (DNA v2) | | CGAGTGGTGGGACTTGAAACCTGGAGCCCCGAAGCCCAAAGCCAACCAGCAAAAGCAGG |
| | | ACGACGGCCGGGGTCTGGTGCTTCCTGGCTACAAGTACCTCGGACCCTTCAACGGACTC |
| | | GACAAGGGGGAGCCCGTCAACGCGGCGGACGCAGCGGCCCTCGAGCACGACAAGGCCTA |
| | | CGACCAGCAGCTCAAAGCGGGTGACAATCCGTACCTGCGGTATAACCACGCCGACGCCG |
| | | AGTTTCAGGAGCGTCTGCAAGAAGATACGTCTTTTGGGGGCAACCTCGGGCGAGCAGTC |
| | | TTCCAGGCCAAGAAGCGGGTTCTCGAACCTCTCGGTCTGGTTGAGGAAGGCGCTAAGAC |
| | | GGCTCCTGGAAAGAAACGTCCGGTAGAGCAGTCGCCACAAGAGCCAGACTCCTCCTCGG |
| | | GCATCGGCAAGACAGGCCAGCAGCCCGCTAAAAAGAGACTCAATTTTGGTCAGACTGGC |
| | | GACTCAGAGTCAGTCCCCGATCCACAACCTCTCGGAGAACCTCCAGCAACCCCCGCTGC |
| | | TGTGGGACCTACTACAATGGCTTCAGGCGGTGGCGCACCAATGGCAGACAATAACGAAG |
| | | GCGCCGACGGAGTGGGTAATGCCTCAGGAAATTGGCATTGCGATTCCACATGGCTGGGC |
| | | GACAGAGTCATCACCACCAGCACCCGCACCTGGGCCTTGCCCACCTACAATAACCACCT |
| | | CTACAAGCAAATCTCCAGTGCTTCAACGGGGGCCAGCAACGACAACCACTACTTCGGCT |
| | | ACAGCACCCCCTGGGGGTATTTTGATTTCAACAGATTCCACTGCCACTTTTCACCACGT |
| | | GACTGGCAGCGACTCATCAACAACAATTGGGGATTCCGGCCCAAGAGACTCAACTTCAA |
| | | ACTCTTCAACATCCAAGTCAAGGAGGTCACGACGAATGATGGCGTCACAACCATCGCTA |
| | | ATAACCTTACCAGCACGGTTCAAGTCTTCTCGGACTCGGAGTACCAGCTTCCGTACGTC |
| | | CTCGGCTCTGCGCACCAGGGCTGCCTCCCTCCGTTCCCGGCGGACGTGTTCATGATTCC |
| | | GCAATACGGCTACCTGACGCTCAACAATGGCAGCCAAGCCGTGGGACGTTCATCCTTTT |
| | | ACTGCCTGGAATATTTCCCTTCTCAGATGCTGAGAACGGGCAACAACTTTACCTTCAGC |
| | | TACACCTTTGAGGAAGTGCCTTTCCACAGCAGCTACGCGCACAGCCAGAGCCTGGACCG |
| | | GCTGATGAATCCTCTCATCGACCAATACCTGTATTACCTGAACAGAACTCAAAATCAGT |
| | | CCGGAAGTGCCCAAAACAAGGACTTGCTGTTTAGCCGTGGGTCTCCAGCTGGCATGTCT |
| | | GTTCAGCCCAAAAACTGGCTACCTGGACCCTGTTATCGGCAGCAGCGCGTTTCTAAAAC |
| | | AAAAACAGACAACAACAACAGCAATTTTACCTGGACTGGTGCTTCAAAATATAACCTCA |
| | | ATGGGCGTGAATCCATCATCAACCCTGGCACTGCTATGGCCTCACACAAAGACGACGAA |
| | | GACAAGTTCTTTCCCATGAGCGGTGTCATGATTTTTGGAAAAGAGAGCGCCGGAGCTTC |
| | | AAACACTGCATTGGACAATGTCATGATTACAGACGAAGAGGAAATTAAAGCCACTAACC |
| | | CTGTGGCCACCGAAAGATTTGGGACCGTGGCAGTCAATTTCCAGAGCAGCAGCACAGAC |
| | | CCTGCGACCGGAGATGTGCATGCTATGGGAGCATTACCTGGCATGGTGTGGCAAGATAG |
| | | AGACGTGTACCTGCAGGGTCCCATTTGGGCCAAAATTCCTCACACAGATGGACACTTTC |
| | | ACCCGTCTCCTCTTATGGGCGGCTTTGGACTCAAGAACCCGCCTCCTCAGATCCTCATC |
| | | AAAAACACGCCTGTTCCTGCGAATCCTCCGGCGGAGTTTTCAGCTACAAAGTTTGCTTC |
| | | ATTCATCACCCAATACTCCACAGGACAAGTGAGTGTGGAAATTGAATGGGAGCTGCAGA |
| | | AAGAAAACAGCAAGCGCTGGAATCCCGAAGTGCAGTACACATCCAATTATGCAAAATCT |
| | | GCCAACGTTGATTTTACTGTGGACAACAATGGACTTTATACTGAGCCTCGCCCCATTGG |
| | | CACCCGTTACCTTACCCGTCCCCTGTAA |
| AAV1 | 71 | MAADGYLPDWLEDNLSEGIREWWDLKPGAPKPKANQQKQDDGRGLVLPGYKYLGPENGL |
| (amino acid) | | DKGEPVNAADAAALEHDKAYDQQLKAGDNPYLRYNHADAEFQERLQEDTSFGGNLGRAV |
| | | FQAKKRVLEPLGLVEEGAKTAPGKKRPVEQSPQEPDSSSGIGKTGQQPAKKRLNFGQTG |
| | | DSESVPDPQPLGEPPATPAAVGPTTMASGGGAPMADNNEGADGVGNASGNWHCDSTWLG |
| | | DRVITTSTRTWALPTYNNHLYKQISSASTGASNDNHYFGYSTPWGYFDFNRFHCHFSPR |
| | | DWQRLINNNWGFRPKRLNFKLFNIQVKEVTTNDGVTTIANNLTSTVQVFSDSEYQLPYV |
| | | LGSAHQGCLPPFPADVFMIPQYGYLTLNNGSQAVGRSSFYCLEYFPSQMLRTGNNFTFS |
| | | YTFEEVPFHSSYAHSQSLDRLMNPLIDQYLYYLNRTQNQSGSAQNKDLLFSRGSPAGMS |
| | | VQPKNWLPGPCYRQQRVSKTKTDNNNSNFTWIGASKYNLNGRESIINPGTAMASHKDDE |
| | | DKFFPMSGVMIFGKESAGASNTALDNVMITDEEEIKATNPVATERFGTVAVNFQSSSTD |
| | | PATGDVHAMGALPGMVWQDRDVYLQGPIWAKIPHTDGHFHPSPLMGGFGLKNPPPQILI |
| | | KNTPVPANPPAEFSATKFASFITQYSTGQVSVEIEWELQKENSKRWNPEVQYTSNYAKS |
| | | ANVDFTVDNNGLYTEPRPIGTRYLTRPL |
| AAV5 | 169 | atgtcttttgttgatcaccctccagattggttggaagaagttggtgaaggtcttcgcga |
| (DNA v1) | | gtttttgggccttgaagcgggcccaccgaaaccaaaacccaatcagcagcatcaagatc |
| | | aagcccgtggtcttgtgctgcctggttataactatctcggacccggaaacggtctcgat |
| | | cgaggagagcctgtcaacagggcagacgaggtcgcgcgagagcacgacatctcgtacaa |
| | | cgagcagcttgaggcgggagacaacccctacctcaagtacaaccacgcggacgccgagt |
| | | ttcaggagaagctcgccgacgacacatccttcgggggaaacctcggaaaggcagtcttt |
| | | caggccaagaaaagggttctcgaaccttttggcctggttgaagagggtgctaagacggc |
| | | ccctaccggaaagcggatagacgaccactttccaaaaagaaagaaggctcggaccgaag |
| | | aggactccaagccttccacctcgtcagacgccgaagctggacccagcggatcccagcag |
| | | ctgcaaatcccagcccaaccagcctcaagtttgggagctgatacaatgtctgcgggagg |
| | | tggcggcccattgggcgacaataaccaaggtgccgatggagtgggcaatgcctcgggag |
| | | attggcattgcgattccacgtggatgggggacagagtcgtcaccaagtccacccgaacc |
| | | tgggtgctgcccagctacaacaaccaccagtaccgagagatcaaaagcggctccgtcga |
| | | cggaagcaacgccaacgcctactttggatacagcaccccctgggggtactttgacttta |
| | | accgcttccacagccactggagcccccgagactggcaaagactcatcaacaactactgg |
| | | ggcttcagaccccggtccctcagagtcaaaatcttcaacattcaagtcaaagaggtcac |
| | | ggtgcaggactccaccaccaccatcgccaacaacctcacctccaccgtccaagtgttta |
| | | cggacgacgactaccagctgccctacgtcgtcggcaacgggaccgagggatgcctgccg |
| | | gccttccctccgcaggtctttacgctgccgcagtacggttacgcgacgctgaaccgcga |
| | | caacacagaaaatcccaccgagaggagcagcttcttctgcctagagtactttcccagca |
| | | agatgctgagaacgggcaacaactttgagtttacctacaactttgaggaggtgcccttc |
| | | cactccagcttcgctcccagtcagaacctgttcaagctggccaacccgctggtggacca |
| | | gtacttgtaccgcttcgtgagcacaaataacactggcggagtccagttcaacaagaacc |
| | | tggccgggagatacgccaacacctacaaaaactggttcccggggcccatgggccgaacc |
| | | cagggctggaacctgggctccggggtcaaccgcgccagtgtcagcgccttcgccacgac |
| | | caataggatggagctcgagggcgcgagttaccaggtgcccccgcagccgaacggcatga |
| | | ccaacaacctccagggcagcaacacctatgccctggagaacactatgatcttcaacagc |
| | | cagccggcgaacccgggcaccaccgccacgtacctcgagggcaacatgctcatcaccag |
| | | cgagagcgagacgcagccggtgaaccgcgtggcgtacaacgtcggcgggcagatggcca |
| | | ccaacaaccagagctcTACTACTGCCCCCGCGACCGGCACGTACAACCTCCAGGAAATC |
| | | GTGCCCGGCAGCGTGTGGATGGAGAGGGACGTGTACCTCCAAGGACCCATCTGGGCCAA |
| | | GATCCCAGAGACGGGGGCGCACTTTCACCCCTCTCCGGCCATGGGCGGATTCGGACTCA |
| | | AACACCCACCGCCCATGATGCTCATCAAGAACACGCCTGTGCCCGGAAATATCACCAGC |
| | | TTCTCGGACGTGCCCGTCAGCAGCTTCATCACCCAGTACAGCACCGGGCAGGTCACCGT |
| | | GGAGATGGAGTGGGAGCTCAAGAAGGAAAACTCCAAGAGGTGGAACCCAGAGATCCAGT |
| | | ACACAAACAACTACAACGACCCCCAGTTTGTGGACTTTGCCCCGGACAGCACCGGGGAA |
| | | TACAGAACCACCAGACCTATCGGAACCCGATACCTTACCCGACCCCTTTAA |
| AAV5 | 213 | AcGagTTTTGTTGATCACCCaCCCGATTGGTTGGAAGAAGTTGGTGAAGGTCTTCGCGA |
| (DNA v2) | | GTTTTTGGGCCTTGAAGCGGGCCCACCGAAACCAAAACCCAATCAGCAGCATCAAGATC |
| | | AAGCCCGTGGTCTTGTGCTGCCTGGTTATAACTATCTCGGACCCGGAAACGGTCTCGAT |
| | | CGAGGAGAGCCTGTCAACAGGGCAGACGAGGTCGCGCGAGAGCACGACATCTCGTACAA |
| | | CGAGCAGCTTGAGGCGGGAGACAACCCCTACCTCAAGTACAACCACGCGGACGCCGAGT |
| | | TTCAGGAGAAGCTCGCCGACGACACATCCTTCGGGGGAAACCTCGGAAAGGCAGTCTTT |
| | | CAGGCCAAGAAAAGGGTTCTCGAACCTTTTGGCCTGGTTGAAGAGGGTGCTAAGACGGC |
| | | CCCTACCGGAAAGCGGATAGACGACCACTTTCCAAAAAGAAAGAAGGCCCGGACCGAAG |
| | | AGGACTCCAAGCCTTCCACCTCGTCAGACGCCGAAGCTGGACCCAGCGGATCCCAGCAG |
| | | CTGCAAATCCCAGCCCAACCAGCCTCAAGTTTGGGAGCTGATACAATGTCTGCGGGAGG |
| | | TGGCGGCCCATTGGGCGACAATAACCAAGGTGCCGATGGAGTGGGCAATGCCTCGGGAG |
| | | ATTGGCATTGCGATTCCACGTGGATGGGGGACAGAGTCGTCACCAAGTCCACCCGAACC |
| | | TGGGTGCTGCCCAGCTACAACAACCACCAGTACCGAGAGATCAAAAGCGGCTCCGTCGA |
| | | CGGAAGCAACGCCAACGCCTACTTTGGATACAGCACCCCCTGGGGGTACTTTGACTTTA |
| | | ACCGCTTCCACAGCCACTGGAGCCCCCGAGACTGGCAAAGACTCATCAACAACTACTGG |
| | | GGCTTCAGACCCCGGTCCCTCAGAGTCAAAATCTTCAACATTCAAGTCAAAGAGGTCAC |
| | | GGTGCAGGACTCCACCACCACCATCGCCAACAACCTCACCTCCACCGTCCAAGTGTTTA |
| | | CGGACGACGACTACCAGCTGCCCTACGTCGTCGGCAACGGGACCGAGGGATGCCTGCCG |
| | | GCCTTCCCTCCGCAGGTCTTTACGCTGCCGCAGTACGGTTACGCGACGCTGAACCGCGA |
| | | CAACACAGAAAATCCCACCGAGAGGAGCAGCTTCTTCTGCCTAGAGTACTTTCCCAGCA |
| | | AGATGCTGAGAACGGGCAACAACTTTGAGTTTACCTACAACTTTGAGGAGGTGCCCTTC |
| | | CACTCCAGCTTCGCTCCCAGTCAGAACCTCTTCAAGCTGGCCAACCCGCTGGTGGACCA |
| | | GTACTTGTACCGCTTCGTGAGCACAAATAACACTGGCGGAGTCCAGTTCAACAAGAACC |
| | | TGGCCGGGAGATACGCCAACACCTACAAAAACTGGTTCCCGGGGCCCATGGGCCGAACC |
| | | CAGGGCTGGAACCTGGGCTCCGGGGTCAACCGCGCCAGTGTCAGCGCCTTCGCCACGAC |
| | | CAATAGGATGGAGCTCGAGGGCGCGAGTTACCAGGTGCCCCCGCAGCCGAACGGCATGA |
| | | CCAACAACCTCCAGGGCAGCAACACCTATGCCCTGGAGAACACTATGATCTTCAACAGC |
| | | CAGCCGGCGAACCCGGGCACCACCGCCACGTACCTCGAGGGCAACATGCTCATCACCAG |
| | | CGAGAGCGAGACGCAGCCGGTGAACCGCGTGGCGTACAACGTCGGCGGGCAGATGGCCA |
| | | CCAACAACCAGAGCTCCACCACTGCCCCCGCGACCGGCACGTACAACCTCCAGGAAATC |
| | | GTGCCCGGCAGCGTGTGGATGGAGAGGGACGTGTACCTCCAAGGACCCATCTGGGCCAA |
| | | GATCCCAGAGACGGGGGCGCACTTTCACCCCTCTCCGGCCATGGGCGGATTCGGACTCA |
| | | AACACCCACCGCCCATGATGCTCATCAAGAACACGCCTGTGCCCGGAAATATCACCAGC |
| | | TTCTCGGACGTGCCCGTCAGCAGCTTCATCACCCAGTACAGCACCGGGCAGGTCACCGT |
| | | GGAGATGGAGTGGGAGCTCAAGAAGGAAAACTCCAAGAGGTGGAACCCAGAGATCCAGT |
| | | ACACAAACAACTACAACGACCCCCAGTTTGTGGACTTTGCCCCGGACAGCACCGGGGAA |
| | | TACAGAACCACCAGACCTATCGGAACCCGATACCTTACCCGACCCCTTTAA |
| AAV5 | 168 | MSFVDHPPDWLEEVGEGLREFLGLEAGPPKPKPNQQHQDQARGLVLPGYNYLGPGNGLD |
| (amino acid) | | RGEPVNRADEVAREHDISYNEQLEAGDNPYLKYNHADAEFQEKLADDTSFGGNLGKAVF |
| | | QAKKRVLEPFGLVEEGAKTAPTGKRIDDHFPKRKKARTEEDSKPSTSSDAEAGPSGSQQ |
| | | LQIPAQPASSLGADTMSAGGGGPLGDNNQGADGVGNASGDWHCDSTWMGDRVVTKSTRT |
| | | WVLPSYNNHQYREIKSGSVDGSNANAYFGYSTPWGYFDFNRFHSHWSPRDWQRLINNYW |
| | | GFRPRSLRVKIFNIQVKEVTVQDSTTTIANNLTSTVQVFTDDDYQLPYVVGNGTEGCLP |
| | | AFPPQVFTLPQYGYATLNRDNTENPTERSSFFCLEYFPSKMLRIGNNFEFTYNFEEVPF |
| | | HSSFAPSQNLFKLANPLVDQYLYRFVSTNNTGGVQFNKNLAGRYANTYKNWFPGPMGRT |
| | | QGWNLGSGVNRASVSAFATTNRMELEGASYQVPPQPNGMINNLQGSNTYALENTMIENS |
| | | QPANPGTTATYLEGNMLITSESETQPVNRVAYNVGGQMAINNQSSTTAPATGTYNLQEI |
| | | VPGSVWMERDVYLQGPIWAKIPETGAHFHPSPAMGGFGLKHPPPMMLIKNTPVPGNITS |
| | | FSDVPVSSFITQYSTGQVTVEMEWELKKENSKRWNPEIQYTNNYNDPQFVDFAPDSTGE |
| | | YRTTRPIGTRYLTRPL |

In some embodiments, the VP-coding region is operably linked to a promoter. In some embodiments, the promoter is a baculovirus major late promoter, a viral promoter, an insect viral promoter, a non-insect viral promoter, a vertebrate viral promoter, a chimeric promoter from one or more species including virus and non-virus elements, a synthetic promoter, or a variant thereof. In some embodiments, the promoter is chosen from a polh promoter, a p10 promoter, a Ctx promoter, a gp64 promoter, an IE promoter, an IE-1 promoter, a p6.9 promoter, a Dmhsp70 promoter, a Hsp70 promoter, a p5 promoter, a p19 promoter, a p35 promoter, a p40 promoter, or a variant, e.g., functional fragment, thereof. In some embodiments, the promoter is a p10 promoter. In some embodiments, the promoter comprises a nucleotide sequence provided in Table 3 or a nucleotide sequence at least 70%, 75%, 80%, 85%, 90%, 95%, 96%, 97%, 98%, or 99% identical thereto. In some embodiments, the promoter comprises the nucleotide sequence of SEQ ID NO: 200, or a nucleotide sequence at least 70%, 75%, 80%, 85%, 90%, 95%, 96%, 97%, 98%, or 99% identical thereto. In some embodiments, the promoter is a p10 promoter and comprises the nucleotide sequence of SEQ ID NO: 200, or a nucleotide sequence at least 70%, 75%, 80%, 85%, 90%, 95%, 96%, 97%, 98%, or 99% identical thereto. In some embodiments, the promoter is a ctx promoter. In some embodiments, the CTX promoter comprises a sequence as provided in Table 3, or a sequence substantially identical (e.g., having at least about 70%, 75%, 80%, 85%, 90%, 92%, 95%, 97%, 98%, or 99% sequence identity) thereto.

In some embodiments, the VP-coding region is present in the SOD gene locus of the variant baculovirus genome and is operably linked to a p10 promoter. In some embodiments, the VP-coding region is present in the SOD gene locus of a variant baculovirus genome and comprises a single polycistronic ORF encoding a VP1 protein, a VP2 protein, and a VP3 protein. In some embodiments, the VP-coding region is present in the SOD gene locus of the variant baculovirus genome and comprises a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein.

In some embodiments, the viral (e.g., AAV) expression construct comprises in 5′ to 3′ order, a p10 promoter and the VP-coding region comprising a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein, wherein the VP-coding region is present in the SOD gene locus of the variant baculovirus genome.

In some embodiments, the VP-coding region is present in the SOD gene locus of the variant baculovirus genome and comprises a single polycistronic ORF encoding a VP1 protein, a VP2 protein, and a VP3 protein, wherein the ORF encoding the VP1 protein comprises an ACG or ATG start codon, the ORF encoding the VP2 protein comprises an ACG start codon, and the ORF encoding the VP3 protein comprises an ATG start codon.

TABLE 3
Exemplary Promoter Sequences

| | SEQ | |
|---|---|---|
| | ID | |
| Name | NO | Sequence |
| polh | 167 | ATCATGGAGATAATTAAAATGATAACCATCTCGCAAATAAATAAGTATTTTACTGTTTTC |
| Promoter 1 | | GTAACAGTTTTGTAATAAAAAAACCTATAAAT |
| polh | 220 | ATCtTGGAGATAATTAAAtTGATAACCATCTCGCAAATAAATAAGTATTTTACT |
| Promoter 2 | | GTTTTCGTAACAGTTTTGTAATAAAAAAACCTATAAAT |
| p10 | 200 | GACCTTTAATTCAACCCAACACAATATATTATAGTTAAATAAGAATTATTATCAAATCAT |
| promoter | | TTGTATATTAATTAAAATACTATACTGTAAATTACATTTTA |
| gp64 | 217 | AAATTATCGCAAGATAAGGCGCACGTTGATTGGGTCACCCGAGTGTACGTTGATAAAGTC |
| promoter | | ACGTGGGCACCCAACGCGTTGATAAGCATCGGTATATAAGGGCCTACAGTGTTCTGGTAA |
| | | ATCAGTTGCACTGTGCTCTTCACAGGAACACTACAAGACCTACAAG |
| CTX | 164 | tgaaactaacttacaagTtggctagtttgttaaaatacgcgctgcgcttgactcgggaat |
| promoter | | acaaagaaaacattattccacactttgatcacttgactcgattgcgcgatttaatcgacg |
| | | gcTtgattaaaagcgaggatgtacaacgttttaatcgcactaatcgcaatgatttaattt |
| | | cggcttgcTtgcaaatcaacgttcggacgtacTtgcccaacgccacgatagatTtgcgca |
| | | aacaacccaactgtatatattttcgaatttgccaatattgccacttggaggccgacgtgc |
| | | cttcgcccgacgatcattcggtgtacagatacttgtgcgtcgcgtgcggcacgccgctgg |
| | | tcatcgaccacccgctcgacgtgttcggccacacggaggaaggcgtcaacgaactgctcg |
| | | aggtgcagcgagtcaacgcgggcggggagttgtaggcgtcataactatttattaaATAAG |
| | | ataatttaaaaaatcgccgttaat |
| CTX | 165 | ATAAGataatttaaaaaatcgccgttaat |
| promoter | | |
| CTX | 166 | gggcggggagttgtaggcgtcataactatttattaaATAAGataatttaaaaaatcgccg |
| promoter | | ttaat |
| gp64-polh | 21 | aaattatcgcaagATAAGgcgcacgttgattgggtcacccgagtgtacgttgat |
| promoter | | aaagtcacgtgggcacccaacgcgttgATAAGcatCggTATATAAgggcctaca |
| | | gtgttctggtaaatCAGTtgcactgtgctcttcacaggaacactacaagaccta |
| | | caagGctagcgtatacATCtTGGAGATAATTAAAtTGATAACCATCTCGCAAAT |
| | | AAATAAGTATTTTACTGTTTTCGTAACAGTTTTGTAATAAAAAAACCTATAAAT |
| | | Agatctaccgcc |

In some embodiments, a baculovirus expression construct or a variant baculovirus genome described herein comprises an AAV expression construct comprising a Rep-coding region. In some embodiments, an AAV expression construct can comprise coding regions for Rep52, Rep78, Rep40, and/or Rep68 proteins. In some embodiments, the AAV expression construct comprises a first nucleotide sequence which comprises a Rep52-coding region, a Rep78-coding region, or a combination thereof. In some embodiments, the first nucleotide sequence comprises a single open reading frame. In some embodiments, the first nucleotide sequence comprises a first open reading frame which comprises a Rep52-coding region, and a second open reading frame which comprises a Rep78-coding region and which is different from the first open reading frame.

In some embodiments, a Rep-coding region in an AAV expression construct described herein comprises a nucleotide sequence in Table 4, or encodes a Rep protein comprising an amino acid sequence as provided in Table 4, or a sequence substantially identical (e.g., having at least 70%, 75%, 80%, 85%, 90%, 95%, 96%, 97%, 98%, or 99%) thereto. In some embodiments, the nucleotide sequence encoding the Rep52 comprises nucleotides 673-1866 of SEQ ID NO: 201. In some embodiments, the encoded Rep52 protein comprises amino acids 225-621 of SEQ ID NO: 202. In some embodiments, the Rep-coding region comprises the nucleotide sequence of SEQ ID NO: 201, or a nucleotide sequence at least 70%, 75%, 80%, 85%, 90%, 95%, 96%, 97%, 98%, or 99% identical thereto; a nucleotide sequence having at least 10, 20, 50, 100, 150, 200, 250, 300, 350, 400, or 450 but no more than 500 different nucleotides relative to SEQ ID NO: 201; or a nucleotide sequence having at least 10, 20, 50, 100, 150, 200, 250, 300, 350, 400, or 450 but no more than 500 modifications (e.g., substitutions) relative to SEQ ID NO: 201. In some embodiments, the Rep-coding region encodes the amino acid sequence of SEQ ID NO: 202; an amino acid sequence at least 70%, 75%, 80%, 85%, 90%, 95%, 96%, 97%, 98%, or 99% identical thereto; an amino acid sequence comprising at least 1, 2, 3, 4, 5, 10, 15, or 20 but no more than 30 different amino acids relative to SEQ ID NO: 202; or an amino acid sequence comprising at least 1, 2, 3, 4, 5, 10, 15, or 20 but no more than 30 modifications (e.g., substitutions (e.g., conservative substitutions), insertions, or deletions) relative to the amino acid sequence of SEQ ID NO: 202.

TABLE 4
Exemplary full length Rep sequences

| | SEQ | |
|---|---|---|
| | ID | |
| Description | NO: | Sequence |
| Rep78 | 201 | ATGGCGGGGTTTTACGAGATTGTGATTAAGGTCCCCAGCGACCTTGACGAGCATCTGCC |
| (DNA) | | CGGCATTTCTGACAGCTTTGTGAACTGGGTGGCCGAGAAGGAGTGGGAGTTGCCGCCAG |
| | | ATTCTGACTTGGATCTGAATCTGATTGAGCAGGCACCCCTGACCGTGGCCGAGAAGCTG |
| | | CAGCGCGACTTTCTGACGGAGTGGCGCCGTGTGAGTAAGGCCCCGGAGGCCCTTTTCTT |
| | | TGTGCAATTTGAGAAGGGAGAGAGCTACTTCCACTTACACGTGCTCGTGGAAACCACCG |
| | | GGGTGAAATCCTTAGTTTTGGGACGTTTCCTGAGTCAGATTCGCGAAAAACTGATTCAG |
| | | AGAATTTACCGCGGGATCGAGCCGACTTTGCCAAACTGGTTCGCGGTCACAAAGACCAG |
| | | AAACGGCGCCGGAGGCGGGAACAAGGTGGTGGACGAGTGCTACATCCCCAATTACTTGC |
| | | TCCCCAAAACCCAGCCTGAGCTCCAGTGGGCGTGGACTAATTTAGAACAGTATTTAAGC |
| | | GCCTGTTTGAATCTCACGGAGCGTAAACGGTTGGTGGCGCAGCATCTGACGCACGTGTC |
| | | GCAGACGCAGGAGCAGAACAAAGAGAATCAGAATCCCAATTCTGACGCGCCGGTGATCA |
| | | GATCAAAAACTTCAGCCAGaTACATGGAGCTGGTCGGGTGGCTCGTGGACAAGGGGATT |
| | | ACCTCGGAGAAGCAGTGGATCCAGGAGGACCAGGCCTCATACATCTCCTTCAATGCGGC |
| | | CTCCAACTCGCGGTCCCAAATCAAGGCTGCCTTGGACAATGCGGGAAAGATTATGAGCC |
| | | TGACTAAAACCGCCCCCGACTACCTGGTGGGCCAGCAGCCCGTGGAGGACATTTCCAGC |
| | | AATCGGATTTATAAAATTTTGGAACTAAACGGGTACGATCCCCAATATGCGGCTTCCGT |
| | | CTTTCTGGGATGGGCCACGAAAAAGTTCGGCAAGAGGAACACCATCTGGCTGTTTGGGC |
| | | CTGCAACTACCGGGAAGACCAACATCGCGGAGGCCATAGCCCACACTGTGCCCTTCTAC |
| | | GGGTGCGTAAACTGGACCAATGAGAACTTTCCCTTCAACGACTGTGTCGACAAGATGGT |
| | | GATCTGGTGGGAGGAGGGGAAGATGACCGCCAAGGTCGTGGAGTCGGCCAAAGCCATTC |
| | | TCGGAGGAAGCAAGGTGCGCGTGGACCAGAAATGCAAGTCCTCGGCCCAGATAGACCCG |
| | | ACTCCCGTGATCGTCACCTCCAACACCAACATGTGCGCCGTGATTGACGGGAACTCAAC |
| | | GACCTTCGAACACCAGCAGCCGTTGCAAGACCGGATGTTCAAATTTGAACTCACCCGCC |
| | | GTCTGGATCATGACTTTGGGAAGGTCACCAAGCAGGAAGTCAAAGACTTTTTCCGGTGG |
| | | GCAAAGGATCACGTGGTTGAGGTGGAGCATGAATTCTACGTCAAAAAGGGTGGAGCCAA |
| | | GAAAAGACCCGCCCCCAGTGACGCAGATATAAGTGAGCCCAAACGGGTGCGCGAGTCAG |
| | | TTGCGCAGCCATCGACGTCAGACGCGGAAGCTTCGATCAACTACGCAGACAGGTACCAA |
| | | AACAAATGTTCTCGTCACGTGGGCATGAATCTGATGCTGTTTCCCTGCAGACAATGCGA |
| | | GAGAATGAATCAGAATTCAAATATCTGCTTCACTCACGGACAGAAAGACTGTTTAGAGT |
| | | GCTTTCCCGTGTCAGAATCTCAACCCGTTTCTGTCGTCAAAAAGGCGTATCAGAAACTG |
| | | TGCTACATTCATCATATCATGGGAAAGGTGCCAGACGCTTGCACTGCCTGCGATCTGGT |
| | | CAATGTGGATTTGGATGACTGCATCTTTGAACAATAA |
| Rep78 | 202 | MAGFYEIVIKVPSDLDEHLPGISDSFVNWVAEKEWELPPDSDLDLNLIEQAPLTVAEKL |
| (amino acid) | | QRDFLTEWRRVSKAPEALFFVQFEKGESYFHLHVLVETTGVKSLVLGRFLSQIREKLIQ |
| | | RIYRGIEPTLPNWFAVTKTRNGAGGGNKVVDECYIPNYLLPKTQPELQWAWINLEQYLS |
| | | ACLNLTERKRLVAQHLTHVSQTQEQNKENQNPNSDAPVIRSKTSARYMELVGWLVDKGI |
| | | TSEKQWIQEDQASYISFNAASNSRSQIKAALDNAGKIMSLTKTAPDYLVGQQPVEDISS |
| | | NRIYKILELNGYDPQYAASVFLGWATKKFGKRNTIWLFGPATTGKINIAEAIAHTVPFY |
| | | GCVNWTNENFPFNDCVDKMVIWWEEGKMTAKVVESAKAILGGSKVRVDQKCKSSAQIDP |
| | | TPVIVTSNTNMCAVIDGNSTTFEHQQPLQDRMFKFELTRRLDHDFGKVTKQEVKDFFRW |
| | | AKDHVVEVEHEFYVKKGGAKKRPAPSDADISEPKRVRESVAQPSTSDAEASINYADRYQ |
| | | NKCSRHVGMNLMLFPCRQCERMNQNSNICFTHGQKDCLECFPVSESQPVSVVKKAYQKL |
| | | CYIHHIMGKVPDACTACDLVNVDLDDCIFEQ |
| Rep52 | 203 | ATGGAGCTGGTCGGGTGGCTCGTGGACAAGGGGATTACCTCGGAGAAGCAGTGGATCCA |
| (DNA) | | GGAGGACCAGGCCTCATACATCTCCTTCAATGCGGCCTCCAACTCGCGGTCCCAAATCA |
| | | AGGCTGCCTTGGACAATGCGGGAAAGATTATGAGCCTGACTAAAACCGCCCCCGACTAC |
| | | CTGGTGGGCCAGCAGCCCGTGGAGGACATTTCCAGCAATCGGATTTATAAAATTTTGGA |
| | | ACTAAACGGGTACGATCCCCAATATGCGGCTTCCGTCTTTCTGGGATGGGCCACGAAAA |
| | | AGTTCGGCAAGAGGAACACCATCTGGCTGTTTGGGCCTGCAACTACCGGGAAGACCAAC |
| | | ATCGCGGAGGCCATAGCCCACACTGTGCCCTTCTACGGGTGCGTAAACTGGACCAATGA |
| | | GAACTTTCCCTTCAACGACTGTGTCGACAAGATGGTGATCTGGTGGGAGGAGGGGAAGA |
| | | TGACCGCCAAGGTCGTGGAGTCGGCCAAAGCCATTCTCGGAGGAAGCAAGGTGCGCGTG |
| | | GACCAGAAATGCAAGTCCTCGGCCCAGATAGACCCGACTCCCGTGATCGTCACCTCCAA |
| | | CACCAACATGTGCGCCGTGATTGACGGGAACTCAACGACCTTCGAACACCAGCAGCCGT |
| | | TGCAAGACCGGATGTTCAAATTTGAACTCACCCGCCGTCTGGATCATGACTTTGGGAAG |
| | | GTCACCAAGCAGGAAGTCAAAGACTTTTTCCGGTGGGCAAAGGATCACGTGGTTGAGGT |
| | | GGAGCATGAATTCTACGTCAAAAAGGGTGGAGCCAAGAAAAGACCCGCCCCCAGTGACG |
| | | CAGATATAAGTGAGCCCAAACGGGTGCGCGAGTCAGTTGCGCAGCCATCGACGTCAGAC |
| | | GCGGAAGCTTCGATCAACTACGCAGACAGgtaccaaaacaaatgttctcgtcacgtggg |
| | | catgaatctgatgctgtttccctgcagacaatgcgagagaatgaatcagaattcaaata |
| | | tctgcttcactcacggacagaaagactgtttagagtgctttcccgtgtcagaatctcaa |
| | | cccgtttctgtcgtcaaaaaggcgtatcagaaactgtgctacattcatcatatcatggg |
| | | aaaggtgccagacgcttgcactgcctgcgatctggtcaatgtggatttggatgactgca |
| | | tctttgaacaataa |
| Rep52 | 204 | MELVGWLVDKGITSEKQWIQEDQASYISFNAASNSRSQIKAALDNAGKIMSLIKTAPDY |
| (amino acid) | | LVGQQPVEDISSNRIYKILELNGYDPQYAASVELGWATKKFGKRNTIWLFGPATTGKIN |
| | | IAEAIAHTVPFYGCVNWTNENFPFNDCVDKMVIWWEEGKMTAKVVESAKAILGGSKVRV |
| | | DQKCKSSAQIDPTPVIVISNINMCAVIDGNSTTFEHQQPLQDRMFKFELTRRLDHDFGK |
| | | VTKQEVKDFFRWAKDHVVEVEHEFYVKKGGAKKRPAPSDADISEPKRVRESVAQPSTSD |
| | | AEASINYADRYQNKCSRHVGMNLMLFPCRQCERMNQNSNICFTHGQKDCLECFPVSESQ |
| | | PVSVVKKAYQKLCYIHHIMGKVPDACTACDLVNVDLDDCIFEQ |

In some embodiments, the Rep-coding region is operably linked to a promoter. In some embodiments, the promoter is a baculovirus major late promoter, a viral promoter, an insect viral promoter, a non-insect viral promoter, a vertebrate viral promoter, a chimeric promoter from one or more species including virus and non-virus elements, a synthetic promoter, or a variant thereof. In some embodiments, the promoter is chosen from a polh promoter, a p10 promoter, a Ctx promoter, a gp64 promoter, an IE promoter, an IE-1 promoter, a p6.9 promoter, a Dmhsp70 promoter, a Hsp70 promoter, a p5 promoter, a p19 promoter, a p35 promoter, a p40 promoter, or a variant, e.g., functional fragment, thereof. In some embodiments, the promoter is a p10 promoter. In some embodiments, the promoter comprises a nucleotide sequence provided in Table 3 or a nucleotide sequence at least 70%, 75%, 80%, 85%, 90%, 95%, 96%, 97%, 98%, or 99% identical thereto. In some embodiments, the promoter comprises the nucleotide sequence of SEQ ID NO: 200, or a nucleotide sequence at least 70%, 75%, 80%, 85%, 90%, 95%, 96%, 97%, 98%, or 99% identical thereto. In some embodiments, the promoter is a p10 promoter and comprises the nucleotide sequence of SEQ ID NO: 200, or a nucleotide sequence at least 70%, 75%, 80%, 85%, 90%, 95%, 96%, 97%, 98%, or 99% identical thereto. In some embodiments, the promoter is a ctx promoter. In some embodiments, the CTX promoter comprises a sequence as provided in Table 3, or a sequence substantially identical (e.g., having at least about 70%, 75%, 80%, 85%, 90%, 92%, 95%, 97%, 98%, or 99% sequence identity) thereto.

In some embodiments, the viral expression construct comprises a Rep78-coding region in a first transcriptional cassette (e.g., ORF). In some embodiments, the viral expression construct comprises a Rep52-coding region in a second transcriptional cassette (e.g., ORF), which is separate from the first transcriptional cassette comprising the Rep78-coding region. In some embodiments, the first transcriptional cassette is at a first location of a AAV expression construct, and the second transcriptional cassette is at a second location of the AAV expression construct. In some embodiments, the first transcriptional cassette is in the polh gene location of the AAV expression construct. In some embodiments, the first transcriptional cassette is in the egt gene location of the AAV expression construct. In some embodiments, the second transcriptional cassette is in the polh gene locus of the AAV expression construct. In some embodiments, the second transcriptional cassette is in the egt gene locus of the AAV expression construct. In some embodiments, the first transcriptional cassette is in the polh gene locus and the second transcriptional cassette is in the egt gene locus of the AAV expression vector. In some embodiments, the first transcriptional cassette is in the egt gene locus and the second transcriptional cassette is in the polh gene locus of the AAV expression vector. In some embodiments, the Rep-coding region is present in the p74 gene location of the viral (AAV) expression vector.

In some embodiments, non-structural proteins, Rep52 and Rep78, can be encoded in a single open reading frame regulated by utilization of both alternative splice acceptor and non-canonical translational initiation codons.

Both Rep78 and Rep52 can be translated from a single transcript: Rep78 translation initiates at a first start codon (AUG or non-AUG) and Rep52 translation initiates from a Rep52 start codon (e.g., AUG) within the Rep78 sequence. Rep78 and Rep52 can also be translated from separate transcripts with independent start codons. The Rep52 initiation codons within the Rep78 sequence can be mutated, modified, or removed, such that processing of the modified Rep78 sequence will not produce Rep52 proteins. In some embodiments, a single coding sequence is used for the Rep78 and Rep52 proteins, wherein start codon for translation of the Rep78 protein is a suboptimal start codon, selected from the group consisting of ACG, TTG, CTG and GTG, that effects partial exon skipping upon expression in insect cells, as described in U.S. Pat. No. 8,512,981, the content of which is incorporated herein by reference. In some embodiments, (i) the ORF encoding the Rep78 protein comprises an ATG start codon and the ORF encoding the Rep52 protein comprises an ATG start codon; (ii) the ORF encoding the Rep78 protein comprises an CTG start codon and the ORF encoding the Rep52 protein comprises an ATG start codon; (iii) the ORF encoding the Rep78 protein comprises an ATG start codon and the ORF encoding the Rep52 protein comprises an CTG start codon; or (iv) the ORF encoding the Rep78 protein comprises an CTG start codon and the ORF encoding the Rep52 protein comprises an CTG start codon.

In some embodiments, the Rep-coding region comprises a nucleotide sequence encoding (i) a Rep78 protein and a Rep52 protein; (ii) primarily a Rep78 protein, e.g., at least 50%, 60%, 70%, 80%, 90% or more Rep78 protein relative to a Rep52 protein; (iii) a Rep78 protein only; (iv) a Rep78 protein, but not a Rep52 protein; (v) a Rep52 protein only; or (vi) a Rep52 protein, but not a Rep78 protein. In some embodiments, the Rep-coding region comprises a nucleotide sequence encoding a Rep78 protein and a Rep52 protein, wherein the nucleotide sequence encoding the Rep52 protein is comprised within the nucleotide sequence encoding the Rep78 protein. In some embodiments, the Rep-coding region comprises a single polycistronic ORF encoding a Rep78 protein and a Rep52 protein.

In some embodiments, the AAV expression construct may contain repeating codons with differential codon biases, for example to achieve improved ratios of Rep proteins, e.g., Rep78 and Rep52 thereby improving large scale (commercial) production of viral expression construct and/or payload construct vectors in insect cells, as taught in U.S. Pat. No. 8,697,417, the content of which is incorporated herein by reference in its entirety. In some embodiment, improved ratios of rep proteins may be achieved using the method and constructs described in U.S. Pat. No. 8,642,314, the contents of which are incorporated herein by reference in its entirety.

In some embodiments, the AAV expression construct may encode mutant parvoviral Rep polypeptides which have one or more improved properties as compared with their corresponding wild-type Rep polypeptide, such as the preparation of higher virus titers for large scale production. Alternatively, they may be able to allow the production of better-quality viral particles or sustain more stable production of virus. In a non-limiting example, the viral expression construct may encode mutant Rep polypeptides with a mutated nuclear localization sequence or zinc finger domain, as described in US2013/0023034, the contents of which are incorporated herein by reference in its entirety.

In some embodiments, the AAV expression construct comprises a first nucleotide sequence and a separate second nucleotide sequence, wherein the first nucleotide sequence comprises a Rep52-coding region and a 2A sequence region, and wherein the second nucleotide sequence comprises a Rep78-coding region and a 2A sequence region. In some embodiments, a first nucleotide sequence comprises a Rep52-coding region and 2A sequence region. In some embodiments, a first nucleotide sequence comprises a Rep78-coding region and 2A sequence region. In some embodiments, a first nucleotide sequence comprises a Rep52-coding region, a Rep78-coding region, and 2A sequence region. In some embodiments, a first nucleotide sequence comprises a 2A sequence region located between a Rep52-coding region and a Rep78-coding region on the nucleotide sequence. In some embodiments, a first nucleotide comprises, in order from the 5′-end to the 3′-end, a Rep52-coding region, a 2A sequence region, and a Rep78-coding region. In some embodiments, a first nucleotide comprises, in order from the 5′-end to the 3′-end, a Rep78-coding region, a 2A sequence region, and a Rep52-coding region.

In some embodiments, a first nucleotide sequence comprises a Rep52-coding region, a Rep78-coding region, and an IRES sequence region. In some embodiments, a first nucleotide sequence comprises an IRES sequence region located between a Rep52-coding region and a Rep78-coding region on the nucleotide sequence. In some embodiments, a first nucleotide comprises, in order from the 5′-end to the 3′-end, a Rep52-coding region, an IRES sequence region, and a Rep78-coding region. In certain embodiments, a first nucleotide comprises, in order from the 5′-end to the 3′-end, a Rep78-coding region, an IRES sequence region, and a Rep52-coding region. In some embodiments, the first nucleotide sequence comprises a first open reading frame which comprises a Rep52-coding region, a second open reading frame which comprises a Rep78-coding region, and an IRES sequence region located between the first open reading frame and the second open reading frame. In some embodiments, a first nucleotide sequence comprises, in order from the 5′-end to the 3′-end, a first open reading frame which comprises a Rep52-coding region, an IRES sequence region, and a second open reading frame which comprises a Rep78-coding region. In some embodiments, a first nucleotide sequence comprises, in order from the 5′-end to the 3′-end, a first open reading frame which comprises a Rep78-coding region, an IRES sequence region, and a second open reading frame which comprises a Rep52-coding region.

In some embodiments, Rep52 or Rep78 is transcribed from the baculovirus-derived polyhedron promoter (polh). Rep52 or Rep78 can also be transcribed from a weaker promoter, for example, a deletion mutant of the IE-1 promoter (AIE-1 promoter), which has about 20% of the transcriptional activity of that IE-1 promoter.

In some embodiments, the baculovirus expression constructs or variant baculovirus genomes described herein comprise an AAV expression construct comprising a Rep-coding region which comprises a nucleotide sequence encoding a Rep protein chosen from Rep52, Rep40, Rep68, Rep78, or a combination thereof (e.g., a Rep52 protein and/or a Rep78 protein), wherein the Rep-coding region is operably linked to a first promoter and optionally a second promoter. In some embodiments, the Rep-coding region is operably linked to both a first promoter and a second promoter.

In some embodiments, the first and/or second promoter is selected from a baculovirus promoter, a viral promoter, an insect viral promoter, a non-insect viral promoter, a vertebrate viral promoter, a chimeric promoter from one or more species including virus and non-virus elements, a synthetic promoter, or a variant thereof. In some embodiments, the first and/or second promoter chosen from a polh promoter, a p10 promoter, a ctx promoter, a gp64 promoter, an IE promoter, an IE-1 promoter, a p6.9 promoter, a Dmhsp70 promoter, a Hsp70 promoter, a p5 promoter, a p19 promoter, a p35 promoter, a p40 promoter, or a variant, e.g., functional fragment, thereof.

In some embodiments, the Rep-coding region is operably linked to a first promoter and a second promoter, wherein (a) the first promoter is an baculovirus early-late promoter and the second promoter is a baculovirus very late promoter, (b) the first promoter is a baculovirus very late promoter and the second promoter is a baculovirus early-late promoter, (c) the first promoter is a baculovirus early promoter and the second promoter is a baculovirus early-late promoter, (d) the first promoter is a baculovirus early-late promoter and the second promoter is a baculovirus early promoter, (e) the first promoter is a baculovirus early promoter and the second promoter is a baculovirus late promoter, (f) the first promoter is a baculovirus late promoter and the second promoter is a baculovirus early promoter, (g) the first promoter is a baculovirus early-late promoter and the second promoter is a baculovirus late promoter, (h) the first promoter is a baculovirus late promoter and the second promoter is a baculovirus early-late promoter, (i) the first promoter is a baculovirus late promoter and the second promoter is a baculovirus very-late promoter, (j) the first promoter is a baculovirus very-late promoter and the second promoter is a baculovirus late promoter, (k) the first promoter is a baculovirus early promoter and the second promoter is a baculovirus very late promoter, (1) the first promoter is a baculovirus very late promoter and the second promoter is a baculovirus early promoter, (m) the first promoter is a baculovirus early promoter and the second promoter is a baculovirus early promoter, (n) the first promoter is a baculovirus early-late promoter and the second promoter is a baculovirus early-late promoter, or (o) the first promoter is a baculovirus late promoter and the second promoter is a baculovirus late promoter.

In some embodiments, the first promoter is a baculovirus early promoter or a baculovirus early-late promoter (e.g., a gp64 promoter). In some embodiments, the second promoter is a baculovirus late promoter or a baculovirus very late promoter (e.g., a polh promoter).

In some embodiments, the first or second promoter is a gp64 promoter (e.g., an OpMNPV gp64 promoter). In some embodiments, the gp64 promoter comprises the nucleotide sequence of SEQ ID NO: 217; a nucleotide sequence at least 70%, 75%, 80%, 85%, 90%, 95%, 96%, 97%, 98%, or 99% identical thereto; a nucleotide sequence comprising at least one, two, three, four, five, six, or seven, but no more than ten different nucleotides relative to SEQ ID NO: 217; or a nucleotide sequence comprising at least one, two, three, four, five, six, or seven, but no more than ten modifications (e.g., substitutions) relative to SEQ ID NO: 217.

In some embodiments, the first or second promoter is a polh promoter (e.g., an OpMNPV polh promoter or an AcMNPV polh promoter). In some embodiments, the polh promoter comprises the nucleotide sequence of SEQ ID NO: 167 or SEQ ID NO: 220; a nucleotide sequence at least 70%, 75%, 80%, 85%, 90%, 95%, 96%, 97%, 98%, or 99% identical thereto; a nucleotide sequence comprising at least one, two, three, four, five, six, or seven, but no more than ten different nucleotides relative to SEQ ID NO: 167 or SEQ ID NO: 220; or a nucleotide sequence comprising at least one, two, three, four, five, six, or seven, but no more than ten modifications (e.g., substitutions) relative to SEQ ID NO: 167 or SEQ ID NO: 220.

In some embodiments, the first promoter is a gp64 promoter and the second promoter is a polh promoter, or the first promoter is a polh promoter and the second promoter is a gp64 promoter. In some embodiments, the first promoter is a gp64 promoter and the second promoter is a polh promoter, wherein the combined promoters comprise the nucleotide sequence of SEQ ID NO: 221; a nucleotide sequence at least 70%, 75%, 80%, 85%, 90%, 95%, 96%, 97%, 98%, or 99% identical thereto; a nucleotide sequence comprising at least one, two, three, four, five, six, or seven, but no more than ten different nucleotides relative to SEQ ID NO: 221; or a nucleotide sequence comprising at least one, two, three, four, five, six, or seven, but no more than ten modifications (e.g., substitutions) relative to SEQ ID NO: 221.

In some embodiments, the first promoter is a baculovirus early-late promoter and the second promoter which is baculovirus very late promoter, e.g., a gp64 promoter and a polh promoter, optionally, wherein the Rep-coding region is present downstream of a homologous repeat region hr5.

Baculovirus early promoters, early-late promoters, late promoters, and very late promoters are described in detail, for example, in Chen et al. (Journal of Virology 2013; 87:6391-405, the contents of which are herein incorporated by reference in their entirety; see, in particular, Supplemental Table S2 of Chen et al.). In some embodiments, the promoter used in a baculovirus expression construct, variant baculovirus genome, or AAV expression construct described herein is a promoter provided in Chen et al. (Journal of Virology 2013; 87:6391-405, the contents of which are herein incorporated by reference in their entirety; see, in particular, Supplemental Table S2 of Chen et al.).

In some embodiments, a baculovirus early promoter is recognized by the host cell RNA polymerase II, and may contain a TATA box motif and/or a CATG motif. In some embodiments, transcription initiated from a baculovirus early promoter is alpha-amanatin resistant. Exemplary baculovirus early promoters include (but are not limited to) a lef3 promoter, a dbp promoter, a p35 promoter, an orf82 promoter, an get promoter, an orf81 promoter, an orf122 promoter, a pk-2 promoter, an orf55 promoter, an etl promoter, a hcf-1 promoter, an etm promoter, a lef-2 promoter, a lef-6 promoter, an orf84 promoter, an orf118 promoter, and an orf111 promoter.

In some embodiments, a baculovirus early-late promoter may contain components of both an early promoter and a late promoter. In some embodiments, an early-late promoter may comprise, e.g., a TATA box motif and/or a CATG motif and a TAAG motif (e.g., an ATAAG nucleotide sequence). Exemplary baculovirus early-late promoters include (but are not limited to) a lef2 promoter, a orf13 promoter, a orf23 promoter, a pkip promoter, a v-fgf promoter, a pp31 promoter, an odv-e66 promoter, an orf74 promoter, an orf79 promoter, an orf82 promoter, a p15 promoter, a cg30 promoter, a helicase promoter, an he65 promoter, an orf114 promoter, a pk-2 promoter, a gp64 promoter, a gp16 promoter, an alk-exo promoter, a p35 promoter, a me53 promoter, and an ie0 promoter. In some embodiments, the first or second promoter is an early-late promoter is a gp64 promoter. In some embodiments, the first or second promoter is an early-late promoter is a OpMNPV gp64 promoter. The gp64 promoter is described in, e.g., Garrity et al., Virology 1997; 231:167-81 and Blissard & Rohrmann. Virology 1989; 170:537-55, the contents of which are herein incorporated by reference in their entirety. In some embodiments, the first or second promoter is a p35 promoter, e.g., as described in Hershberger et al. J. Virology 1994; 68:3467-77 and Nissen & Friesen. J. Virology 1989; 63:493-503, the contents of each of which are herein incorporated by reference in their entirety. In some embodiments, the first or second promoter is a v-fgf promoter, e.g., as described in Detvisitsakun et al. Virology 2005; 336:308-17, the contents of which are herein incorporated by reference in their entirety. In some embodiments, the first or second promoter is a pp31 promoter, e.g., as described in Guarino & Summers. J. Virology 1986; 57:563-71, the contents of which are herein incorporated by reference in their entirety.

In some embodiments, a baculovirus late promoter is recognized by the baculovirus RNA polymerase. A late promoter may comprise, e.g., a TAAG motif (e.g., an ATAAG nucleotide sequence). Exemplary baculovirus late promoters include (but are not limited to) a ptpase promoter, an Ac-bro promoter, a ctx promoter, an orf5 promoter, an orf19 promoter, an orf20 promoter, an sod promoter, a HisP promoter, an orf34 promoter, a v-ubi promoter, an orf38 promoter, an orf43 promoter, an orf44 promoter, an orf56 promoter, an orf59 promoter, an orf60 promoter, and an fp-25k promoter.

In some embodiments, a baculovirus very late promoter is recognized by a viral (e.g., baculovirus) RNA polymerase and very late factor 1 (VLF-1). In some embodiments, a very late promoter may comprise, e.g., a TAAG motif (e.g., an ATAAG nucleotide sequence) and/or a proximal burst element sequence. In some embodiments, VLF-1 binds to the burst element sequence. Exemplary baculovirus late promoters include (but are not limited to) a p10 promoter and a polh promoter. In some embodiments, the first or second promoter is a polh promoter. In some embodiments, the first or second promoter is an OpMNPV polh promoter.

In some embodiments, the viral expression construct described herein comprises in 5′ to 3′ order: a first promoter (e.g., a baculovirus early-late promoter such as a gp64 promoter), a second promoter (e.g., a baculovirus very late promoter such as a polh promoter), and a Rep-coding region comprising a nucleotide sequence encoding a Rep78 protein and a Rep52 protein, e.g., a single polycistronic ORF encoding a Rep78 protein and a Rep52 protein.

In some embodiments, the Rep-coding region is present downstream of the homologous repeat region hr5. In some embodiments, the Rep-coding region is present in the p74 locus of the variant baculovirus genome, and is operably linked to a gp64 promoter and a polh promoter.

In some embodiments, the first promoter results in transcription of the Rep-coding region prior to transcription of the VP-coding region. In some embodiments, the first and second promoters result in transcription of the Rep-coding region prior to transcription of the VP-coding region.

In some embodiments, a baculovirus expression construct or variant baculovirus genome described herein comprises a nucleotide sequence encoding an assembly activation protein (AAP) protein. In some embodiments, the AAP protein is present in a location in the variant baculovirus genome chosen from ChiA, v-cath, p10, egt, polyhedrin, SOD, ctx, p26, odv-e56, p74 (PIF0), PIF1, PIF2, PIF3, PIF4, PIF5, Tn7, AcORF-91, AcORF-108, AcORF-52, v-ubi, or p94. In some embodiments, the AAP protein is present in the p10, p26, and/or p74 gene loci, optionally wherein p10, p26, and/or p74 genes were deleted. In some embodiments, the nucleotide sequence encoding the AAP protein is operably linked to a promoter, e.g., a gp64 promoter (e.g., a promoter comprising the nucleotide sequence of SEQ ID NO: 217, a nucleotide sequence at least 70%, 75%, 80%, 85%, 90%, 95%, 96%, 97%, 98%, or 99% identical thereto; a nucleotide sequence comprising at least one, two, three, four, five, six, or seven, but no more than ten different nucleotides relative to SEQ ID NO: 217; or a nucleotide sequence comprising at least one, two, three, four, five, six, or seven, but no more than ten modifications (e.g., substitutions) relative to SEQ ID NO: 217). In some embodiments, the encoded AAP protein is an AAV2 AAP protein. In some embodiments, the encoded AAP protein comprises the amino acid sequence of SEQ ID NO: 218; an amino acid sequence comprising at least 70%, 75%, 80%, 85%, 90%, 95%, 96%, 97%, 98%, 99% sequence identity to SEQ ID NO: 218; an amino acid sequence comprising at least one, two, three, four, five, six or seven modifications, e.g., substitutions (e.g., conservative substitutions), insertions, or deletions, but no more than 30, 20, or 10 modifications, e.g., substitutions (e.g., conservative substitutions), insertions, or deletions, relative to SEQ ID NO: 218; or an amino acid sequence comprising at least one, two, three, four, five, six or seven, but no more than 30, 20, or 10 different amino acids relative to SEQ ID NO: 218. In some embodiments, the nucleotide sequence encoding the AAP protein comprises the nucleotide sequence of SEQ ID NO: 219; a nucleotide sequence comprising at least 70%, 75%, 80%, 85%, 90%, 95%, 96%, 97%, 98%, 99% sequence identity to SEQ ID NO: 219; a nucleotide sequence comprising at least one, two, three, four, five, six or seven modifications, e.g., substitutions (e.g., conservative substitutions), insertions, or deletions, but no more than 30, 20, or 10 modifications, e.g., substitutions (e.g., conservative substitutions), insertions, or deletions, relative to SEQ ID NO: 219; or a nucleotide sequence comprising at least one, two, three, four, five, six or seven, but no more than 30, 20, or 10 different nucleotides relative to SEQ ID NO: 219.

TABLE 5
Exemplary AAP protein sequences

| | SEQ | |
|---|---|---|
| | ID | |
| Description | NO: | Sequences |
| AAP (amino | 218 | METQTQYLTPSLSDSHQQPPLVWELIRW |
| acid) | | LQAVAHQWQTITRAPTEWVIPREIGIAI |
| | | PHGWATESSPPAPEPGPCPPTTTTSINK |
| | | FPANQEPRTTITTLATAPLGGILTSTDS |
| | | TATFHHVTGKDSSITTGDSDPRDSTSSS |
| | | LIFKSKRSRRMTVRRRLPITLPARFRCL |
| | | LTRSTSSRTSSARRIKDASRRSQQTSSW |
| | | CHSMDTSP |
| AAP (DNA) | 219 | ATGGAGACACAGACGCAGTACCTGACGC |
| | | CATCATTGTCTGATAGCCATCAGCAGCC |
| | | TCCGCTTGTATGGGAACTTATACGTTGG |
| | | CTCCAGGCCGTCGCCCATCAATGGCAGA |
| | | CGATCACTAGGGCTCCTACCGAATGGGT |
| | | AATCCCCAGAGAGATTGGTATCGCCATC |
| | | CCCCATGGCTGGGCTACCGAATCATCTC |
| | | CTCCCGCCCCTGAGCCAGGTCCTTGTCC |
| | | TCCGACAACTACAACTTCGACGAATAAA |
| | | TTCCCGGCTAACCAAGAGCCACGTACTA |
| | | CCATCACAACTTTGGCAACAGCTCCACT |
| | | GGGTGGAATACTCACAAGCACAGACTCG |
| | | ACTGCTACGTTCCACCATGTGACAGGTA |
| | | AGGATAGTAGTACAACGACCGGCGACTC |
| | | TGATCCAAGGGACTCTACGTCATCGTCT |
| | | TTGACTTTTAAGTCGAAAAGAAGCCGTC |
| | | GTATGACTGTTCGTCGCAGGCTTCCCAT |
| | | TACCCTTCCCGCTAGGTTCAGATGCCTC |
| | | CTTACACGCAGTACCAGTTCCAGAACTT |
| | | CATCGGCTAGGAGAATAAAGGATGCTTC |
| | | TAGACGCAGCCAACAAACTAGTTCATGG |
| | | TGCCATTCCATGGACACTAGCCCAAGCG |
| | | GAGACTATAAGGATGATGACGACAAGTA |
| | | ATAA |

In some embodiments, a baculovirus expression construct or a baculovirus variant genome described herein comprises a minicistron. In some embodiments, the minicistron sequence is from a baculovirus gene; optionally a baculovirus gp64 gene. In some embodiments, minicistron sequence comprises SEQ ID NO: 9 or SEQ ID NO: 10; a nucleotide sequence at least 70%, 75%, 80%, 85%, 90%, 95%, 96%, 97%, 98%, or 99% identical to SEQ ID NO: 9 or 10; a nucleotide sequence comprising one, two, or three modifications (e.g., substitutions), but no more than four modifications (e.g., substitutions) relative to SEQ ID NO: 9 or 10; or a nucleotide sequence comprising one, two, or three, but no more than four different nucleotides relative to SEQ ID NO: 9 or 10.

TABLE 6
Exemplary minicistron sequences

| | SEQ | |
|---|---|---|
| | ID | |
| Description | NO: | Sequences |
| Minicistron variant 1 | 9 | ATGAACAAAGATTAA |
| Minicistron variant 2 | 10 | ATGCCTCAATGCTACTAG |

In some embodiments, a baculovirus expression construct or a baculovirus variant genome described herein comprises a modified Kozak sequence. In some embodiments, the modified Kozak sequence is present upstream of a VP coding region, e.g., a VP1, a VP2, or a VP3 coding region. In some embodiments, the modified Kozak sequence is present upstream of a Rep coding region, e.g., a Rep52 coding region or a Rep78 coding region. In some embodiments, the modified Kozak sequence comprises a sequence as provided in Table 7. In some embodiments, the modified Kozak sequence comprises a sequence as provided in Table 8. In some embodiments, the modified Kozak sequence comprises a sequence as described in Noderer, William L., et al. “Quantitative analysis of mammalian translation initiation sites by FACS-seq.” Molecular systems biology 10.8 (2014): 748; and Diaz de Arce et al. “Complete motif analysis of sequence requirements for translation initiation at non-AUG start codons” Nucleic Acids Res. 2018 January 46(2):985-994; and Kondratov et. al. “Direct Head-to-Head Evaluation of Recombinant Adeno-associated Viral Vectors Manufactured in Human versus Insect Cells” Molecular Therapy. 2017 Dec. 25(12):2661-2675, the contents of which are hereby incorporated by reference in their entirety. In some embodiments, the modified Kozak sequence comprises a sequence provided in US20200123572, WO2017181162, and WO2021222472, the contents of which are hereby incorporated by reference in their entirety.

TABLE 7
Exemplary Modified Kozak Sequences

| | | SEQ | | SEQ |
|---|---|---|---|---|
| | Sequence | ID | Sequence | ID |
| Name | (RNA) | NO: | (DNA) | NO: |
| VP1aug13 | cgguguAUGgc | 21 | cggtgtATGgc | 32 |
| VP1aug14 | cgguguAUGag | 22 | cggtgtATGag | 33 |
| VP1aug28 | uaguggAUGag | 23 | tagtggATGag | 34 |
| VP1aug29 | uauuggAUGag | 24 | tattggATGag | 35 |
| VP1aug30 | caguggAUGag | 25 | cagtggATGag | 36 |
| VP1aug31 | cauuggAUGag | 26 | cattggATGag | 37 |
| VP1aug32 | ugguggAUGag | 27 | tggtggATGag | 38 |
| VP1aug33 | aauuggAUGag | 28 | aattggATGag | 39 |
| VP1aug34 | aaguggAUGag | 29 | aagtggATGag | 40 |
| VP1aug35 | ccguggAUGag | 30 | ccgtggATGag | 41 |
| VP1aug36 | ggguggAUGag | 31 | gggtggATGag | 42 |
| VP1aug52 | agguguAUGgc | 222 | aggtgtATGgc | 223 |

TABLE 8
Exemplary Modified Kozak Sequences

| | SEQ |
|---|---|
| | ID |
| | NO: |
| Sequence (RNA) | |
| UAGUGUAUGUC | 73 |
| UAGUUUAUGUC | 74 |
| UGUUUUAUGUC | 75 |
| UCUUUUAUGUC | 76 |
| UAGUGUAUGGG | 77 |
| UAGUUUAUGGG | 78 |
| UGUUUUAUGGG | 79 |
| UCUUUUAUGGG | 80 |
| UAGUGUAUGGC | 81 |
| UAGUUUAUGGC | 82 |
| UGUUUUAUGGC | 83 |
| UCUUUUAUGGC | 84 |
| CAUUGUAUGUC | 85 |
| UCGUUUAUGGA | 86 |
| CAGUUUAUGGU | 87 |
| CAUUGUAUGGU | 88 |
| UAGUGUAUGCU | 89 |
| CAUUGUAUGCU | 90 |
| UCUUUUAUGUC | 91 |
| UGUUUUAUGUC | 92 |
| UAGUUUAUGUC | 93 |
| UAGUGUAUGUC | 94 |
| CGACAUGAAUGUC | 95 |
| AGGGAUGGAUGUC | 96 |
| CUUGAUGGAUGUC | 97 |
| CGUGAUGUAUGUC | 98 |
| GGCGAUGGAUGUC | 99 |
| CGUGAUGGAUGUC | 100 |
| CGUAAUGCAUGUC | 101 |
| GAACAUGAAUGUC | 102 |
| GGACAUGCAUGUC | 103 |
| AAGAAUGAAUGUC | 104 |
| CCCGAUGGAUGUC | 105 |
| GCCGAUGUAUGUC | 106 |
| CGGGAUGAAUGUC | 107 |
| GACCAUGCAUGGCU | 108 |
| AAUUAUGCAUGGC | 109 |
| AGACAUGUAUGGC | 110 |
| AAGAAUGUAUGGC | 111 |
| GACCAUGCAUGGCU | 112 |
| CGCGAUGGAUGGC | 113 |
| AGCGAUGGAUGGC | 114 |
| GAUCAUGCAUGGC | 115 |
| CGUAAUGGAUGGC | 116 |
| AACGAUGUAUGGC | 117 |
| Sequence (DNA) | |
| TAGTGTATGTC | 118 |
| TAGTTTATGTC | 119 |
| TGTTTTATGTC | 120 |
| TCTTTTATGTC | 121 |
| TAGTGTATGGG | 122 |
| TAGTTTATGGG | 123 |
| TGTTTTATGGG | 124 |
| TCTTTTATGGG | 125 |
| TAGTGTATGGC | 126 |
| TAGTTTATGGC | 127 |
| TGTTTTATGGC | 128 |
| TCTTTTATGGC | 129 |
| CATTGTATGTC | 130 |
| TCGTTTATGGA | 131 |
| CAGTTTATGGT | 132 |
| CATTGTATGGT | 133 |
| TAGTGTATGCT | 134 |
| CATTGTATGCT | 135 |
| TCTTTTATGTC | 136 |
| TGTTTTATGTC | 137 |
| TAGTTTATGTC | 138 |
| TAGTGTATGTC | 139 |
| CGACATGAATGTC | 140 |
| AGGGATGGATGTC | 141 |
| CTTGATGGATGTC | 142 |
| CGTGATGTATGTC | 143 |
| GGCGATGGATGTC | 144 |
| CGTGATGGATGTC | 145 |
| CGTAATGCATGTC | 146 |
| GAACATGAATGTC | 147 |
| GGACATGCATGTC | 148 |
| AAGAATGAATGTC | 149 |
| CCCGATGGATGTC | 150 |
| GCCGATGTATGTC | 151 |
| CGGGATGAATGTC | 152 |
| GACCATGCATGGCT | 153 |
| AATTATGCATGGC | 154 |
| AGACATGTATGGC | 155 |
| AAGAATGTATGGC | 156 |
| GACCATGCATGGCT | 157 |
| CGCGATGGATGGC | 158 |
| AGCGATGGATGGC | 159 |
| GATCATGCATGGC | 160 |
| CGTAATGGATGGC | 161 |
| AACGATGTATGGC | 162 |

In some embodiments, the baculovirus expression construct comprises one or more (e.g., 1, 2, 3, 4, or 5 or more) polynucleotide inserts (e.g., a Rep-coding region and/or a VP-coding region) which are inserted into the locus of one or more non-essential baculovirus genes. In some embodiments, the non-essential baculovirus genes (e.g., auxiliary and/or per os infectivity factor genes) are chosen from egt, p74 (PIF0), p26, SOD, ChiA, v-cath, p10, polyhedrin, ctx, odv-e56, PIF1, PIF2, PIF3, PIF4, PIF5, Tn7, AcORF-91, AcORF-108, AcORF-52, v-ubi, or p94.

In some embodiments, the baculovirus expression construct comprises at least a portion of a baculovirus genome, e.g., a variant baculovirus genome, comprising a disruption of at least two non-essential genes (e.g., auxiliary and/or per os infectivity factor genes), wherein the at least two non-essential genes are independently chosen from egt, p74 (PIF0), p26, SOD, ChiA, v-cath, p10, polyhedrin, ctx, odv-e56, PIF1, PIF2, PIF3, PIF4, PIF5, Tn7, AcORF-91, AcORF-108, AcORF-52, v-ubi, or p94. In some embodiments, the at least two non-essential genes comprise (i) (i) v-cath and egt; (ii) v-cath, egt, and SOD; (iii) chiA, v-cath, egt, p26, p10, and p74; (iv) chiA, v-cath, egt, p26, p10, p74, and SOD; or (v) chiA, v-cath, egt, p26, p10, p74, SOD, AcORF-91, and AcORF-108; or (vi) chiA, v-cath, egt, p26, p10, p74, and SOD. In some embodiments, the disruption comprises a deletion of a chiA gene, a v-cath gene, a p26 gene, a p10 gene, a p74 gene, and/or a SOD gene, or a portion thereof. In some embodiments, one of the at least two non-essential genes is the p74 gene which is present downstream of a homologous repeat region 5 (hr5).

In some embodiments, the baculovirus expression construct or variant baculovirus genome comprises a Rep-coding region which is present in (e.g., and disrupts) the p74 locus of a variant baculovirus genome. In some embodiments, the baculovirus expression construct or variant baculovirus genome comprises an AAV expression construct. In some embodiments, the AAV expression construct comprises a VP-coding region which is present in the SOD locus of a variant baculovirus genome. In some embodiments, the AAV expression construct comprises a payload coding region which is present in (e.g., and disrupts) the v-cath locus of a variant baculovirus genome. In some embodiments, the AAV expression construct comprises a Rep-coding region which is present in (e.g., and disrupts) the p74 locus of a variant baculovirus genome and a VP coding region which is present in the SOD locus of the variant baculovirus genome. In some embodiments, the AAV expression construct comprises a Rep-coding region which is present in (e.g., and disrupts) the p74 locus of a variant baculovirus genome, a VP-coding region which is present in (e.g., and disrupts) the SOD locus of the variant baculovirus genome, and a payload coding region which is present in (e.g., and disrupts) the v-cath locus of the variant baculovirus genome.

In some embodiments, the baculovirus expression construct or variant baculovirus genome comprises (i) a Rep-coding region which is present in the p74 locus of the variant baculovirus genome, optionally wherein the Rep-coding region (e.g., a Rep-coding region encoding a Rep78 protein and a Rep52 protein, wherein the nucleotide sequence encoding the Rep52 protein is comprised within the nucleotide sequence encoding the Rep78 protein) is operably linked to a gp64 promoter and a polh promoter; (ii) a VP-coding region (e.g., a VP-coding region wherein the nucleotide sequence encoding the VP2 protein and the nucleotide sequence encoding the VP3 protein are comprised within the nucleotide sequence encoding the VP1 protein) which is present in the SOD locus of the variant baculovirus genome, optionally wherein the VP-coding region is operably linked to a p10 promoter; and (iii) a payload coding region which is present in the v-cath locus of the variant baculovirus and comprises a nucleotide sequence encoding a payload.

### Baculovirus Production Systems

In some embodiments, AAV particles or viral vectors are produced in a baculoviral system using a baculovirus expression construct and a payload construct. In some embodiments, the baculoviral system comprises a baculovirus expression construct (e.g., a bacmid) and/or baculovirus infected insect cells (BIICs). In some embodiments, the baculovirus expression construct and a payload construct may be, e.g., a bacmid, a baculovirus expression construct, or BAC described herein. Transfection of separate viral replication cell populations produces two or more groups (e.g., two, three) of baculoviruses, one or more group which can comprise the baculovirus expression construct (e.g., the baculovirus is an “Expression BEV” or “expressionBac”), and one or more group which can comprise the payload construct (e.g., the baculovirus is a “Payload BEV” or “payloadBac”). The baculoviruses may be used to infect a viral production cell for production of AAV particles or viral vector.

In some embodiments, the process comprises transfection of a single viral replication cell population to produce a single baculovirus (BEV) group which comprises both the baculovirus expression construct described herein and a payload construct described herein. These baculoviruses may be used to infect a viral production cell for production of AAV particles or viral vector.

In some embodiments, BEVs are produced using a Bacmid Transfection agent, such as Promega FuGENE HD, WFI water, or ThermoFisher Cellfectin II Reagent. In some embodiments, BEVs are produced and expanded in viral production cells, such as an insect cell.

In some embodiments, the method utilizes seed cultures of viral production cells that comprise one or more BEVs, comprising baculovirus infected insect cells (BIICs). The seed BIICs have been transfected/transduced/infected with an Expression BEV which comprises a baculovirus expression construct described herein, and also a Payload BEV which comprises a payload construct described herein. In some embodiments, the seed cultures are harvested, divided into aliquots and frozen, and may be used at a later time to initiate transfection/transduction/infection of a naïve population of production cells.

Baculovirus expression vectors (BEV) for producing AAV particles in insect cells, comprising but not limited to Spodopterafrugiperda (Sf9) cells, provide high titers of viral vector product. Recombinant baculovirus encoding the baculovirus expression construct described herein and payload construct described herein initiates a productive infection of viral vector replicating cells. Infectious baculovirus particles released from the primary infection secondarily infect additional cells in the culture, exponentially infecting the entire cell culture population in a number of infection cycles that is a function of the initial multiplicity of infection, see Urabe, M. et al. J Virol. 2006 February;80(4):1874-85, the content of which is incorporated herein by reference in its entirety as related to the production and use of BEVs and viral particles, insofar as it does not conflict with the present disclosure.

In some embodiments, the production system addresses baculovirus instability over multiple passages by utilizing a titerless infected-cells preservation and scale-up system. Small scale seed cultures of viral producing cells are transfected with baculovirus expression constructs encoding the structural and/or non-structural components of the AAV particles. Baculovirus-infected viral producing cells are harvested into aliquots that may be cryopreserved in liquid nitrogen; the aliquots retain viability and infectivity for infection of large scale viral producing cell culture Wasilko D J et al. Protein Expr Purif. 2009 June;65(2):122-32, the content of which is incorporated herein by reference in its entirety as related to the production and use of BEVs and viral particles, insofar as it does not conflict with the present disclosure.

A genetically stable baculovirus may be used to produce a source of the one or more of the components for producing AAV particles in invertebrate cells. In some embodiments, defective baculovirus expression constructs may be maintained episomally in insect cells. In such an embodiment, the corresponding baculovirus expression construct is engineered with replication control elements, comprising but not limited to promoters, enhancers, and/or cell-cycle regulated replication elements.

In some embodiments, baculoviruses may be engineered with a marker for recombination into the chitinase/cathepsin locus. The chia/v-cath locus is non-essential for propagating baculovirus in tissue culture, and the V-cath (EC 3.4.22.50) is a cysteine endoprotease that is most active on Arg-Arg dipeptide containing substrates. The Arg-Arg dipeptide is present in densovirus and parvovirus capsid structural proteins but infrequently occurs in dependovirus VP1.

In some embodiments, stable viral producing cells permissive for baculovirus infection are engineered with at least one stable integrated copy of any of the elements necessary for AAV replication and vector production comprising, but not limited to, the entire AAV genome, Rep and Cap genes, Rep genes, Cap genes, each Rep protein as a separate transcription cassette, each VP protein as a separate transcription cassette, the AAP (assembly activation protein), or at least one of the baculovirus helper genes with native or non-native promoters.

In some embodiments, baculoviral inoculum banks can be produced using small-scale shake flasks, such as 3 L or 5 L shake flasks. In some embodiments, baculoviral inoculum banks can be produced using bioreactors, such as 20-50 L bioreactors.

In some embodiments, perfusion technology can be used in the production of baculoviral inoculum banks. Perfusion systems are fluid circulation systems which use combinations of pumps, filters, and screens to retain cells inside a bioreactor while continually removing cell waste products and replacing media depleted of nutrients by cell metabolism. In some embodiments, the perfusion system is an alternating tangential flow (ATF) perfusion system. In some embodiments, a perfusion system can be used in coordination with bioreactors to manage and cycle cell culture media within a bioreactor during the production of Baculovirus Infected Insect Cells (BIICs). In some embodiments, a perfusion system can be used to support the production of high quality BIIC banks having an unexpectedly high cell density at large-scale. In some embodiments, a perfusion system can be used to provide an infection-cell-to-product-cell yield of greater than 70% (e.g., 75-80%, 80-85%, 85-90%, 90-95% or 95-100%). In some embodiments, a perfusion system can be used to perform a media switch within the bioreactor, such as the replacements of a cell culture media with a cryopreservation media which allows for BIIC cells to be frozen and preserved.

Also provided herein are methods for producing a baculovirus infected insect cell (BIIC), e.g., expression BIICs and/or payload BIICs. In some embodiments, the method comprises the following steps: (a) introducing a volume of cell culture medium into a bioreactor; (b) introducing at least one viral production cell (VPC) into the bioreactor and expanding the number of VPCs in the bioreactor to a target VPC cell density; (c) introduction at least one Baculoviral Expression Vector (BEV) into the bioreactor, wherein the BEV comprises an AAV viral expression construct or an AAV payload construct; (d) incubating the mixture of VPCs and BEVs in the bioreactor under conditions which allow at least one BEV to infect at least one VPC to produce a baculovirus infected insect cell (BIIC); (e) incubating the bioreactor under conditions which allow the number of BIICs in the bioreactor to reach a target BIIC cell density; and (f) harvesting the BIICs from the bioreactor. In some embodiments, the bioreactor has a volume of at least 5 L, 10 L, 20 L, 50 L, 100 L, or 200 L. In some embodiments, the volume of cell culture medium (i.e., working volume) in the bioreactor is at least 5 L, 10 L, 20 L, 50 L, 100 L, or 200 L.

In some embodiments, provided herein is a method of producing an adeno-associated virus (AAV) comprising a polynucleotide encoding a payload, the method comprising:
(a) culturing VPCs in a bioreactor to a target cell density, wherein the bioreactor comprises a cell culture medium described herein,
(b) introducing into the bioreactor at least one baculovirus (expressionBac) comprising a baculovirus expression construct described herein, and at least one baculovirus (payloadBac) comprising a payload construct described herein, wherein the baculovirus expression construct comprises an adeno-associated virus (AAV) viral expression construct, and wherein the payload construct comprises the polynucleotide encoding the payload, and
(c) incubating the VPCs in the bioreactor under conditions that result in the production of one or more AAVs within one or more VPCs, wherein one or more of the AAVs comprise the polynucleotide encoding the payload. In some embodiments, the method further comprises a step of harvesting a viral production pool from the bioreactor, wherein the viral production pool comprises one or more VPCs comprising one or more AAVs.

In some embodiments, provided herein is a method for producing an adeno-associated virus (AAV) comprising a polynucleotide encoding a payload, the method comprising:
(a) providing viral production cells (VPCs) comprising at least one baculovirus (expressionBac) comprising an AAV viral expression construct, and at least one baculovirus (payloadBac) comprising a polynucleotide encoding the payload; and
(b) culturing the VPCs in a cell culture medium described herein, under conditions suitable for the production of an AAV comprising the payload; thereby producing the AAV comprising the payload. In some embodiments, prior to step (a), the at least one baculovirus (expressionBac) comprising an AAV viral expression construct, and the at least one baculovirus (payloadBac) comprising a polynucleotide encoding the payload into the VPCs, are introduced. In some embodiments, the method comprises a further step (c) of harvesting the AAV.

In some embodiments, the same baculovirus comprises the AAV viral expression construct, and the polynucleotide encoding the payload. In some embodiments, different baculoviruses comprises the AAV viral expression construct, and the polynucleotide encoding the payload.

In some embodiments, the at least one expressionBac is comprised in at least one VPC, e.g., an insect cell (expressionBIIC) and/or the at least one payloadBac is comprised in at least one VPC, e.g., insect cell (payloadBlIC).

In some embodiments, the VPCs are insect cells. In some embodiments, the insect cells are Sf9 cells.

In some embodiments, provided herein is an AAV produced by the methods described herein.

In some embodiments, the bioreactor can comprise a perfusion system for managing the cell culture medium within the bioreactor. In some embodiments, the perfusion system is an alternating tangential flow (ATF) perfusion system.

In some embodiments, BIICs (expression BIICs, payload BIICs) are used to transfect viral production cells, e.g., Sf9 cells. In some embodiments, baculoviruses comprising bacmids are used to transfect viral production cells, e.g., Sf9 cells.

### Therapeutic Applications

The present disclosure provides a method for treating a disease, disorder and/or condition in a subject, including a human subject, comprising administering to the subject an AAV particle described herein, e.g., an AAV particle comprising an AAV capsid polypeptide, or administering to the subject any of the described compositions, including a pharmaceutical composition, described herein.

In some embodiments, the AAV particle described herein is administered to a subject prophylactically, to prevent onset of disease. In another embodiment, the AAV particle (e.g., an AAV described herein is administered to treat (e.g., lessen the effects of) a disease or symptoms thereof. In yet another embodiment, the AAV particle described herein is administered to cure (eliminate) a disease. In another embodiment, the AAV particle described herein is administered to prevent or slow progression of disease. In yet another embodiment, the AAV particle described herein is used to reverse the deleterious effects of a disease. Disease status and/or progression may be determined or monitored by standard methods known in the art.

In some embodiments, the AAV particle described herein is useful for treatment, prophylaxis, palliation or amelioration of a genetic disorder, e.g., an autosomal dominant genetic disorder, an autosomal recessive disorder, X-linked dominant genetic disorder, an X-linked recessive genetic disorder, or a Y-linked genetic disorder. In some embodiments, the genetic disorder is a monogenetic disorder or a polygenic disorder. In some embodiments, treatment of a genetic disorder, e.g., a monogenic disorder, comprises the use of an AAV particle described herein for a gene replacement therapy.

In some embodiments, provided herein is method for treating a neurological disorder and/or neurodegenerative disorder in a subject, comprising administering to the subject an effective amount of a pharmaceutical composition described herein or an AAV particle, e.g., a plurality of particles, comprising an AAV capsid polypeptide described herein. In some embodiments, treatment of a neurological disorder and/or neurodegenerative disorder comprises prevention of said neurological disorder and/or neurological disorder.

In some embodiments, the AAV particle described herein is useful for the treatment, prophylaxis, palliation or amelioration of neurological diseases and/or disorders. In some embodiments, the AAV particle described herein is useful for the treatment, prophylaxis, palliation or amelioration of tauopathy.

In some embodiments, the AAV particle described herein is for the treatment, prophylaxis, palliation or amelioration of Alzheimer's Disease. In some embodiments, treatment of Alzheimer's Disease comprises the use of an AAV particle described herein for a gene replacement therapy. In some embodiments, the payload encoded by an AAV particle comprising a capsid polypeptide described herein comprises an ApoE2 protein, ApoE4 protein, an ApoE3 protein, BDNF protein, CYP46A1 protein, Klotho protein, fractalkine (FKN) protein, neprilysin protein (NEP), CD74 protein, caveolin-1, or a combination or variant thereof. In some embodiments, treatment of Alzheimer's Disease comprises the use of an AAV particle described for a reduction in the expression of a tau gene and/or protein, a synuclein gene and/or protein, or a combination or variant thereof. In some embodiments, the payload encoded by an AAV particle comprising a capsid polypeptide described herein comprises an antibody that binds to tau or synuclein, an RNAi agent for inhibiting tau or synuclein, a gene editing system (e.g., a CRISPR-Cas system) for altering tau or synuclein expression, or a combination thereof.

In some embodiments, the AAV particle described herein is for the treatment, prophylaxis, palliation or amelioration of frontal temporal dementia. In some embodiments, treatment of frontal temporal dementia comprises the use of an AAV particle described herein for a gene replacement therapy. In some embodiments, the payload encoded by an AAV particle comprising a capsid polypeptide described herein comprises a progranulin protein or variant thereof.

In some embodiments, the AAV particle described herein is useful the treatment, prophylaxis, palliation or amelioration of Friedreich's ataxia, or any disease stemming from a loss or partial loss of frataxin protein. In some embodiments, treatment of Friedreich's ataxia comprises the use of an AAV particle described herein for a gene replacement therapy.

In some embodiments, the AAV particle described herein is useful for the treatment, prophylaxis, palliation or amelioration of Parkinson's Disease. In some embodiments, treatment of Parkinson's disease comprises the use of an AAV particle described herein for a gene replacement therapy. In some embodiments, the payload encoded by an AAV particle comprising a capsid variant described herein comprises an AADC protein, GAD protein, GDNF protein, TH-GCH1 protein, GBA protein, AIMP2-DX2 protein, or a combination or variant thereof. In some embodiments, treatment of Parkinson's disease comprises the use of an AAV particle described herein for a gene knock-down therapy or a gene editing therapy (e.g., knock-out, repression, or correction). In some embodiments, the payload encoded by an AAV particle comprising a capsid variant described herein comprises a modulator, e.g., an RNAi agent or a CRISPR-Cas system, for altering expression of an alpha-synuclein gene, mRNA, and/or protein, or variant thereof.

In some embodiments, the AAV particle described herein is useful for the treatment, prophylaxis, palliation or amelioration of an AADC deficiency. In some embodiments, treatment of AADC deficiency comprises the use of an AAV particle described herein for a gene replacement therapy. In some embodiments, the payload encoded by an AAV particle comprising a capsid variant described herein comprises an AADC protein or variant thereof.

In some embodiments, the AAV particle described herein is useful for the treatment, prophylaxis, palliation or amelioration of Amyotrophic lateral sclerosis (ALS). In some embodiments, treatment of ALS comprises the use of an AAV particle described herein for a gene replacement therapy. In some embodiments, the payload encoded by an AAV particle comprising a capsid variant described herein comprises an TDP-43 protein, UPF1 protein, C9orf72 protein, CCNF protein, HSF1 protein, Factor H protein, NGF protein, ADAR2 protein, GDNF protein, VEGF protein, HGF protein, NRTN protein, AIMP2-DX2 protein, or a combination or variant thereof. In some embodiments, treatment of ALS comprises the use of an AAV particle described herein for a gene knock-down therapy or a gene editing therapy (e.g., knock-out, repression, or correction). In some embodiments, the payload encoded by an AAV particle comprising a capsid polypeptide described herein comprises a modulator, e.g., an RNAi agent or a CRISPR-Cas system, for altering expression of a SOD1 or C90RF72 gene, mRNA, and/or protein, or a combination or variant thereof.

In some embodiments, the AAV particle described herein is useful for the treatment, prophylaxis, palliation or amelioration of Huntington's Disease. In some embodiments, treatment of ALS comprises the use of an AAV particle described herein for a gene knock-down (e.g., knock-out) therapy or a gene editing therapy (e.g., knock-out, repression, or correction). In some embodiments, the payload encoded by an AAV particle comprising a capsid variant described herein comprises a modulator, e.g., an RNAi agent or a CRISPR-Cas system, for altering expression of an HTT gene, mRNA, and/or protein, or a variant thereof.

In some embodiments, the AAV particle described herein is useful for the treatment, prophylaxis, palliation or amelioration of spinal muscular atrophy. In some embodiments, treatment of spinal muscular atrophy comprises the use of an AAV particle described herein for a gene replacement therapy. In some embodiments, the payload encoded by an AAV particle comprising a capsid variant described herein comprises an SMN1 protein, an SMN2 protein, or a combination or variant thereof.

In some embodiments, the AAV particle described herein is useful for the treatment, prophylaxis, palliation or amelioration of multiple system atrophy. In some embodiments, treatment of multiple system atrophy comprises the use of an AAV particle described herein for a gene replacement therapy.

In some embodiments, the AAV particle described herein is useful for the treatment, prophylaxis, palliation or amelioration of Gaucher disease (GD) (e.g., Type 1 GD, Type 2 GD, or Type 3 GD). In some embodiments, the AAV particle described herein is useful for the treatment, prophylaxis, palliation or amelioration of Parkinson's disease associated with a GBA mutation. In some embodiments, the AAV particle described herein is useful for the treatment, prophylaxis, palliation or amelioration of dementia with Lewy Bodies (DLB).

In some embodiments, the AAV particle described herein is useful for treatment, prophylaxis, palliation or amelioration of a leukodystrophy, e.g., Alexander disease, autosomal dominant leukodystrophy with autonomic diseases (ADLD), adrenoleukodystrophy (ALD),Canavan disease, cerebrotendinous xanthomatosis (CTX), metachromatic leukodystrophy (MLD), Pelizaeus-Merzbacher disease, or Refsum disease. In some embodiments, treatment of MLD comprises the use of an AAV particle described herein for a gene replacement therapy. In some embodiments, the payload encoded by an AAV particle comprising a capsid variant described herein comprises an ARSA protein or variant thereof. In some embodiments, treatment of ALD comprises the use of an AAV particle described herein for a gene replacement therapy. In some embodiments, the payload encoded by an AAV particle comprising a capsid polypeptide described herein comprises an ABCD-1 protein or variant thereof.

In some embodiments, the AAV particle described herein is useful for the treatment, prophylaxis, palliation, or amelioration of megalencephalic leukoencephalopathy (MLC). In some embodiments, treatment of MLC comprises the use of an AAV particle described herein for a gene replacement therapy. In some embodiments, the payload encoded by an AAV particle comprising a capsid polypeptide described herein comprises an MLC1 protein or variant thereof.

In some embodiments, the AAV particle described herein is useful for the treatment, prophylaxis, palliation, or amelioration of Krabbe disease. In some embodiments, treatment of Krabbe disease comprises the use of an AAV particle described herein for a gene replacement therapy. In some embodiments, the payload encoded by an AAV particle comprising a capsid polypeptide described herein comprises a GALC protein or variant thereof.

In some embodiments, the AAV particle described herein is useful for the treatment, prophylaxis, palliation, or amelioration of Mucopolysaccharidosis, e.g., a Type I (MPS I), Type II (MPS II), Type IIIA (MPS IIIA), Type IIIB (MPS IIIB), or Type IIIC (MPS IIIC). In some embodiments, treatment of Mucopolysaccharidosis comprises the use of an AAV particle described herein for a gene replacement therapy or a gene editing therapy (e.g., enhancement or correction). In some embodiments, the payload encoded or corrected by an AAV particle comprising a capsid polypeptide described herein comprises an IDUA protein, IDS protein, SGSH protein, NAGLU protein, HGSNAT protein, or a combination or variant thereof.

In some embodiments, the AAV particle described herein is useful for the treatment, prophylaxis, palliation, or amelioration of Batten/NCL. In some embodiments, treatment of Batten/NCL comprises the use of an AAV particle described herein for a gene replacement therapy. In some embodiments, the payload encoded by an AAV particle described herein comprises a CLN1 protein, CLN2 protein, CLN3 protein, CLN5 protein, CLN6 protein, CLN7 protein, CLN8 protein, or a combination or variant thereof.

In some embodiments, the AAV particle described herein is useful for the treatment, prophylaxis, palliation or amelioration of Rett Syndrome. In some embodiments, treatment of Rett Syndrome comprises the use of an AAV particle described herein (e.g., an AAV particle comprising an AAV capsid polypeptide described herein) for a gene replacement therapy. In some embodiments, the payload encoded by an AAV particle comprising a capsid polypeptide described herein comprises an MeCP2 protein or variant thereof.

In some embodiments, the AAV particle described herein is useful for the treatment, prophylaxis, palliation, or amelioration of Angelman Syndrome. In some embodiments, treatment of Angelman Syndrome comprises the use of an AAV particle described herein for a gene replacement therapy. In some embodiments, the payload encoded by an AAV particle comprising a capsid polypeptide described herein comprises a UBE3A protein or variant thereof.

In some embodiments, the AAV particle described herein is useful for the treatment, prophylaxis, palliation, or amelioration of Fragile X Syndrome. In some embodiments, treatment of Fragile X Syndrome comprises the use of an AAV particle described herein for a gene replacement therapy. In some embodiments, the payload encoded by an AAV particle comprising a capsid polypeptide described herein comprises a Reelin protein, a DgkK protein, a FMR1 protein, or a combination or variant thereof.

In some embodiments, the AAV particle described herein is useful for the treatment, prophylaxis, palliation, or amelioration of Canavan Disease. In some embodiments, treatment of Canavan Disease comprises the use of an AAV particle described herein for a gene replacement therapy. In some embodiments, the payload encoded by an AAV particle comprising a capsid polypeptide described herein comprises an ASPA protein or variant thereof.

In some embodiments, the AAV particle described herein is useful for the treatment, prophylaxis, palliation, or amelioration of a Gangliosidosis, e.g., a GM1 Gangliosidosis or a GM2 Gangliosidosis (e.g., Tay Sachs Sandhoff). In some embodiments, treatment of a Gangliosidosis, e.g., a GM1 Gangliosidosis or a GM2 Gangliosidosis (e.g., Tay Sachs Sandhoff), comprises the use of an AAV particle described herein for a gene replacement therapy. In some embodiments, the payload encoded by an AAV particle comprising a capsid polypeptide described herein comprises a GLB1 protein, a HEXA protein, a HEXB protein, a GM2A protein, or a combination or variant thereof.

In some embodiments, the AAV particle described herein is useful for the treatment, prophylaxis, palliation, or amelioration of GM3 Synthase Deficiency. In some embodiments, treatment of GM3 Synthase Deficiency comprises the use of an AAV particle described herein for a gene replacement therapy. In some embodiments, the payload encoded by an AAV particle comprising a capsid polypeptide described herein comprises an ST3GAL5 protein or variant thereof.

In some embodiments, the AAV particle described herein is useful for the treatment, prophylaxis, palliation, or amelioration of a Niemann-Pick disorder, e.g., a Niemann-Pick A or a Niemann-Pick C1 (NPC-1). In some embodiments, treatment of a Niemann-Pick disorder, e.g., a Niemann-Pick A or a Niemann-Pick C1 (NPC-1) comprises the use of an AAV particle described herein for a gene replacement therapy. In some embodiments, the payload encoded by an AAV particle comprising a capsid polypeptide described herein comprises an ASM protein, an NPC1 protein, or variant thereof.

In some embodiments, the AAV particle described herein is useful for the treatment, prophylaxis, palliation, or amelioration of Schwannoma (e.g., Neuroma). In some embodiments, treatment of Schwannoma (e.g., Neuroma) comprises the use of an AAV particle described herein for a gene replacement therapy. In some embodiments, the payload encoded by an AAV particle comprising a capsid polypeptide described herein comprises a Caspase-1 protein or variant thereof.

In some embodiments, the AAV particle described herein is useful for the treatment, prophylaxis, palliation, or amelioration of a Tuberous Sclerosis, e.g., Tuberous Sclerosis Type 1 or Tuberous Sclerosis Type 2. In some embodiments, treatment of Tuberous Sclerosis, e.g., Tuberous Sclerosis Type 1 or Tuberous Sclerosis Type 2 comprises the use of an AAV particle described herein for a gene replacement therapy. In some embodiments, the payload encoded by an AAV particle comprising a capsid polypeptide described herein comprises a TSC1 protein, a TSC2 protein, or variant thereof.

In some embodiments, the AAV particle described herein is useful for the treatment, prophylaxis, palliation, or amelioration of a CDKL5 Deficiency. In some embodiments, treatment of a CDKL5 Deficiency comprises the use of an AAV particle described herein for a gene replacement therapy. In some embodiments, the payload encoded by an AAV particle comprising a capsid polypeptide described herein comprises a CDKL5 protein or variant thereof.

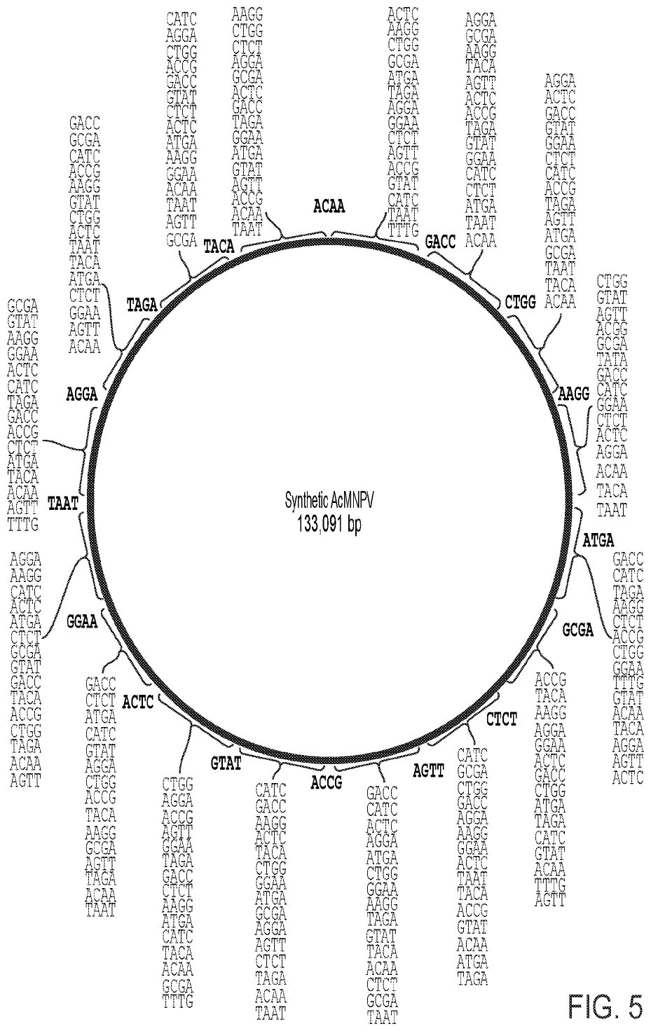

In some embodiments, the AAV particle described herein is useful for the treatment, prophylaxis, palliation, or amelioration of a Charcot-Marie-Tooth disorder, e.g., a Charcot-Marie-Tooth Type 1X (CMT1X) disorder, a Charcot-Marie-Tooth Type 2A (CMT2A) disorder, or a Charcot-Marie-Tooth Type 4J (CMT4J) disorder. In some embodiments, treatment of a Charcot-Marie-Tooth disorder, e.g., a Charcot-Marie-Tooth Type 1X (CMT1X) disorder, a Charcot-Marie-Tooth Type 2A (CMT2A) disorder, or a Charcot-Marie-Tooth Type 4J (CMT4J) disorder, comprises the use of an AAV particle described herein for a gene replacement therapy. In some embodiments, the payload encoded by an AAV particle comprising a capsid polypeptide described herein comprises a GJB1 protein, a MFN2 protein, a FIG. 4 protein, or variant thereof.

In some embodiments, the AAV particle described herein is useful for the treatment, prophylaxis, palliation, or amelioration of an Aspartylglucosaminuria (AGU). In some embodiments, treatment of an AGU comprises the use of an AAV particle described herein for a gene replacement therapy. In some embodiments, the payload encoded by an AAV particle comprising a capsid polypeptide described herein comprises an AGA protein or variant thereof.

In some embodiments, the AAV particle described herein is useful for the treatment, prophylaxis, palliation, or amelioration of a Leigh Syndrome. In some embodiments, treatment of a Leigh Syndrome comprises the use of an AAV particle described herein for a gene replacement therapy. In some embodiments, the payload encoded by an AAV particle comprising a capsid polypeptide described herein comprises a SURF1 protein or variant thereof.

In some embodiments, the AAV particle described herein is useful for the treatment, prophylaxis, palliation, or amelioration of epilepsy. In some embodiments, treatment of epilepsy comprises the use of an AAV particle described herein for a gene replacement therapy. In some embodiments, the payload encoded by an AAV particle comprising a capsid polypeptide described herein comprises an NPY/Y2 protein, a Galanin protein, a Dynorphin protein, an AIMP2-DX2 protein, an SLC6A1 protein, an SLC13A5 protein, a KCNQ2 protein, or variant thereof.

In some embodiments, the AAV particle described herein is useful for the treatment, prophylaxis, palliation, or amelioration of a Dravet Syndrome. In some embodiments, treatment of Dravet Syndrome comprises the use of an AAV particle described herein for a gene replacement therapy. In some embodiments, the payload encoded by an AAV particle comprising a capsid polypeptide described herein comprises an SCN1a protein, or variant thereof.

In some embodiments, the AAV particle described herein is useful for the treatment, prophylaxis, palliation, or amelioration of a Duchenne muscular dystrophy (DMD). In some embodiments, treatment of DMD comprises the use of an AAV particle described herein for a gene replacement therapy or enhancement (e.g., correction of exon-skipping), or a gene editing therapy (e.g., enhancement or correction). In some embodiments, the payload encoded or corrected by an AAV particle comprising a capsid polypeptide described herein comprises a Dystrophin gene and/or protein, a Utrophin gene and/or protein, or a GALGT2 gene and/or protein, or a Follistatin gene and/or protein, or a combination or variant thereof.

In some embodiments, the AAV particle described herein is useful for the treatment, prophylaxis, palliation, or amelioration of Pompe Disease. In some embodiments, treatment of Pompe Disease comprises the use of an AAV particle described herein for a gene replacement therapy. In some embodiments, the payload encoded by an AAV particle comprising a capsid polypeptide described herein comprises a GAA protein, or variant thereof.

In some embodiments, the AAV particle described herein is useful for the treatment, prophylaxis, palliation, or amelioration of Limb-Girdle Muscular Dystrophy (LGMD2A). In some embodiments, treatment of LGMD2A comprises the use of an AAV particle described herein for a gene replacement therapy. In some embodiments, the payload encoded by an AAV particle comprising a capsid polypeptide described herein comprises a CAPN-3 protein, DYSF protein, a SGCG protein, a SGCA protein, a SGCB protein, a FKRP protein, a ANO5 protein, or a combination or variant thereof.

In some embodiments, the AAV particle described herein is useful for the treatment, prophylaxis, palliation, or amelioration of chronic or neuropathic pain.

In some embodiments, the AAV particle described herein is useful for treatment, prophylaxis, palliation, or amelioration of a disease associated with the central nervous system.

In some embodiments, the AAV particle described herein is useful for treatment, prophylaxis, palliation, or amelioration of a disease associated with the peripheral nervous system.

In some embodiments, the AAV particle described herein is useful for treatment, prophylaxis, palliation, or amelioration of a neuro-oncological disorder in a subject. In some embodiments, treatment of a neuro-oncological disorder comprises prevention of said neuro-oncological disorder. In some embodiments, a neuro-oncological disorder comprises a cancer of a primary CNS origin (e.g., a CNS cell, a tissue, or a region), or a metastatic cancer in a CNS cell, tissue, or region. Examples of primary CNS cancers could be gliomas (which may include glioblastoma (also known as glioblastoma multiforme), astrocytomas, oligodendrogliomas, and ependymomas, and mixed gliomas), meningiomas, medulloblastomas, neuromas, and primary CNS lymphoma (in the brain, spinal cord, or meninges), among others. Examples of metastatic cancers include those originating in another tissue or organ, e.g., breast, lung, lymphoma, leukemia, melanoma (skin cancer), colon, kidney, prostate, or other types that metastasize to brain.

In some embodiments, the AAV particle described herein is useful for the treatment, prophylaxis, palliation, or amelioration of a disease associated with expression of HER2, e.g., a disease associated with overexpression of HER2. In some embodiments, the AAV particle described herein is useful for the treatment, prophylaxis, palliation, or amelioration of a HER2-positive cancer. In some embodiments, the HER2-positive cancer is a HER2-positive solid tumor. Additionally, or alternatively, the HER2-positive cancer may be a locally advanced or metastatic HER2-positive cancer. In some instances, the HER2-positive cancer is a HER2-positive breast cancer or a HER2-positive gastric cancer. In some embodiments, the HER2-positive cancer is selected from the group consisting of a HER2-positive gastroesophageal junction cancer, a HER2-positive colorectal cancer, a HER2-positive lung cancer (e.g., a HER2-positive non-small cell lung carcinoma), a HER2-positive pancreatic cancer, a HER2-positive colorectal cancer, a HER2-positive bladder cancer, a HER2-positive salivary duct cancer, a HER2-positive ovarian cancer (e.g., a HER2-positive epithelial ovarian cancer), or a HER2-positive endometrial cancer. In some instances, the HER2-positive cancer is prostate cancer. In some embodiments, the HER2-positive cancer has metastasized to the central nervous system (CNS). In some instances, the metastasized HER2-cancer has formed CNS neoplasms.

In some embodiments, the AAV particle described herein is useful for treatment, prophylaxis, palliation, or amelioration of a muscular disorder and/or neuromuscular disorder in a subject. In some embodiments, treatment of a muscular disorder and/or neuromuscular disorder comprises prevention of said muscular disorder and/or neuromuscular disorder.

In some embodiments, the AAV particle described herein is useful for treatment, prophylaxis, palliation or amelioration of a cardiac disease or heart disease and/or method of improving (e.g., enhancing) cardiac function in a subject. In some embodiments, the cardiac disease is a cardiomyopathy (e.g., arrhythmogenic right ventricular cardiomyopathy, dilated cardiomyopathy, or hypertrophic cardiomyopathy), congestive heart failure, tachycardia (e.g., catecholaminergic polymorphic ventricular tachycardia), ischemic heart disease, and/or myocardial infarction. In some embodiments, the cardiac disease is a disease associated with expression, e.g., aberrant expression, of LAMP2B, MYBPC3, TNNI3, LMNA, BAG3, DWORF, PKP2, Cx43, TAZ, CASQ2, SERCA2a, I-1c, S100A1 and/or ARC, S100A1, ASCL1, miR133, Mydelta3, Sav, or a combination or variant thereof. In some embodiments, treatment of a cardiac disorder described herein comprises the use of an AAV particle described herein for a gene replacement therapy.

In some embodiments, the cardiac disease is a genetic disorder, e.g., an autosomal dominant genetic disorder, an autosomal recessive disorder, or an X-linked recessive genetic disorder. In some embodiments, the cardiomyopathy is a genetic disorder, e.g., a genetic disorder associated with an abnormality (e.g., mutation, insertion, rearrangement and/or deletion) in a gene chosen from TTN, LMNA, MYH7, MYH6, SCN5A, TNNT2, RBM20, TNNI3, MYL2, MYL3, PKP2, DSP, DSG2, DSC2, JUP, or a combination thereof. In some embodiments, the cardiac disorder is a dilated cardiomyopathy, e.g., a dilated cardiomyopathy associated with an abnormality (e.g., mutation, insertion, rearrangement and/or deletion) in a gene chosen from TTN, LMNA, MIH7, BAG3, MIPN, TNNT2, SCN5A, RBN20, TNPO, LAMA4, VCL, LDB3, TCAP, PSEN1/2, ACTN2, CRYAB, TPM1, ABCC9, ACTC1, PDLIM3, ILK, TNNC1, TNNI3, PLN, DES, SGCD, CSRP3, MIH6, EYA4, ANKRD1, DMD, GATADI, TAZ/G4.5, or combination thereof. In some embodiments, the cardiac disorder is a hypertrophic cardiomyopathy, e.g., a hypertrophic cardiomyopathy associated with an abnormality (e.g., mutation, insertion, rearrangement and/or deletion) in a gene chosen from MYH7, TNNT2, TNNI3, TPM1, MYL2, MYL3, ACTC1, CSRP3, TTN, ACTN2, MYH6, TCAP, TNNC1, or a combination thereof. In some embodiments, the cardiac disorder is an arrhythmogenic ventricular cardiomyopathy, e.g., an arrhythmogenic ventricular cardiomyopathy associated with an abnormality (e.g., mutation, insertion, rearrangement and/or deletion) in a gene chosen from PKP2, DSG2, DSP, RYR2, DSC2, TGFB3, TMEM43, DES, TTN, LMNA, or a combination thereof.

In some embodiments, the AAV particle described herein is administered to a subject having at least one of the diseases or symptoms described herein. In some embodiments, an AAV particle of the present disclosure is administered to a subject having or diagnosed with having a disease or disorder described herein.

Any neurological disease or disorder, neurodegenerative disorder, muscular disorder, neuromuscular disorder, and/or neuro-oncological disorder may be treated with the AAV particles of the disclosure, or pharmaceutical compositions thereof, including but not limited to, Absence of the Septum Pellucidum, Acid Lipase Disease, Acid Maltase Deficiency, Acquired Epileptiform Aphasia, Acute Disseminated Encephalomyelitis, Attention Deficit-Hyperactivity Disorder (ADHD), Adie's Pupil, Adie's Syndrome, Adrenoleukodystrophy, Agenesis of the Corpus Callosum, Agnosia, Aicardi Syndrome, Aicardi-Goutieres Syndrome Disorder, AIDS—Neurological Complications, Alexander Disease, Alpers' Disease, Alternating Hemiplegia, Alzheimer's Disease, Amyotrophic Lateral Sclerosis (ALS), Anencephaly, Aneurysm, Angelman Syndrome, Angiomatosis, Anoxia, Antiphospholipid Syndrome, Aphasia, Apraxia, Arachnoid Cysts, Arachnoiditis, Arnold-Chiari Malformation, Arteriovenous Malformation, Asperger Syndrome, Ataxia, Ataxia Telangiectasia, Ataxias and Cerebellar or Spinocerebellar Degeneration, Atrial Fibrillation and Stroke, Attention Deficit-Hyperactivity Disorder, Autism Spectrum Disorder, Autonomic Dysfunction, Back Pain, Barth Syndrome, Batten Disease, Becker's Myotonia, Bechet's Disease, Bell's Palsy, Benign Essential Blepharospasm, Benign Focal Amyotrophy, Benign Intracranial Hypertension, Bernhardt-Roth Syndrome, Binswanger's Disease, Blepharospasm, Bloch-Sulzberger Syndrome, Brachial Plexus Birth Injuries, Brachial Plexus Injuries, Bradbury-Eggleston Syndrome, Brain and Spinal Tumors, Brain Aneurysm, Brain Injury, Brown-Sequard Syndrome, Bulbar palsy, Bulbospinal Muscular Atrophy, Cerebral Autosomal Dominant Arteriopathy with Sub-cortical Infarcts and Leukoencephalopathy (CADASIL), Canavan Disease, Carpal Tunnel Syndrome, Causalgia, Cavernomas, Cavernous Angioma, Cavernous Malformation, Central Cervical Cord Syndrome, Central Cord Syndrome, Central Pain Syndrome, Central Pontine Myelinolysis, Cephalic Disorders, Ceramidase Deficiency, Cerebellar Degeneration, Cerebellar Hypoplasia, Cerebral Aneurysms, Cerebral Arteriosclerosis, Cerebral Atrophy, Cerebral Beriberi, Cerebral Cavernous Malformation, Cerebral Gigantism, Cerebral Hypoxia, Cerebral Palsy, Cerebro-Oculo-Facio-Skeletal Syndrome (COFS), Charcot-Marie-Tooth Disease, Chiari Malformation, Cholesterol Ester Storage Disease, Chorea, Choreoacanthocytosis, Chronic Inflammatory Demyelinating Polyneuropathy (CIDP), Chronic Orthostatic Intolerance, Chronic Pain, Cockayne Syndrome Type II, Coffin Lowry Syndrome, Colpocephaly, Coma, Complex Regional Pain Syndrome, Concentric sclerosis (Baló's sclerosis), Congenital Facial Diplegia, Congenital Myasthenia, Congenital Myopathy, Congenital Vascular Cavernous Malformations, Corticobasal Degeneration, Cranial Arteritis, Craniosynostosis, Cree encephalitis, Creutzfeldt-Jakob Disease, Chronic progressive external ophtalmoplegia, Cumulative Trauma Disorders, Cushing's Syndrome, Cytomegalic Inclusion Body Disease, Cytomegalovirus Infection, Dancing Eyes-Dancing Feet Syndrome, Dandy-Walker Syndrome, Dawson Disease, De Morsier's Syndrome, Dejerine-Klumpke Palsy, Dementia, Dementia-Multi-Infarct, Dementia—Semantic, Dementia-Subcortical, Dementia With Lewy Bodies, Demyelination diseases, Dentate Cerebellar Ataxia, Dentatorubral Atrophy, Dermatomyositis, Developmental Dyspraxia, Devic's Syndrome, Diabetic Neuropathy, Diffuse Sclerosis, Distal hereditary motor neuronopathies, Dravet Syndrome, Dysautonomia, Dysgraphia, Dyslexia, Dysphagia, Dyspraxia, Dyssynergia Cerebellaris Myoclonica, Dyssynergia Cerebellaris Progressiva, Dystonias, Early Infantile Epileptic Encephalopathy, Empty Sella Syndrome, Encephalitis, Encephalitis Lethargica, Encephaloceles, Encephalomyelitis, Encephalopathy, Encephalopathy (familial infantile), Encephalotrigeminal Angiomatosis, Epilepsy, Epileptic Hemiplegia, Episodic ataxia, Erb's Palsy, Erb-Duchenne and Dejerine-Klumpke Palsies, Essential Tremor, Extrapontine Myelinolysis, Faber's disease, Fabry Disease, Fahr's Syndrome, Fainting, Familial Dysautonomia, Familial Hemangioma, Familial Idiopathic Basal Ganglia Calcification, Familial Periodic Paralyses, Familial Spastic Paralysis, Farber's Disease, Febrile Seizures, Fibromuscular Dysplasia, Fisher Syndrome, Floppy Infant Syndrome, Foot Drop, Friedreich's Ataxia, Frontotemporal Dementia, Gaucher Disease, Generalized Gangliosidoses (GM1, GM2), Gerstmann's Syndrome, Gerstmann-Straussler-Scheinker Disease, Giant Axonal Neuropathy, Giant Cell Arteritis, Giant Cell Inclusion Disease, Globoid Cell Leukodystrophy, Glossopharyngeal Neuralgia, Glycogen Storage Disease, Guillain-Barré Syndrome, Hallervorden-Spatz Disease, Head Injury, Headache, Hemicrania Continua, Hemifacial Spasm, Hemiplegia Alterans, Hereditary Neuropathies, Hereditary Spastic Paraplegia, Heredopathia Atactica Polyneuritiformis, Herpes Zoster, Herpes Zoster Oticus, Hirayama Syndrome, Holmes-Adie syndrome, Holoprosencephaly, HTLV-1 Associated Myelopathy, Hughes Syndrome, Huntington's Disease, Hurler syndrome, Hydranencephaly, Hydrocephalus, Hydrocephalus—Normal Pressure, Hydromyelia, Hypercortisolism, Hypersomnia, Hypertonia, Hypotonia, Hypoxia, Immune-Mediated Encephalomyelitis, Inclusion Body Myositis, Incontinentia Pigmenti, Infantile Hypotonia, Infantile Neuroaxonal Dystrophy, Infantile Phytanic Acid Storage Disease, Infantile Refsum Disease, Infantile Spasms, Inflammatory Myopathies, Iniencephaly, Intestinal Lipodystrophy, Intracranial Cysts, Intracranial Hypertension, Isaacs' Syndrome, Joubert Syndrome, Kearns-Sayre Syndrome, Kennedy's Disease, Kinsbourne syndrome, Kleine-Levin Syndrome, Klippel-Feil Syndrome, Klippel-Trenaunay Syndrome (KTS), Kluver-Bucy Syndrome, Korsakoffs Amnesic Syndrome, Krabbe Disease, Kugelberg-Welander Disease, Kuru, Lambert-Eaton Myasthenic Syndrome, Landau-Kleffner Syndrome, Lateral Femoral Cutaneous Nerve Entrapment, Lateral Medullary Syndrome, Learning Disabilities, Leigh's Disease, Lennox-Gastaut Syndrome, Lesch-Nyhan Syndrome, Leukodystrophy, Levine-Critchley Syndrome, Lewy Body Dementia, Lichtheim's disease, Lipid Storage Diseases, Lipoid Proteinosis, Lissencephaly, Locked-In Syndrome, Lou Gehrig's Disease, Lupus—Neurological Sequelae, Lyme Disease—Neurological Complications, Lysosomal storage disorders, Machado-Joseph Disease, Macrencephaly, Megalencephaly, Melkersson-Rosenthal Syndrome, Meningitis, Meningitis and Encephalitis, Menkes Disease, Meralgia Paresthetica, Metachromatic Leukodystrophy, Microcephaly, Migraine, Miller Fisher Syndrome, Mini Stroke, Mitochondrial Myopathy, Mitochondrial DNA depletion syndromes, Moebius Syndrome, Monomelic Amyotrophy, Morvan Syndrome, Motor Neuron Diseases, Moyamoya Disease, Mucolipidoses, Mucopolysaccharidoses, Multi-Infarct Dementia, Multifocal Motor Neuropathy, Multiple Sclerosis, Multiple System Atrophy, Multiple System Atrophy with Orthostatic Hypotension, Muscular Dystrophy, Myasthenia—Congenital, Myasthenia Gravis, Myelinoclastic Diffuse Sclerosis, Myelitis, Myoclonic Encephalopathy of Infants, Myoclonus, Myoclonus epilepsy, Myopathy, Myopathy-Congenital, Myopathy-Thyrotoxic, Myotonia, Myotonia Congenita, Narcolepsy, NARP (neuropathy, ataxia and retinitis pigmentosa), Neuroacanthocytosis, Neurodegeneration with Brain Iron Accumulation, Neurodegenerative disease, Neurofibromatosis, Neuroleptic Malignant Syndrome, Neurological Complications of AIDS, Neurological Complications of Lyme Disease, Neurological Consequences of Cytomegalovirus Infection, Neurological Manifestations of Pompe Disease, Neurological Sequelae Of Lupus, Neuromyelitis Optica, Neuromyotonia, Neuronal Ceroid Lipofuscinosis, Neuronal Migration Disorders, Neuropathic pain, Neuropathy-Hereditary, Neuropathy, Neurosarcoidosis, Neurosyphilis, Neurotoxicity, Nevus Cavernosus, Niemann-Pick Disease, O'Sullivan-McLeod Syndrome, Occipital Neuralgia, Ohtahara Syndrome, Olivopontocerebellar Atrophy, Opsoclonus Myoclonus, Orthostatic Hypotension, Overuse Syndrome, Pain-Chronic, Pantothenate Kinase-Associated Neurodegeneration, Paraneoplastic Syndromes, Paresthesia, Parkinson's Disease, Paroxysmal Choreoathetosis, Paroxysmal Hemicrania, Parry-Romberg, Pelizaeus-Merzbacher Disease, Pena Shokeir II Syndrome, Perineural Cysts, Peroneal muscular atrophy, Periodic Paralyses, Peripheral Neuropathy, Periventricular Leukomalacia, Persistent Vegetative State, Pervasive Developmental Disorders, Phytanic Acid Storage Disease, Pick's Disease, Pinched Nerve, Piriformis Syndrome, Pituitary Tumors, Polymyositis, Pompe Disease, Porencephaly, Post-Polio Syndrome, Postherpetic Neuralgia, Postinfectious Encephalomyelitis, Postural Hypotension, Postural Orthostatic Tachycardia Syndrome, Postural Tachycardia Syndrome, Primary Dentatum Atrophy, Primary Lateral Sclerosis, Primary Progressive Aphasia, Prion Diseases, Progressive bulbar palsy, Progressive Hemifacial Atrophy, Progressive Locomotor Ataxia, Progressive Multifocal Leukoencephalopathy, Progressive Muscular Atrophy, Progressive Sclerosing Poliodystrophy, Progressive Supranuclear Palsy, Prosopagnosia, Pseudobulbar palsy, Pseudo-Torch syndrome, Pseudotoxoplasmosis syndrome, Pseudotumor Cerebri, Psychogenic Movement, Ramsay Hunt Syndrome I, Ramsay Hunt Syndrome II, Rasmussen's Encephalitis, Reflex Sympathetic Dystrophy Syndrome, Refsum Disease, Refsum Disease—Infantile, Repetitive Motion Disorders, Repetitive Stress Injuries, Restless Legs Syndrome, Retrovirus-Associated Myelopathy, Rett Syndrome, Reye's Syndrome, Rheumatic Encephalitis, Riley-Day Syndrome, Sacral Nerve Root Cysts, Saint Vitus Dance, Salivary Gland Disease, Sandhoff Disease, Schilder's Disease, Schizencephaly, Seitelberger Disease, Seizure Disorder, Semantic Dementia, Septo-Optic Dysplasia, Severe Myoclonic Epilepsy of Infancy (SMEI), Shaken Baby Syndrome, Shingles, Shy-Drager Syndrome, Sjögren's Syndrome, Sleep Apnea, Sleeping Sickness, Sotos Syndrome, Spasticity, Spina Bifida, Spinal Cord Infarction, Spinal Cord Injury, Spinal Cord Tumors, Spinal Muscular Atrophy, Spinocerebellar Ataxia, Spinocerebellar Atrophy, Spinocerebellar Degeneration, Sporadic ataxia, Steele-Richardson-Olszewski Syndrome, Stiff-Person Syndrome, Striatonigral Degeneration, Stroke, Sturge-Weber Syndrome, Subacute Sclerosing Panencephalitis, Subcortical Arteriosclerotic Encephalopathy, Short-lasting, Unilateral, Neuralgiform (SUNCT) Headache, Swallowing Disorders, Sydenham Chorea, Syncope, Syphilitic Spinal Sclerosis, Syringohydromyelia, Syringomyelia, Systemic Lupus Erythematosus, Tabes Dorsalis, Tardive Dyskinesia, Tarlov Cysts, Tay-Sachs Disease, Temporal Arteritis, Tethered Spinal Cord Syndrome, Thomsen's Myotonia, Thoracic Outlet Syndrome, Thyrotoxic Myopathy, Tic Douloureux, Todd's Paralysis, Tourette Syndrome, Transient Ischemic Attack, Transmissible Spongiform Encephalopathies, Transverse Myelitis, Traumatic Brain Injury, Tremor, Trigeminal Neuralgia, Tropical Spastic Paraparesis, Troyer Syndrome, Tuberous Sclerosis, Vascular Erectile Tumor, Vasculitis Syndromes of the Central and Peripheral Nervous Systems, Vitamin B12 deficiency, Von Economo's Disease, Von Hippel-Lindau Disease (VHL), Von Recklinghausen's Disease, Wallenberg's Syndrome, Werdnig-Hoffman Disease, Wernicke-Korsakoff Syndrome, West Syndrome, Whiplash, Whipple's Disease, Williams Syndrome, Wilson Disease, Wolman's Disease, X-Linked Spinal and Bulbar Muscular Atrophy.

### Pharmaceutical Compositions and Formulations

Provided herein are pharmaceutical compositions comprising an AAV particle described herein. In some embodiments, the pharmaceutical composition comprises at least one active ingredients. In some embodiments, the pharmaceutical composition comprises a pharmaceutically acceptable excipient.

In some embodiments, an AAV particle described herein is formulated using an excipient to: (1) increase stability; (2) increase cell transfection or transduction; (3) permit the sustained or delayed expression of the payload; (4) alter the biodistribution (e.g., target the viral particle to specific tissues or cell types); (5) increase the translation of encoded protein; (6) alter the release profile of encoded protein; and/or (7) allow for regulatable expression of the payload. Formulations of the present disclosure can include, without limitation, saline, liposomes, lipid nanoparticles, polymers, peptides, proteins, cells transfected with viral vectors (e.g., for transfer or transplantation into a subject) and combinations thereof.

In some embodiments, the relative amount of the active ingredient (e.g. an AAV particle described herein), a pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1% and 99% (w/w) of the active ingredient. By way of example, the composition may comprise between 0.1% and 100%, e.g., between 0.5 and 50%, between 1-30%, between 5-80%, at least 80% (w/w) active ingredient.

In some embodiments, the pharmaceutical composition comprising an AAV particle described herein may comprise an AAV capsid polypeptide and a viral genome encoding a payload, e.g., a payload described herein, with or without a pharmaceutically acceptable excipient.

The present disclosure also provides in some embodiments, a pharmaceutical composition suitable for administration to a subject, e.g., a human. In some embodiments, the pharmaceutical composition is administered to a subject, e.g., a human.

### Kits

Also provided herein are kits comprising an AAV particle described herein, e.g., an AAV particle produced using a baculovirus (or baculoviruses) produced using a variant baculovirus genome, a baculovirus expression construct, a bacmid, and/or a BAC described herein, and instructions for use.

### Administration

In some embodiments, an AAV particle disclosed herein may be administered by a to a subject by a delivery route, e.g., a localized delivery route or a systemic delivery route.

In some embodiments, an AAV particle described herein may be administered via such a route that it is able to cross the blood-brain barrier, vascular barrier, or other epithelial barrier. In some embodiments, an AAV particle described herein may be administered in any suitable form, either as a liquid solution or suspension, as a solid form suitable for liquid solution or suspension in a liquid solution. In some embodiments, an AAV particle may be formulated with any appropriate and pharmaceutically acceptable excipient.

In some embodiments, the AAV particle described herein is administered intramuscularly, intravenously, intracerebrally, intrathecally, intracerebroventricularly, via intraparenchymal administration, or via intra-cisterna magna injection (ICM).

In some embodiments, an AAV particle described herein may be delivered to a subject via a single route administration. In some embodiments, an AAV particle described herein may be delivered to a subject via a multi-site route of administration. In some embodiments, a subject may be administered at 2, 3, 4, 5, or more than 5 sites.

In some embodiments, an AAV particle described herein is administered via a bolus infusion. In some embodiments, an AAV particle of the present disclosure is administered via sustained delivery over a period of minutes, hours, or days. In some embodiments, the infusion rate may be changed depending on the subject, distribution, formulation, and/or another delivery parameter. In some embodiments, an AAV particle described herein is administered using a controlled release. In some embodiments, an AAV particle described herein is administered using a sustained release, e.g., a release profile that conforms to a release rate over a specific period of time.

In some embodiments, an AAV particle described herein may be delivered by more than one route of administration. As non-limiting examples of combination administrations, an AAV particle may be delivered by intrathecal and intracerebroventricular, or by intravenous and intraparenchymal administration.

Intravenous administration

In some embodiments, an AAV particle described herein may be administered to a subject by systemic administration. In some embodiments, the systemic administration is intravenous administration. In another embodiment, the systemic administration is intraarterial administration. In some embodiments, an AAV particle described herein may be administered to a subject by intravenous administration. In some embodiments, the intravenous administration may be achieved by subcutaneous delivery. In some embodiments, the AAV particle is administered to the subject via focused ultrasound (FUS), e.g., coupled with the intravenous administration of microbubbles (FUS-MB) or MRI-guided FUS coupled with intravenous administration, e.g., as described in Terstappen et al. (Nat Rev Drug Discovery, https://doi.org/10.1038/s41573-021-00139-y (2021)), the contents of which are incorporated herein by reference in its entirety. In some embodiments, the AAV particle is administered to the subject intravenously. In some embodiments, the subject is a human.

### Administration to the CNS

In some embodiments, an AAV particle described herein may be delivered by direct injection into the brain. As a non-limiting example, the brain delivery may be by intrahippocampal administration. In some embodiments, an AAV particle described herein may be administered to a subject by intraparenchymal administration. In some embodiments, the intraparenchymal administration is to tissue of the central nervous system. In some embodiments, an AAV particle described herein may be administered to a subject by intracranial delivery (See, e.g., U.S. Pat. No. 8,119,611; the content of which is incorporated herein by reference in its entirety). In some embodiments, an AAV particle described herein may be delivered by injection into the CSF pathway. Non-limiting examples of delivery to the CSF pathway include intrathecal and intracerebroventricular administration. In some embodiments, an AAV particle described herein may be administered via intracisternal magna (ICM) injection.

In some embodiments, an AAV particle described herein may be delivered to the brain by systemic delivery. As a non-limiting example, the systemic delivery may be by intravascular administration. As a non-limiting example, the systemic or intravascular administration may be intravenous.

In some embodiments, an AAV particle described herein may be delivered by an intraocular delivery route. A non-limiting example of an intraocular administration includes an intravitreal injection.

### Intramuscular Administration

In some embodiments, an AAV particle described herein may be delivered by intramuscular administration. Without wishing to be bound by theory, it is believed in some embodiments, that the multi-nucleated nature of muscle cells provides an advantage to gene transduction subsequent to AAV delivery. In some embodiments, cells of the muscle are capable of expressing recombinant proteins with the appropriate post-translational modifications. Without wishing to be bound by theory, it is believed in some embodiments, the enrichment of muscle tissue with vascular structures allows for transfer to the blood stream and whole-body delivery. Examples of intramuscular administration include systemic (e.g., intravenous), subcutaneous or directly into the muscle. In some embodiments, more than one injection is administered. In some embodiments, an AAV particle described herein may be delivered by an intramuscular delivery route. (See, e.g., U.S. Pat. No. 6,506,379; the content of which is incorporated herein by reference in its entirety). Non-limiting examples of intramuscular administration include an intravenous injection or a subcutaneous injection.

In some embodiments, an AAV particle described herein is administered to a subject and transduces the muscle of a subject. As a non-limiting example, an AAV particle is administered by intramuscular administration. In some embodiments, an AAV particle described herein may be administered to a subject by subcutaneous administration. In some embodiments, the intramuscular administration is via systemic delivery. In some embodiments, the intramuscular administration is via intravenous delivery. In some embodiments, the intramuscular administration is via direct injection to the muscle.

In some embodiments, the muscle is transduced by administration, e.g., intramuscular administration. In some embodiments, an intramuscular delivery comprises administration at one site. In some embodiments, an intramuscular delivery comprises administration at more than one site. In some embodiments, an intramuscular delivery comprises administration at two, three, four, or more sites. In some embodiments, intramuscular delivery is combined with at least one other method of administration.

In some embodiments, an AAV particle described herein may be administered to a subject by peripheral injections. Non-limiting examples of peripheral injections include intraperitoneal, intramuscular, intravenous, conjunctival, or joint injection. It was disclosed in the art that the peripheral administration of AAV vectors can be transported to the central nervous system, for example, to the motor neurons (e.g., U.S. Patent Publication Nos. US20100240739 and US20100130594; the content of each of which is incorporated herein by reference in their entirety).

In some embodiments, an AAV particle described herein may be administered to a subject by intraparenchymal administration. In some embodiments, the intraparenchymal administration is to muscle tissue. In some embodiments, an AAV particle described herein is delivered as described in Bright et al 2015 (Neurobiol Aging. 36(2):693-709), the contents of which are herein incorporated by reference in their entirety. In some embodiments, an AAV particle described herein is administered to the gastrocnemius muscle of a subject. In some embodiments, an AAV particle described herein is administered to the bicep femorii of the subject. In some embodiments, an AAV particles described herein is administered to the tibialis anterior muscles. In some embodiments, an AAV particle described herein is administered to the soleus muscle.

### Depot Administration

In some embodiments, a pharmaceutical composition and/or an AAV particle described herein (e.g., an AAV particle comprising an AAV capsid polypeptide) are formulated in depots for extended release. Generally, specific organs or tissues are targeted for administration.

In some embodiments, a pharmaceutical composition and/or an AAV particle described herein (e.g., an AAV particle comprising an AAV capsid polypeptide) are spatially retained within or proximal to target tissues. Provided are methods of providing a pharmaceutical composition, an AAV particle, to target tissues of mammalian subjects by contacting target tissues (which comprise one or more target cells) with the pharmaceutical composition and/or the AAV particle, under conditions such that they are substantially retained in target tissues, e.g., such that at least 10, 20, 30, 40, 50, 60, 70, 80, 85, 90, 95, 96, 97, 98, 99, 99.9, 99.99 or greater than 99.99% of the composition is retained in the target tissues. In some embodiments, retention is determined by measuring the amount of pharmaceutical composition and/or AAV particle, that enter a target cell or a plurality of target cells. For example, at least 1%, 5%, 10%, 20%, 30%, 40%, 50%, 60%, 70%, 80%, 85%, 90%, 95%, 96%, 97%, 98%, 99%, 99.9%, 99.99%, or greater than 99.99% of a pharmaceutical composition and/or an AAV particle, administered to a subject are present intracellularly at a period of time following administration. For example, intramuscular injection to a subject may be performed using aqueous compositions comprising a pharmaceutical composition and/or an AAV particle described herein and a transfection reagent, and retention is determined by measuring the amount of the pharmaceutical composition and/or the AAV particle, present in the muscle cell or plurality of muscle cells.

In some embodiments, provided are methods of providing a pharmaceutical composition and/or an AAV particle described herein (e.g., an AAV particle comprising an AAV capsid polypeptide) to a tissue of a subject, by contacting the tissue (comprising a cell, e.g., a plurality of cells) with the pharmaceutical composition and/or the AAV particle under conditions such that they are substantially retained in the tissue. In some embodiments, a pharmaceutical composition and/or AAV particle described herein comprise a sufficient amount of an active ingredient such that the effect of interest is produced in at least one cell. In some embodiments, a pharmaceutical composition and/or an AAV particle generally comprise one or more cell penetration agents. In some embodiments, the disclosure provides a naked formulations (such as without cell penetration agents or other agents), with or without pharmaceutically acceptable carriers.

### Methods of Treatment

Provided in the present disclosure are methods for introducing (e.g., delivering) an AAV particle described herein (e.g., an AAV particle comprising an AAV capsid polypeptide) into cells. In some embodiments, the method comprises introducing into said cells an AAV particle or vector described herein in an amount sufficient to modulate, e.g., increase, the production of a target gene, mRNA, and/or protein. In some embodiments, the method comprises introducing into said cells an AAV particle or vector described herein in an amount sufficient to modulate, e.g., decrease, expression of a target gene, mRNA, and/or protein. In some aspects, the cells may be neurons such as but not limited to, motor, hippocampal, entorhinal, thalamic, cortical, sensory, sympathetic, or parasympathetic neurons, and glial cells such as astrocytes, microglia, and/or oligodendrocytes. In other aspects, the cells may be a muscle cell, e.g., a cell of a diaphragm, a quadriceps, or a heart (e.g., a heart atrium or a heart ventricle).

Disclosed in the present disclosure are methods for treating a neurological disease/disorder or a neurodegenerative disorder, a muscular or neuromuscular disorder, or a neurooncological disorder associated with aberrant, e.g., insufficient or increased, function/presence of a protein, e.g., a target protein in a subject in need of treatment.

In some embodiments, the method comprises administering to the subject a therapeutically effective amount of a composition comprising AAV particles described herein. As a non-limiting example, the AAV particles can increase target gene expression, increase target protein production, and thus reduce one or more symptoms of neurological disease in the subject such that the subject is therapeutically treated.

In other embodiments, the method comprises administering to the subject a therapeutically effective amount of a composition comprising AAV particles (e.g., an AAV particle comprising an AAV capsid polypeptide) comprising a viral genome with a nucleic acid sequence encoding one or more siRNA molecules. As a non-limiting example, the siRNA molecules can silence target gene expression, inhibit target protein production, and reduce one or more symptoms of neurological disease in the subject such that the subject is therapeutically treated.

In some embodiments, the composition comprising the AAV particles described is administered to the central nervous system of the subject via systemic administration. In some embodiments, the systemic administration is intravenous (IV) injection. In some embodiments, the AAV particle described herein or a pharmaceutical composition comprising an AAV particle described herein is administered by focused ultrasound (FUS), e.g., coupled with the intravenous administration of microbubbles (FUS-MB) or MRI-guided FUS coupled with intravenous administration.

In some embodiments, the composition comprising the AAV particle described herein is administered to the central nervous system of the subject via intraventricular administration. In some embodiments, the composition comprising the AAV particle described herein is administered via intra-cisterna magna injection (ICM).

In some embodiments, the composition comprising an AAV particle described herein is administered to the central nervous system of the subject via intraventricular injection and intravenous injection.

In some embodiments, the composition comprising the AAV particle described herein is administered to the central nervous system of the subject via ICM injection and intravenous injection at a specific dose per subject. As a non-limiting example, the AAV particles are administered via ICM injection at a dose of 1×10⁴ VG per subject. As a non-limiting example, the AAV particles are administered via IV injection at a dose of 2×10¹³ VG per subject.

In some embodiments, the composition comprising the AAV particle described herein is administered to the central nervous system of the subject. In other embodiments, the composition comprising the AAV particle described herein is administered to a CNS tissue of a subject (e.g., putamen, hippocampus, thalamus, or cortex of the subject).

In some embodiments, the composition comprising the AAV particle described herein is administered to the central nervous system of the subject via intraparenchymal injection. Non-limiting examples of intraparenchymal injections include intraputamenal, intracortical, intrathalamic, intrastriatal, intrahippocampal or into the entorhinal cortex.

In some embodiments, the composition comprising the AAV particle described herein is administered to the central nervous system of the subject via intraparenchymal injection and intravenous injection.

In some embodiments, the composition comprising the AAV particle described herein is administered to the central nervous system of the subject via intraventricular injection, intraparenchymal injection and intravenous injection.

In some embodiments, the composition comprising an AAV particle described herein or a plurality of particles of the present disclosure is administered to a muscle of the subject via intravenous injection. In some embodiments, the composition comprising an AAV particle of a plurality of particles of the present disclosure is administered to a muscle of the subject via intramuscular injection.

In some embodiments, an AAV particle described herein may be delivered into specific types of cells, including, but not limited to, thalamic, hippocampal, entorhinal, cortical, motor, sensory, excitatory, inhibitory, sympathetic, or parasympathetic neurons; glial cells including oligodendrocytes, astrocytes and microglia; and/or other cells surrounding neurons such as T cells. In some embodiments, an AAV particle described herein may be delivered into a muscle cell, e.g., a cell of the quadriceps, diaphragm, liver, and/or heart (e.g., heart atrium or heart ventricle).

In some embodiments, an AAV particle, e.g., a plurality of particles, described herein may be delivered to a cell or region of the midbrain. In some embodiments, an AAV particle, e.g., a plurality of particles, described herein may be delivered to a cell or region of the brains stem.

In some embodiments, an AAV particle, e.g., a plurality of particles, described herein may be delivered to neurons in the putamen, hippocampus, thalamus and/or cortex.

In some embodiments, an AAV particle, e.g., a plurality of particles, described herein may be used as a therapy for a genetic disorder, e.g., an autosomal dominant genetic disorder, an autosomal recessive disorder, X-linked dominant genetic disorder, an X-linked recessive genetic disorder, or a Y-linked genetic disorder. In some embodiments, the genetic disorder is a monogenetic disorder or a polygenic disorder. In some embodiments, treatment of a genetic disorder, e.g., a monogenic disorder, comprises the use of an AAV particle described herein for a gene replacement therapy.

In some embodiments, an AAV particle, e.g., a plurality of particles, described herein may be used as a therapy for a neurological disease.

In some embodiments, an AAV particle, e.g., a plurality of particles, described herein may be used as a therapy for tauopathies.

In some embodiments, an AAV particle, e.g., a plurality of particles, described herein may be used as a therapy for Alzheimer's Disease.

In some embodiments, an AAV particle, e.g., a plurality of particles, of the present disclosure may be used as a therapy for Amyotrophic Lateral Sclerosis.

In some embodiments, an AAV particle, e.g., a plurality of particles, described herein may be used as a therapy for Huntington's Disease.

In some embodiments, an AAV particle, e.g., a plurality of particles, described herein may be used as a therapy for Parkinson's Disease.

In some embodiments, an AAV particle, e.g., a plurality of particles, described herein may be used as a therapy for Gaucher disease (GD) (e.g., Type 1 GD, Type 2 GD, or Type 3 GD). In some embodiments, an AAV particle, e.g., a plurality of particles, described herein may be used as a therapy for Parkinson's disease associated with a GBA mutation. In some embodiments, an AAV particle, e.g., a plurality of particles, described herein may be used as a therapy for dementia with Lewy Bodies (DLB).

In some embodiments, an AAV particle, e.g., a plurality of particles, described herein may be used as a therapy for spinal muscular atrophy.

In some embodiments, an AAV particle, e.g., a plurality of particles, described herein may be used as a therapy for a leukodystrophy, e.g., Alexander disease, autosomal dominant leukodystrophy with autonomic diseases (ADLD), Canavan disease, cerebrotendinous xanthomatosis (CTX), metachromatic leukodystrophy (MLD), Pelizaeus-Merzbacher disease, or Refsum disease.

In some embodiments, an AAV particle, e.g., a plurality of particles, described herein may be used as a therapy for Friedreich's Ataxia.

In some embodiments, an AAV particle, e.g., a plurality of particles, described herein may be used as a therapy for chronic or neuropathic pain.

In some embodiments, an AAV particle, e.g., a plurality of particles, described herein may be used as a therapy for a disease associated with expression of HER2, e.g., a disease associated with overexpression of HER2. In some embodiments, the AAV particle described herein is useful for the treatment, prophylaxis, palliation or amelioration of a HER2-positive cancer. In some embodiments, the HER2-positive cancer is a HER2-positive solid tumor. Additionally, or alternatively, the HER2-positive cancer may be a locally advanced or metastatic HER2-positive cancer. In some instances, the HER2-positive cancer is a HER2-positive breast cancer or a HER2-positive gastric cancer. In some embodiments, the HER2-positive cancer is selected from the group consisting of a HER2-positive gastroesophageal junction cancer, a HER2-positive colorectal cancer, a HER2-positive lung cancer (e.g., a HER2-positive non-small cell lung carcinoma), a HER2-positive pancreatic cancer, a HER2-positive colorectal cancer, a HER2-positive bladder cancer, a HER2-positive salivary duct cancer, a HER2-positive ovarian cancer (e.g., a HER2-positive epithelial ovarian cancer), or a HER2-positive endometrial cancer. In some instances, the HER2-positive cancer is prostate cancer. In some embodiments, the HER2-positive cancer has metastasized to the central nervous system (CNS). In some instances, the metastasized HER2-cancer has formed CNS neoplasms.

In some embodiments, an AAV particle, e.g., a plurality of particles, described herein may be used as a therapy for a neuro-oncological disorder. In some embodiments, the neuro-oncological disorder is a cancer of primary CNS origin (e.g., a cancer of a CNS cell and/or CNS tissue). In some embodiments, the neuro-oncological disorder is metastatic cancer in a CNS cell, CNS region, and/or a CNS tissue. Examples of primary CNS cancers could be gliomas (which may include glioblastoma (also known as glioblastoma multiforme), astrocytomas, oligodendrogliomas, and ependymomas, and mixed gliomas), meningiomas, medulloblastomas, neuromas, and primary CNS lymphoma (in the brain, spinal cord, or meninges), among others. Examples of metastatic cancers include those originating in another tissue or organ, e.g., breast, lung, lymphoma, leukemia, melanoma (skin cancer), colon, kidney, prostate, or other types that metastasize to brain.

In some embodiments, an AAV particle, e.g., a plurality of particles, described herein may be used as a therapy for a muscular disorder or a neuromuscular disorder.

In some embodiments, an AAV particle, e.g., a plurality of particles, described herein may be used as a therapy for a cardiac disease or heart disease and/or method of improving (e.g., enhancing) cardiac function in a subject. In some embodiments, the cardiac disease is a cardiomyopathy (e.g., arrhythmogenic right ventricular cardiomyopathy, dilated cardiomyopathy, or hypertrophic cardiomyopathy), congestive heart failure, tachycardia (e.g., catecholaminergic polymorphic ventricular tachycardia), ischemic heart disease, and/or myocardial infarction.

In some embodiments, administration of the AAV particle described herein to a subject may increase target gene, mRNA, and/or protein levels in a subject, relative to a control, e.g., the gene, mRNA, and/or mRNA levels in the subject prior to receiving AAV particle. The target gene, mRNA, and/or protein levels may be increased by about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100% in a subject such as, but not limited to, the CNS, a region of the CNS, or a specific cell of the CNS, or a muscle, a region of a muscle, or a cell of a muscle, of a subject. In some embodiments, cell of the CNS comprises an astrocyte, microglia, cortical neuron, hippocampal neuron, DRG and/or sympathetic neuron, sensory neuron, oligodendrocyte, motor neuron, or combination thereof. As a non-limiting example, the AAV particles may increase the gene, mRNA, and/or protein levels of a target protein by fold increases over baseline. In some embodiments, AAV particles lead to 5-6 times higher levels of a target gene, mRNA, or protein.

In some embodiments, administration of the AAV particle described herein (e.g., an AAV particle comprising an AAV capsid polypeptide), e.g., an AAV particle comprising a nucleic acid encoding a siRNA molecule or an antibody or antibody fragment, to a subject may decrease target gene, mRNA, and/or protein levels in a subject, relative to a control, e.g., the gene, mRNA, and/or mRNA levels in the subject prior to receiving AAV particle. The target gene, mRNA, and/or protein levels may be decreased by about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100% in a subject such as, but not limited to, the CNS, a region of the CNS, or a specific cell of the CNS, or a muscle, a region of a muscle, or a cell of a muscle, of a subject. In some embodiments, cell of the CNS comprises an astrocyte, microglia, cortical neuron, hippocampal neuron, DRG and/or sympathetic neuron, sensory neuron, oligodendrocyte, motor neuron, or combination thereof. As a non-limiting example, the AAV particles may decrease the gene, mRNA, and/or protein levels of a target protein by fold decreases over baseline.

In some embodiments, the AAV particles described herein may be used to increase target protein and reduce symptoms of neurological disease in a subject. In some embodiments, the AAV particles described herein may be used to decrease target protein and reduce symptoms of neurological disease in a subject.

In some embodiments, the AAV particles described herein may be used to reduce the decline of functional capacity and activities of daily living as measured by a standard evaluation system such as, but not limited to, the total functional capacity (TFC) scale.

In some embodiments, the AAV particles described herein may be used to improve performance on any assessment used to measure symptoms of neurological disease. Such assessments include, but are not limited to ADAS-cog (Alzheimer Disease Assessment Scale—cognitive), MMSE (Mini-Mental State Examination), GDS (Geriatric Depression Scale), FAQ (Functional Activities Questionnaire), ADL (Activities of Daily Living), GPCOG (General Practitioner Assessment of Cognition), Mini-Cog, AMTS (Abbreviated Mental Test Score), Clock-drawing test, 6-CIT (6-item Cognitive Impairment Test), TYM (Test Your Memory), MoCa (Montreal Cognitive Assessment), ACE-R (Addenbrookes Cognitive Assessment), MIS (Memory Impairment Screen), BADLS (Bristol Activities of Daily Living Scale), Barthel Index, Functional Independence Measure, Instrumental Activities of Daily Living, IQCODE (Informant Questionnaire on Cognitive Decline in the Elderly), Neuropsychiatric Inventory, The Cohen-Mansfield Agitation Inventory, BEHAVE-AD, EuroQol, Short Form-36 and/or MBR Caregiver Strain Instrument, or any of the other tests as described in Sheehan B (Ther Adv Neurol Disord. 5(6):349-358 (2012)), the contents of which are herein incorporated by reference in their entirety.

In some embodiments, the present composition is administered as a solo therapeutic or as combination therapeutic for the treatment of a neurological disease/disorder or a neurodegenerative disorder, a muscular disorder or neuromuscular disorder, and/or a neuro-oncological disorder.

The AAV particles encoding the target protein may be used in combination with one or more other therapeutic agents. In some embodiments, compositions can be administered concurrently with, prior to, or subsequent to, additional therapeutic or medical procedures. In general, each agent will be administered at a dose and/or on a time schedule determined for that agent.

Therapeutic agents that may be used in combination with the AAV particles described herein can be small molecule compounds which are antioxidants, anti-inflammatory agents, anti-apoptosis agents, calcium regulators, anti-glutamatergic agents, structural protein inhibitors, compounds involved in muscle function, and compounds involved in metal ion regulation. As a non-limiting example, the combination therapy may be in combination with one or more neuroprotective agents such as small molecule compounds, growth factors and hormones which have been tested for their neuroprotective effect on motor neuron degeneration.

Compounds tested for treating neurological disease which may be used in combination with the AAV particles described herein include, but are not limited to, cholinesterase inhibitors (donepezil, rivastigmine, galantamine), NMDA receptor antagonists such as memantine, anti-psychotics, anti-depressants, anti-convulsants (e.g., sodium valproate and levetiracetam for myoclonus), secretase inhibitors, amyloid aggregation inhibitors, copper or zinc modulators, BACE inhibitors, inhibitors of tau aggregation, such as Methylene blue, phenothiazines, anthraquinones, n-phenylamines or rhodamines, microtubule stabilizers such as NAP, taxol or paclitaxel, kinase or phosphatase inhibitors such as those targeting GSK30 (lithium) or PP2A, immunization with Aβ peptides or tau phospho-epitopes, anti-tau or anti-amyloid antibodies, dopamine-depleting agents (e.g., tetrabenazine for chorea), benzodiazepines (e.g., clonazepam for myoclonus, chorea, dystonia, rigidity, and/or spasticity), amino acid precursors of dopamine (e.g., levodopa for rigidity), skeletal muscle relaxants (e.g., baclofen, tizanidine for rigidity and/or spasticity), inhibitors for acetylcholine release at the neuromuscular junction to cause muscle paralysis (e.g., botulinum toxin for bruxism and/or dystonia), atypical neuroleptics (e.g., olanzapine and quetiapine for psychosis and/or irritability, risperidone, sulpiride and haloperidol for psychosis, chorea and/or irritability, clozapine for treatment-resistant psychosis, aripiprazole for psychosis with prominent negative symptoms), selective serotonin reuptake inhibitors (SSRIs) (e.g., citalopram, fluoxetine, paroxetine, sertraline, mirtazapine, venlafaxine for depression, anxiety, obsessive compulsive behavior and/or irritability), hypnotics (e.g., xopiclone and/or zolpidem for altered sleep-wake cycle), anticonvulsants (e.g., sodium valproate and carbamazepine for mania or hypomania) and mood stabilizers (e.g., lithium for mania or hypomania).

Neurotrophic factors may be used in combination therapy with the AAV particles described herein for treating neurological disease. Generally, a neurotrophic factor is defined as a substance that promotes survival, growth, differentiation, proliferation and/or maturation of a neuron, or stimulates increased activity of a neuron. In some embodiments, the present methods further comprise delivery of one or more trophic factors into the subject in need of treatment. Trophic factors may include, but are not limited to, IGF-I, GDNF, BDNF, CTNF, VEGF, Colivelin, Xaliproden, Thyrotrophin-releasing hormone and ADNF, and variants thereof.

In one aspect, the AAV particle described herein may be co-administered with AAV particles expressing neurotrophic factors such as AAV-IGF-I (See e.g., Vincent et al., Neuromolecular medicine, 2004, 6, 79-85; the contents of which are incorporated herein by reference in their entirety) and AAV-GDNF (See e.g., Wang et al., J Neurosci., 2002, 22, 6920-6928; the contents of which are incorporated herein by reference in their entirety).

In some embodiments, administration of the AAV particles described herein to a subject will modulate, e.g., increase or decrease, the expression of a target protein in a subject and the modulation, e.g., increase or decrease of the presence, level, activity, and/or expression of the target protein will reduce the effects and/or symptoms of a neurological disease/disorder or a neurodegenerative disorder, a muscular disorder or neuromuscular disorder, and/or a neuro-oncological disorder in a subject.

### Definitions

Unless defined otherwise, all technical and scientific terms used herein have the same meaning as commonly understood by one of ordinary skill in the art to which the invention pertains.

Articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” and “consisting essentially thereof” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

Adeno-associated virus: As used herein, the term “adeno-associated virus” or “AAV” refers to members of the dependovirus genus or a variant, e.g., a functional variant, thereof. In some embodiments, the AAV is wildtype, or naturally occurring. In some embodiments, the AAV is recombinant.

AAV Particle: As used herein, an “AAV particle” refers to a particle or a virion comprising an AAV capsid, e.g., an AAV capsid variant, and a polynucleotide, e.g., a viral genome or a vector genome. In some embodiments, the viral genome of the AAV particle comprises at least one payload region and at least one ITR. In some embodiments, an AAV particle of the disclosure is an AAV particle comprising an AAV variant. In some embodiments, the AAV particle is capable of delivering a nucleic acid, e.g., a payload region, encoding a payload to cells, typically, mammalian, e.g., human, cells. In some embodiments, an AAV particle of the present disclosure may be produced recombinantly. In some embodiments, an AAV particle may be derived from any serotype, described herein or known in the art, including combinations of serotypes (e.g., “pseudotyped” AAV) or from various genomes (e.g., single stranded or self-complementary). In some embodiments, the AAV particle may be replication defective and/or targeted. It is to be understood that reference to the AAV particle of the disclosure also includes pharmaceutical compositions thereof, even if not explicitly recited.

Administering: As used herein, the term “administering” refers to providing a pharmaceutical agent or composition to a subject.

Amelioration: As used herein, the term “amelioration” or “ameliorating” refers to a lessening of severity of at least one indicator of a condition or disease. For example, in the context of neurodegeneration disorder, amelioration comprises the reduction of neuron loss.

Amplicon: As used herein, “amplicon” may refer to any piece of RNA or DNA formed as the product of amplification events, e.g. PCR. In some embodiments, full-length capsid amplicons may be used as templates for next generation sequencing (NGS) library generation. Full-length capsid amplicons may be used for cloning into a DNA library for any number of additional rounds of AAV selection as described herein.

Animal: As used herein, the term “animal” refers to any member of the animal kingdom. In some embodiments, “animal” refers to humans at any stage of development. In some embodiments, “animal” refers to non-human animals at any stage of development. In some embodiments, the non-human animal is a mammal (e.g., a rodent, a mouse, a rat, a rabbit, a monkey, a dog, a cat, a sheep, cattle, a primate, or a pig). In some embodiments, animals comprise, but are not limited to, mammals, birds, reptiles, amphibians, fish, and worms. In some embodiments, the animal is a transgenic animal, genetically-engineered animal, or a clone.

Antisense strand: As used herein, the term “the antisense strand” or “the first strand” or “the guide strand” of a siRNA molecule refers to a strand that is substantially complementary to a section of about 10-50 nucleotides, e.g., about 15-30, 16-25, 18-23 or 19-22 nucleotides of the mRNA of a gene targeted for silencing. The antisense strand or first strand has sequence sufficiently complementary to the desired target mRNA sequence to direct target-specific silencing, e.g., complementarity sufficient to trigger the destruction of the desired target mRNA by the RNAi machinery or process.

Approximately: As used herein, the term “approximately” or “about,” as applied to one or more values of interest, refers to a value that is similar to a stated reference value. As used herein, the term “about” means+/−10% of the recited value. In some embodiments, the term “approximately” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: As used herein, the terms “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization-based connectivity sufficiently stable such that the “associated” entities remain physically associated.

Baculoviral expression vector (BEV): As used herein a BEV is a baculoviral expression vector, e.g., a polynucleotide vector of baculoviral origin. A baculovirus expression vector (BEV) is a recombinant baculovirus that has been genetically modified to lead the expression of a foreign gene. Systems using BEVs are known as baculoviral expression vector systems (BEVSs).

mBEV or modified BEV: As used herein, a modified BEV is an expression vector of baculoviral origin which has been altered from a starting BEV (whether wild type or artificial) by the addition and/or deletion and/or duplication and/or inversion of one or more: genes; gene fragments; cleavage sites; restriction sites; sequence regions; sequence(s) encoding a payload or gene of interest; or combinations of the foregoing.

BIIC: As used herein, a BIIC is a baculoviral infected insect cell.

Capsid: As used herein, the term “capsid” refers to the exterior, e.g., a protein shell, of a virus particle, e.g., an AAV particle, that is substantially (e.g., >50%, >60%, >70%, >80%, >90%, >95%, >99%, or 100%) protein. In some embodiments, the capsid is an AAV capsid comprising an AAV capsid protein described herein, e.g., a VP1, VP2, and/or VP3 polypeptide. The AAV capsid protein can be a wild-type AAV capsid protein or a variant, e.g., a structural and/or functional variant from a wild-type or a reference capsid protein, referred to herein as an “AAV capsid variant.” In some embodiments, the AAV capsid variant described herein has the ability to enclose, e.g., encapsulate, a viral genome and/or is capable of entry into a cell, e.g., a mammalian cell. In some embodiments, the AAV capsid variant described herein may have modified tropism compared to that of a wild-type AAV capsid, e.g., the corresponding wild-type capsid.

Codon optimized: As used herein, the terms “codon optimized” or “codon optimization” refers to a modified nucleic acid sequence which encodes the same amino acid sequence as a parent/reference sequence, but which has been altered such that the codons of the modified nucleic acid sequence are optimized or improved for expression in a particular system (such as a particular species or group of species). As a non-limiting example, a nucleic acid sequence which comprises an AAV capsid protein can be codon optimized for expression in insect cells or in a particular insect cell such Spodopterafrugiperda cells. Codon optimization can be completed using methods and databases known to those in the art.

Complementary and substantially complementary: As used herein, the term “complementary” refers to the ability of polynucleotides to form base pairs with one another. Base pairs are typically formed by hydrogen bonds between nucleotide units in antiparallel polynucleotide strands. Complementary polynucleotide strands can form base pairs in the Watson-Crick manner (e.g., A to T, A to U, C to G), or in any other manner that allows for the formation of duplexes. As persons skilled in the art are aware, when using RNA as opposed to DNA, uracil rather than thymine is the base that is considered to be complementary to adenine. However, when a U is denoted in the context of the present disclosure, the ability to substitute a T is implied, unless otherwise stated. Perfect complementarity or 100% complementarity refers to the situation in which each nucleotide unit of one polynucleotide strand can form a hydrogen bond with a nucleotide unit of a second polynucleotide strand. Less than perfect complementarity refers to the situation in which some, but not all, nucleotide units of two strands can form hydrogen bond with each other. For example, for two 20-mers, if only two base pairs on each strand can form a hydrogen bond with each other, the polynucleotide strands exhibit 10% complementarity. In the same example, if 18 base pairs on each strand can form hydrogen bonds with each other, the polynucleotide strands exhibit 90% complementarity. The term “complementary” as used herein can encompass fully complementary, partially complementary, or substantially complementary. As used herein, the term “substantially complementary” means that the siRNA has a sequence (e.g., in the antisense strand) which is sufficient to bind the desired target mRNA, and to trigger the RNA silencing of the target mRNA. “Fully complementary”, “perfect complementarity”, or “100% complementarity” refers to the situation in which each nucleotide unit of one polynucleotide or oligonucleotide strand can base-pair with a nucleotide unit of a second polynucleotide or oligonucleotide strand.

Conservative amino acid substitution: As used herein, a “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Conserved: As used herein, the term “conserved” refers to nucleotides or amino acid residues of a polynucleotide sequence or polypeptide sequence, respectively, that are those that occur unaltered in the same position of two or more sequences being compared. Nucleotides or amino acids that are relatively conserved are those that are conserved amongst more related sequences than nucleotides or amino acids appearing elsewhere in the sequences. In some embodiments, two or more sequences are said to be “completely conserved” if they are 100% identical to one another. In some embodiments, two or more sequences are said to be “highly conserved” if they are at least 70% identical, at least 80% identical, at least 90% identical, or at least 95% identical to one another. In some embodiments, two or more sequences are said to be “highly conserved” if they are about 70% identical, about 80% identical, about 90% identical, about 95%, about 98%, or about 99% identical to one another. In some embodiments, two or more sequences are said to be “conserved” if they are at least 30% identical, at least 40% identical, at least 50% identical, at least 60% identical, at least 70% identical, at least 80% identical, at least 90% identical, or at least 95% identical to one another. In some embodiments, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence may apply to the entire length of an polynucleotide or polypeptide or may apply to a portion, region or feature thereof.

Control Elements: As used herein, “control elements”, “regulatory control elements” or “regulatory sequences” refers to promoter regions, polyadenylation signals, transcription termination sequences, upstream regulatory domains, origins of replication, internal ribosome entry sites (“IRES”), enhancers, and the like, which provide for the replication, transcription, and translation of a coding sequence in a recipient cell. Not all of these control elements need always be present as long as the selected coding sequence is capable of being replicated, transcribed and/or translated in an appropriate host cell.

Delivery: As used herein, “delivery” refers to the act or manner of delivering an AAV particle, a compound, substance, entity, moiety, cargo, or payload.

Digest: As used herein, the term “digest” means to break apart into smaller pieces or components. When referring to polypeptides or proteins, digestion results in the production of peptides.

Early promoter: An “early promoter,” as used herein, refers a promoter which is recognized by the host cell (e.g., an insect cell, e.g., an Sf9 cell) RNA polymerase II. In some embodiments, an early promoter may comprise a TATA box motif and/or a CAGT motif. In some embodiments, an early promoter is active in a virus (e.g., baculovirus) or a cell infected with the virus before viral DNA replication has occurred. In some embodiments, transcription initiated from an early promoter is alpha amanitin resistant.

Late promoter: A “late promoter,” as used herein, refers to a promoter which is recognized by a viral (e.g., baculovirus) RNA polymerase. In some embodiments, a late promoter may comprise a TAAG motif (e.g., an ATAAG nucleotide sequence).

Early-late promoter: An “early-late promoter,” as used herein, refers a promoter which has components of both an early promoter and a late promoter. In some embodiments, an early-late promoter may comprise a TATA box motif and/or a CAGT motif and a TAAG motif (e.g., an ATAAG nucleotide sequence).

Very late promoter: A “very late promoter,” as used herein, refers a promoter which is recognized by a viral (e.g., baculovirus) RNA polymerase and very late factor 1 (VLF-1). In some embodiments, a very late promoter may comprise a TAAG motif (e.g., an ATAAG nucleotide sequence) and/or a proximal burst element sequence. In some embodiments, VLF-1 binds to the burst element sequence.

Element: As used herein, the term “element” refers to a distinct portion of an entity. In some embodiments, an element may be a polynucleotide sequence with a specific purpose, incorporated into a longer polynucleotide sequence.

Encapsulate: As used herein, the term “encapsulate” means to enclose, surround or encase. As an example, a capsid protein, e.g., an AAV capsid variant, often encapsulates a viral genome. In some embodiments, encapsulate within a capsid, e.g., an AAV capsid variant, encompasses 100% coverage by a capsid, as well as less than 100% coverage, e.g., 95%, 90%, 85%, 80%, 70%, 60% or less. For example, gaps or discontinuities may be present in the capsid so long as the viral genome is retained in the capsid, e.g., prior to entry into a cell.

Engineered: As used herein, embodiments of the present disclosure are “engineered” when they are designed to have a feature or property, whether structural or chemical, that varies from a starting point, wild-type, reference, or native molecule.

Effective Amount: As used herein, the term “effective amount” of an agent is that amount sufficient to effect beneficial or desired results, for example, clinical results, and, as such, an “effective amount” depends upon the context in which it is being applied. For example, in the context of administering an agent that treats cancer, an effective amount of an agent is, for example, an amount sufficient to achieve treatment, as defined herein, of cancer, as compared to the response obtained without administration of the agent.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

ExpressionBac: As used herein, “expressionBac” or “rep/cap bac” refers to a baculovirus comprising an adeno-associated virus (AAV) viral expression construct and/or region. In some embodiments, the viral expression construct of the expressionBac comprises one or more polynucleotides encoding capsid and/or replication genes for an AAV, such as but not limited to AAV2. For example, the one or more polynucleotides encoding capsid and/or replication genes for an AAV may encode VP1, VP2, VP3, Rep52, and/or Rep78, and these polynucleotides may be present in the construct in one or more open reading frames, e.g., in two open reading frames.

Expression BIIC: As used herein, “expression BIIC” or “rep/cap BIIC” refers to an insect cell comprising one or more baculoviruses (e.g., expressionBac) which comprise bacmids comprising a viral expression construct. In some embodiments, the expression construct comprises one or more polynucleotides encoding capsid and/or replication genes for an AAV, such as but not limited to AAV2. For example, the one or more polynucleotides encoding capsid and/or replication genes for an AAV may encode VP1, VP2, VP3, Rep52, and/or Rep78, and these polynucleotides may be present in the construct in one or more open reading frames, e.g., in two open reading frames. In some embodiments, the insect cell is an Sf9 cell.

Formulation: As used herein, a “formulation” comprises at least one AAV particle and a delivery agent or excipient.

Fragment: A “fragment,” as used herein, refers to a portion. For example, an antibody fragment may comprise a CDR, or a heavy chain variable region, or a scFv, etc.

Functional: As used herein, a “functional” biological molecule is a biological molecule in a form in which it exhibits a property and/or activity by which it is characterized.

Gene expression: The term “gene expression” refers to the process by which a nucleic acid sequence undergoes successful transcription and in most instances translation to produce a protein or peptide. For clarity, when reference is made to measurement of “gene expression”, this should be understood to mean that measurements may be of the nucleic acid product of transcription, e.g., RNA or mRNA or of the amino acid product of translation, e.g., polypeptides or peptides. Methods of measuring the amount or levels of RNA, mRNA, polypeptides and peptides are well known in the art.

Homology: As used herein, the term “homology” refers to the overall relatedness between polymeric molecules, e.g. between polynucleotide molecules (e.g. DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). In accordance with the disclosure, two polynucleotide sequences are considered to be homologous if the polypeptides they encode are at least about 50%, 60%, 70%, 80%, 90%, 95%, or even 99% for at least one stretch of at least about 20 amino acids. In some embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. In accordance with the disclosure, two protein sequences are considered to be homologous if the proteins are at least about 50%, 60%, 70%, 80%, or 90% identical for at least one stretch of at least about 20 amino acids.

Identity: As used herein, the term “identity” refers to the overall relatedness between polymeric molecules, e.g., between polynucleotide molecules (e.g. DNA molecules and/or RNA molecules) and/or between polypeptide molecules. Calculation of the percent identity of two polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In some embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; the contents of each of which are incorporated herein by reference in their entirety. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

Inhibit expression of a gene: As used herein, the phrase “inhibit expression of a gene” means to cause a reduction in the amount of an expression product of the gene. The expression product can be an RNA transcribed from the gene (e.g., an mRNA) or a polypeptide translated from an mRNA transcribed from the gene. Typically, a reduction in the level of an mRNA results in a reduction in the level of a polypeptide translated therefrom. The level of expression may be determined using standard techniques for measuring mRNA or protein.

Inverted terminal repeat: As used herein, the term “inverted terminal repeat” or “ITR” refers to a cis-regulatory element for the packaging of polynucleotide sequences into viral capsids.

Isolated: As used herein, the term “isolated” refers to a substance or entity that is altered or removed from the natural state, e.g., altered or removed from at least some of component with which it is associated in the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell. Such polynucleotides could be part of a vector and/or such polynucleotides or polypeptides could be part of a composition, and still be isolated in that such vector or composition is not part of the environment in which it is found in nature. In some embodiments, an isolated nucleic acid is recombinant, e.g., incorporated into a vector.

Junction: As used herein, the term “junction” refers to a double stranded nucleotide sequence that overlaps by, e.g., 1-50 base pairs, between two adjacent regions (e.g., a subgenomic region, a subregion, a subfragment, a fragment, or subgenomic fragment), wherein the 3′ end of a first region is base paired to the 5′ end of the second region. In some embodiments, the junctions are unique, e.g., a unique junction.

Unique Junction: As used herein, the term “unique junction” refers to a junction that is present once in the baculovirus expression construct or baculovirus genome (e.g., variant baculovirus genome).

Linker: As used herein “linker” refers to a molecule or group of molecules which connects two molecules. A linker may be a nucleic acid sequence connecting two nucleic acid sequences encoding two different polypeptides. The linker may or may not be translated. The linker may be a cleavable linker.

MicroRNA (miRNA) binding site: As used herein, a microRNA (miRNA) binding site represents a nucleotide location or region of a nucleic acid transcript to which at least the “seed” region of a miRNA binds.

Modified: As used herein, “modified” refers to a changed state or structure of a molecule of the present disclosure. Molecules may be modified in many ways comprising chemically, structurally, and functionally. As used herein, embodiments of the disclosure are “modified” when they have or possess a feature or property, whether structural or chemical, that varies from a starting point, wild type or native molecule.

Mutation: As used herein, the term “mutation” refers to any changing of the structure of a gene, resulting in a variant (also called “mutant”) form that may be transmitted to subsequent generations. Mutations in a gene may be caused by the alternation of single base in DNA, or the deletion, insertion, or rearrangement of larger sections of genes or chromosomes.

Non-essential gene: As used herein, the term “non-essential gene” when used in the context of baculovirus refers to a gene or gene regulatory region that, when modified, does not substantially alter, e.g., reduce, the amount of infectious baculovirus produced during infection. In some embodiments, modification of a non-essential gene does not reduce very late gene transcription. In some embodiments, modifications to non-essential genes or regulatory regions of non-essential genes include any alteration that results in a reduction or elimination of a gene product, e.g., deletion, insertion, mutation (e.g., frame-shift mutations), promoter modification, or inserting a heterologous DNA adjacent to a non-essential gene.

Nucleic Acid: As used herein, the term “nucleic acid”, “polynucleotide” and ‘oligonucleotide” refer to any nucleic acid polymers composed of either polydeoxyribonucleotides (containing 2-deoxy-D-ribose), or polyribonucleotides (containing D-ribose), or any other type of polynucleotide which is an N glycoside of a purine or pyrimidine base, or modified purine or pyrimidine bases. There is no intended distinction in length between the term “nucleic acid”, “polynucleotide” and “oligonucleotide”, and these terms will be used interchangeably. These terms refer only to the primary structure of the molecule. Thus, these terms comprise double- and single-stranded DNA, as well as double- and single stranded RNA.

Open reading frame: As used herein, “open reading frame” or “ORF” refers to a sequence which does not contain a stop codon within the given reading frame, other than at the end of the reading frame.

Operably linked: As used herein, the phrase “operably linked” refers to a functional connection between two or more molecules, constructs, transcripts, entities, moieties, or the like. As a non-limiting example, a promoter is “operably linked” to a nucleotide sequence when the promoter sequence controls and/or regulates the transcription of the nucleotide sequence.

5′ overhang: As used herein, the term “5′ overhang” refers to a single stranded cohesive end located at the 5′ end (e.g., left end) of a fragment (e.g., subgenomic fragment or subfragment) or a linearized vector (e.g., destination vector).

3′ overhang: As used herein, the term “3′ overhang” refers to a single stranded cohesive end located at the 3′ end (e.g., right end) of a fragment (e.g., subgenomic fragment or subfragment) or a linearized vector (e.g., destination vector).

Cohesive end: As used herein, the term “cohesive end” in reference to a 5′ overhang or 3′ overhang refers to a single stranded nucleotide sequence at the 5′ end of a fragment that is complementary to an overhang at the 3′ end of an adjacent fragment, or a single stranded nucleotide sequence at the 3′ end of a fragment that is complementary to an overhang at the 5′ end of an adjacent fragment. In some embodiments, a cohesive end refers to a single stranded nucleotide sequence at the 5′ end of a linearized vector (e.g., a destination vector, e.g., a destination bacmid) that is complementary to an overhang at the 3′ end of a fragment, or a single stranded nucleotide sequence at the 3′ end of a linearized vector (e.g., destination vector, e.g., a destination bacmid) that is complementary to an overhang at the 5′ end of a adjacent fragment.

Payload region: As used herein, a “payload region” is any nucleic acid sequence (e.g., within the viral genome) which encodes one or more “payloads” of the disclosure. As non-limiting examples, a payload region may be a nucleic acid sequence within the viral genome of an AAV particle, which encodes a payload, wherein the payload is an RNAi agent or a polypeptide. Payloads of the present disclosure may be, but are not limited to, peptides, polypeptides, proteins, antibodies, RNAi agents, etc.

PayloadBac: As used herein, “payloadBac” refers to a baculovirus comprising a payload construct and/or region. In some embodiments, the payload construct and/or region of the payloadBac comprises a polynucleotide encoding the payload.

Payload BIIC: As used herein, “payloadBIIC” refers to an insect cell comprising one or more baculovirus (e.g., payloadBac) comprising a payload construct and/or region. In some embodiments, the payload construct and/or region comprises a polynucleotide encoding the payload. In some embodiments, the insect cell is an Sf9 cell.

Payload construct: As used herein, “payload construct” is one or more vector construct which comprises a polynucleotide region encoding or comprising a payload that is flanked on one or both sides by an inverted terminal repeat (ITR) sequence. The payload construct may present a template that is replicated in a viral production cell to produce a therapeutic viral genome.

Payload construct vector: As used herein, “payload construct vector” is a vector encoding or comprising a payload construct, and regulatory regions for replication and expression of the payload construct in bacterial cells.

Payload construct expression vector: As used herein, a “payload construct expression vector” is a vector encoding or comprising a payload construct and which further comprises one or more polynucleotide regions encoding or comprising components for viral expression in a viral replication cell.

Polypeptide: As used herein, “polypeptide” means a polymer of amino acid residues (natural or unnatural) linked together most often by peptide bonds. The term, as used herein, refers to proteins, polypeptides, and peptides of any size, structure, or function. In some instances, the polypeptide encoded is smaller than about 50 amino acids and the polypeptide is then termed a peptide. If the polypeptide is a peptide, it will be at least about 2, 3, 4, or at least 5 amino acid residues long. Thus, polypeptides include gene products, naturally occurring polypeptides, synthetic polypeptides, homologs, orthologs, paralogs, fragments and other equivalents, variants, and analogs of the foregoing. A polypeptide may be a single molecule or may be a multi-molecular complex such as a dimer, trimer or tetramer. They may also comprise single chain or multichain polypeptides and may be associated or linked. The term polypeptide may also apply to amino acid polymers in which one or more amino acid residues are an artificial chemical analogue of a corresponding naturally occurring amino acid.

Polypeptide variant: The term “polypeptide variant” refers to molecules which differ in their amino acid sequence from a native or reference sequence. The amino acid sequence variants may possess substitutions, deletions, and/or insertions at certain positions within the amino acid sequence, as compared to a native or reference sequence. In some embodiments, a variant comprises a sequence having at least about 50%, at least about 80%, or at least about 90%, identical (homologous) to a native or a reference sequence.

Peptide: As used herein, “peptide” is less than or equal to 50 amino acids long, e.g., about 5, 10, 15, 20, 25, 30, 35, 40, 45, or 50 amino acids long.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Preventing: As used herein, the term “preventing” or “prevention” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

Prophylactic: As used herein, “prophylactic” refers to a therapeutic or course of action used to prevent the spread of disease.

Prophylaxis: As used herein, a “prophylaxis” refers to a measure taken to maintain health and prevent the spread of disease.

Region: As used herein, the term “region” refers to a zone or general area. In some embodiments, when referring to a protein or protein module, a region may comprise a linear sequence of amino acids along the protein or protein module or may comprise a three-dimensional area, an epitope and/or a cluster of epitopes. In some embodiments, regions comprise terminal regions. As used herein, the term “terminal region” refers to regions located at the ends or termini of a given agent. When referring to proteins, terminal regions may comprise N- and/or C-termini.

In some embodiments, when referring to a polynucleotide, a region may comprise a linear sequence of nucleic acids along the polynucleotide or may comprise a three-dimensional area, secondary structure, or tertiary structure. In some embodiments, regions comprise terminal regions. As used herein, the term “terminal region” refers to regions located at the ends or termini of a given agent. When referring to polynucleotides, terminal regions may comprise 5′ and/or 3′ termini.

RNA or RNA molecule: As used herein, the term “RNA” or “RNA molecule” or “ribonucleic acid molecule” refers to a polymer of ribonucleotides; the term “DNA” or “DNA molecule” or “deoxyribonucleic acid molecule” refers to a polymer of deoxyribonucleotides. DNA and RNA can be synthesized naturally, e.g., by DNA replication and transcription of DNA, respectively; or be chemically synthesized. DNA and RNA can be single-stranded (i.e., ssRNA or ssDNA, respectively) or multi-stranded (e.g., double stranded, i.e., dsRNA and dsDNA, respectively). The term “mRNA” or “messenger RNA”, as used herein, refers to a single stranded RNA that encodes the amino acid sequence of one or more polypeptide chains.

RNA interfering or RNAi: As used herein, the term “RNA interfering” or “RNAi” refers to a sequence specific regulatory mechanism mediated by RNA molecules which results in the inhibition or interfering or “silencing” of the expression of a corresponding protein-coding gene. RNAi has been observed in many types of organisms, including plants, animals and fungi. RNAi occurs in cells naturally to remove foreign RNAs (e.g., viral RNAs). Natural RNAi proceeds via fragments cleaved from free dsRNA which direct the degradative mechanism to other similar RNA sequences. RNAi is controlled by the RNA-induced silencing complex (RISC) and is initiated by short/small dsRNA molecules in cell cytoplasm, where they interact with the catalytic RISC component argonaute. The dsRNA molecules can be introduced into cells exogenously. Exogenous dsRNA initiates RNAi by activating the ribonuclease protein Dicer, which binds and cleaves dsRNAs to produce double-stranded fragments of 21-25 base pairs with a few unpaired overhang bases on each end. These short double stranded fragments are called small interfering RNAs (siRNAs).

RNAi agent: As used herein, the term “RNAi agent” refers to an RNA molecule, or its derivative, that can induce inhibition, interfering, or “silencing” of the expression of a target gene and/or its protein product. An RNAi agent may knock-out (virtually eliminate or eliminate) expression, or knock-down (lessen or decrease) expression. The RNAi agent may be, but is not limited to, dsRNA, siRNA, shRNA, pre-miRNA, pri-miRNA, miRNA, stRNA, lncRNA, piRNA, or snoRNA.

miR binding site: As used herein, a “miR binding site” comprises a nucleic acid sequence (whether RNA or DNA, e.g., differ by “U” of RNA or “T” in DNA) that is capable of binding, or binds, in whole or in part to a microRNA (miR) through complete or partial hybridization. Typically, such binding occurs between the miR and the miR binding site in the reverse complement orientation. In some embodiments, the miR binding site is transcribed from the AAV vector genome encoding the miR binding site.

In some embodiments, a miR binding site may be encoded or transcribed in series. Such a “miR binding site series” or “miR BSs” may include two or more miR binding sites having the same or different nucleic acid sequence.

Sample: As used herein, the term “sample” or “biological sample” refers to a subset of its tissues, cells, nucleic acids, or component parts (e.g. body fluids, including but not limited to blood, serum, mucus, lymphatic fluid, synovial fluid, cerebrospinal fluid, saliva, amniotic fluid, amniotic cord blood, urine, vaginal fluid and semen).

Self-complementary viral particle: As used herein, a “self-complementary viral particle” is a particle comprised of at least two components, a protein capsid and a polynucleotide sequence encoding a self-complementary genome enclosed within the capsid.

Sense Strand: As used herein, the term “the sense strand” or “the second strand” or “the passenger strand” of a siRNA molecule refers to a strand that is complementary to the antisense strand or first strand. The antisense and sense strands of a siRNA molecule are hybridized to form a duplex structure. As used herein, a “siRNA duplex” includes a siRNA strand having sufficient complementarity to a section of about 10-50 nucleotides of the mRNA of the gene targeted for silencing and a siRNA strand having sufficient complementarity to form a duplex with the other siRNA strand.

Similarity: As used herein, the term “similarity” refers to the overall relatedness between polymeric molecules, e.g. between polynucleotide molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art.

Short interfering RNA or siRNA: As used herein, the terms “short interfering RNA,” “small interfering RNA” or “siRNA” refer to an RNA molecule (or RNA analog) comprising between about 5-60 nucleotides (or nucleotide analogs) which is capable of directing or mediating RNAi. Preferably, a siRNA molecule comprises between about 15-30 nucleotides or nucleotide analogs, such as between about 16-25 nucleotides (or nucleotide analogs), between about 18-23 nucleotides (or nucleotide analogs), between about 19-22 nucleotides (or nucleotide analogs) (e.g., 19, 20, 21 or 22 nucleotides or nucleotide analogs), between about 19-25 nucleotides (or nucleotide analogs), and between about 19-24 nucleotides (or nucleotide analogs). The term “short” siRNA refers to a siRNA comprising 5-23 nucleotides, preferably 21 nucleotides (or nucleotide analogs), for example, 19, 20, 21 or 22 nucleotides. The term “long” siRNA refers to a siRNA comprising 24-60 nucleotides, preferably about 24-25 nucleotides, for example, 23, 24, 25 or 26 nucleotides. Short siRNAs may, in some instances, include fewer than 19 nucleotides, e.g., 16, 17 or 18 nucleotides, or as few as 5 nucleotides, provided that the shorter siRNA retains the ability to mediate RNAi. Likewise, long siRNAs may, in some instances, include more than 26 nucleotides, e.g., 27, 28, 29, 30, 35, 40, 45, 50, 55, or even 60 nucleotides, provided that the longer siRNA retains the ability to mediate RNAi or translational repression absent further processing, e.g., enzymatic processing, to a short siRNA. siRNAs can be single stranded RNA molecules (ss-siRNAs) or double stranded RNA molecules (ds-siRNAs) comprising a sense strand and an antisense strand which hybridized to form a duplex structure called an siRNA duplex.

Signal Sequences: As used herein, the phrase “signal sequences” refers to a sequence which can direct the transport or localization of a protein.

Similarity: As used herein, the term “similarity” refers to the overall relatedness between polymeric molecules, e.g., between polynucleotide molecules (e.g., DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art.

Spacer: As used here, a “spacer” is generally any selected nucleic acid sequence of, e.g., 1, 2, 3, 4, 5, 6, 7, 8, 9, or 10 nucleotides in length, which is located between two or more consecutive miR binding site sequences. Spacers may also be more than 10 nucleotides in length, e.g., 20, 30, 40, or 50 or more than 50 nucleotides.

Subject: As used herein, the term “subject” or “patient” refers to any organism to which a composition in accordance with the present disclosure may be administered, e.g., for experimental, diagnostic, prophylactic, and/or therapeutic purposes. Typical subjects comprise animals (e.g., mammals such as mice, rats, rabbits, non-human primates, and humans) and/or plants. The subject or patient may seek or need treatment, require treatment, is receiving treatment, will receive treatment, or is under care by a trained professional for a particular disease or condition.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Synthetic or chemically synthesized: As used herein, the term “synthetic” or “chemically synthesized” in the context of a nucleic acid sequence refers to a nucleic acid molecule that is, at least in part, formed through a chemical process, as opposed to molecules of natural origin, or molecules derived via template-based amplification of molecules of natural origin. In some embodiments, chemically-synthesized DNA is non-templated (e.g., the sequence is arbitrarily decided and does not physically depend on a parental sequence), unlike natural DNA replication or in vitro polymerase reactions like PCR.

Target Cells: As used herein, “target cells” or “target tissue” refers to any one or more cells of interest. The cells may be found in vitro, in vivo, in situ or in the tissue or organ of an organism. The organism may be an animal, preferably a mammal, more preferably a human and most preferably a patient.

Therapeutic Agent: The term “therapeutic agent” refers to any agent that, when administered to a subject, has a therapeutic, diagnostic, and/or prophylactic effect and/or elicits a desired biological and/or pharmacological effect.

Therapeutically effective amount: As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition. In some embodiments, a therapeutically effective amount is provided in a single dose.

Therapeutically effective outcome: As used herein, the term “therapeutically effective outcome” means an outcome that is sufficient in a subject suffering from or susceptible to an infection, disease, disorder, and/or condition, to treat, improve symptoms of, diagnose, prevent, and/or delay the onset of the infection, disease, disorder, and/or condition.

Transfection: As used herein, the term “transfection” refers to methods to introduce exogenous nucleic acids into a cell. Methods of transfection comprise, but are not limited to, chemical methods, physical treatments and cationic lipids or mixtures.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Unique junction: As used herein, the term “unique junction” refers to a junction formed by the covalent linkage of a unique 5′ overhang in one fragment with a unique 3′ overhang in another fragment based on complementarity of the overhangs. A “unique junction” may also be formed by the covalent linkage of a unique 5′ overhang in a fragment and the unique 3′ overhang of a destination vector (e.g., a carrier vector or baculovirus expression construct), or the unique 3′ overhang in a fragment and the unique 5′ overhang of a destination vector.

Unique overhang: As used herein, a unique overhang refers to a 5′ or 3′ overhang of a fragment that only pairs with a single other 3′ or 5′ overhang, respectively, of another fragment within a plurality of fragments. In some embodiments, a unique 5′ overhang of a subgenomic fragment is a 5′ overhang that only pairs with a single 3′ overhang of another subgenomic fragment, or the 3′ overhang of a destination vector (e.g., a carrier vector or baculovirus expression construct). Similarly, in some embodiments, a unique 3′ overhang of a subgenomic fragment is a 3′ overhang that only pairs with a single 5′ overhang of another subgenomic fragment, or the 5′ overhang of a destination vector.

Unmodified: As used herein, “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification. Molecules (e.g., a baculovirus genome) may also be unmodified with respect to a reference molecule (e.g., a reference baculovirus genome).

Variant: As used herein, the term “variant” refers to a polypeptide or polynucleotide that has an amino acid or a nucleotide sequence that is substantially identical, e.g., having at least 70%, 75%, 80%, 85%, 90%, 95% or 99% sequence identity to a reference sequence. In some embodiments, the variant is a functional variant. As used herein, the term “functional variant” refers to a polypeptide variant or a polynucleotide variant that has at least one activity of the reference sequence. “Insertional variants” when referring to polypeptides are those with one or more amino acids inserted, e.g., immediately adjacent or subsequent, to a position in an amino acid sequence. “Immediately adjacent” or “immediately subsequent” to an amino acid means connected to either the alpha-carboxy or alpha-amino functional group of the amino acid. “Deletional variants” when referring to polypeptides, are those with one or more amino acids in deleted from a reference protein.

Vector: As used herein, the term “vector” refers to any molecule or moiety which transports, transduces or otherwise acts as a carrier of a heterologous molecule. In some embodiments, vectors may be plasmids. In some embodiments, vectors may be viruses. An AAV particle is an example of a vector. Vectors of the present disclosure may be produced recombinantly and may be based on and/or may comprise adeno-associated virus (AAV) parent or reference sequences. The heterologous molecule may be a polynucleotide and/or a polypeptide.

Viral Genome: As used herein, the terms “viral genome” or “vector genome”, when used in the context of AAV, refers to the nucleic acid sequence(s) encapsulated in an AAV particle. A viral genome comprises a nucleic acid sequence with at least one payload region encoding a payload and at least one ITR.

### Equivalents and Scone

The disclosures of each and every patent, patent application, publication, and sequences cited herein are hereby incorporated herein by reference in their entirety. Those skilled in the art will recognize or be able to ascertain using no more than routine experimentation, many equivalents to the specific embodiments in accordance with the disclosure described herein. The scope of the present disclosure is not intended to be limited to the above Description, but rather is as set forth in the appended claims.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the disclosure (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the disclosure.

The present disclosure is further illustrated by the following non-limiting examples.

### EXAMPLES

### Example 1: Generation of Synthetic Baculovirus Vector Construct

This Example describes the generation of a baculovirus vector construct containing a synthetic baculovirus genome by assembling several nucleic acid molecules into a single contiguous sequence using the Golden Gate Assembly strategy. The strategy avoids the need for step-by-step modification of previous baculovirus designs, which is both labor intensive, inefficient, and prone to errors. Instead, multiple independently designed improvements to a reference (e.g., parental) baculovirus vector can be introduced simultaneously by introducing the changes into subgenomic plasmids containing the improvements and combining fragments released from the plasmids to build an entirely new baculovirus genome de novo.

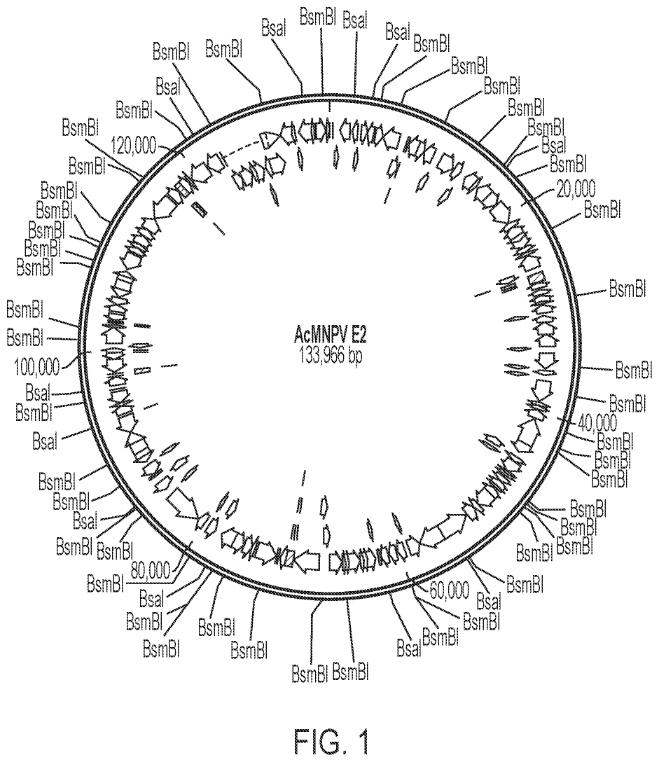

As an design initial step, all BsaI and BsmBI restriction enzyme recognition sites (both are Type IIS restriction enzymes) in a baculovirus genome (accession KM667940.1; wild-type AcMNPV, strain E2) were removed by introducing silent mutations (i.e., mutations which do not alter the amino acid sequence of a gene when the Type IIS site is located within a gene coding sequence). This process is referred to as domestication of the baculovirus genome. There are a total of 11 BsaI sites and 44 BsmBI sites in the wild-type genome of AcMNPV strain E2 (accession KM667940.1) (FIG. 1). With regard to other Type IIS restriction sites which can be used with this strategy, 5 AarI recognition sites are also present in the wild-type AcMNPV strain E2 genome.

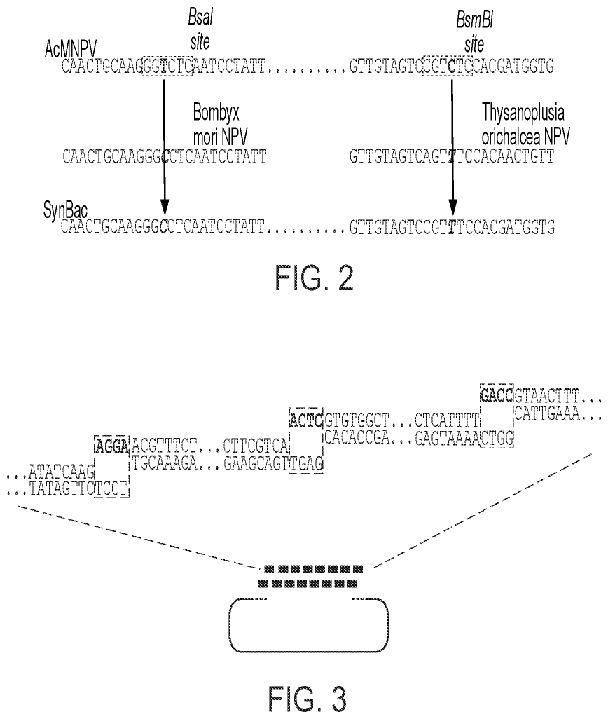

To minimize the possibility of impacting an essential baculovirus function by introducing mutations that abolish a Type IIS restriction site, mutations were derived from variants found in other alpha-baculovirus isolates in the NCBI database, for example, as shown in FIG. 2.

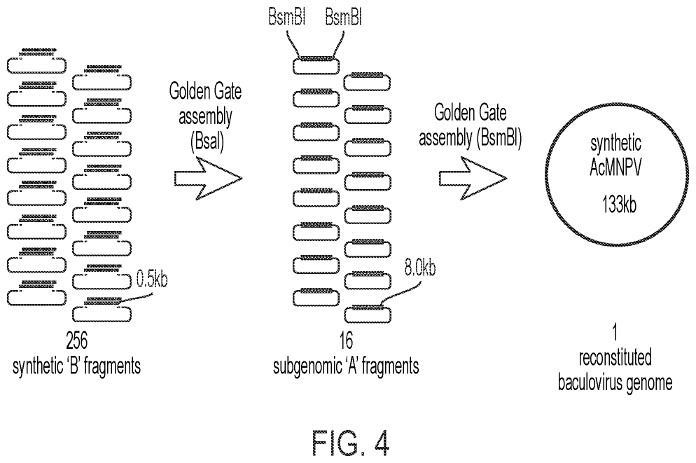

Next, a software algorithm was used to partition the input sequence (i.e., the parental baculovirus genome sequence lacking the BsaI and BsmBI sites) into a specified number of subgenomic ‘A’ fragments. The ‘A’ fragments were then partitioned (using the same algorithm) into a desired number of smaller synthetic ‘B’ fragments. In this example, the parental baculovirus vector was partitioned into 16 subgenomic ‘A’ fragments (about 8 kb each), and each ‘A’ fragment was assembled from 16 synthetic ‘B’ fragments (about 0.5 kb each; total of 256 synthetic ‘B’ fragments to assemble a baculoviral genome). Briefly, the algorithm defines a class JUNCTION, which is a region of the input sequence of a given nucleotide window size. The algorithm then assigns a single 4-base overhang to a set of JUNCTION objects such that they are compatible with each other in Golden Gate Assembly. FIGS. 3 and 4 show schematics of how the junctions allow the contiguous ordered assembly of the 16 subgenomic ‘A’ fragments, ultimately resulting in the assembly of a full synthetic baculovirus genome. The same underlying principle applies for how each subgenomic ‘A’ fragment is assembled from the synthetic ‘B’ fragments.

The 256 ‘B’ fragments together with the flanking BsaI sites were chemically synthesized and cloned into plasmids. The BsaI and BsmBI recognition sites within the genome were abolished by incorporating the mutations (designed at the in silico stage) directly into the ‘B’ fragments as they were being chemically synthesized. Sequences to be synthesized had the following configuration: 5′-NNN(BsaI site→NXXXX[‘B’ fragment sequence]XXXXN(←BsaI site)NNN-3′, wherein Ns correspond to stuffer nucleotides, and XXXX are the intended terminal 4-base overhangs at the ends of the ‘B’ fragment left after cutting with BsaI. Once chemically synthesized, the ‘B’ fragments were cloned individually into standard cloning plasmids which lack recognition sites for the type IIS restriction enzyme used in this step (BsaI in this example).

The plasmids containing ‘B’ fragments that constituted a subgenomic ‘A’ fragment were combined, digested with BsaI, and ligated into a destination vector via Golden Gate assembly reaction. The unique overhangs allowed for ligation of the fragments in the desired order into a subgenomic ‘A’ fragment. Sixteen separate reactions were performed to form the 16 subgenomic ‘A’ fragments, which were captured in Golden Gate destination vectors. These destination vectors were designed such a LacZa reporter for blue/white colony screening would be removed during BsaI Golden Gate assembly and replaced with the assembled ‘A’ fragment. Furthermore, these destination vectors also included a BsmBI recognition site that flanked each end of the ‘A’ fragment insert, which enables the subsequent Golden Assembly of ‘A’ fragments into the complete baculovirus genome. Briefly, equimolar amounts (100 fmol) of each ‘B’ fragment-containing plasmid was combined in a one-pot reaction with (a) a destination vector (50 fmol), (b) T4 ligase buffer/10 mM ATP, (c) BsaI enzyme (30 U), and (d) T4 ligase enzyme (2500 U) in a 25 ul reaction. The mixtures were subjected to (a) 60 cycles of 37° C. for 5 minutes, followed by 16° C. for 5 minutes, and (b) 1 cycle of 60° C., followed by holding at 4° C.

Once the 16 subgenomic ‘A’ fragment-containing plasmids were prepared, in a second Golden Gate assembly the fragments were released from the plasmids by digestion with BsmBI and allowed to assemble into a synthetic baculovirus genome via the unique overhangs in a ligation reaction using DNA ligase. In this example, the complete genome is captured in a Golden Gate-compatible destination vector, such as a domesticated bacterial artificial chromosome (BAC). Briefly, equimolar amounts (100 fmol) of each ‘A’ fragment-containing plasmid was combined in a one-pot reaction with (a) a destination vector (50 fmol), (b) T4 ligase buffer/10 mM ATP, (c) BsmBI enzyme (30 U), and (d) T4 ligase enzyme (2500 U) in a 25 ul reaction. The mixtures were subjected to (a) 60 cycles of 42° C. for 50 minutes, followed by 16° C. for 5 minutes, and (b) 1 cycle of 60° C., followed by holding at 4° C.

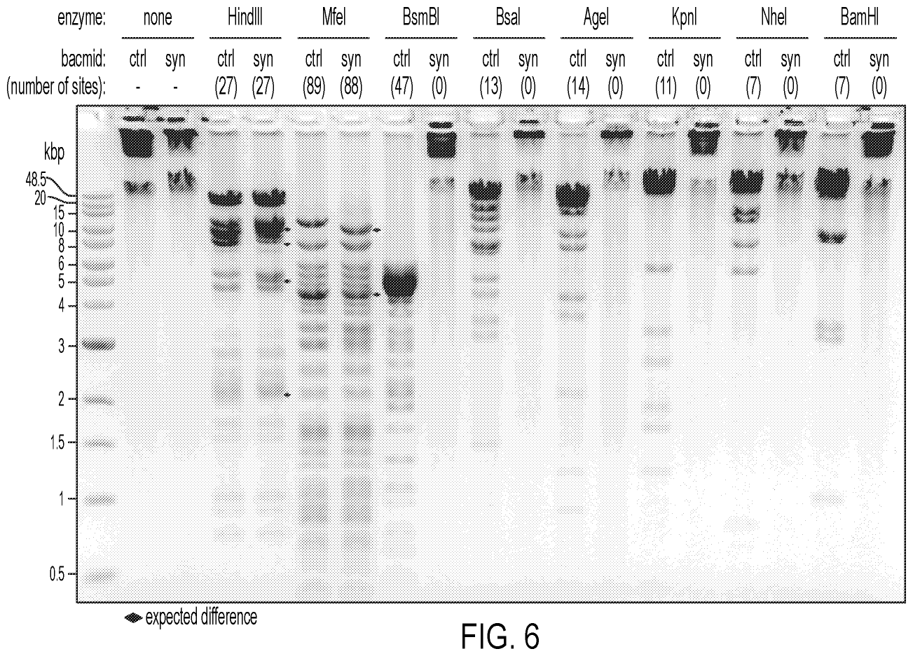

The fully assembled baculovirus genome was then ligated into a bacterial artificial chromosome (BAC) to maintain the final construct in E. coli, using the outermost overhang sequences ACAA (shown in FIG. 5) and CATC (not shown in FIG. 5, but corresponding to the end overhang of the ‘A’ fragment that begins with the TACA overhang, which is shown in FIG. 5). This resulted in the replacement of part of the native polyhedrin locus with the BAC sequence.

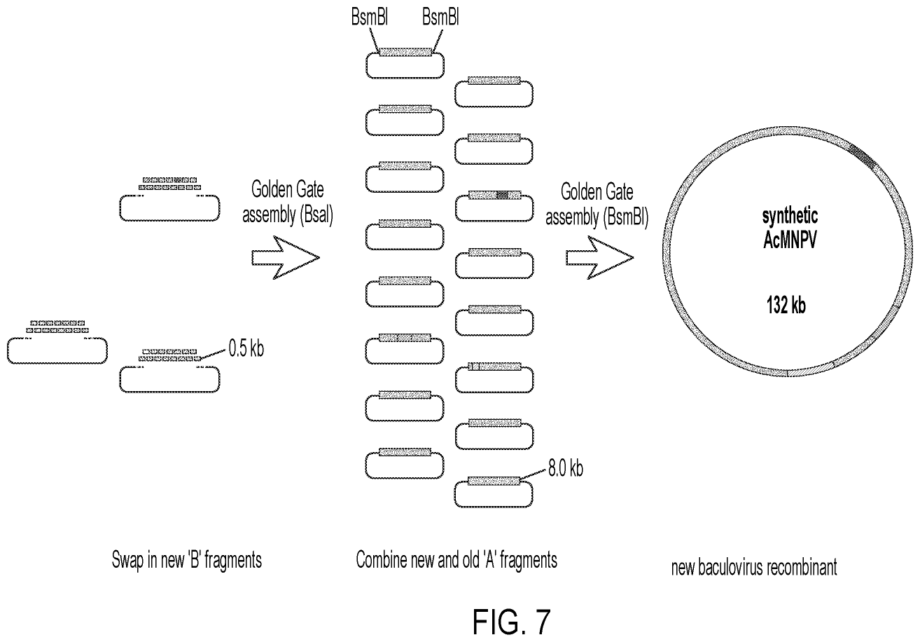

To demonstrate that the synthetic baculovirus construct is derived from non-templated, synthetic DNA, a bacmid containing the fully assembled synthetic baculovirus genome using the above strategy was digested with various enzymes having the same or different number of recognition sites relative to a control bacmid containing the wild-type AcMNPV strain E2 genome with the exception of removal of the polyhedrin locus. As shown in FIG. 6, the HindIII enzyme, which has the same number of recognition sites in the control and synthetic genomes showed the same digestion pattern for both bacmids, whereas different patterns were observed for enzymes having a different number of recognition sites.

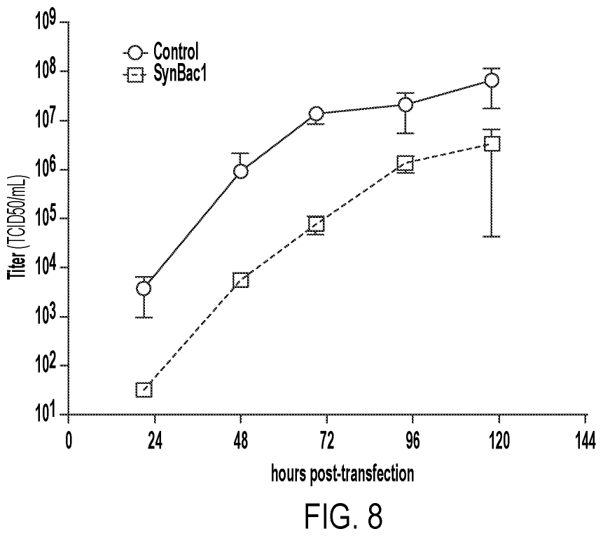

The above synthetic baculovirus genome generation strategy also can be used to manipulate the baculovirus genome, for example, to introduce multiple modifications such as substitutions, additions, deletions, or even introduction of transgene sequences of interest (viral genes) for expression, in different parts of the genome. This can be achieved by first determining the loci where the modification(s) are desired, identifying the corresponding synthetic ‘B’ fragments that include the loci, making the modification(s) using standard cloning techniques (e.g., site-directed mutagenesis), and assembling the baculovirus genome using the procedure described above. FIG. 7 shows a schematic of how such modifications can be made to generate a variant baculovirus genome.

### Example 2: Introducing Modifications in the Synthetic Baculovirus Genome

This Example describes how a synthetic baculovirus genome prepared using the NCBI genome of accession KM667940.1 as starting material was modified based on the method described in Example 1.

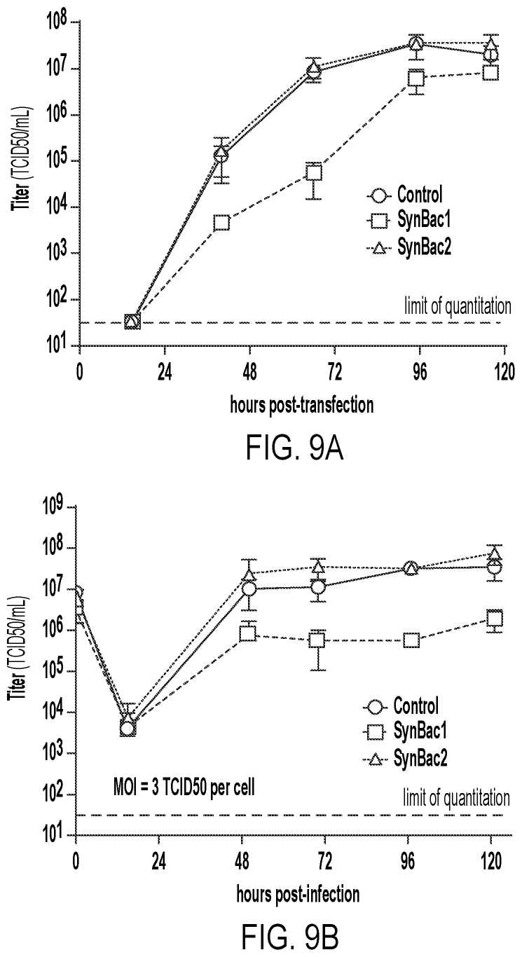

Baculovirus produced from the synthetic baculovirus genome-containing bacmid generated in Example 1 (SynBac1) was replication competent, but exhibited a replication defect, as evidenced by a lower titer relative to baculovirus produced from a Control bacmid (“Control”; bMON14272 (Luckow et al., J Virology 1993; 67:4566-79) containing the AcMNPV-E2 genome, except for the polyhedrin locus), as shown in FIG. 8. Re-sequencing of wild-type AcMNPV E2 DNA and comparison of sequencing reads to KM667940.1 revealed that KM667940.1 contains insertion errors in genes lef8 and p49. The replication defect in SynBac1 was attributed to the incorporation of these insertion errors.

A new synthetic baculovirus genome with wild-type p49 and lef8 protein sequences was therefore constructed as follows. Plasmids containing the relevant synthetic ‘B’ fragments having the insertional errors in p49 and lef8 were identified. Corrected ‘B’ fragments containing the true wild-type sequence were newly chemically synthesized, subgenomic ‘A’ fragments were assembled using the corrected ‘B’ fragments, and the entire synthetic baculovirus genome was assembled using the corrected subgenomic A′ fragments and cloned into a bacmid as described in Example 1.

Baculovirus produced from the corrected synthetic bacmid (SynBac2) was then tested for replication activity. Briefly, 5 μg of purified Control, SynBac1, and SynBac2 bacmids were each combined in triplicate with 200 μL of Grace's Salts and 10 μL of Mirus TransIT Insect Cell transfection reagent to generate transfection complexes. After 20 min incubation at room temperature, transfection complexes were added dropwise to Sf9 shake flask cultures (30 mL at 1.2×10⁶ viable cells per mL). Cultures were shaken at 140 RPM at 28° C. and samples were taken at the indicated time-points. The cell supernatants were harvested after five days as budded virus stocks. For single-cycle replication studies, Sf9 shake flask cultures (as above) were infected at a multiplicity-of-infection (MOI) of 3 Tissue-Culture-Infectious-Dose-50% (TCID50) units per cell using the budded virus stocks above. Samples were taken at the indicated time-points.

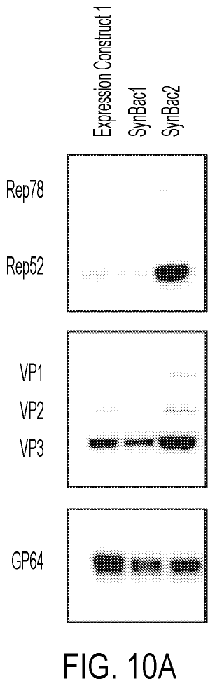

As shown in FIGS. 9A and 9B, baculovirus produced from SynBac2 showed replication which was comparable to baculovirus produced from the Control both after transfection of Sf9 cells (FIG. 9A) and after infection of Sf9 cells with an MOI 3 TCID50 per cell (FIG. 9B). These results demonstrate that a replication competent AcMNPV genome (˜130 kb) can be assembled entirely from chemically-synthesized DNA sequences, and that fast and reliable manipulation (e.g., correction) of sequences can be achieved.

### Example 3: Production of rAAV1 Using Synthetic Baculovirus Genome

This Example describes the production of rAAV1 using a synthetic baculovirus genome (constructed as described and tested above in Example 2) into which adeno-associated virus (AAV) genes were added.

Briefly, Rep52 and Rep78/AAV1 capsid genes were PCR amplified and cloned into Synbac2 digested with AgeI and AvrII enzymes by Gibson Assembly™ reaction to create the Synbac2-AAV1/Rep bacmid. The assembled product was dialyzed and transformed into NEB-10-Beta cells, incubated in a shaker for 1 hour, plated onto a KANA/CAM plate, and incubated overnight at 37° C. A single colony was used to inoculate a KANA/CAM culture and placed in a shaker incubator overnight at 37° C. Bacmid DNA was extracted from the culture by phenol/chloroform extraction. The extracted bacmid DNA was then screened using EcoNI and SbfI restriction enzymes for Rep78, and KpnI and BamHI for Rep52, to confirm successful cloning, and confirmed to lack mutations in the Rep78 and Rep52 genes by next-generation sequencing. As a control, the same Rep52 and Rep78/AAV1 capsid genes were cloned into an improved version of a bacmid comprising the wild-type AcMNPV-E2 genome which has improved multi-passage stability relative to the Control bacmid from Example 2, to generate Expression Construct 1. The Synbac2 transgene (SEAP) expressing vector was generated by Gibson Assembly™ of an ITR-SEAP-ITR fragment excised from a plasmid DNA backbone by PacI digest. The resultant bacmid DNA was screened by digesting with relevant restriction enzymes and confirmed to lack mutations in the SEAP transgene by next-generation sequencing.

Pre-baculovirus-infected insect cells (pre-BIICs) were prepared by mixing 200 ul Grace's insect media, 2 ul Mirus TransIT reagent, and 2 ul each of Synbac2-AAV1/Rep bacmid (or Expression Construct 1) and Synbac2-SEAP bacmid. The transfection complex was added to 30 ml of Sf9 cells at 1.5×10⁶ and incubated in a shaker overnight at 23° C. Cells were then frozen and stored as BIICs. The next day, BIICs were thawed and used in a TCID50 assay to determine viral titers. Viral titers were as follows: 1.3×10⁷ for Expression Construct 1, 4.2×10⁸ for Synbac2-AAV1/Rep, and 7.5×10⁸ for Synbac2-SEAP. Co-infection of BIICs Synbac2-AAV1/Rep and BIICs Synbac2-SEAP at MOI 0.002 for production of AAV was initiated with Sf9 cells at 3×10⁶ VCD. In parallel, Sf9 cells were co-infected with BIICs Expression Construct 1 (used in place of BIICs Synbac2-AAV1/Rep) and BIICs Synbac2-SEAP as a control for AAV production. Infected cells were lysed when viability was below 50%. 1 mL aliquots of each condition was collected starting from 2 days post-infection until day 5. A portion of the collected samples (100 ul) was lysed and used for Western blot analysis. The remainder of the sample was lysed by addition of 60 ul 10% Triton, 120 ul 2M arginine, and 10 U/ml benzonase. Clarified lysate from the lysed sample was used for quantitative polymerase chain reaction (qPCR) analysis.

Before initiating qPCR, rAAV samples were predigested with DNaseI to eliminate non capsid-associated DNA. DNaseI (2 U/ul, Teknova 3D1401) was diluted 30-fold in 1× DNAseI buffer (10 mM Tris-HCl, 2.5 mM MgCl2, 0.5 mM CaCl2), pH 7.6) and 95 ul volumes of the resulting DNAseI reaction buffer were combined with 5 ul volumes of rAAV samples. Samples were incubated for 1 h at 37° C. and then DNAseI digestions were stopped by adding 125 ul volumes of proteinase K/EDTA reaction buffer which contained 1 mg/ml proteinase K (Teknova P2050), 111 mM NaCl, 0.11% w/v sarkosyl, and 4 mM EDTA pH 8.0. After adding proteinase K/EDTA reaction buffer, samples were heated at 55° C. for 1 h followed by heat inactivation of proteinase K at 95° C. for 10 min. DNAseI/Proteinase K treated rAAV samples were then diluted 40-fold into 200 ul of 10 mM Tris pH 7.5. Four ul volumes of resulting tris diluted rAAV samples were added to 16 ul volumes of Q-PCR reaction mixes in 200-ul capacity, 96-well PCR plates. Ten-fold serially diluted DNA standards were included on the same PCR plates to enable quantification. All Q-PCR samples were run in triplicate. Q-PCR reaction mixes included TaqMan Fast Advanced Mastermix (ThermoFisher) diluted to 1.2× and 6 uM primers specific for the CMV promoter; Forward Primer (5′-TACGGTAAACTGCCCACTTG-3′; SEQ ID NO: 11), Reverse Primer (5′-GTCCCATAAGGTCATGTACTGG-3′; SEQ ID NO: 12) and Probe Primer (5′-FAM-GTCCCATAAGGTCATGTACTGG-ZEN-3′; SEQ ID NO: 13). Q-PCR reactions were done on a Lightcycler 480 (Roche), beginning with a 10 min, 95° C. heat denaturation, then 45 cycles of 95° C., 10 sec, 60° C., 10 sec, and a finishing step of 72° C., 10 sec. Resulting data were analyzed on MS-Excel.

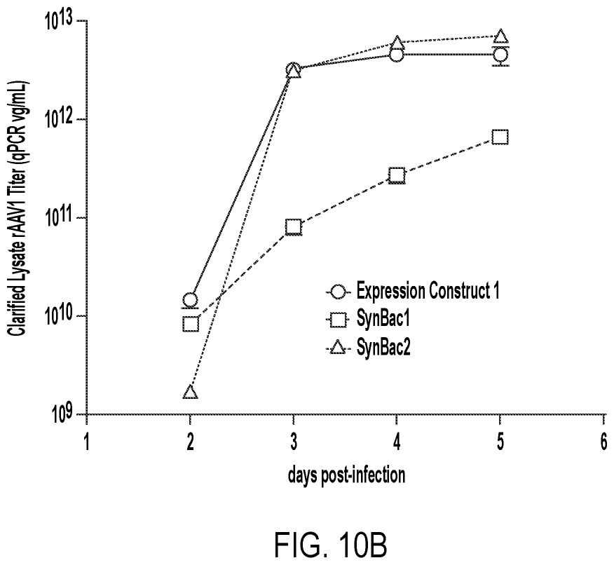

As shown in FIGS. 10A and 10B, comparable expression of rAAV1 proteins and rAAV1 production was achieved with Synbac2 relative to Expression Construct 1, as reflected by the amount of viral proteins produced and viral titer (vg/mL) determined by qPCR. As expected, Synbac1 showed a lower rAAV1 titer compared to both Synbac2 and Expression Construct 1. These results suggest that the synthetic baculovirus genome can support the production of AAV particles at comparable levels as a non-synthetic baculovirus genome, but with the significant advantages of being more modular and amenable to modification across multiple baculovirus loci.

1. A baculovirus expression construct comprising at least two subgenomic regions, wherein each subgenomic region comprises:
(i) a first unique junction and a second unique junction, wherein the first unique junction is present at the 5′ end of the subgenomic region, and the second unique junction is present at the 3′ end of the subgenomic region; and
(ii) a variant baculovirus nucleotide sequence comprising at least 5 fewer functional type IIS restriction enzyme sites, relative to the nucleotide sequence of a wild-type baculovirus genome;
wherein the baculovirus expression construct is replication-competent.

2. A plurality of fragments, wherein each fragment comprises:
(i) a unique 5′ overhang and a unique 3′ overhang; and
(ii) a variant baculovirus nucleotide sequence comprising at least 5 fewer functional restriction type IIS restriction enzyme sites, relative to the nucleotide sequence of a wild-type baculovirus genome.

3. A variant baculovirus genome which comprises a variant baculovirus nucleotide sequence comprising at least 5 fewer functional type IIS restriction enzyme sites, relative to the nucleotide sequence of a wild-type baculovirus genome, wherein the variant baculovirus genome is replication-competent.

4. (canceled)

5. The baculovirus expression construct of claim 1, wherein at least one, two, three, four or more of the at least 5 fewer functional type IIS restriction enzyme sites are different type IIS restriction enzyme sites, wherein:
(i) the first type IIS restriction enzyme site is recognized by a restriction enzyme selected from AcuI, AlwI, AaR1, BaeI, BbsI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, Earl, EciI, Esp3I, FauI, FokI, HgaI, HphI, HpyAV, MbolI, MlyI, MnlI, Nme-AIII, PaqCI, PleI, SapI, or SfaNI, or an isoschizomer of any of the preceding restriction enzymes; and
(ii) the second type IIS restriction enzyme site is recognized by a restriction enzyme selected from AcuI, AlwI, AaR1, BaeI, BbsI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI, BtsIMutI, CspCI, Earl, EciI, Esp3I, FauI, FokI, HgaI, HphI, HpyAV, MbolI, MlyI, MnlI, Nme-AIII, PaqCI, PleI, SapI, or SfaNI, or an isoschizomer of any of the preceding restriction enzymes.

6-7. (canceled)

8. The baculovirus expression construct of claim 1, which comprises:
(i) 2-20 subgenomic regions; or
(ii) at least subgenomic regions.

9. (canceled)

10. The baculovirus expression construct of claim 1, wherein the at least two subgenomic regions are formed by a first subgenomic fragment and a second subgenomic fragment, optionally wherein the at least two subgenomic regions further comprise one or more subfragments.

11. The baculovirus expression construct of claim 8, wherein the 2-20 subgenomic regions are formed by 2-20 subgenomic fragments, optionally wherein the 2-20 subgenomic fragments further comprise at least 1-20 subfragments.

12-13. (canceled)

14. The baculovirus expression construct of claim 11, wherein:
(i) each of the first subgenomic fragment and the second subgenomic fragment comprise a unique 5′ overhang and a unique 3′ overhang;
(ii) each of the 2-20 subgenomic fragments comprise a unique 5′ overhang and a unique 3′ overhang; and/or
(ii) each of the at least 1-20 subfragments comprises a unique 5′ overhang and a unique 3′ overhang.

15. The baculovirus expression construct of claim 14, wherein:
(i) the first unique junction comprises the unique 5′ overhang of the first subgenomic fragment and the unique 3′ overhang of the second subgenomic fragment, wherein the unique 5′ overhang of the first subgenomic fragment is partially complementary or fully complementary to the unique 3′ overhang of the second subgenomic fragment;
(ii) the unique 5′ overhang of a first subfragment is partially complementary or fully complementary to the unique 3′ overhang of the second subfragment; and/or
(iii) the unique 5′ overhang of one fragment of the plurality is complementary or fully complementary to the unique 3′ overhang of another fragment of the plurality.

16. (canceled)

17. The baculovirus expression construct of claim 14, wherein the unique 5′ overhang and the unique 3′ overhang:
(i) each independently comprise at least 1-6 nucleotides in length (e.g., 4 nucleotides);
(ii) comprise cohesive ends; and/or
(iii) each comprise a nucleotide sequence independently selected from ACAA, GGTC, GACC, CCAG, CTGG, CCTT, AAGG, TCAT, ATGA, TCGC, GCGA, AGAG, CTCT, AACT, AGTT, CGGT, ACCG, ATAC, GTAT, GAGT, ACTC, TTCC, GGAA, ATTA, TAAT, TCCT, AGGA, TCTA, TAGA, TGTA, TACA, GATG, CATC, or TTGT, wherein the sequences of the unique 5′ overhang and 3′ overhang in a subgenomic fragment are different and/or wherein the sequences of the unique 5′ overhang and 3′ overhang in a fragment are different.

18. The baculovirus expression construct of claim 14, wherein:
(i) the same type IIS restriction enzyme is used to generate the unique 5′ overhang and the unique 3′ overhang of each subgenomic fragment; and/or
(ii) the same type IIS restriction enzyme is used to generate the unique 5′ overhang and the unique 3′ overhang of each subfragment, wherein the type IIS restriction enzyme used to generate the unique 5′ overhang and the unique 3′ overhang of each subgenomic fragment differs from the type IIS restriction enzyme used to generate the subgenomic fragments.

19-25. (canceled)

26. The baculovirus expression construct of claim 14, wherein:
(i) the subgenomic fragments are capable of ordered assembly based on the complementarity of the 5′ overhang in one subgenomic fragment with the 3′ overhang in another subgenomic fragment to generate a variant baculovirus genome or the baculovirus expression construct; and/or
(ii) the at least 1-20 subfragments are capable of ordered assembly based on the complementarity of the 5′ overhang in one subfragment with the 3′ overhang in another subfragment to generate the subgenomic fragments and/or the baculovirus expression construct.

27. The baculovirus expression construct of claim 12, wherein:
(i) at least two of the subgenomic fragments are covalently linked, such that at least two subgenomic regions are formed and/or to generate a variant baculovirus genome or the baculovirus expression construct, optionally wherein the subgenomic fragments are ligated in a single step to generate a variant baculovirus genome or the baculovirus expression construct;
(ii) the 2-20 subgenomic fragments are covalently linked such that 2-20 subgenomic regions are formed and/or to generate a variant baculovirus genome or the baculovirus expression construct, optionally wherein the subgenomic fragments are ligated in a single step to generate a variant baculovirus genome or the baculovirus expression construct; and/or
(iii) the at least 1-20 subfragments are covalently linked such that one or more subgenomic regions are formed and/or to generate the baculovirus expression construct, optionally wherein the subfragments are ligated in a single step to generate one or more subgenomic fragments (or subgenomic regions), a variant baculovirus genome or the baculovirus expression construct.

28-30. (canceled)

31. The baculovirus expression construct of claim 11, wherein one, two, or all of:
(i) one or more of the 2-20 subgenomic fragments comprise a heterologous nucleotide sequence from the variant baculovirus nucleotide sequence; or
(ii) one or more of the at least 1-20 subfragments comprise a heterologous nucleotide sequence from the variant baculovirus nucleotide sequence
wherein the heterologous nucleotide sequence comprises a nucleotide sequence encoding a polypeptide of interest or a nucleotide sequence of interest.

32-35. (canceled)

36. The baculovirus expression construct of claim 11, wherein one or more of the subgenomic fragments and/or one or more of the subfragments encodes an AAV1 capsid protein or a variant thereof, an AAV2 capsid protein or a variant thereof, an AAV3 capsid protein or a variant thereof, an AAV4 capsid protein or a variant thereof, an AAV5 capsid protein or a variant thereof, an AAV6 capsid protein or a variant thereof, an AAV8 capsid protein or a variant thereof, an AAV9 capsid protein or a variant thereof, or an AAVrh10 capsid protein or a variant thereof.

37. (canceled)

38. The baculovirus expression construct of claim 12, wherein:
(i) the variant baculovirus nucleotide sequence of one or more of the subgenomic fragments comprises a modification; and/or
(ii) the variant baculovirus nucleotide sequence of one or more of the at least 1-20 subfragments comprises a modification,
wherein the modification is present in a non-essential gene or a regulatory region of a non-essential gene, optionally wherein the modification results in inactivation of the non-essential gene or the regulatory region of a non-essential gene.

39. (canceled)

40. The baculovirus expression construct of claim 38, wherein the non-essential gene is selected from one, two, three, or more of egt, p74 (PIF0), p26, SOD, ChiA, v-cath, p10, polyhedrin, ctx, odv-e56, PIF1, PIF2, PIF3, PIF4, PIF5, Tn7, AcORF-91, AcORF-108, AcORF-52, v-ubi, or p94.

41-43. (canceled)

44. The baculovirus expression construct of claim 1, which comprises a baculovirus genome or a portion thereof, wherein the baculovirus genome or portion thereof is:
(i) is at least 10 kb-140 kb in length;
(ii) comprises a nucleotide sequence or a portion thereof from a baculovirus genome selected from Autographa californica multiple nucleopolyhedrovirus (AcMNPV), Bombyx mori nucleopolyhedrovirus (BmNPV), Anticarsia gemmatalis nucleopolyhedrovirus (AgMNPV), Orgvia pseudotsugata nucleopolyhedrovirus (OpMNPV), Thysanoplusia orichalcea nucleopolyhedrovirus (ThorMNPV), or a variant thereof;
(iii) comprises a wild-type baculovirus genome or a modified baculovirus genome: or
(iv) is a bMON14272 baculovirus genome.

45-48. (canceled)

49. A vector comprising the baculovirus expression construct of claim 1.

50. (canceled)

51. A bacterial artificial chromosome (BAC) which comprises at least 5 fewer functional type IIS restriction enzyme sites, relative to a reference BAC.

52. A cell comprising the baculovirus expression construct of claim 1, optionally wherein the cell is an insect cell , a mammalian cell, or a bacterial cell.

53. A composition comprising the baculovirus expression construct of claim 1, and a carrier.

54. A kit comprising the baculovirus expression construct of claim 1, and instructions for use, optionally wherein the kit further comprises a type IIS restriction enzyme.

55. A method of generating a variant baculovirus genome, comprising:
(i) providing the plurality of fragments of claim 2;
(ii) introducing a modification into one or more fragments comprising the variant baculovirus nucleotide sequence; and
(iii) incubating the plurality of fragments under conditions suitable to form a variant baculovirus genome;
thereby generating the variant baculovirus genome.

56. (canceled)
